(19) United States
(12) Patent Application Publication
(10) Pub. No.: US 2001/0051335 A1
(43) Pub. Date: Dec. 13, 2001

(54) POLYNUCLEOTIDES AND POLYPEPTIDES DERIVED FROM CORN TASSEL

(76) Inventors: RAGHUNATH V. LALGUDI, CLAYTON, MO (US); LAURA Y. ITO, PLEASANTON, CA (US); BRADLEY K. SHERMAN, OAKLAND, CA (US)

Correspondence Address: Christopher Turner, LEGAL DEPARTMENT, INCYTE GENOMICS, INC., 3160 PORTER DRIVE, PALO ALTO, CA, 94304, US

(21) Appl. No.: 09/294,093

(22) Filed: Apr. 16, 1999

Related U.S. Application Data
(60) Provisional application No. 60/082,567, filed on Apr. 21, 1998.

(51) Int. Cl. C12Q 1/68; C12P 21/06

(52) U.S. Cl. 435/6; 435/69.1

(57) ABSTRACT

The present invention provides purified, corn tassel-derived polynucleotides (cdps) which encode corn tassel-derived polypeptides (CDPs). The invention also provides for the use of cdps or their complements, oligonucleotides, or fragments in methods for determining altered gene expression, to recover regulatory elements, and to follow inheritance of desirable characteristics through hybrid breeding programs. The invention further provides for vectors and host cells containing cdps for the expression of CDPs. The invention additionally provides for (i) use of isolated and purified CDPs to induce antibodies and to screen libraries of compounds and (ii) use of anti-CDP antibodies in diagnostic assays.

This application claims the benefit of U.S. Provisional Application No. 60/082,567, our Docket No. PL-0009 P, filed on Apr. 21, 1998.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention relates to nucleic and amino acid sequences derived from corn tassel and to the use of these sequences in the identification, evaluation, and alteration of desired characteristics associated with growth and development, disease resistance, environmental adaptability, quality, and yield.

### BACKGROUND OF THE INVENTION

The field of plant breeding deals with the manipulation of plant genomes with the purpose of improving characteristics of the plant. Plant breeders use data and methodology from plant physiology, genetics, biochemistry, pathology, statistics, and molecular biology. One of the most improved hybrid crops is corn, Zea mays (L.). Corn is presently the second-most economically important crop in the United States. Acreage of field corn (used for livestock feed, corn starch, corn syrup, fuel ethanol, and oil) and sweet corn (used fresh or processed for human consumption) exceeds that of any other agronomic crop. Annual losses, reduction in quality and yield, due to diseases and infestation may range from 7 to 17%. Studies of corn may be used as a model for other economically important agronomic grasses.

Corn is a monocotyledonous plant which has one seed leaf, uses the C₄ photosynthetic pathway, and has scattered vascular bundles. The mature plant is made up of roots, stem, leaves, and reproductive structures. The root system functions to anchor the plant and to absorb water and nutrients. The corn stem consists of a series of nodes, each bearing one bladelike leaf with parallel veins, and internodes, elongated stem sections. The stem and its leaves are commonly referred to as a stalk. Leaves arise alternately and are arranged in two rows along either side of the stem. The male reproductive structure is the tassel, and its flowers produce pollen. The female reproductive structure is the ear, and its flowers each have a silk for pollination. When a pollen grain is shed onto a silk and germinates, a pollen tube grows down the silk and delivers two sperm to the female gametophyte. Within the gametophyte, one sperm fertilizes the egg, and the other, two polar nuclei. The embryo and endosperm produced by this double fertilization develop into the seed which matures in about two months.

The vegetative (V) and reproductive (R) stages of growth for a corn plant are as follows: VE-emergence from the soil of the seedling leaf; V1-first true leaf; V2-second leaf; . . . V(n)-nth leaf; VT-tasseling stage; and reproductive stages, R1-silking; R2-blister; R3-milk; R4-dough; R5-dent; and R6-physiological maturity (Ritchie, S. W. et al. (1986) How a Corn Plant Develops, Iowa State University Cooperative Extension Service, Ames IA 48:1-21).

Corn Tassel

The tassel is initiated early in corn plant development. By the V5 stage, the microscopic tassel is present at the tip of the stem apex; by V6, it is above ground; by V9, it begins rapid development; and by V17, it is visible emerging from the top of the plant. At VT, the last branch of the tassel is completely visible, and silks have not yet emerged from the ear. When the male flowers mature at R0; anthers emerge from the spikelet, open, and release pollen which is dispersed by wind or insects. Depending on environmental conditions, an individual tassel may take as long as seven days to finish shedding pollen, and a whole field may take as long as 14 days to complete pollen shed.

Male flowers contain stamens; each composed of a filament which bears a bilobate anther. The filament contains a single vascular bundle for conducting water and nutrients to the lobes of the anther. Each lobe has two locules or cavities where the microsporangia or pollen are formed. The cell layers of the anther are epidermis, endothecium, and tapetum, a nutritive tissue of multinucleate cells which contribute to pollen development.

Microspore mother cells are closely packed within the locules and each microspore mother cell undergoes meiosis to produce four haploid microspores which are subsequently walled off as individual pollen grains. Within each pollen grain, the haploid nucleus undergoes mitosis to produce the pollen tube nucleus which guides pollen tube germination and growth through the silk to the egg, and two sperm nuclei which carry out the double fertilization of the egg and endosperm.

Problems Of Corn Tassels

All parts of the corn plant are susceptible to diseases, insect infestations, and stress. These conditions are usually diagnosed by their above-ground leaf, stem, fruit and/or seed symptoms and are caused by fungi, bacteria, mycoplasmas and related organisms, viruses, nematodes, parasitic seed plants, and abnormal environmental conditions.

Stewart's bacterial wilt caused by Erwinia stewartii is common in eastern North America and has been reported in eastern Europe and Italy. Field corn is genetically more resistant to bacterial wilt than sweet corn. Infected sweet corn hybrids wilt rapidly and their leaves show pale green to yellow streaks with irregular margins which soon become dry and brown. The bacteria which may spread to all parts of the plant may produce premature, bleached, and dead tassels which gravely affect yield. E. stewartii is identified in the field by plant symptoms and the yellow ooze which comes from cut ends of infected stems or leaves. E. stewartii is spread by beetles; however, only the corn flea beetle (Chaetocnema pulicaria) overwinters and spreads the bacteria the next growing season. Mineral nutrition also influences susceptibility to infection; nitrogen in the form of ammonium and phosphorus increase susceptibility, and high calcium and potassium levels decrease susceptibility. Disease severity may also be aggravated by high temperatures.

Fungal diseases are spread by spores that germinate under favorable conditions of temperature and moisture. Spores germinate to produce branched threads called hyphae that infect plants by direct penetration of the epidermis or through natural openings or wounds. Resting bodies (chlamydospores, sclerotia) allow fungi to survive under unfavorable conditions for long periods in the soil or in plant debris.

Crazy Top is caused by the downy mildew, Sclerophthora macrospora (Phytophthora macrospora), and is widespread in the US. Although symptoms vary greatly, tassels (and ears) generally develop a mass of leafy structures (“crazy top”), and no pollen is formed In addition, severely infected plants may be stunted, have excessive suckering and display narrow straplike leaves with yellow stripes. Motile spores of the fungus germinate in water-saturated soils, penetrate corn tissues, and form abundant mycelium in the meristems. New fungal fruiting bodies are produced by infected tissues and overwinter in soil and infected debris. Crazy Top may cause substantial losses in localized areas. Other important fungal diseases of corn include tassel rots, stock rots, anthracnose, smuts, rusts, Helminthosporium (Bipolaris) leaf spots and blights, and other downy mildews.

Viruses are submicroscopic particles composed of nucleic acid and protein which are transmitted by biological vectors, e.g., aphids, leafhoppers, planthoppers, beetles, and other insects. A few corn viruses are seed-transmitted or overwinter in weed and crop plants or insect bodies. Viruses induce a variety of local or systematic symptoms (e.g., mosaic patterns, yellowing or reddening of the foliage, stunting, ringspots, and necrosis) which may be exacerbated by environmental conditions. Viruses which affect field and sweet corn include maize chlorotic dwarf; maize chlorotic mottle; maize dwarf mosaic; and corn lethal necrosis (a complex of maize chlorotic mottle virus and maize dwarf mosaic).

Maize Dwarf Mosaic Virus (MDMV) is a long, flexuous, rod-shaped virus. It is transmitted on seed or by over 20 species of aphids which acquire the virus from other grasses and transmit it to corn. Plants may be slightly stunted, and reduction in ear size and seed set may occur. MDMV symptoms are highly variable and include narrow, light green or yellowish streaks along the leaf veins and/or dark green “islands” on a yellow background. Symptoms may be present on all leaves and husks that develop following infection. Both environmental conditions and the developmental stage at which the plant is infected may cause a reduction in yield; infection of young plants early in the season causes the most significant losses. In addition, early infection may predispose corn to root and stalk rots and premature death.

Corn lethal necrosis, found in Kansas and Nebraska, results from the synergistic interaction of maize chlorotic mottle virus (MCMV) in combination with either MDMV strain-A or -B or wheat streak mosaic virus (WSMV). Yield loss greatly exceeds that predicted from the cumulative effects of the individual viruses and may approach 100%. Symptoms include severe mottling, browning of the tassel and leaves, and early death of the plant. Late infections cause ears to fill incompletely and husks to dry prematurely. Potential vectors of these viruses may be: corn rootworm beetles (Diabrotica spp.) for MCMV, greenbug (Schizaphi graminum) and aphids (Rhopalosiphum maidis) for MDMV, and wheat curl mite (Eriophyes tulipae) for WSMV.

Mycoplasmas and spiroplasmas are minute, pleomorphic organisms that lack a rigid cell wall and are related to gram-positive bacteria. Evidence from electron microscopy and administration of antibiotics indicates that organisms closely resembling mycoplasmas are involved in many plant diseases. These mycoplasma-like organisms (MLO) have not been completely characterized; however, most diseases now thought to be caused by MLO or spiroplasmas are yellows-type diseases. Like some viruses, they occur only in the phloem of diseased plants and are transmitted by leafhoppers, dodder (Cuscuta spp), grafting, and propagation of infected plant parts. MLO and spiroplasmas produce viruslike symptoms including chlorosis or yellowing, stunting, phyllody, proliferation of axillary shoots, and sterility (e.g., corn stunt). Unlike viruses, these organisms are sensitive to the tetracyclines. Characteristic symptoms include yellowing, shortening of the internodes, phyllody, a bushy morphology, sterility, virescence, and reduction of root growth. An example of an MLO-associated disease is Maize Bushy Stunt. This disease was first described in the southern USA where it is spread by leafhoppers. Symptoms include yellowing or reddening, tearing, twisting, and shortening of leaves. The numerous shoots which develop at the leaf nodes and base of the plant give the plant a stunted, bushy morphology.

Important pests of corn include European corn, southwestern corn, and common stalk borers; corn tasselworm; chinch bug; corn ear worm; armyworm; grasshoppers; corn leaf aphid; corn flea, dusky sap, and Japanese beetles; and spider mites. The Dusky Sap Beetle (Carpophilus luqubris) is dark gray or brown, oblong to oval in shape, has club-like antennae and is between 3 and 4 mm long. It is particularly common along the Atlantic coast of the US. Sap beetles are attracted to sweet corn as it tassels, and they feed on both the tassels and on ripening pollen. Later the beetles move to leaf axils where pollen has fallen and collected and lay eggs in the developing ear kernels. At least three or four generations of the beetles occur each year, and more generations are possible if beetles overwinter in decaying vegetation or debris buried in the soil.

Environmental conditions also affect growth, development, and yield by altering pathogen activity or host physiology. The severity of the excess, deficiency, or imbalance of soil nutrients or water; extreme soil acidity or alkalinity; very high or low temperatures; air pollutants; or mechanical, pesticide, or other injury varies with the stage of plant maturity during which the disturbance occurs and the plant organ involved. Extended periods of hot, dry winds may cause tassel blasting and loss of pollen.

Corn Disease Control

To control corn diseases, it is necessary to disrupt disease development. Intervention requires understanding the pathogens, the spread of pathogens, disease cycles, and plant resistance. Disease control may be achieved by a single procedure (chemical sprays) or by the integrated use of environmental, genetic, and chemical factors. Successful, long-term disease control generally includes planting disease-resistant varieties, applying chemical control, and implementing sanitary cultural methods.

### SUMMARY OF THE INVENTION

The present invention provides nucleic acid sequences comprising corn tassel-derived polynucleotides (cdps) as presented in the Sequence Listing. Some of the cdps uniquely identify structural, functional, and regulatory genes of the corn tassel. The invention encompasses oligonucleotides, fragments, and derivatives of the cdps and provides nucleic acid sequences complementary to the nucleic acid sequences listed in the Sequence Listing.

The present invention further provides the following cdps of particular interest as identified by SEQ ID NO: 1615 (700344704H1), SEQ ID NO: 2691 (700346502H1), SEQ ID NO: 2813 (700346702H1), SEQ ID NO: 3616 (700380601H1), SEQ ID NO: 3681 (700380774H1), SEQ ID NO: 233 (700342388H1), SEQ ID NO: 2060 (700345470H1), SEQ ID NO: 964 (700343568H1), SEQ ID NO: 2811 (700346695H1), SEQ ID NO: 2519 (700346233H1), SEQ ID NO: 2083 (700345510H1), SEQ ID NO: 1101 (700343780H1), SEQ ID NO: 1847 (700345052H1), SEQ ID NO: 1777 (700344946H1), SEQ ID NO: 1780 (700344949H1), SEQ ID NO: 1733 (700344870H1), SEQ ID NO: 3018 (700347036H1), SEQ ID NO: 3158 (700347281H1), SEQ ID NO: 2220 (700345749H1), SEQ ID NO: 165 (700342268H1), SEQ ID NO: 139 (700342223H1), SEQ ID NO: 2823 (700346721H1), SEQ ID NO: 2065 (700345477H1), SEQ ID NO: 605 (700343040H1), SEQ ID NO: 3618 (700380603H1), SEQ ID NO: 1537 (700344525H1), SEQ ID NO: 469 (700342790H1), SEQ ID NO: 3344 (700379867H1), SEQ ID NO: 180 (700342291H1), SEQ ID NO: 303 (700342521H1), SEQ ID NO: 1249 (700344013H1), SEQ ID NO: 1036 (700343679H1), SEQ ID NO: 2920 (700346877H1), SEQ ID NO: 3619 (700380604H1), SEQ ID NO: 1061 (700343721H1), SEQ ID NO: 1589 (700344654H1), SEQ ID NO: 3100 (700347193H1), SEQ ID NO: 2182 (700345675H1), SEQ ID NO: 2218 (700345743H1), SEQ ID NO: 398 (700342679H1), SEQ ID NO: 175 (700342283H1), SEQ ID NO: 3533 (700380411H1), SEQ ID NO: 3615 (700380596H1), SEQ ID NO: 89 (700281941H2), SEQ ID NO: 3530 (700380405H1), SEQ ID NO: 3023 (700347043H1), SEQ ID NO: 3185 (700347339H1), SEQ ID NO: 3062 (700347112H1), SEQ ID NO: 1082 (700343752H1), SEQ ID NO: 55 (700281888H2), SEQ ID NO: 2955 (700346930H1), SEQ ID NO: 1032 (700343674H1), SEQ ID NO: 163 (700342263H1), SEQ ID NO: 2864 (700346807H1), SEQ ID NO: 1286 (700344069H1), SEQ ID NO: 2560 (700346291H1), SEQ ID NO: 742 (700343247H1), SEQ ID NO: 1731 (700344866H1), SEQ ID NO: 370 (700342630H1), SEQ ID NO: 1167 (700343872H1), SEQ ID NO: 715 (700343202H1), SEQ ID NO: 3314 (700379793H1), SEQ ID NO: 2156 (700345638H1), SEQ ID NO: 624 (700343071H1), SEQ ID NO: 545 (700342924H1), SEQ ID NO: 2855 (700346794H1), SEQ ID NO: 850 (700343408H1), SEQ ID NO: 1557 (700344614H1), SEQ ID NO: 1872 (700345114H1), SEQ ID NO: 1000 (700343626H1), SEQ ID NO: 1045 (700343695H1), SEQ ID NO: 2753 (700346604H1), SEQ ID NO: 70 (70028191(H1), SEQ ID NO: 1235 (700343988H1), SEQ ID NO: 389 (700342664H1), SEQ ID NO: 486 (700342817H1), SEQ ID NO: 766 (700343281H1), SEQ ID NO: 1310 (700344105H1), SEQ ID NO: 3202 (700347368H1), SEQ ID NO: 1941 (700345253H1), SEQ ID NO: 6 (700281814H2), SEQ ID NO: 1884 (700345138H1), SEQ ID NO: 2316 (700345916H1), SEQ ID NO: 1948 (700345263H1), SEQ ID NO: 2277 (700345849H1), SEQ ID llO: 2019 (700345394H1), SEQ ID NO: 1696 (700344820H1), SEQ ID NO: 226 (700342373H11), SEQ ID NO: 262 (700342448H1), SEQ ID NO: 1377 (700344208H1), SEQ ID NO: 1279 (700344059H1), SEQ ID NO: 1770 (700344935H1), SEQ ID NO: 532 (700342903H1), SEQ ID NO: 843 (700343396H1), SEQ ID NO: 360 (700342616H1), SEQ ID NO: 540 (700342915H1), SEQ ID NO: 413 (700342708H1), SEQ ID NO: 1252 (700344017H1), SEQ ID NO: 1560 (700344618H1), SEQ ID NO: 3541 (700380432H1), SEQ ID NO: 243 (700342413H1), SEQ ID NO: 1938 (700345249H1), SEQ ID NO: 1127 (700343816H1), SEQ ID NO: 1545 (700344541H1), SEQ ID NO: 1946 (700345258H1), SEQ ID NO: 298 (700342510H1), SEQ ID NO: 2906 (700346860H1), SEQ ID NO: 783 (700343310H1), SEQ ID NO: 616 (700343060H1), SEQ ID NO: 2097 (700345529H1), SEQ ID NO: 2393 (700346036H1), SEQ ID NO: 860 (700343420H1), SEQ ID NO: 2196 (700345705H1), SEQ ID NO: 2403 (700346049H1), SEQ ID NO: 2309 (700345906H1), SEQ ID NO: 1963 (700345289H1), SEQ ID NO: 1383 (700344218H1), SEQ ID NO: 3201 (700347367H1), SEQ ID NO: 3589 (700380530H1), SEQ ID NO: 660 (700343124H1), SEQ ID NO: 2254 (700345807H1), SEQ ID NO: 2689 (700346496H1), SEQ ID NO: 3166 (700347296H1), SEQ ID NO: 2023 (700345404H1), SEQ ID NO: 726 (700343218H1), SEQ ID NC: 363 (700342620H1), SEQ ID NO: 2968 (700346951H1), SEQ ID NO: 2214 (700345737H1), SEQ ID NO: 989 (700343607H1), SEQ ID NO: 2642 (700346426H1), SEQ ID NO: 2741 (700346581H1), SEQ ID NO: 1321 (700344122H1), SEQ ID NO: 1255 (700344022H1), SEQ ID NO: 2311 (700345908H1), SEQ ID NO: 1857 (700345075H1), SEQ ID NO: 2571 (700346316H1), SEQ ID NO: 3063 (700347116H1), SEQ ID NO: 1649 (700344750H1), SEQ ID NO: 1653 (700344754H1), SEQ ID NO: 1762 (700344924H1), SEQ ID NO: 1985 (700345329H1), SEQ ID NO: 2382 (700346023H1), SEQ ID NO: 2468 (700346152H1), SEQ ID NO: 1567 (700344628H1), SEQ ID NO: 991 (700343609H1), SEQ ID NO: 2124 (700345577H1), SEQ ID NO: 829 (700343377H1), SEQ ID NO: 3623 (700380616H1), SEQ ID NO: 725 (700343216H1), SEQ ID NO: 1676 (700344785H1), SEQ ID NO: 399 (700342680H1), SEQ ID NO: 2790 (700346658H1), SEQ ID NO: 2284 (700345859H1), SEQ ID NO: 2838 (700346745H1), SEQ ID NO: 757 (700343266H1), SEQ ID NO: 1251 (700344016H1), SEQ ID NO: 207 (700342338H1), SEQ ID NO: 3515 (700380270H1), SEQ ID NO: 3224 (700379610H1), SEQ ID NO: 2958 (700346935H1), SEQ ID NO: 1861 (700345079H1), SEQ ID NO: 3052 (700347092H1), SEQ ID NO: 2542 (700346264H1), SEQ ID NO: 3602 (700380571H1), SEQ ID NO: 2788 (700346656H1), SEQ ID NO: 1966 (700345293H1), SEQ ID NO: 1813 (700345004H1), SEQ ID NO: 573 (700342936H11), SEQ ID NO: 3572 (700380488H1), SEQ ID NO: 1368 (700344191H1), SEQ ID NO: 774 (700343291H1), SEQ ID NO: 443 (700342747H1), SEQ ID NO: 1083 (700343755H1), SEQ ID NO: 110 (700281973H1), SEQ ID NO: 118 (700281986H2), SEQ ID NO: 2088 (700345516H1), SEQ ID NO: 2932 (700346894H1), SEQ ID NO: 2815 (700346704H1), SEQ ID NO: 1112 (700343793H1), SEQ ID NO: 3131 (700347236H1), SEQ ID NO: 682 (700343154H1), SEQ ID NO: 1965 (700345291H1), SEQ ID NO: 1866 (700345094H1), SEQ ID NO: 898 (700343475H1), SEQ ID NO: 3582 (700380514H1), SEQ ID NO: 916 (700343509H1), SEQ ID NO: 188 (700342309H1), SEQ ID NO: 1364 (700344L86H1), SEQ ID NO: 1421 (700344295H1), SEQ ID NO: 3685 (700380786H1), SEQ ID NO: 2404 (700346050H1), SEQ ID NO: 3246 (700379650H1), SEQ ID NO: 1673 (700344782H1), SEQ ID NO: 953 (700343552H1), SEQ ID NO: 2216 (700345739H1), SEQ ID NO: 1232 (700343985H1), SEQ ID NO: 775 (700343292H1), SEQ ID NO: 869 (700343431H1), SEQ ID NO: 145 (700342231H1), SEQ ID NO: 2927 (700346887H1), SEQ ID NO: 1820 (700345013H1), SEQ ID NO: 2244 (700345785H1), SEQ ID NO: 1078 (700343746H1), SEQ ID NO: 2965 (700346943H1), SEQ ID NO: 1085 (700343757H1), SEQ ID NO: 730 (700343225H1), SEQ ID NO: 792 (700343325H1), SEQ ID NO: 3218 (700347395H1), SEQ ID NO: 3033 (700347064H1), SEQ ID NO: 1058 (700343715H1), SEQ ID NO: 302 (700342515H1), SEQ ID NO: 2384 (700346026H1), SEQ ID NO: 1220 (700343965H1), SEQ ID NO: 1883 (700345136H1), SEQ ID NO: 1928 (700345235H1), SEQ ID NO: 2598 (700346351H1), SEQ ID NO: 315 (700342543H1), SEQ ID NO: 1405 (700344265H1), SEQ ID NO: 3128 (700347232H1), SEQ ID NO: 942 (700343540H1), SEQ ID NO: 1109 (700343790H1), SEQ ID NO: 1490 (700344425H1), SEQ ID NO: 3040 (700347072H1), SEQ ID NO: 2411 (700346060H1), SEQ ID NO: 2477 (700346167H1), SEQ ID NO: 446 (700342755H1), SEQ ID NO: 3026 (700347050H1), SEQ ID NO: 3554 (700380452H1), SEQ ID NO: 3465 (700380146H1), SEQ ID NO: 143 (700342228H1), SEQ ID NO: 867 (700343428H1), SEQ ID NO: 1139 (700343832H1), SEQ ID NO: 3067 (700347126H1), SEQ ID NO: 799 (700343333H1), SEQ ID NO: 2059 (700345469H1), SEQ ID NO: 2120 (700345571H1), SEQ ID NO: 2179 (700345671H1), SEQ ID NO: 2707 (700346525H1), SEQ ID NO: 967 (700343572H1), SEQ ID NO: 1356 (700344174H1), SEQ ID NO: 2896 (700346848H1), SEQ ID NO: 304 (700342523H1), SEQ ID NO: 1063 (700343723H1), SEQ ID NO: 560 (700342959H1), SEQ ID NO: 2625 (700346389H1), SEQ ID NO: 2501 (700346210H1), SEQ ID NO: 319 (700342551H1), SEQ ID NO: 3173 (700347308H1), SEQ ID NO: 2313 (700345910H1), SEQ ID NO: 2022 (700345403H1), SEQ ID NO: 1076 (700343742H1), SEQ ID NO: 3025 (700347049H1), SEQ ID NO: 1719 (700344849H1), SEQ ID NO: 3342 (700379862H1), SEQ ID NO: 643 (700343103H1), SEQ ID NO: 3649 (700380682H1), SEQ ID NO: 3613 (700380589H1), SEQ ID NO: 807 (700343342H1), SEQ ID NO: 1760 (700344920H1), SEQ ID NO: 2558 (700346288H1), SEQ ID NO: 492 (700342832H1), SEQ ID NO: 504 (700342846H1), SEQ ID NO: 172 (700342278H1), SEQ ID NO: 1738 (700344878H1), SEQ ID NO: 2014 (700345384H1), SEQ ID NO: 390 (700342666H1), SEQ ID NO: 401 (700342682H1), SEQ ID NO: 3512 (700380263H1), SEQ ID NO: 3441 (700380096H1), SEQ ID NO: 2370 (700346008H1), SEQ ID ND: 1323 (700344124H1), SEQ ID NO: 634 (700343087H1), SEQ ID NO: 1986 (700345330H1), SEQ ID NO: 238 (700342403H1), SEQ ID NO: 987 (700343603H1), SEQ ID NO: 273 (700342463H1), SEQ ID NO: 208 (700342340H1), SEQ ID NO: 2674 (700346475H1), SEQ ID NO: 3119 (700347222H1), SEQ ID NO: 2203 (700345716H1), SEQ ID NO: 2186 (700345686H1), SEQ ID NO: 1307 (700344096H1), SEQ ID NO: 1372 (700344196H1), SEQ ID NO: 3053 (700347093H1), SEQ ID NO: 2507 (700346218H1), SEQ ID NO: 1049 (700343703H1), SEQ ID NO: 391 (700342667H1), SEQ ID NO: 3092 (700347176H1), SEQ ID NO: 3389 (700379975H1), SEQ ID NO: 326 (700342560H1), SEQ ID NO: 3605 (700380575H1), SEQ ID NO: 2946 (700346918H1), SEQ ID NO: 907 (700343487H1), SEQ ID NO: 3366 (700379925H1), SEQ ID NO: 1237 (700343991H1), SEQ ID NO: 3610 (700380584H1), SEQ ID NO: 2464 (700346146H1), SEQ ID NO: 3650 (700380683H1), SEQ ID NO: 2024 (700345405H1) SEQ ID NO: 1145 (700343841H1), SEQ ID NO: 1590 (700344655H1), SEQ ID NO: 2219 (700345747H1), SEQ ID NO: 224 (700342363H1), SEQ ID NO: 1108 (700343789H1), SEQ ID NO: 344 (700342589H1), SEQ ID NO: 2995 (700346996H1), SEQ ID NO: 3282 (700379719H1), SEQ ID NO: 1826 (700345020H1), SEQ ID NO: 1443 (700344334H1), SEQ ID NO: 3021 (700347040H1), SEQ ID NO: 558 (700342952H1), SEQ ID NO: 2292 (700345873H1), SEQ ID NO: 2030 (700345412H1), SEQ ID NO: 1005 (700343632H1), SEQ ID NO: 254 (700342432H1), SEQ ID NC): 1506 (700344454H1), SEQ ID NO: 1146 (700343842H1), SEQ ID NO: 1756 (700344911H1), SEQ ID NO: 2471 (700346157H1), SEQ ID NO: 1170 (700343875H1), SEQ ID NO: 1765 (700344928H1), SEQ ID NO: 1585 (700344649H1), SEQ ID NO: 495 (700342835H1), SEQ ID NO: 392 (700342670H1), SEQ ID NO: 5179 (700355947H1), SEQ ID NO: 4225 (700353526H1), SEQ ID NO: 5639 (700382063H1), SEQ ID NO: 3797 (700352605H1), SEQ ID NO: 4188 (700353271H1), SEQ ID NO: 5940 (700382691H1), SEQ ID NO: 4388 (700353955H1), SEQ ID NO: 5962 (700382739H1), SEQ ID NO: 6034 (700382890H1), SEQ ID NO: 4988 (700355520H1), SEQ ID NO: 5105 (700355788H1), SEQ ID NO: 5943 (700382702H1), SEQ ID NO: 4276 (700353625H1), SEQ ID NO: 5267 (700356140H1), SEQ ID NO: 3698 (700282414H2), SEQ ID NO: 4942 (700355431H1), SEQ ID NO: 4803 (700355029H1), SEQ ID NO: 5171 (700355925H1), SEQ ID NO: 5366 (700356466H1), SEQ ID NO: 4940 (700355428H1), SEQ ID NO: 5895 (700382605H1), SEQ ID NO: 4403 (700354129H1), SEQ ID NO: 4830 (700355088H1), SEQ ID NO: 5695 (700382183H1), SEQ ID NO: 5440 (700356708H1), SEQ ID NO: 5526 (700357108H1), SEQ ID NO: 5529 (700357122H1), SEQ ID NO: 5371 (700356474H1), SEQ ID NO: 4029 (700353005H1), SEQ ID NO: 5099 (700355779H1), SEQ ID NO: 4100 (700353128H1), SEQ ID NO: 3790 (700282575H2), SEQ ID NO: 3835 (700352675H1), SEQ ID NO: 3943 (700352843H1), SEQ ID NO: 3856 (700352709H1), SEQ ID NO: 3874 (700352735H1), SEQ ID NO: 4959 (700355462H1), SEQ ID NO: 5010 (700355558H1), SEQ ID NO: 4336 (700353828H1), SEQ ID NO: 5420 (700356627H1), SEQ ID NO: 5751 (700382275H1), SEQ ID NO: 5301 (700356219H1), SEQ ID NO: 6184 (700383158H1), SEQ ID NO: 5003 (700355547H1), SEQ ID NO: 4800 (700355026H1), SEQ ID NO: 4610 (700354647H1), SEQ ID NO: 4789 (700355009H1), SEQ ID NO: 4466 (700354337H1), SEQ ID NO: 5278 (700356158H1), SEQ ID NO: 4742 (700354908H1), SEQ ID NO: 5260 (700356129H1), SEQ ID NO: 5661 (700382106H1), SEQ ID NO: 3806 (700352616H1), SEQ ID NO: 4555 (700354518H1), SEQ ID NO: 5232 (700356066H1), SEQ ID NO: 5216 (700356023H1), SEQ ID NO: 6002 (700382827H1), SEQ ID NO: 4217 (700353446H1), SEQ ID NO: 4793 (700355016H1), SEQ ID NO: 4204 (700353409H1), SEQ ID NO: 6178 (700383149H1), SEQ ID NO: 6083 (700382979H1), SEQ ID NO: 4876 (700355249H1), SEQ ID NO: 4031 (700353007H1), SEQ ID NO: 6098 (700383013H1), SEQ ID NO: 6102 (700383021H1), SEQ ID NO: 4421 (700354239H1), SEQ ID NO: 5946 (700382708H1), SEQ ID NO: 5214 (700356()20H1), SEQ ID NO: 5703 (700382195H1), SEQ ID NO: 3750 (700282503H2), SEQ ID NO: 4212 (700353432H1), SEQ ID NO: 6090 (700382995H1), SEQ ID NO: 4713 (700354850H1), SEQ ID NO: 4197 (700353284H1), SEQ ID NO: 5692 (700382179H1), SEQ ID NO: 4727 (700354875H1), SEQ ID NO: 5924 (700382668H1), SEQ ID NO: 4052 (700353052H1), SEQ ID NO: 4111 (700353142H1), SEQ ID NO: 4043 (700353028H1), SEQ ID NO: 5723 (700382229H1), SEQ ID NO: 5560 (700357402H1), SEQ ID NO: 5603 (700357489H1), SEQ ID NO: 5700 (700382191H1), SEQ ID NO: 5875 (700382543H1), SEQ ID NO: 3811 (700352625H1), SEQ ID NO: 6081 (700382975H1), SEQ ID NO: 5186 (700355963H1), SEQ ID NO: 5813 (700382392H1), SEQ ID NO: 5155 (700355889H1), SEQ ID NO: 4632 (700354689H1), SEQ ID NO: 5674 (700382139H1), SEQ ID NO: 4279 (700353629H1), SEQ ID NO: 4625 (700354676H1), SEQ ID NO: 5586 (700357445H1), SEQ ID NO: 4054 (700353057H1), SEQ ID NO: 5724 (700382230H1), SEQ ID NO: 5699 (700382190H1), SEQ ID NO: 6181 (700383154H1), SEQ ID NO: 3982 (700352912H1), SEQ ID NO: 4654 (700354742H1), SEQ ID NO: 4737 (700354895H1), SEQ ID NO: 4321 (700353779H1), SEQ ID NO: 4934 (700355415H1), SEQ ID NO: 5543 (700357160H1), SEQ ID NO: 4933 (700355414H1), SEQ ID NO: 3713 (700282435H1), SEQ ID NO: 3787 (700282569H2), SEQ ID NO: 3789 (700282571H2), SEQ ID NO: 4814 (700355053H1), SEQ ID NO: 5555 (700357189H1), SEQ ID NO: 3876 (700352737H1), SEQ ID NO: 4208 (700353424H1), SEQ ID NO: 3827 (700352654H1), SEQ ID NO: 3863 (700352719H1), SEQ ID NO: 6130 (700383062H1), SEQ ID NO: 4041 (700353023H1), SEQ ID NO: 5101 (700355781H1), SEQ ID NO: 5792 (700382353H1), SEQ ID NO: 4983 (700355513H1), SEQ ID NO: 4089 (700353114H1), SEQ ID NO: 4348 (700353853H1), SEQ ID NO: 6182 (700383155H1), SEQ ID NO: 5716 (700382218H1), SEQ ID NO: 5102 (700355783H1), SEQ ID NO: 5859 (700382507H1), SEQ ID NO: 5442 (700356725H1), SEQ ID NO: 5998 (700382817H1), SEQ ID NO: 4037 (700353014H1), SEQ ID NO: 4404 (700354142H1), SEQ ID NO: 5734 (700382245H1), SEQ ID NO: 5392 (700356534H1), SEQ ID NO: 5675 (700382140H1), SEQ ID NO: 5746 (700382267H1), SEQ ID NO: 4102 (700353130H1), SEQ ID NO: 4536 (700354478H1), SEQ ID NO: 4219 (700353509H1), SEQ ID NO: 5151 (700355885H1), SEQ ID NO: 4268 (700353612H1), SEQ ID NO: 3774 (700282549H2), SEQ ID NO: 3772 (700282547H2), SEQ ID NO: 5283 (700356171H1), SEQ ID NO: 5647 (700382075H1), SEQ ID NO: 5778 (700382330H1), SEQ ID NO: 5014 (700355577H1), SEQ ID ND: 5825 (700382426H1), SEQ ID NO: 5866 (700382525H1), SEQ ID NO: 4556 (700354519H1), SEQ ID NO: 3821 (700352645H1), SEQ ID NO: 4762 (700354942H1), SEQ ID NO: 4981 (700355507H1), SEQ ID NO: 5540 (700357155H1), SEQ ID NO: 5476 (700356835H1), SEQ ID NO: 5650 (700382078H1), SEQ ID NO: 5750 (700382273H1), SEQ ID NO: 5100 (700355780H1), SEQ ID NO: 4553 (700354516H1), SEQ ID NO: 4923 (700355345H1), SEQ ID NO: 4922 (700355344H1), SEQ ID NO: 3773 (700282548H2), SEQ ID NO: 3715 (700282438H2), SEQ ID NO: 5022 (700355594H1), SEQ ID NO: 5516 (700356974H1), SEQ ID NO: 4451 (700354305H1), SEQ ID NO: 4938 (700355422H1), SEQ ID NO: 5436 (700356695H1), SEQ ID NO: 5742 (700382260H1), SEQ ID NO: 5242 (700356081H1), SEQ ID NO: 4766 (700354954H1), SEQ ID NO: 3792 (700282584H2), SEQ ID NO: 3795 (700282592H2), SEQ ID NO: 5569 (700357415H1), SEQ ID NO: 5142 (700355871H11), SEQ ID NO: 4305 (700353713H1), SEQ ID NO: 4559 (700354534H1), SEQ ID NO: 5188 (700355966H1), SEQ ID NO: 5088 (700355766H1), SEQ ID NO: 5286 (700356177H1), SEQ ID NO: 5294 (700356193H1), SEQ ID NO: 3995 (700352935H1), SEQ ID NO: 4615 (700354658H1), SEQ ID NO: 5453 (700356767H1), SEQ ID NO: 4667 (700354769H1), SEQ ID NO: 5832 (700382439H1), SEQ ID NO: 5839 (700382450H1), SEQ ID NO: 6101 (700383019H1), SEQ ID NO: 6097 (700383011H1), SEQ ID NO: 4951 (700355447H1), SEQ ID NO: 4423 (700354247H1), SEQ ID NO: 4672 (700354777H1), SEQ ID NO: 4682 (700354793H1), SEQ ID NO: 5395 (700356541H1), SEQ ID NO: 5311 (700356241H1), SEQ ID NO: 4522 (700354446H1), SEQ ID NO: 4460 (700354326H1), SEQ ID NO: 5493 (700356901H1), SEQ ID NO: 5507 (700356933H1), SEQ ID NO: 4512 (700354425H1), SEQ ID NO: 5217 (700356025H1), SEQ ID NO: 4159 (700353215H1), SEQ ID NO: 5480 (700356847H1), SEQ ID NO: 6123 (700383051H1), SEQ ID NO: 4452 (700354310H1), SEQ ID NO: 4499 (700354409H1), SEQ ID NO: 4808 (700355040H1), SEQ ID NO: 5800 (700382365H1), SEQ ID NO: 5710 (700382210H1), SEQ ID NO: 6030 (700382882H1), SEQ ID NO: 5461 (700356796H1), SEQ ID NO: 5548 (700357173H1), SEQ ID NO: 4790 (700355011H1), SEQ ID NO: 5201 (700355989H1), SEQ ID NO: 4286 (700353653H1), SEQ ID NO: 6103 (700383023H1), SEQ ID NO: 6099 (700383015H1), SEQ ID NO: 5317 (700356252H1), SEQ ID NO: 5399 (700356551H1), SEQ ID NO: 5667 (700382123H1), SEQ ID NO: 6176 (700383147H1), SEQ ID NO: 5084 (700355758H1), SEQ ID NO: 4566 (700354558H1), SEQ ID NO: 4845 (700355173H1), SEQ ID NO: 4819 (700355067H1), SEQ ID NO: 3775 (700282550H2), SEQ ID NO: 5273 (700356152H1), SEQ ID NO: 6111 (700383037H1), SEQ ID NO: 3984 (700352916H1), SEQ ID NO: 5481 (700356848H1), SEQ ID NO: 5309 (700356236H1), SEQ ID NO: 4456 (700354317H1), SEQ ID NO: 4824 (700355077H1), SEQ ID NO: 5240 (700356076H1), SEQ ID NO: 5121 (700355829H1), SEQ ID NO: 5418 (700356624H1), SEQ ID NO: 4603 (700354637H1), SEQ ID ND: 5718 (700382220H1), SEQ ID NO: 5747 (700382268H1), SEQ ID NO: 6189 (700383166H1), SEQ ID NO: 6129 (700383061H1), SEQ ID NO: 5494 (700356903H1), SEQ ID ND: 5508 (700356935H1), SEQ ID NO: 4797 (700355022H1), SEQ ID NO: 5691 (700382176H1), SEQ ID NO: 5862 (700382513H1), SEQ ID NO: 5917 (700382654H1), SEQ ID NO: 5328 (700356273H1), SEQ ID NO: 3971 (700352891H1), SEQ ID NO: 5463 (700356802H1), SEQ ID NO: 4588 (700354606H1), SEQ ID NO: 4342 (700353839H1), SEQ ID NO: 5376 (700356483H1), SEQ ID NO: 5136 (700355856H1), SEQ ID NO: 5154 (7003558,38H1), SEQ ID NO: 5916 (700382649H1), SEQ ID NO: 5869 (700382537H1), SEQ ID NO: 5231 (700356062H1), SEQ ID NO: 4194 (700353279H1), SEQ ID NO: 4978 (700355496H1), SEQ ID NO: 5923 (700382665H1), SEQ ID NO: 4755 (700354930H1), SEQ ID NO: 4008 (700352961H1), SEQ ID NO: 5424 (700356644H1), SEQ ID NO: 4035 (700353012H1), SEQ ID NO: 5323 (700356265H1), SEQ ID NO: 4818 (700355065H1), SEQ ID NO: 5367 (700356468H1), SEQ ID NO: 5888 (700382579H1), SEQ ID NO: 3690 (7002824(1H2), SEQ ID NO: 3794 (700282591H2), SEQ ID NO: 3764 (700282522H2), SEQ ID NO: 3758 (700282514H2), SEQ ID NO: 5128 (700355842H1), SEQ ID NO: 3818 (7003526.36H1), SEQ ID NO: 3781 (700282561H2), SEQ ID NO: 3782 (700282562H2), SEQ ID NO: 4889 (700355286H1), SEQ ID NO: 6104 (700383026H1), SEQ ID NO: 5138 (700355861H11), SEQ ID NO: 5140 (700355866H1), SEQ ID NO: 6006 (700382834H1), SEQ ID 1NO: 5880 (700382554H1), SEQ ID NO: 6116 (700383043H1), SEQ ID NO: 5774 (700382324H1), SEQ ID NO: 4024 (700352993H1), SEQ ID NO: 4820 (7003550701), SEQ ID NO: 5438 (700356702H1), SEQ ID NO: 4150 (700353201H1), SEQ ID NO: 5909 (700382636H1), SEQ ID NO: 5551 (700357183H1), SEQ ID NO: 5055 (700355708H1), SEQ ID NO: 4551 (700354508H1), SEQ ID NO: 3975 (700352903H1), SEQ ID NO: 5793 (700382354H1), SEQ ID NO: 5823 (700382424H1), SEQ ID NO: 3915 (700352802H1), SEQ ID NO: 5146 (700355877H1), SEQ ID NO: 5879 (700382550H1), SEQ ID NO: 5627 (700382041l1), SEQ ID NO: 4211 (700353431H1), SEQ ID NO: 5894 (700382604H1), SEQ ID NO: 5806 (700382377H1), SEQ ID NO: 5558 (700357194H1), SEQ ID NO: 5396 (700356542H1), SEQ ID NO: 3892 (700352760H1), SEQ ID NO: 5211 (700356015H1), SEQ ID NO: 4011 (700352967H1), SEQ ID NO: 5282 (700356167H1), SEQ ID NO: 5978 (700382173H1), SEQ ID NO: 4898 (700355307H1), SEQ ID NO: 5522 (700356984H1), SEQ ID NO: 5226 (700356047H1), SEQ ID NO: 3777 (700282554H2), SEQ ID NO: 3776 (700282553H2), SEQ ID NO: 5817 (700382401H1), SEQ ID NO: 5858 (700382494H1), SEQ ID NO: 4627 (700354678H1), SEQ ID NO: 5411 (700356591H1), SEQ ID NO: 4257 (700353590H1), SEQ ID NO: 4833 (700355093H1), SEQ ID NO: 4148 (700353195H1), SEQ ID NO: 5210 (700356014H1), SEQ ID NO: 4422 (700354244H1), SEQ ID NO: 4949 (700355444H1), SEQ ID NO: 4481 (700354371H1), SEQ ID NO: 6095 (700383009H1), SEQ ID NO: 4670 (700354774H1), SEQ ID NO: 4680 (700354790H1). These selected cdps represent unique, corn tassel-specific polynucleotides which are used to produce a tassel-specific profile of gene transcription, a transcript image.

The cdps are also used as a composition in methods to detect altered gene expression in inbred or hybrid plants. Such methods employ the cdps of the Sequence Listing, oligonucleotides, fragments, derivatives, or complementary sequences in hybridization technologies. The invention provides a method for detecting polynucleotides in a biological sample, the method comprising the steps of hybridizing a cdp to at least one of the nucleic acids in the biological sample, thereby forming a hybridization complex, and detecting the hybridization complex, wherein the presence of the complex correlates with the presence of the polynucleotide in the sample. An additional method provides for amplification of the nucleic acids of the biological sample prior to hybridization. The invention provides a method of screening a plurality of molecules for specific binding to a polynucleotide, the method comprising the steps of providing the plurality of molecules; combining the polynucleotide with each of the plurality of molecules for a time sufficient to allow binding under suitable conditions; and detecting binding of the polynucleotide to each of the plurality of molecules, thereby identifying the molecules which specifically bind the polynucleotide.

The invention further provides a method for recovering a regulatory element, the method comprising the steps of designing oligomers to at least one of the cdps, combining the oligomer, with a DNA library under appropriate conditions to amplify the cdp, comparing the cdp with the amplified sequence to identify overlapping areas, identifying additional sequence beyond the overlapping areas, and repeating steps a) through d) until the regulatory element is recovered. The regulatory element is a tassel-specific regulatory element which may be placed in an expression vector which is transformed into a corn plant. The regulatory element is of value in regulating the expression of introduced cdps.

The invention provides a purified corn tassel-derived polynucleotide capable of expressing a corn tassel-derived polypeptide. In one embodiment, the corn tassel-derived polynucleotide is contained within an expression vector. In a second embodiment, the expression vector is contained within a host cell. The invention also provides a method for producing a corn tassel-derived polypeptide, said method comprising the steps of culturing the host cells containing the tassel-derived polynucleotide under conditions suitable for the expression of a corn tassel-derived polypeptide, and recovering the corn tassel-derived polypeptide from the cell culture.

The invention provides a purified corn tassel-derived polypeptide (CDP) encoded by at least one of the cdps of the Sequence Listing. The invention also provides an anti-CDP antibody specific for a purified polypeptide encoded by the cdp. Such antibodies may be used for diagnostic purposes for the detection of CDPs in specific plant cells.

The invention further provides a method for identifying a test compound which specifically binds the CDP, the method comprising the steps of providing a test compound, combining the CDP with the test compound under suitable conditions for a time sufficient to allow binding, and detecting CDP binding to the test compound.

### DESCRIPTION OF THE SEQUENCE LISTING AND TABLES

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

The Sequence Listing is a compilation of nucleic acid sequences obtained by sequencing clone inserts or isolates of two corn tassel cDNA libraries. Each sequence is identified by a sequence identification number (SEQ ID NO) and by an Incyte Clone number. TABLES 1 and 2 are compilations of Incyte Clones arranged as described below.

TABLE 1 lists homologous isolates from the two corn tassel cDNA libraries prepared as described in the Examples. The first column contains Incyte Clone numbers. The Incyte Clone number provides a cross reference to the Sequence Listing. The second column contains a relevant GenBank identification number. The third and fourth columns represent product and log-likelihood scores (Karlin, S. and S. F. Altschul (1993) Proc. Nat. Acad. Sci. 90:5873-5877). The fifth column refers to the particular database and release of GenBank against which the Incyte Clone was searched and in which a related sequence was found. The sequences of this invention were compared to sequences in the GenBank plant, eukaryotic and protein databases, and most isolates list homology to sequences in those databases. The last column contains a description of the referenced GenBank sequence.

TABLE 2 is a compilation representing corn tassel-specific gene activity as illustrated by sets of clustered or related sequences. Each cluster disclosed in the table contains unique or homologous sequences that are specific to the two corn tassel cDNA libraries. The clones in a cluster may contain overlapping sequences or they may be related to or overlap a common reference sequence. Clusters are compiled by naming, matching, and counting all copies of the cdp. The minimum number of clones required to define a cluster is two; clusters containing two clones are found at the bottom of the table. Some clusters are characterized further by the homology of one or more sequences to a reference sequence which has a GenBank identifier (g) and description. Homologous sequences are more fully described in TABLE 1.

### DETAILED DESCRIPTION OF THE INVENTION

Before the nucleic acid sequences and methods are presented, it is to be understood that this invention is not limited to the particular methodologies, protocols, cell lines, vectors, and reagents described. Although particular embodiments are described, methods and materials similar or equivalent to these embodiments may be used to practice the invention. The preferred methods, devices, and materials set forth are not intended to limit the scope of the invention which is limited only by the appended claims.

The singular forms “a”, “an”, and “the” include plural reference unless the context clearly dictates otherwise. All technical and scientific terms have the meanings commonly understood by one of ordinary skill in the art. All publications are incorporated by reference for the purpose of describing and disclosing the cell lines, vectors, and methodologies which are presented and which might be used in connection with the invention. Nothing in the specification is to be construed as an admission that the invention is not entitled to antedate such disclosure by virtue of prior invention.

Definitions

As used herein, the lower case “cdp” refers to a nucleic acid sequence, while the upper case “CDP” refers to an amino acid sequence. A “full-length” cdp refers to a nucleic acid sequence containing the entire coding region of a gene endogenously expressed in corn.

“Adjuvants” are materials such as Freund's, mineral gels (aluminum hydroxide), and surface active substances (lysolecithin, pluronic polyols, polyanions, peptides, oil emulsions, keyhole limpet hemocyanin (KLH; Sigma-Aldrich, St. Louis Mo.), and dinitrophenol) which may be administered to increase a host's immunological response.

“Allele” refers to an alternative form of a nucleic acid sequence. Alleles result from a “mutation”, and any given genome may have none, one, or many allelic forms. Mutations which give rise to alleles are ascribed to deletions, additions or substitutions of nucleotides. Each of these types of changes may occur alone, or in combination with the others, one or more times in a given nucleic acid sequence. The present invention encompasses allelic cdps.

“Amino acid sequence” refers to an oligopeptide, a peptide, a polypeptide, or a protein of either natural or synthetic origin. The amino acid sequence is not limited to the complete, endogenous amino acid sequence and may be a portion, epitope, variant, or derivative of a protein expressed by a corn nucleic acid sequence.

“Amplification” refers to the production of additional copies of a sequence and is carried out using polymerase chain reaction (PCR) technologies well known in the art.

“Antisense” refers to nucleic acid sequences which are complementary to a specific DNA or RNA sequence. The antisense strand (negative or 3′-5′) is that nucleic acid strand that is complementary to the sense strand (positive or 5′-3′).

“Biologically active” refers to a peptide having a structural, regulatory biochemical, or immunological function of a naturally occurring peptide.

“Complementary” refers to the natural association of nucleic acid sequences by base-pairing (A-G-T pairs with its complement T-C-A). Complementarity between two single-stranded molecules may be partial or complete. The degree to which two sequences are complementary affects the efficiency of hybridization and amplification reactions.

“Derivative” refers to the chemical modification of a nucleic acid sequence by replacement of hydrogen by an alkyl, acyl, amino, or other group.

“Homology” refers to sequence similarity either between a reference nucleic acid sequence and at least a fragment of a cdp or between a reference amino acid sequence and a portion of a CDP.

“Hybridization” refers to the process by which a strand of nucleic acid joins with a complementary strand through base pairing.

“Immunogenic” defines the capability of a natural, recombinant or synthetic oligopeptide, or polypeptide, to induce antibody production in appropriate animals or cells.

“Labeling” refers to the covalent or noncovalent joining of a polynucleotide, polypeptide or antibody with a reporter molecule that provides for a detectable and often measurable signal.

“Linkers” are short stretches of nucleotide sequence which may be added onto a vector or a cdp to create restriction endonuclease sites for ease in cloning. “Polylinkers” are engineered to include multiple restriction enzyme sites and provide for the use of both those enzymes which leave 5′ and 3′ overhangs (such as BamHI, EcoRI, and HindIII) or which provide a blunt end (such as EcoRV, SnaBI, and StuI).

“Naturally occurring” refers to an endogenous polynucleotide or polypeptide that may be isolated from viruses or prokaryotic or eukaryotic cells.

“Nucleic acid sequence” refers to an oligomer, oligonucleotide, nucleotide or polynucleotide, and its fragments, and to DNA or RNA of genomic or synthetic origin which may be single- or double-stranded and represent the sense or complementary (antisense) strand.

“Oligomer” refers to a nucleic acid sequence of at least about 10 nucleotides and as many as about 60 nucleotides, preferably about 15 to 30 nucleotides, that may be used as a primer or amplimer in amplification technologies. Oligomers are usually chemically synthesized.

“Peptide nucleic acid” (PNA) refers to an oligomer of at least six nucleotides to which an amino acid residue, such as lysine, and an amino group have been added. PNAs, also designated antigene agents, stop transcript elongation by binding to their complementary strand of nucleic acid.

“Plant sample” refers to a cell, chromosomes isolated from a cell, genomic DNA, RNA, or cDNA in solution or bound to a substrate; an extract from plant cells, a cleared tissue, a blot or imprint from the cut edge of a plant part, or the like.

A “portion” of an CDP may be selected based upon retention of biological or immunological characteristics shared with naturally occurring polypeptides derived from corn tassel. For example, an antigenic portion of a CDP may be used to induce antibody in an appropriate host.

“Post-translational modification” of a CDP may involve lipidation, glycosylation, phosphorylation, acetylation, racemization, proteolytic cleavage, and the like. These processes may occur synthetically or biochemically. Biochemical modifications will vary by cell type depending on the enzymatic milieu and the CDP.

“Probe” refers to cdps or fragments thereof, which are used to detect identical, allelic or related nucleic acid sequences.

“Purified” refers to molecules, either polynucleotides or polypeptides that are isolated or separated from their natural environment and are at least 60% free, preferably 75% free, and most preferably 90% free from other compounds with which they are naturally associated.

“Regulatory element” refers to a nucleic acid sequence from nontranslated regions of a gene such as enhancers, promoters, introns, and 3′ untranslated regions which interact with host proteins to carry out transcription or translation.

“Reporter” molecules are chemical or biochemical moieties used for labeling a nucleic acid, an amino acid, or an antibody. They include radionuclides; enzymes; fluorescent, chemiluminescent, or chromogenic agents; substrates; cofactors; inhibitors; magnetic particles; and the like.

“Substrate” refers to any suitable rigid or semi-rigid support including membranes, filters, chips, slides, wafers, fibers, magnetic or nonmagnetic beads, gels, capillaries or other tubing, plates, polymers, and microparticles. The substrate can have a variety of surface forms, such as wells, trenches, pins, channels and pores, to which the polynucleotides are bound.

“Transformation” refers to a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed. Transformants include stably transformed cells in which the inserted DNA is capable of replication either as an autonomously replicating plasmid or as part of the host chromosome, as well as cells which transiently express the inserted DNA or RNA.

“Variant” refers to an amino acid sequence which differs from another sequence by at least one amino acid. The variant may have “conservative” changes (e.g. replacement of leucine with isoleucine), which does not affect structural or chemical properties; or more rarely, “nonconservative” changes (e.g. replacement of glycine with tryptophan), which may affect structural and/or chemical properties.

### THE INVENTION

In a particular embodiment, mRNA was isolated from corn tassel and used to construct the SATMON021 and SATMON024 cDNA libraries. The invention relates to nucleic acid sequences comprising corn tassel-derived polynucleotides (cdps) as presented in the Sequence Listing and to the use of these nucleic acid sequences. A “corn tassel-derived polynucleotide” refers to a cdp which may be naturally occurring, recombinant, synthetic, or semi-synthetic. A subset of clustered tassel-specific cdps is given in TABLE 2. The cdps may be used to identify, isolate, or extend identical or related corn tassel nucleic acid sequences from DNA libraries for the purpose of producing an entire coding region or recovering a regulatory element. The cdps may also be used in nucleic acid hybridization cr amplification technologies to follow expression of desirable traits through plant breeding programs. The present invention provides for expression vectors and host cells containing nucleic acid sequences that encode CDPs or portions thereof and regulatory elements obtained using methods described herein. The CDPs may possess biological or immunological activity, or both. The invention provides for the use of purified CDPs to induce antibodies for diagnostic use and to identify test compounds which specifically bind the CDP.

Specifically, the present invention relates to the following subset of unique and tassel-specific cdps whose transcripts occurred more than once in thetwo corn tassel cDNA libraries. They are SEQ ID NO: 1615 (700344704H1), SEQ ID NO: 2691 (700346502H1), SEQ ID NO: 2813 (700346702H1), SEQ ID NO: 3616 (700380601H1), SEQ ID NO: 3681 (700380774H1), SEQ ID NO: 233 (700342388H1), SEQ ID NO: 2060 (700345470H1), SEQ ID NO: 964 (700343568H1), SEQ ID NO: 2811 (700346695H1), SEQ ID NO: 2519 (700346233H1), SEQ ID NO: 2083 (70034551051), SEQ ID NO: 1101 (70034378051), SEQ ID NO: 1847 (700345052H1), SEQ ID NO: 1777 (700344946H1), SEQ ID NO: 1780 (700344949H1), SEQ ID NO: 1733 (700344870H1), SEQ ID NO: 3018 (700347036H1), SEQ ID NO: 3158 (700347281H1), SEQ ID NO: 2220 (700345749H1), SEQ ID NO: 165 (700342268H1), SEQ ID NO: 139 (700342223H1), SEQ ID NO: 2823 (700346721H1), SEQ ID NO: 2065 (700345477H1), SEQ ID NO: 605 (700343040H1), SEQ ID NO: 3618 (700380603H1), SEQ ID NO: 1537 (700344525H1), SEQ ID NO: 469 (700342790H1), SEQ ID NO: 3344 (700379867H1), SEQ ID NO: 180 (700342291H1), SEQ ID NO: 303 (700342521H1), SEQ ID NO: 1249 (700344013H1), SEQ ID NO: 1036 (700343679H1), SEQ ID NO: 2920 (700346877H1), SEQ ID NO: 3619 (700380604H1), SEQ ID NO: 1061 (700343721H1), SEQ ID NO: 1589 (700344654H1), SEQ ID NO: 3100 (700347193H1), SEQ ID NO: 2182 (700345675H1), SEQ ID NO: 2218 (700345743H1), SEQ ID NO: 398 (700342679H1), SEQ ID NO: 175 (700342283H1), SEQ ID NO: 3533 (700380411H1), SEQ ID NO: 3615 (700380596H1), SEQ ID NO: 89 (700281941H2), SEQ ID NO: 3530 (700380405H1), SEQ ID NO: 3023 (700347043H1), SEQ ID NO: 3185 (700347339H1), SEQ ID NO: 3062 (700347112H1), SEQ ID NO: 1082 (700343752H1), SEQ ID NO: 55 (700281888H2), SEQ ID NO: 2955 (700346930H1), SEQ ID NO: 1032 (700343674H1), SEQ ID NO: 163 (700342263H1), SEQ ID NO: 2864 (700346807H1), SEQ ID NO: 1286 (700344069H1), SEQ ID NO: 2560 (700346291H1), SEQ ID NO: 742 (700343247H1), SEQ ID NO: 1731 (700344866H1), SEQ ID NO: 370 (700342630H1), SEQ ID NO: 1167 (700343872H1), SEQ ID NO: 715 (700343202H1), SEQ ID NO: 3314 (700379793H1), SEQ ID NO: 2156 (700345638H1), SEQ ID NO: 624 (700343071H1), SEQ ID NO: 545 (700342924H1), SEQ ID NO: 2855 (700346794H1), SEQ ID NO: 850 (700343408H1), SEQ ID NO: 1557 (700344614H1), SEQ ID NO: 1872 (700345114H1), SEQ ID NO: 1000 (700343626H1), SEQ ID NO: 1045 (700343695H1), SEQ ID NO: 2753 (700346604H1), SEQ ID NO: 70 (700281910H1), SEQ ID NO: 1235 (700343988H1), SEQ ID NO: 389 (700342664H1), SEQ ID NO: 486 (700342817H1), SEQ ID NO: 766 (700343281H1), SEQ ID NO: 1310 (700344105H1), SEQ ID NO: 3202 (700347368H1), SEQ ID NO: 1941 (700345253H1), SEQ ID NO: 6 (700281814H2), SEQ ID NO: 1884 (700345138H1), SEQ ID NO: 2316 (700345916H1), SEQ ID NO: 1948 (700345263H1), SEQ ID NO: 2277 (700345849H1), SEQ ID NO: 2019 (700345394H1), SEQ ID NO: 1696 (700344820H1), SEQ ID NO: 226 (700342373H1), SEQ ID NO: 262 (700342448H1), SEQ ID NO: 1377 (700344208H1), SEQ ID NO: 1279 (700344059H1), SEQ ID NO: 1770 (700344935H1), SEQ ID NO: 532 (700342903H1), SEQ ID NO: 843 (700343396H1), SEQ ID NO: 360 (700342616H1), SEQ ID NO: 540 (700342915H1), SEQ ID NO: 413 (700342708H1), SEQ ID NO: 1252 (700344017H1), SEQ ID NO: 1560 (700344618H1), SEQ ID NO: 3541 (700380432H1), SEQ ID NO: 243 (700342413H1), SEQ ID NO: 1938 (700345249H1), SEQ ID NO: 1127 (700343816H1), SEQ ID NO: 1545 (700344541H1), SEQ ID NO: 1946 (700345258H1), SEQ ID NO: 298 (700342510H1), SEQ ID NO: 2906 (700346860H1), SEQ ID NO: 783 (700343310H1), SEQ ID NO: 616 (700343060H1), SEQ ID NO: 2097 (700345529H1), SEQ ID NO: 2393 (700346036H1), SEQ ID NO: 860 (700343420H1), SEQ ID NO: 2196 (700345705H1), SEQ ID NO: 2403 (700346049H1), SEQ ID NO: 2309 (700345906H1), SEQ ID NO: 1963 (700345289H1), SEQ ID NO: 1383 (700344218H1), SEQ ID NO: 3201 (700347367H1), SEQ ID NO: 3589 (700380530H1), SEQ ID NO: 660 (7003431241H1), SEQ ID NO: 2254 (700345807H1), SEQ ID NO: 2689 (700346496H1), SEQ ID NO: 3166 (700347296H1), SEQ ID NO: 2023 (700345404H1), SEQ ID NO: 726 (700343218H1), SEQ ID NO: 363 (700342620H1), SEQ ID NO: 2968 (700346951H1), SEQ ID NO: 2214 (700345737H1), SEQ ID NO: 989 (700343607H1), SEQ ID NO: 2642 (700346426H1), SEQ ID NO: 2741 (700346581H1), SEQ ID NO: 1321 (700344122H1), SEQ ID NO: 1255 (700344022H1), SEQ ID NO: 2311 (700345908H1), SEQ ID NO: 1857 (700345075H1), SEQ ID NO: 2571 (700346316H1), SEQ ID NO: 3063 (700347116H1), SEQ ID NO: 1649 (700344750H1), SEQ ID NO: 1653 (700344754H1), SEQ ID NO: 1762 (700344924H1), SEQ ID NO: 1985 (700345329H1), SEQ ID NO: 2382 (700346023H1), SEQ ID NO: 2468 (700346152H1), SEQ ID NO: 1567 (700344628H1), SEQ ID NO: 991 (700343609H1), SEQ ID NO: 2124 (700345577H1), SEQ ID NO: 829 (700343377H1), SEQ ID NC: 3623 (700380616H1), SEQ ID NO: 725 (700343216H1), SEQ ID NO: 1676 (700344785H1), SEQ ID NO: 399 (700342680H1), SEQ ID NO: 2790 (700346658H1), SEQ ID NO: 2284 (700345859H1), SEQ ID NO: 2838 (700346745H1), SEQ ID NO: 757 (700343266H1), SEQ ID NO: 1251 (700344016H1), SEQ ID NO: 207 (700342338H1), SEQ ID NC: 3515 (700380270H1), SEQ ID NO: 3224 (700379610H1), SEQ ID NO: 2958 (700346935H1), SEQ ID NO: 1861 (700345079H1), SEQ ID NO: 3052 (700347092H1), SEQ ID NO: 2542 (700346264H1), SEQ ID NO: 3602 (700380571H1), SEQ ID NO: 2788 (700346656H1), SEQ ID NO: 1966 (700345293H1), SEQ ID NO: 1813 (700345004H1), SEQ ID NO: 573 (700342986H1), SEQ ID NO: 3572 (700380488H1), SEQ ID NO: 1368 (700344191H1), SEQ ID NO: 774 (700343291H1), SEQ ID NO: 443 (700342747H1), SEQ ID NO: 1083 (700343755H1), SEQ ID NO: 110 (700281973H1), SEQ ID NO: 118 (700281986H2), SEQ ID NO: 2088 (700345516H1), SEQ ID NO: 2932 (700346894H1), SEQ ID NO: 2815 (700346704H1), SEQ ID NO: 1112 (700343793H1), SEQ ID NO: 3131 (700347236H1), SEQ ID NO: 682 (700343154H1), SEQ ID NO: 1965 (700345291H1), SEQ ID NO: 1866 (700345094H1), SEQ ID NO: 898 (700343475H1), SEQ ID NO: 3582 (700380514H1), SEQ ID NO: 916 (700343509H1), SEQ ID NO: 188 (700342309H1), SEQ ID NO: 1364 (700344186H1), SEQ ID NO: 1421 (700344295H1), SEQ ID NO: 3685 (700380786H1), SEQ ID NO: 2404 (700346050H1), SEQ ID NO: 3246 (700379650H1), SEQ ID NO: 1673 (700344782H1), SEQ ID NO: 953 (700343552H1), SEQ ID NO: 2216 (700345739H1), SEQ ID NO: 1232 (700343985H1), SEQ ID NO: 775 (700343292H1), SEQ ID NO: 869 (700343431H1), SEQ ID NO: 145 (700342231H1), SEQ ID NO: 2927 (700346887H1), SEQ ID NO: 1820 (700345013H1), SEQ ID NO: 2244 (700345785H1), SEQ ID NO: 1078 (700343746H1), SEQ ID NO: 2965 (700346943H1), SEQ ID NO: 1085 (700343757H1), SEQ ID NO: 730 (700343225H1), SEQ ID NO: 792 (700343325H1), SEQ ID NO: 3218 (700347395H1), SEQ ID NO: 3033 (700347064H1), SEQ ID NO: 1058 (700343715H1), SEQ ID NO: 302 (700342515H1), SEQ ID NO: 2384 (700346026H1), SEQ ID NO: 1220 (700343965H1), SEQ ID NO: 1883 (700345136H1), SEQ ID NO: 1928 (700345235H1), SEQ ID NO: 2598 (700346351H1), SEQ ID NO: 315 (700342543H1), SEQ ID NO: 1405 (700344265H1), SEQ ID NO: 3128 (700347232H1), SEQ ID NO: 942 (700343540H1), SEQ ID NO: 1109 (700343790H1), SEQ ID NO: 1490 (700344425H1), SEQ ID NO: 3040 (700347072H1), SEQ ID NO: 2411 (700346060H1), SEQ ID NO: 2477 (700346167H1), SEQ ID NO: 446 (700342755H1), SEQ ID NO: 3026 (700347050H1), SEQ ID NO: 3554 (700380452H1), SEQ ID NO: 3465 (700380146H1), SEQ ID NO: 143 (700342228H1), SEQ ID NO: 867 (700343428H1), SEQ ID NO: 1139 (700343832H1), SEQ ID NO: 3067 (700347126H1), SEQ ID NO: 799 (700343333H1), SEQ ID NO: 2059 (700345469H1), SEQ ID NO: 2120 (700345571H1), SEQ ID NO: 2179 (700345671H1), SEQ ID NO: 2707 (700346525H1), SEQ ID NO: 967 (700343572H1), SEQ ID NO: 1356 (700344174H1), SEQ ID NO: 2896 (700346848H1), SEQ ID NO: 304 (700342523H1), SEQ ID NO: 1063 (700343723H1), SEQ ID NO: 560 (700342959H1), SEQ ID NO: 2625 (700346389H1), SEQ ID NO: 2501 (700346210H1), SEQ ID NO: 319 (700342551H1), SEQ ID NO: 3173 (700347308H1), SEQ ID NO: 2313 (700345910H1), SEQ ID NO: 2022 (700345403H1), SEQ ID NO: 1076 (700343742H1), SEQ ID NO: 3025 (700347049H1), SEQ ID NO: 1719 (700344849H1), SEQ ID NO: 3342 (700379862H1), SEQ ID NO: 643 (7003431C3H1), SEQ ID NO: 3649 (700380682H1), SEQ ID NO: 3613 (700380589H1), SEQ ID NO: 807 (700343342H1), SEQ ID NO: 1760 (700344920H1), SEQ ID NO: 2558 (700346288H1), SEQ ID NO: 492 (700342832H1), SEQ ID NO: 504 (700342846H1), SEQ ID NO: 172 (700342278H1), SEQ ID NO: 1738 (700344878H1), SEQ ID NO: 2014 (700345384H1), SEQ ID NO: 390 (700342666H1), SEQ ID NO: 401 (700342682H1), SEQ ID NO: 3512 (700380263H1), SEQ ID NO: 3441 (700380096H1), SEQ ID NO: 2370 (700346C08H1), SEQ ID NO: 1323 (700344124H1), SEQ ID NO: 634 (700343087H1), SEQ ID NO: 1986 (700345330H1), SEQ ID NO: 238 (700342403H1), SEQ ID NO: 987 (7003436C3H1), SEQ ID NO: 273 (700342463H1), SEQ ID NO: 208 (700342340H1), SEQ ID NO: 2674 (700346475H1), SEQ ID NO: 3119 (700347222H1), SEQ ID NO: 2203 (700345716H1), SEQ ID NO: 2186 (700345686H1), SEQ ID NO: 1307 (700344096H1), SEQ ID NO: 1372 (700344196H1), SEQ ID NO: 3053 (700347093H1), SEQ ID NO: 2507 (700346218H1), SEQ ID NO: 1049 (700343703H1), SEQ ID NO: 391 (700342667H1), SEQ ID NO: 3092 (700347176H1), SEQ ID NO: 3389 (700379975H1), SEQ ID NO: 326 (700342560H1), SEQ ID NO: 3605 (700380575H1), SEQ ID NO: 2946 (700346918H1), SEQ ID NO: 907 (700343487H1), SEQ ID NO: 3366 (700379925H1), SEQ ID NO: 1237 (700343991H1), SEQ ID NO: 3610 (700380584H1), SEQ ID NO: 2464 (700346146H1), SEQ ID NO: 3650 (700380683H1), SEQ ID NO: 2024 (700345405H1), SEQ ID NO: 1145 (700343841H1), SEQ ID NO: 1590 (700344655H1), SEQ ID NO: 2219 (700345747H1), SEQ ID NO: 224 (700342363H1), SEQ ID NO: 1108 (700343789H1), SEQ ID NO: 344 (700342589H1), SEQ ID NO: 2995 (700346996H1), SEQ ID NO: 3282 (700379719H1), SEQ ID NO: 1826 (700345020H1), SEQ ID NO: 1443 (700344334H1), SEQ ID NO: 3021 (700347040H1), SEQ ID NO: 558 (700342952H1), SEQ ID NO: 2292 (700345873H1), SEQ ID NO: 2030 (700345412H1), SEQ ID NO: 1005 (700343632H1), SEQ ID NO: 254 (700342432H1), SEQ ID NO: 1506 (700344454H1), SEQ ID NO: 1146 (700343842H1), SEQ ID NO: 1756 (700344911H1), SEQ ID NO: 2471 (700346157H1), SEQ ID NO: 1170 (700343875H1), SEQ ID NO: 1765 (700344928H1), SEQ ID NO: 1585 (700344649H1), SEQ ID NO: 495 (700342835H1), SEQ ID NO: 392 (700342670H1), SEQ ID NO: 5179 (700355947H1), SEQ ID NO: 4225 (700353526H1), SEQ ID NO: 5639 (700382063H1), SEQ ID NO: 3797 (700352605H1), SEQ ID NO: 4188 (700353271H1), SEQ ID NO: 5940 (700382691H1), SEQ ID NO: 4388 (700353955H1), SEQ ID NO: 5962 (700382739H1), SEQ ID NO: 6034 (700382890H1), SEQ ID NO: 4988 (700355520H1), SEQ ID NO: 5105 (700355788H1), SEQ ID NO: 5943 (700382702H1), SEQ ID NO: 4276 (700353625H1), SEQ ID NO: 5267 (700356140H1), SEQ ID NO: 3698 (700282414H2), SEQ ID NO: 4942 (700355431H1), SEQ ID NO: 4803 (700355029H1), SEQ ID NO: 5171 (700355925H1), SEQ ID NO: 5366 (700356466H1), SEQ ID NO: 4940 (700355428H1), SEQ ID NO: 5895 (700382605H1), SEQ ID NO: 4403 (700354129H1), SEQ ID NO: 4830 (700355088H1), SEQ ID NO: 5695 (700382183H1), SEQ ID NO: 5440 (700356708H1), SEQ ID NO: 5526 (700357108H1), SEQ ID NO: 5529 (700357122H1), SEQ ID NO: 5371 (700356474H1), SEQ ID NO: 4029 (700353005H1), SEQ ID NO: 5099 (700355779H1), SEQ ID NO: 4100 (700353128H1), SEQ ID NO: 3790 (700282575H2), SEQ ID NO: 3835 (700352675H1), SEQ ID NO: 3943 (700352843H1), SEQ ID NO: 3856 (700352709H1), SEQ ID NO: 3874 (700352735H1), SEQ ID NO: 4959 (700355462H1), SEQ ID NO: 5010 (700355558H1), SEQ ID NO: 4336 (700353828H1), SEQ ID NO: 5420 (700356627H1), SEQ ID NO: 5751 (700382275H1), SEQ ID NO: 5301 (700356219H1), SEQ ID NO: 6184 (700383158H1), SEQ ID NO: 5003 (700355547H1), SEQ ID NO: 4800 (700355026H1), SEQ ID NO: 4610 (700354647H1), SEQ ID NO: 4789 (700355009H1), SEQ ID NO: 4466 (700354337H1), SEQ ID NO: 5278 (700356158H1), SEQ ID NO: 4742 (700354908H1), SEQ ID NO: 5260 (700356129H1), SEQ ID NO: 5661 (700382106H1), SEQ ID NO: 3806 (700352616H1), SEQ ID NO: 4555 (700354518H1), SEQ ID NO: 5232 (700356066H1), SEQ ID NO: 5216 (700356023H1), SEQ ID NO: 6002 (700382827H1), SEQ ID NO: 4217 (700353446H1), SEQ ID NO: 4793 (700355016H1), SEQ ID NO: 4204 (700353409H1), SEQ ID NO: 6178 (700383149H1), SEQ ID NO: 6083 (700382979H1), SEQ ID NO: 4876 (700355249H1), SEQ ID NO: 4031 (700353007H1), SEQ ID NO: 6098 (700383013H1), SEQ ID NO: 6102 (700383021H1), SEQ ID NO: 4421 (700354239H1), SEQ ID NO: 5946 (700382708H1), SEQ ID NO: 5214 (700356020H1), SEQ ID NO: 5703 (700382195H1), SEQ ID NO: 3750 (700282503H2), SEQ ID NO: 4212 (700353432H1), SEQ ID NO: 6090 (700382995H1), SEQ ID NO: 4713 (700354850H1), SEQ ID NO: 4197 (700353284H1), SEQ ID NO: 5692 (700382179H1), SEQ ID NO: 4727 (700354875H1), SEQ ID NO: 5924 (700382668H1), SEQ ID NO: 4052 (700353052H1), SEQ ID NO: 4111 (700353142H1), SEQ ID NO: 4043 (700353028H1), SEQ ID NO: 5723 (700382229H1), SEQ ID NO: 5560 (700357402H1), SEQ ID NO: 5603 (700357489H1), SEQ ID NO: 5700 (700382191H1), SEQ ID NO: 5875 (700382543H1), SEQ ID NO: 3811 (700352625H1), SEQ ID NO: 6081 (700382975H1), SEQ ID NO: 5186 (700355963H1), SEQ ID NO: 5813 (700382392H1), SEQ ID NO: 5155 (700355889H1), SEQ ID NO: 4632 (700354689H1), SEQ ID NO: 5674 (700382139H1), SEQ ID NO: 4279 (700353629H1), SEQ ID NO: 4625 (700354676H1), SEQ ID NO: 5586 (700357445H1), SEQ ID NO: 4054 (700353057H1), SEQ ID NO: 5724 (700382230H1), SEQ ID NO: 5699 (700382190H1), SEQ ID NO: 6181 (700383154H1), SEQ ID NO: 3982 (700352912H1), SEQ ID NO: 4654 (700354742H1), SEQ ID NO: 4737 (700354895H1), SEQ ID NO: 4321 (700353779H1), SEQ ID NO: 4934 (700355415H1), SEQ ID NO: 5543 (700357160H1), SEQ ID NO: 4933 (700355414H1), SEQ ID NO: 3713 (700282435H1), SEQ ID NO: 3787 (700282569H2), SEQ ID NO: 3789 (700282571H2), SEQ ID NO: 4814 (700355053H1), SEQ ID NO: 5555 (700357189H1), SEQ ID NO: 3876 (700352737H1), SEQ ID NO: 4208 (700353424H1), SEQ ID NO: 3827 (700352654H1), SEQ ID NO: 3863 (700352719H1), SEQ ID NO: 6130 (700383062H1), SEQ ID NO: 4041 (700353023H1), SEQ ID NO: 5101 (700355781H1), SEQ ID NO: 5792 (700382353H1), SEQ ID NO: 4983 (700355513H1), SEQ ID NO: 4089 (700353114H1), SEQ ID NO: 4348 (700353853H1), SEQ ID NO: 6182 (700383155H1), SEQ ID NO: 5716 (700382218H1), SEQ ID NO: 5102 (700355783H1), SEQ ID NO: 5859 (700382507H1), SEQ ID NO: 5442 (700356725H1), SEQ ID NO: 5998 (700382817H1), SEQ ID NO: 4037 (700353014H1), SEQ ID NO: 4404 (700354142H1), SEQ ID NO: 5734 (700382245H1), SEQ ID NO: 5392 (700356534H1), SEQ ID NO: 5675 (700382140H1), SEQ ID NO: 5746 (700382267H1), SEQ ID NO: 4102 (700353130H1), SEQ ID NO: 4536 (700354478H1), SEQ ID NO: 4219 (700353509H1), SEQ ID NO: 5151 (700355885H1), SEQ ID NO: 4268 (700353612H1), SEQ ID NO: 3774 (700282549H2), SEQ ID NO: 3772 (700282547H2), SEQ ID NO: 5283 (700356171H1), SEQ ID NO: 5647 (700382075H1), SEQ ID NO: 5778 (700382330H1), SEQ ID NO: 5014 (700355577H1), SEQ ID NO: 5825 (700382426H1), SEQ ID NO: 5866 (700382525H1), SEQ ID NO: 4556 (700354519H1), SEQ ID NO: 3821 (700352645H1), SEQ ID NO: 4762 (700354942H1), SEQ ID NO: 4981 (700355507H1), SEQ ID NO: 5540 (700357155H1), SEQ ID NO: 5476 (700356835H1), SEQ ID NO: 5650 (700382078H1), SEQ ID NO: 5750 (700382273H1), SEQ ID NO: 5100 (700355780H1), SEQ ID NO: 4553 (700354516H1), SEQ ID NO: 4923 (700355345H1), SEQ ID NO: 4922 (700355344H1), SEQ ID NO: 3773 (700282548H2), SEQ ID NO: 3715 (700282438H2), SEQ ID NO: 5022 (700355594H1), SEQ ID NO: 5516 (700356974H1), SEQ ID NO: 4451 (700354305H1), SEQ ID NO: 4938 (700355422H1), SEQ ID NO: 5436 (700356695H1), SEQ ID NO: 5742 (700382260H1), SEQ ID NO: 5242 (700356081H1), SEQ ID NO: 4766 (700354954H1), SEQ ID NO: 3792 (700282584H2), SEQ ID NO: 3795 (700282592H2), SEQ ID NO: 5569 (700357415H1), SEQ ID NO: 5142 (700355871H1), SEQ ID NO: 4305 (700353713H1), SEQ ID NO: 4559 (700354534H1), SEQ ID NO: 5188 (700355966H1), SEQ ID NO: 5088 (700355766H1), SEQ ID NO: 5286 (700356177H1), SEQ ID NO: 5294 (700356193H1), SEQ ID NO: 3995 (700352935H1), SEQ ID NO: 4615 (700354658H1), SEQ ID NO: 5453 (700356767H1), SEQ ID NO: 4667 (700354769H1), SEQ ID NO: 5832 (700382439H1), SEQ ID NO: 5839 (700382450H1), SEQ ID NO: 6101 (700383019H1), SEQ ID NO: 6097 (700383011H1), SEQ ID NO: 4951 (700355447H1), SEQ ID NO: 4423 (700354247H1), SEQ ID NO: 4672 (700354777H1), SEQ ID NO: 4682 (700354793H1), SEQ ID NO: 5395 (700356541H1), SEQ ID NO: 5311 (700356241H1), SEQ ID NO: 4522 (700354446H1), SEQ ID NO: 4460 (700354326H1), SEQ ID NO: 5493 (700356901H1), SEQ ID NO: 5507 (700356933H1), SEQ ID NO: 4512 (700354425H1), SEQ ID NO: 5217 (700356025H1), SEQ ID NO: 4159 (700353215H1), SEQ ID NO: 5480 (700356847H1), SEQ ID NO: 6123 (700383051H1), SEQ ID NO: 4452 (700354310H1), SEQ ID NO: 4499 (700354409H1), SEQ ID NO: 4808 (700355040H1), SEQ ID NO: 5800 (700382365H1), SEQ ID NO: 5710 (700382210H1), SEQ ID NO: 6030 (700382882H1), SEQ ID NO: 5461 (700356796H1), SEQ ID NO: 5548 (700357173H1), SEQ ID NO: 4790 (700355011H1), SEQ ID NO: 5201 (700355989H1), SEQ ID NO: 4286 (700353653H1), SEQ ID NO: 6103 (700383023H1), SEQ ID NO: 6099 (700383015H1), SEQ ID NO: 5317 (700356252H1), SEQ ID NO: 5399 (700356551H1), SEQ ID NO: 5667 (700382123H1), SEQ ID NO: 6176 (700383147H1), SEQ ID NO: 5084 (700355758H1), SEQ ID NO: 4566 (700354558H1), SEQ ID NO: 4845 (700355173H1), SEQ ID ND: 4819 (700355067H1), SEQ ID NO: 3775 (700282550H2), SEQ ID NO: 5273 (700356152H1), SEQ ID NO: 6111 (700383037H1), SEQ ID NO: 3984 (700352916H1), SEQ ID NO: 5481 (700356848H1), SEQ ID NO: 5309 (700356236H1), SEQ ID NO: 4456 (700354317H1), SEQ ID NO: 4824 (700355077H1), SEQ ID NO: 5240 (700356076H1), SEQ ID NO: 5121 (700355829H1), SEQ ID NO: 5418 (700356624H1), SEQ ID NO: 4603 (700354637H1), SEQ ID NO: 5718 (700382220H1), SEQ ID NO: 5747 (700382268H1), SEQ ID NO: 6189 (700383166H1), SEQ ID NO: 6129 (700383061H1), SEQ ID NO: 5494 (700356903H1), SEQ ID NO: 5508 (700356935H1), SEQ ID NO: 4797 (700355022H1), SEQ ID NO: 5691 (700382176H1), SEQ ID NO: 5862 (700382513H1), SEQ ID NO: 5917 (700382654H1), SEQ ID NO: 5328 (700356273H1), SEQ ID NO: 3971 (700352891H1), SEQ ID NO: 5463 (700356802H1), SEQ ID NO: 4588 (700354606H1), SEQ ID NO: 4342 (700353839H1), SEQ ID NO: 5376 (700356483H1), SEQ ID NO: 5136 (700355856H1), SEQ ID NO: 5154 (700355888H1), SEQ ID NO: 5916 (700382649H1), SEQ ID NO: 5869 (700382537H1), SEQ ID NO: 5231 (700356062H1), SEQ ID NO: 4194 (700353279H1), SEQ ID NO: 4978 (700355496H1), SEQ ID NO: 5923 (700382665H1), SEQ ID NO: 4755 (7003549301), SEQ ID NO: 4008 (700352961H1), SEQ ID NO: 5424 (700356644H1), SEQ ID NO: 4035 (700353012H1), SEQ ID NO: 5323 (700356265H1), SEQ ID NO: 4818 (700355065H1), SEQ ID NO: 5367 (700356468H1), SEQ ID NO: 5888 (700382579H1), SEQ ID NO: 3690 (700282401H2), SEQ ID NO: 3794 (700282591H2), SEQ ID NO: 3764 (700282522H2), SEQ ID NO: 3758 (700282514H2), SEQ ID NO: 5128 (700355842H1), SEQ ID NO: 3818 (700352636H1), SEQ ID NO: 3781 (700282561H2), SEQ ID NO: 3782 (700282562H2), SEQ ID NO: 4889 (700355286H1), SEQ ID NO: 6104 (700383026H1), SEQ ID NO: 5138 (700355861H1), SEQ ID NO: 5140 (700355866H1), SEQ ID NO: 6006 (700382834H1), SEQ ID NO: 5880 (700382554H1), SEQ ID NO: 6116 (700383043H1), SEQ ID NO: 5774 (700382324H1), SEQ ID NO: 4024 (700352993H1, SEQ ID NO: 4820 (700355070H1), SEQ ID NO: 5438 (700356702H1), SEQ ID NO: 4150 (700353201H1), SEQ ID NO: 5909 (700382636H1), SEQ ID NO: 5551 (700357183H1), SEQ ID NO: 5055 (700355708H1), SEQ ID NO: 4551 (700354508H1), SEQ ID NO: 3975 (700352903H1), SEQ ID NO: 5793 (700382354H1), SEQ ID NO: 5823 (700382424H1), SEQ ID NO: 3915 (700352802H1), SEQ ID NO: 5146 (700355877H1), SEQ ID NO: 5879 (700382550H1), SEQ ID NO: 5627 (700382041H1), SEQ ID NO: 4211 (700353431H1), SEQ ID NO: 5894 (700382604H1), SEQ ID NO: 5806 (700382377H1), SEQ ID NO: 5558 (700357194H1), SEQ ID NO: 5396 (700356542H1), SEQ ID NO: 3892 (700352760H1), SEQ ID NO: 5211 (700356015H1), SEQ ID NO: 4011 (700352967H1), SEQ ID NO: 5282 (700356167H1), SEQ ID NO: 5978 (700382773H1), SEQ ID NO: 4898 (700355307H1), SEQ ID NO: 5522 (700356984H1), SEQ ID NO: 5226 (700356047H1), SEQ ID NO: 3777 (700282554H2), SEQ ID NO: 3776 (700282553H2), SEQ ID NO: 5817 (700382401H1), SEQ ID NO: 5858 (700382494H1), SEQ ID NO: 4627 (700354678H1), SEQ ID NO: 5411 (700356591H1), SEQ ID NO: 4257 (70035359051), SEQ ID NO: 4833 (700355093H1), SEQ ID NO: 4148 (700353195H1), SEQ ID NO: 5210 (700356014H1), SEQ ID NO: 4422 (700354244H1), SEQ ID NO: 4949 (700355444H1), SEQ ID NO: 4481 (700354371H1), SEQ ID NO: 6095 (700383009H1), SEQ ID NO: 4670 (700354774H1), SEQ ID NO: 4680 (700354790H1).

Analogous to the nucleic acid sequences, a CDP may have an amino acid sequence which is naturally occurring, synthetic, or variant. Guidance in determining which amino acid residues may be substituted, inserted, or deleted without abolishing biological or immunological activity may be found using computer programs such as LASERGENE Navigator (DNASTAR, Madison Wis.) which are well known in the art.

Derivation of Nucleic Acid Sequences

In the present embodiment, mRNA was isolated from corn tassel and used to construct the SATMON021 and SATMON024 cDNA libraries. Random CDNA isolates were sequenced in part and analyzed using the homology programs described below. The sequences of the isolates are disclosed in the Sequence Listing. These cdps may contain either a partial or a full length open reading frame, or they may contain all or part of a regulatory element for a particular gene. This variation is attributed to the fact that many genes are several hundred, and sometimes several thousand, bases in length. With current technology, large genes cannot be cloned in their entirety because of vector limitations, incomplete reverse transcription of the first strand, or incomplete replication of the second strand. This is particularly common in libraries generated by random priming, since first strand synthesis may begin anywhere within the transcript. Contiguous, secondary clones containing additional nucleotide sequences may be obtained using a variety of methods known to those of skill in the art.

Sequencing of the cDNAs

Methods for DNA sequencing are well known in the art. Conventional enzymatic methods employ DNA polymerase, Klenow fragment, THERMO SEQUENASE DNA polymerase (Amersham Pharmacia Biotech, Piscataway N.J.), or Taq DNA polymerase (Amersham Pharmacia Biotech) to extend the nucleic acid sequence from an oligonucleotide primer annealed to the DNA template of interest. Methods have been developed for the use of both single-stranded and double-stranded templates. Chain termination reaction products may be electrophoresed on urea-polyacrylamide gels and detected either by autoradiography (for radionucleotide-labeled nucleotides) or by fluorescence (for fluorescent-labeled nucleotides). Recent improvements in mechanized reaction preparation, sequencing, and analysis using the fluorescent detection method have permitted expansion in the number of inserts that may be sequenced per day using machines such as the ABI 377 DNA Sequencer (Perkin Elmer, Norwalk Conn.).

Reading Frame Determinations

The reading frame of the nucleotide sequence may be ascertained by several types of analyses. First, reading frames contained within the coding sequence may be analyzed for the presence of start (ATG, GTG, etc.) and stop codons (TGA, TAA, TAG). Typically, one reading frame will continue throughout the major portion of a cDNA sequence while the other two reading frames tend to contain numerous stop codons. For more difficult cases, algorithms have been created to analyze the occurrence of individual nucleotide bases at each putative codon triplet (Fickett, J. W. (1982) Nucl. Acids Res. 10:5303-5318). Coding sequences for particular organisms (bacteria, plants, and animals) tend to contain certain triplet periodicities, such as a significant preference for pyrimidines in the third codon position. These preferences have been incorporated into widely available software which may be used to determine the coding potential and frame of a given stretch of DNA. Coding preferences and start/stop codon information may be used to determine proper frame with a high degree of certainty which, in turn, permits cloning of the sequence in the correct frame.

The nucleotide sequences of the Sequence Listing have been prepared by current, state-of-the-art, automated methods and, as such, may contain occasional sequencing errors and unidentified nucleotides. Such unidentified nucleotides are designated by an N. The infrequent sequencing errors or N's in the nucleotide sequences of the Sequence Listing do not present a problem to those skilled in the art who wish to practice the invention. Several methods employing standard recombinant techniques, described in Ausubel, F. M. et al. (1997; Short Protocols in Molecular Biology, John Wiley & Sons, New York N.Y.), Sambrook, J. et al. (1989; Molecular Cloning, A Laboratory Manual, Cold Spring Harbor Press, Plainview N.Y.), or periodic updates thereof, may be used to correct errors and complete the missing sequence information. The same techniques used for obtaining a full-length sequence may be used to obtain a complete and accurate nucleotide sequence.

Homology Searches

The nucleic acid sequences of the Sequence Listing were used as query sequences against GenBank, or other databases available to the public, to determine homology to known sequences. Illustrative of computer programs known to those of skill in the art for performing computer-assisted nucleic acid or amino acid homology searches is the program Basic Local Alignment Search Tool or BLAST (Altschul, S. F. (1993) J. Mol. Evol. 36:290-300; Altschul, et al. (1990) J. Mol. Biol. 215:403-410). BLAST produces alignments of both nucleotide and amino acid sequences to determine sequence similarity. BLAST is especially useful in determining exact matches or homology. GenBank databases may be searched for sequences containing regions of homology to a query cdp of the present invention. Other databases (such as SwissProt, BLOCKS, or Pima II) may be searched for regions of amino acid sequence homology corresponding to the deduced CDP.

As described in Karlin (supra), the fundamental unit of BLAST algorithm output is the High-scoring Segment Pair (HSP). An HSP consists of two sequence fragments of arbitrary, but equal lengths, whose alignment is locally maximal and for which the alignment score meets or exceeds a threshold or cutoff score set by the user. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the BLAST program output.

BLAST may be used with any of the cdps of the present invention to search for HSPs between a query sequence and sequences in a reference nucleotide or protein database. The statistical significance of any matches is evaluated, and those matches that satisfy the user-selected threshold of significance are reported.

Homologous sequences, as determined by a BLAST search, may include prokaryotic (bacterial) or eukaryotic (animal, fungal, or plant) sequences. Where a cdp represents only part of a cene, the degree of homology is based only on the partial sequence disclosed in the Sequence Listing. When sequences have sufficiently long regiors of agreement or sufficiently high overall agreement, the score of the cdp is considered to be a nearly exact match. Allelic sequences fit this category when they only differ by about three nucleotides per 100. Homologous matches between the cdps provided in the Sequence Listing and the GenBank databases are reported in TABLES 1 and 2.

Corn Tassel-Derived Sequences

The cdps of the present inventior may serve to identify, evaluate, alter, or follow the inheritance of desired characteristics associated with growth and development, disease resistance, environmental adaptability, quality, and yield of corn. In particular, cdps are useful as molecular markers for studying inheritance of multigene traits in a plant breeding program.

Hybridization and Genetic Analysis

The cdps, their oligonucleotides, fragments, or complementary sequences, may be used to identify the presence of and/or to determine the degree of similarity between two (or more) nucleic acid sequences. The cdps may be hybridized to naturally occurring or recombinant nucleic acid sequences under appropriately selected temperatures anct salt concentrations. Hybridization with a probe based on the nucleic acid sequence of at least one of the cdps allows for the detection of nucleic acid sequences, including genomic sequences, which are identical or closely homologous to the cdps of the Sequence Listing. Probes may be selected from non-conserved or unique regions of the cdps of interest and pretested for their ability to identify or amplify the target nucleic acid sequence using standard protocols. Optimization of the protocol, e.g. increasing stringency to reduce the frequency of false positives or avoiding polyadenylated or other regions predicted to provide secondary structure to reduce false negatives, should provide the desired results. A labeled probe may be used to detect or quantify cDNAs, endogenous corn transcripts, or genes. As will be understood by those of skill in the art, hybridization conditions, probe length and labeling will vary depending upon the intended use. Hybridization conditions, based on the melting temperature (Tm) of the probe and on the salt concentrations under which hybridization and subsequent washes are carried out are well known in the art and are taught in Sambrook (supra) and Ausubel (supra).

A probe for use in Southern or northern hybridization may be a cdp sequence or its complement that is up to several hundred nucleotides long and either single-stranded or double-stranded. Such probes may be hybridized in solution to biological materials such as plasmids, bacterial or yeast artificial chromosomes, cleared plant tissues, etc. or to artificial substrates containing cdps. Microarrays are particularly suitable for identifying the presence and detecting the level of gene expression of multiple desired traits by examining gene expression of selected inbreds and hybrids at various stages of development. An array analogous to a dot or slot blot may be used to arrange and link the cDNA fragments or oligonucleotides to the surface of a substrate using one or more of the following: mechanical (vacuum), chemical, thermal, or UV bonding procedures. Such an array may contain any number of cdps and may be produced by hand or by using available devices, materials, and machines.

Probes may be labeled by either PCR or enzymatic techniques using a variety of reporter molecules. Commercial kits are available for radioactive labeling and probe cleanup from Amersham Pharmacia Biotech, for alkaline phosphatase labeling from Life Technologies (Rockville Md.), for chemiluminescent labeling from Lumigen (Southfield Mich.), etc. Alternatively, cdps may be cloned into commercially available vectors for the production of RNA probes. Such probes may be transcribed in the presence of at least one labeled nucleotide (e.g. [α-³²P] CTP, Amersham Pharmacia Biotech).

Genetic maps, based upon molecular markers (restriction fragment length polymorphisms, RFLPs) are being assembled for several grains including rice, corn, barley, and wheat. These maps have improved understanding and manipulation of both single and multigene traits. Even when the genes involved are unknown, the ability to show the presence of the associated marker and the desired characteristics in inbred or hybrid corn plants and to follow segregation in a breeding program make the marker valuable as a diagnostic. Moreover, continuous variation within a segregating family may often be resolved into a handful of major gene effects associated with molecular markers. As genetic maps merge with physical maps, it becomes possible to walk along the chromosome and clone virtually any gene. Hybridization and newer technologies such as random amplified polymorphic DNA (RAPD) analysis, microsatellites and amplified fragment length polymorphisms (AFLP) make it easier to isolate the actual genes which interact and are responsible for a desired trait.

Diagnostic Uses

Diagnostic assays known to those of skill in the art may be used to detect or confirm conditions or diseases associated with abnormal levels of cdp expression. Labeled probes developed from the nucleotide sequence encoding a cdp are added to a plant sample under amplifying or hybridizing conditions. The complex between the naturally occurring sequence and the labeled probe is quantified and compared with a standard for that cell or tissue. If cdp expression varies significantly from the standard, the assay indicates the presence of the condition or disease. Qualitative or quantitative diagnostic methods may include northern, dot blot, or other membrane or dip-stick based technologies or multiple-sample format technologies such as PCR, ELISA-like, pin, or chip-based assays. The determination of whether cdp expression in a sample varies significantly from a standard is determined by methods of statistical analyses well known to those of skill in the art.

Accordingly, the invention provides a method for assessing disease resistance or other conditions using a panel of probes. A candidate probe is identified from CDPs which are specific to corn tissue and have not been observed in GenBank or other Incyte-sequenced cDNA libraries. The usefulness of the probe may be tested by quantifying its hybridization across tissues which are normal versus diseased. Once an increase (or decrease) in expression level is related to a trait such as fungal resistance, the probe can be used to monitor ability of a particular inbred or hybrid corn line to withstand fungal infection.

Transcriot Imaging

Another embodiment relates to development of diagnostic or treatment methods based on specific imaging of the cdps of the present invention. The profile of nucleic acid sequences which reflect gene transcription activity in a particular cell type, tissue, or plant at a particular time, is defined as a “transcript image”. Such profiles are generated by naming, matching, and counting all copies of related clones and arranging them in order of abundance.

Clones may also be arranged in clusters in descending order of abundance. The minimum number of clones necessary to constitute a cluster, as illustrated at the bottom of TABLE 2, is two. All clones in TABLE 2 are tassel specific although individual clusters may consist of either unique cdps or cdps that are homologous to known sequences. An alternative presentation of this data might involve a spreadsheet which contains cluster abundance data as well as some descriptive information for the homologous clones.

Subtractions, or subsetting, among transcript images may be used to discern various differences in gene expression and cellular activities. For example, subsetting may be used with the PHYTOSEQ database (Incyte Pharmaceuticals, Palo Alto Calif.) to show differences between: a) organs of two different developmental stages; b) two different organs, such as leaves and roots; c) organs from two different species; or d) normal and diseased or stressed plant tissues.

Large numbers of mRNA transcripts, as represented by their respective cDNA clones, may be compared using computational methods rather than analogous laboratory methods, such as northern blot analysis. For example, electronic subtraction between any two transcript images parallels hybrid subtraction between any two cDNA libraries (cf. Sambrook, supra) . The information produced by the subtraction of transcript images between different libraries may be used to select single or multiple cdps which may be used to predict yield.

A cdp identified through transcript imaging, or other means, may also be used to clone regulatory elements for use in transformation vectors. Expression may be quantified using amplification or microarray technologies which are well known in the art.

Complementary Strand

The cdp, or any part thereof, may be used as a tool in technologies for altering gene expression. To inhibit in vivo or in vitro cdp transcription, a PNA (Nielsen, P. E. et al. (1993) Anticancer Drug Des. 8:53-63) or an oligonucleotide based on the sequence of a cdp is designed using OLIGO 4.06 software (National Biosciences, Plymouth Minn.) or LASERGENE Navigator (DNASTAR). Alternatively, a fragment of a cdp is cloned into an expression vector which is transformed into a host cell to express the complementary strand. An analogous molecule may be designed to inhibit promoter binding in the upstream nontranslated leader or at various sites along the 5′ coding region of the cdp. Alternatively, complementary molecules may be designed to inhibit translation of an mRNA by preparing an oligomer or fragment which binds to the transcript preventing its association with the ribosomal machinery.

Complementary molecules may also be designed to disrupt genomic sequences (such as enhancers, introns) preventing the normal activity of these regulatory elements. Similarly, complementary strands may be used in a process known as “triple helix” base pairing to inhibit replication. These molecules compromise the ability of the double helix to open and bind to polymerases and transcription factors necessary for replication.

Stable transformation of appropriate dividing cells with an expression vector encoding the complement of a cdp may produce a transgenic cell line, tissue, or organism. Those cells which assimilate, replicate, and express the nucleic acid sequence in sufficient quantities may compromise or entirely eliminate the natural activity of the cdp. Frequently, the function of a cdp may be ascertained by observing lethality, loss of physiological activity, changes in morphology, etc. at the cellular, tissue, organ, or organismal level.

Expression

The cdps may be used in recombinant vectors to express a polypeptide. It may be advantageous to design nucleic acid sequences possessing the GC ratio of codons preferred by a particular prokaryotic or eukaryotic host (Murray, E. et al. (1989) Nuc. Acids Res. 17:477-508). In addition, 3′ terminators, such as bacterial nopalene synthase or octapine synthase, may be modified, or substituted into vectors, to produce transcripts having more desirable properties, such as a longer half-life, than transcripts produced from the naturally occurring sequence (Sullivan, M. L. and Green, P. J. (1993) Plant Mol. Biol. 23:1091-1104; Silva, E. M. et al. (1987) J. Cell Biol. 105:245). The cdps may also be altered by site-directed mutagenesis to insert new restriction sites and to modify the peptide by glycosylation, phosphorylation, acetylation, etc.

The cdp may be ligated to a heterologous sequence to create a chimeric or fusion protein. For ease of purification, it may be useful to produce a fusion protein that is recognized by a commercially available antibody. In addition, the sequence may be engineered to introduce a cleavage site between the peptide of interest and the heterologous protein sequence, so that the peptide may be cleaved from the heterologous moiety and purified.

Alternately, the peptide may be synthesized, whole or in part, using chemical methods well known in the art. For example, peptides may be synthesized using various solid-phase techniques (Roberge, J. Y. et al. (1995) Science 269:202-204) or an Peptide Synthesizer Model 431A (Perkin Elmer) using instructions provided by the manufacturer. Once synthesized, the peptide may be purified by preparative high performance liquid chromatography, and composition confirmed by amino acid sequencing (Ausubel (supra) p. 10.82f).

Expression Systems

For protein expression, the nucleic acid sequence may be inserted into an expression vector which contains the necessary elements for appropriate transcription and translation. Methods which are well known to those skilled in the art may be used to construct such vectors. These methods include in vitro recombinant DNA techniques, synthetic techniques, in vivo recombination, or genetic recombination. Such techniques are described in Sambrook (supra) and Ausubel (supra). One of the advantages of producing the CDPs by recombinant DNA technology is the ability to obtain highly-enriched sources of the polypeptides that simplify purification procedures.

The cdps may be engineered into a variety of expression vectors and host cells. These include, but are not limited to, microorganisms such as bacteria transformed with recombinant bacteriophage, plasmid or cosmid DNA expression vectors; yeast transformed with yeast expression vectors; insect cell systems infected with virus expression vectors (e.g. baculovirus); plant cells transfected with expression vectors containing viral, bacterial, or eukaryotic elements (e.g. cauliflower mosaic virus, CaMV; Ti or pBR322 plasmids; and cell or tassel-specific, constitutive or inducible, monocot or corn elements).

The regulatory elements of vectors vary in their strength and specificities and are those nontranslated regions such as enhancers, promoters, introns, and 3′ untranslated regions which interact with host proteins to carry out transcription and translation. Depending on the vector and host, any number of suitable transcription and translation elements may be used. For example, promoters or enhancers derived from the genomes of plant cells (e.g. heat shock, RUBISCO; and storage protein genes) or from plant viruses (e.g. viral promoters or leader sequences) may be cloned into the vector containing an appropriate selectable marker. In fact, the cdps of this invention may be used to clone upstream, tissue-specific or inducible regulatory elements for purposes of engineering and expressing heterologous genes in corn.

In a bacterial system, an expression vector may be selected to direct a high level expression of a fusion protein. Commercial vectors include, but are not limited to, the multifunctional E. coli cloning and expression vectors, PBLUESCRIPT (Stratagene, La Jolla Calif.) and PSPORT (Life Technologies). Using either of these vectors, the nucleic acid sequence may be ligated into the vector in-frame with sequences for the amino-terminal Met and the subsequent 7 residues of β-galactosidase so that a chimeric protein is produced. PGEX vectors (Amersham Pharmacia Biotech) may also be used to express peptides by ligating the nucleic acid sequence to glutathione S-transferase (GST). In general, such fusion proteins are soluble and may easily be purified from lysed cells by adsorption to glutathione-agarose beads followed by elution in the presence of free glutathione. Proteins made in such systems are designed to include heparin, thrombin, or factor X_{A} protease cleavage sites so that the peptide of interest may be released from the GST moiety at will.

In plants, the expression of nucleic acid sequences may be driven by any of a number of promoters. Viral promoters such as the 35S and 19S promoters of CaMV may be used alone or in combination with the omega leader sequence from TMV (Takamatsu, N. et al. (1987) EMBO J. 6:307-311). Alternatively, plant promoters such as the small subunit of RUBISCO (Coruzzi, G. et al. (1984) EMBO J. 3:1671-1680; Broglie, R. et al. (1984) Science 224:838-843); or heat shock promoters (Winter, J. and Sinibaldi, R. M. (1991) Results Probl. Cell Differ. 17:85-105) may be used. Preferably, the cdps of the invention may be used to identify clones containing full length genes by hybridization or to clone full length genes or regulatory elements for use in expression vectors by PCR.

X-Ray Crystallography

Expression of the recombinant CDP in sufficient amounts may make analytical studies such as X-ray crystallography possible. In the alternative, knowledge of the amino acid sequence deduced from the nucleic acid sequence may provide guidance to those employing computer modeling techniques in place of or in addition to X-ray crystallography.

Antibodies

Anti-CDP antibodies may be produced to use in assays of protein expression. Such antibodies include, but are not limited to, polyclonal, monoclonal, chimeric, single chain, Fab fragments and fragments produced by a Fab expression library. Neutralizing antibodies, i.e., those which inhibit dimer formation, are especially useful for diagnostics.

The amino acid sequence encoded by the cdps of the Sequence Listing may be analyzed by appropriate software (e.g. LASERGENE Navigator, DNASTAR) to determine regions of high immunogenicity. The optimal sequences for immunization are selected from the C-terminus, the N-terminus, and those intervening, hydrophilic regions of the peptide which are likely to be exposed to the external environment when the peptide is in its natural conformation. Analysis used to select appropriate epitopes is also described by Ausubel (supra, unit 11-7). Peptides used for antibody induction do not need to have biological activity; however, they must be antigenic. Peptides used to induce specific antibodies may have an amino acid sequence consisting of at least five amino acids, and preferably at least 10 amino acids. An oligopeptide should mimic an antigenic portion of the natural peptide and may be fused with another protein such as KLH (Sigma-Aldrich) for antibody production. An oligopeptide or peptide encompassing an antigenic region may be expressed from the nucleic acid sequence, synthesized, or purified from corn.

Procedures well known in the art may be used for the production of antibodies. Various hosts including mice, goats, and rabbits, may be immunized by injection with a peptide or oligopeptide. Depending on the host species, various adjuvants may be used to increase immunological response.

In one procedure, oligopeptides about 15 residues in length may be synthesized using a Peptide Synthesizer Model 431A (Perkin Elmer) using Fmoc-chemistry and coupled to KLH (Sigma-Aldrich) by reaction with M-maleimidobenzoyl-N-hydroxysuccinimide ester (Ausubel, supra) . If necessary, a cysteine may be introduced at the N-terminus of the oligopeptide to permit coupling to KLH. Rabbits are immunized with the oligopeptide-KLH complex in complete Freund's adjuvant. The resulting antisera are tested for antipeptide activity by binding the peptide to plastic, blocking with 1% BSA, reacting with rabbit antisera, washing, and reacting with radioiodinated goat anti-rabbit IgG.

In another procedure, the peptide, in quantities up to 75 mg, may be used to immunize mice or rabbits. About 100 μg are used to immunize a mouse, while up to 1 mg is used to immunize a rabbit. Subsequently, the peptide is radioiodinated and used to screen the B-lymphocyte cells from the immunized animal for production of hybridomas using standard techniques. About 20 mg of protein are sufficient for labeling and screening several thousand clones.

Hybridomas may also be prepared and screened using standard techniques. Hybridomas of interest are detected by screening with radioiodinated peptide to identify those fusions producing peptide-specific monoclonal antibody. In a typical protocol, wells of microtiter plates are coated with affinity-purified, specific rabbit-anti-mouse (or suitable anti-species IgG) antibodies at 10 mg/ml. The coated wells are blocked with 1% BSA and washed and exposed to supernatants from hybridomas. After incubation, the wells are exposed to radiolabeled peptide at 1 mg/ml. Clones producing antibodies bind a quantity of labeled peptide that is detectable above background.

Such clones are expanded and subjected to 2 cycles of cloning at 1 cell/3 wells. Cloned hybridomas are injected into pristane-treated mice to produce ascites, and monoclonal antibody is purified from the ascitic fluid by affinity chromatography on Protein A (Amersham Pharmacia Biotech). Monoclonal antibodies with affinities of at least 10⁸ M⁻¹, preferably 10⁹ M⁻¹ to 10¹⁰ M⁻¹ or greater, are made by standard procedures as described in Harlow (1988; Antibodies: A Laboratory Manual, Cold Spring Harbor Laboratory, Cold Spring Harbor N.Y.) and Goding (1986; Monoclonal Antibodies: Principles and Practice, Academic Press, New York N.Y.).

Antibody fragments which contain specific binding sites for an epitope may also be generated. For example, such fragments include, but are not limited to, the F(ab′)2 fragments which may be produced by pepsin digestion of the antibody molecule and the Fab fragments which may be generated by reducing the disulfide bridges of the F(ab′)2 fragments. Alternatively, Fab expression libraries may be constructed to allow rapid and easy identification of monoclonal Fab fragments with the desired specificity (Huse, W. D. et al. (1989) Science 256:1275-1281).

Assays Using Antibodies

Anti-CDP antibodies may be used to determine the amount of CDP found in a particular cell of a corn inbred line or hybrid under various environmental or disease conditions. Assays for such peptides include methods utilizing the antibody and a label to detect expression in plant extracts, cells, or tissues. The peptides and antibodies of the invention may be used with or without modification. Frequently, the peptides and antibodies will be labeled by joining them, either covalently or noncovalently, with a reporter molecule.

Protocols for detecting and measuring protein expression using either polyclonal or monoclonal antibodies, are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes on a peptide is preferred, but a competitive binding assay may be employed. Such immunoassays typically involve the formation of complexes between the CDP and its specific antibody and the measurement of such complexes. These and other assays are described, among other places, in Hampton, R. et al. (1990, Serological Methods, a Laboratory Manual, APS Press, St Paul Minn.) and Maddox, D. E. et al. (1983, J. Exp. Med. 158:1211-1226).

Screening for Useful Compounds

The cdps or CDPs are particularly useful for screening libraries of molecules or test compounds for identification of those molecules which bind specifically to them. For example, a cdp or fragment thereof may be combined with a plurality of natural, synthetic, or inorganic molecules to screen for molecules that bind to them and function as transcription factors, enhancers, or other cellular elements which contribute to gene expression. Similarly, a CDP or portion thereof may be combined with a plurality of molecules to screen for molecules which bind to them. Molecules identified by screening with cdps or CDPs preferably affect growth and development, disease resistance, environmental adaptability, quality, or yield.

Technologies with multiple-sample format, ELISA-like, capillary, or chip-based assays, are well known in the art and allow large-scale screening. These methods use complex formation, quantification, and comparison with a standard to detect molecules which specifically bind the cdps or CDPs. In the assay, the cdp or CDP may be free in solution, affixed to a substrate, borne on a cell surface, or located intracellularly. For example, prokaryotic host cells which are stably transformed with recombinant nucleic acids that express and position a CDP on the cell surface can be used to screen for molecules which specifically bind the CDP. Viable or fixed cells are screened against a plurality of test compounds and the specificity of binding or formation of complexes between an expressed CDP and the test compound is measured.

Transformation

Bacterial and plant transformation systems are well known in the art. Expression vectors may be introduced into suitable E. coli cells by electroporation, heat shock or other means as described in Ausubel (supra) for the purpose of expressing a plant protein. Expression vectors may also be introduced into plant cells by direct transfer of DNA or pathogen-mediated transfection. For reviews, see McGraw Hill Yearbook of Science and Technology (1992; McGraw Hill New York N.Y., pp 191-196); or Weissbach, A. and Weissbach, H. (1988; Methods Enzymol. 118:421-463). Direct transfer of DNA into plant protoplasts or cells is one approach for transforming plants genetically. DNA uptake by protoplasts may be promoted chemically with polyethylene glycol or electrically with a high-voltage pulse. Both of these methods depend upon a cell culture system to recover plants from a single transformed cell (Rhodes, C. A. et al. (1988) Biotechnology 6:56-60; Morocz, S. (1991) Theor. Appl. Genet. 80:721-726). Regeneration of transformed, fertile plants has been demonstrated in several cereals including rice (Zhang, H. M. (1988) Plant Cell Rep. 7:379-384).

Electroporation, lipofection, microinjection, particle bombardment, vacuum infiltration, and electrotransformation may be used to transform corn cells and embryos. Gordon-Kamm, W. J. et al. (1992; Plant Mol. Biol. 18:201-210) used particle bombardment to transform embryogenic, suspension culture cells; Murry, L. E. et al. (In: Bajaj, Y. P. S. (1994) Biotechnology in Agriculture and Forestry 25:252-261) used continuous, low voltage electric current to transform embryos; and Rhodes, C. A. et al. (1995; Methods Mol. Biol. 55:121-131) describe the electroporation of embryos. Stable transformation requires the use of an expression vector which contains an appropriate origin of replication and gene cassettes containing viral or plant expression elements, a selectable or visible marker, and a gene of interest. Following the introduction of the vector, cells may be allowed to grow for 1-2 days in an enriched media. If the vector contains a selectable marker, the cells are switched to selective media. The selectable marker confers resistance to selective agents and allows growth and recovery of those cells which successfully express the introduced sequences.

Any number of selection systems may be used to recover transformed cell lines. Antimetabolite, antibiotic or herbicide resistance may be used as the basis for selection using genes such as dhfr, which confers resistance to methotrexate (Wigler, M. et al. (1980) Proc. Natl. Acad. Sci. 77:3567-3570); npt, which confers resistance to the aminoglycosides, neomycin and G-418 (Colbere-Garapin, F. et al. (1981) J. Mol. Biol. 150:1-14); and als or pat, which confer resistance to chlorsulfuron and phosphinotricin acetyltransferase, respectively (McGraw Hill Yearbook of Science and Technology, supra). Recently, the use of visible markers has gained popularity with such markers as anthocyanins, β glucuronidase and its substrate, GUS, luciferase and its substrate, luciferin, and green fluorescent protein, GFP, being widely used not only to identify transformants, but also to quantify the amount of transient or stable protein expression attributable to a specific vector system (Rhodes (1995) supra; Haseloff, J. and Amos, B. (1995) Trends Genet. 11:328-329). Plant expression vectors contain 5′ promoters, enhancers and 3′ terminators that will function in the plant cell.

Identification of Transformants

Although the presence/absence of marker gene expression suggests that the gene of interest is also present, its expression should be confirmed. For example, if the sequence is inserted within a marker gene sequence, cells containing the recombinant sequence may be identified by the absence of marker gene function. Alternatively, a marker gene may be placed in tandem with the nucleic acid sequence under the control of a single promoter. Expression of the marker gene in response to induction or selection may indicate the presence and expression of the tandem sequence as well.

Alternatively, host cells which contain the introduced nucleic acid sequence may be identified by a variety of procedures known to those of skill in the art. These procedures include, but are not limited to, DNA-DNA or DNA-RNA hybridization and protein bioassay or immunoassay techniques which include membrane, solution, gel, or chip based technologies for the detection and/or quantification of the nucleic or amino acids and any of the molecules to which they bind.

The presence of the nucleic acid sequence may be detected by DNA-DNA or DNA-RNA hybridization or amplification using probes comprising all or a portion of a nucleic acid sequence. Nucleic acid amplification based assays involve the use of oligonucleotides or oligomers based on the nucleic acid sequence to detect transformants containing the introduced DNA.

A wide variety of labels and conjugation techniques are known by those skilled in the art and may be used in various nucleic acid and amino acid assays. Means for producing labeled hybridization or PCR probes for detecting related sequences include oligolabeling, nick translation, end-labeling or PCR amplification and are well known in the art. Alternatively, the nucleic acid sequence may be cloned into a vector for the production of an mRNA probe. Such vectors are known in the art, are commercially available, and may be used to synthesize RNA probes in vitro by addition of an appropriate RNA polymerase such as T7, T3 or SP6 and labeled nucleotides. A number of companies (e.g., Amersham Pharmacia Biotech and Life Technologies) supply commercial kits, reporter molecules, and protocols for these procedures.

### EXAMPLES

### Isolation, Sequence Analysis and Use of Corn Sequences

I Growth Conditions

The corn cDNA libraries, SATMON021 and SATMON024, were constructed from corn tassel grown and prepared as follows. Seeds were planted 3 cm deep in soil in 2″-3″ peat pots containing Metro-Mix 200 growing medium (The Scotts Company, Marysville Ohio). After 2-3 weeks, the plants were transplanted into 10″ pots. After transplant, PETERS 15-16-17 fertilizer (The Scotts Company) at a strength of 150 ppm nitrogen was applied ˜3× per week. Iron (total ˜900 mg) was added to each pot two to three times between transplanting and flowering of the plant. Corn plants were grown in a greenhouse in 27° C./21° C., 15 hr day/9 hr night cycles. Lighting was provided by 1000 W sodium vapor lamps. For SATMON021, a corn plant was grown to the V8 stage at which time the tassel was 15 to 20 cm long. The tassel was harvested, frozen in liquid nitrogen, and stored at −80° C. For SATMON024, a corn plant was grown to the V9 stage. The rapidly developing tassel was harvested when 37 cm long with its concomitant glume, anthers and pollen. The tissue was frozen in liquid nitrogen and stored at −80° C.

II cDNA Library Construction

The mRNA was handled according to the recommended protocols in the SuperScript Plasmid System for cDNA synthesis and plasmid cloning (Life Technologies). The cDNAs were fractionated on a SEPHAROSE CL-4B column (Amersham Pharmacia Biotech). The cDNAs for SATMON021 and SATMON024 were ligated into the NotI and EcoRI sites of the pINCY plasmid (Incyte Pharmaceuticals). The plasmid pINCY was subsequently transformed into DH10B competent cells (Life Technologies).

III Isolation and Sequencing of cDNA Clones

The plasmid DNA was released from the cells and purified using the R.E.A.L. Prep 96 plasmid kit (Qiagen, Valencia Calif.). This kit enabled the simultaneous purification of 96 samples in a 96-well block using multi-channel reagent dispensers. The recommended protocol was employed except for the following changes: 1) the bacteria were cultured in 1 ml of sterile Terrific Broth (Life Technologies) with carbenicillin at 25 mg/l and glycerol at 0.4%; 2) after inoculation, the cultures were incubated for 19 hours and at the end of incubation, the cells were lysed with 0.3 ml of lysis buffer; and 3) following isopropanol precipitation, the plasmid DNA pellet was resuspended in 0.1 ml of distilled water. After the last step in the protocol, samples were transferred to a 96-well block for storage at 4° C.

In the alternative, DNA was isolated using the following protocols. Single bacterial colonies were transferred into individual wells of the 384-well plates (Genetix, Christchurch UK) using sterile toothpicks. The wells contained 1 ml of sterile Terrific Broth (Life Technologies) with 25 mg/l carbenicillin and 0.4% glycerol (v/v). The plates were covered and placed in a THERMODYNE incubator (Thermodyne Corp, Newtown Square Pa.) at 37° C. for 8-10 hours prior to use. Plasmid DNA was released from the cells and amplified using direct link PCR (Rao, V. B. (1994) Anal. Biochem. 216:1-14) as follows.

The direct link PCR solution included 30 ml of NUCLEIX PLUS PCR nucleotide mix (Amersham Pharmacia Biotech) and 300 μl of Taq DNA polymerase (Amersham Pharmacia Biotech) with or without 12 μl Pfu DNA polymerase (Stratagene). Five microliters of the PCR solution were added to each of the 384 wells using the Hydra-96 microdispenser (Hamilton, Reno Nev.); plates were centrifuged at 1000 rpm for 20 seconds and refrigerated until use. A 384 pin tool (V&P Scientific, San Diego Calif.) was used to transfer bacterial cells from the incubation plate into the plate containing the PCR solution where the component 0.1% Tween 20 (polyoxyethylene(20)sorbitan monolaurate) caused the cells to undergo lysis and release the plasmid DNA. After lysis, the plates were centrifuged up to 500 rpm, covered with a cycle sealer, and cycled using a 384-well Peltier Thermal Cycler (PCT-200; M J Research, Watertown Mass.) using the program dPCR30 with the following parameters: Step 1) 95° C., 1 minute; Step 2) 94° C., 30 seconds; Step 3) 55° C., 30 seconds; Step 4) 72° C., 2 minutes; Step 5) steps 2, 3, and 4 repeated 29 times; Step 6) 72° C., 10 minutes; and Step 7) storage at 4° C.

The concentration of DNA in each well was determined by dispensing 100 μl PICOGREEN quantitation reagent (Molecular Probes, Eugene Oreg.) (0.25% reagent dissolved in 10 mM TrisHCl, pH 7.5, 1 mM ethylenediamine tetraacetic acid (EDTA) (1× TE, v/v), and 0.5 μl of undiluted PCR product into each well of an opaque fluorimeter plate (Corning Costar, Acton Mass.) and allowing the DNA to bind to the reagent. The plate was scanned in a Fluoroskan II (Labsystems Oy, Helsinki, Finland) to measure the fluorescence of the sample and to quantify the concentration of DNA.

The cDNAs were prepared and sequenced by the method of Sanger, F. and A. R. Coulson (1975; J. Mol. Biol. 94:441-448), using either a MICROLAB 2200 system (Hamilton) or a HYDRA microdispenser (Robbins Scientific, Sunnyvale Calif.) in combination with PTC-200 cyclers (M J Research) and ABI PRISM 377 DNA sequencing systems (Perkin Elmer). Most of the isolates were sequenced according to standard PE protocols and kits (Cat. #79345, 79339, 79340, 79357, 79355). The solution volumes were used at 0.25×-1.0× concentrations. In the alternative, cDNAs may have been sequenced using solutions and dyes from Amersham Pharmacia Biotech.

IV Homology Searching of cDNA Clones and Their Deduced Proteins

After the reading frame was determined, the nucleotide sequences of the Sequence Listing or their deduced amino acid sequences were queried against databases such as GenBank, SwissProt, BLOCKS, and Pima II. These databases which contain annotated sequences were searched for regions of homology (similarity) using BLAST (Altschul (1993) supra; Altschul (1990) supra).

BLAST produced alignments of both nucleic and amino acid sequences to determine sequence similarity. Because of the local nature of the alignments, BLAST was especially useful in determining exact matches or in identifying homologs of viral, prokaryotic, or eukaryotic origin. Other algorithms such as the one described in Smith, T. F. (1992; Protein Engineering 5:35-51) could have been used to deal with primary sequence patterns and secondary structure gap penalties. The sequences disclosed in this application have lengths of at least 49 nucleotides, and no more than 12% uncalled bases (where N is recorded rather than A, C, G, or T).

SATMON021 and SATMON024 nucleotide sequences were searched, as described in Karlin (supra), against the GenBank plant (pln) and eukaryote (eukp) databases, and deduced amino acid sequences from the same clones were then searched against GenBank functional protein databases (allp). The relevant database for a particular match was reported in column 5 of TABLE 1. In TABLE 1 column 3, the product score is calculated as follows: the % nucleotide or amino acid identity [between the query and reference sequences] in BLAST is multiplied by the % maximum possible BLAST score [based on the lengths of query and reference sequences] and then divided by 100. In an analogy to the hybridization procedures used in the laboratory, the electronic stringency for an exact match was set at 70, and the conservative lower limit for an exact match was set at approximately 40 (with 1-2% error due to uncalled bases). Column 4 provides log-likelihood where the value=log (probability÷threshold) and the threshold for exact matches was set at 10-25 for nucleotides and 10⁻⁴ for peptides. Column 6 contains a GenBank description of the protein; some of the GenBank descriptions were standardized with respect to abbreviations and spelling.

V Gene Transcript Analysis

The abundance sort program of the invention described in U.S. Pat. No. 5,840,484 entitled “Comparative Gene Transcript Analysis”, incorporated herein by reference, tabulates and sorts by frequency the mRNA transcripts corresponding to each gene identified in a database. The process for obtaining this data set, the profile of corn tassel gene activity or transcript image, is referred to as “gene transcript analysis”.

A transcript analysis summarizes the presence and abundance of exact, unique, and homologous transcripts which are tassel specific. A transcript image may be assembled using TABLE 1, TABLE 2, and the Sequence Listing. Such a collection of sequences is used to characterize minimally active, active, or highly active cdps. Comparisons among normal, diseased, or immature tassel are used to identify those sequences of particular use in predicting yield or in recovering regulatory elements to be used in vectors for genetic engineering. The entire set, or a selected subset, of tassel-specific, unique, or homologous cDNAs may be useful in membrane-based or PCR-based diagnostic technologies.

VI Library Comparisons, Subsetting

LIFESEQ database (Incyte Pharmaceuticals) software is used to compare sets of transcript images for PHYTOSEQ database (Incyte Pharmaceuticals) cDNA libraries. The cdps are filtered by selecting desired values for relative abundance, stringency, and/or product score (described infra). For any particular library, only the subset of cdps that meet the selected values is included in the comparison. Additional filters, such as those to exclude common genes, such as ribosomal proteins, elongation factor, etc. may be used in the search operation. The subsetting of thousands of corn sequences from the transcript images of callus, ear, embryo, endosperm, leaf, meristem, root, seed, seedling, stem, and tassel libraries is used to identify cdps which are of interest.

VII. Extension of cDNA Sequences

The nucleic acid sequence was extended using an Incyte cDNA clone and oligonucleotide primers. One primer was synthesized to initiate 5′ extension of the known fragment, and the other, to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected plant cDNA libraries were used to extend the sequence. If more than one extension was necessary or desired, additional or nested sets of primers were designed. Preferred libraries are ones that have been size-selected to include larger cDNAs. Also, random primed libraries are preferred because they will contain more sequences with the 5′ and upstream regions of genes. A randomly primed library may be particularly useful if an oligo d(T) library does not yield a full-length cDNA. Genomic libraries are useful for extension 5′ of the promoter binding region in obtaining regulatory elements.

High fidelity amplification was obtained by PCR using methods well known in the art. PCR was performed in 96-well plates using the PTC-200 cycler (M J Research). The reaction mix contained DNA template, 200 nmol of each primer, reaction buffer containing Mg²⁺, (NH₄)₂SO₄, and β-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% v/v; Molecular Probes) dissolved in 1× TE and 0.5 μl of undiluted PCR product into each well of an opaque fluorimeter plate (Corning Costar, Acton Mass.) and allowing the DNA to bind to the reagent. The plate was scanned in a Fluoroskan II (Labsystems Oy) to measure the fluorescence of the sample and to quantify the concentration of DNA. A 5 μl to 10 μl aliquot of the reaction mixture was analyzed by electrophoresis on a 1% agarose mini-gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC18 vector (Amersham Pharmacia Biotech). For shotgun sequencing, the digested nucleotides were separated on low concentration (0.6 to 0.8%) agarose gels, fragments were excised, and agar digested with AGARACE enzyme (Promega, Madison Wis.). Extended clones were relegated using T4 DNA ligase (New England Biolabs, Beverly Mass.) into pUC18 vector (Amersham Pharmacia Biotech), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carbenicillin liquid media.

The cells were lysed, and DNA was amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified using PICOGREEN reagent (Molecular Probes) as described above. Samples with low DNA recoveries were reamplified using the same conditions described above. Samples were diluted with 20% dimethylsulfoxide (DMSO) (1:2, v/v), and sequenced using DYENAMIC energy transfer sequencing primers and the DYENAMIC DIRECT cycle sequencing kit (Amersham Pharmacia Biotech) or the ABI PRISM BIGDYE Terminator cycle sequencing kit (Perkin-Elmer).

VIII Labeling of Probes and Hybridization Analyses

Blotting

Target nucleic acids are isolated from a biological source and applied to a solid matrix (a blot) suitable for standard nucleic acid hybridization protocols by one of the following methods. A mixture of target nucleic acids, a restriction digest of genomic DNA, is fractionated by electrophoresis through an 0.7% agarose gel in 1× TAE (40 mM Tris acetate, ˜pH 8.5, 2 mM EDTA) running buffer and transferred to a nylon membrane by capillary transfer using 20× saline sodium citrate (SSC). Alternatively, the target nucleic acids are individually ligated to a vector and inserted into bacterial host cells to form a library. Target nucleic acids are then arranged on a blot by one of the following methods. In the first method, bacterial cells containing individual clones are robotically picked and arranged on a nylon membrane. The membrane is placed on bacterial growth medium, LB agar containing carbenicillin, and incubated at 37° C. for 16 hours. Bacterial colonies are denatured, neutralized, and digested with proteinase K. Nylon membranes are exposed to UV irradiation in a STRATALINKER UV-crosslinker (Stratagene) to cross-link DNA to the membrane.

In the second method, target nucleic acids are amplified from bacterial cells by thirty cycles of PCR using primers complementary to vector sequences flanking the insert. Amplified target nucleic acids are purified using SEPHACRYL-400 (Amersham Pharmacia Biotech). Purified target nucleic acids are robotically arrayed onto a glass microscope slide (Corning Science Products, Corning N.Y.). The slide was previously coated with 0.05% aminopropyl silane (Sigma-Aldrich, St. Louis Mo.) and cured at 110° C. The arrayed glass slide (microarray) is exposed to UV irradiation in a STRATALINKER UV-crosslinker (Stratagene).

Probe Preparation

cDNA probes are made from mRNA templates. Five micrograms of mRNA is mixed with 1 μg random primer (Life Technologies), incubated at 70° C. for 10 minutes, and lyophilized. The lyophilized sample is resuspended in 50 μl of 1× first strand buffer (cDNA synthesis system; Life Technologies) containing a dNTP mix, [α-³²P]dCTP, dithiothreitol, and MMLV reverse transcriptase (Stratagene), and incubated at 42° C. for 1-2 hours. After incubation, the probe is diluted with 42 μl dH₂O, heated to 95° C. for 3 minutes, and cooled on ice. mRNA in the probe is removed by alkaline degradation. The probe is neutralized, and degraded mRNA and unincorporated nucleotides are removed using a PROBEQUANT G-50 Micro Column (Amersham Pharmacia Biotech). Probes can be labeled with fluorescent markers, Cy3-dCTP or Cy5-dCTP (Amersham Pharmacia Biotech), in place of the radionuclide [³²P]dCTP.

Hybridization

Hybridization is carried out at 65° C. in a hybridization buffer containing 0.5 M sodium phosphate (pH 7.2), 7% SDS, and 1 mM EDTA. After the blot is incubated in hybridization buffer at 65° C. for at least 2 hours, the buffer is replaced with 10 ml of fresh buffer containing the probe. After incubation at 65° C. for 18 hours, the hybridization buffer is removed, and the blot is washed sequentially under increasingly stringent conditions, up to 40 mM sodium phosphate, 1% SDS, 1 mM EDTA at 65° C. To detect signal produced by a radiolabeled probe hybridized on a membrane, the blot is exposed to a PHOSPHORIMAGER cassette (Molecular Dynamics, Sunnyvale Calif.), and the image is analyzed using IMAGEQUANT data analysis software (Molecular Dynamics). To detect signals produced by a fluorescent probe hybridized on a microarray, the blot is examined by confocal laser microscopy, and images are collected and analyzed using GEMTOOLS gene expression analysis software (Incyte Pharmaceuticals).

X Restriction Fragment Length Polymorphisms

Restriction fragment length polymorphisms (RFLPs) are created by using one or more restriction enzymes to cut DNA into fragments at specific recognition sites. The fragments and molecular size standards are separated using gel electrophoresis. Ethidium bromide staining is used to reveal the fragments under UV (260 nm) illumination. Fragment size differences between samples result from mutations or sequence rearrangements within restriction enzyme recognition sites. RFLP markers are selected by examining these differences (Paterson, A. H. et al. (1988) Nature 335:721-726). Alternative DNA fragments include RAPDs (Welsh, J. and McClelland, M. (1990) Nucleic Acids Res. 18:7213-7218), microsatellites (also called simple sequence repeats; Akkaya, M. S. et al. (1992) Genetics 132:1131-1139) or amplified fragment length polymorphisms (AFLPs; Nandi, S. et al. (1997); Mol. Gen. Genet. 255:1-8). Any of these DNA fragments may be mapped onto a chromosomal map, and all are chosen and used to study multigene traits or quantitative trait loci (QTL) at the intraspecific level or among closely related taxa.

XI Peptide Expression

Expression of a corn peptide is accomplished by transforming the multifunctional vector, PSPORT (Life Technologies) or pINCY (Incyte Pharmaceuticals), containing the sequence encoding the peptide into E. coli. A transfected colony is cultured and induced with isopropyl beta-D-thiogalactopyranoside (IPTG) using standard methods. The signal sequence resident in the vector directs the secretion of the peptide into the bacterial growth media. Purification of the peptide using polyacrylamide gel electrophoresis will provide peptide for antibody induction or for use in various assays.

XII Production of Antibodies

The amino acid sequence encoded by a cdp is analyzed using LASERGENE Navigator (DNASTAR) to determine regions of high immunogenicity. An oligopeptide of about 15 residues is synthesized using a Peptide Synthesizer Model 431A (Perkin Elmer) using Fmoc-chemistry and coupled to KLH (Sigma-Aldrich) by reaction with M-maleimidobenzoyl-N-hydroxysuccinimide ester (Ausubel, supra) . If necessary, a cysteine may be introduced at the N-terminus of the oligopeptide to permit coupling to KLH. Rabbits are immunized with the oligopeptide-KLH complex in complete Freund's adjuvant. The resulting antisera are tested for antipeptide activity, for example, by binding the peptide to plastic, blocking with 1% BSA, reacting with rabbit antisera, washing, and reacting with radioiodinated goat anti-rabbit IgG.

XIII Two-Hybrid Screen

A yeast two-hybrid system such as MATCHMAKER LexA Two-Hybrid system (Clontech Laboratories, Palo Alto Calif.) is used to screen for peptides which bind CDPs. A nucleotide encoding a CDP is inserted into the multiple cloning site of a pLexA vector, ligated, and transformed into E. coli. cDNA, prepared from mRNA, is directionally inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48[p8op-lacZ] using a polyethylene glycol/lithium acetate protocol. The transformed yeast cells are plated on synthetic dropout (SD) media lacking histidine (-His), tryptophan (-Trp), and uracil (-Ura), and incubated at 30° C. until colonies may be easily counted. The colonies are pooled in a minimal volume of 1× TE (pH 7.5), replated on SD/-His/-Leu/-Trp/-Ura media supplemented with 2% galactose (Gal), 1% raffinose (Raf), and 80 mg/ml X-Gal (X-Gal), and subsequently examined for growth of blue colonies. Interaction between expressed CDP and cDNA fusion proteins activates expression of a LEU2 reporter gene in EGY48 and produces colony growth on media lacking leucine (-Leu). Interaction also activates expression of β-galactosidase from the p8op-lacZ reporter construct which produces blue color in colonies grown on X-Gal.

Positive interactions between expressed CDP and cDNA fusion proteins can be verified by isolating individual positive colonies and growing them in SD/-Trp/-Ura liquid medium for 1-2 days at 30° C. A sample of the culture is plated on SD/-Trp/-Ura media and incubated at 30° C. until colonies appear. A sample of 20-30 colonies is identically arranged on SD/-Trp/-Ura and SD/-His/-Trp/-Ura plates. Colonies that grow on SD containing histidine but not on media lacking histidine have lost the pLexA plasmid. The histidine-requiring colonies are grown on SD/Gal/Raf/X-Gal/-Trp/-Ura, and white colonies are isolated and propagated. The pB42AD-cDNA plasmid, which contains a polynucleotide encoding a protein which physically interacts with a CDP, can be isolated from the yeast cells and characterized.

XIV Significant Sequences and Their Uses

The biological activity of polypeptides encoded by the cdps is based in part on a comparison between nucleic acid sequences in the Sequence Listing and reference or homologous sequences from GenBank which encode polypeptides of known function or activity. The biological properties and potential uses of polypeptides encoded by the cdps are based in part upon the biological properties of their known homologs.

Incyte Clone No. 700345457H1 is a nonexact homolog of GenBank GI No. g939780, which encodes ZAG5, a MADS-box protein in maize (Mena, M. et al. (1995) Plant J. 8:845-854).

The MADS domain (MCM1, AGAMOUS, IDEFICIENS, and SRF, serum response factor) is a conserved DNA-binding/dimerization region present in a variety of transcription factors from different kingdoms. MADS box genes represent a large multigene family in vascular plants. In angiosperms, many of the genes of the MADS family are involved in different steps of flower development, most notably in the determination of floral meristem and organ identity (Riechmann, J. L. and Meyerowitz, E. M. (1997) Biol Chem 378:1079-101).

As part of an effort to understand how such genes may have been recruited during the evolution of flowers two members of this gene family, ZAG1 and ZAG2, were characterized in maize (Schmidt, R. J. et al. (1993) Plant Cell 7:729-737). In a subsequent study, Mena, M. et al. described the isolation and characterization of four new members of the family, designated ZAP1, ZAG3, ZAG4 and ZAG5. They mapped these four and 28 additional maize MADS-box genes. The first new member of this family, designated ZAP1, appeared to be the Zea mays ortholog of the floral homeotic gene APETALA1 (AP1). The deduced protein sequence of one of these genes, ZAG4, included the MADS domain but lacked the K-domain characteristically present in this family. In addition, copy number and expression of ZAG4 varied among different inbred lines. A large number of maize MADS-box genes, including ZAG3 and ZAG5, map to duplicate regions of the genome. These data underscore the complexity of the MADS-box family in maize and of the regulation of floral organ morphogenesis among the grasses.

Incyte Clone No. 700345457H1 is defined as a nonexact but functionally related homolog based on a product score of 20 and a log-likelihood value of −13 as shown in TABLE 1. Incyte Clone No. 700345457H1 can be used as a marker to follow inheritance in a corn breeding program, as an element in an array to monitor the effects of the application of agrichemicals, or as a tool to investigate the regulation of floral morphogenesis.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

TABLE 1
LIST OF INCYTE CLONES DERIVED FROM SATMON021024
COPYRIGHT 1998 INCYTE PHARMACEUTICALS, INC.

| | | | | | | |
|---|---|---|---|---|---|---|
| 700345225H1 | g683501 | 28 | −9 | gb105pln | A. thaliana mRNA for 65 kDa regulatory subunit of protein phosphatase 2A. | |
| 700343709H1 | g167109 | 30 | −12 | gb105pln | Hordeum vulgare vacuolar ATPase B subunit mRNA, complete cds. | |
| 700343622H1 | g1658314 | 26 | −2 | gb105pln | O. sativa osr40g3 gene. | |
| 700342637H1 | g1777386 | 9 | 7 | gb105allp | caffeic acid O-methyltransferase | |
| 700342455H1 | g2668743 | 63 | −46 | gb105pln | Zea mays ubiquitin conjugating enzyme (UBC) mRNA, complete cds. | |
| 700346284H1 | g1546918 | 34 | −62 | gb105pln | Z. mays mRNA for translation initiation factor 5A. | |
| 700344743H1 | g198576 | 8 | 3 | gb105allp | hydrophilic protein | |
| 700346601H1 | g1556430 | 40 | −32 | gb105pln | Populus hybrid mRNA for citrate synthase. | |
| 700343489H1 | g998429 | 88 | −55 | gb105pln | GRF1 = general regulatory factor [Zea mays, XL80, Genomic, 5348 nt]. | |
| 700342946H1 | g218241 | 20 | −34 | gb105pln | Rice mRNA for ribosomal protein L3 (T82 gene), partial sequence. | |
| 700344014H1 | g2829882 | 55 | 0 | gb105eukp | F3I6.30; beta-oxoacyl-(acyl carrier protein) reductase; EC 1.1.1.100 | |
| 700342243H1 | g2244837 | 21 | 1 | gb105allp | proteasome chain protein | |
| 700346337H1 | g473986 | 15 | −13 | gb105pln | Rice mRNA, partial homologous to histone H2B gene. | |
| 700343215H1 | g2662346 | 60 | −43 | gb105pln | Oryza sativa mRNA for EF-1 alpha, complete cds. | |
| 700344166H1 | g728698 | 20 | −3 | gb105pln | Yeast (Saccharomyces cerevisiae) DNA from left arm of chromosome X. | |
| 700346020H1 | g1370155 | 22 | −7 | gb105pln | L. japonicus mRNA for small GTP-binding protein, RAB11H. | |
| 700345572H1 | g303852 | 43 | −53 | gb105pln | Rice mRNA for ribosomal protein L3, complete cds. | |
| 700346091H1 | g1235572 | 34 | −20 | gb105pln | P. curvicolla EF1-alpha gene. | |
| 700379754H1 | g1922277 | 24 | 13 | gb105pln | Fragaria x ananassa mRNA for TCTP protein. | |
| 700345550H1 | g2832664 | 7 | 7 | gb105eukp | F28J12.250; pollen-specific protein-like | |
| 700344542H1 | g2662153 | 10 | 5 | gb105allp | KIAA0436 | |
| 700344387H1 | g1871360 | 13 | −3 | gb105eukp | glutamyl-prolyl-tRNA synthetase | |
| 700343710H1 | g1184773 | 62 | −54 | gb105pln | Zea mays glyceraldehyde-3-phosphate dehydrogenase GAPC3 (gpc3) mRNA, | |
| | | | | | complete cds. | |
| 700346408H1 | g395071 | 18 | −9 | gb105pln | V. faba guanine nucleotide regulatory protein mRNA, complete CDS. | |
| 700344952H1 | g313266 | 36 | −19 | gb105pln | T. aestivum gene for phosphoglycerate kinase. | |
| 700345682H1 | g300082 | 48 | −4 | gb105pln | hsp82 = 82 kda heat shock protein [Zea mays, seedling, leaves, Genomic, 3468 nt]. | |
| 700344543H1 | g2330817 | 27 | −5 | gb105pln | Yeast (Saccharomyces pombe) chromosome I cosmid c4F8. | |
| 700343777H1 | g2244834 | 42 | −18 | gb105eukp | hypothetical protein | |
| 700342769H1 | g1788865 | 6 | 2 | gb105allp | hypothetical 43.1 kD protein in ndk-gcpE intergenic region | |
| 700380696H1 | g602252 | 100 | −87 | gb105pln | Zea mays enolase (eno2) mRNA, complete cds. | |
| 700346044H1 | g2808637 | 31 | −17 | gb105pln | Daucus carota mRNA for Rab8-like small GTP-binding protein. | |
| 700380075H1 | g21800 | 19 | −24 | gb105pln | T. aestivum L mRNA for histone H2B. | |
| 700344370H1 | g16291 | 13 | 12 | gb105pln | A. thaliana mRNA for glycine-rich RNA-binding protein. | |
| 700281843H2 | g507825 | 11 | 7 | gb105eukp | Putrescine N-Methyltransferase; EC 2.1.1.53 | |
| 700345821H1 | g1196896 | 44 | −30 | gb105pln | Glycine max acidic ribosomal protein P0 mRNA, complete cds. | |
| 700345723H1 | g1498052 | 72 | −82 | gb105pln | Zea mays ribosomal protein S8 mRNA, complete cds. | |
| 700346083H1 | g218132 | 10 | 16 | gb105pln | Rice mRNA for Heat shock protein 83. | |
| 700345836H1 | g644491 | 73 | −57 | gb105pln | Corn mRNA for elongation factor 1A. | |
| 700345431H1 | g551482 | 14 | −1 | gb105pln | Zea mays ABA- and ripening-inducible-like protein mRNA, complete cds. | |
| 700346904H1 | g780371 | 67 | −57 | gb105pln | Oryza sativa enolase mRNA, complete cds. | |
| 700346902H1 | g790507 | 60 | −45 | gb105pln | Z. mays mRNA for 60S acidic ribosomal protein. | |
| 700346162H1 | g642130 | 11 | −4 | gb105eukp | AME1; protein kinase | |
| 700345439H1 | g173231 | 18 | 3 | gb105pln | Yeast (Saccharomyces cerevisiae) ribosomal 5S RNA-binding protein (YL3) gene, 5′ cds. | |
| 700346889H1 | g829221 | 27 | −1 | gb105eukp | histone H2 A.F/Z | |
| 700344102H1 | g1001532 | 40 | −17 | gb105allp | hypothetical protein | |
| 700379931H1 | g485952 | 51 | −40 | gb105pln | O. sativa mRNA for glutaredoxin. | |
| 700346181H1 | g170746 | 48 | 14 | gb105pln | Wheat histone H4 TH091 gene, complete cds. | |
| 700345518H1 | g20237 | 18 | −2 | gb105pln | O. sativa (rice) constitutive GOS2 gene. | |
| 700347133H1 | g2149051 | 51 | 0 | gb105eukp | Ran3; small Ras-like GTP-binding protein | |
| 700344902H1 | g600386 | 28 | −15 | gb105pln | A. thaliana mRNA for proteasome subunit. | |
| 700380685H1 | g2529668 | 8 | 3 | gb105eukp | T30B22.11; putative photolyase/blue-light receptor | |
| 700343763H1 | g2145358 | 9 | −1 | gb105eukp | ATHB-9; transcription factor; HD-Zip protein | |
| 700342558H1 | g2444276 | 53 | −46 | gb105pln | Arabidopsis thaliana GSK3/shaggy-like protein kinase (GSK1) mRNA, complete cds. | |
| 700379996H1 | g312732 | 15 | 4 | gb105allp | beta prime cop | |
| 700379841H1 | g2462760 | 11 | 6 | gb105eukp | F8A5.19 | |
| 700346223H1 | g2264319 | 20 | −2 | gb105pln | Arabidopsis thaliana genomic DNA, chromosome 5, P1 clone: MXA21, | |
| | | | | | complete sequence. | |
| 700345766H1 | g4590 | 30 | 1 | gb105pln | Yeast (Saccharomyces cerevisiae) SUG1 gene. | |
| 700343339H1 | g2425030 | 17 | 5 | gb105allp | perchloric acid soluble protein | |
| 700380162H1 | g2662344 | 41 | −31 | gb105pln | Oryza sativa mRNA for EF-1 alpha, complete cds. | |
| 700345123H1 | g2599103 | 19 | 1 | gb105pln | Dunaliella salina 60S ribosomal protein (DSRP1) mRNA, complete cds. | |
| 700343289H1 | g2651294 | 16 | 13 | gb105pln | Arabidopsis thaliana chromosome II BAC T2P4 genomic sequence, complete sequence. | |
| 700344384H1 | g2464894 | 13 | 13 | gb105pln | Arabidopsis thaliana DNA chromosome 4, ESSA I AP2 contig fragment No. 2. | |
| 700343363H1 | g2791947 | 14 | 15 | gb105pln | Lupinus luteus mRNA for ribosomal protein L13a. | |
| 700380284H1 | g432367 | 28 | −11 | gb105pln | Rice mRNA for elongation factor 1 beta, complete cds. | |
| 700346456H1 | g312602 | 17 | 15 | gb105pln | P. obtusangulum 26S rRNA (partial). | |
| 700343433H1 | g559395 | 16 | −2 | gb105eukp | dihydrolipoamide acetyltransferase (E2) subunit of PDC | |
| 700344302H1 | g21800 | 20 | −28 | gb105pln | T. aestivum L mRNA for histone H2B. | |
| 700343113H1 | g2351070 | 37 | −16 | gb105pln | Arabidopsis thaliana genomic DNA, chromosome 5, P1 clone: MTH12. | |
| 700347238H1 | g553063 | 21 | −4 | gb105eukp | GST2; glutathione s-transferase | |
| 700344486H1 | g886024 | 10 | 6 | gb105allp | protein kinase PkwA | |
| 700342307H1 | g602398 | 30 | −15 | gb105eukp | SPF1; P-type ATPase | |
| 700343331H1 | g1495259 | 15 | 1 | gb105allp | orf04 | |
| 700344032H1 | g2244928 | 32 | 6 | gb105eukp | hypothetical protein | |
| 700346601H1 | g1556379 | 36 | −27 | gb105pln | B. vulgaris mRNA for citrate synthase. | |
| 700344971H1 | g304108 | 23 | −2 | gb105pln | Arabidopsis thaliana poly(A)-binding protein mRNA, complete cds. | |
| 700345170H1 | g1107460 | 28 | −12 | gb105pln | Rice mRNA for aspartate kinase-homoserine dehydrogenase, complete cds. | |
| 700343996H1 | g435542 | 78 | −75 | gb105pln | Z. mays mRNA for calmodulin. | |
| 700342952H1 | g1458096 | 16 | 1 | gb105allp | enterocyte differentiation promoting factor | |
| 700342334H1 | g452473 | 86 | −72 | gb105pln | Zea mays Black Mexican Sweet alpha-tubulin mRNA, complete cds. | |
| 700344212H1 | g2651294 | 15 | 3 | gb105pln | Arabidopsis thaliana chromosome II BAC T2P4 genomic sequence, complete sequence. | |
| 700343661H1 | g960356 | 25 | −15 | gb105pln | Barley mRNA for S-adenosylmethionine sythetase, complete cds. | |
| 700379966H1 | g19500 | 44 | −36 | gb105pln | Lupinus polyphyllus lupin-specific pPLZ12 mRNA. | |
| 700343384H1 | g168421 | 77 | −59 | gb105pln | Maize auxin-binding protein (ABP) mRNA, complete cds. | |
| 700342705H1 | g1778373 | 22 | −11 | gb105pln | Pisum sativum glycine-rich RNA-binding protein PsGRBP mRNA, complete cds. | |
| 700345876H1 | g2244767 | 22 | −6 | gb105eukp | COP9 protein | |
| 700343719H1 | g1246841 | 30 | −12 | gb105eukp | G2827 | |
| 700347294H1 | g1184771 | 82 | −79 | gb105pln | Zea mays glyceraldehyde-3-phosphate dehydrogenase GAPC2 (gpc2) mRNA, | |
| | | | | | complete cds. | |
| 700344632H1 | g488198 | 20 | −3 | gb105eukp | ZC155.1 | |
| 700346915H1 | g1147599 | 7 | 4 | gb105eukp | dod; dodo | |
| 700345244H1 | g1916867 | 11 | 6 | gb105eukp | who; muscle development; WHO | |
| 700342529H1 | g22324 | 69 | −55 | gb105pln | Z. mays mRNA for H2B histone (clone cH2B221). | |
| 700380634H1 | g2306767 | 16 | 6 | gb105pln | Triticum aestivum eIF-2 beta subunit mRNA, complete cds. | |
| 700344884H1 | g289658 | 46 | −27 | gb105eukp | C50C3.6 protein | |
| 700347051H1 | g170343 | 12 | 4 | gb105pln | Nicotiana tabacum translation elongation factor EF-Tu gene, complete cds. | |
| 700380670H1 | g644492 | 58 | −58 | gb105pln | Corn elongation factor 1alpha gene, complete cds. | |
| 700342672H1 | g1322276 | 23 | −10 | gb105pln | Triticum aestivum histone H2A gene, complete cds. | |
| 700343564H1 | g1167953 | 8 | −1 | gb105eukp | putative 32.6 kDa jasmonate-induced protein | |
| 700380110H1 | g1397320 | 23 | −8 | gb105eukp | R08C7.3 | |
| 700346207H1 | g304153 | 13 | 3 | gb105allp | sorbitol dehydrogenase | |
| 700345346H1 | g529355 | 10 | 5 | gb105eukp | T12A2.4 | |
| 700342489H1 | g2662536 | 12 | 2 | gb105eukp | C05B10.1 | |
| 700346464H1 | g2081612 | 13 | 5 | gb105allp | deltal-pyrroline-5-carboxylate synthetase | |
| 700342934H1 | g2196671 | 62 | −49 | gb105pln | Z. mays mRNA for HMG protein. | |
| 700380180H1 | g1731990 | 21 | −11 | gb105eukp | Cxp; 2; serine carboxypeptidase II, CP-MII | |
| 700347227H1 | g451193 | 15 | −9 | gb105eukp | wali7 | |
| 700345819H1 | g1899026 | 24 | −35 | gb105pln | Zea mays superoxide dismutase 4A (sod4A) gene, complete cds. | |
| 700344370H1 | g2624327 | 35 | −22 | gb105pln | Oryza sativa mRNA for glycine rich RNA-binding protein 2 (OsGRP2). | |
| 700380434H1 | g166858 | 9 | −1 | gb105eukp | ribosomal protein | |
| 700344794H1 | g22328 | 31 | −24 | gb105pln | Maize mRNA for a high mobility group protein. | |
| 700347351H1 | g2341060 | 44 | −62 | gb105pln | Zea mays translational initiation factor eIF-4A (tif-4A3) mRNA, complete cds. | |
| 700344019H1 | g1737219 | 35 | −27 | gb105pln | Arabidopsis thaliana vacuolar sorting receptor homolog mRNA, complete cds. | |
| 700345876H1 | g530870 | 22 | −6 | gb105eukp | COP9 | |
| 700342531H1 | g1335861 | 35 | −20 | gb105pln | Glycine max clathrin heavy chain mRNA, complete cds. | |
| 700346048H1 | g304037 | 6 | 6 | gb105eukp | ENOD8; early nodulin | |
| 700343030H1 | g498774 | 42 | −47 | gb105pln | Z. mays (cv DH5xDH7) hsp70-5 mRNA for heat shock protein 70. | |
| 700342952H1 | g2689608 | 16 | 0 | gb105allp | UEV1Bs | |
| 700347238H1 | g167970 | 18 | −0 | gb105eukp | GST1 | |
| 700380563H1 | g2274983 | 26 | −32 | gb105pln | Hordeum vulgare mRNA for expressed sequence tag. | |
| 700380216H1 | g288062 | 30 | −22 | gb105pln | A. thaliana mRNA for ketol-acid reductoisomerase subunit. | |
| 700343406H1 | g939778 | 28 | −20 | gb105pln | Zea mays MADS-box protein (ZAG3) mRNA, complete cds. | |
| 700346201H1 | g22237 | 70 | −85 | gb105pln | Maize mRNA for cytosolic GAPDH (GapC) glyceraldehyde-3-phosphate dehydrogenase. | |
| 700343843H1 | g2330856 | 19 | −3 | gb105eukp | SPAC6F12.09; hypothetical protein | |
| 700343828H1 | g747916 | 98 | −86 | gb105pln | Z. mays CaM2 mRNA for calmodulin. | |
| 700346135H1 | g1136422 | 59 | −28 | gb105allp | KIAA0181 | |
| 700343652H1 | g493622 | 28 | −9 | gb105eukp | ACA1; chloroplast envelope Ca2+−ATPase precursor | |
| 700344145H1 | g169818 | 39 | −55 | gb105pln | Rice 25S ribosomal RNA gene. | |
| 700343953H1 | g968901 | 35 | −43 | gb105pln | Rice mRNA for ribosomal protein S8, complete cds. | |
| 700347075H1 | g2264302 | 11 | 14 | gb105pln | Arabidopsis thaliana genomic DNA, chromosome 5, P1 clone: MAC12, | |
| | | | | | complete sequence. | |
| 700342483H1 | g1066996 | 34 | −19 | gb105eukp | K03H1.2 | |
| 700344248H1 | g2708314 | 30 | −12 | gb105eukp | PDI; involved in the redox-regulated binding of chloroplast translational | |
| | | | | | activation; protein disulfide isomerase RB60; EC 5.3.4.1 | |
| 700346091H1 | g1136786 | 34 | −20 | gb105pln | Fission Yeast mRNA for elongation factor 1 alpha-C, complete cds. | |
| 700344358H1 | g2262105 | 21 | −8 | gb105eukp | T19F06.8 | |
| 700344220H1 | g22116 | 60 | −66 | gb105pln | Z. mays ZMACK2 mRNA for casein kinase II alpha subunit. | |
| 700342577H1 | g1405747 | 17 | −0 | gb105allp | PR264/SC35 | |
| 700380144H1 | g1881692 | 80 | −74 | gb105pln | Zea mays phosphoglucomutase mRNA, partial cds. | |
| 700343888H1 | g172559 | 11 | −4 | gb105eukp | Sec13; Sec13p | |
| 700346688H1 | g732814 | 48 | −33 | gb105pln | L. esculentum LeMA-1 mRNA for putative Mg-dependent ATPase 1. | |
| 700380653H1 | g168500 | 44 | −34 | gb105pln | Maize (Zea mays) histone H4 gene (H4C14), complete cds. | |
| 700342744H1 | g2791947 | 22 | −3 | gb105pln | Lupinus luteus mRNA for ribosomal protein L13a. | |
| 700344884H1 | g395157 | 6 | 7 | gb105eukp | PRP8; pre-mRNA splicing factor, U5 snRNP protein; PRP8 | |
| 700342583H1 | g1432053 | 56 | −45 | gb105pln | Oryza sativa asparagine synthetase mRNA, complete cds. | |
| 700347063H1 | g603808 | 38 | −24 | gb105eukp | STT3; STT3 protein | |
| 700345264H1 | g2706449 | 33 | −17 | gb105pln | Solanum tuberosum mRNA for magnesium dependent soluble inorganic pyrophosphatase. | |
| 700380449H1 | g1665777 | 8 | 7 | gb105allp | Similar to S. cerevisiae EMP70 protein precursor (S25110) | |
| 700347240H1 | g218088 | 59 | −61 | gb105pln | Rice mRNA for ribosomal protein 117 (249 gene), partial sequence. | |
| 700343328H1 | g2191188 | 28 | −1 | gb105eukp | A_TM021B04.12 | |
| 700345093H1 | g951335 | 75 | −65 | gb105pln | Oryza sativa protein phosphatase 1 mRNA, complete cds. | |
| 700380665H1 | g2145472 | 23 | −8 | gb105pln | S. tuberosum mRNA for aconitase/aconitate hydratase. | |
| 700347024H1 | g22106 | 74 | −67 | gb107pln | Maize mRNA for putative auxin binding protein. | |
| 700344314H1 | g2160155 | 32 | −19 | gb105pln | Sequence of BAC F21M12 from Arabidopsis thaliana chromosome 1, complete | |
| | | | | | sequence. | |
| 700344283H1 | g1022804 | 48 | −31 | gb105pln | Arabidopsis thaliana phosphoglycerate kinase mRNA, partial cds. | |
| 700379794H1 | g22324 | 43 | −4 | gb105pln | Z. mays mRNA for H2B histone (clone cH2B221). | |
| 700344273H1 | g1388084 | 14 | −5 | gb105eukp | TRX5; thioredoxin h | |
| 700343541H1 | g2832745 | 12 | 4 | gb105eukp | SPAC23H3.08c; hypothetical protein | |
| 700343041H1 | g450548 | 40 | −39 | gb105pln | O. sativa (pRSAM-1) gene for S-adenosyl methionine synthetase. | |
| 700342491H1 | g2108344 | 43 | −43 | gb105pln | Brassica campestris small GTP-binding protein Bsar1a (bsar1a) mRNA, complete cds. | |
| 700346851H1 | g313151 | 12 | 17 | gb105pln | A. thaliana ribosomal protein S15 mRNA, complete CDS. | |
| 700346436H1 | g557470 | 36 | 5 | gb105allp | similar to S. cerevisiae PMS1 Swiss-Prot Accession Number P14242, S. cerevisiae | |
| | | | | | MLH1 GenBank Accession Number U07187, E. coli MUTL Swiss-Prot Accession | |
| | | | | | Number P23367, Salmonella typhimurium MUTL Swiss-Prot Accession Number | |
| | | | | | P14161, Streptococcu> | |
| 700344740H1 | g303855 | 13 | −7 | gb105eukp | ribosomal protein L7A | |
| 700343377H1 | g825783 | 22 | 7 | gb105pln | Nicotiana tabacum ribosomal protein L41 mRNA, complete cds. | |
| 700345695H1 | g2662344 | 69 | −63 | gb105pln | Oryza sativa mRNA for EF-1 alpha, complete cds. | |
| 700281915H1 | g290275 | 30 | −5 | gb105eukp | M(3)95A; multifunctional activity and location; ribosomal protein S3/AP endonuclease | |
| | | | | | DNA repair protein | |
| 700347031H1 | g514945 | 96 | −42 | gb105pln | Zea mays sucrose synthase (Sus1) mRNA, complete cds. | |
| 700345418H1 | g1129084 | 27 | −11 | gb105pln | Wheat mRNA for protein H2A, complete cds, clone wcH2A-3. | |
| 700344337H1 | g2343181 | 19 | −6 | gb105eukp | MDL3; catalyzes dissociation of (R)-mandelonitrile to hydrogen cyanide and | |
| | | | | | benzaldehyde; (R)-(+)-mandelonitrile lyase isoform MDL3 precursor; EC 4.1.2.10 | |
| 700346017H1 | g2582061 | 65 | 2 | gb105allp | NADH dehydrogenase subunit 2 | |
| 700344147H1 | g2257480 | 21 | −7 | gb105pln | Yeast (Schizosaccharomyces pombe) 38 kb genomic DNA, clone 1750. | |
| 700343362H1 | g2109293 | 13 | 7 | gb105eukp | serine/threonine protein kinase | |
| 700342491H1 | g1549221 | 40 | −39 | gb105pln | Tobacco mRNA for NtSar1 protein, complete cds. | |
| 700342377H1 | g1008880 | 53 | −50 | gb105pln | Phaseolus vulgaris eukaryotic initiation factor 5 (eIF-5) gene, complete cds. | |
| 700342409H1 | g473604 | 87 | −80 | gb105pln | Zea mays W-22 histone H2B mRNA, complete cds. | |
| 700379682H1 | g2293565 | 31 | −13 | gb105pln | Oryza sativa ADP-ribosylation factor 1 (Os-ARF1) mRNA, complete cds. | |
| 700346263H1 | g1931636 | 16 | 1 | gb105pln | Arabidopsis thaliana BAC T19D16 genomic sequence. | |
| 700345614H1 | g2668747 | 56 | −49 | gb105pln | Zea mays ribosomal protein L17 (rp117) mRNA, complete cds. | |
| 700346435H1 | g1931655 | 7 | −1 | gb105eukp | T19D16.21; receptor-kinase isolog | |
| 700380548H1 | g1254995 | 19 | −1 | gb105pln | Arabidopsis thaliana serine/threonine protein phosphatase type 2A regulatory | |
| | | | | | subunit A mRNA, complete cds. | |
| 700344851H1 | g577818 | 14 | 1 | gb105pln | Z. mays gene for H2B histone (gH2B4). | |
| 700346944H1 | g927323 | 30 | −11 | gb105eukp | UBA2; Uba2p | |
| 700345948H1 | g2262135 | 23 | −2 | gb105pln | Arabidopsis thaliana BAC T10P11, complete sequence. | |
| 700281952H2 | g169792 | 41 | −46 | gb105pln | Rice histone 3 gene, complete cds. | |
| 700346795H1 | g857573 | 36 | −23 | gb105pln | Oryza sativa vacuolar H+-ATPase (vatp-P1) mRNA, complete cds. | |
| 700343710H1 | g312180 | 32 | −57 | gb105pln | Z. mays GapC4 gene. | |
| 700344743H1 | g2648022 | 8 | 4 | gb105allp | HKE2 | |
| 700342888H1 | g999189 | 44 | −32 | gb105pln | dihydrofolate reductase-thymidylate synthase = bifunctional enzyme [Glycine max, | |
| | | | | | seedling, mRNA, 1794 nt]. | |
| 700342483H1 | g1439562 | 20 | −8 | gb105eukp | cdc28; RNA-dependent ATPase/helicase; Cdc28p | |
| 700346252H1 | g899609 | 57 | −66 | gb105pln | Zea mays acidic ribosomal protein P2 (RPA-2A1) mRNA, complete cds. | |
| 700343951H1 | g2828147 | 8 | 7 | gb105allp | BC-2 protein | |
| 700345044H1 | g902524 | 86 | −14 | gb105pln | Zea mays clone MubG10 ubiquitin fusion protein gene, complete cds. | |
| 700343352H1 | g169363 | 26 | −7 | gb105eukp | PVPR3 | |
| 700342479H1 | g309418 | 17 | −4 | gb105allp | androgen-regulated protein | |
| 700346501H1 | g1129083 | 33 | −21 | gb105pln | Wheat mRNA for protein H2A, complete cds, clone wcH2A-2. | |
| 700342216H1 | g313135 | 24 | −4 | gb105pln | Z. mays mRNA for porin. | |
| 700344655H1 | g2394226 | 21 | 15 | gb105pln | Lycopersicon esculentum WD-40 repeat protein (LeMSI1) mRNA, complete cds. | |
| 700344162H1 | g2511530 | 21 | 5 | gb105pln | Eleusine indica alpha tubulin 1 (TUA1) mRNA, complete cds. | |
| 700347350H1 | g173231 | 18 | 2 | gb105pln | Yeast (Saccharomyces cerevisiae) ribosomal 5S RNA-binding protein (YL3) gene, 5′ cds. | |
| 700346607H1 | g2408094 | 8 | 2 | gb105allp | hypothetical protein | |
| 700342376H1 | g490094 | 62 | −34 | gb105allp | TIMP gene product | |
| 700344736H1 | g1203905 | 33 | −316 | gb105eukp | M(1)15D; M(1)15D | |
| 700346634H1 | g218088 | 38 | −28 | gb105pln | Rice mRNA for ribosomal protein 117 (249 gene), partial sequence. | |
| 700345391H1 | g170703 | 32 | −42 | gb105pln | Wheat gibberellin responsive protein gene, complete cds. | |
| 700344903H1 | g172235 | 12 | −2 | gb105eukp | PR12 | |
| 700343202H1 | g19867 | 6 | 7 | gb105eukp | extension (AA 1-620) | |
| 700342267H1 | g1184773 | 96 | −84 | gb105pln | Zea mays glyceraldehyde-3-phosphate dehydrogenase GAPC3 (gpc3) mRNA, | |
| | | | | | complete cds. | |
| 700346058H1 | g790507 | 91 | −80 | gb105pln | Z. mays mRNA for 60S acidic ribosomal protein. | |
| 700346017H1 | g2052363 | 65 | 2 | gb105allp | protein 2 | |
| 700345943H1 | g2582413 | 14 | 3 | gb105allp | STE20-like kinase 3 | |
| 700345637H1 | g172423 | 23 | −6 | gb105pln | Yeast (Saccharomyces cerevisiae) ribosomal protein L1 gene, complete cds. | |
| 700345286H1 | g22292 | 88 | −73: | gb105pln | Z. mays mRNA for glycine-rich protein. | |
| 700343655H1 | g463856 | 32 | −23 | gb105pln | Chlamydomonas reinhardtii 21 gr ribosomal protein S14 (CRY1) gene, complete cds. | |
| 700379970H1 | g170746 | 40 | −6 | gb105pln | Wheat histone H4 TH091 gene, complete cds. | |
| 700345462H1 | g311906 | 29 | −16 | gb105pln | N. tabacum mRNA for ras-related GTP-binding protein. | |
| 700346541H1 | g488574 | 29 | —13 | gb105pln | Medicago sativa Regen S clone pH3c130 histone H3.2 mRNA, complete cds. | |
| 700346446H1 | g17738 | 16 | −4 | gb105eukp | beta-1,3-glucanase homologue | |
| 700346196H1 | g1136119 | 77 | −64 | gb105pln | O. sativa mRNA for alpha-tubulin (clone OSTA-111). | |
| 700344859H1 | g2351560 | 11 | 8 | gb105allp | spindle assembly checkpoint protein Mad2p | |
| 700343858H1 | g1857256 | 10 | 4 | gb105eukp | mudrAzc; MURAZC | |
| 700380093H1 | g1045304 | 26 | −20 | gb105pln | Zea mays acetyl-coenzyme A carboxylase mRNA, complete cds. | |
| 700342577H1 | g455419 | 17 | 1 | gb105allp | PR264/SC35 | |
| 700346952H1 | g2760173 | 18 | −3 | gb105pln | Arabidopsis thaliana genomic DNA, chromosome 5, P1 clone: MYH19, complete | |
| | | | | | sequence. | |
| 700345168H1 | g2467274 | 8 | 6 | gb105eukp | SPBC16E9.12c; rna binding protein | |
| 700344443H1 | g2641637 | 10 | 16 | gb105pln | Arabidopsis thaliana DnaJ homolog AtJ3 (ATJ3) gene, complete cds. | |
| 700344032H1 | g2245031 | 22 | 17 | gb105pln | Arabidopsis thaliana DNA chromosome 4, ESSA I contig fragment No. 7. | |
| 700379782H1 | g2264307 | 17 | 14 | gb105pln | Arabidopsis thaliana genomic DNA, chromosome 5, P1 clone: MED24, | |
| | | | | | complete sequence. | |
| 700346305H1 | g287628 | 48 | −8 | gb105eukp | YS29A; ribosomal protein YS29 | |
| 700346718H1 | g19280 | 40 | −32 | gb105pln | L. esculentum mRNA for enolase. | |
| 700342420H1 | g169792 | 32 | −44 | gb105pln | Rice histone 3 gene, complete cds. | |
| 700346066H1 | g976256 | 57 | −45 | gb105pln | Rice mRNA stearyl-ACP desaturase, complete cds. | |
| 700344527H1 | g872116 | 29 | −8 | gb105eukp | sti; sti (stress inducible protein) | |
| 700343316H1 | g163103 | 45 | −21 | gb105allp | glutaminyl cyclase | |
| 700343921H1 | g2828278 | 18 | −16 | gb105pln | Arabidopsis thaliana DNA chromosome 4, BAC clone T18B16 (ESSAII project). | |
| 700344193H1 | g204070 | 14 | 5 | gb105allp | electron transfer flavoprotein alpha-subunit | |
| 700342640H1 | g157944 | 43 | −19 | gb105eukp | Mhc | |
| 700379925H1 | g2264302 | 10 | 16 | gb105pln | Arabidopsis thaliana genomic DNA, chromosome 5, P1 clone: MAC12, complete | |
| | | | | | sequence. | |
| 700346723H1 | g466687 | 19 | −1 | gb105pln | Arabidopsis thaliana genomic DNA, chromosome 5, P1 clone: MAC12, complete | |
| | | | | | sequence. | |
| 700345538H1 | g218099 | 25 | −10 | gb105pln | Rice mRNA for ribosomal protein S12 (320 gene), partial sequence. | |
| 700345772H1 | g699510 | 13 | −4 | gb105eukp | M(1)1B; minute(1)1B protein; minute(1)1B | |
| 700347383H1 | g992962 | 21 | −2 | gb105eukp | thioredoxin | |
| 700345824H1 | g599624 | 46 | −34 | gb105pln | A. thaliana mRNA for aconitase (ZAPII). | |
| 700345262H1 | g168460 | 63 | −51 | gb105pln | Zea mays cyclophilin (CyP) mRNA, complete cds. | |
| 700380535H1 | g310932 | 45 | −43 | gb105pln | Nicotiana tabacum ribosomal protein L17 mRNA, complete cds. | |
| 700379760H1 | g2494128 | 14 | −10 | gb105eukp | T1G11.12 | |
| 700346073H1 | g1928865 | 45 | −29 | gb105pln | Triticum aestivum root abundant protein mRNA, complete cds. | |
| 700344038H1 | g218133 | 19 | 6 | gb105pln | Rice mRNA for Ribosomal protein L32. | |
| 700346623H1 | g556685 | 35 | −14 | gb105pln | Z. mays mRNA for ADP-ribosylation factor. | |
| 700346703H1 | g514319 | 24 | −8 | gb105pln | Arabidopsis thaliana RNA polymerase II third largest subunit gene, | |
| | | | | | complete cds. | |
| 700344138H1 | g2193870 | 11 | 7 | gb105allp | reverse transcriptase | |
| 700346243H1 | g31415 | 25 | −9 | gb105allp | fibulin-1 A | |
| 700344036H1 | g514945 | 100 | −34 | gb105pln | Zea mays sucrose synthase (Sus1) mRNA, complete cds. | |
| 700343654H1 | g142464 | 12 | 3 | gb105allp | ribosomal protein L17 | |
| 700379937H1 | g248338 | 98 | −85 | gb105pln | polyubiquitin [maize, Genomic, 3439 nt]. | |
| 700379658H1 | g2739218 | 52 | −37 | gb105pln | Hordeum vulgare S28 ribosomal protein. | |
| 700347254H1 | g170746 | 50 | −50 | gb105pln | Wheat histone H4 TH091 gene, complete cds. | |
| 700342844H1 | g18140 | 14 | 13 | gb105pln | C. rubrum mRNA for light-induced 34 kD protein. | |
| 700342237H1 | g1906600 | 10 | −4 | gb105eukp | mepB; MepB | |
| 700379902H1 | g1016347 | 23 | 2 | gb105eukp | DCUP; uroporphyrinogen decarboxylase; EC 4.1.1.37 | |
| 700344653H1 | g1325967 | 32 | 11 | gb105pln | T. aestivum histone H2A gene (clone TH274). | |
| 700342736H1 | g2564336 | 24 | −5 | gb105pln | Brassica campestris mRNA for Tat binding protein 1, complete cds. | |
| 700346641H1 | g2276367 | 14 | 0 | gb105allp | 60s ribosomal protein | |
| 700281848H2 | g218130 | 38 | −11 | gb105pln | Rice mRNA for Ribosomal protein S15. | |
| 700346872H1 | g1184107 | 15 | 3 | gb105allp | DNA replication initiator protein | |
| 700345668H1 | g1045254 | 20 | −7 | gb105eukp | RPL30B | |
| 700342651H1 | g1402874 | 27 | −0 | gb105pln | A. thaliana 81 kb genomic sequence. | |
| 700342216H1 | g1256258 | 21 | 1 | gb105pln | Spinacia oleracea voltage-dependent anion channel protein (SVDAC1) mRNA, | |
| | | | | | complete cds. | |
| 700380432H1 | g2264305 | 12 | 15 | gb105pln | Arabidopsis thaliana genomic DNA, chromosome 5, P1 clone: MBK23, complete | |
| | | | | | sequence. | |
| 700344133H1 | g22314 | 82 | −85 | gb105pln | Maize mRNA for GSH gluthathione S-transferase I (GST; EC 2.5.1.18). | |
| 700343440H1 | g2245113 | 40 | −18 | gb105eukp | glycerol-3-phosphate permease homolog | |
| 700345959H1 | g1675393 | 24 | −24 | gb105pln | Oryza sativa class III ADH enzyme (AdhIII) gene, complete cds. | |
| 700342529H1 | g577824 | 40 | −41 | gb105pln | Z. mays gene for H2B histone (gH2B3). | |
| 700345562H1 | g493590 | 61 | −55 | gb105pln | Hordeum vulgare disulfide isomerase (PDI) mRNA, 3′ end. | |
| 700346211H1 | g453692 | 11 | 0 | gb105allp | AT hook 1 from AA 98-106, AT hook 2 from AA 129-137, AT hook 3 from AA | |
| | | | | | 154-162, AT hook 4 from AA 192-200 | |
| 700345460H1 | g545211 | 41 | −18 | gb105eukp | nda4+ | |
| 700281828H2 | g248336 | 37 | −64 | gb105pln | polyubiquitin [maize, Genomic, 3841 nt]. | |
| 700343485H1 | g1181672 | 62 | −35 | gb105pln | Sorghum bicolor heat shock protein 70n cognate (hsc70) mRNA, partial cds. | |
| 700380064H1 | g387908 | 38 | −22 | gb105pln | Brassica rapa S-phase-specific (BIS289) mRNA, complete cds. | |
| 700343320H1 | g1230676 | 15 | 12 | gb105pln | Yeast (Saccharomyces cerevisiae) chromosome XVI cosmid 9513. | |
| 700281846H2 | g2414629 | 12 | 4 | gb105eukp | SPAC2C4.18; hypothetical protein | |
| 700347246H1 | g286001 | 9 | 3 | gb105allp | KIAA0005 | |
| 700346494H1 | g2342735 | 24 | −13 | gb105eukp | T14G11.28 | |
| 700344228H1 | g1325967 | 13 | 4 | gb105pln | T. aestivum histone H2A gene (clone TH274). | |
| 700344052H1 | g486248 | 22 | −9 | gb105pln | Yeast (Saccharomyces cerevisiae) chromosome XI reading frame ORF YKL145w. | |
| 700343982H1 | g1326067 | 33 | 0 | gb105allp | MaxiK potassium channel beta subunit | |
| 700344916H1 | g310932 | 35 | −26 | gb105pln | Nicotiana tabacum ribosomal protein L17 mRNA, complete cds. | |
| 700343122H1 | g777757 | 63 | −33 | gb105pln | Saccharum hybrid (clone SCUBI561) polyubiquitin mRNA, complete cds. | |
| 700379629H1 | g416256 | 15 | 12 | gb105pln | Rice mRNA for H+-ATPase, partial sequence. | |
| 700344637H1 | g2190276 | 26 | −10 | gb105eukp | alpha-glucosidase; EC 3.2.1.20 | |
| 700347336H1 | g2149018 | 19 | −1 | gb105pln | Oryza sativa putative ADP-glucose pyrophosphorylase subunit SH2 and | |
| | | | | | putative NADPH-dependent reductase A1 genes, complete cds. | |
| 700345527H1 | g21794 | 66 | −54 | gb105pln | Wheat histone H4 gene. | |
| 700347353H1 | g2511530 | 76 | −72 | gb105pln | Eleusine indica alpha tubulin 1 (TUA1) mRNA, complete cds. | |
| 700346111H1 | g2511573 | 16 | −23 | gb105pln | Arabidopsis thaliana mRNA for proteasome subunit prc3. | |
| 700342384H1 | g1685071 | 20 | 0 | gb105allp | ribosomal protein S5 | |
| 700342465H1 | g466439 | 26 | −4 | gb105allp | 40 kDa ribosomal protein | |
| 700379754H1 | g17484 | 11 | 8 | gb105allp | Tumor protein | |
| 700343901H1 | g2662313 | 68 | −65 | gb105pln | Hordeum vulgare mRNA for bpw3, complete cds. | |
| 700345476H1 | g2276367 | 25 | −10 | gb105eukp | SPBC30D10.18c; 60s ribosomal protein | |
| 700343165H1 | g1786197 | 13 | 2 | gb105allp | DnaJ protein | |
| 700345087H1 | g1872162 | 40 | −29 | gb105pln | Arabidopsis thaliana DnaJ homolog (atj) mRNA, complete cds. | |
| 700344865H1 | g2511530 | 42 | −33 | gb105pln | Eleusine indica alpha tubulin 1 (TUA1) mRNA, complete cds. | |
| 700346017H1 | g2582057 | 65 | 2 | gb105allp | NADH dehydrogenase subunit 2 | |
| 700345490H1 | g1419459 | 18 | −14 | gb105eukp | mei-1; T01G9.5 | |
| 700343719H1 | g595415 | 30 | −12 | gb105eukp | sec27; betaprime COP | |
| 700342733H1 | g547390 | 33 | −26 | gb105pln | Arabidopsis thaliana TRAP mRNA, partial cds. | |
| 700342459H1 | g644492 | 74 | −66 | gb105pln | Corn elongation factor 1alpha gene, complete cds. | |
| 700344147H1 | g4899 | 21 | −7 | gb105pln | Yeast (Schizosaccharomyces pombe) act1 gene for actin. | |
| 700342296H1 | g36049 | 24 | −1 | gb105allp | precursor | |
| 700347076H1 | g529357 | 22 | −14 | gb105eukp | T12A2.2 | |
| 700344374H1 | g168423 | 56 | −55 | gb105pln | Zea mays polypeptide chain-binding protein mRNA, 3′ end. | |
| 700343176H1 | g2760325 | 9 | 6 | gb105eukp | F1N21.10 | |
| 700345875H1 | g1435156 | 50 | −35 | gb105pln | L. esculentum mRNA for histone H3 variant H3.3. | |
| 700347009H1 | g166680 | 15 | 5 | gb105allp | protein kinase | |
| 700344093H1 | g310322 | 26 | −37 | gb105pln | Oryza sativa triosephosphate isomerase (Rictipi2) gene, exons 1-9. | |
| 700345454H1 | g1405747 | 10 | 5 | gb105allp | PR264/SC35 | |
| 700344805H1 | g2570506 | 47 | −35 | gb105pln | Oryza sativa ribosomal protein mRNA, complete cds. | |
| 700342238H1 | g2264306 | 13 | 16 | gb105pln | Arabidopsis thaliana genomic DNA, chromosome 5, P1 clone: MBK 5, complete | |
| | | | | | sequence. | |
| 700346388H1 | g168508 | 16 | 11 | tgb105pln | Maize oleosin KD18 (KD18; L2) gene, complete cds. | |
| 700346290H1 | g170455 | 47 | −31 | gb105pln | Tomato heat shock cognate protein 80 gene, 3′ end. | |
| 700345178H1 | g1617015 | 19 | 8 | gb105eukp | ribonucleotide reductase RNR1 like protein | |
| 700345677H1 | g2827001 | 56 | −42 | gb105pln | Triticum aestivum 70 kDa heat shock protein (TaHSP70d) mRNA, complete cds. | |
| 700344859H1 | g1806617 | 12 | 6 | gb10allp | spindle assembly checkpoint component | |
| 700343477H1 | g2352928 | 33 | −10 | gb105pln | Petroselinum crispum plastidic chorismate mutase 1 (pCM1) mRNA, nuclear gene | |
| | | | | | encoding plastid protein, partial cds. | |
| 700344687H1 | g1431629 | 26 | −16 | gb105eukp | pectinacetylesterase precursor | |
| 700343829H1 | g22328 | 55 | −39 | gb105pln | Maize mRNA for a high mobility group protein. | |
| 700346012H1 | g498761 | 26 | −3 | gb105eukp | YBR0320 | |
| 700345807H1 | g2230869 | 91 | 5 | gb105allp | ferritin L-chain | |
| 700343150H1 | g1045057 | 6 | 7 | gb105allp | ch-TOG | |
| 700347130H1 | g2293568 | 43 | 2 | gb105eukp | HvB12D homolog | |
| 700344204H1 | g2645162 | 18 | −1 | gb105pln | Oryza sativa mRNA, similar to initiation factor eIF-4c. | |
| 700344001H1 | g21800 | 42 | 11 | gb105pln | T. aestivum L mRNA for histone H2B. | |
| 700346845H1 | g2564003 | 22 | 0 | gb105allp | proteasome p45/SUG | |
| 700342427H1 | g303856 | 46 | −27 | gb105pln | Rice mRNA for ubiquitin protein fused to a ribosomal protein, complete cds. | |
| 700380712H1 | g21800 | 40 | −39 | gb105pln | T. aestivum L mRNA for histone H2B. | |
| 700380518H1 | g1906825 | 27 | −13 | gb105pln | A. thaliana hsp81.2 gene. | |
| 700347132H1 | g644491 | 77 | −16 | gb105pln | Corn mRNA for elongation factor 1A. | |
| 700345410H1 | g2505874 | 50 | −26 | gb105eukp | putative kinase | |
| 700347257H1 | g303858 | 49 | −41 | gb105pln | Rice mRNA for brain specific protein (S94 gene), complete cds. | |
| 700347040H1 | g2317855 | 15 | 3 | gb105eukp | MMS2; provides resistance to DNA damaging agents; Mms2p | |
| 700344195H1 | g1663702 | 25 | 7 | gb105allp | KIAA0243 | |
| 700343925H1 | g6634 | 16 | 5 | gb105eukp | B0464.1 | |
| 700343863H1 | g1122316 | 41 | −2 | gb105pln | P. glaucum mRNA for heat shock protein, HSP 17.9. | |
| 700342583H1 | g984261 | 89 | −74 | gb105pln | Z. mays mRNA for aspargine synthetase. | |
| 700342858H1 | g2429087 | 9 | −3 | gb105eukp | LoxC; lipoxygenase 2; EC 1.13.11.12 | |
| 700345556H1 | g1160445 | 6 | 6 | gb105allp | ribosomal protein S7 | |
| 700343778H1 | g170373 | 24 | −23 | gb105pln | Tomato 5-enolpyruvylshikimate-3-phosphate synthase mRNA, complete cds. | |
| 700346013H1 | g497629 | 15 | 4 | gb105allp | ERD1 protein | |
| 700343942H1 | g515376 | 67 | −60 | gb105pln | L. temulentum mRNA for histone H4. | |
| 700343142H1 | g2832611 | 27 | −3 | gb105pln | Arabidopsis thaliana DNA chromosome 4, BAC clone F13C5 (ESSAII project). | |
| 700346426H1 | g1134968 | 10 | 6 | gb105eukp | protein disulphide isomerase PDI; EC 5.3.4.1 | |
| 700343158H1 | g2351062 | 21 | 13 | gb105pln | Arabidopsis thaliana genomic DNA, chromosome 5, P1 clone: MAH20. | |
| 700343495H1 | g1519248 | 22 | −4 | gb105pln | Oryza sativa GF14-b protein mRNA, complete cds. | |
| 700343131H1 | g1561732 | 9 | 6 | gb105eukp | Reg-2; Dreg-2 protein | |
| 700343804H1 | g556872 | 9 | 1 | gb105eukp | sen3 | |
| 700346056H1 | g1707641 | 28 | −13 | gb105pln | O. sativa mRNA for leucine rich repeat receptor-like kinase. | |
| 700343928H1 | g1469218 | 45 | −36 | gb105pln | B. oleracea mRNA (unknown). | |
| 700343684H1 | g1122778 | 9 | −1 | gb105eukp | C10C5.5 | |
| 700346979H1 | g409072 | 18 | 2 | gb105allp | HBp15/L22 | |
| 700342862H1 | g168460 | 77 | −13 | gb105pln | Zea mays cyclophilin (CyP) mRNA, complete cds. | |
| 700345459H1 | g2760844 | 14 | 8 | gb105eukp | F18A8.14 | |
| 700346427H1 | g2829889 | 20 | −7 | gb105eukp | F3I6.29 | |
| 700343516H1 | g780371 | 51 | −56 | gb105pln | Oryza sativa enolase mRNA, complete cds. | |
| 700347326H1 | g780371 | 54 | −43 | gb105pln | Oryza sativa enolase mRNA, complete cds. | |
| 700346944H1 | g1835684 | 30 | −11 | gb105eukp | PIP2; Pip2p | |
| 700344961H1 | g2244785 | 31 | −8 | gb105eukp | hypothetical protein | |
| 700345333H1 | g1928865 | 38 | −14 | gb105pln | Triticum aestivum root abundant protein mRNA, complete cds. | |
| 700343543H1 | g2194125 | 34 | −19 | gb105eukp | F20P5.12 | |
| 700281860H2 | g886737 | 57 | −10 | gb105pln | Z. mays histone H3 gene. | |
| 700343430H1 | g899609 | 68 | −78 | gb105pln | Zea mays acidic ribosomal protein P2 (RPA-2A1) mRNA, complete cds. | |
| 700345668H1 | g1644321 | 28 | −11 | gb105eukp | SPAC6G9.09c; hypothetical protein | |
| 700344095H1 | g1041767 | 50 | −45 | gb105pln | Acer pseudoplatanus V-ATPase 66 kDa subunit mRNA, partial cds. | |
| 700344276H1 | g565064 | 27 | −7 | gb105pln | Arabidopsis thaliana pyruvate dehydrogenase E1 beta subunit mRNA, complete cds. | |
| 700343804H1 | g763271 | 9 | 1 | gb105eukp | SEN3; Sen3p | |
| 700345087H1 | g454913 | 41 | −31 | gb105pln | A. porrum LDJ2 mRNA. | |
| 700342537H1 | g2662346 | 77 | −65 | gb105pln | Oryza sativa mRNA for EF-1 alpha, complete cds. | |
| 700345588H1 | g22681 | 43 | −33 | gb105pln | L. usitatissimum mRNA for Stearoyl-(acyl-carrier-protein)desaturase. | |
| 700379660H1 | g798817 | 26 | −16 | gb105pln | A. thaliana mRNA for ribosomal protein L2. | |
| 700343012H1 | g21800 | 41 | −41 | gb105pln | T. aestivum L mRNA for histone H2B. | |
| 700346413H1 | g836939 | 17 | −14 | gb105pln | Arabidopsis thaliana calcium-dependent protein kinase (CDPK6) mRNA, complete cds. | |
| 700343836H1 | g289658 | 54 | −29 | gb105eukp | C50C3.6 protein | |
| 700342738H1 | g2239150 | 37 | −49 | gb105pln | N. tabacum mRNA for CHLD magnesium chelatase subunit. | |
| 700342393H1 | g30377 | 43 | −31 | gb105allp | cytokeratin 13 | |
| 700342321H1 | g1256265 | 25 | −11 | gb105allp | protein kinase C inhibitor | |
| 700343350H1 | g531055 | 38 | −22 | gb105pln | Wheat mRNA for protein H2B-6, complete cds. | |
| 700343020H1 | g1575548 | 11 | −4 | gb105allp | methoxy mycolic acid synthase 3 | |
| 700344951H1 | g1519248 | 53 | −48 | gb105pln | Oryza sativa GF14-b protein mRNA, complete cds. | |
| 700281817H2 | g 602761 | 10 | 3 | gb105allp | RecA protein | |
| 700380417H1 | g600115 | 11 | 7 | gb105pln | Z. mays apx gene encoding cytosolic ascorbate peroxidase. | |
| 700380043H1 | g2244901 | 12 | 13 | gb105pln | Arabidopsis thaliana DNA chromosome 4, ESSA I contig fragment No. 4. | |
| 700346045H1 | g167116 | 24 | −21 | gb105pln | B. napus plastid 60- kDa chaperonin-60 beta-polypeptide (cpn-60 beta) mRNA, | |
| | | | | | partial cds. | |
| 700379982H1 | g1395177 | 19 | 6 | gb105allp | proteasomal ATPase (rat SUG1) | |
| 700379957H1 | g940843 | 14 | −1 | gb105eukp | orf 06116 | |
| 700343253H1 | g1546918 | 70 | −80 | gb105pln | Z. mays mRNA for translation initiation factor 5A. | |
| 700347254H1 | g21794 | 47 | −48 | gb105pln | Wheat histone H4 gene. | |
| 700345454H1 | g63752 | 10 | 6 | gb105allp | PR 264 | |
| 700343489H1 | g168602 | 74 | −45 | gb105pln | Zea mays regulatory protein GF14-12 mRNA, complete cds. | |
| 700347121H1 | g469147 | 54 | −52 | gb105pln | H. vulgare mRNA for alanine aminotransferase. | |
| 700344805H1 | g463251 | 28 | −10 | gb105pln | M. sativa (Nagyszenasi) mRNA for ribosomal protein RL5. | |
| 700346057H1 | g22324 | 68 | −57 | gb105pln | Z. mays mRNA for H2B histone (clone cH2B221). | |
| 700281810H2 | g2529427 | 24 | 0 | gb105eukp | rpc10+; rpc10; EC 2.7.7.6 | |
| 700346469H1 | g22324 | 51 | −44 | gb105pln | Z. mays mRNA for H2B histone (clone cH2B221). | |
| 700343233H1 | g2388937 | 23 | −8 | gb105eukp | SPAC23H4.18c; hypothetical protein | |
| 700379926H1 | g218000 | 32 | −33 | gb105pln | Potato mRNA for UDP-glucose pyrophosphorylase (EC 2.7.7.9). | |
| 700343651H1 | g1498052 | 60 | −56 | gb105pln | Zea mays ribosomal protein S8 mRNA, complete cds. | |
| 700343740H1 | g1769895 | 11 | −1 | gb105eukp | CaLB protein | |
| 700346342H1 | g489431 | 13 | 4 | gb105allp | PP4X | |
| 700346421H1 | g473604 | 24 | −14 | gb105pln | Zea mays W-22 histone H2B mRNA, complete cds. | |
| 700343233H1 | g2388937 | 23 | −8 | gb105eukp | SPAC23H4.18c; hypothetical protein | |
| 700379926H1 | g218000 | 32 | −33 | gb105pln | Potato mRNA for UDP-glucose pyrophosphorylase (EC 2.7.7.9). | |
| 700343651H1 | g1498052 | 60 | −56 | gb105pln | Zea mays ribosomal protein S8 mRNA, complete cds. | |
| 700343740H1 | g1769895 | 11 | −1 | gb105eukp | CaLB protein | |
| 700346342H1 | g489431 | 13 | 4 | gb105allp | PP4X | |
| 700346421H1 | g473604 | 24 | −14 | gb105pln | Zea mays W-22 histone H2B mRNA, complete cds. | |
| 700344366H1 | g557681 | 59 | −48 | gb105pln | Z. mays mRNA for ribosomal protein S28. | |
| 700346364H1 | g217973 | 33 | −56 | gb105pln | Zea mays gene for triosephosphate isomerase, complete cds. | |
| 700343550H1 | g886450 | 14 | 5 | gb105allp | F54C9.5 | |
| 700343649H1 | g168490 | 59 | −54 | gb105pln | Maize glutathione S-transferase (GST-I) mRNA, complete cds. | |
| 700342582H1 | g2326453 | 15 | 1 | gb105eukp | GPxha-1; glutathione peroxidase; EC 1.11.1.9 | |
| 700342545H1 | g2351689 | 18 | −5 | gb105eukp | ap51-3; cytoadherence to epithelial cell surface; adhesin protein AP51-3 | |
| 700346785H1 | g1542940 | 27 | −27 | gb105pln | R. sativus L. (Saxa knacker) AACT mRNA. | |
| 700343562H1 | g683683 | 18 | −6 | gb105eukp | NOP1; nucleolar protein NOP1 (J05230); D2870 | |
| 700379854H1 | g2264312 | 17 | 5 | gb105pln | Arabidopsis thaliana genomic DNA, chromosome 5, P1 clone: MOK16, | |
| | | | | | complete sequence. | |
| 700344330H1 | g1255448 | 25 | −7 | gb105eukp | ATMEKK1; mitogen-activated protein kinase | |
| 700343363H1 | g2791948 | 21 | −8 | gb105eukp | rp113a; ribosomal protein L13a | |
| 700344794H1 | g397395 | 33 | −25 | gb105pln | Z. mays MNB1b mRNA for DNA-binding protein. | |
| 700380220H1 | g456671 | 40 | −30 | gb105pln | T. aestivum VDAC 1 mRNA. | |
| 700343584H1 | g1181332 | 46 | −44 | gb105pln | Z. mays CRH mRNA. | |
| 700343836H1 | g395157 | 6 | 7 | gb105eukp | PRP8; pre-mRNA splicing factor, U5 snRNP protein; PRP8 | |
| 700343156H1 | g35087 | 8 | 7 | gb105allp | neurite outgrowth-promoting protein | |
| 700343035H1 | g817954 | 13 | 7 | gb105allp | DMR-N9 | |
| 700345251H1 | g1272409 | 23 | −2 | gb105pln | Vicia faba immunophilin precursor (FKBP15) mRNA, complete cds. | |
| 700343562H1 | g1495012 | 20 | −6 | gb105eukp | T01C3.7 | |
| 700379601H1 | g20255 | 42 | −20 | gb105pln | O. sativa gene for heat shock protein 82 HSP82. | |
| 700345452H1 | g1064884 | 34 | −21 | gb105pln | A. thaliana mRNA for phosphoribosyl pyrophosphate synthetase II. | |
| 700347011H1 | g2326453 | 17 | 7 | gb105allp | glutathione peroxidase | |
| 700343686H1 | g2078349 | 30 | −24 | gb105pln | Solanum tuberosum transaldolase (PotTall) mRNA, complete cds. | |
| 700379932H1 | g2827529 | 29 | −6 | gb105eukp | F8F16.160; putative protein | |
| 700345425H1 | g22119 | 84 | −25 | gb105pln | Maize Adh1-F mRNA for alcohol dehydrogenase. | |
| 700344903H1 | g2760323 | 57 | −34 | gb105eukp | F1N21.8 | |
| 700281960H2 | g2398526 | 20 | −4 | gb105pln | Arabidopsis thaliana mRNA for Hap3a transcription factor. | |
| 700380440H1 | g1463028 | 21 | 6 | gb105allp | voltage dependent anion channel 3 | |
| 700343928H1 | g2584827 | 28 | −7 | gb105pln | Sequence of BAC F12F1 from Arabidopsis thaliana chromosome 1, complete | |
| | | | | | sequence. | |
| 70034388H1 | g1277236 | 13 | −5 | gb105eukp | SEC13; Sec13p | |
| 700343842H1 | g189306 | 7 | 6 | gb105allp | nucleolin | |
| 700344231H1 | g168502 | 47 | −38 | gb105pln | Maize (Zea mays) histone H4 gene (H4C7), complete cds. | |
| 700347240H1 | g310932 | 43 | −29 | gb105pln | Nicotiana tabacum ribosomal protein L17 mRNA, complete cds. | |
| 700345257H1 | g297172 | 13 | 6 | gb105allp | ribosomal protein S7 | |
| 700342308H1 | g1498385 | 82 | −73 | gb105pln | Zea mays actin (Maz87) gene, partial cds. | |
| 700344746H1 | g1244565 | 23 | −30 | gb105pln | Triticum aestivum acetyl-CoA carboxylase gene, exons 1-30, complete cds. | |
| 700380443H1 | g435648 | 34 | −43 | gb105pln | Rice mRNA for gamma-Tip, complete cds. | |
| 700346005H1 | g57710 | 28 | 3 | gb105allp | ribosomal phosphoprotein P1 (AA 1-114) | |
| 700342288H1 | g2463332 | 17 | −6 | gb105pln | Oryza sativa mRNA for M79 protein. | |
| 700346875H1 | g2098816 | 17 | 8 | gb105pln | Arabidopsis thaliana BAC F19G10, complete sequence. | |
| 700380479H1 | g311297 | 14 | 8 | gb105allp | ribosomal protein S24 | |
| 700346069H1 | g292443 | 40 | −9 | gb105allp | ribosomal protein S20 | |
| 700345334H1 | g968901 | 23 | 7 | gb105pln | Rice mRNA for ribosomal protein S8, complete cds. | |
| 700345103H1 | g485952 | 39 | −40 | gb105pln | O. sativa mRNA for glutaredoxin | |
| 700379829H1 | g303852 | 64 | −61 | gb105pln | Rice mRNA for ribosomal protein L3, complete cds. | |
| 700379665H1 | g600115 | 30 | −12 | gb105pln | Z. mays apx gene encoding cytosolic ascorbate peroxidase. | |
| 700346902H1 | g899609 | 60 | −44 | gb105pln | Zea mays acidic ribosomal protein P2 (RPA-2A1) mRNA, complete cds. | |
| 700344648H1 | g506470 | 39 | −28 | gb105pln | N. tabacum mRNA pNLA-35. | |
| 700343016H1 | g303499 | 19 | −3 | gb105pln | Arabidopsis thaliana Atpk7 gene encoding serine/threonine protein kinase. | |
| 700379974H1 | g927571 | 93 | −78 | gb105pln | Z. mays mRNA for calreticulin precursor. | |
| 700379985H1 | g218160 | 35 | −17 | gb105pln | Oryza sativa mRNA for elongation factor 1 beta′. | |
| 700346636H1 | g577824 | 24 | −25 | gb105pln | Z. mays gene for H2B histone (gH2B3). | |
| 700345741H1 | g32101 | 71 | −20 | gb105allp | hinge protein precursor (AA-13 to 78) | |
| 700343295H1 | g2842492 | 15 | −1 | gb105eukp | F21O9.180; protein kinase Xa21 homolog | |
| 700346621H1 | g2627180 | 28 | −29 | gb105pln | Pisum sativum mRNA for cycloartenol synthase, complete cds. | |
| 700345281H1 | g168460 | 68 | −61 | gb105pln | Zea mays cyclophilin (CyP) mRNA, complete cds. | |
| 700343920H1 | g625108 | 48 | −8 | gb105eukp | YLR388W; Ylr388wp | |
| 700346645H1 | g623207 | 12 | −1 | gb105eukp | mrp-3; ribosomal protein | |
| 700344742H1 | g293888 | 75 | −69 | gb105pln | Zea mays, glyceraldehyde-3-phosphate dehydrogenase mRNA, 3′ end (clone GAPC2). | |
| 700344702H1 | g416146 | 79 | “67 | gb105pln | Zea mays beta-6 tubulin (tub6) gene and mRNA, complete cds. | |
| 700347351H1 | g397632 | 37 | −33 | gb105pln | T. aestivum translation initiation factor 4A. | |
| 700344742H1 | g22302 | 47 | −76 | gb105pln | Maize Gpcl gene for glyceraldehyde-3-phosphate dehydrogenase (GADPH) subunit C. | |
| 700347171H1 | g1420235 | 16 | 5 | gb105allp | ORF YOR077w | |
| 700345696H1 | g717191 | 64 | 2 | gb105allp | RNA polymerase II subunit | |
| 700344840H1 | g1899026 | 24 | −35 | gb105pln | Zea mays superoxide dismutase 4A (sod4A) gene, complete cds. | |
| 700347034H1 | g1568479 | 12 | 15 | gb105pln | B. vulgaris partial cdc2-related protein kinase gene. | |
| 700343559H1 | g440952 | 23 | −10 | gb105pln | cytochrome cl [Solanum tuberosum = potatoes, Desiree, monohaploid line AM 80/5793, | |
| | | | | | Genomic, 6007 nt]. | |
| 700379985H1 | g218340 | 37 | −19 | gb105pln | Triticum aestivum mRNA for elongation factor 1 beta′. | |
| 700380481H1 | g2276360 | 10 | 4 | gb105allp | Gpilp | |
| 700347327H1 | g2702261 | 12 | 13 | gb105pln | Arabidopsis thaliana chromosome II BAC T21L14 genomic sequence, complete | |
| | | | | | sequence. | |
| 700344893H1 | g2331133 | 6 | 6 | gb105allp | glycine-rich protein | |
| 700281841H2 | g2511530 | 67 | −70 | gb105pln | Eleusine indica alpha tubulin 1 (TUA1) mRNA, complete cds. | |
| 700346250H1 | g683475 | 19 | −22 | gb105pln | H. vulgare mRNA for NADPH-protochlorophyllide oxidoreducatase. | |
| 700345508H1 | g1208445 | 18 | 15 | gb105pln | Rice (YK426) mRNA, complete cds. | |
| 700345988H1 | g1632830 | 31 | −5 | gb105pln | R. communis mRNA (unknown). | |
| 700344542H1 | g551625 | 7 | 6 | gb105allp | protease II | |
| 700346067H1 | g349403 | 15 | 17 | gb105pln | Brassica campestris (clone lambda CGN1-2) napin gene, complete cds. | |
| 700343248H1 | g722325 | 26 | 2 | gb105pln | Zea mays clone Zm-Rab2-A GTP binding protein (rab2) mRNA, complete cds. | |
| 700346664H1 | g20255 | 70 | −57 | gb105pln | O. sativa gene for heat shock protein 82 HSP82. | |
| 700344430H1 | g577824 | 26 | −27 | gb105pln | Z. mays gene for H2B histone (gH2B3). | |
| 700342230H1 | g758339 | 34 | −16 | gb105pln | S. tuberosum mRNA for 76 kDa mitochondrial complex I subunit. | |
| 700380552H1 | g182514 | 26 | 1 | gb105allp | ferritin light chain | |
| 700343045H1 | g1850559 | 12 | 4 | gb105allp | macrophage migration inhibitory factor | |
| 700342420H1 | g21796 | 34 | −44 | gb105pln | Wheat histone H3 gene. | |
| 700380280H1 | g536073 | 12 | −8 | gb105eukp | ORF YBL049w | |
| 700346634H1 | g310932 | 35 | −24 | gb105pln | Nicotiana tabacum ribosomal protein L17 mRNA, complete cds. | |
| 700344925H1 | g450292 | 91 | −75 | gb105pln | Zea mays alpha-tubulin mRNA, complete cds. | |
| 700343339H1 | g1177435 | 18 | 4 | gb105allp | 14.5 kDa translational inhibitor protein, p14.5 | |
| 700342208H1 | g790507 | 17 | −14 | gb105pln | Z. mays mRNA for 60S acidic ribosomal protein. | |
| 700346380H1 | g2182266 | 21 | 5 | gb105pln | Hordeum vulgare lipoxygenase (LoxB) mRNA, complete cds. | |
| 700343542H1 | g1652223 | 6 | 8 | gb105allp | ABC1-like | |
| 700343834H1 | g1100222 | 34 | −18 | gb105pln | Pinus sylvestris chloroplast AND+-dependent glyceraldehyde-3-phosphate dehydrogenase | |
| | | | | | mRNA, complete cds. | |
| 700344184H1 | g606969 | 31 | −15 | gb105pln | Arabidopsis thaliana cytoplasmic ribosomal protein L18 mRNA, complete cds. | |
| 700342724H1 | g2104681 | 13 | 4 | gb105allp | transcription factor | |
| 700346988H1 | g171596 | 15 | 0 | gb105eukp | D-fructose-6-phosphate amidotransferase (EC 2.6.1.16) | |
| 700344273H1 | g992962 | 13 | −5 | gb105eukp | thioredoxin | |
| 700343165H1 | g216441 | 13 | 2 | gb105allp | dnaJ protein | |
| 700346475H1 | g170107 | 15 | 8 | gb105eukp | CPN10; chaperonin 10 | |
| 700344396H1 | g415316 | 30 | −6 | gb105pln | Rice mRNA for acidic ribosomal protein PO, complete cds. | |
| 700343919H1 | g577824 | 69 | −81 | gb105pln | Z. mays gene for H2B histone (gH2B3). | |
| 700343330H1 | g218180 | 54 | −43 | gb105pln | Rice mRNA for oryzain alpha (EC 3.4.22). | |
| 700380171H1 | g293902 | 11 | −1 | gb105eukp | Zea mI | |
| 700343550H1 | g173232 | 13 | 5 | gb105eukp | YL3; 5S ribosomal RNA binding-protein | |
| 700346117H1 | g2814799 | 27 | −14 | gb105eukp | ZK858.6 | |
| 700345577H1 | g407800 | 24 | 6 | gb105pln | G. hirsutum mRNA for ribosomal protein 41, large subunit (RL41). | |
| 700346655H1 | g1703576 | 7 | 7 | gb105eukp | C43E11.1 | |
| 700346080H1 | g8394 | 10 | 0 | gb105eukp | pum | |
| 700344205H1 | g218088 | 37 | −67 | gb105pln | Rice mRNA for ribosomal protein 117 (249 gene), partial sequence. | |
| 700343645H1 | g22651 | 39 | −26 | gb105pln | H. vulgare mRNA for GRP94 homologue. | |
| 700380194H1 | g829024 | 25 | −6 | gb105allp | ATPase | |
| 700345346H1 | g189979 | 6 | 8 | gb105allp | protein kinase C alpha-polypeptide | |
| 700343918H1 | g493590 | 59 | −53 | gb105pln | Hordeum vulgare disulfide isomerase (PDI) mRNA, 3′ end. | |
| 700342440H1 | g2606079 | 25 | −27 | gb105eukp | ribosomal L15 (YL10) protein homologue | |
| 700342858H1 | g532572 | 11 | −1 | gb105eukp | LoxA; lipoxygenase 1; EC 1.13.11.12; linoleate:oxygen oxioreductase, lipoxidase, | |
| | | | | | carotene oxidase | |
| 700345913H1 | g4392 | 14 | −1 | gb105eukp | RPL37A; ribosomal protein L37a | |
| 700344274H1 | g1652418 | 9 | 5 | gb105allp | 30S ribosomal protein S17 | |
| 700342350H1 | g1429215 | 12 | 6 | gb105eukp | peroxidase ATP6a; EC 1.11.1.7 | |
| 700347166H1 | g1107484 | 20 | 1 | gb105pln | A. thaliana mRNA for 40S ribosomal protein S15. | |
| 700346101H1 | g551482 | 12 | 7 | gb105pln | Zea mays ABA- and ripening-inducible-like protein mRNA, complete cds. | |
| 700344977H1 | g1321740 | 17 | 7 | gb105eukp | B0035.3 | |
| 700343587H1 | g21234 | 21 | −8 | gb105eukp | AHRI; acetohydroxy acid reductoisomerase; EC 1.1.1.86 | |
| 700344527H1 | g172766 | 15 | 0 | gb105eukp | heat shock protein STGI1 | |
| 700342367H1 | g902583 | 35 | −47 | gb105pln | Zea mays clone MubG1 ubiquitin gene, complete cds. | |
| 700379908H1 | g642243 | 19 | −2 | gb105pln | H. vulgare cdr29 gene. | |
| 700345771H1 | g2444419 | 35 | −21 | gb105pln | Glycine max ribosome-associated protein p40 mRNA, complete cds. | |
| 700379782H1 | g2739218 | 39 | −10 | gb105pln | Hordeum vulgare S28 ribosomal protein. | |
| 700346629H1 | g218180 | 21 | −13 | gb105pln | Rice mRNA for oryzain alpha (EC 3.4.22). | |
| 700346571H1 | g1292907 | 25 | −11 | gb105pln | Arabidopsis thaliana GTP binding protein (ARAC1) mRNA, complete cds. | |
| 700343743H1 | g949877 | 34 | −38 | gb105pln | H. vulgare mRNA for elongation factor 1-alpha. | |
| 700281813H2 | g2244971 | 21 | −3 | gb105eukp | hypothetical protein | |
| 700345113H1 | g391875 | 25 | −3 | gb105eukp | 40S subunit ribosomal protein | |
| 700345622H1 | g1420686 | 17 | −4 | gb104eukp | RPL18B | |
| 700345023H1 | g927567 | 17 | −2 | gb105eukp | actin | |
| 700345016H1 | g1806139 | 33 | 3 | gb105pln | M. sativa mRNA for cdc2 kinase homologue, cdc2MsC. | |
| 700345946H1 | g2440191 | 25 | −12 | gb105eukp | hypothetical protein | |
| 700344907H1 | g21800 | 26 | −30 | gb105pln | T. aestivum L mRNA for histone H2B. | |
| 700342886H1 | g1237101 | 27 | −9 | gb105pln | Arabidopsis thaliana calmodulin-binding protein mRNA, complete cds. | |
| 700281887H2 | g1498052 | 72 | −58 | gb105pln | Zea mays ribosomal protein S8 mRNA, complete cds. | |
| 700347158H1 | g857573 | 39 | −16 | gb105pln | Oryza sativa vacuolar H+-ATPase (vatp-P1) mRNA, complete cds. | |
| 700344612H1 | g602605 | 100 | −86 | gb105pln | Zea mays tandem genes for alpha1-tubulin and alpha2-tubulin. | |
| 700346284H1 | g2668737 | 30 | −32 | gb105pln | Zea mays translation initiation factor 5A (TIF5A) mRNA, complete cds. | |
| 700346255H1 | g1065461 | 10 | 8 | gb105eukp | B0403.4 | |
| 700379664H1 | g170053 | 43 | −35 | gb105pln | Soybean ribosomal protein S11 mRNA, 3′ end. | |
| 700345809H1 | g168500 | 12 | 15 | gb105pln | Maize (Zea mays) histone H4 gene (H4C14), complete cds.) | |
| 700344746H1 | g1469247 | 28 | −29 | gb105pln | H. vulgare mRNA for acetyl-CoA carboxylase. | |
| 700344621H1 | g1244773 | 22 | −7 | gb105eukp | RPL37A; Rp137ap: 60S ribosomal protein L37a | |
| 700345943H1 | g2370557 | 15 | 5 | gb105eukp | SPAC9G1.09; serine threonine protein kinase | |
| 700344522H1 | g1419684 | 35 | −19 | gb105pln | M. sativa mRNA for TCTP-like protein. | |
| 700342427H1 | g347063 | 38 | −21 | gb105pln | Brassica rapa ubiquitin/ribosomal protein mRNA, complete cds. | |
| 700379623H1 | g2245073 | 14 | 4 | gb105pln | Arabidopsis thaliana DNA chromosome 4, ESSA I contig fragment No. 8. | |
| 700343411H1 | g2102693 | 17 | −2 | gb105eukp | Frk2; fructokinase; EC 2.7.1.4 | |
| 700343947H1 | g1089832 | 5 | 5 | gb105allp | T05H10.1 | |
| 700342772H1 | g2599103 | 27 | −15 | gb105pln | Dunaliella saline 60S ribosomal protein (DSRP1) mRNA, complete cds. | |
| 700343649H1 | g168486 | 68 | −67 | gb105pln | Maize glutathione S-transferase gene (GST-I), exon 1. | |
| 700343244H1 | g2224750 | 56 | −41 | gb105pln | Arabidopsis thaliana mRNA for ribosomal protein S6. | |
| 700345594H1 | g293886 | 61 | −73 | gb105pln | Zea mays glyceraldehyde-3-phosphate dehydrogenase mRNA, 3′ end, (clone GAPC3). | |
| 700345568H1 | g2244950 | 20 | 0 | gb105pln | Arabidopsis thaliana DNA chromosome 4, ESSA I contig fragment No. 5. | |
| 700343804H1 | g1055066 | 12 | −1 | gb105eukp | C23G10.4b | |
| 700343380H1 | g1915974 | 23 | −6 | gb105eukp | FK; fructokinase; EC 2.7.1.4 | |
| 700343009H1 | g1154953 | 30 | −41 | gb105pln | T. aestivum histone H2A gene. | |
| 700379794H1 | g577824 | 25 | −6 | gb105pln | Z. mays gene for H2B histone (gH2B3). | |
| 700342372H1 | g168496 | 63 | −67 | gb105pln | Maize (Zea mays) histone H3 gene (H3C4), complete cds. | |
| 700342409H1 | g531057 | 51 | −36 | gb105pln | Wheat mRNA for protein H2B-8, complete cds. | |
| 700345862H1 | g1617270 | 20 | −6 | gb105eukp | acyl-CoA synthetase; EC 6.2.1.3 | |
| 700344005H1 | g2827001 | 49 | −24 | gb105pln | Triticum aestivum 70 kDa heat shock protein (TaHSP70d) mRNA, complete cds. | |
| 700343844H1 | g1185553 | 36 | −76 | gb105pln | Zea mays glyceraldehyde-3-phosphate dehydrogenase (gpc2) gene, complete cds. | |
| 700342637H1 | g453244 | 10 | 6 | gb105allp | catechol O-methyltransferase | |
| 700347328H1 | g22328 | 45 | −20 | gb105pln | Maize mRNA for a high mobility group protein. | |
| 700346116H1 | g299814 | 21 | −17 | gb105pln | tuber-induction gene {3′ region} [Solanum tuberosum = potatoes, cv. Desiree, | |
| | | | | | leaves, mRNA Partial, 950 nt]. | |
| 700342841H1 | g1165205 | 36 | −21 | gb105pln | Arabidopsis thaliana multiubiquitin chain binding protein (MBP1) mRNA, complete cds. | |
| 700344710H1 | g2760173 | 34 | −20 | gb105pln | Arabidopsis thaliana genomic DNA, chromosome 5, P1 clone: MYH19, complete sequence. | |
| 700379728H1 | g436782 | 42 | −39 | gb105pln | Rice mRNA for cyc07, complete cds. | |
| 700345556H1 | g551251 | 6 | 6 | gb105allp | ribosomal protein S7 | |
| 700346687H1 | g1843628 | 35 | −10 | gb105eukp | bsh; SNF5 homolog BSH | |
| 700345690H1 | g35597 | 13 | 3 | gb105allp | PR 264 | |
| 700344201H1 | g1143506 | 9 | 6 | gb105pln | L. luteus mRNA for P0 ribosomal protein. | |
| 700342826H1 | g1498052 | 91 | −75 | gb105pln | Zea mays ribosomal protein S8 mRNA, complete cds. | |
| 700344331H1 | g1418971 | 22 | 2 | gb105pln | H. vulgare mRNA for transcription factor EmBP-1. | |
| 700342584H1 | g2632156 | 25 | −18 | gb105pln | Aspergillus niger mRNA for ribosomal protein L15. | |
| 700346682H1 | g20163 | 40 | −21 | gb105pln | O. sativa Rr15 mRNA for 5S ribosomal RNA. | |
| 700344378H1 | g1185553 | 37 | −72 | gb105pln | Zea mays glyceraldehyde-3-phosphate dehydrogenase (gpc2) gene, complete cds. | |
| 700343079H1 | g1945609 | 9 | 7 | gb105allp | 26S proteasome subunit p44.5 | |
| 700346835H1 | g1628588 | 16 | −0 | gb105pln | Yeast (Candida albicans) glyceraldehyde-3-phosphate dehydrogenase (CaGAP) gene, | |
| | | | | | complete cds. | |
| 700346319H1 | g22469 | 66 | −64 | gb105pln | Maize mRNA for cytoplasmic ribosomal protein S11. | |
| 700380780H1 | g1495250 | 20 | −4 | gb105pln | A. thaliana mRNA for heat-shock protein. | |
| 700380547H1 | g1546918 | 54 | −64 | gb105pln | Z. mays mRNA for translation initiation factor 5A. | |
| 700380179H1 | g1129084 | 25 | −7 | gb105pln | Wheat mRNA for protein H2A, complete cds, clone wcH2A-3. | |
| 700346829H1 | g1718497 | 48 | −21 | gb105eukp | ARP3; actin-related protein 3 | |
| 700347001H1 | g1906825 | 55 | −46 | gb105pln | A. thaliana hsp81.2 gene. | |
| 700346325H1 | g2654121 | 36 | −25 | gb105pln | Arabidopsis thaliana ribosomal protein L23a (AtrpL23a) mRNA, complete cds. | |
| 700343515H1 | g2501849 | 43 | −38 | gb105pln | Nicotiana tabacum GDP dissociation inhibitor (GDI) mRNA, complete cds. | |
| 700380181H1 | g289757 | 8 | 5 | gb105eukp | ZK353.1 protein | |
| 700346063H1 | g395071 | 15 | 14 | gb105pln | V. faba guanine nucleotide regulatory protein mRNA, complete CDS. | |
| 700342301H1 | g1747418 | 84 | −87 | gb105pln | Z. mays mRNA for eukaryotic initiation factor-5. | |
| 700344234H1 | g2529657 | 16 | −3 | gb105pln | Arabidopsis thaliana chromosome II BAC T30B22 genomic sequence, complete | |
| | | | | | sequence. | |
| 700343577H1 | g2408043 | 42 | −18 | gb105eukp | SPAC29B12.01; hypothetical protein | |
| 700346831H1 | g1666176 | 23 | −1 | gb105pln | N. tabacum mRNA for subunit of NADH:ubiquinone oxidoreductase complex I. | |
| 700346665H1 | g1895083 | 72 | −58 | gb105pln | Zea mays golgi associated protein se-wap41 mRNA, complete cds. | |
| 700343521H1 | g1161311 | 25 | −27 | gb105pln | Arabidopsis thaliana Columbia myo-inositol-1-phosphate synthase mRNA, complete cds. | |
| 700342668H1 | g2668747 | 67 | −51 | gb105pln | Zea mays ribosomal protein L17 (rp117) mRNA, complete cds. | |
| 700346851H1 | g1107484 | 11 | 17 | gb105pln | A. thaliana mRNA for 40S ribosomal protein S15. | |
| 700379988H1 | g1419367 | 24 | −1 | gb105pln | Z. mays ZmABP2 mRNA for actin depolymerizing factor. | |
| 700346058H1 | g899609 | 93 | −82 | gb105pln | Zea mays acidic ribosomal protein P2 (RPA-2A1) mRNA, complete cds. | |
| 700345524H1 | g170099 | 42 | −30 | gb105pln | Spinach betaine-aldehyde dehydrogenase (BADH) mRNA, complete cds. | |
| 700347358H1 | g312178 | 38 | −79 | gb105pln | Z. mays GapC2 gene. | |
| 700345212H1 | g21624 | 37 | −26 | gb105pln | S. vulgare mRNA for glycine-rich RNA-binding protein (clone S2). | |
| 700281969H2 | g2098816 | 10 | 16 | gb105pln | Arabidopsis thaliana BAC F19G10, complete sequence | |
| 700343050H1 | g2842494 | 49 | −30 | gb105eukp | F21O9.200; prohibitin-like protein | |
| 700380548H1 | g683505 | 19 | −1 | gb105pln | A. thaliana gene for 65 kDa regulatory subunit of protein phosphatase 2A. | |
| 700342366H1 | g1419369 | 68 | −62 | gb105pln | Z. mays ZmABP3 mRNA for actin depolymerizing factor. | |
| 700342722H1 | g474832 | 10 | 16 | gb105pln | S. commersonii mRNA for stearoyl-acyl carrier protein desaturase. | |
| 700346272H1 | g1592812 | 13 | 2 | gb105allp | 22 kDa polypeptide | |
| 700343821H1 | g1255684 | 28 | −12 | gb105pln | Rice mRNA for aspartic protease, complete cds. | |
| 700343142H1 | g2766714 | 25 | −1 | gb105pln | Arabidopsis arenosa isolate Care-1(9509) chromomethylase (CMT1) mRNA, | |
| | | | | | partical cds. | |
| 700344717H1 | g2832679 | 32 | −17 | gb104eukp | T10I14.120; putative protein | |
| 700346327H1 | g639494 | 13 | 3 | gb105pln | Zea mays Mu3 transposon in alcohol dehydrogenase (adh1) gene. | |
| 700345243H1 | g1771779 | 16 | −12 | gb105pln | S. tuberosum mRNA for ubiquitin extension protein. | |
| 700342376H1 | g189382 | 62 | −34 | gb105allp | collagenase inhibitor | |
| 700345805H1 | g1694673 | 16 | 2 | gb105allp | proton-ATPase-like protein | |
| 700347239H1 | g1632830 | 20 | 12 | gb105pln | R. communis mRNA (unknown). | |
| 700343842H1 | g1009363 | 14 | 7 | gb105eukp | RGP-3 | |
| 700345262H1 | g829147 | 72 | −61 | gb105pln | Z. mays gene for cyclophilin. | |
| 700345823H1 | g2511690 | 21 | −1 | gb105pln | Phaseolus vulgaris Moldavian encoding cysteine proteinase precursor (clone cp41). | |
| 700343884H1 | g2760844 | 34 | −18 | gb105eukp | F18A8.14 | |
| 700347048H1 | g312180 | 41 | −14 | gb104pln | Z. mays GapC4 gene. | |
| 700347168H1 | g2160155 | 39 | −23 | gb105pln | Sequence of BAC F21M12 from rabidopsis thaliana chromosome 1, complete sequence. | |
| 700344916H1 | g2459406 | 38 | −29 | gb105pln | Arabidopsis thaliana chromosome II BAC F4P9 genomic sequence, complete sequence. | |
| 700343223H1 | g1841501 | 53 | −68 | gb105pln | Z. mays mRNA for glutathione-dependent formaldehyde dehydrogenase. | |
| 700346914H1 | g22272 | 13 | 3 | gb105pln | Maize mRNA for enolase (2-phospho-D-glycerate hydrolase). | |
| 700347341H1 | g558053 | 24 | −3 | gb105pln | Aox1 = alternative oxidase {alternative pathyway} [Nicotiana tabacum = tobacco, | |
| | | | | | cv Bright Yellow, suspension cells, mRNA, 1408 nt]. | |
| 700379729H1 | g416146 | 46 | −35 | gb105pln | Zea mays beta-6 tubulin (tub6) gene and mRNA, complete cds. | |
| 700343618H1 | g20255 | 47 | −22 | gb105pln | O. sativa gene for heat shock protein 82 HSP82. | |
| 700343401H1 | g169820 | 22 | −20 | gb105pln | Oryza sativa triosephosphate isomerase (Rictpi) mRNA, complete cds. | |
| 700379827H1 | g289212 | 48 | −45 | gb105pln | Atriplex nummularia homologous sequence (ANJ1) mRNA. | |
| 700346970H1 | g507278 | 23 | −11 | gb105eukp | involved in cell wall degradation; EC 2.3.1.23 | |
| 700346506H1 | g1845194 | 23 | −43 | gb105pln | Z. mays mRNA for HMGc1 protein. | |
| 700342503H1 | g1136574 | 34 | −35 | gb105pln | Sorghum bicolor heat shock protein 70 (hsp70) pseudogene. | |
| 700379631H1 | g2253156 | 19 | −7 | gb105allp | Ran_GTP binding protein 5 | |
| 700344627H1 | g1181672 | 76 | −63 | gb105pln | Sorghum bicolor heat shock protein 70 cognate (hsc70) mRNA, partial cds. | |
| 700380044H1 | g2301167 | 9 | 2 | gb105allp | unnamed protein product | |
| 700347095H1 | g532816 | 6 | 7 | gb105allp | similar to S. cerevisiae hypothetical 240.3 kd protein in MSH3 3′ region | |
| 700347066H1 | g1765899 | 19 | 5 | gb105eukp | Spot 3 protein | |
| 700346111H1 | g2570504 | 21 | −38 | gb105pln | Oryza sativa proteasome component mRNA, complete cds. | |
| 700345518H1 | g2852444 | 16 | 11 | gb105pln | Salix bakko mRNA for SUI1 homolog, complete cds. | |
| 700344853H1 | g304153 | 8 | 5 | gb105allp | sorbitol dehydrogenase | |
| 700343543H1 | g2264304 | 14 | 8 | gb105pln | Arabidopsis thaliana genomic DNA, chromosome 5, P1 clone: MBG8, complete sequence. | |
| 700343719H1 | g1167801 | 42 | −25 | gb105eukp | F38E11.5 | |
| 700380141H1 | g306451 | 42 | 2 | gb105allp | alpha-tubulin | |
| 700281928H2 | g1086831 | 15 | −10 | gb105eukp | F10E7.7 | |
| 700342268H1 | g532097 | 5 | 6 | gb105allp | weakly similar to S. cerevisiae hypothetical 24.1 kd protein in APN1 3′ region | |
| 700380186H1 | g218176 | 28 | −35 | gb105pln | Rice mRNA for 21kd polypeptide. | |
| 700344889H1 | g170106 | 12 | 14 | gb105pln | Spinach chaperonin 10 (CPN10) mRNA, complete cds. | |
| 700343624H1 | g22324 | 32 | −30 | gb1054pln | Z. mays mRNA for H2B histone (clone cH2B221). | |
| 700346361H1 | g1841869 | 26 | −13 | gb105pln | Pimpinella brachycarpa elongation factor 1-beta (EF-1-beta) mRNA, complete cds. | |
| 700345644H1 | g22277 | 77 | −64 | gb105pln | Maize mRNA for ferritin (clone FM2). | |
| 700346379H1 | g559004 | 32 | −11 | gb105pln | Nicotiana tabacum ascorbate peroxidase (APX) mRNA, complete cds. | |
| 700343746H1 | g1469187 | 8 | 7 | gb105allp | The KIAA0132 gene product is related to Drosophila melanogaster ring canel protein. | |
| 700281968H2 | g1276967 | 11 | −16 | gb105eukp | putative ribosomal protein | |
| 700345450H1 | g599624 | 30 | −2 | gb105pln | A. thaliana mRNA for aconitase (ZAPII). | |
| 700344132H1 | g405882 | 10 | 3 | gb105allp | yeiK | |
| 700345572H1 | g166857 | 34 | −18 | gb105pln | Arabidopsis thaliana cytoplasmic ribosomal protein mRNA, complete cds. | |
| 700380217H1 | g170432 | 14 | 13 | gb105pln | Tomato ATP-dependent protease (CD4A) gene, complete cds. | |
| 700344613H1 | g1754684 | 37 | 0 | gb105eukp | ribosomal protein S23 | |
| 700342902H1 | g168500 | 41 | −52 | gb105pln | Maize (Zea mays) histone H4 gene (H4C14), complete cds. | |
| 700346064H1 | g2688818 | 10 | 5 | gb105allp | Int-6 | |
| 700344548H1 | g2257755 | 51 | −64 | gb105pln | Zea mays nucleolar histone deacetylase HD2-p39 mRNA, complete cds. | |
| 700281952H2 | g940017 | 41 | −45 | gb105pln | Oryza sativa histone H3 gene, complete cds. | |
| 700380796H1 | g533473 | 40 | −30 | gb105pln | Mesembryanthemum crystallinum 2-phospho-D-glycerate hydrolase, enolase, mRNA, | |
| | | | | | complete cds. | |
| 700379736H1 | g886739 | 16 | −10 | gb105pln | Z. mays histone H4 gene. | |
| 700346981H1 | g398605 | 30 | −13 | gb105pln | A. thaliana gene for elongation factor 1 beta. | |
| 700343365H1 | g2668745 | 57 | −49 | gb105pln | Zea mays inorganic pyrophosphatase (IPP) mRNA, complete cds. | |
| 700342484H1 | g1495213 | 12 | 7 | gb105eukp | L1339/SOF1; L1339/SOF1 protein L18-like | |
| 700343618H1 | g170455 | 46 | −21 | gb105pln | Tomato heat shock cognate protein 80 gene, 3′ end. | |
| 700346430H1 | g1185553 | 19 | 5 | gb105pln | Zea mays glyceraldehyde-3-phosphate dehydrogenase (gpc2) gene, | |
| | | | | | complete cds. | |
| 700345943H1 | g1430822 | 12 | 4 | gb105allp | Ste20-like kinase | |
| 700343159H1 | g2213540 | 36 | −8 | gb105eukp | chloroplast DNA-binding protein PD3 | |
| 700380779H1 | g857573 | 38 | −27 | gb105pln | Oryza sativa vacuolar H+-ATPase (vatp-P1) mRNA, complete cds. | |
| 700346118H1 | g520596 | 15 | 3 | gb105eukp | MRE2; Mre2 protein | |
| 700343713H1 | g732951 | 18 | −4 | gb105eukp | SSM1b | |
| 700342483H1 | g2317715 | 36 | −21 | gb105eukp | helD; HelD | |
| 700344603H1 | g182405 | 80 | −44 | gb105allp | farnesyl pyrophosphate synthetase | |
| 700342830H1 | g1546918 | 78 | −76 | gb105pln | Z. mays mRNA for translation initiation factor 5A. | |
| 700342420H1 | g168505 | 32 | −49 | gb105pln | Zea mays histone H3 gene, complete cds. | |
| 700345391H1 | g1877218 | 60 | −50 | gb105pln | H. vulgare mRNA for serine carboxypeptidase III, CP-MIII. | |
| 700344633H1 | g2114206 | 22 | −44 | gb105pln | Rice DNA for glutaredoxin, complete cds. | |
| 700281986H2 | g1946354 | 8 | 16 | gb105pln | Arabidopsis thaliana chromosome II BAC T06B20 genomic sequence, complete | |
| | | | | | sequence. | |
| 700346048H1 | g1009720 | 6 | 6 | gb105eukp | ENOD8; nodulin | |
| 700346829H1 | g563346 | 42 | −18 | gb105eukp | actin-like protein | |
| 700346719H1 | g2852444 | 17 | −10 | gb105pln | Salix bakko mRNA for SUI1 homolog, complete cds. | |
| 700347031H1 | g459894 | 96 | −42 | gb105pln | Zea mays sus1 gene, complete cds. | |
| 700345564H1 | g1263159 | 23 | −22 | gb105pln | O. sativa DNA for LRK2 gene. | |
| 700344116H1 | g950052 | 22 | −25 | gb105pln | H. vulgare mRNA for HMG1/2-like protein. | |
| 700343713H1 | g1370456 | 18 | −4 | gb105eukp | SSM1 | |
| 700342385H1 | g1632831 | 12 | 7 | gb105eukp | orf | |
| 700281860H2 | g168492 | 63 | 12 | gb105pln | Corn histone H3 (H3C3) gene, complete cds. | |
| 700346535H1 | g1519250 | 46 | −32 | gb105pln | Oryza sativa GF14-c protein mRNA, complete cds. | |
| 700344632H1 | g1199863 | 20 | −3 | gb105eukp | NEX-1; calcium dependent lipid-binding protein; NEX1 annexin | |
| 700343627H1 | g166931 | 22 | 11 | gb105pln | A. thaliana ubiquitin extension protein (UBQ2) gene, complete cds. | |
| 700380168H1 | g2388661 | 49 | −33 | gb105pln | Lolium perenne cinnamyl alcohol dehydrogenase mRNA, complete cds. | |
| 700345605H1 | g2407800 | 26 | −5 | gb105eukp | H2Av; histone H2A.F/Z | |
| 700344696H1 | g205858 | 16 | 8 | gb105allp | CDS is longest ORF of a cDNA clone which hybridizes to a 1.2 kb transcript present | |
| | | | | | at higher levels in the ventral prostate of 3-day castrate than in normal rats | |
| 700346007H1 | g2827552 | 7 | 4 | gb105eukp | T12H17.140; predicted protein | |
| 700346303H1 | g2341023 | 21 | 3 | gb105pln | Sequence of BAC F19P19 from Arabidopsis thaliana chromosome 1, complete sequence. | |
| 700344965H1 | g2465923 | 19 | −4 | gb105eukp | RKF1; receptor-like serine/threonine kinase | |
| 700342556H1 | g1402874 | 11 | 9 | gb105pln | A. thaliana 81 kb genomic sequence. | |
| 700343710H1 | g1184775 | 56 | −42 | gb105pln | Zea mays glyceraldehyde-3-phosphate dehydrogenase GAPC4 (gpc4) mRNA, | |
| | | | | | complete cds. | |
| 700344327H1 | g1747418 | 51 | −71 | gb105pln | Z. mays mRNA for eukaryotic initiation factor-5. | |
| 700379624H1 | g950052 | 25 | −31 | gb105pln | H. vulgare mRNA for HMG1/2-like protein. | |
| 700345312H1 | g1785621 | 18 | −7 | gb105eukp | putative serine/threonine protein kinase; AtPK2324 | |
| 700346839H1 | g1707370 | 17 | 6 | gb105eukp | RSp41; splicing factor | |
| 700346681H1 | g397274 | 19 | 4 | gb105allp | Tonoplast intrinsic protein | |
| 700380593H1 | g168462 | 37 | −40 | gb105pln | Zea mays defective suppressor-mutator from the brittle-1-mutable allele. | |
| 700346025H1 | g1825645 | 38 | −17 | gb105eukp | F46F11.4 | |
| 700345985H1 | g556559 | 33 | −17 | gb105pln | Rice mRNA for homologue of Tat binding protein, complete cds. | |
| 700345946H1 | g289658 | 47 | −26 | gb105eukp | C50C3.6 protein | |
| 700343878H1 | g2668743 | 62 | −29 | gb105pln | Zea mays ubiquitin conjugating enzyme (UBC) mRNA, complete cds. | |
| 700380569H1 | g2407800 | 24 | −1 | gb105eukp | H2Av; histone H2A.F/Z | |
| 700342869H1 | g56492 | 8 | 3 | gb105allp | insulin-degrading enzyme | |
| 700281872H2 | g511665 | 58 | −3 | gb105pln | Rice gene for aspartic protease, complete cds. | |
| 700345785H1 | g1469187 | 7 | 6 | gb105allp | The KIAA0132 gene product is related to Drosophila melanogaster ring canel protein. | |
| 700342554H1 | g1296666 | 22 | 8 | gb105allp | Sec23 protein | |
| 700379824H1 | g1044912 | 27 | 6 | gb105eukp | ribonucleotide reductase R2 | |
| 700342862H1 | g829147 | 96 | −24 | gb105pln | Z. mays gene for cyclophilin. | |
| 700345055H1 | g1041674 | 74 | −40 | gb105pln | Z. mays mRNA for V-type H+-ATPase, clone 16-2. | |
| 700344290H1 | g2345153 | 76 | −73 | gb105pln | Zea mays ribsomal protein S4 (rps4) mRNA, complete cds. | |
| 700343350H1 | g473604 | 27 | −23 | gb105pln | Zea mays W-22 histone H2B mRNA, complete cds. | |
| 700345155H1 | g1498486 | 22 | 6 | gb105allp | thioredoxin reductase | |
| 700379770H1 | g2760173 | 40 | −12 | gb105pln | Arabidopsis thaliana genomic DNA, chromosome 5, P1 clone: MYH19, | |
| | | | | | complete sequence. | |
| 700346549H1 | g2330821 | 13 | 6 | gb105allp | hypothetical protein | |
| 700281968H2 | g1669623 | 15 | −6 | gb105eukp | ribosomal protein L39 | |
| 700342220H1 | g1129084 | 43 | −34 | gb105pln | Wheat mRNA for protein H2A, complete cds, clone wcH2A-3. | |
| 700345821H1 | g1550813 | 86 | −67 | gb105pln | Z. mays mRNA for acidic robosomal protein P0. | |
| 700345008H1 | g1834353 | 10 | 5 | gb105eukp | 194; hypothetical protein 194 | |
| 700346979H1 | g161631 | 14 | 1 | gb105eukp | 217 g protein | |
| 700344804H1 | g463251 | 38 | −24 | gb105pln | M. sativa (Nagyszenasi) mRNA for ribosomal protein RL5. | |
| 700343507H1 | g747914 | 56 | −41 | gb105pln | Z. mays CaM1 mRNA for calmodulin. | |
| 700347206H1 | g168545 | 62 | −23 | gb105pln | Z. mays putative ribosomal protein L19 homolog mRNA, partial cds. | |
| 700281987H2 | g1008880 | 19 | 6 | gb105pln | Phaseolus vulgaris eukaryotic initiation factor 5 (eIF-5) gene, | |
| | | | | | complete cds. | |
| 700379753H1 | g2281115 | 49 | −18 | gb105eukp | T10P11.14.1; putative cullin-like 1 protein | |
| 700342476H1 | g473604 | 35 | −17 | gb105pln | Zea mays W-22 histone H2B mRNA, complete cds. | |
| 700342869H1 | g157168 | 8 | 4 | gb105allp | insulin-degrading enzyme | |
| 700346527H1 | g699510 | 10 | −4 | gb105eukp | M(1)1B; minute(1)1B protein; minute(1)1B | |
| 700345923H1 | g2252639 | 28 | −9 | gb105pln | Genomic sequence of Arabidopsis BAC F8A5, complete sequence. | |
| 700343205H1 | g625146 | 6 | 3 | gb105allp | Ro protein | |
| 700346196H1 | g602605 | 48 | −65 | gb105pln | Zea mays tandem genes for alpha1-tubulin and alpha2-tubulin. | |
| 700345971H1 | g1184773 | 74 | −80 | gb105pln | Zea mays glyceraldehyde-3-phosphate dehydrogenase GAPC3 (gpc3) mRNA, | |
| | | | | | complete cds. | |
| 700345452H1 | g587542 | 12 | 12 | gb105pln | Yeast (Saccharomyces cerevisiae) PRS3 gene for ribose-phosphate pyrophosphokinase. | |
| 700344311H1 | g2760173 | 29 | −7 | gb105pln | Arabidopsis thaliana genomic DNA, chromosome 5, P1 clone: MYH19, complete | |
| | | | | | sequence. | |
| 700347072H1 | g2340104 | 17 | 5 | gb105allp | Very similar and perhaps identical to Hs-CUL-4B.; 80-100% similarity to partial | |
| | | | | | sequence U58091 (PID:g1381150). | |
| 700346344H1 | g2058311 | 18 | −6 | gb105eukp | CCR; cinnamoyl-CoA reductase; EC 1.2.1.44 | |
| 700346340H1 | g1246472 | 20 | 4 | gb105allp | R102.4 | |
| 700347368H1 | g488835 | 8 | 6 | gb105allp | ribosomal protein L28 | |
| 700344876H1 | g2564051 | 15 | 8 | gb105pln | Arabidopsis thaliana genomic DNA, chromosome 5, P1 clone: MWD9, complete | |
| | | | | | sequence. | |
| 700343384H1 | g22174 | 55 | −53 | gb105pln | Maize Aux311 gene for auxin-binding protein. | |
| 700342309H1 | g1151003 | 9 | 5 | gb105allp | hypothetical protein UNF346 | |
| 700344204H1 | g2565420 | 17 | 1 | gb105pln | Onobrychis viciifolia eukaryotic translation initiation factor eIF-1A mRNA, | |
| | | | | | complete cds. | |
| 700346443H1 | g19103 | 28 | −12 | gb105pln | H. vulgare mRNA for ribosomal protein L17-2. | |
| 700343625H1 | g289156 | 21 | −5 | gb105allp | amine oxidase | |
| 700345132H1 | g168543 | 78 | −24 | gb105pln | Zea mays putative ribosomal protein S8 mRNA, partial cds. | |
| 700346114H1 | g294518 | 27 | −10 | gb105allp | annexin II | |
| 700345668H1 | g173317 | 21 | −8 | gb105eukp | RPL30; ribosomal protein L30; L30 | |
| 700344276H1 | g1336096 | 27 | −6 | gb105pln | Pisum sativum pyruvate dehydrogenase E1beta mRNA, nuclear gene encoding | |
| | | | | | mitochondrial protein, complete cds. | |
| 700344173H1 | g312178 | 37 | −65 | gb105pln | Z. mays GapC2 gene. | |
| 700345946H1 | g395157 | 21 | −6 | gb105eukp | PRP8; pre-mRNA splicing factor, U5 snRNP protein; PRP8 | |
| 700344686H1 | g2687685 | 10 | 16 | gb105pln | Arabidopsis thaliana cys gene. | |
| 700343434H1 | g1302303 | 9 | −8 | gb105eukp | ORF YNL255c | |
| 700346353H1 | g2653284 | 26 | −21 | gb105pln | Oryza sativa mRNA for enoyl-ACP reductase. | |
| 700343035H1 | g300249 | 13 | 7 | gb105allp | DMR-N9 {C-terminal} [mice, brain, Peptide Partial, 568 aa] | |
| 700344203H1 | g602302 | 19 | −1 | gb105eukp | DIM; cell elongation; diminuto | |
| 700380088H1 | g1431253 | 29 | −12 | gb105pln | Yeast (Saccharomyces cerevisiae) chromosome IV reading frame ORF YDL160c. | |
| 700380462H1 | g473602 | 47 | −38 | gb105pln | Zea mays W-22 histone H2A mRNA, complete cds. | |
| 700347138H1 | g1136121 | 71 | −61 | gb105pln | O. sativa mRNA for alpha-tubulin (clone OSTA-136). | |
| 700345087H1 | g18259 | 43 | −32 | gb105pln | C. sativus mRNA for cs DnaJ-1. | |
| 700343661H1 | g1778820 | 25 | −19 | gb105pln | Oryza sativa S-adenosyl-L-methionine synthetase (pOS-SAMS2) mRNA, complete cds. | |
| 700343150H1 | g603951 | 6 | 7 | gb105allp | This gene is novel. | |
| 700380138H1 | g1658321 | 24 | −10 | gb105pln | S. tuberosum mRNA for transsketolase. | |
| 700346602H1 | g1154953 | 46 | −55 | gb105pln | T. aestivum histone H2A gene. | |
| 700345489H1 | g405612 | 23 | −20 | gb105pln | A. thaliana ribosomal protein gene S18, complete CDS. | |
| 700344259H1 | g1301977 | 11 | −2 | gb104eukp | PMS1 | |
| 700344036H1 | g459894 | 74 | −30 | gb105pln | Zea mays sus1 gene, complete cds. | |
| 700345422H1 | g2145356 | 19 | −10 | gb105eukp | ATHB-14; HD-Zip protein | |
| 700345225H1 | g683503 | 24 | −3 | gb105pln | A. thaliana mRNA for 65 kDa regulatory subunit of protein phosphatase 2A. | |
| 700344448H1 | g20760 | 18 | 13 | gb105pln | P. sativum gTUB3 mRNA for beta-tubulin 3. | |
| 700343564H1 | g1167955 | 8 | −1 | gb105eukp | putative 32.7 kDa jasmonate-induced protein | |
| 700343701H1 | g21832 | 26 | −15 | gb105pln | Wheat mRNA for chloroplast phosphoglycerate kinase (EC 2.7.2.3). | |
| 700345724H1 | g736271 | 72 | −75 | gb105pln | O. sativa hsp70 gene for heat shock protein 70. | |
| 700342714H1 | g2275006 | 35 | −31 | gb105pln | Hordeum vulgare mRNA for expressed sequence tag. | |
| 700380608H1 | g664900 | 32 | −26 | gb105pln | C. plantagineum tkt3 gene for transketolase. | |
| 700346419H1 | g426441 | 24 | −3 | gb105pln | Rice mRNA for thioredoxin h, complete cds. | |
| 700343439H1 | g168500 | 75 | −59 | gb105pln | Maize (Zea mays) histone H4 gene (H4C14), complete cds. | |
| 700347241H1 | g493709 | 63 | −59 | gb105pln | Rice mRNA for beta-tubulin, complete cds. | |
| 700346110H1 | g17677 | 24 | −11 | gb105pln | Arabidopsis thaliana UBQ4 gene for polyubiquitin. | |
| 700345483H1 | g2058311 | 15 | −1 | gb105eukp | CCR; cinnamoyl-CoA reductase; EC 1.2.1.44 | |
| 700345209H1 | g416146 | 78 | −75 | gb105pln | Zea mays beta-6 tubulin (tub6) gene and mRNA, complete cds. | |
| 700343338H1 | g2392023 | 42 | −20 | gb105allp | Acyl-CoA synthetase | |
| 700345962H1 | g2564044 | 15 | 10 | gb105pln | Arabidopsis thaliana genomic DNA, chromosome 5, TAC clone: K19P17, complete | |
| | | | | | sequence. | |
| 700345488H1 | g2351063 | 19 | −2 | gb105pln | Arabidopsis thaliana genomic DNA, chromosome 5, P1 clone: MCL19. | |
| 700344214H1 | g21834 | 23 | −24 | gb105pln | Wheat mRNA for cytosolic phosphoglycerate kinase (EC 2.7.2.3). | |
| 700342888H1 | g288614 | 48 | −34 | gb105pln | D. carota mRNA for dihydrofolate reductase and thymidylate synthetase. | |
| 700346886H1 | g474009 | 67 | −47 | gb105pln | Rice mRNA, partial homologous to ribosomal protein S19 gene. | |
| 700343702H1 | g1088344 | 8 | 5 | gb105allp | adenylate kinase | |
| 700346630H1 | g1199479 | 14 | 17 | gb105pln | Cucumis sativus mRNA, clone CRR48, homologous to Arabidopsis cDNA clone 3003. | |
| 700345732H1 | g1336096 | 21 | −3 | gb105pln | Pisum sativum pyruvate dehydrogenase Elbeta mRNA, nuclear gene encoding | |
| | | | | | mitochondrial protein, complete cds. | |
| 700344205H1 | g310932 | 24 | −34 | gb105pln | Nicotiana tabacum ribosomal protein L17 mRNA, complete cds. | |
| 700342925H1 | g1049252 | 47 | −5 | gb105pln | Zea mays vacuolar ATPase 69 kDa subunit mRNA, partial cds. | |
| 700347084H1 | g21800 | 35 | −31 | gb105pln | T. aestivum L mRNA for histone H2B. | |
| 700343138H1 | g1064930 | 18 | −6 | gb105pln | N. tabacum mRNA for cyclin A-like protein (clone 59). | |
| 700344259H1 | g791102 | 11 | −2 | gb105eukp | PMS1; DNA mismatch repair protein | |
| 700380266H1 | g639494 | 22 | −6 | gb105pln | Zea mays Mu3 transposon in alcohol dehydrogenase (adh1) gene. | |
| 700345990H1 | g2558938 | 13 | 7 | gb105allp | arm repeat containing protein | |
| 700343318H1 | g171704 | 19 | −3 | gb105eukp | COO1 | |
| 700347263H1 | g606969 | 11 | 17 | gb105pln | Arabidopsis thaliana cytoplasmic gb105eukp | lp-ipmsa; first enzyme in leucine |
| | | | | | biosynthesis which catalyzes condensation of acetyl-CoA and 2-oxoisovalerate to form | |
| | | | | | 2-isopropylmalate synthase; 2-isopropylmalate synthase; EC 4.1.3.12 | |
| 700379935H1 | g550437 | 25 | −8 | gb105pln | Z. mays CYP71C2 mRNA for cytochrome P-450. | |
| 700344711H1 | g2511530 | 46 | −50 | gb105pln | Eleusine indica alpha tubulin 1 (TUA1) mRNA, complete cds. | |
| 700347014H1 | g2662342 | 71 | −25 | gb105pln | Oryza sativa mRNA for EF-1 alpha, complete cds. | |
| 700344536H1 | g2160155 | 13 | 16 | gb105pln | Sequence of BAC F21M12 from Arabidopsis thaliana chromosome 1, complete | |
| | | | | | sequence. | |
| 700344425H1 | g1381148 | 23 | 2 | gb105allp | Hs-CUL-4A | |
| 700380144H1 | g1881694 | 73 | −67 | gb105pln | Zea mays phosphoglucomutase mRNA, partial cds. | |
| 700346096H1 | g21794 | 44 | 6 | gb105pln | Wheat histone H4 gene. | |
| 700343810H1 | g313759 | 100 | −90 | gb105pln | Z. mays hsp 70-1 gene for heat shock protein 70. | |
| 700342782H1 | g1617270 | 39 | −22 | gb105eukp | acyl-CoA synthetase; EC 6.2.1.3 | |
| 700344792H1 | g1174333 | 10 | 4 | gb105allp | w-amino-transferase-like protein | |
| 700343480H1 | g1276967 | 13 | 6 | gb105allp | putative ribosomal protein | |
| 700345969H1 | g644492 | 49 | 13 | gb105pln | Corn elongation factor 1alpha gene, complete cds. | |
| 700343894H1 | g1136119 | 74 | −36 | gb105pln | O. sativa mRNA for alpha-tubulin (clone OSTA-111). | |
| 700342671H1 | g486279 | 12 | 4 | gb105eukp | ORF YKL160w | |
| 700346327H1 | g2317907 | 15 | 3 | gb105allp | Mago Nashi-like protein | |
| 700345334H1 | g968902 | 20 | 7 | gb105eukp | ribosomal protein S8 | |
| 700342869H1 | g2291181 | 9 | 5 | gb105allp | strong similarity to peptidase family M16 or the insulinase family | |
| 700344420H1 | g450548 | 28 | −27 | gb105pln | O. sativa (pRSAM-1) gene for S-adenosyl methionine synthetase. | |
| 700345990H1 | g2462822 | 24 | 3 | gb105eukp | F10G19.3 | |
| 700343761H1 | g2414643 | 29 | −12 | gb105pln | Yeast (Saccharomyces pombe) chromosome I cosmid c3H5. | |
| 700344179H1 | g2347207 | 13 | −2 | gb105eukp | T09D09.3; APG protein isolog | |
| 700343287H1 | g939780 | 49 | −29 | gb105pln | Zea mays MADS-box protein (ZAG5) mRNA, complete cds. | |
| 700347080H1 | g2599092 | 32 | 6 | gb105eukp | MSI4; WD-40 repeat protein MSI4 | |
| 700346811H1 | g303856 | 49 | −32 | gb105pln | Rice mRNA for ubiquitin protein fused to a ribosomal protein, complete cds. | |
| 700346845H1 | g1877414 | 24 | −0 | gb105allp | XSUG1 | |
| 700346726H1 | g854688 | 27 | −6 | gb105eukp | cytochrome c oxidase subunit I | |
| 700343934H1 | g21800 | 37 | −36 | gb105pln | T. aestivum L mRNA for histone H2B. | |
| 700342491H1 | g2108346 | 39 | −36 | gb105pln | Brassica campestris small GTP-binding protein Bsar1b (bsar1b) mRNA, complete cds. | |
| 700379854H1 | g2564045 | 12 | 15 | gb105pln | Arabidopsis thaliana genomic DNA, chromosome 5, TAC clone: K8K14, complete | |
| | | | | | sequence. | |
| 700346720H1 | g2209357 | 21 | −3 | gb105pln | Mangifera indica beta-D-galactosidase (SP26) mRNA, partial cds. | |
| 700345424H1 | g168396 | 25 | −1 | gb105pln | Zea mays auxin-binding protein (abp1) gene, exons 1-5 and complete cds. | |
| 700345337H1 | g218142 | 36 | −25 | gb105pln | Rice mRNA for aspartic proteinase, complete cds. | |
| 700344696H1 | g2459928 | 16 | 8 | gb105allp | rat ventral prostate.1 homolog | |
| 700380638H1 | g1841501 | 66 | −70 | gb105pln | Z. mays mRNA for glutathione-dependent formaldehyde dehydrogenase. | |
| 700345741H1 | g488712 | 9 | 4 | gb105eukp | cr7; ubiquinol-cytochrome c reductase; EC 1.10.2.2 | |
| 700345514H1 | g304153 | 15 | −3 | gb105allp | sorbitol dehydrogenase | |
| 700345281H1 | g829147 | 79 | −72 | gb105pln | Z. mays gene for cyclophilin. | |
| 700342577H1 | g63752 | 17 | 1 | gb105allp | PR 264 | |
| 700346120H1 | g1946668 | 12 | −4 | gb105eukp | COII; cytochrome c oxidase subunit II | |
| 700344689H1 | g475257 | 38 | 4 | gb105allp | similar to ribosomal protein L19 | |
| 700343702H1 | g216516 | 9 | 4 | gb105allp | adenylate kinase | |
| 700346017H1 | g2582063 | 65 | 2 | gb105allp | NADH dehydrogenase subunit 2 | |
| 70345522H1 | g398921 | 23 | −2 | gb105pln | B. napus cold induced protein (BnC24B). | |
| 700343821H1 | g218142 | 41 | −27 | gb105pln | Rice mRNA for aspartic proteinase, complete cds. | |
| 700379606H1 | g1419448 | 10 | 2 | gb105eukp | K04G2.5 | |
| 700346009H1 | g303852 | 61 | −50 | gb105pln | Rice mRNA for ribosomal protein L3, complete cds. | |
| 700345577H1 | g1015315 | 24 | 5 | gb105pln | Pisum sativum (clone PsRCI35-2) ribosomal protein L41 mRNA, complete cds. | |
| 700344759H1 | g2252834 | 8 | 5 | gb105eukp | A_IG005I10.14 | |
| 700345894H1 | g433608 | 28 | −5 | gb105pln | R. communis mRNA for enolase. | |
| 700342829H1 | g2828186 | 26 | −6 | gb105pln | Arabidopsis thaliana genomic DNA, chromosome 5, TAC clone: K18I23, complete | |
| | | | | | sequence. | |
| 700345225H1 | g437900 | 24 | −3 | gb105pln | P. sativum mRNA for phosphoprotein phosphatase 2A 65 kDa regulatory subunit. | |
| 700343582H1 | g22469 | 26 | −15 | gb105pln | Maize mRNA for cytoplasmic ribosomal protein S11. | |
| 700343434H1 | g159342 | 6 | −8 | gb105eukp | HEXBP; HEXBP DNA binding protein | |
| 700346366H1 | g1399567 | 25 | −45 | gb105pln | Podophyllum peltatum nuclear 26S ribosomal RNA gene, partial sequence. | |
| 700346117H1 | g2435604 | 33 | −16 | gb105eukp | F08F1.7 | |
| 700344237H1 | g2276349 | 26 | −3 | gb105pln | Yeast (Saccharomyces pombe) chromosome II cosmid c30D10. | |
| 700379706H1 | g2407800 | 17 | −2 | gb105eukp | H2Av; histone H2A.F/Z | |
| 700346931H1 | g1055130 | 17 | −3 | gb105eukp | ZK622.3 | |
| 700346859H1 | g895891 | 14 | −2 | gb105eukp | RPS5; ribosomal protein S5 | |
| 700345259H1 | g1580810 | 6 | 1 | gb105eukp | Sm10; T-cell-stimulating antigen | |
| 700380244H1 | g508974 | 65 | −59 | gb105pln | Triticum aestivum Chinese spring protein disulfide isomerase (PDI) mRNA, | |
| | | | | | complete cds. | |
| 700345908H1 | g2226414 | 13 | 7 | gb105eukp | SPBC31F10.03; hypothetical protein | |
| 700344438H1 | g536151 | 14 | 17 | gb105pln | Yeast (Saccharomyces cerevisiae) chromosome II reading frame ORF YBL092w. | |
| 700343918H1 | g488779 | 60 | −53 | gb105pln | T. aestivum mRNA for isomerase. | |
| 700380595H1 | g609261 | 68 | −59 | gb105pln | S. cereale (cv. Halo) mRNA for triosephosphate isomerase. | |
| 700345766H1 | g290056 | 32 | −0 | gb105pln | Dictyostelium discoideum HIV1 TAT-binding protein homologue (TBP1O) mRNA, | |
| | | | | | 3′ end. | |
| 700344655H1 | g2394228 | 27 | 11 | gb105pln | Arabidopsis thaliana WD-40 repeat protein (MSI1) mRNA, complete cds. | |
| 700346681H1 | g1632821 | 24 | 3 | gb105pln | O. sativa mRNA for transmembrane protein. | |
| 700345706H1 | g1375075 | 17 | 5 | gb105eukp | glyoxysomal malate dehydrogenase; EC 1.1.137 | |
| 700344162H1 | g2511532 | 29 | −3 | gb105pln | Eleusine indica alpha-tubulin 2 (TUA2) mRNA, complete cds. | |
| 700342644H1 | g1326286 | 7 | 3 | gb105eukp | T27A10.3 | |
| 700380515H1 | g2708493 | 5 | 7 | gb105allp | synaptojanin II | |
| 700345514H1 | g1742885 | 13 | −1 | gb105allp | Sorbitol dehydrogenase (EC 1.1.1.14) (L-iditol 2-dehydrogenase). | |
| 700347127H1 | g20163 | 39 | −5 | gb105pln | O. sativa Rr15 mRNA for 5S ribosomal RNA. | |
| 700344426H1 | g28519 | 32 | −5 | gb105allp | L apoferritin | |
| 700347284H1 | g1372966 | 31 | −5 | gb105eukp | CREB-like protein | |
| 700345077H1 | g168795 | 19 | −2 | gb105pln | Neurospora crassa ribosomal protein gene, complete cds. | |
| 700345427H1 | g2760173 | 29 | −12 | gb105pln | Arabidopsis thaliana genomic DNA, chromosome 5, P1 clone: MYH19, complete | |
| | | | | | sequence. | |
| 700346527H1 | g458972 | 11 | −7 | gb105eukp | F37C12.4 | |
| 700343157H1 | g644491 | 80 | −79 | gb105pln | Corn mRNA for elongation factor 1A. | |
| 700346724H1 | g2827635 | 13 | 6 | gb105eukp | F10N7.170; predicted protein | |
| 700342267H1 | g1184775 | 86 | −76 | gb105pln | Zea mays glyceraldehyde-3-phosphate dehydrogenase GAPC4 (gpc4) mRNA, | |
| | | | | | complete cds. | |
| 700343277H1 | g2511690 | 20 | −1 | gb105pln | Phaseolus vulgaris Moldavian encoding cysteine proteinase precursor (clone cp41). | |
| 700342208H1 | g899609 | 17 | −15 | gb105pln | Zea mays acidic ribosomal protein P2 (RPA-2A1) mRNA, complete cds. | |
| 700345065H1 | g20255 | 55 | −66 | gb105pln | O. sativa gene for heat shock protein 82 HSP82. | |
| 700343253H1 | g2668737 | 60 | −59 | gb105pln | Zea mays translation initiation factor 5A (TIF5A) mRNA, complete cds. | |
| 700342593H1 | g22469 | 66 | −44 | gb105pln | Maize mRNA for cytoplasmic ribosomal protein S11. | |
| 700342482H1 | g169588 | 11 | 13 | gb105pln | Solanum tuberosum spliceosomal protein (U2B) mRNA, complete cds. | |
| 700345667H1 | g2331300 | 56 | −53 | gb105pln | Zea mays ribosomal protein S4 type I (rps4) mRNA, complete cds. | |
| 700380090H1 | g1054718 | 30 | −3 | gb105eukp | coxIII; cytochrome oxidase | |
| 700346819H1 | g2462748 | 20 | −15 | gb105eukp | F8A5.29; putative Clathrin Coat Assembly protein | |
| 700281884H2 | g1185555 | 17 | 17 | gb105pln | Zea mays glyceraldehyde-3-phosphate dehydrogenase (gpc4) gene, partial cds. | |
| 700346229H1 | g166687 | 47 | −39 | gb105pln | A. thaliana 3-deoxy-D-arabino-heptulosonate y-phosphate synthase (DHS1) mRNA, | |
| | | | | | complete cds. | |
| 700345244H1 | g1947005 | 12 | 5 | gb105eukp | T21G5.5 | |
| 700343696H1 | g2239142 | 17 | 5 | gb105allp | polycomb-M33 interacting protein Ring1B | |
| 700379891H1 | g1053052 | 17 | −27 | gb105pln | Hordeum vulgare histone H3 gene, partial cds, clone B1. | |
| 700346875H1 | g2462828 | 23 | −6 | gb105eukp | F19G10.9 | |
| 700345693H1 | g1184773 | 77 | −75 | gb105pln | Zea mays glyceraldehyde-3-phosphate dehydrogenase GAPC3 (gpc3) mRNA, | |
| | | | | | complete cds. | |
| 700346269H1 | g161631 | 8 | −3 | gb105eukp | 217 g protein | |
| 700343108H1 | g218099 | 32 | −44 | gb105pln | Rice mRNA for ribosomal protein S12 (320 gene), partial sequence. | |
| 700281908H2 | g2622711 | 9 | 7 | gb105allp | ferripyochelin binding protein | |
| 700342270H1 | g2286152 | 50 | −34 | gb105pln | Zea mays cytoplasmic malate dehydrogenase mRNA, complete cds. | |
| 700342379H1 | g527680 | 38 | −14 | gb105eukp | ribosomal protein S3 | |
| 700379969H1 | g1488267 | 17 | 6 | gb105eukp | low affinity calcium antiporter CAX2 | |
| 700347051H1 | g1149570 | 17 | −7 | gb105pln | A. thaliana mRNA for mitochondrial elongation factor Tu. | |
| 700346705H1 | g22322 | 22 | −22 | gb105pln | Z. mays mRNA for H2B histone (clone cH2B214). | |
| 700342214H1 | g886679 | 11 | 4 | gb105eukp | LeUBC1; ubiquitin conjugating enzyme | |
| 700346213H1 | g2624200 | 35 | −31 | gb105pln | M. acuminata mRNA; clone pBAN UU91. | |
| 700343567H1 | g2623679 | 21 | −14 | gb105pln | Zea mays calmodulin (Zmrcalm) mRNA, complete cds. | |
| 700346414H1 | g544506 | 7 | 4 | gb105eukp | SIK1; Sik1p | |
| 700347051H1 | g218309 | 12 | 4 | gb105pln | N. sylvestris tufA gene for chloroplast elongation factor TuA (EF-TuA), partial sequence. | |
| 700345438H1 | g2665889 | 45 | −32 | gb105pln | Fragaria x ananassa calcium-dependent protein kinase (MAX17) mRNA, complete cds. | |
| 700344542H1 | g216201 | 7 | 7 | gb105allp | prolyl endopeptidase | |
| 700380031H1 | g166786 | 31 | 8 | gb105eukp | PAB5; poly(A)-binding protein | |
| 700346439H1 | g1431108 | 26 | −11 | gb105eukp | ORF YDL084w | |
| 700343716H1 | g514945 | 87 | −74 | gb105pln | Zea mays sucrose synthase (Sus1) mRNA, complete cds. | |
| 700343128H1 | g1325967 | 16 | 8 | gb105pln | T. aestivum histone H2A gene (clone TH274). | |
| 700346266H1 | g438279 | 14 | 3 | gb105allp | Ribosomal protein L7 | |
| 700342745H1 | g430992 | 20 | −9 | gb105eukp | protein kinase | |
| 700380595H1 | g806311 | 38 | −28 | gb105pln | Spinacia oleracea nuclear encoded triosephosphate isomerase, chloroplast isozyme | |
| | | | | | mRNA, complete cds. | |
| 700346018H1 | g8159 | 29 | −16 | gb105eukp | Kr; Kr polypeptide | |
| 700345164H1 | g1419909 | 10 | 5 | gb105eukp | ORF YOL077c | |
| 700344063H1 | g550543 | 41 | −35 | gb105pln | A. thaliana mRNA for ribosomal protein L16. | |
| 700379706H1 | g2564109 | 7 | 7 | gb105eukp | histone H2A | |
| 700343762H1 | g556018 | 30 | −13 | gb105pln | Barley glutamate 1-semialdehyde aminotransferase (GSA) mRNA, complete cds. | |
| 700343254H1 | g2435511 | 27 | −20 | gb105eukp | A_TM017A05.10 | |
| 700342945H1 | g602302 | 27 | −4 | gb105eukp | DIM; cell elongation; diminuto | |
| 700345760H1 | g2618602 | 28 | 12 | gb105pln | Arabidopsis thaliana genomic DNA, chromosome 5, P1 clone: MSJ1, complete | |
| | | | | | sequence. | |
| 700344609H1 | g1732514 | 10 | 16 | gb105pln | Arabidopsis thaliana myosin heavy chain-like protein mRNA, complete cds. | |
| 700342411H1 | g2330808 | 18 | −10 | gb105eukp | Sum1p | |
| 700347006H1 | g167099 | 18 | 4 | gb105pln | Hordeum vulgare seed imbitition protein (Sip1) gene, complete cds. | |
| 700345986H1 | g2289094 | 15 | 6 | gb105pln | Arabidopsis thaliana WD-40 repeat protein (AtArcA) mRNA, complete cds. | |
| 700344228H1 | g473602 | 61 | −69 | gb105pln | Zea mays W-22 histone H2A mRNA, complete cds. | |
| 700343593H1 | g541611 | 33 | −16 | gb105allp | cytokeratin 13 | |
| 700343291H1 | g2408032 | 11 | 5 | gb105eukp | SPAC17G6.19c; hypothetical protein | |
| 700380261H1 | g510238 | 28 | −13 | gb105eukp | OPT1-1; oligopeptide transporter 1-1 | |
| 700344915H1 | g2505874 | 26 | −12 | gb105eukp | putative kinase | |
| 700342692H1 | g11531 | 34 | −21 | gb105pln | E. gracilis chloroplast gene for elongation factor Tu. | |
| 700346682H1 | g1183936 | 15 | 4 | gb105pln | P. sativum 5S rRNA gene. | |
| 700345302H1 | g914876 | 19 | −1 | gb105eukp | unknown | |
| 700380508H1 | g1841463 | 19 | −2 | gb105pln | N. tabacum mRNA for LIM-domain protein. | |
| 700345525H1 | g2827624 | 15 | 8 | gb105eukp | hypothetical protein | |
| 700343577H1 | g1322734 | 36 | −17 | gb105eukp | ORF YGL150c | |
| 700345984H1 | g886739 | 64 | −12 | gb105pln | Z. mays histone H4 gene. | |
| 700345985H1 | g732814 | 21 | 2 | gb105pln | L. esculentum LeMA-1 mRNA for putative Mg-dependent ATPase 1. | |
| 700345318H1 | g1235680 | 7 | 7 | gb105allp | receptor serine/threonine kinase PR5K | |
| 700342823H1 | g485376 | 78 | −77 | gb105pln | Zea mays alpha-3-tubulin gene, complete cds. | |
| 700346435H1 | g2465923 | 6 | 3 | gb105eukp | RKF1; receptor-like serine/threonine kinase | |
| 700342734H1 | g1841869 | 43 | −38 | gb105pln | Pimpinella brachycarpa elongation factor 1-beta (EF-1-beta) mRNA, complete cds. | |
| 700347177H1 | g2347195 | 9 | 5 | gb105eukp | T09D09.11 | |
| 700344831H1 | g2773184 | 21 | −2 | gb105eukp | F33D11.10 | |
| 700344310H1 | g2464877 | 51 | −25 | gb105eukp | methionyl aminopeptidase homolog | |
| 700345783H1 | g2464854 | 24 | −9 | gb105eukp | Cu2+-transporting ATPase homolog | |
| 700379826H1 | g1049306 | 39 | −25 | gb105pln | Arabidopsis thaliana actin-2 mRNA, complete cds. | |
| 700346945H1 | g20000 | 41 | −29 | gb105pln | N. tabacum RL2 mRNA for 60S ribosomal protein L2. | |
| 700342465H1 | g971862 | 26 | −7 | gb105allp | potential laminin-binding protein | |
| 700379817H1 | g168490 | 26 | 2 | gb105pln | Maize glutathione S-transferase (GST-I) mRNA, complete cds. | |
| 700347376H1 | g1432173 | 15 | −4 | gb105eukp | copB; CopB | |
| 700346947H1 | g1707867 | 83 | −32 | gb105pln | Z. mays mRNA for 40S ribosomal subunit protein S21. | |
| 700346276H1 | g1345132 | 14 | −10 | gb105eukp | ERECTA; ERECTA | |
| 700346909H1 | g218130 | 31 | −29 | gb105pln | Rice mRNA for Ribosomal protein S15. | |
| 700344119H1 | g600768 | 49 | −10 | gb105pln | Oryza sativa cyclophilin 2 (Cyp2) mRNA, complete cds. | |
| 700346123H1 | g2244825 | 35 | −3 | gb105eukp | light induced protein homolog | |
| 700345295H1 | g2511540 | 18 | 9 | gb105pln | Oryza sativa DNA-binding protein GBP16 (Rgbp16) mRNA, complete cds. | |
| 700344887H1 | g2444419 | 28 | −6 | gb105pln | Glycine max ribosome-associated protein p40 mRNA, complete cds. | |
| 700343330H1 | g414340 | 33 | −20 | gb105pln | A. thaliana rd21A gene for thiol protease, complete cds. | |
| 700346309H1 | g1185553 | 32 | −49 | gb105pln | Zea mays glyceraldehyde-3-phosphate dehydrogenase (gpc2) gene, complete cds. | |
| 700343229H1 | g1498052 | 77 | −63 | gb105pln | Zea mays ribosomal protein S8 mRNA, complete cds. | |
| 700347294H1 | g293888 | 82 | −79 | gb105pln | Zea mays, glyceraldehyde-3-phosphate dehydrogenase mRNA, 3′ end (clone GAPC2). | |
| 700345881H1 | g2827618 | 24 | −1 | gb105pln | Arabidopsis thaliana DNA chromosome 4, BAC clone F10N7 (ESSAII project). | |
| 700346714H1 | g1408471 | 13 | 5 | gb105eukp | ADF1; actin depolymerizing factor 1 | |
| 700344384H1 | g2344870 | 26 | −23 | gb105pln | Phaseolus vulgaris mRNA for purple acid phosphatase precursor. | |
| 700343960H1 | g1654186 | 23 | −2 | gb105allp | CTP synthetase homolog | |
| 700343223H1 | g1675393 | 24 | −35 | gb105pln | Oryza sativa class III ADH enzyme (AdhIII) gene, complete cds. | |
| 700343206H1 | g218142 | 20 | 4 | gb105pln | Rice mRNA for aspartic proteinase, complete cds. | |
| 700342524H1 | g170455 | 13 | 3 | gb105pln | Tomato heat shock cognate protein 80 gene, 3′ end. | |
| 700345433H1 | g22324 | 41 | −18 | gb105pln | Z. mays mRNA for H2B histone (clone cH2B221). | |
| 700344107H1 | g2326873 | 20 | −1 | gb105pln | S. tuberosum mRNA for plastidic ATP/ADP-transporter. | |
| 700343086H1 | g804949 | 33 | −26 | gb105pln | A. thaliana mRNA for cysteine synthase. | |
| 700380167H1 | g609289 | 62 | 13 | gb105pln | Z. mays cultivar (LG11) ROA mRNA for replication origin activator protein. | |
| 700346785H1 | g1749575 | 23 | −7 | gb105pln | Fission Yeast mRNA, complete cds. | |
| 700346541H1 | g488576 | 29 | −13 | gb105pln | Medicago sativa Regen S clone pH3c131 histone H3.2 mRNA, complete cds. | |
| 700379661H1 | g1370177 | 23 | −2 | gb105pln | L. japonicus mRNA for small GTP-binding protein, RAB5A. | |
| 700342505H1 | g2150129 | 36 | −22 | gb105pln | Arabidopsis thaliana cytoplasmic ribosomal protein S15a mRNA, complete cds. | |
| 700347201H1 | g1293686 | 7 | 8 | gb105allp | transcription factor C1 (HCF) | |
| 700344001H1 | g473604 | 37 | 12 | gb105pln | Zea mays W-22 histone H2B mRNA, complete cds. | |
| 700346867H1 | g1777706 | 67 | −74 | gb105pln | Zea mays 18S ribosomal RNA gene, partial sequence. | |
| 700380083H1 | g166605 | 42 | −4 | gb105pln | Arabidopsis thaliana anthranilate synthase alpha subunit gene, complete cds. | |
| 700379608H1 | g168500 | 46 | −45 | gb105pln | Maize (Zea mays) histone H4 gene (H4C14), complete cds. | |
| 700342985H1 | g886400 | 49 | −30 | gb105pln | Oryza sativa MADS-box protein (MADS2) mRNA, complete cds. | |
| 700342282H1 | g285951 | 20 | −1 | gb105allp | KIAA0107 | |
| 700345348H1 | g312180 | 32 | −52 | gb105pln | Z. mays GapC4 gene. | |
| 700343434H1 | g1255963 | 9 | −8 | gb105eukp | ORF N0852 | |
| 700342895H1 | g2341023 | 45 | −1 | gb105pln | Sequence of BAC F19P19 from Arabidopsis thaliana chromosome 1, complete sequence. | |
| 700380518H1 | g1906827 | 27 | −14 | gb105pln | A. thaliana hsp81.4 gene. | |
| 700380151H1 | g170772 | 35 | −51 | gb105pln | Triticum aestivum S-adenosyl-L-homocysteine hydrolase (SH6.2) mRNA, complete cds. | |
| 700346436H1 | g535515 | 36 | 5 | gb105allp | homolog of yeast mutL gene | |
| 700347018H1 | g21227 | 18 | 2 | gb105pln | Spinach mRNA for chloroplast 37 kD inner envelope membrane protein. | |
| 700345146H1 | g1147585 | 33 | 7 | gb105eukp | LysRS; ATP + L-LYSYNE + tRNA(LYS = AMP + PYROPHOSPHATE + L- | |
| | | | | | LYSYL-tRNA(LYS); Lysyl-tRNA synthetase; EC 6.1.1.6 | |
| 700343380H1 | g1052973 | 25 | −8 | gb105eukp | fructokinase; EC 2.7.1.4 | |
| 700342638H1 | g313759 | 45 | −31 | gb105pln | Z. mays hsp 70-1 gene for heat shock protein 70. | |
| 700347236H1 | g475212 | 12 | 15 | gb105pln | N. tabacum eIF-4A10 mRNA. | |
| 700343049H1 | g576506 | 14 | 5 | gb105pln | Pisum sativum outer membrane protein (IAP75) mRNA, complete cds. | |
| 700380712H1 | g473604 | 38 | −38 | gb105pln | Zea mays W-22 histone H2B mRNA, complete cds. | |
| 700342353H1 | g1710361 | 10 | −0 | gb105eukp | uaf-1; splicing factor U2AF65 | |
| 700342311H1 | g915193 | 45 | −38 | gb105pln | Chlamydomonas reinhardtii mRNA for actin, complete cds. | |
| 700347001H1 | g20255 | 64 | −54 | gb105pln | O. sativa gene for heat shock protein 82 HSP82. | |
| 700346968H1 | g217910 | 25 | −40 | gb105pln | Carrot mRNA for nuclear antigen 21D7. | |
| 700345522H1 | g398917 | 27 | −7 | gb105pln | B. napus cold induced protein (BnC24A) mRNA. | |
| 700344278H1 | g1574937 | 30 | −54 | gb105pln | Zea mays superoxide dismutase 4 (sod4) gene, partial cds. | |
| 700379967H1 | g21800 | 57 | −50 | gb105pln | T. aestivum L mRNA for histone H2B. | |
| 700346388H1 | g312178 | 17 | 10 | gb105pln | Z. mays GapC2 gene. | |
| 700343360H1 | g2288979 | 22 | −19 | gb105pln | Arabidopsis thaliana chromosome II BAC T01O24 genomic sequence, complete | |
| | | | | | sequence. | |
| 700380763H1 | g452164 | 30 | −22 | gb105pln | Cucumber mRNA for monodehydroascorbate reductase, complete cds. | |
| 700343316H1 | g525241 | 47 | −11 | gb105allp | glutaminyl-peptide cyclotransferase | |
| 700344931H1 | g169792 | 56 | −43 | gb105pln | Rice histone 3 gene, complete cds. | |
| 700342356H1 | g188565 | 82 | −22 | gb105allp | mitochondrial hinge protein precursor | |
| 700380121H1 | g577818 | 18 | −10 | gb105pln | Z. mays gene for H2B histone (gH2B4). | |
| 700379965H1 | g736271 | 65 | −33 | gb105pln | O. sativa hsp70 gene for heat shock protein 70. | |
| 700347125H1 | g623589 | 47 | −39 | gb105pln | Nicotiana tabacum SR1 Nt-rab7b mRNA, complete cds. | |
| 700347070H1 | g1061039 | 26 | −12 | gb105pln | A. thaliana mRNA for sterol-C-methyltransferase. | |
| 700346827H1 | g16876 | 39 | 5 | gb105allp | vacuolar H+-pyrophosphatase | |
| 700342321H1 | g862933 | 25 | −11 | gb105allp | protein kinase C inhitibor-I | |
| 700343446H1 | g2760165 | 22 | −10 | gb105pln | Arabidopsis thaliana genomic DNA, chromosome 5, P1 clone: MAC9, complete | |
| | | | | | sequence. | |
| 700342961H1 | g495697 | 20 | −8 | gb105eukp | F54E7.2 | |
| 700345629H1 | g18329 | 22 | −0 | gb105pln | D. caryophyllus CARSR8 mRNA for glutathione s-transferase. | |
| 700344065H1 | g1235678 | 46 | −4 | gb105allp | cystatin B | |
| 700345631H1 | g19654 | 15 | 4 | gb105eukp | protein disulfide isomerase | |
| 700345158H1 | g1255448 | 13 | 7 | gb105allp | mitogen-activated protein kinase | |
| 700344393H1 | g1049306 | 36 | −16 | gb105pln | Arabidopsis thaliana actin-2 mRNA, complete cds. | |
| 700344889H1 | g170107 | 20 | 1 | gb105allp | chaperonin 10 | |
| 700342213H1 | g22322 | 16 | −8 | gb105pln | Z. mays mRNA for H2B histone (cone cH2B214). | |
| 700347311H1 | g4392 | 13 | 3 | gb105eukp | RPL37A; ribosomal protein L37a | |
| 700345958H1 | g22483 | 28 | −14 | gb105pln | Z. mays RNA for superoxide dismutase Sod4. | |
| 700343843H1 | g1066931 | 22 | −5 | gb105eukp | F10B5.7 | |
| 700346666H1 | g393400 | 80 | −66 | gb105pln | Z. mays mRNA for alpha-tubulin. | |
| 700343263H1 | g520935 | 63 | −57 | gb105pln | H. vulgare mRNA for gamma-TIP-like protein. | |
| 700380756H1 | g22149 | 24 | 10 | gb105pln | Z. mays mRNA for alpha-tubulin 3. | |
| 700380283H1 | g168505 | 58 | −47 | gb105pln | Zea mays histone H3 gene, complete cds. | |
| 700380217H1 | g170433 | 17 | 8 | gb105allp | ATP-dependent protease (CD4A) | |
| 700346715H1 | g22747 | 17 | 3 | gb105pln | L. longiflorum mRNA for actin depolymerizing factor. | |
| 700346229H1 | g410487 | 41 | −32 | gb105pln | L. esculentum DAHP synthase 2 precursor. | |
| 700345214H1 | g506471 | 25 | −10 | gb105eukp | unnamed protein product | |
| 700281886H2 | g22657 | 24 | −7 | gb105pln | A. thaliana mRNA for ubiquitin-conjugating enzyme. | |
| 700344526H1 | g555616 | 12 | −0 | gb105eukp | cim1; cytokinin induced message | |
| 700342935H1 | g1015814 | 4 | 8 | gb105eukp | ORF YJR105w | |
| 700346242H1 | g167961 | 27 | −5 | gb105pln | D. caryophyllus S-adenosylmethionine synthetase (CARSAM2) mRNA, complete cds. | |
| 700380662H1 | g2828278 | 27 | −32 | gb105pln | Arabidopsis thaliana DNA chromosome 4, BAC clone T18B16 (ESSAII project). | |
| 700342712H1 | g313147 | 40 | −55 | gb105pln | A. medicago MSK-3 mRNA for protein kinase. | |
| 700347009H1 | g166682 | 15 | 5 | gb105eukp | CTR1; protein kinase | |
| 700342809H1 | g2808637 | 46 | −36 | gb105pln | Daucus carota mRNA for Rab8-like small GTP-binding protein. | |
| 700344865H1 | g20412 | 43 | −32 | gb105pln | P. amygdalus mRNA for alpha-tubulin. | |
| 700342220H1 | g1129085 | 40 | −32 | gb105pln | Wheat mRNA for protein H2A, complete cds, clone wcH2A-9. | |
| 700342364H1 | g218082 | 21 | 0 | gb105pln | Rice mRNA for initiation factor EIF-4D (225 gene), partial sequence. | |
| 700342313H1 | g2651294 | 15 | −3 | gb105pln | Arabidopsis thaliana chromosome IIBAC T2P4 genomic sequence, complete sequence. | |
| 700343167H1 | g633028 | 23 | −5 | gb105eukp | protein phosphatase 2C | |
| 700281992H2 | g575730 | 22 | −39 | gb105pln | Z. mays mRNA for transmembrane protein. | |
| 700342761H1 | g2149639 | 48 | −37 | gb105pln | Arabidopsis thialiana Argonaute protein (AGO1) mRNA, complete cds. | |
| 700342350H1 | g1402918 | 12 | 6 | gb105allp | peroxidase | |
| 700379829H1 | g166857 | 46 | −40 | gb105pln | Arabidopsis thaliana cytoplasmic robosomal protein mRNA, complete cds. | |
| 700344191H1 | g872116 | 11 | 5 | gb105allp | sti (stress inducible protein) | |
| 700344424H1 | g602757 | 22 | −10 | gb105pln | Lycopersicon esculentum clp-like energy-dependent protease mRNA, complete cds. | |
| 700343484H1 | g2501765 | 32 | −12 | gb105pln | Glycine max calmodulin-like domain protein kinase isoenzyme gamma mRNA, | |
| | | | | | complete cds. | |
| 700346954H1 | g19495 | 14 | −2 | gb105pln | L. perenne RNA for Lol pI gene. | |
| 700380563H1 | g2586150 | 16 | −17 | gb105pln | Musa acuminata ripening-associated protein mRNA, partial cds. | |
| 700346560H1 | g516328 | 12 | −3 | gb105eukp | Su(var)3-9; Su(var)3-9 | |
| 700345573H1 | g563334 | 18 | 6 | gb105pln | B. napus (Naehan) bgb1 mRNA for guanine nucleotide regulatory protein. | |
| 700345169H1 | g20321 | 60 | −49 | gb105pln | Oryza sativa RAc1 mRNA for actin. | |
| 700344283H1 | g21832 | 61 | −48 | gb105pln | Wheat mRNA for chloroplast phosphoplycerate kinase (EC 2.7.2.3). | |
| 700343802H1 | g2821957 | 32 | −19 | gb105eukp | spermidine synthase 2; EC 2.5.1.16 | |
| 700343696H1 | g296064 | 16 | 5 | gb105allp | RING1 gene product | |
| 700379660H1 | g20000 | 26 | −16 | gb105pln | N. tabacum RL2 mRNA for 60S ribosomal protein L2. | |
| 700346947H1 | g1419371 | 23 | 17 | gb105pln | Z. mays mRNA for 40S subunit ribosomal protein S21. | |
| 700346865H1 | g2351061 | 17 | −4 | gb105pln | Arabidopsis thaliana genomic DNA, chromosome 5, P1 clone: MAF19. | |
| 700346722H1 | g2160155 | 14 | 8 | gb105pln | Sequence of BAC F21M12 from Arabidopsis thaliana chromosome 1, complete | |
| | | | | | sequence. | |
| 700346243H1 | g31417 | 25 | −9 | gb105allp | fibulin-1 B | |
| 700379656H1 | g22292 | 57 | −55 | gb105pln | Z. mays for glycine-rich protein. | |
| 700346979H1 | g409074 | 18 | 2 | gb105allp | HBp15/L22 | |
| 700343049H1 | g633607 | 21 | −17 | gb105eukp | chloroplastic outer envelope membrane protein (OEP75) | |
| 700347190H1 | g2326343 | 21 | −5 | gb105eukp | g14587-5 | |
| 700380401H1 | g2773051 | 29 | −15 | gb105pln | Spinacia oleracea heat shock 70 protein (HSC70-11) gene, nuclear gene encoding | |
| | | | | | mitochondrial protein, complete cds. | |
| 700345257H1 | g551251 | 13 | 6 | gb105allp | ribosomal protein S7 | |
| 700345692H1 | g498772 | 47 | −68 | gb105pln | Z. mays (cv DH5xDH7) hsp70-4 mRNA for heat shock protein 70. | |
| 700380574H1 | g2696803 | 39 | −59 | gb105pln | Oryza sativa mRNA for water channel protein, complete cds. | |
| 700346718H1 | g780371 | 58 | −53 | gb105pln | Oryza sativa enolase mRNA, complete cds. | |
| 700345347H1 | g1653442 | 12 | 4 | gb105allp | hypothetical protein | |
| 700345524H1 | g17935 | 39 | −27 | gb105pln | B. vulgaris mRNA for salt-inducible betaine aldehyde dehydrogenase. | |
| 700344323H1 | g22322 | 20 | 3 | gb105pln | Z. mays mRNA for H2B histone (clone cH2B214). | |
| 700342578H1 | g1524382 | 76 | −83 | gb105pln | Z. mays mRNA for EPSP-synthase. | |
| 700380547H1 | g2668737 | 74 | −82 | gb105pln | Z. mays translation initiation factor 5A (TIF5A) mRNA, complete cds. | |
| 700346811H1 | g349054 | 45 | −29 | gb105pln | Solanum tuberosum ubiquitin/ribosomal fusion protein (ubi3) gene, complete cds. | |
| 700346463H1 | g736271 | 41 | −28 | gb105pln | O. sativa hsp70 gene for heat shock protein 70. | |
| 700347294H1 | g1185553 | 34 | −72 | gb105pln | Zea mays glyceraldehyde-3-phosphate dehydrogenase (gpc2) gene, complete cds. | |
| 700346463H1 | g1181672 | 48 | −39 | gb105pln | Sorghum bicolor heat shock protein 70 cognate (hsc70) mRNA, partial cds. | |
| 700344950H1 | g1550813 | 26 | −42 | gb105pln | Z. mays mRNA for acidic ribosomal protein P0. | |
| 700342901H1 | g1345483 | 37 | −0 | gb105allp | Elongation factor 1 beta | |
| 700380244H1 | g625147 | 95 | −86 | gb105pln | Zea mays protein disulfide isomerase (pdi) mRNA, complete cds. | |
| 700379817H1 | g168486 | 34 | −7 | gb105pln | Maize glutathione S-transferase gene (GST-I), exon 1. | |
| 700345549H1 | g22149 | 47 | −35 | gb105pln | Z. mays mRNA for alpha-tubulin 3. | |
| 700347170H1 | g21215 | 24 | −34 | gb105pln | S. guttatum aox1 gene for salicylic acid-inducible alternative oxidase. | |
| 700345744H1 | g168502 | 79 | −79 | gb105pln | Maize (Zea mays) histone H4 gene (H4C7), complete cds. | |
| 700347091H1 | g498741 | 42 | −26 | gb105pln | H. vulgare (pMaW25) mRNA for beta-ketoacyl-ACP synthase. | |
| 700346413H1 | g1399264 | 13 | −4 | gb105pln | Arabidopsis thaliana calmodulin-domain protein kinase CDPK isoform 9 (CPK9) | |
| | | | | | mRNA, complete cds. | |
| 700346251H1 | g2815519 | 17 | −8 | gb105pln | Arabidopsis thaliana BAC T5J8 from chromosome IV, top arm, complete sequence. | |
| 700343054H1 | g458972 | 5 | 7 | gb105eukp | F37C12.4 | |
| 700343219H1 | g551482 | 16 | −12 | gb105pln | Zea mays ABA- and ripening-inducible-like protein mRNA, complete cds. | |
| 700342955H1 | g2463334 | 34 | −41 | gb105pln | Oryza sativa mRNA for ribosomal protein S4. | |
| 700342275H1 | g2789659 | 35 | −24 | gb105pln | Arabidopsis thaliana p105 mRNA, complete cds. | |
| 700379889H1 | g1814402 | 60 | −21 | gb105pln | Mesembryanthemum crystallinum methionine synthase (MetE) mRNA, complete cds. | |
| 700347150H1 | g2828295 | 9 | 7 | gb105eukp | T18B16.170; putative protein | |
| 700281877H2 | g1431621 | 45 | −25 | gb105pln | T. repens mRNA for protein kinase. | |
| 700344254H1 | g2632345 | 9 | −7 | gb105allp | dihydroneopterin aldolase | |
| 700343922H1 | g454913 | 43 | −34 | gb105pln | A. porrum LDJ2 mRNA. | |
| 700345395H1 | g2570119 | 17 | 10 | gb105pln | S. latifolia mRNA, clone CCLS 86.1. | |
| 700343844H1 | g312178 | 35 | −72 | gb105pln | Z. mays GapC2 gene. | |
| 700346147H1 | g463856 | 26 | −4 | gb105pln | Chlamydomonas reinhardtii 21gr ribosomal protein S14 (CRY1) gene, complete cds. | |
| 700343541H1 | g1762984 | 21 | −1 | gb105allp | WD40-repeat type I transmembrane protein A72.5 | |
| 700344914H1 | g303852 | 63 | −56 | gb105pln | Rice mRNA for ribosomal protein L3, complete cds. | |
| 700379653H1 | g168292 | 14 | 2 | gb105eukp | ORF1 | |
| 700346726H1 | g13667 | 27 | −6 | gb105eukp | cytochrome oxidase subunit 1 (AA 1-517) | |
| 700379628H1 | g287834 | 40 | −24 | gb105pln | Z. mays yptm2 cDNA. | |
| 700346745H1 | g577612 | 14 | 6 | gb105eukp | CRT1; calcium sequestration; calcium-binding protein; calreticulin | |
| 700344950H1 | g415316 | 14 | −20 | gb105pln | Rice mRNA for acidic ribosomal protein P0, complete cds. | |
| 700343116H1 | g587074 | 12 | 6 | gb105allp | putative IEF | |
| 700346441H1 | g2282034 | 9 | 1 | gb105allp | p41-Arc | |
| 700345275H1 | g393400 | 46 | −40 | gb105pln | Z. mays mRNA for alpha-tubulin. | |
| 700343558H1 | g1389639 | 28 | −29 | gb105pln | Pea mRNA for PNDKN1, complete cds. | |
| 700346780H1 | g736271 | 53 | −60 | gb105pln | O. sativa hsp70 gene for heat shock protein 70. | |
| 700345518H1 | g2668739 | 25 | −2 | gb105pln | Zea mays translation initiation factor GOS2 (TIF) mRNA, complete cds. | |
| 700344056H1 | g1408277 | 48 | −14 | gb105allp | proline rich protein | |
| 700343762H1 | g642910 | 18 | −7 | gb105pln | Lycopersicon esculentum (clone GFPGSAAM) glutamate 1-semialdehyde 2,1- | |
| | | | | | aminomutase mRNA, complete cds. | |
| 700346361H1 | g432367 | 40 | −27 | gb105pln | Rice mRNA for elongation factor 1 beta, complete cds. | |
| 700344179H1 | g2347208 | 12 | −2 | gb105eukp | T09D09.12; APG protein isolog | |
| 700345355H1 | g1041709 | 23 | −18 | gb105pln | Oryza sativa expansin Os-EXP2 (Os-EXP2) mRNA, complete cds. | |
| 700343523H1 | g415634 | 22 | −5 | gb105eukp | cloned by ability to arrest the cell cycle when expressed in the fission yeast | |
| | | | | | Schizosaccharomyces pombe; homolog of human 40S ribosomal protein S25, | |
| | | | | | Dictyostelium 40S ribosomal protein S31 and Saccharomyces 40S ribosomal protein S31 | |
| 700342960H1 | g2275016 | 55 | −41 | gb105allp | elastase | |
| 700346257H1 | g218178 | 22 | −25 | gb105pln | Rice OSA1 gene for H+-ATPase, complete cds. | |
| 700347258H1 | g1620908 | 22 | −2 | gb105pln | Carrot mRNA for DcARF1, complete cds. | |
| 700345538H1 | g474007 | 38 | −7 | gb105pln | Rice mRNA, partial homologous to ribosomal protein S12 gene. | |
| 700345474H1 | g218132 | 52 | −39 | gb105pln | Rice mRNA for Heat shock protein 83. | |
| 700345080H1 | g2668745 | 76 | −92 | gb105pln | Zea mays inorganic pyrophosphatase (IPP) mRNA, complete cds. | |
| 700346547H1 | g1841307 | 16 | 8 | gb105pln | Fission Yeast mRNA for ribosomal protein S16 homolog, partial cds. | |
| 700345104H1 | g536895 | 65 | −58 | gb105pln | Wheat mRNA for protein H2A, complete cds, clone wcH2A-10. | |
| 700344194H1 | g871985 | 59 | −48 | gb105pln | A. sativa Aspk11 mRNA. | |
| 700345416H1 | g19342 | 33 | −22 | gb105pln | L. esculentum mRNA for ribosomal protein L2. | |
| 700343407H1 | g2583148 | 24 | −7 | gb104eukp | pfk; 6-phospho-1-fructokinase; EC 2.7.1.11 | |
| 700343834H1 | g1100224 | 36 | −20 | gb105pln | Pinus sylvestris chloroplast NAD+-dependent glyceraldehyde-3-phosphate | |
| | | | | | dehydrogenase mRNA, complete cds. | |
| 700380638H1 | g1675393 | 24 | −25 | gb105pln | Oryza sativa class III ADH enzyme (AdhIII) gene, complete cds. | |
| 700379870H1 | g1911165 | 25 | −13 | gb105pln | S. tuberosum mRNA for soluble-starch-synthase. | |
| 700346455H1 | g22312 | 100 | −14 | gb105pln | Maize ABA-inducible gene for glycine-rich protein (ABA = abscisic acid). | |
| 700345055H1 | g857573 | 68 | −46 | gb105pln | Oryza sativa vacuolar H+-ATPase (vatp-P1) mRNA, complete cds. | |
| 700344508H1 | g1149569 | 9 | −1 | gb105eukp | athb-8; HD-zip | |
| 700346544H1 | g498906 | 34 | −12 | gb105eukp | RPL27-5; ribosomal protein L27 homolog | |
| 700345875H1 | g571474 | 51 | −36 | gb105pln | Chlamydomonas reinhardtii histone H3 (ch3-III), histone H4 (ch4-III), histone H2B | |
| | | | | | (ch2b-III) and histone H2A (ch2a-III) genes, complete cds. | |
| 700345535H1 | g2352427 | 7 | 6 | gb105allp | peroxisomal Ca-dependent solute carrier | |
| 700343012H1 | g473604 | 83 | −70 | gb105pln | Zea mays W-22 histone H2B mRNA, complete cds. | |
| 700379614H1 | g2511530 | 60 | −50 | gb105pln | Eleusine indica alpha tubulin 1 (TUA1) mRNA, complete cds. | |
| 700347206H1 | g470126 | 35 | −15 | gb105pln | N. tabacum (cv. Samsun NN) L19 mRNA for ribosomal protein L19. | |
| 700346735H1 | g1136119 | 56 | −56 | gb105pln | O. sativa mRNA for alpha-tubulin (clone OSTA-111). | |
| 700343934H1 | g473986 | 15 | −13 | gb105pln | Rice mRNA, partial homologous to histone H2B gene. | |
| 700345245H1 | g2414648 | 20 | −3 | gb105eukp | rp17-b; 60s ribosomal protein | |
| 700346477H1 | g2244792 | 12 | 7 | gb105allp | ankyrin homolog | |
| 700345308H1 | g2832636 | 13 | 7 | gb105eukp | hypothetical protein | |
| 700345704H1 | g395071 | 40 | −42 | gb105pln | V. faba guanine nucleotide regulatory protein mRNA, complete cds. | |
| 700344319H1 | g2623247 | 26 | −12 | gb105pln | Zea mays SU1 isomylase (sugaryl) gene, complete cds. | |
| 700345362H1 | g557681 | 52 | −32 | gb105pln | Z. mays mRNA for ribosomal protein S28. | |
| 700344996H1 | g2829865 | 46 | −23 | gb105eukp | F3I6.4 | |
| 700343840H1 | g2511530 | 79 | −67 | gb105pln | Eleusine indica alpha tubulin 1 (TUA1) mRNA, complete cds. | |
| 700342780H1 | g620050 | 16 | −11 | gb105eukp | T15H9.1 | |
| 700345826H1 | g902524 | 72 | −75 | gb105pln | Zea mays clone MubG10 ubiquitin fusion protein gene, complete cds. | |
| 700345562H1 | g493588 | 61 | −54 | gb105pln | Hordeum vulgare disulfide isomerase (PDI) mRNA, complete cds. | |
| 700379651H1 | g474003 | 27 | 6 | gb105pln | Rice mRNA, partial homologous to ribosomal protein rp21c gene. | |
| 700344871H1 | g1526426 | 9 | 7 | gb105allp | proteasome subunit p42 | |
| 700344496H1 | g458426 | 9 | 7 | gb105eukp | dihydrolipoamide acetyltransferase | |
| 700281806H2 | g624220 | 64 | −23 | gb105pln | Oryza sativa signal recognition particle 19 kDa protein subunit SRP19 mRNA, | |
| | | | | | complete cds. | |
| 700346719H1 | g2668739 | 95 | −90 | gb105pln | Zea mays translation initiation factor GOS2 (TIF) mRNA, complete cds. | |
| 700345120H1 | g2668749 | 45 | −33 | gb105pln | Zea mays ribosomal protein L30 (rpl30) mRNA, complete cds. | |
| 700343484H1 | g836943 | 19 | −11 | gb105pln | Arabidopsis thaliana calcium-dependent kinase (CDPK6) gene, complete cds. | |
| 700380557H1 | g416252 | 29 | −16 | gb105pln | Rice mRNA for 3-deoxy-D-arabino-heptulosonate 7-phosphate synthase, partial | |
| | | | | | sequence. | |
| 700344155H1 | g498906 | 31 | −29 | gb105eukp | RPL27-5; ribosomal protein L27 homolog | |
| 700346835H1 | g1100222 | 35 | −23 | gb105pln | Pinus sylvestris chloroplast NAD+-dependent glyceraldehyde-3-phosphate | |
| | | | | | dehydrogenase mRNA, complete cds. | |
| 700346790H1 | g509264 | 23 | −13 | gb105pln | B. napus mRNA for acyl-CoA binding protein. | |
| 700343740H1 | g425274 | 7 | 5 | gb105eukp | synaptotagmin, p65 | |
| 700343065H1 | g248338 | 39 | −42 | gb105pln | polyubiquitin [maize, Genomic, 3439 nt]. | |
| 700343038H1 | g904027 | 44 | −33 | gb105pln | Triticum aestivum nucleosome assembly protein I mRNA, partial cds. | |
| 700345539H1 | g22118 | 13 | 16 | gb105pln | Z. mays DNA for Adh1-Cm allele. | |
| 700344895H1 | g387112 | 35 | −26 | gb105allp | 34 kDa beta-galactoside-binding lectin (L-34) | |
| 700281810H2 | g2529253 | 25 | −0 | gb105eukp | rpb12; RNA polymarase II subunit Rpb12; EC 2.7.7.6 | |
| 700342830H1 | g2668737 | 52 | −49 | gb105pln | Zea mays translation initiation factor 5A (TIF5A) mRNA, complete cds. | |
| 700380650H1 | g168500 | 57 | −45 | gb105pln | Maize (Zea mays) histone H4 gene (H4C14), complete cds. | |
| 700346888H1 | g899607 | 68 | −36 | gb105pln | Zea mays polyubiquitin (MubC5) mRNA, complete cds. | |
| 700343322H1 | g1657763 | 13 | 10 | gb105pln | Zea mays retrotransposon Ji-3 5′ LTR and primer binding site DNA sequence. | |
| 700343295H1 | g2760839 | 15 | −2 | gb105eukp | F18A8.10; putative receptor kinase | |
| 700380070H1 | g1435156 | 50 | −6 | gb105pln | L. esculentum mRNA for histone H3 variant H3.3. | |
| 700343623H1 | g1322844 | 22 | −6 | gb105eukp | SPT16 | |
| 700342343H1 | g602398 | 20 | −2 | gb105eukp | SPF1; P-type ATPase | |
| 700343717H1 | g600390 | 17 | −1 | gb105pln | A. thaliana UbcAT4b mRNA for ubiquitin conjugating enzyme E2. | |
| 700281969H2 | g2791884 | 18 | −3 | gb105pln | Arabidopsis thaliana JAB1 (JAB1) mRNA, complete cds. | |
| 700281967H1 | g514945 | 54 | −82 | gb105pln | Zea mays sucrose synthase (Sus1) mRNA, complete cds. | |
| 700347133H1 | g1668706 | 51 | 0 | gb105eukp | atran2 | |
| 700346930H1 | g2827559 | 8 | 8 | gb105eukp | T12H17.210; predicted protein | |
| 700343874H1 | g2827650 | 27 | −13 | gb105eukp | F18F4.60; potassium transporter-like protein | |
| 700344108H1 | g1206016 | 14 | 12 | gb105pln | Yeast (Schizosaccharomyces pombe) ribosomal protein L5 gene, complete cds. | |
| 700346968H1 | g1864002 | 23 | −35 | gb105pln | Nicotiana tabacum mRNA for 21D7, complete cds. | |
| 700346207H1 | g141900 | 8 | 6 | gb105allp | alcohol dehydrogenase (EC 1.1.1.1) | |
| 700281877H2 | g456508 | 47 | −27 | gb105pln | A. thaliana ASK-gamma mRNA. | |
| 700380172H1 | g2414643 | 28 | −15 | gb105pln | Yeast (Saccharomyces pombe) chromosome I cosmid c3H5. | |
| 700346992H1 | g2443472 | 41 | −19 | gb105eukp | SF2; ASF/SF2 homolog | |
| 700346309H1 | g312178 | 32 | −52 | gb105pln | Z. mays GapC2 gene. | |
| 700345729H1 | g1272634 | 13 | 1 | gb105eukp | K07C5.4 | |
| 700344480H1 | g2564051 | 39 | −1 | gb105pln | Arabidopsis thaliana genomic DNA, chromosome 5, P1 clone: MWD9, complete | |
| | | | | | sequence. | |
| 700345085H1 | g1755008 | 76 | −66 | gb105pln | Triticum aestivum calmodulin TaCaM4-1 mRNA, complete cds. | |
| 700343811H1 | g2459531 | 27 | −14 | gb105eukp | Mf-FatB1; acyl-ACP thioesterase | |
| 700342716H1 | g2288886 | 24 | −9 | gb105pln | Arabidopsis thaliana mRNA for mevaolonate diphosphate decarboxylase. | |
| 700345974H1 | g2827001 | 63 | −12 | gb105pln | Triticum aestivum 70 kDa heat shock protein (TaHSP70d) mRNA, complete cds. | |
| 700343574H1 | g2267005 | 35 | −19 | gb105pln | Oryza sativa endosperm lumenal binding protein (BiP) mRNA, complete cds. | |
| 700343059H1 | g577611 | 70 | −83 | gb105pln | Zea mays CRT1 gene for calcium-binding protein. | |
| 700342287H1 | g459017 | 6 | −4 | gb105eukp | atp6; subunit 6 of the ATPase complex | |
| 700342532H1 | g1438971 | 6 | 5 | gb105allp | potassium channel Kv8.1 | |
| 700347070H1 | g2246455 | 19 | −1 | gb105pln | Arabidopsis thaliana S-adenosyl-methionine-sterol-C-methyltransferase mRNA, | |
| | | | | | complete cds. | |
| 700343890H1 | g1845194 | 41 | −59 | gb105pln | Z. mays mRNA for HMGc1 protein. | |
| 700343209H1 | g1770036 | 15 | 1 | gb105allp | hypothetical protein | |
| 700380711H1 | g2827081 | 39 | −37 | gb105pln | Medicago sativa cytosolic malate dehydrogenase (cmdh) mRNA, complete cds. | |
| 700379992H1 | g556409 | 12 | 4 | gb105eukp | RITA-1; transcriptional activator protein | |
| 700344220H1 | g391602 | 34 | −34 | gb105pln | Arabidopsis thaliana mRNA for casein kinase II catalystic subunit. | |
| 700346872H1 | g1245870 | 15 | 3 | gb105allp | replication factors MCM = dpa [Drosophila, Peptide, 866 aa] | |
| 700346067H1 | g349405 | 14 | 17 | gb105pln | Brassica napus (clone BnNa) DNA sequence. | |
| 700344372H1 | g285999 | 18 | 3 | gb105allp | KIAA0017 | |
| 700346883H1 | g2262163 | 34 | −7 | gb105eukp | F5J6.8; hypothetical protein similar to F5J6.10 | |
| 700344784H1 | g1248203 | 14 | 7 | gb105allp | Synthetic mature subtilisin 309 gene | |
| 700344201H1 | g415316 | 22 | −23 | gb105pln | Rice mRNA for acidic ribosomal protein P0, complete cds. | |
| 700344836H1 | g2689242 | 27 | −5 | gb105pln | A. thaliana mRNA for ubiquitin conjugating enzyme UBC2. | |
| 700347390H1 | g2160160 | 44 | −25 | gb105eukp | F21M12.5 | |
| 700342828H1 | g2414643 | 23 | −4 | gb105pln | Yeast (Saccharomyces pombe) chromosome I cosmid c3H5. | |
| 700380444H1 | g643596 | 33 | −9 | gb105pln | Corn mRNA for cysteine proteinase, clone CCP, complete cds. | |
| 700343339H1 | g2736191 | 15 | 5 | gb105allp | DfrA | |
| 700342484H1 | g1360177 | 12 | 7 | gb105allp | ORF YLL011w | |
| 700380569H1 | g8040 | 25 | −0 | gb105eukp | histone H2A variant (AA 1-141) | |
| 700343166H1 | g2351577 | 19 | −2 | gb105pln | Prunus armeniaca adenylate kinase homolog mRNA, complete cds. | |
| 700344091H1 | g1132482 | 38 | −18 | gb105pln | Rice mRNA for ADP-ribosylation factor, complete cds. | |
| 700343229H1 | g168543 | 100 | −36 | gb105pln | Zea mays putative ribosomal protein S8 mRNA, partial cds. | |
| 700343924H1 | g459199 | 18 | 7 | gb105pln | Gossypium hirsutum vacuolar H+-ATPase subunit B mRNA, partial cds. | |
| 700343660H1 | g1009237 | 52 | −53 | gb105pln | Tupeia antarctica (Forst. f.) Chamb. and Schlecht. 18S ribosomal RNA gene, partial | |
| | | | | | sequence. | |
| 700342370H1 | g21796 | 58 | −47 | gb105pln | Wheat histone H3 gene. | |
| 700347302H1 | g2599103 | 7 | 15 | gb105pln | Dunaliella salina 60S ribosomal protein (DSRP1) mRNA, complete cds. | |
| 700343219H1 | g1401233 | 17 | 3 | gb105pln | Pinus taeda water deficit inducible protein LP3-3 (lp3-3) mRNA, partial cds. | |
| 700346088H1 | g1360388 | 6 | 7 | gb105eukp | ORF YLR051c | |
| 700343157H1 | g644492 | 80 | −79 | gb105pln | Corn elongation factor 1alpha gene, complete cds. | |
| 700342896H1 | g1469220 | 35 | −8 | gb105pln | B. oleracea mRNA (unknown). | |
| 700342446H1 | g1051257 | 59 | −48 | gb105pln | Hordeum vulgare vacuolar ATPase catalystic subunit mRNA, partial cds. | |
| 700345652H1 | g203604 | 59 | 2 | gb105allp | contiguous repeat polypeptide | |
| 700344609H1 | g1732515 | 9 | 5 | gb105allp | myosin heavy chain-like protein | |
| 700344191H1 | g2408032 | 11 | 6 | gb105allp | hypothetical protein | |
| 700342352H1 | g2353331 | 25 | −14 | gb105eukp | rpS19; ribosomal protein S19 | |
| 700344194H1 | g508307 | 30 | −17 | gb105pln | Arabidopsis thaliana protein-serine kinase mRNA, complete cds. | |
| 700344010H1 | g473604 | 9 | 14 | gb105pln | Zea mays W-22 histone H2B mRNA, complete cds. | |
| 700347001H1 | g1906827 | 54 | −44 | gb105pln | A. thaliana hsp81.4 gene. | |
| 700344191H1 | g1688076 | 10 | 7 | gb105allp | tetratricopeptide repeat protein | |
| 700342482H1 | g169589 | 13 | 4 | gb105eukp | U2B; U2snRNP-specific protein; spliceosomal protein | |
| 700347067H1 | g1772495 | 6 | 6 | gb105eukp | HMB-CoA synthase; hydroxymethylglutaryl-CoA synthase; EC 4.1.3.5 | |
| 700345565H1 | g1724101 | 58 | −46 | gb105pln | Mesembryanthemum crystallinum S-adenosyl-L-homocystein hydrolase mRNA, | |
| | | | | | complete cds. | |
| 700343332H1 | g603601 | 15 | −0 | gb105eukp | NTF2; Ntf2p: Nuclear Transport Factor 2 | |
| 700379666H1 | g168500 | 25 | −44 | gb105pln | Maize (Zea mays) histone H4 gene (H4C14), complete cds. | |
| 700347314H1 | g533251 | 26 | −19 | gb105pln | Zea mays (clone pSM8) sucrose synthase 2 (Sus1) gene, complete cds. | |
| 700343045H1 | g2190976 | 12 | 4 | gb105allp | macrophage migration inhibitory factor | |
| 700343386H1 | g486551 | 11 | 7 | gb105allp | ORF YKR076w | |
| 700281876H2 | g2414402 | 28 | −17 | gb105eukp | Y57G11C.15 | |
| 700380593H1 | g168639 | 41 | −74 | gb105pln | Zea mays autonomous transposable element En-1 mosaic protein gene, complete cds. | |
| 700343157H1 | g2282583 | 83 | −81 | gb105pln | Zea mays elongation factor 1-alpha (EF1-A) mRNA, complete cds. | |
| 700344005H1 | g736271 | 51 | −25 | gb105pln | O. sativa hsp70 gene for heat shock protein 70. | |
| 700347378H1 | g2668748 | 31 | −19 | gb105eukp | rp117; ribosomal protein L17 | |
| 700346242H1 | g450548 | 36 | −16 | gb105pln | O. sativa (pRSAM-1) gene for S-adenosyl methionine synthetase. | |
| 700379824H1 | g403228 | 19 | 7 | gb105allp | ribonucleotide reductase small subunit | |
| 700346865H1 | g474009 | 65 | −49 | gb105pln | Rice mRNA, partial homologus to ribosomal protein S19 gene. | |
| 700343526H1 | g2244881 | 15 | 3 | gb105eukp | PDR5-like ABC transporter | |
| 700344660H1 | g452712 | 40 | −6 | gb105eukp | beta-galactosidase; EC 3.2.1.23 | |
| 700342367H1 | g902585 | 44 | −64 | gb105pln | Zea mays clone MubG9 ubiquitin gene, complete cds. | |
| 700379726H1 | g496271 | 23 | −2 | gb105pln | Nicotiana tabacum small Ras-like GTP-binding protein (Ran-B1) mRNA, complete cds. | |
| 700343063H1 | g2245029 | 11 | 6 | gb105eukp | limonene cyclase homolog | |
| 700346629H1 | g218182 | 40 | −22 | gb105pln | Rice mRNA for oryzain beta (EC 3.4.22). | |
| 700344132H1 | g1786871 | 8 | 7 | gb105allp | f311; 100 pct identical to fragment YBEK_ECOLI SW: P41409 (123 aa) but contains | |
| | | | | | 188 additional C-ter residues | |
| 700344316H1 | g1420686 | 14 | −2 | gb105eukp | RPL18B | |
| 700345189H1 | g602301 | 13 | 14 | gb105pln | Arabidopsis thaliana diminuto (DIM) mRNA, complete cds. | |
| 700344065H1 | g291927 | 46 | −4 | gb105allp | cystatin B | |
| 700344170H1 | g16086 | 23 | −4 | gb105pln | A. porrum dnaJ mRNA for DNA J protein (partial). | |
| 700343466H1 | g17645 | 49 | −5 | gb105allp | RIBOSOMAL PROTEIN L35a | |
| 700342763H1 | g2689469 | 19 | 3 | gb105eukp | IAA22 | |
| 700346208H1 | g218334 | 34 | −32 | gb105pln | Triticum aestivum mRNA for O-acetylserine (thiol) lyase. | |
| 700343436H1 | g1872521 | 20 | −5 | gb1053eukp | LSD1; negative regulator of cell death; zinc-finger protein Lsd1 | |
| 700379728H1 | g387908 | 36 | −32 | gb105pln | Brassica rapa S-phase-specific (BIS289) mRNA, complete cds. | |
| 700346636H1 | g22324 | 33 | −23 | gb105pln | Z. mays mRNA for H2B histone (clone cH2B221). | |
| 700346165H1 | g1877024 | 68 | −5 | gb105pln | Rice mitochondrial DNA for ribosomal protein L2, ribosomal protein S19, | |
| | | | | | complete cds. | |
| 700343507H1 | g1755008 | 44 | −33 | gb105pln | Triticum aestivum calmodulin TaCaM4-1 mRNA, complete cds. | |
| 700343395H1 | g498741 | 39 | −22 | gb105pln | H. vulgare (pMaW25) mRNA for beta-ketoacyl-ACP synthase. | |
| 700343155H1 | g1263313 | 26 | −8 | gb105pln | Arabidopsis thaliana molybdenum cofactor biosyntheses enzyme (cnx1) mRNA, | |
| | | | | | complete cds. | |
| 700346272H1 | g2764992 | 13 | 3 | gb105allp | plasma membrane polypeptide | |
| 700343966H1 | g2245109 | 17 | −2 | gb105eukp | carboxyl-terminal proteinase homolog | |
| 700343424H1 | g2414643 | 37 | −27 | gb105pln | Yeast (Saccharomyces pombe) chromosome I cosmid c3H5. | |
| 700346313H1 | g57002 | 8 | 6 | gb105allp | pyrimidine binding protein 1 | |
| 700347305H1 | g1314086 | 7 | 6 | gb105eukp | YPR011C; unknown | |
| 700346506H1 | g1845196 | 27 | −24 | gb105pln | Z. mays mRNA for HMGc2 protein. | |
| 700380788H1 | g899607 | 59 | −49 | gb105pln | Zea mays polyubiquitin (MubC5) mRNA, complete cds. | |
| 700380186H1 | g303834 | 31 | −32 | gb105pln | Rice mRNA for 21kd polypeptide, complete cds. | |
| 700344337H1 | g1262279 | 19 | −6 | gb105eukp | MDL3; catalyzes dissociation of (R)-mandelonitrile to hydrogen cyanide and | |
| | | | | | benzaldehyde; (R)-(+)-mandelonitrile lyase isoform precursor; EC 4.1.2.10 | |
| 700346193H1 | g514945 | 43 | −7 | gb105pln | Zea mays sucrose synthase (Sus1) mRNA, complete cds. | |
| 700347163H1 | g2407800 | 16 | 7 | gb105eukp | H2Av; histone H2A.F/Z | |
| 700281828H2 | g902583 | 67 | −60 | gb105pln | Zea mays clone MubG1 ubiquitin gene, complete cds. | |
| 700342650H1 | g927571 | 95 | −51 | gb105pln | Z. mays mRNA for calreticulin precursor. | |
| 700343586H1 | g552530 | 14 | −10 | gb105eukp | cytochrome b | |
| 700344734H1 | g2431768 | 62 | −61 | gb105pln | Zea mays acidic ribosomal protein Pla (rppla) mRNA, complete cds. | |
| 700347107H1 | g218237 | 20 | −11 | gb105pln | Rice mRNA for ribosomal protein S22 (T47 gene), partial sequence. | |
| 700345888H1 | g2352492 | 16 | −4 | gb105eukp | TIR1; transport inhibitor response 1 | |
| 700343162H1 | g395156 | 27 | −11 | gb105pln | Yeast (Saccharomyces cerevisiae) PRP8 gene, complete CDS. | |
| 700346527H1 | g1276967 | 18 | −7 | gb105eukp | putative ribosomal protein | |
| 700343777H1 | g2814254 | 17 | 3 | gb105eukp | JC8.g | |
| 700347033H1 | g2302292 | 14 | 7 | gb105allp | unnamed protein product | |
| 700347003H1 | g1321660 | 32 | −32 | gb105pln | Rice mRNA for ascorbate peroxidase, complete cds. | |
| 700345785H1 | g577276 | 9 | 6 | gb105allp | ring canal protein | |
| 700380179H1 | g1322276 | 28 | −5 | gb105pln | Triticum aestivum histone H2A gene, complete cds. | |
| 700343151H1 | g1502354 | 25 | −10 | gb105pln | Yeast (Saccharomyces cerevisiae) genomic sequence from chromosome VII. | |
| 700379705H1 | g22447 | 13 | 14 | gb105pln | Zea mays ZMPMS2 gene for 19 kDa zein protein. | |
| 700343013H1 | g20163 | 43 | −32 | gb105pln | O. sativa Rr15 mRNA for 5S ribosomal RNA. | |
| 700380670H1 | g2282583 | 58 | −60 | gb105pln | Zea mays elongation factor 1-alpha (EF1-A) mRNA, complete cds. | |
| 700344814H1 | g2285928 | 31 | −13 | gb105eukp | signal transduction; receptor tyrosine kinase | |
| 700281833H2 | g2696803 | 21 | 6 | gb105pln | Oryza sativa mRNA for water channel protein, complete cds. | |
| 700281805H2 | g20255 | 56 | −50 | gb105pln | O. sativa gene for heat shock protein 82 HSP82. | |
| 700343702H1 | g1088350 | 8 | 5 | gb105allp | adenylate kinase | |
| 700342309H1 | g2408032 | 10 | 6 | gb105eukp | SPAC17G6.19c; hypothetical protein | |
| 700346667H1 | g1617270 | 17 | 3 | gb105eukp | acyl-CoA synthetase; EC 6.2.1.3 | |
| 700346210H1 | g1783230 | 12 | 8 | gb105allp | aminopeptidase | |
| 700345305H1 | g1244773 | 14 | 1 | gb105eukp | RPL37A; Rp137ap: 60S ribosomal protein L37a | |
| 700380279H1 | g1666172 | 45 | −39 | gb105pln | N. plumbaginifolia mRNA for BTF3-like transcription factor. | |
| 700346248H1 | g998429 | 36 | −17 | gb105pln | GRF1 = general regulartory factor [Zea mays, XL80, Genomic, 5348 nt]. | |
| 700347121H1 | g296203 | 61 | −60 | gb105pln | P. miliaceum mRNA for alanine aminotransferase. | |
| 700345756H1 | g1946354 | 24 | −26 | gb105pln | Arabidopsis thaliana chromosome II BAC T06B20 genomic sequence, complete | |
| | | | | | sequence. | |
| 700344139H1 | g790969 | 65 | −58 | gb105pln | Rice mRNA for aldolase C-1, complete cds. | |
| 700347084H1 | g577818 | 26 | −19 | gb104pln | Z. mays gene for H2B histone (gH2B4). | |
| 700343001H1 | g50113 | 39 | −16 | gb105eukp | POL3; catalytic subunit DNA polymerase delta | |
| 700346704H1 | g1903420 | 5 | 5 | gb105allp | p71 | |
| 700343950H1 | g167607 | 23 | 4 | gb105eukp | actin 7 | |
| 700346461H1 | g514945 | 83 | −68 | gb105pln | Zea mays sucrose synthase (Sus1) mRNA, complete cds. | |
| 700343017H1 | g1197576 | 40 | −26 | gb105pln | H. vulgare mRNA for serpin. | |
| 700342305H1 | g166820 | 34 | −17 | gb105pln | Arabidopsis thaliana protein phosphatase mRNA, complete cds. | |
| 700380696H1 | g780371 | 75 | −66 | gb105pln | Oryza sativa enolase mRNA, complete cds. | |
| 700344968H1 | g1165205 | 19 | 3 | gb105pln | Arabidopsis thaliana multiubiquitin chain binding protein (MBP1) mRNA, complete cds. | |
| 700343484H1 | g836939 | 34 | −17 | gb105pln | Arabidopsis thaliana calcium-dependent protein kinase (CDPK6) mRNA, complete cds. | |
| 700342512H1 | g2264306 | 37 | −27 | gb105pln | Arabidopsis thaliana genomic DNA, chromosome 5, P1 clone: MBK5, complete | |
| | | | | | sequence. | |
| 700342756H1 | g1049306 | 43 | −4 | gb105pln | Arabidopsis thaliana actin-2 mRNA, complete cds. | |
| 700345862H1 | g1617268 | 14 | −2 | gb105eukp | acyl CoA synthetase; EC 6.2.1.3 | |
| 700344066H1 | g2232174 | 24 | 8 | gb105allp | MLD | |
| 700343495H1 | g998429 | 30 | −42 | gb105pln | GRF1 = general regulatory factor [Zea mays, XL80, Genomic, 5348 nt]. | |
| 700345823H1 | g643596 | 90 | −71 | gb105pln | Corn mRNA for cysteine proteinase, clone CCP, complete cds. | |
| 700344675H1 | g189049 | 14 | 1 | gb105allp | NADH dehydrogenase (ubiquinone) | |
| 700343248H1 | g22192 | 31 | −5 | gb105pln | Z. mays B-I gene for B transcriptional activator. | |
| 700346389H1 | g1749440 | 12 | 7 | gb105eukp | similar to Saccharomyces cerevisiae unknown, EMBL Accession Number Z47815 | |
| 700344637H1 | g2323344 | 28 | −13 | gb105eukp | Aglu1; alpha-glucosidase 1; EC 3.2.1.20 | |
| 700379927H1 | g218241 | 21 | −36 | gb105pln | Rice mRNA for ribosomal protein L3 (T82 gene), partial sequence. | |
| 700379647H1 | g1089850 | 69 | −38 | gb105allp | DAP-3 gene product | |
| 700345537H1 | g171053 | 15 | −0 | gb105eukp | AMP deaminase (EC 3.5.4.6) | |
| 700344182H1 | g169022 | 48 | −38 | gb105pln | Pisum sativum chloroplast stromal 70 kDa heat shock protein (hsp70) mRNA, | |
| | | | | | complete cds. | |
| 700342944H1 | g1360322 | 16 | −4 | gb105eukp | ORF YLR019w | |
| 700346079H1 | g439585 | 33 | −10 | gb105pln | Hordeum vulgare calreticulin (CRH1) mRNA, partial cds. | |
| 700380090H1 | g1054677 | 28 | −3 | gb105eukp | coxIII; cytochrome oxidase | |
| 700346932H1 | g886739 | 51 | −46 | gb105pln | Z. mays histone H4 gene. | |
| 700343049H1 | g576507 | 21 | −17 | gb105eukp | IAP75; component of chloroplast outer membrane protein import machinery; outer | |
| | | | | | membrane protein | |
| 700343076H1 | g1008185 | 7 | 6 | gb105eukp | MTR4 | |
| 700345689H1 | g2780344 | 24 | −3 | gb105pln | Arabidopsis thaliana gene for inorganic phosphate transporter, complete cds. | |
| 700345058H1 | g2073374 | 60 | −56 | gb105pln | Rice mRNA for farnesyl pyrophosphate synthase, complete cds. | |
| 700345696H1 | g717189 | 64 | 2 | gb105allp | RNA polymerase II subunit | |
| 700344182H1 | g1928990 | 41 | −30 | gb105pln | Citrullus lanatus heat shock protein 70 precursor mRNA, complete cds. | |
| 700343113H1 | g2760169 | 28 | −7 | gb105pln | Arabidopsis thaliana genomic DNA, chromosome 5, P1 clone: MFB13, complete | |
| | | | | | sequence. | |
| 700343807H1 | g1841398 | 35 | −25 | gb105pln | A. thaliana mRNA for AtTFIIB2 protein. | |
| 700344369H1 | g971699 | 8 | −2 | gb105allp | ribosomal protein S7 | |
| 700343892H1 | g170746 | 54 | −44 | gb105pln | Wheat histone H4 TH091 gene, complete cds. | |
| 700281960H2 | g2244788 | 21 | −3 | gb105pln | Arabidopsis thaliana DNA chromosome 4, ESSA I contig fragment No. 1. | |
| 700346736H1 | g391605 | 21 | 4 | gb105allp | casein kinase II catalytic subunit | |
| 700343819H1 | g21794 | 57 | −48 | gb105pln | Wheat histone H4 gene. | |
| 700346876H1 | g2224750 | 19 | −13 | gb105pln | Arabidopsis thaliana mRNA for ribosomal protein S6. | |
| 700346020H1 | g5037 | 26 | −4 | gb105pln | Yeast (Schizosaccharomyces pombe) mRNA for a ras-related GTP-binding protein. | |
| 700379729H1 | g416148 | 53 | −40 | gb105pln | Zea mays beta-7 tubulin (tub7) mRNA, complete cds. | |
| 700343432H1 | g2369765 | 42 | −32 | gb105pln | Citrus paradisi mRNA for hypothetical protein. | |
| 700347240H1 | g2459406 | 42 | −29 | gb105pln | Arabidopsis thaliana chromosome II BAC F4P9 genomic sequence, complete sequence. | |
| 700345993H1 | g474002 | 52 | −29 | gb105pln | Rice mRNA, partial homologous to ribosomal protein L39 gene. | |
| 700345077H1 | g2618605 | 34 | −23 | gb105pln | Arabidopsis thaliana genomic DNA, chromosome 5, P1 clone: MUK11, complete | |
| | | | | | sequence. | |
| 700343005H1 | g600768 | 52 | −37 | gb105pln | Oryza sativa cyclophilin 2 (Cyp2) mRNA, complete cds. | |
| 700345557H1 | g2668743 | 39 | −34 | gb105pln | Zea mays ubiquitin conjugating enzyme (UBC) mRNA, complete cds. | |
| 700345123H1 | g20163 | 51 | −46 | gb105pln | O. sativa Rr15 mRNA for 5S ribosomal RNA. | |
| 700345378H1 | g2460124 | 16 | 5 | gb105allp | putative transcription factor CA150 | |
| 700343709H1 | g459197 | 23 | −4 | gb105pln | Gossypium hirsutum vacuolar H+-ATPase subunit B mRNA, complete cds. | |
| 700342754H1 | g1519250 | 55 | −41 | gb105pln | Oryza sativa GF14-c protein mRNA, complete cds. | |
| 700343829H1 | g397395 | 64 | −43 | gb105pln | Z. mays MNB1b mRNA for DNA-binding protein. | |
| 700344840H1 | g22483 | 39 | −43 | gb105pln | Z. mays RNA for superoxide dismutase Sod4. | |
| 700347238H1 | g18330 | 18 | −0 | gb105eukp | CARSR8; glutathione s-transferase | |
| 700344042H1 | g21794 | 69 | −58 | gb105pln | Wheat histone H4 gene. | |
| 700281942H2 | g394754 | 16 | 4 | gb105pln | S. oleracea atpG mRNA for chloroplast CF(o)II ATP synthase subunit 9. | |
| 700379679H1 | g432367 | 60 | −54 | gb105pln | Rice mRNA for elongation factor 1 beta, complete cds. | |
| 700345729H1 | g984964 | 14 | −0 | gb105eukp | SIK1; suppressor of toxicity of GAL4-IKB; Sik1p | |
| 700344212H1 | g971283 | 33 | −14 | gb105pln | Rice mRNA for ribosomal protein S31, complete cds. | |
| 700347018H1 | g21228 | 40 | −15 | gb105eukp | 37 kD inner envelope membrane polypeptide | |
| 700345865H1 | g14199 | 30 | −11 | gb105pln | Petunia sp. mitochondrial rps19 gene for ribosomal protein S19. | |
| 700345971H1 | g1184775 | 60 | −49 | gb105pln | Zea mays glyceraldehyde-3-phosphate dehydrogenase GAPC4 (gpc4) mRNA, | |
| | | | | | complete cds. | |
| 700345575H1 | g602757 | 48 | −38 | gb105pln | Lycopersicon esculentum clp-like energy-dependent protease mRNA, complete cds. | |
| 700343596H1 | g22314 | 98 | −83 | gb105pln | Maize mRNA for GSH gluthatione S-transferase I (GST; EC 2.5.1.18). | |
| 700344163H1 | g2668743 | 70 | −52 | gb105pln | Zea mays ubiquitin conjugating enzyme (UBC) mRNA, complete cds. | |
| 700346344H1 | g2058313 | 18 | −6 | gb105eukp | CCR1; cinnamoyl-CoA reductase; EC 1.2.1.44 | |
| 700380711H1 | g1524120 | 40 | −38 | gb105pln | M. crystallinum mRNA for malate dehydrogenase. | |
| 700380076H1 | g928932 | 46 | −1 | gb105eukp | D18; sugar metabolism; homolog of dTDP-glucose 4-6-dehydratases | |
| 700345975H1 | g436030 | 61 | −4 | gb105eukp | 60S ribosomal protein L34 | |
| 700342384H1 | g972243 | 32 | −0 | gb105allp | ribosomal protein S5 | |
| 700281956H2 | g2274990 | 30 | −22 | gb105pln | Hordeum vulgare mRNA for expressed sequence tag. | |
| 700342732H1 | g1132482 | 72 | −70 | gb105pln | Rice mRNA for ADP-ribosylation factor, complete cds. | |
| 700345908H1 | g2827524 | 31 | −6 | gb105eukp | F8F16.110; predicted protein | |
| 700344132H1 | g1788486 | 10 | 3 | gb105allp | hypothetical 33.7 kD protein in nfo-fruA intergenic region | |
| 700344003H1 | g1360146 | 19 | −3 | gb105pln | Yeast (Saccharomyces cerevisiae) NBP35 gene. | |
| 700347063H1 | g529357 | 53 | −31 | gb105eukp | T12A2.2 | |
| 700346001H1 | g468732 | 33 | −29 | gb105pln | R. sativus (Fakir) APX mRNA for L-ascorbate peroxidase. | |
| 700344903H1 | g666094 | 12 | −2 | gb105eukp | DNA primase large chain (P58) | |
| 700343245H1 | g17935 | 40 | −25 | gb105pln | B. vulgaris mRNA for salt-inducible betaine aldehyde dehydrogenase. | |
| 700343926H1 | g1305457 | 16 | 4 | gb105eukp | ROC2; cytosolic cyclophilin | |
| 700346501H1 | g473602 | 35 | −23 | gb105pln | Zea mays W-22 histone H2A mRNA, complete cds. | |
| 700346621H1 | g455864 | 27 | −28 | gb105pln | CAS1 = cycloartenol synthase [Arabidopsis thaliana, mRNA, 2560 nt]. | |
| 700379919H1 | g168460 | 77 | −72 | gb105pln | Zea mays cyclophilin (CyP) mRNA, complete cds. | |
| 700346661H1 | g485376 | 63 | −60 | gb105pln | Zea mays alpha-3-tubulin gene, complete cds. | |
| 700345549H1 | g602605 | 34 | −12 | gb105pln | Zea mays tandem genes for alpha1-tubulin and alpha2-tubulin. | |
| 700343716H1 | g459894 | 69 | −70 | gb105pln | Zea mays sus1 gene, complete cds. | |
| 700342264H1 | g2641618 | 44 | −27 | gb105pln | Zea mays ubiquitin-conjugating enzyme protein E2 (ubc7) mRNA, complete cds. | |
| 700346319H1 | g454847 | 30 | −39 | gb105pln | Glycine max ribosomal protein S11 gene, complete cds. | |
| 700345013H1 | g1143863 | 23 | 6 | gb105pln | Oryza sativa beta-glucosidase mRNA, nucleaqr gene encoding chloroplast protein, | |
| | | | | | complete cds. | |
| 700380508H1 | g2088638 | 24 | −8 | gb105pln | Arabidopsis thaliana chromosome II BAC T28M21 genomic sequence, complete | |
| | | | | | sequence. | |
| 700346495H1 | g416265 | 17 | −1 | gb105pln | Rice mRNA for ribosomal protein A2, partial sequence. | |
| 700345727H1 | g2258468 | 45 | −56 | gb105pln | Oryza sativa replication protein A1 (Os-RPA1) mRNA, complete cds. | |
| 700343930H1 | g2599103 | 21 | −5 | gb105pln | Dualiella salina 60S ribosomal protein (DSRP1) mRNA, complete cds. | |
| 700346011H1 | g1004231 | 19 | 2 | gb105allp | seryl-tRNA synthetase | |
| 700343131H1 | g726471 | 34 | 0 | gb105eukp | unknown | |
| 700347156H1 | g287628 | 49 | −8 | gb105eukp | YS29A; ribosomal protein YS29 | |
| 700342284H1 | g156372 | 19 | −5 | gb105eukp | mlc-1; myosin light chain 1 | |
| 700347008H1 | g2760173 | 18 | −9 | gb105pln | Arabidopsis thaliana genomic DNA, chromosome 5, P1 clone: MYH19, complete | |
| | | | | | sequence. | |
| 700343144H1 | g1575127 | 87 | −54 | gb105pln | Zea mays lumenal binding protein cBiPe2 mRNA, complete cds. | |
| 700379726H1 | g496267 | 22 | −3 | gb105pln | Nicotiana tabacum GTP-binding protein (Ran-A1) mRNA, complete cds. | |
| 700345422H1 | g2145358 | 21 | −10 | gb105eukp | ATHB-9; transcription factor; HD-Zip protein | |
| 700344088H1 | g2264317 | 21 | −5 | gb105pln | Arabidopsis thaliana genomic DNA, chromosome 5, P1 clone: MUG13, complete | |
| | | | | | sequence. | |
| 700344032H1 | g2244760 | 23 | 8 | gb105allp | selenium-binding protein | |
| 700343859H1 | g2213621 | 37 | −14 | gb105eukp | F21J9.26 | |
| 700345990H1 | g2829887 | 18 | 5 | gb105allp | Hypothetical protein | |
| 700344203H1 | g516043 | 19 | −1 | gb105eukp | DWF1; Dwarf1 | |
| 700281860H2 | g168494 | 39 | 12 | gb105pln | Maize (Zea mays) histone H3 gene (H3C2), complete cds. | |
| 700345483H1 | g2058313 | 15 | −1 | gb105eukp | CCR1; cinnamoyl-CoA reductase; EC 1.2.1.44 | |
| 700346164H1 | g515759 | 15 | −2 | gb105eukp | malate dehydrogenase (NADP+); EC 1.1.1.40 | |
| 700380141H1 | g971830 | 42 | 3 | gb105allp | tubulin alpha-3 chain | |
| 700345223H1 | g4392 | 16 | −2 | gb105eukp | RPL37A; ribosomal protein L37a | |
| 700343508H1 | g2245096 | 13 | −3 | gb105eukp | inositol 2-dehydrogenase homolog | |
| 700345958H1 | g22484 | 32 | 3 | gb105pln | Z. mays RNA for superoxide dismutase Sod4A. | |
| 700345759H1 | g2662340 | 83 | −76 | gb105pln | Oryza sativa mRNA for EF-1 alpha, complete cds. | |
| 700346723H1 | g43030 | 19 | −1 | gb105allp | TAGI (aa 1-187) | |
| 700343702H1 | g1088346 | 8 | 5 | gb105allp | adenylate kinase | |
| 700342946H1 | g303852 | 58 | −47 | gb105pln | Rice mRNA for ribosomal protein L3, complete cds. | |
| 700343960H1 | g30293 | 18 | 2 | gb105allp | CTP synthetase (AA 1-591) | |
| 700343470H1 | g21796 | 65 | −42 | gb105pln | Wheat histone H3 gene. | |
| 700380518H1 | g20255 | 29 | −22 | gb105pln | O. sativa gene for heat shock protein 82 HSP82. | |
| 700346587H1 | g1903032 | 33 | −16 | gb105pln | B. napus mRNA for acyl-CoA synthetase (2360 bp). | |
| 700380781H1 | g21800 | 23 | −45 | gb105pln | T. aestivum L mRNA for histone H2B. | |
| 700346419H1 | g1930071 | 24 | −3 | gb105pln | Oryza sativa thioredoxin h mRNA, complete cds. | |
| 700344709H1 | g620041 | 31 | −34 | gb105pln | A. adeninivorans TEF gene for translation elongation factor EF-1alpha. | |
| 700342823H1 | g1136121 | 63 | −64 | gb105pln | O. sativa mRNA for alpha-tubulin (clone OSTA-136). | |
| 700343305H1 | g1742934 | 16 | 9 | gb105pln | A. bisporus rs13 mRNA for 40S ribosomal protein S13. | |
| 700347284H1 | g217827 | 29 | −5 | gb105eukp | AHBP-1b; AHBP-1b | |
| 700342726H1 | g2252864 | 9 | 2 | gb105allp | Similar to receptor kinase | |
| 700347150H1 | g2828296 | 32 | −10 | gb105eukp | T18B16.180; RNase L inhibitor | |
| 700343925H1 | g203066 | 18 | 4 | gb105allp | aspartyl-tRNA synthetase | |
| 700281852H2 | g2213884 | 25 | 5 | gb105eukp | lp-ipmsb; first enzyme in leucine biosynthesis which catalyzes condensation of acetyl- | |
| | | | | | CoA and 2-oxoisovalerate to form 2-isopropylmalate synthase; 2-isopropylmalate | |
| | | | | | synthase; EC 4.1.3.12 | |
| 700380284H1 | g398605 | 28 | −11 | gb105pln | A. thaliana gene for elongation factor 1 beta. | |
| 700345221H1 | g434905 | 22 | −15 | gb105pln | A. thaliana (Columbia) gene for S18 ribosomal protein (1471 bp). | |
| 700344044H1 | g406733 | 24 | 3 | gb105eukp | RpL18a; ribosomal protein L18a | |
| 700344035H1 | g2641200 | 54 | −26 | gb105pln | Fritillaria agrestis ribosomal protein L23a (rp123a) mRNA, complete cds. | |
| 700343663H1 | g1906812 | 56 | −24 | gb105allp | inducible carbonyl reductase | |
| 700380747H1 | g2293565 | 26 | −5 | gb105pln | Oryza sativa ADP-ribosylation factor 1 (Os-ARF1) mRNA, complete cds. | |
| 700344506H1 | g1255025 | 23 | 5 | gb105allp | FBP 11 | |
| 700342949H1 | g474002 | 46 | −23 | gb105pln | Rice mRNA, partial homologous to ribosomal protein L39 gene. | |
| 700346009H1 | g166857 | 57 | −46 | gb105pln | Arabidopsis thaliana cytoplasmic ribosomal protein mRNA, complete cds. | |
| 700380658H1 | g1143445 | 33 | 7 | gb105allp | cinnamyl alcohol dehydrogenase | |
| 700380490H1 | g168650 | 26 | −4 | gb105pln | Zea mays ubiquitin fusion protein (UBF9) gene, complete cds. | |
| 700346681H1 | g575730 | 24 | 1 | gb105pln | Z. mays mRNA for transmembrane protein. | |
| 700346162H1 | g601789 | 11 | −4 | gb105eukp | AFC2; protein kinase | |
| 700344852H1 | g531055 | 30 | −27 | gb105pln | Wheat mRNA for protein H2B-6, complete cds. | |
| 700379690H1 | g2218151 | 34 | −11 | gb105pln | Vigna unguiculata type IIa membrane protein cp-wap13 mRNA, complete cds. | |
| 700346005H1 | g902558 | 28 | 3 | gb105allp | acidic ribosomal phosphoprotein P1 | |
| 700344150H1 | g1783168 | 48 | −0 | gb105allp | CIRP | |
| 700344144H1 | g398328 | 30 | −10 | gb105pln | H. annuus mRNA for protein phosphatase type 2A. | |
| 700346392H1 | g2183249 | 19 | 2 | gb105eukp | glutathione-S-transferase | |
| 700345257H1 | g2811284 | 13 | 6 | gb105allp | ribosomal protein S7 | |
| 700343012H1 | g22322 | 17 | −16 | gb105pln | Z. mays mRNA for H2B histone (clone cH2B214). | |
| 700380053H1 | g170784 | 20 | 15 | gb105pln | Wheat ubiquitin carrier protein (UBC1) mRNA, complete cds. | |
| 700345320H1 | g1718497 | 26 | −18 | gb105eukp | ARP3; actin-related protein 3 | |
| 700343907H1 | g2435529 | 12 | −3 | gb105eukp | C10E2.3 | |
| 700342567H1 | g2281103 | 14 | 4 | gb105eukp | F18O19.22; Glucan endo-1,3-beta glucosidase isolog | |
| 700344634H1 | g485273 | 28 | −10 | gb105eukp | CDC46; Cdc46p | |
| 700345971H1 | g312180 | 31 | −50 | gb105pln | Z. mays GapC4 gene. | |
| 700346064H1 | g2351382 | 10 | 5 | gb105allp | eIF3-p48 | |
| 700345318H1 | g2832699 | 8 | 8 | gb105eukp | receptor serine/threonine kinase-lie protein | |
| 700342578H1 | g170228 | 32 | −31 | gb105pln | N. tabacum 5-enolpyruvylshikimate-3-phosphate synthase mRNA, complete cds. | |
| 700379915H1 | g442507 | 27 | −11 | gb105pln | Yeast (Saccharomyces pombe) (972) mts2 mRNA for 26S protease subunit. | |
| 700347003H1 | g2274983 | 29 | −27 | gb105pln | Hordeum vulgare mRNA for expressed sequence tag. | |
| 700344151H1 | g1946367 | 7 | 6 | gb105eukp | T06B20.13 | |
| 700343086H1 | g758352 | 74 | −74 | gb105pln | Z. mays mRNA for cysteine synthase. | |
| 700345506H1 | g1375074 | 25 | 0 | gb105pln | Oryza sativa mRNA for glyoxysomal malate dehydrogenase, complete cds. | |
| 700343276H1 | g395156 | 28 | −14 | gb105pln | Yeast (Saccharomyces cerevisiae) PRP8 gene, complete CDS. | |
| 700345535H1 | g1301694 | 12 | 4 | gb105eukp | F55A11.4 | |
| 700342262H1 | g968902 | 13 | −2 | gb105eukp | ribosomal protein S8 | |
| 700342582H1 | g2632109 | 19 | −0 | gb105eukp | phospholipid glutathione peroxidase | |
| 700380574H1 | g2570508 | 32 | −49 | gb105pln | Oryza sativa transmembrane protein mRNA, complete cds. | |
| 700347393H1 | g1619299 | 22 | −12 | gb105pln | L. esculentum LRP gene. | |
| 700342301H1 | g1008880 | 43 | −33 | gb105pln | Phaseolus vulgaris eukaryotic initiation factor 5 (eIF-5) gene, complete cds. | |
| 700346364H1 | g169820 | 56 | −34 | gb105pln | Oryza sativa triosephosphate isomerase (Rictpi) mRNA, complete cds. | |
| 700346181H1 | g21794 | 49 | 14 | gb105pln | Wheat histone H4 gene. | |
| 700342247H1 | g22651 | 21 | −14 | gb105pln | H. vulgare mRNA for GRP94 homologue. | |
| 700346739H1 | g2407273 | 9 | −3 | gb105eukp | lipid transfer protein LPT II | |
| 700347050H1 | g2244788 | 17 | 14 | gb105pln | Arabidopsis thaliana DNA chromosome 4, ESSA I contig fragment No. 1. | |
| 700346159H1 | g1546918 | 22 | −26 | gb105pln | Z. mays mRNA for translation initiation factor 5A. | |
| 700343696H1 | g2388783 | 15 | 6 | gb105allp | DRING protein | |
| 700346276H1 | g2827714 | 12 | 4 | gb105eukp | F6H11.160; receptor protein kinase-like protein | |
| 700346119H1 | g217973 | 32 | −61 | gb105pln | Zea mays gene for triosephosphate isomerase, complete cds. | |
| 700346017H1 | g2582065 | 65 | 2 | gb105allp | NADH dehydrogenase subunit 2 | |
| 700344139H1 | g168419 | 92 | −79 | gb105pln | Maize (Z. mays) aldolase mRNA, complete cds. | |
| 700344143H1 | g535770 | 26 | −5 | gb105pln | Nicotiana tabacum (clone: SP-3) dehydroquinate dehydratase/shikimate dehydrogenase | |
| | | | | | (aroD-E) mRNA, 3′ end. | |
| 700346820H1 | g1724099 | 26 | −4 | gb105pln | Mesembryanthemum crystallinum voltage-dependent anion-selective channel protein | |
| | | | | | porin (VDAC) mRNA, complete cds. | |
| 700343163H1 | g1707009 | 8 | −1 | gb105eukp | T01B08.11; Squamosa-promoter binding protein 1 isolog | |
| 700347192H1 | g1497983 | 44 | −20 | gb105eukp | alien; putative thyroid receptor interacting protein | |
| 700346704H1 | g2062605 | 5 | 5 | gb105allp | brain secretory protein hSec10p | |
| 700343151H1 | g1323459 | 25 | −10 | gb105pln | Yeast (Saccharomyces cerevisiae) chromosome VII reading frame ORF YGR253c. | |
| 700346255H1 | g2702281 | 30 | −20 | gb105eukp | T21L14.14; putative protein disulfide isomerase precursor | |
| 700342652H1 | g2351373 | 60 | −33 | gb105pln | Arabidopsis thaliana putative 26S proteasome subunit athMOV34 mRNA, complete cds. | |
| 700345723H1 | g2274990 | 46 | −60 | gb105pln | Hordeum vulgare mRNA for expressed sequence tag. | |
| 700345693H1 | g312180 | 31 | −44 | gb105pln | Z. mays GapC4 gene. | |
| 700346211H1 | g1483173 | 8 | 5 | gb105eukp | high mobility group protein | |
| 700344516H1 | g2330821 | 17 | −3 | gb105eukp | SPAC4F8.04; hypothetical protein | |
| 700379995H1 | g1931630 | 55 | −31 | gb105pln | Zea mays glutamate dehydrogenase (GDH1) mRNA, complete cds. | |
| 700347252H1 | g20255 | 63 | −57 | gb105pln | O. sativa gene for heat shock protein 82 HSP82. | |
| 700344325H1 | g1653904 | 8 | 5 | gb105allp | tRNA pseudouridine 55 synthase | |
| 700281862H2 | g633940 | 7 | 4 | gb105eukp | PTR2-B; peptide transporter; transport protein | |
| 700346779H1 | g1498257 | 20 | −10 | gb105allp | replication factor C, 36-kKa subunit | |
| 700343946H1 | g2760165 | 26 | −11 | gb105pln | Arabidopsis thaliana genomic DNA, chromosome 5, P1 clone: MAC9, complete | |
| | | | | | sequence. | |
| 700343762H1 | g19874 | 17 | −7 | gb105pln | N. tabacum mRNA for glutamate-1-semialdehyde aminotransferase. | |
| 700343663H1 | g666087 | 56 | −25 | gb105allp | carbonyl reductase (NADPH) | |
| 700343238H1 | g168543 | 72 | −23 | gb105pln | Zea mays putative ribosomal protein S8 mRNA, partial cds. | |
| 700380765H1 | g2511594 | 39 | −9 | gb105eukp | prcd; multicatalytic endopeptidase complex, proteasome precursor, beta subunit, | |
| | | | | | EC 3.4.99.46 | |
| 700347111H1 | g699510 | 25 | 1 | gb105allp | minute(1)1B protein | |
| 700344112H1 | g168494 | 55 | −29 | gb105pln | Maize (Zea mays) histone H3 gene (H3C2), complete cds. | |
| 700342243H1 | g2511590 | 23 | 1 | gb105allp | multicatalytic endopeptidase complex, proteasome component, beta subunit | |
| 700343858H1 | g540581 | 10 | 4 | gb105eukp | mudrA | |
| 700380608H1 | g664902 | 26 | −19 | gb105pln | C. plantagineum tkt7 gene for transketolase. | |
| 700281812H2 | g416150 | 85 | −57 | gg105pln | Zea mays beta-8 tubulin (tub8) mRNA, complete cds. | |
| 700344162H1 | g2511534 | 23 | 3 | gb105pln | Eleusine indica alpha-tubulin 3 (TUA3) mRNA, complete cds. | |
| 700342827H1 | g899235 | 38 | −21 | gb105eukp | F22B5.1 | |
| 700344953H1 | g2511589 | 22 | −0 | gb105pln | Arabidopsis thaliana mRNA for proteasome subunit prgb. | |
| 700344942H1 | g1350507 | 15 | −5 | gb105pln | Picea glauca mitochondria-localized low molecular weight heat shock protein 23.5 | |
| | | | | | (EMB22, SMW HSP23.5) mRNA, complete cds. | |
| 700380220H1 | g313135 | 30 | −18 | gb105pln | Z. mays mRNA for porin. | |
| 700343958H1 | g549975 | 8 | −0 | gb105eukp | nucleosome assembly protein I-like protein; similar to mouse nap I, PIR Accession | |
| | | | | | Number JS0707 | |
| 700346691H1 | g2529657 | 29 | −28 | gb105pln | Arabidopsis thaliana chromosome II BAC T30B22 genomic sequence, complete | |
| | | | | | sequence. | |
| 700344621H1 | g1086831 | 17 | −11 | gb105eukp | F10E7.7 | |
| 700345628H1 | g20255 | 36 | −25 | gb105pln | O. sativa gene for heat shock protein 82 HSP82. | |
| 700345489H1 | g434905 | 23 | −23 | gb105pln | A. thaliana (Columbia) gene for S18 ribosomal protein (1471 bp). | |
| 700346815H1 | g2262141 | 18 | −5 | gb105eukp | T10P11.8; hypothetical protein | |
| 700346018H1 | g1517920 | 27 | −15 | gb105eukp | transcription factor: Kruppel | |
| 700346992H1 | g2443473 | 41 | −19 | gb105eukp | SF2; ASF/SF2 homlog | |
| 700343344H1 | g861009 | 65 | −50 | gb105pln | Hordeum vulgare mRNA for bas1 protein. | |
| 700344418H1 | g16956 | 18 | −0 | gb105allp | 40S ribosomal protein S12 | |
| 700344259H1 | g1403206 | 14 | −6 | gb105eukp | F38B7.7 | |
| 700345755H1 | g1498337 | 54 | −49 | gb105pln | Glycine max actin (Soy119) gene, partial cds. | |
| 700343606H1 | g473986 | 17 | −23 | gb105pln | Rice mRNA, partial homologous to histone H2B gene. | |
| 700347138H1 | g1136119 | 70 | −60 | gb105pln | O. sativa mRNA for alpha-tubulin (clone OSTA-111). | |
| 700342713H1 | g2281081 | 18 | −11 | gb105pln | Arabidopsis thaliana chromosome II BAC F18O19 genomic sequence, complete | |
| | | | | | sequence. | |
| 700344201H1 | g1550813 | 40 | −76 | gb105pln | Z. mays mRNA for acidic ribosomal protein P0. | |
| 700343696H1 | g2239144 | 16 | 5 | gb105allp | transcription repressor Ring1A | |
| 700345693H1 | g1184775 | 64 | −44 | gb105pln | Zea mays glyceraldehyde-3-phosphate dehydrogenase GAPC4 (gpc4) mRNA, | |
| | | | | | complete cds. | |
| 700343256H1 | g2664199 | 21 | −16 | gb105pln | Arabidopsis thaliana GTL2 gene. | |
| 700346708H1 | g21802 | 55 | −59 | gb105pln | T. asestivum mRNA for high mobility group protein (HMGW). | |
| 700281968H2 | g1051262 | 8 | −3 | gb105eukp | RPL39; ribosomal protein L39 | |
| 700380124H1 | g30381 | 12 | 5 | gb105allp | cytochrome oxidase subunit VIb | |
| 700346892H1 | g514945 | 90 | −66 | gb105pln | Zea mays sucrose synthase (Sus1) mRNA, complete cds. | |
| 700346715H1 | g1419369 | 67 | −83 | gb105pln | Z. mays ZmABP3 mRNA for actin depolymerizing factor. | |
| 700343657H1 | g2407800 | 17 | 3 | gb105eukp | H2Av; histone H2A.F/Z | |
| 700342481H1 | g290275 | 18 | −9 | gb105eukp | M(3)95A; multifunctional activity and location; ribosomal protein S3/AP endonuclease | |
| | | | | | DNA repair protein | |
| 700379959H1 | g1061069 | 10 | 6 | gb105eukp | F54F7.1 | |
| 700347282H1 | g1906827 | 16 | −15 | gb105pln | A. thaliana hsp81.4 gene. | |
| 700346238H1 | g561663 | 42 | −47 | gb105pln | Rice mRNA, partial homologous to ribosomal protein coding sequence. | |
| 700345815H1 | g2645069 | 6 | 6 | gb105eukp | W09D10.2 | |
| 700346547H1 | g474009 | 60 | −56 | gb105pln | Rice mRNA, partial homologous to ribosomal protein S19 gene. | |
| 700343932H1 | g168460 | 71 | −80 | gb105pln | Zea mays cyclophilin (CyP) mRNA, complete cds. | |
| 700342967H1 | g21796 | 66 | −54 | gb105pln | Wheat histone H3 gene. | |
| 700343116H1 | g2599092 | 12 | 7 | gb105allp | WD-40 repeat protein MSI4 | |
| 700346535H1 | g998429 | 39 | −20 | gb105pln | GRF1 = general regulatory factor [Zea mays, XL80, Genomic, 5348 nt]. | |
| 700342288H1 | g2286110 | 17 | −6 | gb105pln | Oryza sativa MADS box protein (OsMADS7) mRNA, complete cds. | |
| 700344768H1 | g336633 | 26 | 4 | gb105eukp | elongation factor Tu (tufA) | |
| 700345658H1 | g1652063 | 16 | 2 | gb105allp | hypothetical protein | |
| 700380479H1 | g49652 | 14 | 8 | gb105allp | ribosomal protein S19 (AA 1-133) | |
| 700344836H1 | g170784 | 43 | −22 | gb105pln | Wheat ubiquitin carrier protein (UBC1) mRNA, complete cds. | |
| 700343713H1 | g732949 | 18 | −4 | gb104eukp | SSM1a | |
| 700342606H1 | g1665767 | 24 | 0 | gb105allp | Similar to Human KIAA0188 protein | |
| 700347133H1 | g2801433 | 51 | 0 | gb105eukp | Salt stress inducible small GTP binding protein Ran1 homolog | |
| 700344116H1 | g22328 | 58 | −46 | gb105pln | Maize mRNA for a high mobility group protein. | |
| 700342945H1 | g516043 | 27 | −4 | gb105eukp | DWF1; Dwarf1 | |
| 700342782H1 | g1617268 | 20 | −8 | gb105eukp | Acyl CoA synthetase; EC 6.2.1.3 | |
| 700343146H1 | g1848290 | 36 | −11 | gb105pln | Arabidopsis thaliana LON protease homolog (LON_ARA) mRNA, nuclear gene | |
| | | | | | encoding mitochonodrial protein, complete cds. | |
| 700343652H1 | g509810 | 28 | −9 | gb105eukp | PEA1; envelope Ca2+-ATPase | |
| 700343649H1 | g22314 | 66 | −78 | gb105pln | Maize mRNA for GSH gluthatione S-transferase I (GST; EC 2.5.1.18). | |
| 700346720H1 | g452711 | 27 | −14 | gb105pln | A. officinalis mRNA for beta-galactosidase. | |
| 700346241H1 | g2341031 | 36 | −21 | gb105eukp | F19P19.10; F19P19.10 | |
| 700344906H1 | g289658 | 53 | −29 | gb105eukp | C50C3.6 protein | |
| 700346126H1 | g757534 | 32 | −10 | gb105eukp | ara12; subtilisin-like protease | |
| 700345912H1 | g2330764 | 20 | −2 | gb105pln | Yeast (Saccharomyces pombe) chromosome I cosmid c23C11. | |
| 700342343H1 | g1913999 | 24 | −1 | gb105eukp | C10C6.6 | |
| 700347382H1 | g2282583 | 98 | −84 | gb105pln | Zea mays elongation factor 1-alpha (EF1-A) mRNA, complete cds. | |
| 700345913H1 | g1244773 | 14 | −1 | gb105eukp | RPL37A; Rp137ap: 60S ribosomal protein L37a | |
| 700344450H1 | g1546918 | 54 | 3 | gb105pln | Z. mays mRNA for translation initiation factor 5A. | |
| 700344327H1 | g1008880 | 9 | 16 | gb105pln | Phaseolus vulgaris eukaryotic initiation factor 5 (eIF-5) gene, complete cds. | |
| 700344217H1 | g436782 | 22 | −6 | gb105pln | Rice mRNA for cyc07, complete cds. | |
| 700343627H1 | g166929 | 22 | 11 | gb105pln | A. thaliana ubiquitin extension protein (UBQ1) gene, complete cds. | |
| 700281809H2 | g2341060 | 50 | −53 | gb105pln | Zea mays translational initiation factor eIF-4A (tif-4A3) mRNMA, | |
| | | | | | complete cds. | |
| 700346932H1 | g170746 | 51 | −47 | gb105pln | Wheat histone H4 TH091 gene, complete cds. | |
| 700344919H1 | g564023 | 27 | −39 | gb105pln | Zea mays dihydrolipoamide acetyl transferase (ZM/DHLAAT) mRNA, complete cds. | |
| 700343701H1 | g21834 | 41 | −40 | gb105pln | Wheat mRNA for cytosolic phosphoglycerate kinase (EC 2.7.2.3). | |
| 700379629H1 | g857573 | 13 | 17 | gb105pln | Oryza sativa vauolar H+-ATPase (vatp-P1) mRNA, complete cds. | |
| 700344980H1 | g459199 | 18 | 8 | gb105pln | Gossypium hirsutum vacuolar H+-ATPase subunit B mRNA, partial cds. | |
| 700343493H1 | g22144 | 95 | −8 | gb105pln | Maize anaerobically regulated gene for fructose bisphosphate aldolase (EC 4.1.2.13). | |
| 700346975H1 | g565643 | 7 | 7 | gb105allp | hnRNP B1 protein | |
| 700342537H1 | g170775 | 79 | −66 | gb105pln | Wheat translation elongation factor 1 alpha-subunit (TEF1) mRNA, complete cds. | |
| 700342672H1 | g1325967 | 23 | −10 | gb105pln | T. aestivum histone H2A gene (clone TH274). | |
| 700346549H1 | g487932 | 18 | 3 | gb105allp | Yhr088wp | |
| 700346077H1 | g710626 | 21 | −2 | gb105eukp | ERD15; ERD15 protein | |
| 700380248H1 | g1498052 | 90 | −81 | gb105pln | Zea mays ribosomal protein S8 mRNA, complete cds. | |
| 700346714H1 | g1408473 | 8 | 7 | gb105eukp | ADF2; actin depolymerizing factor 2 | |
| 700345650H1 | g2276349 | 24 | −9 | gb105pln | Yeast (Saccaromyces pombe) chromosome II cosmid c30D10. | |
| 700345652H1 | g203605 | 54 | 1 | gb105allp | contiguous repeat polypeptide | |
| 700345983H1 | g312178 | 35 | −68 | gb105pln | Z. mays GapC2 gene. | |
| 700343829H1 | g459267 | 39 | −25 | gb105pln | Z. mays gene for HMG protein. | |
| 700380093H1 | g1272684 | 26 | −21 | gb105pln | Z. mays mRNA for acetyl CoA carboxylase (partial). | |
| 700379721H1 | g1225912 | 8 | 2 | gb105eukp | crm1-N1; crm1-N1 protein | |
| 700346592H1 | g2252841 | 11 | 3 | gb105eukp | A_IG005I10.22 | |
| 700343550H1 | g173235 | 13 | 5 | gb105eukp | YL3; 5S ribosomal RNA binding protein | |
| 700345878H1 | g22237 | 96 | −79 | gb105pln | Maize mRNA for cytosolic GAPDH (GapC) glyceraldehyde-3-phosphate dehydrogenase. | |
| 700345723H1 | g968901 | 42 | −45 | gb105pln | Rice mRNA for ribosomal protein S8, complete cds. | |
| 700343034H1 | g973313 | 8 | 8 | gb105eukp | myo-inositol 1-phosphate synthase isozyme-2 | |
| 700346548H1 | g402961 | 11 | 5 | gb105allp | ribosomal protein 5 large subunit | |
| 700346303H1 | g218088 | 22 | −9 | gb105pln | Rice mRNA for ribosomal protein 117 (249 gene), partial sequence. | |
| 700346220H1 | g1184773 | 59 | −78 | gb105pln | Zea mays glyceraldehyde-3-phosphate dehydrogenase GAPC3 (gpc3) mRNA, | |
| | | | | | complete cds. | |
| 700345807H1 | g406209 | 23 | 6 | gb105allp | ‘ferritin L-chain’ | |
| 700345169H1 | g499011 | 62 | −52 | gb105pln | S. vulgare SoAc1 mRNA. | |
| 700342536H1 | g886739 | 48 | −43 | gb105pln | Z. mays histone H4 gene. | |
| 700345581H1 | g166548 | 27 | −17 | gb105pln | Avena sativa vacuolar H+-ATPase 16 kDa proteolipid subunit (vatp-P1) mRNA, | |
| | | | | | complete cds. | |
| 700281812H1 | g460988 | 62 | −40 | gb105pln | O. sativa (Arborio) Beta Tubulin mRNA, clone OSTB-34. | |
| 700346888H1 | g777757 | 61 | −31 | gb105pln | Saccharum hybrid (clone SCUBI561) polyubiquitin mRNA, complete cds. | |
| 700346712H1 | g304346 | 22 | −12 | gb105eukp | ZK686.4 protein | |
| 700342841H1 | g458927 | 10 | 15 | gb105pln | Yeast (Saccharomyces cerevisiae) chromosome VIII cosmid 9998. | |
| 700345629H1 | g2183248 | 39 | −24 | gb105pln | Triticum aestivum glutathione-S-transferase mRNA, complete cds. | |
| 700342303H1 | g998429 | 47 | −54 | gb105pln | GRF1 = general regulatory factor [Zea mays, XL 80, Genomic, 5348 nt]. | |
| 700379837H1 | g168639 | 9 | 16 | gb105pln | Zea mays autonomous transposable element En-1 mosaic protein gene, complete cds. | |
| 700343521H1 | g975887 | 32 | −30 | gb105pln | Mesembryanthemum crystallinum myo-inositol-1-phosphate synthase mRNA, | |
| | | | | | complete cds. | |
| 700379620H1 | g1841307 | 18 | 1 | gb105pln | Fission Yeast mRNA for ribosomal protein S16 homolog, partial cds. | |
| 700346812H1 | g1490384 | 44 | −15 | gb105allp | ribosomal protein L6 | |
| 700344053H1 | g287834 | 85 | −60 | gb105pln | Z. mays yptm2 cDNA. | |
| 700343933H1 | g22483 | 50 | −63 | gb105pln | Z. mays RNA for superoxide dismutase Sod4. | |
| 700379982H1 | g1262433 | 19 | 6 | gb105allp | 26S protease subunit | |
| 700379779H1 | g1524386 | 27 | −8 | gb105pln | Z. mays high copy dispersed repeat DNA, ZHCR4. | |
| 700346379H1 | g600115 | 75 | −71 | gb105pln | Z. mays apx gene encoding cytosolic ascorbate peroxidase. | |
| 700345914H1 | g168514 | 29 | −8 | gb105pln | Z. mays c1 locus myb homologue cDNA, exons 1-3. | |
| 700344424H1 | g2827887 | 22 | −10 | gb105pln | Arabidopsis thaliana ATP-dependent Clp protease proteolytic subunit (clpP) mRNA, | |
| | | | | | nuclear gene encoding chloroplast protein, complete cds. | |
| 700347111H1 | g458972 | 21 | 2 | gb105allp | similar to yeast ribosomal protein YL39 | |
| 700343972H1 | g2660677 | 24 | −1 | gb105eukp | T19K24.17 | |
| 700343743H1 | g2662342 | 35 | −38 | gb105pln | Oryza sativa mRNA for EF-1 alpha, complete cds. | |
| 700345677H1 | g1181672 | 70 | −54 | gb105pln | Sorghum bicolor heat shock protein 70 cognate (hsc70) mRNA, partial cds. | |
| 700344512H1 | g2262177 | 23 | −6 | gb105eukp | F5J6.22; hypothetical protein similar to T18A10.3 | |
| 700342466H1 | g1667359 | 14 | 4 | gb105eukp | des1; Des-1 protein | |
| 700347091H1 | g498738 | 48 | −32 | gb105pln | H. vulgare (pMaW20) pseudo mRNA for beta-ketoacyl-ACP synthase (partial). | |
| 700379658H1 | g557681 | 68 | −60 | g105pln | Z. mays mRNA for ribosomal protein S28. | |
| 700342885H1 | g2351063 | 19 | 4 | gb105pln | Arabidopsis thaliana genomic DNA, chromosome 5, P1 clone: MCL19. | |
| 700342353H1 | g1710363 | 10 | −0 | gb105eukp | splicing factor; U2AF65 | |
| 700346220H1 | g312180 | 34 | −79 | gb105pln | Z. mays GapC4 gene. | |
| 700346210H1 | g1749838 | 12 | 8 | gb105allp | porline iminopeptidase | |
| 700345843H1 | g1906603 | 13 | 11 | gb105pln | Zea mays ACCase gene, intron containing colonist1 and colonist2 retrotransposons | |
| | | | | | and reverse transcriptase pseudogene, complete sequence. | |
| 700346105H1 | g289203 | 14 | −1 | gb105eukp | lipoxygenase; EC 1.13.11.12 | |
| 700379736H1 | g21794 | 17 | −10 | gb105pln | Wheat histone H4. gene. | |
| 700343413H1 | g2464848 | 13 | 16 | gb105pln | Arabidopsis thaliana DNA chromosome 4, ESSA I AP2 contig fragment No. 1. | |
| 700342504H1 | g2832679 | 21 | −1 | gb105eukp | T10I14.120; putative protein | |
| 700346634H1 | g2459406 | 37 | −26 | gb105pln | Arabidospis thaliana chromosome II BAC F4P9 genomic sequence, complete | |
| | | | | | sequence. | |
| 700342528H1 | g218180 | 21 | −16 | gb105pln | Rice mRNA for oryzain alpha (EC 3.4.22). | |
| 700379869H1 | g1498052 | 98 | −89 | gb105pln | Zea mays ribosomal protein S8 mRNA, complete cds. | |
| 700347352H1 | g2656024 | 14 | 12 | gb105pln | Arabidopsis thaliana genomic DNA, chromosome 5, TAC clone: K15E6. | |
| 700347390H1 | g1916867 | 16 | −5 | gb105eukp | who; muscle development; WHO | |
| 700346122H1 | g1236950 | 15 | −2 | gb105pln | Glycine max nucleoside diphosphate kinase mRNA, complete cds. | |
| 700345992H1 | g20163 | 70 | −59 | gb105pln | O. sativa Rr15 mRNA for 5S ribosomal RNA. | |
| 700344966H1 | g391876 | 34 | −42 | gb105pln | Rice mRNA for adenylate kinase. | |
| 700344660H1 | g971485 | 46 | −8 | gb105eukp | putative beta-galactosidase/galactanase; EC 3.2.1.23 | |
| 700343356H1 | g1184773 | 98 | −88 | gb105pln | Zea mays glyceraldehyde-3-phosphate dehydrogenase GAPC3 (gpc3) mRNA, | |
| | | | | | complete cds. | |
| 700342334H1 | g22155 | 87 | −73 | gb105pln | Z. mays mRNA for alpha-tubulin 5. | |
| 700346053H1 | g217973 | 35 | −62 | gb105pln | Z. mays gene for triosephosphate isomerase, complete cds. | |
| 700342916H1 | g168462 | 54 | −76 | gb105pln | Z. mays defective suppressor-mutator from the brittle-1-mutable allele. | |
| 700380409H1 | g304108 | 31 | −19 | gb105pln | Arabidopsis thaliana poly(A)-binding protein mRNA, complete cds. | |
| 700342480H1 | g391874 | 28 | −8 | gb105pln | Rice mRNA for 40S subunit ribosomal protein, complete cds. | |
| 700346719H1 | g20237 | 34 | −34 | gb105pln | O. sativa (rice) constitutive GOS2 gene. | |
| 700345992H1 | g1206016 | 25 | −5 | gb105pln | Yeast (Schizosaccharomyces pombe) ribosomal protein L5 gene, complete cds. | |
| 700345596H1 | g303501 | 24 | 11 | gb105pln | A. thaliana KatA mRNA for kinesin-like motor protein heavy chain, complete cds. | |
| 700344880H1 | g915192 | 34 | −33 | gb105pln | Chlamydomonas reinhardtii DNA for actin, complete cds. | |
| 700345724H1 | g397481 | 61 | −64 | gb105pln | A. thaliana hsc70 mRNA for heat shock cognate protein. | |
| 700380796H1 | g602252 | 71 | −60 | gb105pln | Zea mays enolase (eno2) mRNA, complete cds. | |
| 700346511H1 | g1070358 | 9 | 5 | gb105allp | oxalate oxidase-like protein or germin-like protein | |
| 700346146H1 | g2394431 | 14 | 3 | gb105eukp | F23F1.5 | |
| 700343819H1 | g168500 | 62 | −52 | gb105pln | Maize (Zea mays) histone H4 gene (H4C14), complete cds. | |
| 700342378H1 | g168505 | 53 | −37 | gb105pln | Zea mays histone H3 gene, complete cds. | |
| 700380162H1 | g170775 | 44 | −34 | gb105pln | Wheat translation elongation factor 1 alpha-subunit (TEF1) mRNA, complete cds. | |
| 700345546H1 | g458972 | 17 | −4 | gb105eukp | F37C12.4 | |
| 700281819H2 | g8484 | 91 | −2 | gb105eukp | RpL7; ribosomal protein | |
| 700343266H1 | g1419087 | 17 | 13 | gb105pln | N. plumbaginifolia mRNA for calcreticulin. | |
| 700342429H1 | g2065530 | 20 | −7 | gb105pln | Lycopersicon esculentum endo-1,4-beta-glucanase (Cel3) mRNA, complete cds. | |
| 700346057H1 | g577824 | 42 | −44 | gb105pln | Z. mays gene for H2B histone (gH2B3). | |
| 700380261H1 | g2160144 | 22 | −11 | gb105eukp | F19K23.13 | |
| 700343188H1 | g453188 | 25 | −21 | gb105pln | Z. mays acp mRNA for acyl carrier protein. | |
| 700345443H1 | g1806140 | 34 | −7 | gb105eukp | cdc2MsC | |
| 700346763H1 | g168460 | 89 | −80 | gb105pln | Zea mays cyclophilin (CyP) mRNA, complete cds. | |
| 700346908H1 | g600768 | 56 | −42 | gb105pln | Oryza sativa cyclophilin 2 (Cyp2) mRNA, complete cds. | |
| 700346469H1 | g577824 | 26 | −27 | gb105pln | Z. mays gene for H2B histone (gH2B3). | |
| 700342649H1 | g2431768 | 46 | 2 | gb105pln | Zea mays acidic ribosomal protein P1a (rpp1a) mRNA, complete cds. | |
| 700342652H1 | g2599116 | 20 | 6 | gb105pln | Hypocrea jecorina proteasome regulatory subunit 12 (prs12) gene, complete cds. | |
| 700379817H1 | g22314 | 53 | −20 | gb105pln | Maize mRNA for GSH gluthathione S-transferase I (GST; EC 2.5.1.18). | |
| 700343961H1 | g1302478 | 12 | 5 | gb105allp | ORF YN6006w | |
| 700343008H1 | g2828278 | 18 | 7 | gb105pln | Arabidopsis thaliana DNA chromosome 4, BAC clone T18B16 (ESSAII project). | |
| 700346366H1 | g169818 | 29 | −58 | gb105pln | Rice 25S ribosomal RNA gene. | |
| 700345254H1 | g2330648 | 28 | −15 | gb105pln | Pisum sativum mRNA for topoisomerase I. | |
| 700344709H1 | g168379 | 29 | −34 | gb105pln | M. racemosus TEF-1 gene encoding elongation factor 1-alpha (EF-1-alpha), | |
| | | | | | complete cds. | |
| 700343413H1 | g2464883 | 21 | 7 | gb105eukp | hypothetical protein | |
| 700347058H1 | g21800 | 22 | −32 | gb105pln | T. aestivum L mRNA for histone H2B. | |
| 700346839H1 | g1667582 | 17 | 6 | gb105allp | splicing factor At-SRp40 | |
| 700281870H2 | g1877523 | 22 | −27 | gb105pln | Arabidopsis thaliana BAC T7I23, complete sequence. | |
| 700342653H1 | g532788 | 15 | 4 | gb105eukp | caz; RNA binding protein; sarcoma associated RNA binding fly homologue | |
| 700346889H1 | g1397320 | 29 | −1 | gb105eukp | R08C7.3 | |
| 700345539H1 | g22176 | 13 | 15 | gb105pln | Z. mays P gene. | |
| 700345223H1 | g1244773 | 16 | −2 | gb105eukp | RPL37A; Rp137ap: 60S ribosomal protein L37a | |
| 700380665H1 | g599722 | 13 | 9 | gb105pln | C. melo mRNA for aconitase (UNI-ZAPxR). | |
| 700379664H1 | g166868 | 42 | −34 | gb105pln | Arabidopsis thaliana ribosomal protein S11 (RPS11-beta) mRNA, complete cds. | |
| 700380244H1 | g493590 | 64 | −58 | gb105pln | Hordeum vulgare disulfide isomerase (PDI) mRNA, 3′ end. | |
| 700345596H1 | g1438841 | 28 | −3 | gb105pln | Arabidopsis thaliana katB mRNA for heavy chain polypeptide of kinesin-like protein, | |
| | | | | | complete cds. | |
| 700344604H1 | g575291 | 65 | −59 | gb105pln | H. vulgare mRNA for SNF1-related protein kinase. | |
| 700346430H1 | g22430 | 17 | 11 | gb105pln | Maize pseudo-Gpa2 pseudogene for glyceraldehyde-3-phosphate dehydrogenase | |
| | | | | | subunit A. | |
| 700281822H2 | g169023 | 17 | 5 | gb105eukp | hsp70; 70 kDa heat shock protein | |
| 700281833H2 | g2570508 | 22 | −10 | gb105pln | Oryza sativa transmembrane protein mRNA, complete cds. | |
| 700344170H1 | g18259 | 21 | −7 | gb105pln | C. sativus mRNA for cs DnaJ-1. | |
| 700344120H1 | g1488296 | 18 | 5 | gb105pln | Oryza sativa osRAD23 mRNA, complete cds. | |
| 700281967H1 | g459894 | 54 | −78 | gb105pln | Zea mays sus1 gene, complete cds. | |
| 700379937H1 | g899607 | 96 | −84 | gb105pln | Zea mays polyubiquitin (MubC5) mRNA, complete cds. | |
| 700346838H1 | g1946689 | 22 | −1 | gb105pln | Arabidopsis thaliana glutathione peroxidase GPx mRNA, complete cds. | |
| 700380747H1 | g487286 | 38 | 3 | gb105pln | Rice mRNA EN053, partial sequence. | |
| 700346346H1 | g2108344 | 16 | −3 | gb105pln | Brassica campestris small GTP-binding protein Bsar1a (bsar1a) mRNA, complete cds. | |
| 700345454H1 | g1813684 | 8 | 5 | gb105eukp | bm-rbp-1; putative RNA binding protein | |
| 700379826H1 | g1002532 | 28 | −25 | gb105pln | Arabidopsis thaliana actin-11 (ACT11) gene, complete cds. | |
| 700347236H1 | g1749747 | 25 | −9 | gb105pln | Fission Yeast mRNA, partial cds. | |
| 700344352H1 | g300082 | 45 | −42 | gb105pln | hsp82 = 82 kda heat shock protein [Zea mays, seeding, leaves, Genomic, 3468 nt]. | |
| 700342985H1 | g886402 | 44 | −27 | gb105pln | Oryza sativa MADS-box protein (MADS4) mRNA, complete cds. | |
| 700347055H1 | g1905933 | 39 | −21 | gb105pln | Sorghum bicolor putative MADS box protein (SbMADS2) mRNA, partial cds. | |
| 700344843H1 | g218177 | 23 | −5 | gb105pln | Rice mRNA for ribosomal protein L35 (NH77 gene), partial sequence. | |
| 700343796H1 | g1079566 | 14 | 3 | gb105allp | Hep27 protein | |
| 700281887H2 | g968901 | 43 | −49 | gb105pln | Rice mRNA for ribosomal protein S8, complete cds. | |
| 700343026H1 | g1161230 | 12 | 7 | gb105allp | protocadherin-3 | |
| 700343287H1 | g939778 | 51 | −31 | gb105pln | Zea mays MADS-box protein (ZAG3) mRNA, complete cds. | |
| 700346533H1 | g644492 | 36 | −41 | gb105pln | Corn elongation factor 1alpha gene, complete cds. | |
| 700345868H1 | g1495251 | 6 | 7 | gb105eukp | heat-shock protein | |
| 700346705H1 | g22324 | 37 | −25 | gb105pln | Z. mays mRNA for H2B histone (clone cH2B221). | |
| 700281971H1 | g436030 | 44 | 3 | gb105allp | 60s ribosomal protein L34 | |
| 700346822H1 | g2244854 | 8 | 4 | gb105eukp | hypothetical protein | |
| 700346872H1 | g2231173 | 15 | 3 | gb105allp | cdc21p | |
| 700343443H1 | g1165205 | 18 | −14 | gb105pln | Arabidopsis thaliana multiubiquitin chain binding protein (MBP1) mRNA, complete cds. | |
| 700344505H1 | g30295 | 11 | 2 | gb105allp | cytochrome c oxidase subunit VIb (AA1--86) | |
| 700344029H1 | g2522362 | 30 | 1 | gb105allp | MutS homolog 2 | |
| 700343802H1 | g2821959 | 28 | −16 | gb105eukp | spermidine synthase; EC 2.5.1.6 | |
| 700281929H2 | g1930080 | 27 | −15 | gb105pln | Garcinia mangostana acyl-ACP thioesterase (FatB1) mRNA, nuclear gne encoding | |
| | | | | | chloroplast protein, complete cds. | |
| 700342244H1 | g1292866 | 26 | 4 | gb105allp | por1 | |
| 700380788H1 | g777757 | 53 | −40 | gb105pln | Saccharum hybrid (clone SCUBI561) polyubiquitin mRNA, complete cds. | |
| 700344051H1 | g2196465 | 22 | 6 | gb105pln | Arabidopsis thaliana mRNA for TATA binding protein-associated factor. | |
| 700343409H1 | g1103727 | 36 | −26 | gb105pln | Yeast (Saccharomyces pombe) chromosome I cosmid c1F5. | |
| 700345441H1 | g457708 | 38 | −25 | gb105pln | S. oleracea mRNA for protein kinase. | |
| 700343021H1 | g2641235 | 16 | −2 | gb105eukp | ACT1; actin | |
| 700342285H1 | g578845 | 17 | −11 | gb105eukp | COI; cytochrome oxidase subunit 1 | |
| 700342755H1 | g2244788 | 17 | 15 | gb105pln | Arabidopsis thaliana DNA chromosome 4, ESSA I contig fragment No. 1. | |
| 700379989H1 | g727357 | 15 | 0 | gb105eukp | atj; molecular chaperone; DnaJ homolog | |
| 700379623H1 | g2104681 | 27 | −10 | gb105eukp | transcription factor | |
| 700342823H1 | g22149 | 80 | −79 | gb105pln | Z. mays mRNA for alpha-tubulin 3. | |
| 700345985H1 | g2564336 | 18 | 6 | gb105pln | Crassica campestris mRNA for Tat binding protein 1, complete cds. | |
| 700345467H1 | g1374771 | 29 | −15 | gb105pln | Arabidopsis thaliana mRNA for diacylglycerol kinase, complete cds. | |
| 700380593H1 | g393399 | 37 | −39 | gb105pln | Z. mays Irma and En1 element DNA. | |
| 700346851H1 | g218130 | 13 | −2 | gb105pln | Rice mRNA for Ribosomal protein S15. | |
| 700345540H1 | g2662342 | 62 | −47 | gb105pln | Oryza sativa mRNA for EF-1 alpha, complete cds. | |
| 700342308H1 | g1498389 | 57 | −50 | gb105pln | Zea mays actin (Maz81) gene, partial cds. | |
| 700346666H1 | g1136119 | 79 | −65 | gb105pln | O. sativa mRNA for alpha-tubulin (clone OSAT-111). | |
| 700346495H1 | g790507 | 40 | −32 | gb105pln | Z. mays mRNA for 60S acidic robosomal protein. | |
| 700345013H1 | g1143864 | 29 | 3 | gb105allp | beta glucosidase | |
| 700344058H1 | g455995 | 71 | −32 | gb105allp | A-gamma globin | |
| 700342715H1 | g1323402 | 14 | −0 | gb105eukp | ORF YGR223c | |
| 700344914H1 | g166857 | 55 | −48 | gb105pln | Arabidopsis thaliana cytoplasmic ribosomal protein mRNA, complete cds. | |
| 700380283H1 | g886737 | 59 | −47 | gb105pln | Z. mays histone H3 gene. | |
| 700346994H1 | g2245014 | 11 | 3 | gb105eukp | glucosyltransferase homolog | |
| 700344042H1 | g168500 | 72 | −59 | gb105pln | Maize (Zea mays) histone H4 gene (H4C14), complete cds. | |
| 700343961H1 | g496723 | 12 | 5 | gb105eukp | N2038 | |
| 700343014H1 | g2828280 | 26 | −11 | gb105eukp | T18B16.20; putative protein | |
| 700343566H1 | g22747 | 12 | 17 | gb105pln | L. longiflorum mRNA for actin depolymerizing factor. | |
| 700379753H1 | g2414636 | 11 | 7 | gb105eukp | SPAC3A11.08; hypothetical protein | |
| 700345468H1 | g1181458 | 6 | 6 | gb105allp | similar to Mycobacterium ORF, corresponds to GenBank Accession Number Z79701 | |
| 700344445H1 | g1438062 | 15 | −5 | gb105allp | PWP2 | |
| 700345741H1 | g188565 | 71 | −20 | gb105allp | mitochondrial hinge protein precursor | |
| 700347163H1 | g829221 | 15 | 8 | gb105eukp | histone H2 A.F/Z | |
| 700281942H2 | g394755 | 22 | −6 | gb105eukp | CF(o)II ATP synthase subunit 9 | |
| 700346518H1 | g21531 | 34 | −34 | gb105pln | S. tuberosum PoAc100 gene for actin (exons 2, 3, & 4). | |
| 700343654H1 | g42800 | 11 | 5 | gb105allp | ribosomal protein L17 (aa 1-127) | |
| 700344114H1 | g643596 | 83 | −55 | gb105pln | Corn mRNA for cysteine proteinase, clone CCP, complete cds. | |
| 700343945H1 | g1519250 | 54 | −37 | gb105pln | Oryza sativa GF14-c protein mRNA, complete cds. | |
| 700343625H1 | g451489 | 16 | −2 | gb105allp | phenylethylamine oxidase; monoamine oxidase | |
| 700345472H1 | g17391 | 14 | 2 | gb105allp | GLYCINE-RICH RNA-BINDING PROTEIN | |
| 700346921H1 | g886739 | 58 | −40 | gb105pln | Z. mays histone H4 gene. | |
| 700346686H1 | g2599103 | 21 | 1 | gb105pln | Dunaliella salina 60S ribosomal protein (DSRP1) mRNA, complete cds. | |
| 700345277H1 | g169127 | 36 | −34 | gb105pln | Pisum sativum (clone pCLp) nuclear encoded precursor to chloroplast protein mRNA, | |
| | | | | | complete cds. | |
| 700344993H1 | g168722 | 44 | −57 | gb105pln | Z. mays protein phosphatase-1 (ZmPP1) mRNA, complete cds. | |
| 700345771H1 | g402903 | 27 | −23 | gb105pln | Arabidopsis thaliana Columbia laminin receptor-like protein mRNA, complete cds. | |
| 700342307H1 | g1913999 | 35 | −17 | gb105eukp | C10C6.6 | |
| 700346740H1 | g1195531 | 21 | 2 | gb105allp | type I keratin 16, K16 [human, epidermal keratinocytes, Peptide, 473 aa] | |
| 700343112H1 | g2760165 | 33 | −13 | gb105pln | Arabidopsis thaliana genomic DNA, chromosome 5, P1 clone: MAC9, complete | |
| | | | | | sequence. | |
| 700346901H1 | g398844 | 70 | −69 | gb105pln | Z. mays mRNA for beta 3 tubulin. | |
| 700346092H1 | g555945 | 18 | 0 | gb105allp | ribosomal protein S3 | |
| 700345553H1 | g2160184 | 16 | −6 | gb105eukp | F21M12.6 | |
| 700345465H1 | g166745 | 35 | −25 | gb105pln | A. thaliana plasma membrane proton ATPase (PMA) mRNA, complete cds. | |
| 700344536H1 | g2160158 | 23 | 6 | gb105allp | Similar to elongation factor 1-gamma (gb\|EF1G_XENLA). ESTs gb\|T20564, gb\|T45940, | |
| | | | | | gb\|T04527 come from this gene. | |
| 700380480H1 | g416432 | 32 | 2 | gb105allp | ubiquitin-conjugating enzyme E2-17 kD | |
| 700343874H1 | g2654088 | 24 | −12 | gb105eukp | KUP1; potassium transporter | |
| 700379919H1 | g829147 | 81 | −76 | gb105pln | Z. mays gene for cyclophilin. | |
| 700343635H1 | g1419369 | 38 | −15 | gb105pln | Z. mays ZmABP3 mRNA for actin depolymerizing factor. | |
| 700342714H1 | g2384757 | 49 | −44 | gb105pln | Oryza sativa GDP dissociation inhibitor protein OsGDI1 (OsGDI1) mRNA, | |
| | | | | | complete cds. | |
| 700342678H1 | g2739382 | 12 | 4 | gb105eukp | T9J22.24; myosin heavy chain-like protein | |
| 700343084H1 | g520543 | 41 | −79 | gb105pln | Sorphum bicolor clone BADH1 betaine aldehyde dehydrogenase mRNA, partial cds. | |
| 700343108H1 | g474007 | 38 | −24 | gb105pln | Rice mRNA, partial homologus to ribosomal protein S12 gene. | |
| 700343204H1 | g415634 | 29 | −2 | gb105eukp | cloned by ability to arrest the cell cycle when expressed in the fission yeast | |
| | | | | | Schizosaccharomyces pombe; homolog of human 40S ribosomal protein S31 | |
| 700347019H1 | g1814402 | 51 | −43 | gb105pln | Mesembryanthemum crystallinum methionine synthase (MetE) mRNA, complete cds. | |
| 700346510H1 | g600710 | 14 | 2 | gb105eukp | SAR1 | |
| 700345369H1 | g2244991 | 25 | −5 | gb105pln | Arabidopsis thaliana DNA chromosome 4, ESSA I contig fragment No. 6. | |
| 700344420H1 | g1778820 | 13 | 2 | gb105pln | Oryza sativa S-adenosyl-L-methionine synthetase)pOS-SAMS2) mRNA, complete cds. | |
| 700347071H1 | g168460 | 67 | −56 | gb105pln | Zea mays cyclophilin (CyP) mRNA, complete cds. | |
| 700344840H1 | g22484 | 46 | −44 | gb105pln | Z. mays RNA for superoxide dismutase Sod4A. | |
| 700342567H1 | g2281104 | 7 | 8 | gb105eukp | F18O19.23; Glucan endo-1,3,-beta glucosidase isolog | |
| 700347362H1 | g2760334 | 23 | −5 | gb105eukp | F1N21.5 | |
| 700346319H1 | g170053 | 45 | −43 | gb105pln | Soybean ribosomal protein S11 mRNA, 3′ end. | |
| 700345321H1 | g218241 | 23 | −35 | gb105pln | Rice mRNA for ribosomal protein L3 (T82 gene), partial sequence. | |
| 700344815H1 | g1237249 | 24 | −8 | gb105pln | N. tabacum cytochrome P-450 gene. | |
| 700344767H1 | g2326946 | 43 | −60 | gb105pln | Z. mays mRNA for chlorophyll a/b-binding protein CP29. | |
| 700343927H1 | g1814236 | 32 | −14 | gb105eukp | ubaA; ubiquitin-activating enzyme | |
| 700281977H2 | g167107 | 45 | −42 | gb105pln | Hordeum vulgare vacuolar ATPase B subunit isoform mRNA, complete cds. | |
| 700346953H1 | g2264307 | 15 | −2 | gb105pln | Arabidopsis thaliana genomic DNA, chromosome 5, P1 clone: MED 24, complete | |
| | | | | | sequence. | |
| 700346325H1 | g310934 | 38 | −25 | gb105pln | Nicotiana tabacum ribosomal protein L25, complete cds. | |
| 700343842H1 | g2366750 | 14 | 7 | gb105eukp | RGP-3; RNA binding protein | |
| 700342885H1 | g407488 | 12 | 4 | gfb105eukp | unknown | |
| 700380182H1 | g732950 | 16 | −5 | gb105pln | Yeast (Saccharomyces cerevisiae) SSM1b gene. | |
| 700346988H1 | g431212 | 15 | 0 | gb105eukp | GFA1; glutamine-fructose-6-phosphate aminotransferase; YKL457 | |
| 700346488H1 | g2243115 | 34 | −21 | gb105pln | A. thaliana mRNA for aspartate kinase. | |
| 700346193H1 | g459894 | 44 | −6 | gb105pln | Zea mays sus1 gene, complete cds. | |
| 700345259H1 | g1620592 | 6 | 1 | gb105eukp | SmDLC; dynein light chain | |
| 700346231H1 | g1839334 | 12 | 7 | gb105allp | ribosomal protein L37 {C2-C2 zinc-finger-like} [human, HeLa cells, Peptide, 97 aa] | |
| 700344486H1 | g1907211 | 9 | 5 | gb105eukp | YCR072c, len:515 | |
| 700345058H1 | g662367 | 54 | −49 | gb105pln | Zea mays farnesyl pyrophosphate synthetase (fps) mRNA, complete cds. | |
| 700343334H1 | g17917 | 13 | −4 | gb105eukp | S-receptor kinase related protein | |
| 700344852H1 | g473604 | 23 | −18 | gb105pln | Zea mays W-22 histone H2B mRNA, complete cds. | |
| 700344292H1 | g398921 | 33 | −14 | gb105pln | B. napus cold induced protein (BnC24B). | |
| 700281929H2 | g804947 | 22 | −7 | gb105pln | A. thaliana mRNA for acyl-(acyl carrier protein) thioesterase. | |
| 700342505H1 | g17862 | 33 | −18 | gb105pln | B. napus mRNA for ribosomal protein S15a (pPcB8). | |
| 700343395H1 | g498738 | 44 | −26 | pb105pln | H. vulgare (pMaW20) pseudo mRNA for beta-ketoacyl-ACP synthase (partial). | |
| 700343920H1 | g1750404 | 31 | −8 | gb105eukp | B0412.4 | |
| 700379915H1 | g557598 | 24 | −11 | gb105pln | Yeast (Saccharomyces cerevisiae) ATPase gene, complete cds. | |
| 700379827H1 | g18259 | 48 | −43 | gb105pln | C. sativus mRNA for cs DnaJ-1. | |
| 700347236H1 | g2408013 | 25 | −9 | gb105pln | Yeast (Saccharomyces pombe) chromosome I cosmid c17G6. | |
| 700344387H1 | g1419467 | 12 | −9 | gb105eukp | ZC434.5 | |
| 700281915H1 | g1463121 | 24 | −4 | gb105eukp | RPS3; ribosomal protein S3 | |
| 700346463H1 | g2624199 | 37 | −27 | gb105pln | M. acuminata mRNA; clone pBAN UU93. | |
| 700344544H1 | g22328 | 77 | −68 | gb105pln | Maize mRNA for a high mobility group protein. | |
| 700345692H1 | g498774 | 38 | −58 | gb105pln | Z. mays (cv DH5xDH7) hsp70-5 mRNA for heat shock protein 70. | |
| 700344370H1 | g2366749 | 17 | 7 | gb105pln | Nicotiana sylvestris mRNA for RNA binding protein, complete cds. | |
| 700343927H1 | g1055197 | 28 | −14 | gb105eukp | F11H8.1 | |
| 700347105H1 | g2511572 | 34 | −8 | gb105eukp | prcga; multicatalytic endopeptidase; EC 3.4.99.46 | |
| 700343588H1 | g2196671 | 35 | −72 | gb105pln | Z. mays mRNA for HMG protein | |
| 700342769H1 | g1799916 | 6 | 2 | gb105allp | similar to [SwissProt Accession Number P36979] | |
| 700380186H1 | g1419684 | 29 | −14 | gb105pln | M. sativa mRNA for TCTP-like protein. | |
| 700346817H1 | g2668745 | 29 | −11 | gb105pln | Zea mays inorganic pyrophosphatase (IPP) mRNA, complete cds. | |
| 700346681H1 | g1054795 | 23 | 2 | gb105pln | H. vulgare mRNA for transmembrane protein. | |
| 700345505H1 | g644492 | 71 | −54 | gb105pln | Corn elongation factor 1alpha gene, complete cds. | |
| 700344052H1 | g410509 | 22 | −9 | gb105pln | Yeast (Saccharomyces cerevisiae) CIM5 gene for putative ATPase. | |
| 700379649H1 | g416265 | 26 | −12 | gb105pln | Rice mRNA for ribosomal protein A2, partial sequence. | |
| 700343651H1 | g2274990 | 39 | −35 | gb105pln | Hordeum vulgare mRNA for expressed sequence tag. | |
| 700342482H1 | g2347192 | 10 | 6 | gb105allp | spliceosomal protein U2B isolog | |
| 700345844H1 | g218160 | 36 | −15 | gb105pln | Oryza sativa mRNA for elongation factor 1 beta′. | |
| 700345376H1 | g2330693 | 14 | −2 | gb105eukp | SPAC16E8.04c; chorismate mutase | |
| 700342524H1 | g20255 | 20 | −18 | gb105pln | O. sativa gene for heat shock protein 82 HSP82. | |
| 700346835H1 | g1100224 | 35 | −22 | gb105pln | Pinus sylvestris chloroplast NAD+-dependent glyceraldehyde-3-phosphate | |
| | | | | | dehydrogenase mRNA, complete cds. | |
| 700346748H1 | g551482 | 13 | 3 | gb105pln | Zea mays ABA- and ripening-inducible-like protein mRNA, complete cds. | |
| 700379731H1 | g20255 | 29 | −31 | gb105pln | O. sativa gene for heat shock protein 82 HSP82. | |
| 700347114H1 | g169295 | 48 | −10 | gb105pln | Pharbitis nil heat shock protein 83 (Hsp83) gene, complete cds. | |
| 700379763H1 | g1200153 | 36 | −16 | gb105pln | S. tuberosum mRNA for starch synthase. | |
| 700342277H1 | g21800 | 21 | −40 | gb105pln | T. aestivum L mRNA for histone H2B. | |
| 700345441H1 | g457688 | 41 | −28 | gb105pln | M. crystallinum mRNA for protein kinase. | |
| 700345254H1 | g16557 | 35 | −23 | gb105pln | A. thaliana TOP1 mRNA for topoisomerase I. | |
| 700343160H1 | g1143863 | 41 | −25 | gb105pln | Oryza sativa beta-glucosidase mRNA, nuclear gene encoding chloroplast protein, | |
| | | | | | complete cds. | |
| 700342213H1 | g22324 | 22 | −5 | gb105pln | Z. mays mRNA for H2B histone (clone cH2B221). | |
| 700347059H1 | g2464876 | 29 | −6 | gb105eukp | patatin homolog | |
| 700346461H1 | g459894 | 54 | −56 | gb105pln | Zea mays sus1 gene, complete cds. | |
| 700343461H1 | g409070 | 16 | 6 | gb105allp | HBp15/L22 | |
| 700347255H1 | g780326 | 28 | −28 | gb105pln | Eucalyptus globulus bicostata symbiont (F00060) mRNA, ESTun038. | |
| 700345844H1 | g218340 | 38 | −17 | gb105pln | Triticum aestivum mRNA for elongation factor 1 beta′. | |
| 700343201H1 | g166848 | 26 | −12 | gb105eukp | receptor-like protein kinase; EC 2.7.11. | |
| 700344763H1 | g1223910 | 23 | −1 | gb105eukp | chaperonin-60 alpha subunit | |
| 700343922H1 | g289212 | 39 | −30 | gb105pln | Atriplex nummularia homologous sequence (ANJ1) mRNA. | |
| 700342211H1 | g1915974 | 15 | −7 | gb105eukp | FK; fructokinase; EC 2.7.1.4 | |
| 700342976H1 | g1632830 | 18 | 9 | gb105pln | R. communis mRNA (unknown). | |
| 700347311H1 | g1244773 | 13 | 3 | gb105eukp | RPL37A; Rp137ap: 60S ribosomal protein L37a | |
| 700346566H1 | g20805 | 23 | −3 | gb105pln | P. sativum lpd mRNA for dihydrolipoamide dehydrogenase. | |
| 700345336H1 | g2281103 | 14 | −0 | gb105eukp | F18O19.22; Glucan endo-1,3-beta glucosidase isolog | |
| 700343743H1 | g473976 | 33 | −45 | gb105pln | Rice mRNA, partial homologous to elongation factor 1-alpha gene. | |
| 700281923H2 | g21800 | 31 | −35 | gb105pln | T. aestivum L mRNA for histone H2B. | |
| 700281817H2 | g2460018 | 8 | 1 | gb105allp | RecA | |
| 700344854H1 | g624220 | 51 | −40 | gb105pln | Oryza sativa signal recognition particle 19 kDa protein subunit SRP19 mRNA, | |
| | | | | | complete cds. | |
| 700343641H1 | g407028 | 20 | 7 | gb105pln | Zea mays gene for Ferredoxin-NADP reductase binding protein. | |
| 700345158H1 | g871812 | 13 | 7 | gb105eukp | ARA.KIN | |
| 700343081H1 | g2827531 | 10 | 7 | gb105eukp | F8F16.180; hypothetical protein | |
| 700380121H1 | g21800 | 27 | −26 | gb105pln | T. aestivum L mRNA for histone H2B. | |
| 700347153H1 | g1769965 | 36 | −25 | gb105pln | B. napus mRNA for enoyl reductase. | |
| 700344961H1 | g2245073 | 23 | 1 | gb105pln | Arabidopsis thaliana DNA chromosome 4, ESSA I contig fragment No. 8. | |
| 700347186H1 | g431167 | 42 | −26 | gb105pln | Lily mRNA for RAD51-like protein, complete cds. | |
| 700343818H1 | g218160 | 38 | −23 | gb105pln | Oryza sativa mRNA for elongation factor 1 beta′. | |
| 700345648H1 | g1488296 | 23 | −23 | gb105pln | Oryza sativa osRAD23 mRNA, complete cds. | |
| 700343890H1 | g1845196 | 50 | −72 | gb105pln | Z. mays mRNA for HMGc2 protein. | |
| 700342204H1 | g2331300 | 25 | −10 | gb105pln | Zea mays ribosomal protein S4 type I (rps4) mRNA, complete cds. | |
| 700346439H1 | g2408027 | 30 | −15 | gb105eukp | SPAC17G6.14c; atp-dependent rna helicase | |
| 700346074H1 | g1839580 | 52 | −10 | gb105pln | putative glycine-rich protein {clone CHEM 11} [Zea mays = maize, cv. INRA 258, | |
| | | | | | mercuric chloride-treated, leaves, mRNA Partial, 149 nt]. | |
| 700346916H1 | g2801536 | 9 | 5 | gb105eukp | LPL1; lysophospholipase homolog | |
| 700342872H1 | g1082146 | 11 | 5 | gb105allp | T18D3.3 | |
| 700346413H1 | g2501765 | 14 | −6 | gb105pln | Glycine max calmodulin-like domain protein kinase isoenzyme gamma mRNA, | |
| | | | | | complete cds. | |
| 700343761H1 | g2565339 | 42 | −30 | gb105pln | Lupinus luteus ribosomal protein S14 (rps14) mRNA, complete cds. | |
| 700346883H1 | g2262165 | 35 | −9 | gb105eukp | F5J6.10; predicted protein of unknown function | |
| 700345993H1 | g117368 | 68 | −47 | gb105pln | Z. mays mRNA for ribosomal protein L39. | |
| 700344745H1 | g895891 | 12 | −1 | gb105eukp | RPS5; ribosomal protein S5 | |
| 700347302H1 | g20163 | 28 | −24 | gb105pln | O. sativa Rr15 mRNA for 5S ribosomal RNA. | |
| 700343818H1 | g218340 | 38 | −23 | gb105pln | Triticum aestivum mRNA for elongation factor 1 beta′. | |
| 700347045H1 | g2529657 | 22 | 12 | gb105pln | Arabidopsis thaliana chromosome II BAC T30B22 genomic sequence, complete | |
| | | | | | sequence. | |
| 700346896H1 | g217973 | 24 | −23 | gb105pln | Zea mays gene for triosephosphate isomerase, complete cds. | |
| 700344853H1 | g2632928 | 8 | 5 | gb105allp | sorbitol dehydrogenase | |
| 700346013H1 | g443696 | 12 | 16 | gb105pln | Arabidopsis thaliana mRNA for ERD1 protein, complete cds. | |
| 700345221H1 | g434342 | 28 | −16 | gb105pln | A. thaliana (C24) mRNA for S18 ribosomal protein. | |
| 700343770H1 | g2330856 | 14 | 0 | gb105eukp | SPAC6F12.09; hypothetical protein | |
| 700343457H1 | g486229 | 19 | 1 | gb105eukp | APL2 | |
| 700346915H1 | g1688322 | 8 | 4 | gb105allp | PinA | |
| 700345169H1 | g20323 | 60 | −49 | gb105pln | O. sativa RAc1 gene for actin. | |
| 700342516H1 | g1486286 | 52 | −44 | gb105pln | C. sativus mRNA for T-complex polypeptide 1. | |
| 700379679H1 | g1841869 | 32 | −16 | gb105pln | Pimpinella brachycarpa elongation factor 1-beta (EF-1-beta) mRNA, complete cds. | |
| 700344178H1 | g1632830 | 29 | −2 | gb105pln | R. communis mRNA (unknown). | |
| 700344005H1 | g1181672 | 61 | −34 | gb105pln | Sorghum bicolor heat shock protein 70 cognate (hsc70) mRNA, partial cds. | |
| 700281809H2 | g397632 | 38 | −42 | gb105pln | T. aestivum translation initiation factor 4A. | |
| 700343085H1 | g557803 | 44 | −18 | gb105eukp | rir3 | |
| 700345848H1 | g22324 | 48 | −31 | gb105pln | Z. mays mRNA for H2B histone (clone cH2B221). | |
| 700346243H1 | g31419 | 25 | −9 | gb105allp | fibulin-1 C | |
| 700347384H1 | g22328 | 52 | −35 | gb105pln | Maize mRNA for a high mobility group protein. | |
| 700343809H1 | g1498385 | 56 | −70 | gb105pln | Zea mays actin (Maz87) gene, partial cds. | |
| 700380795H1 | g557828 | 21 | −12 | gb105eukp | sec11 | |
| 700344783H1 | g1813975 | 6 | 7 | gb105allp | cystathionine beta-synthase | |
| 700345826H1 | g902526 | 35 | −51 | gb105pln | Zea mays clone MubG7 ubiquitin fusion protein gene, complete cds. | |
| 700343920H1 | g287628 | 48 | −8 | gb105eukp | YS29A; ribosomal protein YS29 | |
| 700344323H1 | g22324 | 24 | −5 | gb105pln | Z. mays mRNA for H2B histone (clone CH2B221). | |
| 700343725H1 | g1262999 | 43 | −12 | gb105eukp | ZK287.5 | |
| 700342935H1 | g1628083 | 15 | −3 | gb105eukp | R07H5.5.8 | |
| 700343017H1 | g1885345 | 42 | −28 | gb105pln | T. aestivum mRNA for serpin WZS3. | |
| 700347227H1 | g643469 | 17 | −7 | gb105eukp | unknown | |
| 700344450H1 | g1546919 | 50 | 8 | gb105allp | translation initiation factor 5A | |
| 700345075H1 | g603403 | 12 | 6 | gb105eukp | YER163C; Yer163cp | |
| 700343381H1 | g474009 | 45 | −26 | gb105pln | Rice mRNA, partial homologous to ribosomal protein S19 gene. | |
| 700344613H1 | g414349 | 44 | −2 | gb105allp | ribosomal protein | |
| 700380650H1 | g168502 | 53 | −41 | gb105pln | Maize (Zea mays) histone H4 gene (H4C7), complete cds. | |
| 700345667H1 | g2345153 | 67 | −68 | gb105pln | Zea mays ribosomal protein S4 (rps4) mRNA, complete cds. | |
| 700345662H1 | g2511690 | 20 | −1 | gb105pln | Phaseolus vulgaris Moldavian encoding cystein proteinase precursor (clone cp41). | |
| 700346560H1 | g516306 | 12 | −3 | gb105eukp | Su(var)3-9; 51KDa protein | |
| 700346342H1 | g1063258 | 13 | 4 | gb105allp | p33/41 (annexin IV) | |
| 700343285H1 | g2564050 | 18 | 7 | gb105pln | Arabidopsis thaliana genomic DNA, chromosome 5, P1 clone: MUA22, complete | |
| | | | | | sequence. | |
| 700347252H1 | g1906825 | 59 | −51 | gb105pln | A. thaliana hsp81.2 gene. | |
| 700345472H1 | g18347 | 12 | 4 | gb105allp | glycine-rich protein | |
| 700343982H1 | g1055345 | 33 | 0 | gb105allp | beta-subunit of BK channels | |
| 700343691H1 | g2108344 | 30 | −14 | gb105pln | Brassica campestris small GTP-binding protein Bsar1a (bsar1a) mRNA, | |
| | | | | | complete cds. | |
| 700347002H1 | g168460 | 80 | −64 | gb105pln | Zea mays cyclophilin (CyP) mRNA, complete cds. | |
| 700346250H1 | g16117 | 20 | −22 | gb105pln | Avena sativa mRNA for protochlorophyllide reductase. | |
| 700344274H1 | g1620986 | 23 | −8 | gb105allp | 40S ribosomal protein S17 | |
| 700380406H1 | g168500 | 60 | −52 | gb105pln | Maize (Zea mays) histone H4 gene (H4C14), complete cds. | |
| 700343436H1 | g1872523 | 20 | −5 | gb105eukp | LSD1; negative regulator of cell death; zinc-finger protein Lsd1 | |
| 700345189H1 | g516042 | 13 | 14 | gb105pln | Arabidopsis thaliana Columbia Dwraf1 (DWF1) mRNA, complete cds. | |
| 700343815H1 | g2160155 | 18 | 6 | gb105pln | Sequence of BAC F21M12 from Arabidopsis thaliana chromosome 1, complete | |
| | | | | | sequence. | |
| 700281819H2 | g7357 | 19 | −3 | gb105eukp | ribosomal protein L7 (AA 1-246) | |
| 700343932H1 | g829147 | 71 | −85 | gb105pln | Z. mays gene for cyclophilin. | |
| 700344215H1 | g20250 | 47 | −19 | gb105pln | Oryza sativa H3 histone gene H3R-11. | |
| 700343691H1 | g1549221 | 27 | −11 | gb105pln | Tobacco mRNA for NtSar1 protein, complete cds. | |
| 700346069H1 | g214758 | 34 | −7 | gb105allp | ribosomal protein S22, 40S subunit | |
| 700343041H1 | g960356 | 35 | −31 | gb105pln | Barley mRNA for S-adenosylmethionine synthetase, complete cds. | |
| 700342543H1 | g2150130 | 13 | 3 | gb105eukp | cytoplasmic ribosomal protein S15a | |
| 700379874H1 | g556217 | 15 | 0 | gb105allp | spliceosomal protein | |
| 700347190H1 | g2326350 | 21 | −5 | gb105eukp | hypothetical protein | |
| 700345527H1 | g168500 | 71 | −58 | gb105pln | Maize (Zea mays) histone H4 gene (H4C14), complete cds. | |
| 700345273H1 | g2414643 | 27 | −8 | gb105pln | Yeast (Saccharomyces pombe) chromosome I cosmid c3H5. | |
| 700343038H1 | g1161251 | 32 | −23 | gb105pln | Glycine max nucleosome assembly protein 1 (SNAP-1) mRNA, complete cds. | |
| 700345943H1 | g2196445 | 12 | 4 | gb105allp | YSK1 | |
| 700344703H1 | g516684 | 54 | 7 | gb105allp | similar to bbc1 (breast basic conserved gene) of human | |
| 700342406H1 | g624220 | 46 | −30 | gb105pln | Oryza sativa signal recognition particle 19 kDa protein subunit SRP19 mRNA, | |
| | | | | | complete cds. | |
| 700379854H1 | g2618602 | 16 | 9 | gb105pln | Arabidopsis thaliana genomic DNA, chromosome 5, P1 clone: MSJ1, complete | |
| | | | | | sequence. | |
| 700346290H1 | g1906827 | 44 | −32 | gb105pln | A. thaliana hsp81.4 gene. | |
| 700346101H1 | g2773153 | 15 | −1 | gb105pln | Oryza sativa abscisic acid- and stress-inducible protein (Asr1) mRNA, | |
| | | | | | complete cds. | |
| 700342557H1 | g218176 | 30 | −44 | gb105pln | Rice mRNA for 21 kd polypeptide. | |
| 700344220H1 | g391604 | 32 | −32 | gb105pln | Arabidopsis thaliana mRNA for casein kinase II catalytic subunit. | |
| 700343590H1 | g303848 | 26 | −8 | gb105pln | Rice mRNA for nucleoside diphosphate kinase, complete cds. | |
| 700345888H1 | g2352494 | 16 | −4 | gb105eukp | TIR1; transport inhibitor response 1 | |
| 700380090H1 | g1054864 | 30 | −3 | gb105eukp | coxIII; cytochrome oxidase | |
| 700342583H1 | g1902991 | 56 | −45 | gb105pln | Rice mRNA for asparagine synthetase, complete cds. | |
| 700346939H1 | g499111 | 25 | −12 | gb105pln | Flaveria bidentis clone pNDK-b nucleoside diphosphate kinase mRNA, complete cds. | |
| 700346109H1 | g2529662 | 22 | −2 | gb105eukp | T30B22.5; putative small nuclear ribonucleoprotein, Sm D2 | |
| 700342234H1 | g1747310 | 18 | −3 | gb105eukp | AtCDC5; Myb-like DNA binding protein | |
| 700380552H1 | g182518 | 26 | 1 | gb105allp | ferritin light subunit | |
| 700347033H1 | g2302294 | 14 | 8 | gb105allp | unnamed protein product | |
| 700345489H1 | g434342 | 34 | −23 | gb105pln | A. thaliana (C24) mRNA for S18 ribosomal protein. | |
| 700379647H1 | g603764 | 69 | −38 | gb105allp | ionizing radiation resistance conferring protein | |
| 700346136H1 | g1403043 | 31 | −23 | gb105pln | H. chilense x T. turgidum conv. durum (Tritordeum) mRNA for S-adenosylmethionine | |
| | | | | | decarboxylase. | |
| 700342822H1 | g1167974 | 40 | −21 | gb105eukp | molecular chaperone Dd-TCP1 | |
| 700346975H1 | g565644 | 7 | 7 | gb105allp | hnRNP A2 protein | |
| 700281875H2 | g736271 | 46 | −42 | gb105pln | O. sativa hsp70 gene for heat shock protein 70. | |
| 700344292H1 | g398917 | 36 | −19 | gb105pln | B. napus cold induced protein (BnC24A) mRNA. | |
| 700343162H1 | g551319 | 27 | −11 | gb105pln | Yeast (Saccaromyces cerevisiae) chromosome VIII cosmid 9986. | |
| 700345344H1 | g473987 | 16 | −3 | gb105pln | Rice mRNA, partial homologous to histone H3 gene. | |
| 700344116H1 | g397395 | 65 | −50 | gb105pln | Z. mays MNB1b mRNA for DNA-binding protein. | |
| 700281846H2 | g1781299 | 33 | −11 | gb105eukp | splicing factor; transformer-SR ribonucleoprotein | |
| 700379908H1 | g2370231 | 48 | −40 | gb105pln | Hordum vulgare mRNA for putative acyl-CoA oxidase. | |
| 700345727H1 | g2618603 | 17 | −2 | gb105pln | Arabidopsis thaliana genomic DNA, chromosome 5, P1 clone: MSL3, complete | |
| | | | | | sequence. | |
| 700342303H1 | g168602 | 54 | −44 | gb105pln | Zea mays regulatory protein GF14-12 mRNA, complete cds. | |
| 700345707H1 | g485376 | 43 | −89 | gb105pln | Zea mays alpha-3-tubulin gene, complete cds. | |
| 700342769H1 | g2314600 | 8 | −0 | gb105allp | conserved hypthetical protein | |
| 700344273H1 | g992966 | 14 | −5 | gb105eukp | thioredoxin | |
| 700281838H2 | g2462076 | 26 | −13 | gb105pln | A. thaliana mRNA for oxa1-like gene. | |
| 700346720H1 | g971484 | 21 | −3 | gb105pln | L. esculentum mRNA for beta-galactosidase/galactanase protein (putative). | |
| 700344340H1 | g1809142 | 31 | 1 | gb105eukp | multicatalytic endopeptidase subunit C8; EC 3.4.99.46 | |
| 700346901H1 | g22180 | 86 | −83 | gb105pln | Maize gene for beta 1 tubulin. | |
| 700281887H2 | g2274990 | 51 | −55 | gb105pln | Hordeum vulgare mRNA for expressed sequence tag. | |
| 700343401H1 | g1785947 | 17 | −12 | gb105pln | Hordeum vulgare cytosolic triosephosphate somerase mRNA, complete cds. | |
| 700343266H1 | g984112 | 16 | 13 | gb105pln | C. annuum PCRTC mRNA. | |
| 700345847H1 | g2388943 | 26 | −10 | gb105eukp | SPAC27E2.03c; hypothetical protein | |
| 700346927H1 | g157564 | 20 | 2 | gb105eukp | Aats-glupro; transfer RNA-Glu-Pro aminoacyl synthetase | |
| 700346429H1 | g2661840 | 28 | −13 | gb105eukp | adk; adenosine kinase; EC 2.7.1.20 | |
| 700343728H1 | g2832762 | 20 | −5 | gb105eukp | EG:39E1.1 | |
| 700342766H1 | g1177368 | 100 | −11 | gb105pln | Z. mays mRNA for ribosomal protein L39. | |
| 700343213H1 | g1872546 | 19 | 7 | gb105allp | NIK | |
| 700342619H1 | g21794 | 31 | −40 | gb105pln | Wheat histone H4 gene. | |
| 700346106H1 | g1183936 | 16 | 2 | gb105pln | P. sativum 5S rRNA gene. | |
| 700379624H1 | g397395 | 75 | −58 | gb105pln | Z. mays MNB1b mRNA for DNA-binding protein. | |
| 700342403H1 | g2635371 | 8 | 1 | gb105allp | similar to hypothetical proteins | |
| 700380083H1 | g960290 | 33 | 1 | gb105pln | Ruta graveolens anthranilate synthase alpha-subunit mRNA, complete cds. | |
| 700346295H1 | g2760015 | 22 | −12 | gb105eukp | 60S ribosomal protein L14 | |
| 700346847H1 | g1914684 | 18 | −3 | gb105pln | D. carota mRNA for RAD23 protein, isoform II. | |
| 700346159H1 | g2668737 | 25 | −27 | gb105pln | Zea mays translation initiation factor 5A (TIF5A) mRNA, complete cds. | |
| 700346066H1 | g21229 | 43 | −32 | gb105pln | S. oleracea mRNA for stearoyl-acyl carrier protein desaturase. | |
| 700344052H1 | g1395190 | 43 | −38 | gb105pln | Spinacia oleracea L. mRNA for 26S proteasome ATPase subunit, complete cds. | |
| 700343820H1 | g1591042 | 10 | 4 | gb105allp | translation elongation factor EF-1, subunit alpha | |
| 700342893H1 | g2465426 | 12 | −2 | gb105eukp | JRG1.1.; 32 kDa protein | |
| 700346241H1 | g2842498 | 36 | −22 | gb105eukp | F21O9.240; protein kinase ADK1-subunit alpha | |
| 700346105H1 | g436920 | 14 | −1 | gb105eukp | lipoxygenase 1; EC 1.13.11.12 | |
| 700345320H1 | g563346 | 22 | −18 | gb105eukp | actin-like protein | |
| 700345841H1 | g443836 | 19 | −1 | gb105eukp | T07C4.9 | |
| 700342938H1 | g2191168 | 15 | −10 | gb105eukp | A_IG002P16.20 | |
| 700379790H1 | g710626 | 21 | 1 | gb105eukp | ERD15; ERD15 protein | |
| 700345439H1 | g172423 | 18 | 3 | gb105pln | Yeast (Saccharomyces cerevisiae) ribosomal protein L1 gene, complete cds. | |
| 700346921H1 | g170746 | 57 | −39 | gb105pln | Wheat histone H4 TH091 gene, complete cds. | |
| 700346691H1 | g303854 | 69 | −54 | gb105pln | Rice mRNA for ribosomal protein L7A, complete cds. | |
| 700346437H1 | g2852444 | 30 | −12 | gb105pln | Salix bakko mRNA for SUI1 homolog, complete cds. | |
| 700346342H1 | g189617 | 13 | 4 | gb105allp | protein PP4-X | |
| 700345887H1 | g218164 | 24 | −5 | gb105pln | Rice prepro-glutelin gene, complete cds. | |
| 700342493H1 | g386764 | 89 | −41 | gb105allp | hba2 alpha globin | |
| 700345983H1 | g1184771 | 68 | −71 | gb105pln | Zea mays glyceraldehyde-3-phosphate dehydrogenase GAPC2 (gpc2) mRNA, | |
| | | | | | complete cds. | |
| 700344302H1 | g22324 | 51 | −41 | gb105ln | Z. mays mRNA for H2B histone (clone cH2B221). | |
| 700379651H1 | g1209700 | 46 | −47 | gb105pln | Zea mays ribosomal protein L12 mRNA, complete cds. | |
| 700347091H1 | g498739 | 48 | −32 | gb105pln | H. vulgare (pMaW22) mRNA for beta-ketoacyl-ACP synthase. | |
| 700380188H1 | g633687: | 48 | −11 | gb105eukp | ubiquinol-cytochrome c reductase; EC 1.10.2.2 | |
| 700342858H1 | g20267 | 7 | 1 | gb105eukp | lipoxygenase L-2; EC 1.13.11.12 | |
| 700342896H1 | g1469218 | 39 | −13 | gb105pln | B. oleracea mRNA (unknown). | |
| 700346275H1 | g1575726 | 8 | 12 | gb105pln | Glycine max 14-3-3 related protein SGF14B mRNA, partial cds. | |
| 700343322H1 | g22171 | 19 | 13 | gb105pln | Maize nuclear ARS3 DNA autonomously replicating in Yeast. | |
| 700380159H1 | g22483 | 39 | −40 | gb105pln | Z. mays RNA for superoxide dismutase Sod4. | |
| 700343937H1 | g532572 | 15 | −3 | gb105eukp | LoxA; lipoxygenase 1; EC 1.13.11.12; linoleate:oxygen oxioreductase, lipoxidase, | |
| | | | | | carotene oxidase | |
| 700343079H1 | g532821 | 8 | 7 | gb105eukp | F57B9.10 | |
| 700345325H1 | g683502 | 19 | −5 | gb105eukp | protein phosphatase 2A 65 kDa regulatory subunit | |
| 700379811H1 | g2465018 | 40 | −35 | gb105pln | Fragaria vesca partial mRNA for putative cystathionine gamma synthase. | |
| 700346790H1 | g1006830 | 21 | −1 | gb105pln | Gossypium hirsutum acyl-CoA-binding protein mRNA, complete cds. | |
| 700342826H1 | g2274990 | 61 | −49 | gb105pln | Hordeum vulgare mRNA for expressed sequence tag. | |
| 700344990H1 | g2653284 | 40 | −38 | gb105pln | Oryza sativa mRNA for enoyl-ACP reductase. | |
| 700343013H1 | g1183936 | 15 | 3 | gb105pln | P. sativum 5S rRNA gene. | |
| 700343586H1 | g1167527 | 14 | −17 | gb105eukp | Cytb; cytochrome b | |
| 700343281H1 | g2370518 | 5 | 6 | gb105allp | ribosomal protein | |
| 700343308H1 | g498642 | 48 | −24 | gb105pln | Zea mays G-box binding factor 1 (GBF1) mRNA, complete cds. | |
| 700379982H1 | g1167963 | 19 | 6 | gb105eukp | 18-56 protein | |
| 700344703H1 | g510552 | 54 | 7 | gb105allp | ribosomal protein L13 | |
| 700344193H1 | g1523921 | 17 | 1 | gb105eukp | F27D4.1 | |
| 700343076H1 | g2330709 | 6 | 8 | gb105eukp | SPAC17H9.02; hyothetical helicase | |
| 700344804H1 | g2570506 | 42 | −30 | gb105pln | Oryza sativa ribosomal protein mRNA, complete cds. | |
| 700342928H1 | g473602 | 45 | −43 | gb105pln | Zea mays W-22 histone H2A mRNA, complete cds. | |
| 700345822H1 | g1218041 | 18 | −14 | gb105pln | Arabidopsis thaliana secreted purple acid phosphatase precursor (PAP1) gene, | |
| | | | | | complete cds. | |
| 700344120H1 | g1488297 | 17 | 8 | gb105allp | osRAD23 | |
| 700343863H1 | g1213072 | 42 | −2 | gb105pln | O. sativa (Tainong 67) mRNA for heat shock protein 17.8. | |
| 700347067H1 | g1655679 | 27 | −13 | gb105eukp | 3-hydroxy-3-methylglutaryl-CoA-synthase | |
| 700346540H1 | g1903019 | 61 | −31 | gb105eukp | curly leaf | |
| 700343332H1 | g347715 | 16 | −0 | gb105eukp | shows 46% identity to human placental protein 15 (PP15) | |
| 700379754H1 | g218176 | 30 | 8 | gb105pln | Rice mRNA for 21kd polypeptide. | |
| 700344801H1 | g2668747 | 29 | 9 | gb105pln | Zea mays ribosomal protein L17 (rp117) mRNA, complete cds. | |
| 700344880H1 | g915193 | 34 | −34 | gb105pln | Chlamydomonas reinhardtii mRNA for actin, complete cds. | |
| 700342711H1 | g1632830 | 28 | −11 | gb105pln | R. communis mRNA (unknown). | |
| 700379647H1 | g8720565 | 12 | 1 | gb105eukp | C14A4.2 | |
| 700347215H1 | g166606 | 28 | −17 | gb105eukp | anthranilate synthase alpha subunit | |
| 700379739H1 | g576998 | 8 | 7 | gb105eukp | C08B11.5 | |
| 700343280H1 | g145301 | 11 | 2 | gb105allp | adk ORF | |
| 700346001H1 | g1321660 | 41 | −36 | gb105pln | Rice mRNA for ascorbate peroxidase, complete cds. | |
| 700343413H1 | g397340 | 8 | 4 | gb105allp | DNA-binding protein | |
| 700343933H1 | g22484 | 50 | −43 | gb105pln | Z. mays RNA for superoxide dismutase Sod4A. | |
| 700343250h1 | g2570223 | 19 | −15 | gb105pln | Sequence of BAC F20D22 from Arabidopsis thaliana chromosome 1, complete | |
| | | | | | sequence. | |
| 700344907H1 | g473604 | 26 | −29 | gb105pln | Zea mays W-22 histone H2B mRNA, complete cds. | |
| 700344149H1 | g2814799 | 18 | −1 | gb105eukp | ZK858.6 | |
| 700379931H1 | g2114206 | 22 | −35 | gb105pln | Rice DNA for glutaredoxin, complete cds. | |
| 700380220H1 | g558651 | 27 | −12 | gb105pln | T. aestivum VDAC3 mRNA for voltage dependent anion channel. | |
| 700346518H1 | g1944202 | 32 | −36 | gb105pln | Perilla frutescens mRNA for actin, partial cds. | |
| 700344120H1 | g2264320 | 18 | 12 | gb105pln | Arabidopsis thaliana genomic DNA, chromosome 5, P1 clone: MX110, complete | |
| | | | | | sequence. | |
| 700346601H1 | g624675 | 35 | −26 | gb105pln | Citrus maxima citrate synthase (cit) mRNA, nuclear gene encoding mitochondrial | |
| | | | | | protein, complete cds. | |
| 700342514H1 | g2464911 | 23 | 3 | gb105eukp | splicing factor homolog | |
| 700342564H1 | g469247 | 21 | 0 | gb105pln | Helianthus annuuus ribosomal protein S3a mRNA, complete cds. | |
| 700343173H1 | g438279 | 24 | −9 | gb105allp | Ribosomal protein L7 | |
| 700347280H1 | g556672 | 50 | −45 | gb105pln | S. cereale (Halo) chloroplast mRNA for heat-shock protein. | |
| 700342365H1 | g1066474 | 14 | −6 | gb105eukp | YPR118W; Ypr118wp | |
| 700342946H1 | g166857 | 40 | −27 | gb105pln | Arabidopsis thaliana cytoplasmic ribosomal protein mRNA, complete cds. | |
| 700380278H1 | g1911165 | 38 | −30 | gb105pln | S. tuberosum mRNA for soluble-starch-synthase. | |
| 700380469H1 | g337187 | 18 | −0 | gb105allp | cytochrome oxidase subunit II | |
| 700344051H1 | g2196466 | 14 | 5 | gb105allp | TATA binding protein-associated factor | |
| 700343834H1 | g293888 | 29 | −11 | gb105pln | Zea mays, glyceraldehyde-3-phosphate dehydrogenase mRNA, 3′ end (clone GAPC2). | |
| 700343366H1 | g1261930 | 8 | 6 | gb105allp | unknown | |
| 700346763H1 | g829147 | 91 | −82 | gb105pln | Z. mays gene for cyclophilin. | |
| 700379655H1 | g2407799 | 20 | −15 | gb105pln | Arabidopsis thaliana mRNA for histone H2A.F/Z. | |
| 700347065H1 | g2656029 | 16 | −17 | gb105pln | Arabidopsis thaliana genomic DNA, chromosome 5, P1 clone: MOB2. | |
| 700346892H1 | g459894 | 54 | −63 | gb105pln | Zea mays sus1 gene, complete cds. | |
| 700344720H1 | g1302342 | 6 | 6 | gb105eukp | ORF YNL275w | |
| 700342296H1 | g1468961 | 23 | −1 | gb105allp | ribophorin | |
| 700345121H1 | g2302292 | 18 | 2 | gb105allp | unnamed protein product | |
| 700345431H1 | g2677823 | 9 | 9 | gb105pln | Prunus armeniaca abscisic stress ripening protein homolog mRNA, complete cds. | |
| 700345443H1 | g541654 | 14 | 7 | gb105eukp | cdc2rk; Dcdrk kinase | |
| 700345652H1 | g204477 | 59 | 1 | gb105allp | Rat submandibular gland secretory protein | |
| 700345413H1 | g602605 | 71 | −70 | gb105pln | Zea mays tandem genes for alpha1-tubulin and alpha2-tubulin. | |
| 700343677H1 | g21692 | 28 | −13 | gb105pln | T. aestivum mRNA 1 for cathepsin B (2529). | |
| 700342328H1 | g551289 | 48 | −68 | gb105pln | Z. mays (W22) phosphoglycerate mutase gene exons 2-8. | |
| 700347028H1 | g2738247 | 31 | −19 | gb105pln | Arabidopsis thaliana cobalamin-independent methionine synthase (ATCIMS) mRNA, | |
| | | | | | complete cds. | |
| 700346607H1 | g1322867 | 8 | 7 | gb105allp | ORF YGL220w | |
| 700347072H1 | g1381150 | 17 | 5 | gb105allp | Hs-CUL-4B | |
| 700345462H1 | g287834 | 32 | −18 | gb105pln | Z. mays yptm2 cDNA. | |
| 700344227H1 | g2827142 | 17 | −12 | gb105pln | Arabidopsis thaliana cellulose synthase catalytic subunit (Ath-B) mRNA, | |
| | | | | | complete cds. | |
| 700346005H1 | g190234 | 28 | 3 | gb105allp | acidic ribosomal phosphoprotein (P1) | |
| 700343996H1 | g2809480 | 65 | −63 | gb105pln | Oryza sativa calmodulin (CaM2) mRNA, complete cds. | |
| 700342328H1 | g168587 | 92 | −76 | gb105pln | Zea mays cofactor-independent phosphoglycerate mutase mRNA, complete cds. | |
| 700343158H1 | g2739020 | 19 | 15 | gb105pln | Rhododendron metternichii var. hondoense CT, AT, GT repeat, genomic sequence. | |
| 700343485H1 | g736271 | 51 | −26 | gb105pln | O. sativa hsp70 gene for heat shock protein 70. | |
| 700346269H1 | g1408515 | 5 | 7 | gb105eukp | ribosomal protein L22 homolog | |
| 700281822H2 | g871515 | 71 | 5 | gb105allp | Psst70 (stress 70 protein) | |
| 700343144H1 | g1575129 | 96 | −59 | gb105pln | Zea mays lumenal binding protein cBiPe3 mRNA, complete cds. | |
| 700347045H1 | g303854 | 64 | −25 | gb105pln | Rice mRNA for ribosomal protein L7A, complete cds. | |
| 700345058H1 | g1532051 | 33 | −24 | gb105pln | P. argentatum mRNA for farnesyl diphosphate synthase (FPS2). | |
| 700344941H1 | g575730 | 33 | −34 | gb105pln | Z. mays mRNA for transmembrane protein. | |
| 700345472H1 | g2226370 | 10 | 4 | gb105eukp | RNA-binding protein | |
| 700343377H1 | g1015315 | 23 | 5 | gb105pln | Pisum sativum (clone PsRCI35-2) ribosomal protein L41 mRNA, complete cds. | |
| 700380747H1 | g556685 | 64 | −51 | gb105pln | Z. mays mRNA for ADP-ribosylation factor. | |
| 700342333H1 | g736271 | 31 | −10 | gb105pln | O. sativa hsp70 gene for heat shock protein 70. | |
| 700342734H1 | g218160 | 47 | −47 | gb105pln | Oryza sativa mRNA for elongation factor 1 beta′. | |
| 700380283H1 | g168492 | 61 | −48 | gb105pln | Corn histone H3 (H3C3) gene, complete cds. | |
| 700345759H1 | g2662342 | 83 | −76 | gb105pln | Oryza sativa mRNA for EF-1 alpha, complete cds. | |
| 700344835H1 | g397395 | 69 | −68 | gb105pln | Z. mays MNB1b mRNA for DNA-binding protein. | |
| 700344141H1 | g2224910 | 36 | −27 | gb105pln | Daucus carota somatic embryogenesis receptor-like kinase mRNA, complete cds. | |
| 700343702H1 | g1088348 | 8 | 5 | gb105allp | adenylate kinase | |
| 700281812H2 | g398848 | 84 | −56 | gb105pln | Z. mays mRNA for beta 5 tubulin. | |
| 700344450H1 | g2668737 | 59 | 2 | gb105pln | Zea mays translation initiation factor 5A (TIF5A) mRNA, complete cds. | |
| 700344215H1 | g168494 | 47 | −18 | gb105pln | Maize (Zea mays) histone H3 gene (H3C2), complete cds. | |
| 700344102H1 | g1256595 | 40 | −17 | gb105allp | LytB | |
| 700346953H1 | g2739218 | 47 | −33 | gb105pln | Hordeum vulgare S28 ribosomal protein. | |
| 700342896H1 | g2584827 | 36 | −8 | gb105pln | Sequence of BAC F12F1 from Arabidopsis thaliana chromosome 1, complete | |
| | | | | | sequence. | |
| 700347061H1 | g2384757 | 54 | −47 | gb105pln | Oryza sativa GDP dissociation inhibitor protein OsGDI1 (OsGDI1) mRNA, | |
| | | | | | complete cds. | |
| 700343717H1 | g2668743 | 37 | −38 | gb105pln | Zea mays ubiquitin conjugating enzyme (UBC) mRNA, complete cds. | |
| 700343011H1 | g2760168 | 12 | 13 | gb105pln | Arabidopsis thaliana genomic DNA, chromosome 5, P1 clone: MEE6, complete | |
| | | | | | sequence. | |
| 700346901H1 | g416144 | 70 | −69 | gb105pln | Zea mays beta-4 tubulin (tub4) mRNA, complete cds. | |
| 700380603H1 | g2656016 | 7 | 6 | gb105allp | cell-cycle regulatory protein | |
| 700343517H1 | g2642324 | 12 | 6 | gb105eukp | PRO4; G-actin binding protein; profilin | |
| 700379831H1 | g499011 | 67 | −61 | gb105pln | S. vulgare SoAc1 mRNA. | |
| 700379677H1 | g22314 | 61 | −82 | gb105pln | Maize mRNA for GSH gluthathione S-transferase I (GST; EC 2.5.1.18). | |
| 700347334H1 | g2463332 | 23 | −22 | gb105pln | Oryza sativa mRNA for M79 protein. | |
| 700343933H1 | g1574937 | 29 | −62 | gb105pln | Zea mays superoxide dismutase 4 (sod4) gene, partial cds. | |
| 700346827H1 | g285638 | 23 | 6 | gb105eukp | inorganic pyrophosphatase ase′; EC 3.6.1.1 | |
| 700343483H1 | g473169 | 48 | −7 | gb105eukp | subunit of the mitochondrial pyruvate dehydrogenase complex; E1 alpha subunit of | |
| | | | | | pyruvate dehydrogenase precursor; EC 1.2.4.1 | |
| 700345248H1 | g157535 | 10 | 5 | gb105eukp | gol; zinc finger protein; goliath protein | |
| 700345912H1 | g643073 | 22 | −2 | gb105pln | Fragaria x ananassa putative 40S ribosomal protein s12 mRNA, complete cds. | |
| 700343433H1 | g564024 | 34 | −10 | gb105eukp | ZM/DHLAAT; dihydrolipoamide acetyl transferase | |
| 700380575H1 | g2558659 | 14 | 1 | gb105allp | No definition line found | |
| 700346313H1 | g57004 | 8 | 6 | gb105allp | pyrimidine binding protein 2 | |
| 700345237H1 | g836764 | 15 | 3 | gb105eukp | YFR009W | |
| 700344542H1 | g2076623 | 10 | 5 | gb105allp | PtrB | |
| 700346145H1 | g4026 | 23 | −2 | gb105eukp | NAM8 | |
| 700345875H1 | g2558943 | 50 | −36 | gb105pln | Gossypium hirsutum histone 3 mRNA, complete cds. | |
| 700344166H1 | g1008296 | 20 | −3 | gb105pln | Yeast (Saccharomyces cerevisiae) chromosome X reading frame ORF YJL111w. | |
| 700342546H1 | g1006731 | 18 | −13 | gb105eukp | CCT3 | |
| 700379675H1 | g309071 | 14 | −7 | gb105eukp | RPS7; ribosomal protein S7 | |
| 700346257H1 | g633109 | 14 | 8 | gb105pln | Rice mRNA for plasma membrane H+-ATPase, complete cds. | |
| 700342977H1 | g416251 | 46 | −36 | gb105pln | Rice mRNA for acetohydroxy acid reductoisomerase, partial sequence. | |
| 700343930H1 | g20163 | 44 | −32 | gb105pln | O. sativa Rr15 mRNA for 5S ribosomal RNA. | |
| 700343318H1 | g536190 | 19 | −3 | gb105eukp | COQ1 | |
| 700346147H1 | g2414643 | 24 | −0 | gb105pln | Yeast (Saccharomyces pombe) chromosome I cosmid C3H5. | |
| 700344913H1 | g2511530 | 82 | −71 | gb105pln | Eleusine indica alpha tubulin 1 (TUA1) mRNA, complete cds. | |
| 700343852H1 | g304674 | 11 | −1 | gb105eukp | Eif2<alpha>; translation initiation; eIF-2 alpha-subunit | |
| 700344014H1 | g17780 | 55 | −2 | gb105eukp | 3-oxoacyl-[acyl-carrier protein] reductase; EC 1.1.1.100 | |
| 700342780H1 | g1041336 | 31 | −12 | gb105eukp | F54D5.8 | |
| 700380683H1 | g525196 | 6 | 8 | gb105allp | epsilon-COP; 1d1Fp | |
| 700346495H1 | g899609 | 40 | −32 | gb105pln | Zea mays acidic ribosomal protein P2 (RPA-2A1) mRNA, complete cds. | |
| 70034588H1 | g17928 | 39 | −32 | gb105pln | B. rapa cv.R500 mRNA for stearoyl-acyl carrier protein desaturase. | |
| 700346118H1 | g487934 | 15 | 3 | gb105eukp | NAM8; Nam8p: Putative RNA binding proteins | |
| 700343968H1 | g2245377 | 24 | −10 | gb105pln | Arabidopsis thaliana auxin response factor 1 (ARF1) mRNA, complete cds. | |
| 700342789H1 | g22322 | 21 | −32 | gb105pln | Z. mays mRNA for H2B histone (clone cH2B214). | |
| 700342657H1 | g2415704 | 22 | 4 | gb105allp | proline iminopeptidase | |
| 700345820H1 | g2257755 | 52 | −39 | gb105pln | Zea mays nucleolar histone deacetylase HD2-p39 mRNA, complete cds. | |
| 700345476H1 | g1246843 | 22 | −6 | gb105eukp | G2834; ribosomal protein | |
| 700345288H1 | g22447 | 26 | 3 | gb105pln | Zea mays ZMPMS2 gene for 19 kDa zein protein. | |
| 700344810H1 | g2369713 | 54 | −45 | gb105pln | Beta vulgaris cDNA for elongation factor 2. | |
| 700344322H1 | g166410 | 33 | −6 | gb105eukp | nucleic acid binding protein; Alfin-1 | |
| 700380062H1 | g168500 | 41 | −10 | gb105pln | Maize (Zea mays) histone H4 gene (H4C14), complete cds. | |
| 700345975H1 | g436032 | 61 | −4 | gb105eukp | 60S ribosomal protein L34 | |
| 700344038H1 | g200781 | 20 | 5 | gb105allp | ribosomal protein L32-3A gene | |
| 700346602H1 | g536891 | 65 | −56 | gb105pln | Wheat mRNA for protein H2A, complete cds, clone wcH2A-4. | |
| 700344146H1 | g1064826 | 7 | 3 | gb105allp | Actin | |
| 700343255H1 | g2827716 | 16 | 5 | gb105eukp | F6H11.180; predicted protein | |
| 700347246H1 | g1235520 | 14 | 0 | gb105allp | orf gene product | |
| 700346584H1 | g1209756 | 44 | −9 | gb105eukp | putative sugar transporter; integral membrane protein | |
| 700343407H1 | g529009 | 22 | −5 | gb105eukp | PFK; PPi-dependent phosphofructo-1-kinase; EC 2.7.1.90 | |
| 700380182H1 | g1370455 | 16 | −5 | gb105pln | Yeast (Saccharomyces cerevisiase) chromosome XVI reading ORF YPL220w. | |
| 700346002H1 | g498737 | 50 | −52 | gb105pln | H. vulgare (pMaW21) pseudo mRNA for beta-ketoacyl-ACP synthase (partial). | |
| 700342333H1 | g22342 | 69 | −57 | gb105pln | Maize gene for heat shock protein 70 exon 2 and 3′-UT (hsp70; clone pMON 9502). | |
| 700343276H1 | g551319 | 28 | −14 | gb105pln | Yeast (Saccharomyces cerevisiae) chromosome VIII cosmid 9986. | |
| 700347024H1 | g264277 | 82 | −69 | gb105pln | auxin-binding protein [corn, mRNA, 795 nt]. | |
| 700345724H1 | g450879 | 60 | −63 | gb105pln | A. thaliana hsc70-1 gene. | |
| 700344548H1 | g2351064 | 14 | 15 | gb105pln | Arabidopsis thaliana genomic DNA, chromosome 5, P1 clone: MDJ22. | |
| 700342760H1 | g303731 | 34 | −16 | gb105pln | Pea mRNA for GTP-binding protein, complete cds. | |
| 700347216H1 | g2511572 | 28 | −5 | gb105eukp | prcga; multicatalytic endopeptidase; EC 3.4.99.46 | |
| 700344868H1 | g22314 | 48 | −80 | gb105pln | Maize mRNA for GSH gluthathione S-transferase I (GST; EC 2.5.1.18). | |
| 700343138H1 | g1568483 | 15 | 2 | gb105pln | B. vulgaris mRNA for cyclin. | |
| 700346532H1 | g1263159 | 13 | 15 | gb105pln | O. sativa DNA for LRK2 gene. | |
| 700343852H1 | g2706460 | 17 | −5 | gb105eukp | SPAC3G9.09c; hypothetical translational initiation factor 2 alpha | |
| 700345142H1 | g1006624 | 15 | −1 | gb105eukp | alt A45; proteindisulfidisomerase | |
| 700342461H1 | g1652223 | 12 | 1 | gb105allp | ABC1-like | |
| 700342754H1 | g1519248 | 43 | −28 | gb105pln | Oryza sativa GF14-b protein mRNA, complete cds. | |
| 700379947H1 | g2244847 | 8 | 7 | gb105eukp | hydroxyproline-rich glycoprotein homolog | |
| 700344805H1 | g550543 | 37 | −21 | gb105pln | A. thaliana mRNA for ribosomal protein L16. | |
| 700346859H1 | g1203905 | 21 | −4 | gb105eukp | M(1)15D; M(1)15D | |
| 700344374H1 | g1575127 | 57 | −55 | gb105pln | Zea mays lumenal binding protein cBiPe2 mRNA, complete cds. | |
| 700342284H1 | g156374 | 19 | −5 | gb105eukp | mlc-2 myosin light chain 2 | |
| 700342573H1 | g2765423 | 54 | −37 | gb105allp | immunoglobulin kappa light chain | |
| 700343119H1 | g841308 | 24 | 6 | gb105allp | TAFII32 precursor | |
| 700379649H1 | g790507 | 65 | −55 | gb105pln | Z. mays mRNA for 60S acidic ribosomal protein. | |
| 700346920H1 | g1430978 | 22 | −13 | gb105pln | Yeast (Saccharomyces cerevisiae) chromosome IV reading frame ORF YDL014w. | |
| 700345761H1 | g2827140 | 50 | −15 | gb105pln | Arabidopsis thaliana cellulose synthase catalytic subunit (Ath-A) mRNA, | |
| | | | | | complete cds. | |
| 700345346H1 | g2789434 | 10 | 8 | gb105allp | CLB1 | |
| 700380172H1 | g1008401 | 29 | −14 | gb105pln | Yeast (Saccharomyces cerevisiae) chromosome X reading frame ORF YJL191w. | |
| 700344340H1 | g2285802 | 34 | −1 | gb105eukp | 26S proteasome alpha subunit | |
| 700342258H1 | g6815 | 28 | −16 | gb105eukp | R08D7.3 | |
| 700342466H1 | g2225941 | 35 | −9 | gb105allp | Mdes protein | |
| 700346422H1 | g1322276 | 21 | −0 | gb105pln | Triticum aestivum histone H2A gene, complete cds. | |
| 700344925H1 | g22155 | 93 | −76 | gb105pln | Z. mays mRNA for alpha-tubulin 5. | |
| 700380233H1 | g444022 | 37 | −30 | gb105pln | N. tabacum mRNA for pyruvate kinase. | |
| 700347071H1 | g829147 | 76 | −68 | gb105pln | Z. mays gene for cyclophilin. | |
| 700344931H1 | g20250 | 58 | −44 | gb105pln | Oryza sativa H3 histone gene H3R-11. | |
| 700343627H1 | g395078 | 25 | 9 | gb105pln | B. rapa ubiquitin and ribosomal protein mRNA, complete CDS's. | |
| 700346185H1 | g762785 | 42 | −1 | gb105eukp | cysteine proteinase inhibitor | |
| 700346213H1 | g868002 | 33 | −19 | gb105pln | Pumpkin mRNA for aconitase, complete cds. | |
| 700347150H1 | g914875 | 27 | −7 | gb105eukp | unknown | |
| 700344111H1 | g2190547 | 51 | −21 | gb105eukp | F5I14.13 | |
| 700345736H1 | g303852 | 65 | −67 | gb105pln | Rice mRNA for ribosomal protein L3, complete cds. | |
| 700345108H1 | g454913 | 36 | −26 | gb105pln | A. porrum LDJ2 mRNA. | |
| 700345661H1 | g455419 | 17 | 1 | gb105allp | PR264/SC35 | |
| 700342473H1 | g2564046 | 26 | −6 | gb105pln | Arabidopsis thaliana genomic DNA, chromosome 5, P1 clone: MGI19, complete | |
| | | | | | sequence. | |
| 700380212H1 | g21794 | 33 | −39 | gb105pln | Wheat histone H4 gene. | |
| 700342780H1 | g1707657 | 36 | −17 | gb105eukp | DnaJ homologue | |
| 700342686H1 | g2341028 | 40 | −4 | gb105eukp | F19P19.5 | |
| 700343395H1 | g498739 | 44 | −26 | gb105pln | H. vulgare (pMaW22) mRNA for beta-ketoacyl-ACP synthase. | |
| 700345336H1 | g2281104 | 8 | 3 | gb105eukp | F18O19.23; Glucan endo-1,3-beta glucosidase isolog | |
| 700281817H2 | g44204 | 8 | 2 | gb105allp | recA protein | |
| 700342324H1 | g1388021 | 13 | 7 | gb105allp | UDP-glucose pyrophosphorylase | |
| 700344050H1 | g22187 | 38 | −31 | gb105pln | Z. mays mRNA for b-32 protein, putative regulatory factor of zein expression | |
| | | | | | (clone b-32.129). | |
| 700343032H1 | g1498052 | 76 | −74 | gb105pln | Zea mays ribosomal protein S8 mRNA, complete cds. | |
| 700380284H1 | g398607 | 27 | −9 | gb105pln | A. thaliana mRNA for elongation factor 1 beta. | |
| 700347151H1 | g2760167 | 22 | −0 | gb105pln | Arabidopsis thaliana genomic DNA, chromosome 5, P1 clone: MCO15, complete | |
| | | | | | sequence. | |
| 700342504H1 | g2832685 | 21 | −1 | gb105eukp | T10I14.180; putative protein | |
| 700347360H1 | g2343181 | 20 | −10 | gb105eukp | MDL3; catalyzes dissociation of (R)-mandelonitrile to hydrogen cyanide and | |
| | | | | | benzaldehyde; (R)-(+)-madelonitrile lyase isoform MDL3 precursor; EC 4.1.2.10 | |
| 700344961H1 | g2245074 | 29 | −4 | gb105eukp | hypothetical protein | |
| 700346736H1 | g2827314 | 24 | 3 | gb105eukp | casein kinase | |
| 700379891H1 | g1053056 | 20 | −31 | gb105pln | Triticum aestivum histone H3 gene, partial cds, clone W1. | |
| 700344091H1 | g2293565 | 33 | −13 | gb105pln | Oryza sativa ADP-ribosylation factor 1 (Os-ARF1) mRNA, complete cds. | |
| 700342280H1 | g20237 | 43 | −42 | gb105pln | O. sativa (rice) constitutive GOS2 gene. | |
| 700345519H1 | g2196671 | 85 | −72 | gb105pln | Z. mays mRNA for HMG protein. | |
| 700342268H1 | g762850 | 10 | 4 | gb105allp | p21 protein | |
| 700344751H1 | g736271 | 17 | −41 | gb105pln | O. sativa hsp70 gene for heat shock protein 70. | |
| 700347259H1 | g2599091 | 44 | −32 | gb105pln | Arabidopsis thaliana WD-40 repeat protein MSI4 (MSI4) mRNA, complete cds. | |
| 700342532H1 | g522131 | 10 | 5 | gb105eukp | D2045.8 | |
| 700344705H1 | g1488265 | 19 | −7 | gb105eukp | high affinity calcium antiporter CAX1 | |
| 700342480H1 | g168539 | 58 | −15 | gb105pln | Zea mays putative ribosomal protein S22 homolog mRNA, partial cds. | |
| 700345577H1 | g825783 | 22 | 7 | gb105pln | Nicotiana tabacum ribosomal protein L41 mRNA, complete cds. | |
| 700345926H1 | g431155 | 19 | −3 | gb105pln | Lily mRNA for ORF, partial cds. | |
| 700342916H1 | g168639 | 58 | −82 | gb105pln | Zea mays autonomous transposable element En-1 mosaic protein gene, complete cds. | |
| 700379877H1 | g2257484 | 8 | 0 | gb105eukp | pi04; HYPOTHETICAL 45.0 KD PROTEIN IN NOT1/CDC39-HMR INTERGENIC | |
| | | | | | REGION | |
| 700347208H1 | g1808578 | 12 | −0 | gb105allp | proteasome subunit p112 | |
| 700346001H1 | g2274983 | 43 | −25 | gb105pln | Hordeum vulgare mRNA for expressed sequence tag. | |
| 700344988H1 | g2827530 | 16 | −5 | gb105eukp | F8F16.170; predicted protein | |
| 700345631H1 | g387509 | 17 | 4 | gb105allp | protein disulfide isomerase | |
| 700345323H1 | g2282583 | 82 | −76 | gb105pln | Zea mays elongation factor 1-alpha (EF1-A) mRNA, complete cds. | |
| 700344184H1 | g2529657 | 16 | 13 | gb105pln | Arabidopsis thaliana chromosome II BAC T30B22 genomic sequence, complete | |
| | | | | | sequence. | |
| 700380133H1 | g473604 | 20 | −35 | gb105pln | Zea mays W-22 histone H2B mRNA, complete cds. | |
| 700344508H1 | g2145356 | 8 | −3 | gb105eukp | ATHB-14; HD-Zip protein | |
| 700343514H1 | g600710 | 17 | −19 | gb105eukp | SAR1 | |
| 700346885H1 | g2564233 | 27 | −2 | gb105eukp | Ci-zeta; proteasome Z subunit | |
| 700344253H1 | g2827142 | 54 | −46 | gb105pln | Arabidopsis thaliana cellulose synthase catalytic subunit (Ath-B) mRNA, | |
| | | | | | complete cds. | |
| 700345768H1 | g2463564 | 45 | −27 | gb105pln | Oryza sativa mRNA for squalene synthase, complete cds. | |
| 700344872H1 | g886470 | 44 | −48 | gb105pln | C. roseus MetE mRNA for methionine synthase. | |
| 700342686H1 | g310933 | 37 | −4 | gb105eukp | r117; a subunit of 60S ribosomal protein; 60S ribosomal protein subunit L17 | |
| 700344815H1 | g1171578 | 25 | −12 | gb105pln | N. tabacum mRNA for HSR515 protein. | |
| 700343020H1 | g2313520 | 13 | −9 | gb105allp | cyclopropane fatty acid synthase (cfa) | |
| 700380209H1 | g486151 | 9 | 6 | gb105eukp | ORF YKL056c | |
| 700379630H1 | g2257755 | 27 | −31 | gb105pln | Zea mays nucleolar histone deacetylase HD2-p39 mRNA, complete cds. | |
| 700347048H1 | g1184773 | 59 | −10 | gb105pln | Zea mays glyceraldehyde-3-phosphate dehydrogenase GAPC3 (gpc3) mRNA, | |
| | | | | | complete cds. | |
| 700345243H1 | g303856 | 25 | −19 | gb105pln | Rice mRNA for ubiquitin protein fused to a ribosomal protein, complete cds. | |
| 700344691H1 | g624956 | 31 | −15 | gb105pln | C. reinhardtii mRNA for ribosomal protein S18. | |
| 700343266H1 | g2429342 | 15 | 16 | gb105pln | Brassica napus calreticulin mRNA, complete cds. | |
| 700380214H1 | g1335861 | 50 | −45 | gb105pln | Glycine max clathrin heavy chain mRNA, complete cds. | |
| 700347368H1 | g550019 | 8 | 6 | gb105allp | ribosomal protein L28 | |
| 700347285H1 | g2388689 | 19 | 2 | gb105allp | GH1 protein | |
| 700346181H1 | g168500 | 45 | 14 | gb105pln | Maize (Zea mays) histone H4 gene (H4C14), complete cds. | |
| 700344447H1 | g786132 | 14 | −13 | gb105eukp | RCc3 | |
| 700343457H1 | g520884 | 19 | 1 | gb105eukp | APL2; clathrin assembly; Ap12 | |
| 700345511H1 | g601790 | 23 | −1 | gb105pln | Arabidopsis thaliana protein kinase (AFC3) mRNA, partial cds. | |
| 700343070H1 | g666053 | 46 | −52 | gb105pln | H. vulgare mRNA for photosysteme I antenna protein. | |
| 700281886H2 | g2618599 | 14 | −10 | gb105pln | Arabidopsis thaliana genomic DNA, chromosome 5, P1 clone: MBD2, | |
| | | | | | complete sequence. | |
| 700343796H1 | g1523950 | 12 | 1 | gb105eukp | F54F3.4 | |
| 700345905H1 | g436031 | 23 | −19 | gb105pln | Nicotiana tabacum (TSC40-4) 60S ribosomal protein L34 mRNA, complete cds. | |
| 700344112H1 | g168496 | 89 | −65 | gb105pln | Maize (Zea mays) histone H3 gene (H3C4), complete cds. | |
| 700346816H1 | g1786697 | 13 | 2 | gb105allp | f305; This 305 aa ORF is 33 pct identical (5 gaps) to 202 residues of an approx. 288 aa | |
| | | | | | protein BAN7_HUMAN SW: P27105 | |
| 700344001H1 | g22324 | 54 | 6 | gb105pln | Z. mays mRNA for H2B histone (clone cH2B221). | |
| 700342746H1 | g968901 | 56 | −46 | gb105pln | Rice mRNA for ribosomal protein S8, complete cds. | |
| 700347319H1 | g22651 | 47 | −39 | gb105pln | H. vulgare mRNA for GRP94 homologue. | |
| 700345706H1 | g167284 | 15 | 7 | gb105eukp | glyoxysomal malate dehydrogenase precursor (EC 1.1.1.37) | |
| 700342459H1 | g2282583 | 74 | −66 | gb105pln | Zea mays elongation factor 1-alpha (EF1-A) mRNA, complete cds. | |
| 700343406H1 | g1905943 | 29 | −38 | gb105pln | Sorghum bicolor MADS box transcription factor SbMADS1 mRNA, complete cds. | |
| 700343183H1 | g19257 | 41 | −37 | gb105pln | Lycopersicon esculentum hsc-2 mRNA for heat shock protein cognate 70. | |
| 700345465H1 | g758354 | 49 | −53 | gb105pln | Z. mays mRNA for plasma membrane H+ ATPase. | |
| 700343070H1 | g2306980 | 39 | −41 | gb105pln | Oryza sativa photosystem I antenna protein (Lhca) mRNA, complete cds. | |
| 700345278H1 | g2258073 | 34 | −21 | gb105pln | Hordeum vulgare var. distichum soluble inorganic pyrophosphatase (Ipp) mRNA, | |
| | | | | | complete cds. | |
| 700347103H1 | g968901 | 35 | −43 | gb105pln | Rice mRNA for ribosomal protein S8, complete cds. | |
| 700380047H1 | g1743007 | 33 | −3 | gb105eukp | ribosomal protein L13a. | |
| 700346445H1 | g2687432 | 43 | −53 | gb105pln | Plumbago auriculata large subunit 26S ribosomal RNA gene, partial sequence. | |
| 700343906H1 | g927427 | 12 | 15 | gb105pln | L. usitatissiumum mRNA for fis1 protein. | |
| 700342866H1 | g1125808 | 60 | −5 | gb105eukp | F07D10.1 | |
| 700345122H1 | g2351069 | 12 | 11 | gb105pln | Arabidopsis thaliana genomic DNA, chromosome 5, P1 clone: MSH12. | |
| 700342655H1 | g2243116 | 29 | −5 | gb105eukp | ak-lys1; aspartate kinase; Ec 2.7.2.4 | |
| 700343339H1 | g1255116 | 17 | 5 | gb105allp | heat-responsive protein | |
| 700344882H1 | g248336 | 77 | −74 | gb105pln | polyubiquitin [maize, Genomic, 3841 nt]. | |
| 700342949H1 | g1177368 | 98 | −78 | gb105pln | Z. mays mRNA for ribosomal protein L39. | |
| 700345191H1 | g1655678 | 19 | −9 | gb105pln | P. sylvestris mRNA for 3-hydroxy-3-methylglutaryl-CoA synthase. | |
| 700379842H1 | g2465428 | 20 | −22 | gb105eukp | JRG1.2; 32 kDa protein | |
| 700346220H1 | g1184775 | 64 | −85 | gb105pln | Zea mays glyceraldehyde-3-phosphate dehydrogenase GAPC4 (gpc4) mRNA, | |
| | | | | | complete cds. | |
| 700344977H1 | g2651316 | 17 | −2 | gb105eukp | T2P4.5 | |
| 700343681H1 | g21800 | 31 | −37 | gb105pln | T. aestivum L mRNA for histone H2B. | |
| 700345652H1 | g204482 | 59 | 2 | gb105allp | glutamine/glutamic acid-rich protein | |
| 700342996H1 | g1741870 | 20 | −5 | gb105allp | SDF2 | |
| 700379995H1 | g1931628 | 55 | −31 | gb105pln | Zea mays glutamate dehydrogenase mutant (GDH1) mRNA, complete cds. | |
| 700342211H1 | g1052973 | 19 | −12 | gb105eukp | fructokinae; EC 2.7.1.4 | |
| 700343419H1 | g22322 | 70 | −54 | gb105pln | Z. mays mRNA for H2B histone (clone cH2B214). | |
| 700343101H1 | g2264367 | 24 | −7 | gb105pln | Arabidopsis thaliana BAC F6P23 from chromosome IV, top arm, complete sequence. | |
| 700344703H1 | g971713 | 52 | 6 | gb105allp | breast basic conserved protein 1 | |
| 700346249H1 | g1931637 | 12 | −2 | gb105eukp | T19D16.1; receptor-associated kinase isolog | |
| 700343183H1 | g1181672 | 59 | −61 | gb105pln | Sorghum bicolor heat shock protein 70 cognate (hsc70) mRNA, partial cds. | |
| 700343233H1 | g1628443 | 18 | −4 | gb105eukp | ORF | |
| 700380608H1 | g1658321 | 33 | −18 | gb105pln | S. tuberosum mRNA for transketolase. | |
| 700380216H1 | g416251 | 49 | −49 | gb105pln | Rice mRNA for acetohydroxy acid reductoisomerase, partial sequence. | |
| 700342828H1 | g2565339 | 39 | −26 | gb105pln | Lupinus luteus ribosomal protein S14 (rps14) mRNA, complete cds. | |
| 700347170H1 | g170164 | 24 | −36 | gb105pln | S. guttatum aox1 mRNA encoding an alternative oxidase protein. | |
| 700344836H1 | g166923 | 28 | −6 | gb105pln | Arabidopsis thaliana ubiquitin carrier protein (UBC1) mRNA, complete cds. | |
| 700344613H1 | g453281 | 44 | −2 | gb105allp | ribosomal protein S23 | |
| 700347127H1 | g1183936 | 18 | 15 | gb105pln | P. sativum 5S rRNA gene. | |
| 700346231H1 | g461232 | 12 | 7 | gb105allp | ribosomal protein L37 | |
| 700342537H1 | g949877 | 77 | −65 | gb105pln | H. vulgare mRNA for elongation factor 1-alpha. | |
| 700379982H1 | g1262435 | 19 | 6 | gb105allp | put. 26S protease subunit | |
| 700342493H1 | g38223 | 89 | −41 | gb105allp | alpha1-globin | |
| 700380791H1 | g13844 | 20 | 4 | gb105allp | ATPase 6 | |
| 700344042H1 | g515376 | 66 | −59 | gb105pln | L. temulentum mRNA for histone H4. | |
| 700342829H1 | g633686 | 20 | −1 | gb105pln | S. tuberosum (Desiree) 8.2 kD subunit of cytochrome C reductase mRNA. | |
| 700342519H1 | g167107 | 70 | −62 | gb105pln | Hordeum vulgare vacuolar ATPase B subunit isoform mRNA, complete cds. | |
| 700347319H1 | g623612 | 11 | 14 | gb105pln | Yeast (Schizosaccharomyces pombe) heat shock protein 90 (Hsp90) gene, complete cds. | |
| 700346164H1 | g415315 | 15 | −2 | gb105eukp | ME6; NADP-dependent malic enzyme | |
| 700345305H1 | g1086831 | 15 | −4 | gb105eukp | F10E7.7 | |
| 700345064H1 | g22469 | 30 | −1 | gb105pln | Maize mRNA for cytoplasmic ribosomal protein S11. | |
| 700344056H1 | g476095 | 59 | −17 | gb105allp | B4-2 protein | |
| 700344217H1 | g387908 | 21 | 8 | gb105pln | Brassica rapa S-phase-specific (BIS289) mRNA, complete cds. | |
| 700380163H1 | g57710 | 18 | 6 | gb105allp | ribosomal phosphoprotein P1 (AA 1-114) | |
| 700343126H1 | g468055 | 24 | −28 | gb105pln | Zea mays B73 QM protein mRNA, complete cds. | |
| 700347002H1 | g829147 | 90 | −76 | gb105pln | Z. mays gene for cyclophilin. | |
| 700346726H1 | g557871 | 27 | −5 | gb105eukp | cytochrome oxidase I | |
| 700344952H1 | g21832 | 37 | −23 | gb105pln | Wheat mRNA for chloroplast phosphoglycerate kinase (EC 2.7.2.3). | |
| 700346540H1 | g404864 | 11 | 6 | gb105eukp | E(z) | |
| 700346221H1 | g2160155 | 33 | −17 | gb105pln | Sequence of BAC F21M12 from Arabidospis thaliana chromosome 1, complete | |
| | | | | | sequence. | |
| 700345973H1 | g2245082 | 26 | −14 | gb105eukp | SCARECROW homolog | |
| 700345275H1 | g1136119 | 46 | −38 | gb105pln | O. sativa mRNA for alpha-tubulin (clone OSTA-111). | |
| 700345389H1 | g16086 | 18 | −21 | gb105pln | A. porrum dnaJ mRNA for DNA J protein (partial). | |
| 700344294H1 | g2494110 | 25 | −10 | gb105pln | Sequence of BAC T1G11 from Arabidopsis thaliana chromosome 1, complete | |
| | | | | | sequence. | |
| 700346201H1 | g968995 | 48 | −57 | gb105pln | Oryza sativa clone glyceraldehyde-3-phosphate dehydrogenase (Gpc) mRNA, | |
| | | | | | complete cds. | |
| 700343937H1 | g20267 | 14 | −2 | gb105eukp | lipoxygenase L-2; EC 1.13.11.12 | |
| 700343356H1 | g1184775 | 89 | −81 | gb105pln | Zea mays glyceraldehyde-3-phosphate dehydrogenase GAPC4 (gpc4) mRNA, | |
| | | | | | complete cds. | |
| 700345752H1 | g415316 | 21 | −7 | gb105pln | Rice mRNA for acidic ribosomal protein P0, complete cds. | |
| 700345254H1 | g1800220 | 27 | −14 | gb105pln | Daucus carota topoisomerase I (Top1) mRNA, complete cds. | |
| 700345690H1 | g455419 | 13 | 3 | gb105allp | PR264/SC35 | |
| 700342566H1 | g170781 | 30 | −11 | gb105pln | Wheat ubiquitin carrier protein (E2) mRNA, complete cds. | |
| 700345973H1 | g2339980 | 14 | 6 | gb105eukp | RGA2; RGA2 protein | |
| 700380596H1 | g2583081 | 7 | 4 | gb105allp | microsomal glutathione S-transferase 3 | |
| 700342434H1 | g2130984 | 38 | −21 | gb105pln | B. napus mRNA for ribosomal protein S15a (pA813). | |
| 700379634H1 | g2511540 | 54 | −47 | gb105pln | Oryza sativa DNA-binding protein GBP16 (Rgbp16) mRNA, complete cds. | |
| 700344746H1 | g514305 | 30 | −32 | gb105pln | Triticum aestivum Tam 107 Hard Red Winter acetyl-CoA carboxylase mRNA, | |
| | | | | | complete cds. | |
| 700344108H1 | g20163 | 59 | −51 | gb105pln | O. sativa Rr15 mRNA for 5S ribosomal RNA. | |
| 700343154H1 | g475212 | 12 | 15 | gb105pln | N. tabacum eIF-4A10 mRNA. | |
| 700380255H1 | g170775 | 53 | −47 | gb105pln | Wheat translation elongation factor 1 alpha-subunit (TEF1) mRNA, complete cds. | |
| 700344040H1 | g2632156 | 30 | −21 | gb105pln | Aspergillus niger mRNA for ribosomal protein L15. | |
| 700345687H1 | g529705 | 15 | 3 | gb105eukp | cct-6; C. elegans cct-6 (similar to T complex proteins, Tcp-1-related, Cctz) | |
| 700347384H1 | g950052 | 23 | −17 | gb105pln | H. vulgare mRNA for HMG1/2-like protein. | |
| 700343238H1 | g1498052 | 67 | −21 | gb105pln | Zea mays ribosomal protein S8 mRNA, complete cds. | |
| 700345396H1 | g2760165 | 15 | 13 | gb105pln | Arabidopsis thaliana genomic DNA, chromosome 5, P1 clone: MAC9, complete | |
| | | | | | sequence. | |
| 700342657H1 | g1754489 | 32 | 3 | gb105allp | prolyl aminopeptidase | |
| 700343439H1 | g21794 | 69 | −55 | gb105pln | Wheat histone H4 gene. | |
| 700347191H1 | g285951 | 16 | 3 | gb105allp | KIAA0107 | |
| 700342329H1 | g515357 | 17 | 3 | gb105pln | S. tuberosum pPOM36I mRNA for 36kDA porinI. | |
| 700346909H1 | g1107484 | 27 | −12 | gb105pln | A. thaliana mRNA for 40S ribosomal protein S15. | |
| 700346185H1 | g1638842 | 42 | −1 | gb105eukp | cysteine proteinase inhibitor | |
| 700345133H1 | g218160 | 42 | −31 | gb105pln | Oryza sativa mRNA for elongation factor 1 beta′. | |
| 700379634H1 | g1657616 | 32 | −18 | gb105pln | Arabidopsis thaliana putative nuclear DNA-binding protein G2p (AtG2) mRNA, | |
| | | | | | complete cds. | |
| 700342460H1 | g2827513 | 15 | 10 | gb105pln | Arabidopsis thaliana DNA chromosome 4, BAC clone F8F16 (ESSAII project). | |
| 700344253H1 | g2827138 | 25 | −16 | gb105pln | Arabidopsis thaliana cellulose synthase catalytic subunit (RSW1) gene, complete cds. | |
| 700380093H1 | g1335965 | 27 | −34 | gb105pln | Zea mays acetyl CoA carboxylase mRNA, partial cds. | |
| 700343926H1 | g972781 | 16 | 5 | gb105eukp | cyclophilin | |
| 700342993H1 | g396230 | 32 | −17 | gb105eukp | putative ATP synthase subunit | |
| 700346965H1 | g2160173 | 7 | 4 | gb105eukp | F21M12.21 | |
| 700345709H1 | g15988 | 57 | −63 | gb105pln | A. fatua mRNA for tetraubiquitin. | |
| 700345629H1 | g167967 | 22 | −0 | gb105pln | Dianthus caryophyllus glutathione s-transferase mRNA, complete cds. | |
| 700343810H1 | g2827001 | 65 | −58 | gb105pln | Triticum aestivum 70 kDa heat shock protein (TaHSP70d) mRNA, complete cds. | |
| 700343424H1 | g2565339 | 51 | −42 | gb105pln | Lupinus luteus ribosomal protein S14 (rps14) mRNA, complete cds. | |
| 700345831H1 | g184858 | 54 | −29 | gb105allp | immunoglobulin heavy chain V-region | |
| 700346991H1 | g1685645 | 46 | −2 | gb105pln | Zea mays putative ribosomal protein L19 homolog mRNA, partial cds. | |
| 700345584H1 | g2773188 | 40 | −21 | gb105eukp | F49D11.1 | |
| 700344633H1 | g2724049 | 35 | −21 | gb105pln | Fritillaria agrestis clone CA1 glutaredoxin type I (glr1) mRNA, complete cds. | |
| 700346337H1 | g473604 | 78 | −62 | gb105pln | Zea mays W-22 histone H2B mRNA, complete cds. | |
| 700344944H1 | g555616 | 35 | −12 | gb105eukp | cim1; cytokinin induced message | |
| 700281955H2 | g433043 | 35 | −96 | gb105pln | Zea mays W-22 clone PREM-1E retroelement PREM-1, partial sequence. | |
| 700379974H1 | g577611 | 92 | −77 | gb105pln | Zea mays CRT1 gene for calcium-binding protein. | |
| 700346263H1 | g1931647 | 18 | 3 | gb105eukp | T19D16.13; endomembrane protein EMP70 precursor isolog | |
| 700345596H1 | g1438843 | 27 | 7 | gb105pln | Arabidopsis thaliana katC mRNA for heavy chain of kinesin-like protein, complete cds. | |
| 700345133H1 | g218340 | 37 | −37 | gb105pln | Triticum aestivum mRNA for elongation factor 1 beta′. | |
| 700344193H1 | g182251 | 15 | 5 | gb105allp | electron transport flavoprotein | |
| 700343952H1 | g22302 | 40 | −79 | gb105pln | Maize Gpc1 gene for glyceraldehyde-3-phosphate dehydrogenase (GADPH) subunit C. | |
| 700346251H1 | g173065 | 20 | 2 | gb105pln | Yeast (Saccharomyces cerevisiae) mitochondrial ef-tu (tufm) gene. | |
| 700345661H1 | g63752 | 17 | 1 | gb105allp | PR 264 | |
| 700344053H1 | g1370167 | 56 | −37 | gb105pln | L. japonicus mRNA for small GTP-binding protein, RAB1D. | |
| 700342287H1 | g578440 | 7 | −4 | gb105eukp | ATP6; H(+)-transporting ATP synthase; EC 3.6.1.34 | |
| 700345677H1 | g2624199 | 58 | −43 | gb105pln | M. acuminata mRNA; clone pBAN UU93. | |
| 700343937H1 | g2182267 | 18 | −1 | gb105eukp | LoxB; lipoxygense; EC 1.13.11.12 | |
| 700343244H1 | g1917018 | 100 | −76 | gb105pln | Zea mays ribosomal protein S6 RPS6-1 (rps6-1) mRNA, complete cds. | |
| 700380162H1 | g949877 | 44 | −34 | gb105pln | H. vulgare mRNA for elongation factor 1-alpha. | |
| 700342366H1 | g22747 | 20 | −0 | gb105pln | L. longiflorum mRNA for actin depolymerizing factor. | |
| 700281918H2 | g510190 | 16 | 3 | gb105eukp | unknown; chloroplast outer envelope protein 34 | |
| 700343566H1 | g1419369 | 30 | −27 | gb105pln | Z. mays ZmABP3 mRNA for actin depolymerizing factor. | |
| 700346096H1 | g170746 | 46 | 6 | gb105pln | Wheat histone H4 TH091 gene, complete cds. | |
| 700342732H1 | g2293565 | 80 | −77 | gb105pln | Oryza sativa ADP-ribosylation factor 1 (Os-ARF1) mRNA, complete cds. | |
| 700342447H1 | g2677829 | 38 | −18 | gb105pln | Prunus armeniaca ribosomal protein L12 mRNA, complete cds. | |
| 700380443H1 | g1200160 | 16 | −10 | gb105pln | T. gesneriana mRNA for tonoplast intrinsic protein. | |
| 700346459H1 | g170746 | 54 | −53 | gb105pln | Wheat histone H4 TH091 gene, complete cds. | |
| 700344524H1 | g168472 | 42 | −15 | gb105pln | Maize ferredoxin III (Fd) isoprotein mRNA, pFD3. | |
| 700346122H1 | g1777929 | 24 | −25 | gb105pln | Saccharum officinarum nucleoside diphosphate kinase (SoNDPK1) mRNA, | |
| | | | | | complete cds. | |
| 700342493H1 | g386765 | 89 | −41 | gb105allp | hba1 alpha globin | |
| 700344101H1 | g189422 | 9 | 7 | gb105allp | proliferating cell nuclear protein P120 | |
| 700347353H1 | g393400 | 82 | −78 | gb105pln | Z. mays mRNA for alpha-tubulin. | |
| 700344029H1 | g2522364 | 30 | 1 | gb105eukp | atMSH2; MutS homolog 2 | |
| 700343282H1 | g2244788 | 17 | −20 | gb105pln | Arabidopsis thaliana DNA chromosome 4, ESSA I contig fragment No. 1. | |
| 700343303H1 | g22322 | 27 | −45 | gb105pln | Z. mays mRNA for H2B histone (clone cH2B214). | |
| 700347255H1 | g21438 | 40 | −38 | gb105pln | S. tuberosum cycl gene encoding cytochrome c1. | |
| 700345418H1 | g1325967 | 28 | −11 | gb105pln | T. aestivum histone H2A gene (clone TH274). | |
| 700379678H1 | g1129084 | 33 | −27 | gb105pln | Wheat mRNA for protein H2A, complete cds, clone wcH2A-3. | |
| 700346867H1 | g168608 | 95 | −74 | gb105pln | Maize 17S ribosomal RNA gene and flanks. | |
| 700344149H1 | g2435604 | 20 | −1 | gb105eukp | F08F1.7 | |
| 700344073H1 | g2088662 | 12 | 2 | gb105eukp | T28M21.22 | |
| 700346985H1 | g168500 | 51 | −50 | gb105pln | Maize (Zea mays) histone H4 gene (H4C14), complete cds. | |
| 700346803H1 | g1458245 | 10 | −0 | gb105eukp | F54D11.1 | |
| 700380511H1 | g533689 | 30 | −21 | gb105pln | Brassica napus Naehan RNA polymerase II subunit RPB10 homolog (BRP10) mRNA, | |
| | | | | | complete cds. | |
| 700345690H1 | g63752 | 13 | 3 | gb105allp | PR 264 | |
| 700345632H1 | g2570506 | 45 | −31 | gb105pln | Oryza sativa ribosomal protein mRNA, complete cds. | |
| 700342267H1 | g293886 | 96 | −84 | gb105pln | Zea mays glyceraldehyde-3-phosphate dehydrogenase mRNA, 3′ end, (clone GAPC3). | |
| 700346347H1 | g1255410 | 19 | −15 | gb105eukp | T19A5.2 | |
| 700344757H1 | g306812 | 34 | −17 | gb105allp | glutathione transferase M1 | |
| 700342528H1 | g218182 | 41 | −24 | gb105pln | Rice mRNA for oryzain beta (EC 3.4.22). | |
| 700379970H1 | g168500 | 63 | −32 | gb105pln | Maize (Zea mays) histone H4 gene (H4C14), complete cds. | |
| 700379813H1 | g2244991 | 20 | −19 | gb105pln | Arabidopsis thaliana DNA chromosome 4, ESSA I contig fragment No. 6. | |
| 700344966H1 | g391878 | 35 | −41 | gb105pln | Rice mRNA for adenylate kinase. | |
| 700344790H1 | g1458245 | 17 | −2 | gb105eukp | F54D11.1 | |
| 700343229H1 | g2274990 | 54 | −33 | gb105pln | Hordeum vulgare mRNA for expressed sequence tag. | |
| 700343305H1 | g288058 | 75 | −64 | gb105pln | Z. mays S13 mRNA for cytoplasmic ribosomal protein S13. | |
| 700344865H1 | g602605 | 27 | −29 | gb105pln | Zea mays tandem genes for alpha1-tubulin and alpha2-tubulin. | |
| 700346914H1 | g433608 | 12 | 1 | gb105pln | R. communis mRNA for enolase. | |
| 700344612H1 | g2511530 | 80 | −70 | gb105pln | Eleusine indica alpha tubulin 1 (TUA1) mRNA, complete cds. | |
| 700344330H1 | g4554 | 15 | −1 | gb105eukp | STE11 (AA 1-717) | |
| 700343205H1 | g2062696 | 28 | −18 | gb105allp | Ro/SSA ribonucleoprotein homolog | |
| 700345227H1 | g887571 | 13 | 1 | gb105allp | glycogen (starch) synthase | |
| 700345357H1 | g303856 | 49 | −33 | gb105pln | Rice mRNA for ubiquitin protein fused to a ribosomal protein, complete cds. | |
| 700346740H1 | g386848 | 18 | 4 | gb105allp | keratin | |
| 700345757H1 | g1653931 | 18 | 7 | gb105allp | argininosuccinate synthetase | |
| 700344982H1 | g1302195 | 8 | 1 | gb105allp | ORF YNL197c | |
| 700343982H1 | g2570860 | 33 | 0 | gb105allp | calcium-activated potassium channel beta subunit | |
| 700344738H1 | g2213534 | 20 | −5 | gb105eukp | DNA-binding PD1-like protein | |
| 700346727H1 | g170746 | 74 | −67 | gb105pln | Wheat histone H4 TH091 gene, complete cds. | |
| 700344146H1 | g520494 | 6 | 4 | gb105eukp | cytoskeletal actin | |
| 700343414H1 | g456671 | 24 | −3 | gb105pln | T. aestivum VDAC 1 mRNA. | |
| 700342313H1 | g3075 | 20 | −0 | gb105pln | N. crassa mRNA for a ribosomal protein. | |
| 700344450H1 | g2668738 | 50 | 8 | gb105eukp | TIF5A; translation initiation factor 5A | |
| 700345531H1 | g1871360 | 10 | 2 | gb105eukp | glutamyl-prolyl-tRNA synthetase | |
| 700345452H1 | g633139 | 36 | −22 | gb105pln | A. thaliana mRNA for phosphoribosyl diphosphate synthetase. | |
| 700344636H1 | g22237 | 96 | −4 | gb105pln | Maize mRNA for cytosolic GAPDH (GapC) glyceraldehyde-3-phosphate | |
| | | | | | dehydrogenase. | |
| 700345973H1 | g2569938 | 14 | 6 | gb105eukp | GAI | |
| 700345689H1 | g2702261 | 24 | −3 | gb105pln | Arabidopsis thaliana chromosome II BAC T21L14 genomic sequence, complete | |
| | | | | | sequence. | |
| 700380159H1 | g22484 | 45 | −45 | gb105pln | Z. mays RNA for superoxide dismutase Sod4A. | |
| 700347254H1 | g168500 | 50 | −51 | gb105pln | Maize (Zea mays) histone H4 gene (H4C14), complete cds. | |
| 700347169H1 | g17795 | 22 | −7 | gb105eukp | Bplo | |
| 700342944H1 | g1360175 | 15 | −2 | gb105eukp | ORF YLL010c | |
| 700342268H1 | g2656016 | 10 | 4 | gb105allp | cell-cycle regulatory protein | |
| 700346165H1 | g14199 | 62 | −2 | gb105pln | Petunia sp. mitochondrial rps19 gene for ribosomal protein S19. | |
| 700345355H1 | g1532045 | 19 | −12 | gb105pln | O. sativa mRNA for expansin. | |
| 700345002H1 | g557681 | 50 | −31 | gb105pln | Z. mays mRNA for ribosomal protein S28. | |
| 700343851H1 | g496267 | 22 | −5 | gb105pln | Nicotiana tabacum GTP-binding protein (Ran-A1) mRNA, complete cds. | |
| 700343453H1 | g510910 | 58 | −35 | gb105pln | L. temulentum mRNA for histone H3. | |
| 700343104H1 | g187608 | 20 | −7 | gb105allp | cell surface glycoprotein | |
| 700347390H1 | g1947005 | 19 | −8 | gb105eukp | T21G5.5 | |
| 700344393H1 | g21399 | 37 | −19 | gb105pln | Solanum tuberosum processed actin pseudogene. | |
| 700379968H1 | g1848280 | 44 | −70 | gb105pln | Sorghum bicolor membrane intrinsic protein (Mip1) mRNA, partial cds. | |
| 700342791H1 | g20283 | 39 | −6 | gb105pln | Rice (O. sativa) gene for proliferating cell nuclear antigen (PCNA). | |
| 700345227H1 | g21471 | 12 | 1 | gb105allp | glycogen (starch) synthase | |
| 700342714H1 | g2384759 | 47 | −42 | gb105pln | Oryza sativa GDP dissociation inhibitor protein OsGDI2 (OsGDI2) mRNA, | |
| | | | | | complete cds. | |
| 700343550H1 | g172424 | 13 | 5 | gb105eukp | 5S rRNA binding protein; ribosomal protein L1 | |
| 700345902H1 | g1136784 | 20 | −17 | gb105pln | Fission Yeast mRNA for elongation factor 1 alpha-B, complete cds. | |
| 700344831H1 | g1657425 | 31 | −3 | gb105eukp | tifA; tifA | |
| 700346649H1 | g2264309 | 19 | 6 | gb105pln | Arabidopsis thaliana genomic DNA, chromosome 5, P1 clone: MJJ3, complete sequence. | |
| 700344859H1 | g1575534 | 13 | 5 | gb105allp | Mad2 | |
| 700380508H1 | g1890351 | 18 | −7 | gb105pln | A. thaliana mRNA for transcription factor L2. | |
| 700346231H1 | g1215986 | 19 | 1 | gb105allp | ribosomal protein L37 | |
| 700346064H1 | g2695701 | 10 | 5 | gb105allp | mammary tumor-associated protein INT6 | |
| 700379944H1 | g218135 | 35 | −53 | gb105pln | Rice mRNA for heat shock protein 90 KD (AK108 gene), partial sequence. | |
| 700379791H1 | g168650 | 42 | −34 | gb105pln | Zea mays ubiquitin fusion protein (UBF9) gene, complete cds. | |
| 700379705H1 | g2626751 | 16 | −19 | gb105pln | Triticum aestivum retrotransposon Tar1, partial sequence. | |
| 700344234H1 | g606969 | 29 | −14 | gb105pln | Arabidopsis thaliana cytoplasmic ribosomal protein L18 mRNA, complete cds. | |
| 700281971H1 | g436032 | 44 | 3 | gb105eukp | 60S ribosomal protein L34 | |
| 700344139H1 | g22144 | 74 | −78 | gb105pln | Maize anaerobically regulated gene for fructose bisphosphate aldolase (EC 4.1.2.13). | |
| 700342458H1 | g1142621 | 13 | 7 | gb105allp | phaeolin G-box binding protein PG2 | |
| 700345257H1 | g2737908 | 13 | 6 | gb105eukp | crps-7; ribosomal protein | |
| 700380047H1 | g557790 | 35 | −5 | gb105eukp | orf, len: 199, CAI: 0.62, predicted to be spliced, similar to A44367 A44367 P23 = | |
| | | | | | TUMOR-SPECIFIC TRANSPLANTATION ANTIGEN and ribosomal proteins | |
| 700281828H2 | g899607 | 59 | −45 | gb105pln | Zea mays polyubiquitin (MubC5) mRNA, complete cds. | |
| 700344907H1 | g22324 | 14 | −16 | gb105pln | Z. mays mRNA for H2B histone (clone cH2B221). | |
| 700379926H1 | g21598 | 33 | −35 | gb105pln | S. tuberosum mRNA for UDP-glucose pyrophosphorylase. | |
| 700347372H1 | g2827699 | 13 | 1 | gb105eukp | F6H11.10; predicted protein | |
| 700347368H1 | g1418578 | 7 | 6 | gb105allp | R11D1.8 | |
| 700345467H1 | g2264306 | 19 | −1 | gb105pln | Arabidopsis thaliana genomic DNA, chromosome 5, P1 clone: MBK5, complete | |
| | | | | | sequence. | |
| 700343131H1 | g2660676 | 24 | −21 | gb105eukp | T19K24.16; Dreg-2 like protein | |
| 700380256H1 | g65317 | 16 | 2 | gb105allp | snRNP C | |
| 700346904H1 | g433608 | 56 | −47 | gb105pln | R. communis mRNA for enolase. | |
| 700342280H1 | g2852444 | 21 | −19 | gb105pln | Salix bakko mRNA for SUI1 homolog, complete cds. | |
| 700379889H1 | g886470 | 62 | −22 | gb105pln | C. roseus MetE mRNA for methionine synthase. | |
| 700342846H1 | g2072023 | 19 | 4 | gb105eukp | symbiosis-related protein | |
| 700344696H1 | g2522261 | 28 | 2 | gb105allp | CPE-receptor | |
| 700342526H1 | g2773225 | 9 | 3 | gb105eukp | W03G9.4 | |
| 700343489H1 | g2266661 | 66 | −39 | gb105pln | Hordeum vulgare mRNA for 14-3-3 protein (Hv1433c). | |
| 700380110H1 | g8040 | 22 | −8 | gb105eukp | histone H2A variant (AA 1-141) | |
| 700344147H1 | g1304268 | 21 | −7 | gb105pln | Yeast cps8 gene for actin, complete cds. | |
| 700343796H1 | g2315645 | 16 | 2 | gb105allp | strong similarity to the insect-type alcohol dehydrogenase family | |
| 700379665H1 | g1890353 | 29 | −13 | gb105pln | B. napus mRNA for ascorbate peroxidase. | |
| 700281910H1 | g313148 | 11 | 7 | gb105eukp | MSK-3; protein kinase | |
| 700380658H1 | g1854445 | 32 | 7 | gb105eukp | CPRD14 protein | |
| 700346887H1 | g1143863 | 22 | 13 | gb105pln | Oryza sativa beta-glucosidase mRNA, nuclear gene encoding chloroplast protein, | |
| | | | | | complete cds. | |
| 700345758H1 | g487317 | 42 | 11 | gb105pln | Rice mRNA EN56, partial sequence. | |
| 700344914H1 | g166859 | 41 | −33 | gb105pln | Arabidopsis thaliana ribosomal protein gene, complete cds. | |
| 700344350H1 | g507770 | 46 | −6 | gb105pln | Zea mays D3L H(+)-transporting ATPase (Mha1) gene, complete cds. | |
| 700343864H1 | g927571 | 97 | −57 | gb105pln | Z. mays mRNA for calreticulin precursor. | |
| 700343248H1 | g2623247 | 35 | −10 | gb105pln | Zea mays SU1 isoamylase (sugaryl) gene, complete cds. | |
| 700346731H1 | g556685 | 70 | −69 | gb105pln | Z. mays mRNA for ADP-ribosylation factor. | |
| 700346421H1 | g21800 | 26 | −20 | gb105pln | T. aestivum L mRNA for histone H2B. | |
| 700343051H1 | g1323417 | 24 | −12 | gb105eukp | ORF YGR231c | |
| 700344619H1 | g166604 | 13 | 7 | gb105eukp | anthranilate synthase alpha subunit | |
| 700343821H1 | g18903 | 33 | −17 | gb105pln | Barley mRNA for aspartic proteinase. | |
| 700345594H1 | g1184773 | 61 | −72 | gb105pln | Zea mays glyceraldehyde-3-phosphate dehydrogenase GAPC3 (gpc3) mRNA, | |
| | | | | | complete cds. | |
| 700344372H1 | g2804455 | 12 | 7 | gb105allp | similar to a human orf (GB:D13642) and human UV-damaged DNA binding factor | |
| | | | | | (GB:U32986) in separate non-overlapping regions | |
| 700342330H1 | g218155 | 13 | 2 | gb105allp | chloroplastic aldolase | |
| 700343221H1 | g1132506 | 30 | −17 | gb105eukp | F28C6.7 | |
| 700380795H1 | g4433 | 21 | −12 | gb105eukp | SEC11 | |
| 700345490H1 | g1054845 | 17 | −11 | gb105eukp | END13 | |
| 700345737H1 | g1001957 | 6 | 8 | gb105allp | ZNF127 | |
| 700342791H1 | g408231 | 29 | 2 | gb105pln | PCNA = proliferating cell nuclear antigen [Brassica napus = oilseed rape, cv. Westar, | |
| | | | | | apical meristem, mRNA, 1004 nt]. | |
| 700344804H1 | g550543 | 40 | −27 | gb105pln | A. thaliana mRNA for ribosomal protein L16. | |
| 700344711H1 | g602605 | 33 | −64 | gb105pln | Zea mays tandem genes for alpha1-tubulin and alpha2-tubulin. | |
| 700281891H2 | g1065890 | 9 | −0 | gb105eukp | SPAC30D11.03; unknown | |
| 700346437H1 | g2668739 | 35 | −19 | gb105pln | Zea mays translation initiation factor GOS2 (TIF) mRNA, complete cds. | |
| 700345941H1 | g473602 | 48 | −17 | gb105pln | Zea mays W-22 histone H2A mRNA, complete cds. | |
| 700346108H1 | g1841398 | 27 | −8 | gb105pln | A. thaliana mRNA for AtTFIIB2 protein. | |
| 700342760H1 | g1053066 | 35 | −17 | gb105pln | Solanum lycopersicum small GTP-binding protein (LeRab1C) mRNA, complete cds. | |
| 700343923H1 | g1139585 | 18 | −6 | gb105eukp | CCA1; addition of 3′ CMP and AMP to transfer RNAs; tRNA nucleotidyltransferase; | |
| | | | | | EC 2.7.7.25 | |
| 700343457H1 | g434902 | 30 | −13 | gb105eukp | Bap; beta-adaptin Drosophila 1 | |
| 700346780H1 | g450879 | 44 | −50 | gb105pln | A. thaliana hsc70-1 gene. | |
| 700281955H2 | g433039 | 27 | −84 | gb105pln | Zea mays W-22 clone PREM-1 retroelement PREM-1, partial sequence. | |
| 700346002H1 | g498738 | 50 | −52 | gb105pln | H. vulgare (pMaW20) pseudo mRNA for beta-ketoacyl-ACP synthase (partial). | |
| 700345552H1 | g1439533 | 20 | −22 | gb105pln | Ipomoea batatas starch phosphorylase gene, complete cds. | |
| 700344238H1 | g22204 | 40 | −18 | gb105pln | Maize Bz-McC gene for UDP glucose flavonoid glycosyl-transferase. | |
| 700343792H1 | g2827618 | 24 | −1 | gb105pln | Arabidopsis thaliana DNA chromosome 4, BAC clone F10N7 (ESSAII project). | |
| 700379938H1 | g1835728 | 36 | −25 | gb105pln | Oryza sativa ribosomal protein mRNA, complete cds. | |
| 700380290H1 | g2282583 | 59 | −48 | gb105pln | Zea mays elongation factor 1-alpha (EF1-A) mRNA, complete cds. | |
| 700380074H1 | g2827001 | 38 | −34 | gb105pln | Triticum aestivum 70 kDa heat shock protein (TaHSP70d) mRNA, complete cds. | |
| 700344894H1 | g1183998 | 18 | −0 | gb105eukp | SPAC13F4.02c; unknown | |
| 700342663H1 | g2384956 | 13 | −2 | gb105eukp | T15B7.2 | |
| 700342465H1 | g1149509 | 26 | −4 | gb105allp | 37 kD Laminin receptor precursor/p40 ribosomal associated protein | |
| 700346731H1 | g1132482 | 71 | −69 | gb105pln | Rice mRNA for ADP-ribosylation factor, complete cds. | |
| 700346294H1 | g561663 | 23 | −47 | gb105pln | Rice mRNA, partial homologous to ribosomal protein coding sequence. | |
| 700342464H1 | g2191175 | 28 | −5 | gb105eukp | A_IG002P16.24 | |
| 700344445H1 | g1545982 | 15 | −5 | gb105allp | PWP2H protein | |
| 700343506H1 | g1498052 | 29 | −60 | gb105pln | Zea mays ribosomal protein S8 mRNA, complete cds. | |
| 700345692H1 | g2827001 | 31 | −41 | gb105pln | Triticum aestivum 70 kDa heat shock protein (TaHSP70d) mRNA, complete cds. | |
| 700343864H1 | g1181332 | 97 | −57 | gb05pln | Z mays CRH mRNA. | |
| 700345441h1 | g506533 | 43 | −31 | gb105pln | N. tabacum PKTL7 mRNA for protein kinase. | |
| 700379902H1 | g2651307 | 24 | 2 | gb105eukp | T2P4.16; putative uroporphyinogen decarboxylase | |
| 700345992H1 | g2414643 | 25 | −5 | gb105pln | Yeast (Saccaromyces pombe) chromosome I cosmid c3H5. | |
| 700344792H1 | g1486425 | 13 | −1 | gb105allp | BioA homologue | |
| 700342479H1 | g1339835 | 17 | −4 | gb105allp | kidney androgen-related protein | |
| 700343945H1 | g1519248 | 42 | −25 | gb105pln | Oryza sativa GF14-b protein mRNA, complete cds. | |
| 700344931H1 | g1667591 | 55 | −43 | gb105pln | Oryza sativa histone 3 mRNA, complete cds. | |
| 700343277H1 | g643596 | 86 | −82 | gb105pln | Corn mRNA for cysteine proteinase, clone CCP, complete cds. | |
| 700346868H1 | g1495767 | 21 | −1 | gb105pln | P. sativum mRNA for 110 kD chloroplast inner envelope protein IEP110. | |
| 700344390H1 | g2695860 | 23 | −1 | gb105pln | Pisum sativum mRNA for 3-deoxy-D-manno-2-octulosonate-8-phosphate synthase, | |
| | | | | | clone pPS40. | |
| 700342772H1 | g20163 | 52 | −44 | gb105pln | O. sativa Rr15 mRNA for 5S ribosomal RNA. | |
| 700342648H1 | g170046 | 7 | 17 | gb105pln | Glycine max protein kinase (PK6) mRNA, complete cds. | |
| 700281977H2 | g167109 | 42 | −36 | gb105pln | Hordeum vulgare vacuolar ATPase B subunit mRNA, complete cds. | |
| 700344851H1 | g531057 | 14 | 3 | gb105pln | Wheat mRNA for protein H2B-8, complete cds. | |
| 700379649H1 | g899609 | 65 | −54 | gb105pln | Zea mays acidic ribosomal protein P2 (RPA-2A1) mRNA, complete cds. | |
| 700345009H1 | g1905933 | 41 | −24 | gb105pln | Sorghum bicolor putative MADS box protein (SbMADS2) mRNA, partial cds. | |
| 700344064H1 | g556685 | 62 | −62 | gb105pln | Z. mays mRNA for ADP-ribosylation factor. | |
| 700343054H1 | g1276967 | 15 | −2 | gb105eukp | putative ribosomal protein | |
| 700345183H1 | g487304 | 16 | −10 | gb105pln | Rice mRNA EN320, partial sequence. | |
| 700344369H1 | g551251 | 9 | 1 | gb105allp | ribosomal protein S7 | |
| 700379675H1 | g1419464 | 12 | −6 | gb105eukp | ZC434.2 | |
| 700343502H1 | g623577 | 20 | −17 | gb105pln | Nicotiana tabacum SR1 Nt-rab11b mRNA, complete cds. | |
| 700342295H1 | g1160445 | 5 | 8 | gb105allp | ribosomal protein S7 | |
| 700343391H1 | g22312 | 54 | −63 | gb105pln | Maize ABA-inducible gene for glycine-rich protein (ABA = abscisic acid). | |
| 700343840H1 | g393400 | 83 | −71 | gb105pln | Z. mays mRNA for alpha-tubulin. | |
| 700343925H1 | g1773257 | 18 | 4 | gb105allp | aspartyl-tRNA synthetase | |
| 700343809H1 | g1498387 | 41 | −52 | gb105pln | Zea mays actin (Maz83) gene, partial cds. | |
| 700345913H1 | g1086831 | 14 | −2 | gb105eukp | F10E7.7 | |
| 700345543H1 | g2832401 | 30 | −17 | gb105pln | Arabidopsis thaliana mRNA for dihydroxypolyprenylbenzoate methyltransferase. | |
| 700344254H1 | g467467 | 9 | −8 | gb105allp | unknown | |
| 700344085H1 | g2668749 | 64 | −65 | gb105pln | Zea mays ribosomal protein L30 (rp130) mRNA, complete cds. | |
| 700379858H1 | g881485 | 9 | 6 | gb105allp | extendin | |
| 700379614H1 | g602605 | 36 | −63 | gb105pln | Zea mays tandem genes for alpha1-tubulin and alpha2-tubulin. | |
| 700344527H1 | g829137 | 15 | 0 | gb105eukp | ORF OR26.17; STI1 heat shock protein; STI1 | |
| 700343526H1 | g2062169 | 16 | 1 | gb105eukp | T02O04.17; ABC transporter (PDR5-like) isolog | |
| 700342534H1 | g473602 | 91 | −71 | gb105pln | Zea mays W-22 histone H2A mRNA, complete cds. | |
| 700347376H1 | g817824 | 16 | 4 | gb105eukp | SEC26; Sec26p | |
| 700347290H1 | g1902902 | 65 | −54 | gb105pln | Oryza sativa DNA for phospholipase D, complete cds. | |
| 700342619H1 | g170746 | 32 | −42 | gb105pln | Wheat histone H4 Th091 gene, complete cds. | |
| 700380716H1 | g1817577 | 40 | −22 | gb105allp | alpha-globin 1 | |
| 700342245H1 | g471319 | 17 | 5 | gb105eukp | HvB12D | |
| 700347305H1 | g887588 | 7 | 6 | gb105eukp | unknown | |
| 700343691H1 | g2108346 | 25 | −7 | gb105pln | Brassica campestris small GTP-binding protein Bsar1b (bsar1b) mRNA, complete cds. | |
| 700342712H1 | g456355 | 42 | −53 | gb105pln | N. tabacum NtK-1 mRNA. | |
| 700343461H1 | g409072 | 16 | 6 | gb105allp | HBp15/L22 | |
| 700344420H1 | g960356 | 10 | 0 | gb105pln | Barley mRNA for S-adenosylmethionine synthetase, complete cds. | |
| 700346547H1 | g2351061 | 16 | −3 | gb105pln | Arabidopsis thaliana genomic DNA, chromosome 5, P1 clone: MAF19. | |
| 700343018H1 | g2814786 | 18 | −8 | gb105eukp | C13B4.2 | |
| 700346560H1 | g2832707 | 14 | −3 | gb105eukp | T9A21.180; translation initiation factor eIF-2 gamma chain-like protein | |
| 700346373H1 | g563926 | 19 | −18 | gb105pln | Zea mays xyloglucan endo-transglycosylase homolog mRNA, complete cds. | |
| 700345652H1 | g459931 | 59 | 2 | gb105allp | contiguous repeat polypeptide | |
| 700344093H1 | g217973 | 34 | −55 | gb105pln | Zea mays gene for triosephosphate isomerase, complete cds. | |
| 700347041H1 | g1945611 | 7 | 8 | gb105allp | 26S proteasome subunit p55 | |
| 700344856H1 | g2245038 | 16 | −6 | gb105eukp | hypothetical protein | |
| 700346225H1 | g1016205 | 28 | −16 | gb105eukp | clpP1; subunit of ClpP protease | |
| 700343947H1 | g1302630 | 9 | 3 | gb105allp | DUB-1 | |
| 700342557H1 | g303834 | 32 | −45 | gb105pln | Rice mRNA for 21 kd polypeptide, complete cds. | |
| 700346533H1 | g473976 | 28 | −29 | gb105pln | Rice mRNA, partial homologous to elongation factor 1-alpha gene. | |
| 700346391H1 | g1743276 | 38 | −15 | gb105pln | H. vulgare mRNA for beta tubulin. | |
| 700379653H1 | g19400 | 20 | 3 | gb105pln | Lemna gibba negatively light-regulated mRNA (Lg106). | |
| 700345109H1 | g1469041 | 5 | 7 | gb105eukp | F23B12.5 | |
| 700343846H1 | g1498052 | 77 | −66 | gb105pln | Zea mays ribosomal protein S8 mRNA, complete cds. | |
| 700281833H2 | g2440041 | 17 | −1 | gb105pln | Craterostigma plantagineum mRNA for major intrinsic protein PIPa2. | |
| 700342809H1 | g1654143 | 45 | −34 | gb105pln | Brassica campestris small GTP-binding protein rab (BRAB-1) mRNA, complete cds. | |
| 700342885H1 | g486279 | 12 | 4 | gb105allp | ORF YKL160w | |
| 700380108H1 | g2673867 | 23 | −4 | gb105pln | Antirrhinum majus mRNA for fimbriata-associated protein 1, partial. | |
| 700346284H1 | g218082 | 24 | −19 | gb105pln | Rice mRNA for initiation factor eIF-4D (225 gene), partial sequence. | |
| 700380133H1 | g531057 | 16 | −7 | gb105pln | Wheat mRNA for protein H2B-8, complete cds. | |
| 700346679H1 | g2653284 | 18 | 12 | gb105pln | Oryza sativa mRNA for enoyl-ACP reductase. | |
| 700344974H1 | g473604 | 39 | −22 | gb105pln | Zea mays W-22 histone H2B mRNA, complete cds. | |
| 700342751H1 | g559921 | 52 | 5 | gb105eukp | axi 1 | |
| 700346622H1 | g170775 | 61 | −48 | gb105pln | Wheat translation elongation factor 1 alpha-subunit (TEF1) mRNA, complete cds. | |
| 700347019H1 | g886470 | 52 | −44 | gb105pln | C. roseus MetE mRNA for methionine synthase. | |
| 700342262H1 | g1065942 | 12 | 2 | gb105eukp | F42C5.8 | |
| 700343126H1 | g575354 | 10 | 15 | gb105pln | O. sativa SC34 mRNA for tumor suppressor. | |
| 700379868H1 | g1002532 | 46 | 9 | gb105pln | Arabidopsis thaliana actin-11 (ACT11) gene, complete cds. | |
| 700346762H1 | g1814400 | 44 | −36 | gb105pln | Mesembryanthemum crystallinum phosphoglucomutase (PGM) mRNA, complete cds. | |
| 700379947H1 | g2191186 | 8 | 6 | gb105allp | contains similarity to tropomyosin and kinesin | |
| 700347111H1 | g312345 | 26 | 1 | gb105allp | rat ribosomal protein L36 | |
| 700346965H1 | g2335106 | 9 | 4 | gb105allp | salt inducible protein isolog | |
| 700346381H1 | g2213606 | 24 | −1 | gb105pln | Genomic sequence for Arabidopsis thaliana BAC F21J9, complete sequence. | |
| 700346780H1 | g313759 | 50 | −57 | gb105pln | Z. mays hsp 70-1 gene for heat shock protein 70. | |
| 700346584H1 | g2342687 | 19 | 0 | gb105eukp | F7G19.20 | |
| 700343054H1 | g1669623 | 10 | 4 | gb105eukp | ribosomal protein L39 | |
| 700343168H1 | g171785 | 10 | −0 | gb105eukp | alpha-ketoglutarate dehydrogenase | |
| 700344925H1 | g452473 | 91 | −75 | gb105pln | Zea mays Black Mexican Sweet alpha-tubulin mRNA, complete cds. | |
| 700342992H1 | g1183936 | 16 | 2 | gb105pln | P. sativum 5S rRNA gene. | |
| 700343151H1 | g312257 | 25 | −10 | gb105pln | Yeast (Saccharomyces cerevisiae) PUP2 gene. | |
| 700346705H1 | g577824 | 36 | −25 | gb105pln | Z. mays gene for H2B histone (gH2B3). | |
| 700345783H1 | g2388931 | 14 | 6 | gb105eukp | SPAC23H4.12; hypothetical protein | |
| 700343718H1 | g1147799 | 22 | −3 | gb105pln | Yeast (Saccaromyces cerevisiae) Sug2p (SUG2) gene, complete cds. | |
| 700343915H1 | g1632830 | 23 | 7 | gb105pln | R. communis mRNA (unknown). | |
| 700342745H1 | g2465923 | 24 | −12 | gb105eukp | RKF1; receptor-like serine/threonine kinase | |
| 700379960H1 | g21800 | 37 | −40 | gb105pln | T. aestivum L mRNA for histone H2B. | |
| 700344646H1 | g1839580 | 35 | −3 | gb105pln | putative glycine-rich protein {clone CHEM 11} [Zea mays = maize, cv. INRA 258, | |
| | | | | | mercuric chloride-treated, leaves, mRNA Partial, 149 nt]. | |
| 700342564H1 | g436782 | 22 | −4 | gb105pln | Rice mRNA for cyc07, complete cds. | |
| 700345221H1 | g434344 | 28 | −16 | gb105pln | A. thaliana (Columbia) mRNA for S18 ribosomal protein (641 bp). | |
| 700343065H1 | g899607 | 38 | −51 | gb105pln | Zea mays polyubiquitin (MubC5) mRNA, complete cds. | |
| 700343906H1 | g927428 | 16 | 4 | gb105allp | fis1 gene product | |
| 700345571H1 | g2583135 | 15 | 5 | gb105eukp | F4L23.30 | |
| 700347255H1 | g498788 | 43 | −39 | gb105pln | S. tuberosum (Desiree) cyc1 II mRNA. for cytochrome c1. | |
| 700343958H1 | g940889 | 39 | −29 | gb105eukp | SET | |
| 700281886H2 | g487311 | 51 | 7 | gb105pln | Rice mRNA EN448, partial sequence. | |
| 700281996H2 | g396209 | 22 | 4 | gb105pln | S. polyrrhiza mRNA for D-myo-inositol-3-phosphate synthase. | |
| 700343001H1 | g173384 | 40 | −17 | gb105eukp | pold; DNA polymerase delta | |
| 700345364H1 | g1129083 | 41 | −25 | gb105pln | Wheat mRNA for protein H2A, complete cds, clone wcH2A-2. | |
| 700344101H1 | g287723 | 9 | 7 | gb105allp | P120 antigen | |
| 700343579H1 | g2564046 | 14 | −0 | gb105pln | Arabidopsis thaliana genomic DNA, chromosome 5, P1 clone: MGI19, complete | |
| | | | | | sequence. | |
| 700342683H1 | g3040 | 9 | 6 | gb105eukp | NUO-12.3 | |
| 700347328H1 | g397395 | 51 | −28 | gb105pln | Z. mays MNB1b mRNA for DNA-binding protein. | |
| 700342893H1 | g2465428 | 12 | −1 | gb105eukp | JRG1.2; 32 kDa protein | |
| 700347261H1 | g248336 | 45 | −70 | gb105pln | polyubiquitin [maize, Genomic 3841 nt]. | |
| 700342434H1 | g17862 | 34 | −17 | gb105pln | B. napus mRNA for ribosomal protein S15a (pPCB8). | |
| 700345079H1 | g2618602 | 11 | 9 | gb105pln | Arabidopsis thaliana genomic DNA, chromosome 5, P1 clone: MSJ1, complete | |
| | | | | | sequence. | |
| 700343707H1 | g168505 | 16 | −18 | gb105pln | Zea mays histone H3 gene, complete cds. | |
| 700344858H1 | g596134 | 32 | −2 | gb105allp | autoantigen | |
| 700343884H1 | g2623296 | 8 | −3 | gb105eukp | T20B5.2 | |
| 700345326H1 | g832876 | 35 | −0 | gb105eukp | AFRR; ascorbate free radical reductase | |
| 700343053H1 | g2735839 | 55 | −74 | gb105pln | Sorghum bicolor ADP-glucose pyrophosphyorylase subunit SH2, transcriptional | |
| | | | | | regulator, NADPH-dependent reductase A1-a and NADPH-dependent reductase A1-b | |
| | | | | | genes, complete cds. | |
| 700343338H1 | g1468969 | 37 | −17 | gb105allp | brain acyl-CoA synthtase II | |
| 700344411H1 | g998429 | 20 | −47 | gb105pln | GRF1 = general regulatory factor [Zea mays, XL80, Genomic, 5348 nt]. | |
| 700346873H1 | g2252843 | 15 | −4 | gb105eukp | A_IG005I10.24 | |
| 700343016H1 | g217842 | 20 | −3 | gb105pln | A. thaliana mRNA for protein kinase. | |
| 700347258H1 | g556685 | 55 | −38 | gb105pln | Z. mays mRNA for ADP-ribosylation factor. | |
| 700342594H1 | g416150 | 100 | −85 | gb105pln | Zea mays beta-8 tubulin (tub8) mRNA, complete cds. | |
| 700342352H1 | g396531 | 27 | −19 | gb105eukp | ribosomal protein S19 | |
| 700343573H1 | g1245447 | 30 | −15 | gb105eukp | putative ribosomal protein S13 | |
| 700345722H1 | g2342724 | 21 | −21 | gb105eukp | T14G11.16 | |
| 700344067H1 | g2792238 | 12 | 8 | gb105eukp | r10; NBS-LRR type resistance protein | |
| 700380110H1 | g2407800 | 27 | −11 | gb105eukp | H2Av; histone H2A.F/Z | |
| 700347358H1 | g1184771 | 67 | −86 | gb105pln | Zea mays glyceraldehyde-3-phosphate dehydrogenase GAPC2 (gpc2) mRNA, | |
| | | | | | complete cds. | |
| 700343667H1 | g1546918 | 27 | −62 | gb105pln | Z. mays mRNA for translation initiation factor 5A. | |
| 700345223H1 | g1086831 | 17 | −5 | gb105eukp | F10E7.7 | |
| 700379754H1 | g303834 | 33 | 7 | gb105pln | Rice mRNA for 21 kd polypeptide, complete cds. | |
| 700345831H1 | g468235 | 56 | −30 | gb105allp | immunoglobulin heavy chain Fd region V-D-J-CH1 | |
| 700344522H1 | g218176 | 30 | −30 | gb105pln | Rice mRNA for 21 kd polypeptide. | |
| 700342756H1 | g21399 | 46 | −6 | gb105pln | Solanum tuberosum processed actin pseudogene. | |
| 700342977H1 | g21233 | 41 | −30 | gb105pln | S. oleracea AHRI mRNA for acetohydroxy acid reductoisomerase. | |
| 700346567H1 | g34031 | 18 | −1 | gb105allp | KDEL receptor | |
| 700342357H1 | g395076 | 34 | −12 | gb105pln | B. rapa ribosomal protein L37a mRNA, complete CDS. | |
| 700346820H1 | g456671 | 35 | −15 | gb105pln | T. aestivum VDAC 1 mRNA. | |
| 700346587H1 | g227594 | 20 | 2 | gb105pln | Arabidopsis thaliana chromosome II BAC T08I13 genomic sequence, complete | |
| | | | | | sequence. | |
| 700343154H1 | g1749747 | 29 | −18 | gb105pln | Fission Yeast mRNA, partial cds. | |
| 700343205H1 | g2088552 | 28 | −18 | gb105allp | No definition line found | |
| 700345357H1 | g349054 | 44 | −29 | gb105pln | Solanum tuberosum ubiquitin/ribosomal fusion protein (ubi3) gene, complete cds. | |
| 700343289H1 | g3075 | 18 | 2 | gb105pln | N. crassa mRNA for a ribosomal protein. | |
| 700346546H1 | g1322276 | 21 | −4 | gb105pln | Triticum aestivum histone H2A gene, complete cds. | |
| 700346778H1 | g2653397 | 19 | −4 | gb105eukp | EG:171D11.1 | |
| 700342701H1 | g2464874 | 10 | 4 | gb105allp | patatin | |
| 700343549H1 | g1842187 | 43 | 15 | gb105pln | B. pendula mRNA encoding mitochondrial phosphate translocator. | |
| 700347130H1 | g1217607 | 41 | 2 | gb105allp | HVB12D homologue | |
| 700343070H1 | g551046 | 46 | −50 | gb105pln | L. temulentum 1haB mRNA. | |
| 700347134H1 | g22155 | 52 | −55 | gb105pln | Z. mays mRNA for alpha-tubulin 5. | |
| 700347011H1 | g1946690 | 21 | 5 | gb105eukp | glutathione peroxidase | |
| 700346090H1 | g1053044 | 18 | −30 | gb105pln | Glycine max histone H3 gene, partial cds, clone S1. | |
| 700345480H1 | g576999 | 7 | 4 | gb105eukp | C08B11.7 | |
| 700379811H1 | g2198850 | 82 | −78 | gb105pln | Zea mays cystathionine gamma-synthase (CGS1) mRNA, complete cds. | |
| 700342533H1 | g38223 | 65 | −8 | gb105allp | alpha1-globin | |
| 700346792H1 | g303848 | 31 | −12 | gb105pln | Rice mRNA for nucleoside diphosphate kinase, complete cds. | |
| 700344114H1 | g2511690 | 20 | 5 | gb105pln | Phaseolus vulgaris Moldavian encoding cysteine proteinase precursor (clone cp41). | |
| 700281904H2 | g949979 | 93 | 11 | gb105pln | Z. mays Glossy2 locus DNA. | |
| 700345641H1 | g577824 | 25 | −2 | gb105pln | Z. mays gene for H2B histone (gH2B3). | |
| 700379675H1 | g1122378 | 16 | −4 | gb105eukp | SPAC18G6.13c; unknown | |
| 700345975H1 | g498908 | 60 | −4 | gb105eukp | RPL34; ribosomal protein L34 homolog | |
| 700342518H1 | g387398 | 74 | −35 | gb105allp | epidermal keratin type I | |
| 700342883H1 | g2642157 | 8 | 8 | gb105eukp | T5I7.5 ankyrin-like protein | |
| 700379678H1 | g1129085 | 33 | −27 | gb105pln | Wheat mRNA for protein H2A, complete cds, clone wcH2A-9. | |
| 700343907H1 | g1478271 | 16 | 4 | gb105pln | Yeast (Schizosaccharomyces pombe) cosmid 800. | |
| 700380216H1 | g21233 | 43 | −39 | gb105pln | S. oleracea AHRI mRNA for acetohydroxy acid reductoisomerase. | |
| 700379902H1 | g1009429 | 23 | 2 | gb105allp | uroporphyrinogen decarboxylase | |
| 700380547H1 | g218082 | 39 | −38 | gb105pln | Rice mRNA for initiation factor eIF-4D (225 gene), partial sequence. | |
| 700345505H1 | g473976 | 52 | −38 | gb105pln | Rice mRNA, partial homologous to elongation factor 1-alpha gene. | |
| 700344352H1 | g170455 | 49 | −49 | gb105pln | Tomato heat shock cognate protein 80 gene, 3′ end. | |
| 700281872H2 | g169804 | 58 | −3 | gb105pln | Oryza sativa DNA fragment with a miscellaneous signal and an open reading frame. | |
| 700345657H1 | g1546918 | 90 | −37 | gb105pln | Z. mays mRNA for translation initiation factor 5A. | |
| 700345121H1 | g2302294 | 18 | 2 | gb105allp | unnamed protein product | |
| 700342389H1 | g886739 | 62 | −47 | gb105pln | Z. mays histone H4 gene. | |
| 700379952H1 | g1429356 | 20 | −18 | gb105eukp | ORF D1075 | |
| 700347006H1 | g488786 | 25 | −5 | gb105pln | B. oleracea putative imbibition protein mRNA. | |
| 700345559H1 | g426441 | 41 | −25 | gb105pln | Rice mRNA for thioredoxin h, complete cds. | |
| 700346091H1 | g168379 | 26 | −20 | gb105pln | M. racemosus TEF-1 gene encoding elongation factor 1-alpha (EF-1-alpha), | |
| | | | | | complete cds. | |
| 700281955H2 | g433044 | 72 | −83 | gb105pln | Zea mays W-22 clone PREM-1D retroelement PREM-1, partial sequence. | |
| 700346387H1 | g166605 | 14 | 15 | gb105pln | Arabidopsis thaliana anthranilate synthase alpha subunit gene, complete cds. | |
| 700281927H2 | g2435510 | 15 | −0 | gb105pln | Arabidopsis thaliana BAC TM017A05. | |
| 700343493H1 | g790969 | 68 | 1 | gb105pln | Rice mRNA for aldolase C-1, complete cds. | |
| 700346464H1 | g1419036 | 15 | 4 | gb105eukp | P5CS-1; delta-1-pyrroline-5-carboxylate synthase | |
| 700346446H1 | g22677 | 13 | −2 | gb105eukp | A6; anther-specific protein | |
| 700379965H1 | g498772 | 91 | −51 | gb105pln | Z. mays (cv DH5xDH7) hsp70-4 mRNA for heat shock protein 70. | |
| 700344644H1 | g1107484 | 26 | 1 | gb105pln | A. thaliana mRNA for 40S ribosomal protein S15. | |
| 700380230H1 | g1335861 | 16 | −22 | gb105pln | Glycine max clathrin heavy chain mRNA, complete cds. | |
| 700346501H1 | g1325967 | 31 | −18 | gb105pln | T. aestivum histone H2A gene (clone TH274). | |
| 700345645H1 | g16189 | 9 | 2 | gb105eukp | ATR2; NADPH-ferrihemoprotein reductase | |
| 700379858H1 | g1890281 | 7 | 4 | gb105eukp | transformation-sensitive protein homolog | |
| 700344505H1 | g268 | 11 | −2 | gb105allp | cytochrome c oxidase subunit VIb (AA 1-86) | |
| 700342639H1 | g1066153 | 18 | −3 | gb105eukp | cysC; cysteine synthase; EC 4.2.99.8 | |
| 700343344H1 | g1498246 | 56 | −41 | gb105pln | S. oleracea mRNA for bas1 protein. | |
| 700281861H2 | g2369713 | 31 | −19 | gb105pln | Beta vulgaris cDNA for elongation factor 2. | |
| 700342245H1 | g2293567 | 21 | 10 | gb105pln | Oryza sativa HvB12D homolog mRNA, complete cds. | |
| 700345389H1 | g18259 | 20 | −26 | gb105pln | C. sativus mRNA for cs DnaJ-1. | |
| 700345065H1 | g999395 | 46 | −57 | gb105pln | HSP81-3 = heat-shock Protein [Arabidopsis thaliana = thale-cress, Genomic, 3094 nt]. | |
| 700346723H1 | g147920 | 19 | −1 | gb105allp | 3-methyladenine-DNA glycosylase I (tag) | |
| 700345565H1 | g2244747 | 56 | −45 | gb105pln | Arabidopsis thaliana DNA chromosome 4, ESSA I contig fragment No. 0. | |
| 700344835H1 | g22328 | 69 | −68 | gb105pln | Maize mRNA for a high mobility group protein. | |
| 700342358H1 | g483411 | 49 | −64 | gb105pln | Zea Mays calmodulin-binding protein mRNA, 3′ end. | |
| 700345759H1 | g2662344 | 83 | −75 | gb105pln | Oryza sativa mRNA for EF-1 alpha, complete cds. | |
| 700343903H1 | g536891 | 27 | −40 | gb105pln | Wheat mRNA for protein H2A, complete cds, clone wcH2A-4. | |
| 700342658H1 | g431176 | 10 | 6 | gb105eukp | LIM9; serine proteinase | |
| 700346313H1 | g53553 | 8 | 6 | gb105allp | 25 kDa nuclear protein | |
| 700346011H1 | g467403 | 17 | 1 | gb105allp | seryl-tRNA synthetase | |
| 700344913H1 | g393400 | 86 | −75 | gb105pln | Z. mays mRNA for alpha-tubulin. | |
| 700343905H1 | g899393 | 12 | 13 | gb105pln | Yeast (Saccharomyces cerevisiae) chromosome IV cosmid 8358. | |
| 700343543H1 | g2182286 | 18 | 9 | gb105pln | Sequence of BAC F20P5 from Arabidopsis thaliana chromosome 1, complete sequence. | |
| 700344004H1 | g2642212 | 58 | −36 | gb105pln | Zea mays nitrate-induced NOI protein gene, complete cds. | |
| 700343373H1 | g2213425 | 16 | 7 | gb105eukp | unknown; hypothetical protein | |
| 700346053H1 | g169820 | 57 | −50 | gb105pln | Oryza sativa triosephosphate isomerase (Rictpi) mRNA, complete cds. | |
| 700379927H1 | g303852 | 56 | −45 | gb105pln | Rice mRNA for ribosomal protein L3, complete cds. | |
| 700344942H1 | g773580 | 14 | 14 | gb105pln | P. sativum mRNA for heat shock protein 22. | |
| 700281918H2 | g1151244 | 18 | 0 | gb105eukp | OEP34; GTP-binding protein | |
| 700342419H1 | g432367 | 38 | −21 | gb105pln | Rice mRNA for elongation factor 1 beta, complete cds. | |
| 700347061H1 | g2384759 | 51 | −37 | gb105pln | Oryza sativa GDP dissociation inhibitor protein OsGDI2 (OsGDI2) mRNA, | |
| | | | | | complete cds. | |
| 700346623H1 | g1132482 | 41 | −19 | gb105pln | Rice mRNA for ADP-ribosylation factor, complete cds. | |
| 700346594H1 | g1272406 | 10 | 7 | gb105eukp | FKBP15-1; immunophilin | |
| 700345651H1 | g1749464 | 19 | 0 | gb105eukp | similar to Saccharomyces cerevisiae putative NDP-hexose pyrophosphopylase, | |
| | | | | | SWISS-PROT Accession Number P41940 | |
| 700342695H1 | g1167953 | 22 | −3 | gb105eukp | putative 32.6 kDa jasmonate-induced protein | |
| 700346511H1 | g2655285 | 9 | 4 | gb105allp | germin-like protein 1 | |
| 700343874H1 | g2384669 | 24 | −12 | gb105eukp | AtKT1; putative potassium transporter AtKT1p | |
| 700344953H1 | g2511571 | 22 | −2 | gb105pln | Arabidopsis thaliana mRNA for proteasome subunit prcga. | |
| 700345766H1 | g406050: | 31 | 0 | gb105pln | Yeast (Schizosaccharomyces pombe) Let1 (let1) gene, complete cds. | |
| 700344050H1 | g168452 | 39 | −24 | gb105pln | Zea mays ribosome-inactivating protein (CRIP9) mRNA, complete cds. | |
| 700342796H1 | g556685 | 45 | −31 | gb105pln | Z. mays mRNA for ADP-ribosylation factor. | |
| 700379711H1 | g170746 | 22 | −23 | gb105pln | Wheat histone H4 TH091 gene, complete cds. | |
| 700342578H1 | g169190 | 33 | −31 | gb105pln | P. hybrida 5-enolpyruvylshikimate 3-phosphate synthase mRNA, complete cds. | |
| 700342229H1 | g2266661 | 36 | −21 | gb105pln | Hordeum vulgare mRNA for 14-3-3 protein (Hv1433c). | |
| 700345545H1 | g736271 | 26 | −34 | gb105pln | O. sativa hsp70 gene for heat shock protein 70. | |
| 700344506H1 | g1255029 | 100 | 2 | gb105allp | FBP 28 | |
| 700342607H1 | g35105 | 35 | −5 | gb105allp | snRNP E protein (AA 1-92) | |
| 700346208H1 | g758352 | 65 | −77 | gb105pln | Z. mays mRNA for cysteine synthase. | |
| 700345584H1 | g575457 | 8 | 7 | gb105eukp | CDC40; Cdc40p | |
| 700345174H1 | g2632252 | 12 | 2 | gb105allp | serine/threonine kinase | |
| 700344768H1 | g1149570 | 21 | 3 | gb105pln | A. thaliana mRNA for mitochondrial elongation factor Tu. | |
| 700346108H1 | g945084 | 24 | −5 | gb105pln | Arabidopsis thaliana transcription factor TFIIB mRNA, complete cds. | |
| 700343107H1 | g1293564 | 38 | −18 | gb105pln | Arabidopsis thaliana isopentenyl diphosphate:dimethylallyl diphsophate isomerase | |
| | | | | | (IPP2) mRNA, complete cds. | |
| 700347067H1 | g1143390 | 27 | −14 | gb105eukp | HMGS; hydroxymethylglutaryl-CoA synthase; EC 4.1.3.5 | |
| 700345224H1 | g20163 | 31 | −13 | gb105pln | O. sativa Rr15 mRNA for 5S ribosomal RNA. | |
| 700345242H1 | g2564051 | 22 | −6 | gb105pln | Arabidopsis thaliana genomic DNA, chromosome 5, P1 clone: MWD9, complete | |
| | | | | | sequence. | |
| 700344691H1 | g434342 | 30 | −14 | gb105pln | A. thaliana (C24) mRNA for S18 ribosomal protein. | |
| 700343926H1 | g939726 | 16 | 5 | gb105eukp | ATCYP4; cyclophilin | |
| 700343578H1 | g2828188 | 32 | −17 | gb105pln | Arabidopsis thaliana genomic DNA, chromosome 5, TAC clone: K3I3, complete | |
| | | | | | sequence. | |
| 700380795H1 | g763367 | 21 | −12 | gb105eukp | SEC11; Sec11p | |
| 700346991H1 | g470126 | 16 | 12 | gb105pln | N. tabacum (cv. Samsun NN) L19 mRNA for ribosomal protein L19. | |
| 700342594H1 | g460988 | 76 | −65 | gb105pln | O. sativa (Arborio) Beta Tubulin mRNA, clone OSTB-34. | |
| 700281891H2 | g439260 | 17 | −14 | gb105eukp | T26G10.1 | |
| 700346211H1 | g454279 | 10 | 2 | gb105allp | DNA-binding protein | |
| 700345878H1 | g1184771 | 92 | −75 | gb105pln | Zea mays glyceraldehyde-3-phosphate dehydrogenase GAPC2 (gpc2) mRNA, | |
| | | | | | complete cds. | |
| 700343770H1 | g1066931 | 17 | −3 | gb105eukp | F10B5.7 | |
| 700380569H1 | g829221 | 24 | −1 | gb105eukp | histone H2 A.F/Z | |
| 700346181H1 | g515376 | 48 | 14 | gb105pln | L. temulentum mRNA for histone H4. | |
| 700347212H1 | g2814799 | 31 | −17 | gb105eukp | ZK858.6 | |
| 700344430H1 | g21800 | 22 | −14 | gb105pln | T. aestivum L mRNA for histone H2B. | |
| 700343919H1 | g21800 | 50 | −63 | gb105pln | T. aestivum L mRNA for histone H2B. | |
| 700342824H1 | g22151 | 93 | −48 | gb105pln | Z. mays (A188) mRNA for alpha-tubulin 4. | |
| 700379868H1 | g1498365 | 46 | 9 | gb105pln | Solanum lycopersicum actin (Tom51) gene, partial cds. | |
| 700342776H1 | g758354 | 33 | 9 | gb105pln | Z. mays mRNA for plasma membrane H+ ATPase. | |
| 700342855H1 | g168498 | 32 | 16 | gb105pln | Corn histone H4 (H4C13) gene, complete cds. | |
| 700343154H1 | g2408013 | 29 | −18 | gb105pln | Yeast (Saccharomyces pombe) chromosome I cosmid c17G6. | |
| 700380479H1 | g517221 | 14 | 8 | gb105allp | ribosomal protein S24 | |
| 700380294H1 | g2244827 | 46 | −21 | gb105eukp | hypothetical protein | |
| 700347241H1 | g416148 | 71 | −69 | gb105pln | Zea mays beta-7 tubulin (tub7) mRNA, complete cds. | |
| 700345736H1 | g166857 | 58 | −60 | gb105pln | Arabidopsis thaliana cytoplasmic ribosomal protein mRNA, complete cds. | |
| 700347378H1 | g312500 | 46 | −28 | gb105eukp | 60S ribosomal protein L17 | |
| 700344146H1 | g1064828 | 7 | 2 | gb105eukp | Actin; Pharbitis nil actin 3-1 | |
| 700343356H1 | g312180 | 42 | −78 | gb105pln | Z. mays GapC4 gene. | |
| 700345514H1 | g2632928 | 15 | −3 | gb105allp | sorbitol dehydrogenase | |
| 700344064H1 | g1132482 | 65 | −64 | gb105pln | Rice mRNA for ADP-ribosylation factor, complete cds. | |
| 700343076H1 | g473933 | 6 | 6 | gb105allp | KIAA0052 | |
| 700347058H1 | g22322 | 15 | −38 | gb105pln | Z. mays mRNA for H2B histone (clone cH2B214). | |
| 700346972H1 | g929917 | 25 | −7 | gb105pln | Z. mays ABP1 mRNA for actin-depolymerizing factor. | |
| 700345515H1 | g2194119 | 7 | 6 | gb105eukp | F20P5.5 | |
| 700345227H1 | g1247302 | 12 | 1 | gb105allp | GBSS gene product | |
| 700281924H2 | g293888 | 31 | −21 | gb105pln | Zea mays, glyceraldehyde-3-phosphate dehydrogenase mRNA, 3′ end (clone GAPC2). | |
| 700343559H1 | g498788 | 37 | −22 | gb105pln | S. tuberosum (Desiree) cycl II mRNA. for cytochrome c1. | |
| 700346887H1 | g1143864 | 27 | 8 | gb105allp | beta glucosidase | |
| 700344745H1 | g1015849 | 12 | −1 | gb105eukp | RPS5 | |
| 700379945H1 | g2662344 | 82 | −70 | gb105pln | Oryza sativa mRNA for EF-1 alpha, complete cds. | |
| 700346795H1 | g166548 | 35 | −17 | gb105pln | Avena sativa vacuolar H+-ATPase 16 kDa proteolipid subunit (vatp-P1) mRNA, | |
| | | | | | complete cds. | |
| 700344374H1 | g1575129 | 60 | −58 | gb105pln | Zea mays lumenal binding protein cBiPe3 mRNA, complete cds. | |
| 700344331H1 | g170687 | 22 | 2 | gb105pln | Triticum aestivum DNA binding protein (EmBP-1c) mRNA, partial cds. | |
| 700344117H1 | g2828180 | 19 | −31 | gb105pln | Arabidopsis thaliana genomic DNA, chromosome 5, P1 clone: MDK4, complete | |
| | | | | | sequence. | |
| 700347323H1 | g2245027 | 10 | 0 | gb105eukp | ribosomal protein | |
| 700343453H1 | g1053056 | 36 | −34 | gb105pln | Triticum aestivum histone H3 gene, partial cds, clone W1. | |
| 700346919H1 | g1497983 | 42 | −22 | gb105eukp | alien; putative thyroid receptro interacting protein | |
| 700345905H1 | g498907 | 22 | −3 | gb105pln | Pisum sativum ribosomal protein L34 homolog (RPL34) mRNA, complete cds. | |
| 700344662H1 | g22245 | 96 | −86 | gb105pln | Zea mays DNA for cin4 element (showing homology to reverse transcriptase). | |
| 700342309H1 | g1420100 | 9 | 5 | gb105eukp | ORF YOR007c | |
| 700342869H1 | g184556 | 9 | 3 | gb105allp | insulin-degrading enzyme | |
| 700346631H1 | g1737491 | 58 | −49 | gb105pln | Triticum aestivum poly(A)-binding protein (wheatpab) mRNA, complete cds. | |
| 700345325H1 | g437901 | 21 | −6 | gb105eukp | phosphoprotein phosphatase 2A 65 kDa regulatory subunit | |
| 700343904H1 | g1107484 | 26 | −6 | gb105pln | A. thaliana mRNA for 40S ribosomal protein S15. | |
| 700346109H1 | g1483246 | 20 | 2 | gb105eukp | C52E4.3 | |
| 700344766H1 | g292055 | 43 | −22 | gb105allp | helix-loop-helix phosphoprotein | |
| 700346748H1 | g2773153 | 14 | −1 | gb105pln | Oryza sativa abscisic acid- and stress-inducible protein (Asr1) mRNA, complete cds. | |
| 700344711H1 | g1638836 | 45 | −49 | gb105pln | H. vulgare mRNA for alpha-tubulin 2. | |
| 700344215H1 | g168496 | 49 | −20 | gb105pln | Maize (Zea mays) histone H3 gene (H3C4), complete cds. | |
| 700347311H1 | g1086831 | 13 | −3 | gb105eukp | F10E7.7 | |
| 700345807H1 | g182514 | 28 | 4 | gb105allp | ferritin light chain | |
| 700345758H1 | g21841 | 18 | 6 | gb105pln | T. aestivum mRNA for a proline-rich protein. | |
| 700379905H1 | g303739 | 26 | −28 | gb105pln | Pea mRNA for GTP-binding protein, complete cds. | |
| 700342841H1 | g1041039 | 10 | 15 | gb105pln | Yeast gene for proteasome subunit complete cds. | |
| 700345986H1 | g1256607 | 11 | 12 | gb105pln | Glycine max G protein beta subunit mRNA, complete cds. | |
| 700281870H2 | g1418316 | 20 | −24 | gb105pln | A. thaliana CER1-like gene (4200 bp) | |
| 700342382H1 | g2088753 | 9 | 7 | gb105eukp | C53H9.2 | |
| 700379779H1 | g1657766 | 81 | −48 | gb105pln | Zea mays retrotransposon Opie-2 5′ LTR, primer binding site, gag gene, pol gene, | |
| | | | | | complete cds, polypurine tract and 3′ LTR. | |
| 700345521H1 | g1173554 | 27 | −13 | gb105pln | Pisum sativum UDP-galactose-4-epimerase (galE) mRNA, complete cds. | |
| 700344095H1 | g1051257 | 62 | −56 | gb105pln | Hordeum vulgare vacuolar ATPase catalytic subunit mRNA, partial cds. | |
| 700342335H1 | g311302 | 22 | −2 | gb105pln | N. sylvestris mRNA. | |
| 700347072H1 | g1381148 | 14 | 6 | gb105allp | Hs-CUL-4A | |
| 700343263H1 | g435648 | 64 | −56 | gb105pln | Rice mRNA for gamma-Tip, complete cds. | |
| 700346290H1 | g20255 | 55 | −45 | gb105pln | O. sativa gene for heat shock protein 82 HSP82. | |
| 700346004H1 | g2661839 | 32 | −16 | gb105pln | Physcomitrella patens mRNA for adenosine kinase. | |
| 700347078H1 | g1546918 | 44 | −41 | gb105pln | Z. mays mRNA for translation initiation factor 5A. | |
| 700346544H1 | g2244857 | 32 | −16 | gb105eukp | hypothetical protein | |
| 700344335H1 | g577824 | 27 | −35 | gb105pln | Z. mays gene for H2B histone (gH2B3). | |
| 700343778H1 | g1524382 | 58 | −76 | gb105pln | Z. mays mRNA for EPSP-synthase. | |
| 700343685H1 | g805003 | 45 | −45 | gb105pln | O. sativa SG12 gene. | |
| 700342543H1 | g895693 | 14 | 3 | gb105allp | ribosomal protein S15a | |
| 700345894H1 | g602252 | 80 | −60 | gb105pln | Zea mays enolase (eno2) mRNA, complete cds. | |
| 700342944H1 | g1495214 | 15 | −2 | gb105eukp | L13441; L1341 protein | |
| 700346554H1 | g2645068 | 18 | −3 | gb105eukp | W09D10.1 | |
| 700346816H1 | g2621777 | 12 | 4 | gb105allp | stomatin-like protein | |
| 700346792H1 | g1777929 | 23 | −5 | gb105pln | Saccharum officinarum nucleoside diphosphate kinase (SoNDPK1) mRNA, | |
| | | | | | complete cds. | |
| 700344705H1 | g1488267 | 15 | −5 | gb105eukp | low affinity calcium antiporter CAX2 | |
| 700281842H2 | g2296279 | 18 | −5 | gb105allp | unnamed protein product | |
| 700343856H1 | g2655115 | 35 | −1 | gb105pln | Zea mays Ca-5 mRNA, 3′UTR. | |
| 700380481H1 | g2641976 | 15 | 2 | gb105allp | mGpilp | |
| 700346762H1 | g1881692 | 79 | −70 | gb105pln | Zea mays phosphoglucomutase mRNA, partial cds. | |
| 700342305H1 | g1218053 | 41 | −26 | gb105pln | Oryza sativa protein phosphatase 2A mRNA, complete cds. | |
| 700345460H1 | g1182048 | 41 | −18 | gb105eukp | nda4; Nda4 | |
| 700342763H1 | g2708482 | 16 | 3 | gb105allp | IAA25 | |
| 700380590H1 | g473602 | 47 | −42 | gb105pln | Zea mays W-22 histone H2A mRNA, complete cds. | |
| 700344988H1 | g2252634 | 25 | −13 | gb105eukp | T19D16.28 | |
| 700347087H1 | g2656031 | 29 | −14 | gb105pln | Arabidopsis thaliana genomic DNA, chromosome 5, P1 clone: MXC20. | |
| 700344667H1 | g386764 | 75 | 3 | gb105allp | hba2 alpha globin | |
| 700343429H1 | g473933 | 9 | 3 | gb105allp | KIAA0052 | |
| 700343562H1 | g1430979 | 18 | −6 | gb105eukp | NOP1 | |
| 700344119H1 | g168460 | 86 | −32 | gb105pln | Zea mays cyclophilin (CyP) mRNA, complete cds. | |
| 700343447H1 | g416265 | 25 | −7 | gb105pln | Rice mRNA for ribosomal protein A2, partial sequence. | |
| 700344736H1 | g396254 | 25 | −9 | gb105eukp | 40S ribosomal protein S5 | |
| 700344508H1 | g2145358 | 8 | −3 | gb105eukp | ATHB-9; transcription factor; HD-Zip protein | |
| 700343051H1 | g861323 | 29 | −16 | gb105eukp | T24H7.1 | |
| 700380168H1 | g1944402 | 31 | −13 | gb105pln | Zinnia elegans mRNA for cinnamyl alcohol dehydrogenase, partial cds. | |
| 700380194H1 | g1332386 | 24 | −5 | gb105allp | Rattus norvegicus ATPase subunit 6 | |
| 700343085H1 | g171399 | 44 | −18 | gb105eukp | DIN1; ribonucleotide reductase DNA damage-inducible regulatory subunit | |
| 700344426H1 | g182514 | 27 | −6 | gb105allp | ferritin light chain | |
| 700344644H1 | g313151 | 26 | 1 | gb105pln | A. thaliana ribosomal protein S15 mRNA, complete CDS. | |
| 700343886H1 | g2104525 | 34 | −16 | gb105eukp | T10M13.2; T10M13.2 | |
| 700343696H1 | g1785643 | 17 | 5 | gb105allp | dinG | |
| 700347328H1 | g459267 | 36 | −12 | gb105pln | Z. mays gene for HMG protein. | |
| 700344330H1 | g871812 | 25 | −7 | gb105eukp | ARA.KIN | |
| 700347028H1 | g886470 | 29 | −15 | gb105pln | C. roseus MetE mRNA for methionine synthase. | |
| 700346002H1 | g498739 | 50 | −52 | gb105pln | H. vulgare (pMaW22) mRNA for beta-keotacyl-ACP synthase. | |
| 700345768H1 | g2463566 | 98 | −84 | gb105pln | Zea mays mRNA for squalene synthase, complete cds. | |
| 700379874H1 | g1002380 | 12 | 1 | gb105allp | RRM-type RNA binding protein | |
| 700380267H1 | g2257755 | 91 | −83 | gb105pln | Zea mays nucleolar histone deacetylase HD2-p39 mRNA, complete cds. | |
| 700344950H1 | g18140 | 10 | −0 | gb105pln | C. rubrum mRNA for light-induced 34 kD protein. | |
| 700343677H1 | g21694 | 22 | 4 | gb105pln | T. aestivum mRNA 2 for cathepsin B (2077). | |
| 700344336H1 | g886739 | 69 | −55 | gb105pln | Z. mays histone H4 gene. | |
| 700380083H1 | g960288 | 43 | −6 | gb105pln | Ruta graveolens anthranilate synthase alphs-subunit mRNA, complete cds. | |
| 700342458H1 | g1465368 | 14 | 6 | gb105allp | bHLH protein | |
| 700343493H1 | g168419 | 95 | −8 | gb105pln | Maize (Z. mays) aldolase mRNA, complete cds. | |
| 700342785H1 | g18449 | 36 | 11 | gb105pln | F. ananassa 26s rRNA gene. | |
| 700342324H1 | g1212996 | 12 | 4 | gb105eukp | UDP-glucose pyrophosphorylase; EC 2.7.7.9 | |
| 700343340H1 | g2213424 | 38 | −22 | gb105pln | Citrus paradisi mRNA for hypothetical protein. | |
| 700343904H1 | g313151 | 25 | −5 | gb105pln | A. thaliana ribosomal protein S15 mRNA, complete CDS. | |
| 700344755H1 | g577818 | 64 | −74 | gb105pln | Z. mays gene for H2B histone (gH2B4). | |
| 700344872H1 | g974781 | 42 | −46 | gb105pln | C. blumei kinetoplast met gene for cobalamine-independent methionine synthase. | |
| 700346836H1 | g1149594 | 34 | −22 | gb105pln | B. napus mRNA for 1-acyl-sn-glycerol-3-phosphate acyltransferase. | |
| 700346313H1 | g35768 | 8 | 6 | gb105allp | polypirimidine tract binding protein | |
| 700380716H1 | g1817578 | 40 | −22 | gb105allp | alpha-globin 2 | |
| 700346327H1 | g476103 | 18 | 2 | gb105allp | mago nashi protein | |
| 700343023H1 | g2737925 | 24 | −1 | gb105pln | Arabidopsis thaliana fimbrin-like protein AtFim2 mRNA, partial cds. | |
| 700343419H1 | g473986 | 35 | −31 | gb105pln | Rice mRNA, partial homologous to histone H2B gene. | |
| 700342692H1 | g2224352 | 36 | −23 | gb105pln | Chlorella vulgaris C-27 chloroplast DNA, complete sequence. | |
| 700345540H1 | g544491 | 61 | −47 | gb105pln | Corn mRNA for elongation factor 1A. | |
| 700281930H2 | g886739 | 22 | −37 | gb105pln | Z. mays histone H4 gene. | |
| 700342204H1 | g2345153 | 27 | −14 | gb105pln | Zea mays ribosomal protein S4 (rps4) mRNA, complete cds. | |
| 700343163H1 | g2673911 | 12 | −2 | gb105eukp | T24P15.11; putative squamosa-promoter binding protein | |
| 700345701H1 | g432367 | 38 | −31 | gb105pln | Rice mRNA for elongation factor 1 beta, complete cds. | |
| 700343570H1 | g669028 | 34 | −6 | gb105eukp | F32A5.7 | |
| 700379913H1 | g2494106 | 26 | 8 | gb105pln | Arabidopsis thaliana chromosome 1 YAC yUP8H12R, complete sequence. | |
| 700343815H1 | g2160158 | 41 | −16 | gb105eukp | F21M12.3 | |
| 700347124H1 | g971283 | 20 | −24 | gb105pln | Rice mRNA for ribosomal protein S31, complete cds. | |
| 700380064H1 | g397400 | 38 | −22 | gb105pln | B. rapa mRNA for S phase specific gene. | |
| 700343480H1 | g699510 | 12 | 4 | gb105allp | minute(1)1B protein | |
| 700379994H1 | g1399567 | 43 | −60 | gb105pln | Podophyllum peltatum nuclear 26S ribosomal RNA gene, partial sequence. | |
| 700346116H1 | g289212 | 18 | −17 | gb105pln | Atriplex nummularia homologous sequence (ANJ1) mRNA. | |
| 700346114H1 | g219910 | 28 | −10 | gb105allp | lipocortin II | |
| 700346952H1 | g218514 | 21 | 2 | gb105pln | Yeast (Saccharomyces cerevisiae) YS11A gene for ribosomal protein YS11. | |
| 700346325H1 | g2641200 | 44 | −31 | gb105pln | Fritillaria agrestis ribosomal protein L23a (rp123a) mRNA, complete cds. | |
| 700344974H1 | g21800 | 35 | −38 | gb105pln | T. aestivum L mRNA for histone H2B. | |
| 700345057H1 | g473602 | 64 | −59 | gb105pln | Zea mays W-22 histone H2A mRNA, complete cds. | |
| 700345019H1 | g496267 | 41 | −26 | gb105pln | Nicotiana tabacum GTP-binding protein (Ran-A1) mRNA, complete cds. | |
| 700379935H1 | g558366 | 73 | −74 | gb105pln | Z. mays CYP71C3 mRNA for cytochrome P-450. | |
| 700343381H1 | g1841307 | 11 | 17 | gb105pln | Fission Yeast mRNA for ribosomal protein S16 homolog, partial cds. | |
| 700346886H1 | g1841307 | 19 | 5 | gb105pln | Fission Yeast mRNA for ribosomal protein S16 homolog, partial cds. | |
| 700346584H1 | g2342688 | 23 | 1 | gb105eukp | F7G19.22 | |
| 700379995H1 | g695410 | 98 | −81 | gb105pln | Corn mRNA for glutamate dehydrogenase, complete cds. | |
| 700346682H1 | g2599103 | 21 | 2 | gb105pln | Dunaliella salina 60S ribosomal protein (DSRP1) mRNA, complete cds. | |
| 700342277H1 | g22322 | 21 | −35 | gb105pln | Z. mays mRNA for H2B histone (clone cH2B214). | |
| 700343927H1 | g984708 | 24 | −9 | gb105eukp | SPAC24H6.12c; unknown | |
| 700345614H1 | g19101 | 33 | −18 | gb105pln | H. vulgare mRNA for ribosomal protein L17-1. | |
| 700343915H1 | g1632831 | 11 | 3 | gb105allp | orf | |
| 700345093H1 | g2275007 | 76 | −69 | gb105pln | Hordeum vulgare mRNA for expressed sequence tag. | |
| 700346494H1 | g2342717 | 15 | 16 | gb105pln | Arabidopsis thaliana chromosome II BAC T14G11 genomic sequence, complete | |
| | | | | | sequence. | |
| 700346251H1 | g1149570 | 28 | −14 | gb105pln | A. thaliana mRNA for mitochondrial elongation factor Tu. | |
| 700343901H1 | g575730 | 81 | −78 | gb105pln | Z. mays mRNA for transmembrane protein. | |
| 700342389H1 | g170746 | 62 | −47 | gb105pln | Wheat histone H4 TH091 gene, complete cds. | |
| 700342789H1 | g22324 | 62 | −47 | gb105pln | Z. mays mRNA for H2B histone (clone cH2B221). | |
| 700344290H1 | g429021 | 49 | −51 | gb105pln | Rice mRNA for ribosomal protein S4 (gene name SS536), partial cds. | |
| 700345519H1 | g473985 | 45 | −34 | gb105pln | Rice mRNA, partial homologous to high mobility group protein gene. | |
| 700342379H1 | g290275 | 35 | −13 | gb105eukp | M(3)95A; multifunctional activity and location; ribosomal protein S3/AP endonuclease | |
| | | | | | DNA repair protein | |
| 700346145H1 | g487934 | 23 | −2 | gb105eukp | NAM8; Nam8p: Putative RNA binding proteins | |
| 700380796H1 | g780371 | 53 | −42 | gb105pln | Oryza sativa enolase mRNA, complete cds. | |
| 700281819H2 | g2088722 | 20 | −3 | gb105eukp | F53G12.10 | |
| 700342808H1 | g2529228 | 32 | −18 | gb105pln | Glycine max mRNA for 6-phosphogluconate dehydrogenase, complete cds. | |
| 700345469H1 | g2828287 | 9 | 6 | gb105eukp | T18B16.90; putative protein | |
| 700343627H1 | g303856 | 26 | 7 | gb105pln | Rice mRNA for ubiquitin protein fused to a ribosomal protein, complete cds. | |
| 700345645H1 | g461001 | 7 | 3 | gb105allp | NADPH-ferrihemoprotein reductase | |
| 700343207H1 | g1230639 | 12 | 6 | gb105eukp | YDR266C; Ydr266cp | |
| 700347264H1 | g2330705 | 7 | 6 | gb105eukp | SPAC16E8.17c; succinyl coA ligase | |
| 700343578H1 | g1518539 | 35 | −20 | gb105pln | Glycine max UDP-glucose dehydrogenase mRNA, complete cds. | |
| 700346916H1 | g1931639 | 17 | −1 | gb105eukp | T19D16.3; lysophospholipase isolog | |
| 700346303H1 | g2459406 | 21 | 3 | gb105pln | Arabidopsis thaliana chromosome II BAC F4P9 genomic sequence, complete sequence. | |
| 700345842H1 | g1931641 | 16 | 4 | gb105eukp | T19D16.5 | |
| 700380481H1 | g2623160 | 15 | 2 | gb105allp | N-acetylglucosamyl transferase component Gpi1 | |
| 700342961H1 | g2370511 | 21 | −8 | gb105eukp | SPAC2C6.14c; putative 40s ribosomal protein | |
| 700343483H1 | g1263302 | 51 | −8 | gb105eukp | pyruvate dehydrogenase E1 alpha subunit; EC 1.2.4.1 | |
| 700346257H1 | g507770 | 14 | 15 | gb105pln | Zea mays D3L H(+)-transporting ATPase (Mha1) gene, complete cds. | |
| 700281923H2 | g22322 | 15 | −42 | gb105pln | Z. mays mRNA for H2B histone (clone cH2B214). | |
| 700342668H1 | g19101 | 39 | −26 | gb105pln | H. vulgare mRNA for ribosomal protein L17-1. | |
| 700343828H1 | g1754992 | 65 | −51 | gb105pln | Triticum aestivum calmodulin TaCaM1-2 mRNA, complete cds. | |
| 700379825H1 | g473602 | 87 | −81 | gb105pln | Zea mays W-22 histone H2A mRNA, complete cds. | |
| 700281805H2 | g1906825 | 48 | −40 | gb105pln | A. thaliana hsp81.2 gene. | |
| 700342280H1 | g2668739 | 71 | −78 | gb105pln | Zea mays translation initiation factor GOS2 (TIF) mRNA, complete cds. | |
| 700345819H1 | g22483 | 35 | −31 | gb105pln | Z. mays RNA for superoxide dismutase Sod4. | |
| 700346896H1 | g169820 | 32 | −17 | gb105pln | Oryza sativa triosephosphate isomerase (Rictpi) mRNA, complete cds. | |
| 700345364H1 | g1129084 | 40 | −23 | gb105pln | Wheat mRNA for protein H2A, complete cds, clone wcH2A-3. | |
| 700347350H1 | g2599103 | 18 | 0 | gb105pln | Dunaliella salina 60S ribosomal protein (DSRP1) mRNA, complete cds. | |
| 700380047H1 | g763213 | 35 | −5 | gb105eukp | unknown | |
| 700379967H1 | g577824 | 35 | −42 | gb105pln | Z. mays gene for H2B histone (gH2B3). | |
| 700380121H1 | g473604 | 26 | −25 | gb105pln | Zea mays W-22 histone H2B mRNA, complete cds. | |
| 700347327H1 | g2702273 | 8 | 7 | gb105allp | carrot B2 protein-like | |
| 700344155H1 | g2244857 | 30 | −30 | gb105eukp | hypothetical protein | |
| 700346116H1 | g1125690 | 21 | −17 | gb105pln | S. tuberosum mRNA for DnaJ protein. | |
| 700346005H1 | g63064 | 28 | 3 | gb105allp | ribosomal protein P1 (AA 1-114) | |
| 700380557H1 | g166689 | 16 | −11 | gb105pln | A. thaliana 3-deoxy-D-arabino-heptulosonate 7-phosphate synthase (DHS2) mRNA, | |
| | | | | | complete cds. | |
| 700343365H1 | g534915 | 23 | −1 | gb105pln | S. tuberosum (Desiree) ppa mRNA for soluble inorganic pyrophosphatase. | |
| 700346391H1 | g460990 | 34 | −15 | gb105pln | O. sativa (Arborio) Beta Tubulin mRNA, clone OSTB-16. | |
| 700342479H1 | g818886 | 86 | −39 | gb105allp | KAP | |
| 700346734H1 | g459440 | 46 | −41 | gb105pln | F. pringlei modA mRNA. | |
| 700346694H1 | g2827143 | 97 | −0 | gb105eukp | Ath-B; cellulose synthase catalytic subunit | |
| 700347270H1 | g577732 | 15 | −22 | gb105pln | A. thaliana PRL1 gene. | |
| 700342419H1 | g1841869 | 21 | 0 | gb105pln | Pimpinella brachycarpa elongation factor 1-beta (EF-1-beta) mRNA, complete cds. | |
| 700347059H1 | g2462265 | 32 | −5 | gb105eukp | mobilization of fat during seed germination; patatin-like protein | |
| 700342989H1 | g1546918 | 78 | −79 | gb105pln | Z. mays mRNA for translation initiation factor 5A. | |
| 700344396H1 | g1196896 | 23 | 2 | gb105pln | Glycine max acidic ribosomal protein P0 mRNA, complete cds. | |
| 700347389H1 | g2160155 | 27 | −6 | gb105pln | Sequence of BAC F21M12 from Arabidopsis thaliana chromosome 1, complete | |
| | | | | | sequence. | |
| 700342490H1 | g21794 | 51 | −49 | gb105pln | Wheat histone H4 gene. | |
| 700380235H1 | g2708531 | 13 | 10 | gb105pln | Nicotiana tabacum putative RNA binding protein (QRRBP-1) mRNA, partial cds. | |
| 700345248H1 | g2746333 | 13 | 4 | gb105allp | RING zinc finger protein | |
| 700347034H1 | g1806139 | 41 | −27 | gb105pln | M. sativa mRNA for cdc2 kinase homologue, cdc2MsC. | |
| 700342610H1 | g1181330 | 50 | −33 | gb105pln | Z. mays CNX mRNA. | |
| 700343015H1 | g2353172 | 37 | −22 | gb105pln | Arabidopsis thaliana sigma factor 2 (SIG2) mRNA, nuclear gene encoding chloroplast | |
| | | | | | protein, complete cds. | |
| 700380070H1 | g2558943 | 46 | −4 | gb105pln | Gossypium hirsutum histone 3 mRNA, complete cds. | |
| 700342481H1 | g1463121 | 16 | −8 | gb105eukp | RPS3; ribosomal protein S3 | |
| 700343859H1 | g2829889 | 32 | −14 | gb105eukp | F3I6.29 | |
| 700342713H1 | g2408056 | 12 | 14 | gb105pln | Yeast (Saccharomyces pombe) chromosome I cosmid c2F3. | |
| 700344871H1 | g1045497 | 9 | 7 | gb105allp | conserved ATPase domain protein 44 | |
| 700345312H1 | g2465927 | 18 | −7 | gb105eukp | RKF3; receptor-like serine/threonine kinase | |
| 700380462H1 | g1129084 | 27 | −27 | gb105pln | Wheat mRNA for protein H2A, complete cds, clone wcH2A-3. | |
| 700281876H2 | g495263 | 21 | −14 | gb105eukp | sec61; sec61 protein | |
| 700343010H1 | g1336053 | 7 | 2 | gb105eukp | chymotrypsin-like protease precursor | |
| 700344512H1 | g2262155 | 15 | 11 | gb105pln | DNA sequence of Arabidopsis thaliana BAC F5J6 from chromosome IV, complete | |
| | | | | | sequence. | |
| 700347031H1 | g533251 | 96 | −42 | gb105pln | Zea mays (clone pSM8) sucrose synthase 2 (Sus1) gene, complete cds. | |
| 700346629H1 | g1208548 | 16 | 9 | gb105pln | Pseudosuga menziesii cysteine protease pseudotzain (PM33cysP) mRNA, complete cds. | |
| 700343240H1 | g292443 | 39 | −9 | gb105allp | ribosomal protein S20 | |
| 700379677H1 | g168490 | 59 | −60 | gb105pln | Maize glutathione S-transferase (GST-I) mRNA, complete cds. | |
| 700343243H1 | g2244950 | 14 | 15 | gb105pln | Arabidopsis thaliana DNA chromosome 4, ESSA I contig fragment No. 5. | |
| 700343512H1 | g852057 | 10 | 7 | gb105allp | casein kinase I-epsilon | |
| 700343880H1 | g218099 | 40 | 16 | gb105pln | Rice mRNA for ribosomal protein S12 (320 gene), partial sequence. | |
| 700345344H1 | g488576 | 17 | 1 | gb105pln | Medicago sativa Regen S clone pH3c131 histone H3.2 mRNA, complete cds. | |
| 700347227H1 | g452714 | 16 | −10 | gb105eukp | unknown | |
| 700347171H1 | g563245 | 16 | 5 | gb105eukp | RTS2; Rts2p | |
| 700346631H1 | g398326 | 33 | −21 | gb105pln | A. phyllitidis PABP mRNA for poly(A)-binding protein. | |
| 700345983H1 | g1185553 | 34 | −65 | gb105pln | Zea mays glyceraldehyde-3-phosphate dehydrogenase (gpc2) gene, complete cds. | |
| 700347118H1 | g886739 | 54 | −28 | gb105pln | Z. mays histone H4 gene. | |
| 700345273H1 | g2565339 | 41 | −28 | gb105pln | Lupinus luteus ribosomal protein S14 (rps14) mRNA, complete cds. | |
| 700344389H1 | g736308 | 8 | 1 | gb105eukp | unknown | |
| 700344742H1 | g22237 | 93 | −84 | gb105pln | Maize mRNA for cytosolic GAPDH (GapC) glyceraldehyde-3-phosphate dehydrogenase. | |
| 700344402H1 | g2244798 | 15 | −1 | gb105eukp | hypothetical protein | |
| 700343027H1 | g2245073 | 17 | 13 | gb105pln | Arabidopsis thaliana DNA chromosome 4, ESSA I contig fragment No. 8. | |
| 700344411H1 | g168602 | 22 | −40 | gb105pln | Zea mays regulatory protein GR14-12 mRNA, complete cds. | |
| 700345841H1 | g488198 | 17 | −0 | gb105eukp | ZC155.1 | |
| 700346386H1 | g1008175 | 18 | −1 | gb105eukp | ORF YJL045w | |
| 700346207H1 | g1742885 | 9 | 6 | gb105allp | Soribtol dehydrogenase (EC 1.1.1.14) (L-iditol 2-dehydrogenase). | |
| 700346033H1 | g2696030 | 30 | −14 | gb105eukp | PAP1; poly A polymerase | |
| 700342701H1 | g2464875 | 11 | 4 | gb105eukp | patatin homolog | |
| 700343263H1 | g1200160 | 29 | −19 | gb105pln | T. gesneriana mRNA for tonoplast intrinsic protein. | |
| 700343549H1 | g1842188 | 77 | 6 | gb105allp | mitochondrial phosphate translocator | |
| 700343992H1 | g473602 | 66 | −35 | gb105pln | Zea mays W-22 histone H2A mRNA, complete cds. | |
| 700344053H1 | g1370169 | 56 | −37 | gb105pln | L. japonicus mRNA for small GTP-binding protein, RAB1E. | |
| 700379938H1 | g474001 | 36 | −22 | gb105pln | Rice mRNA, partial homologous to ribosomal protein L38 gene. | |
| 700346436H1 | g896063 | 36 | 5 | gb105allp | PMS2 | |
| 700281996H2 | g1513228 | 12 | 5 | gb105eukp | myo-inositol 1-phosphate synthase | |
| 700344822H1 | g1841461 | 25 | −23 | gb105pln | N. tabacum mRNA for elongation factor 2. | |
| 700344810H1 | g1749509 | 22 | −3 | gb105pln | Fission Yeast mRNA, partial cds. | |
| 700347017H1 | g2407799 | 21 | −0 | gb105pln | Arabidopsis thaliana mRNA for histone H2A.F/Z. | |
| 700344856H1 | g2244842 | 17 | −6 | gb105eukp | hypothetical protein | |
| 700281971H1 | g498908 | 43 | 4 | gb105eukp | RPL34; ribosomal protein L34 homolog | |
| 700347309H1 | g313027 | 28 | −5 | gb105eukp | rp138; ribosomal protein L38 | |
| 700346242H1 | g960356 | 27 | −4 | gb105pln | Barley mRNA for S-adenosylmethionine synthetase, complete cds. | |
| 700347371H1 | g606969 | 26 | −7 | gb105pln | Arabidopsis thaliana cytoplasmic ribosomal protein L18 mRNA, complete cds. | |
| 700342456H1 | g2258073 | 35 | −39 | gb105pln | Hordeum vulgare var. distichum soluble inorganic pyrophosphatas (Ipp) mRNA, | |
| | | | | | complete cds. | |
| 700380075H1 | g473986 | 22 | −28 | gb105pln | Rice mRNA, partial homologous to histone H2B gene. | |
| 700342519H1 | g167109 | 61 | −53 | gb105pln | Hordeum vulgare vacuolar ATPase B subunit mRNA, complete cds. | |
| 700380044H1 | g2459446 | 13 | 3 | gb105allp | putative cinnamoyl-CoA reductase | |
| 700345308H1 | g2828283 | 13 | 7 | gb105allp | hypothetical protein | |
| 700345438H1 | g1399276 | 43 | −30 | gb105pln | Arabidopsis thaliana calmodulin-domain protein kinase CDPK isoform 7 (CPK7) mRNA, | |
| | | | | | complete cds. | |
| 700342607H1 | g338267 | 35 | −5 | gb105allp | small nuclear RNA protein (snRNP E) | |
| 700347241H1 | g493724 | 63 | −59 | gb105pln | O. sativa mRNA for beta-tubulin. | |
| 700344960H1 | g886739 | 82 | −72 | gb105pln | Z. mays histone H4 gene. | |
| 700379754H1 | g1419684 | 24 | 3 | gb105pln | M. sativa mRNA for TCTP-like protein. | |
| 700344703H1 | g29383 | 54 | 7 | gb105allp | BBC1 gene product | |
| 700346388H1 | g22430 | 20 | 7 | gb105pln | Maize pseudo-Gpa2 pseudogene for glyceraldehyde-3-phosphate dehydrogenase subunit A. | |
| 700342357H1 | g474000 | 64 | −47 | gb105pln | Rice mRNA, partial homologous to ribosomal protein L37a gene. | |
| 700344031H1 | g1498589 | 58 | 5 | gb105allp | 20S proteasome alpha subunit PSMA5 | |
| 700347121H1 | g747879 | 9 | 16 | gb105pln | Yeast (Saccharomyces cerevisiae) chromosome IV cosmid 9727. | |
| 700344932H1 | g547391 | 30 | −14 | gb105eukp | TRAP | |
| 700345023H1 | g2641233 | 17 | −2 | gb105eukp | ACT1; actin | |
| 700346776H1 | g2760839 | 17 | 1 | gb105eukp | F18A8.10; putative receptor kinase | |
| 700342996H1 | g1741868 | 20 | −6 | gb105allp | SDF2 | |
| 700380248H1 | g2274990 | 58 | −57 | gb105pln | Hordeum vulgare mRNA for expressed sequence tag. | |
| 700345540H1 | g2662346 | 61 | −48 | gb105pln | Oryza sativa mRNA for EF-1 alpha, complete cds. | |
| 700342494H1 | g1573716 | 12 | 3 | gb105allp | ATP-dependent clp protease proteolytic component (clpP) | |
| 700343648H1 | g1750404 | 28 | −6 | gb105eukp | B0412.4 | |
| 700345457H1 | g939780 | 20 | −13 | gb105pln | Zea mays MADS-box protein (ZAG5) mRNA, complete cds. | |
| 700347174H1 | g2429086 | 43 | −29 | gb105pln | Hordeum vulgare lipoxygenase 2 (LoxC) mRNA, complete cds. | |
| 700346944H1 | g793879 | 30 | −11 | gb105eukp | UBA2; Uba2 protein | |
| 700344412H1 | g2264302 | 12 | 14 | gb105pln | Arabidopsis thaliana genomic DNA, chromosome 5, P1 clone: MAC12, complete | |
| | | | | | sequence. | |
| 700380480H1 | g170784 | 33 | 0 | gb105pln | Wheat ubiquitin carrier protein (UBC1) mRNA, complete cds. | |
| 700347215H1 | g960291 | 23 | −16 | gb105eukp | anthranilate synthase alpha subunit | |
| 700344482H1 | g2766713 | 57 | −6 | gb105eukp | CMT1; chromomethylase | |
| 700346831H1 | g668984 | 27 | −5 | gb105pln | S. tuberosum TYKY1 mRNA for NADE:ubiquinone oxidoreductase. | |
| 700343608H1 | g2832667 | 26 | −6 | gb105pln | Arabidopsis thaliana DNA chromosome 4, BAC clone T10I14 (ESSAII project). | |
| 700342245H1 | g2293568 | 25 | −11 | gb105eukp | HvB12D homolog | |
| 700344058H1 | g183885 | 78 | −36 | gb105allp | gamma-globin | |
| 700346972H1 | g1419367 | 22 | −4 | gb105pln | Z. mays ZmABP2 mRNA for actin depolymerizing factor. | |
| 700343312H1 | g1572719 | 46 | −18 | gb105eukp | PAST-1 | |
| 700343065H1 | g777757 | 35 | −39 | gb105pln | Saccarum hybrid (clone SCUBI561) polyubiquitin mRNA, complete cds. | |
| 700344738H1 | g2213536 | 20 | −5 | gb105eukp | DNA-binding protein PD1 | |
| 700347133H1 | g2058280 | 51 | 0 | gb105eukp | atran3 | |
| 700344504H1 | g2645166 | 26 | −5 | gb105pln | Oryza sativa mRNA, similar to ubiquitin conjugating enzyme. | |
| 700379620H1 | g2351061 | 17 | −27 | gb105pln | Arabidopsis thaliana genomic DNA, chromosome 5, P1 clone: MAF19. | |
| 700342726H1 | g836954 | 10 | 1 | gb105allp | No definition line found | |
| 700344251H1 | g2213615 | 14 | 4 | gb105eukp | F21J9.9 | |
| 700379736H1 | g168500 | 16 | −13 | gb105pln | Maize (Zea mays) histone H4 gene (H4C14), complete cds. | |
| 700346567H1 | g2832595 | 14 | 1 | gb105allp | dJ434P1.2 | |
| 700342329H1 | g515359 | 18 | 1 | gb105pln | S. tuberosum pPOM36II mRNA for 36 kDa porinII. | |
| 700346079H1 | g927571 | 56 | −43 | gb105pln | Z. mays mRNA for calreticulin precursor. | |
| 700380256H1 | g1705482 | 17 | 1 | gb105eukp | F08B4.7 | |
| 700343631H1 | g456211 | 28 | −9 | gb105pln | S. tuberosum mRNA for 60S ribosomal protein L27. | |
| 700345415H1 | g218088 | 69 | −75 | gb105pln | Rice mRNA for ribosomal protein 117 (249 gene), partial sequence. | |
| 700343178H1 | g1082128 | 24 | −3 | gb105eukp | F45E6.3 | |
| 700343964H1 | g218176 | 13 | 15 | gb105pln | Rice mRNA for 21 kd polypeptide. | |
| 700344036H1 | g533251 | 74 | −30 | gb105pln | Zea mays (clone pSM8) sucrose synthase 2 (Sus1) gene, complete cds. | |
| 700345046H1 | g2688829 | 39 | −26 | gb105pln | Prunus armeniaca putative sugar transporter mRNA, complete cds. | |
| 700345632H1 | g463251 | 40 | −25 | gb105pln | M. sativa (Nagyszenasi) mRNA for ribosomal protein RL5. | |
| 700346429H1 | g2661839 | 21 | 3 | gb105pln | Physcomitrella patens mRNA for adenosine kinase. | |
| 700281884H2 | g717080 | 21 | 11 | gb105pln | Zea mays glyceraldehyde-3-phosphate dehydrogenase (GapC4) gene, promoter region. | |
| 700342910H1 | g1263468 | 13 | 3 | gb105eukp | F57A8.2 | |
| 700342979H1 | g2511596 | 14 | 8 | gb105eukp | prce; multicatalytic endopeptidase complex, proteasome precursor, beta subunit; | |
| | | | | | EC 3.4.99.46 | |
| 700346459H1 | g21794 | 53 | −50 | gb105pln | Wheat histone H4 gene. | |
| 700346437H1 | g20237 | 37 | −21 | gb105pln | O. sativa (rice) constitutive GOS2 gene. | |
| 700346387H1 | g2264309 | 15 | 14 | gb105pln | Arabidopsis thaliana genomic DNA, chromosome 5, P1 clone: MJJ3, complete sequence. | |
| 700347350H1 | g20163 | 40 | −29 | gb105pln | O. sativa Rr15 mRNA for 5S ribosomal RNA. | |
| 700343523H1 | g435679 | 36 | −11 | gb105eukp | ribosomal protein S25 | |
| 700342822H1 | g1262453 | 40 | −21 | gb105eukp | TpCCTeta; CCTeta | |
| 700344768H1 | g1149571 | 31 | −1 | gb105eukp | mitochondrial elongation factor Tu | |
| 700342789H1 | g577824 | 35 | −35 | gb105pln | Z. mays gene for H2B histone (gH2B3). | |
| 700342985H1 | g2529339 | 31 | −20 | gb105pln | Zea maize AGAMOUS homologue mRNA, complete cds. | |
| 700345118H1 | g624956 | 26 | −15 | gb105pln | C. reinhardtii mRNA for ribosomal protein S18. | |
| 700379869H1 | g2274990 | 72 | −68 | gb105pln | Hordeum vulgare mRNA for expressed sequence tag. | |
| 700345902H1 | g1136786 | 20 | −17 | gb105pln | Fission Yeast mRNA for elongation factor 1 alpha-C, complete cds. | |
| 700380756H1 | g485376 | 35 | −2 | gb105pln | Zea mays alpha-3-tubulin gene, complete cds. | |
| 700346694H1 | g2827139 | 82 | 2 | gb105eukp | RSW1; cellulos synthase catalytic subunit | |
| 700343245H1 | g927642 | 30 | −25 | gb105pln | Hordeum vulgare mRNA for betain aldehyde dehydrogenase, complete cds. | |
| 700345325H1 | g1568511 | 20 | −7 | gb105eukp | 65 kD regulatory subunit; protein phosphatase 2A | |
| 700379944H1 | g170455 | 63 | −54 | gb105pln | Tomato heat shock cognate protein 80 gene, 3′ end. | |
| 700344784H1 | g1248382 | 14 | 7 | gb105allp | subtilisin | |
| 700342952H1 | g1448967 | 16 | 0 | gb105allp | CROC-1B gene product | |
| 700344631H1 | g2196454 | 18 | −0 | gb105pln | Tobacco mRNA for A-type cyclin, complete cds. | |
| 700342526H1 | g2464904 | 27 | −4 | gb105eukp | X-Pro aminopeptidase homolog | |
| 700344625H1 | g606430 | 8 | 7 | gb105eukp | unknown | |
| 700344740H1 | g2529665 | 13 | −6 | gb105eukp | T30B22.8; putative ribosomal protein L7A | |
| 700345314H1 | g886739 | 47 | −70 | gb105pln | Z. mays histone H4 gene. | |
| 700380794H1 | g2264313 | 22 | −2 | gb105pln | Arabidopsis thaliana genomic DNA, chromosome 5, P1 clone: MOP10, complete | |
| | | | | | sequence. | |
| 700346689H1 | g603221 | 44 | −8 | gb105eukp | 6-phosphogluconate dehydrogenase | |
| 700347212H1 | g2435604 | 44 | −25 | gb105eukp | F08F1.7 | |
| 700380490H1 | g902524 | 31 | −23 | gb105pln | Zea mays clone MubG10 ubiquitin fusion protein gene, complete cds. | |
| 700343980H1 | g577824 | 40 | −43 | gb105pln | Z. mays gene for H2B histone (gH2B3). | |
| 700345113H1 | g1841421 | 17 | 2 | gb105eukp | S20 ribosomal protein | |
| 700342763H1 | g2618731 | 16 | 3 | gb105eukp | IAA21; IAA21 | |
| 700344952H1 | g21834 | 54 | −49 | gb105pln | Wheat mRNA for cytosolic phosphoglycerate kinase (EC 2.7.2.3). | |
| 700346512H1 | g1200039 | 9 | 7 | gb105eukp | F57C7.2 | |
| 700343258H1 | g1279640 | 7 | 5 | gb105eukp | NAM; apical meristem formation | |
| 700344336H1 | g170746 | 71 | −57 | gb105pln | Wheat histone H4 TH091 gene, complete cds. | |
| 700346985H1 | g168502 | 48 | −45 | gb105pln | Maize (Zea mays) histone H4 gene (H4C7), complete cds. | |
| 700343224H1 | g1931636 | 18 | −9 | gb105pln | Arabidopsis thaliana BAC T19D16 genomic sequence. | |
| 700281947H2 | g602605 | 35 | −48 | gb105pln | Zea mays Tandem genes for alpha1-tubulin and alpha2-tubulin. | |
| 700342639H1 | g17944 | 19 | −3 | gb105eukp | O-acetylserine (thiol)-lyase; EC 4.2.99.8 | |
| 700345250H1 | g21800 | 34 | −18 | gb105pln | T. aestivum L mRNA for histone H2B. | |
| 700344378H1 | g293888 | 70 | −81 | gb105pln | Zea mays, glyceraldehyde-3-phosphate dehydrogenase mRNA, 3′ end (clone GAPC2). | |
| 700343521H1 | g1513227 | 29 | −31 | gb105pln | Brassica napus myo-inositol 1-phosphate synthase mRNA, complete cds. | |
| 700345807H1 | g28519 | 32 | 2 | gb105allp | L apoferritin | |
| 700345511H1 | g601788 | 17 | 6 | gb105pln | Arabidopsis thaliana protein kinase (AFC2) mRNA, complete cds. | |
| 700380166H1 | g1816585 | 23 | −65 | gb105pln | Zea mays LON1 protease (LON1) mRNA, complete cds. | |
| 700343418H1 | g2341023 | 36 | −29 | gb105pln | Sequence of BAC F19P19 from Arabidopsis thaliana chromosome 1, complete sequence. | |
| 700343894H1 | g602605 | 71 | −42 | gb105pln | Zea mays tandem gene for alpha1-tubulin and alpha2-tubulin. | |
| 700347134H1 | g450292 | 51 | −55 | gb105pln | Zea mays alpha-tubulin mRNA, complete cds. | |
| 700345918H1 | g1620661 | 44 | −60 | gb105pln | Triticum aestivum 1,4-alpha-D-glucan 6-alpha-D-(1,4-alpha-D-glucanotransferase | |
| | | | | | mRNA, complete cds. | |
| 700347021H1 | g2827561 | 23 | 2 | gb105eukp | T12H17.230; predicted protein | |
| 700345905H1 | g436029 | 22 | −18 | gb105pln | Nicotiana tabacum (TSC40-3) 60S ribosomal protein L34 mRNA, complete cds. | |
| 700344205H1 | g2459406 | 26 | −16 | gb105pln | Arabidopsis thaliana chromosome II BAC F4P9 genomic sequence, complete sequence. | |
| 700345594H1 | g1184775 | 59 | −61 | gb105pln | Zea mays glyceraldehyde-3-phosphate dehydrogenase GAPC4 (gpc4) mRNA, | |
| | | | | | complete cds. | |
| 700281952H2 | g20250 | 41 | −41 | gb105pln | Oryza sativa H3 histone gene H3R-11. | |
| 700379889H1 | g974781 | 60 | −21 | gb105pln | C. blumei kinetoplast met gene for cobalamine-independent methionine synthase. | |
| 700343761H1 | g463856 | 30 | −15 | gb105pln | Chlamydomonas reinhardtii 21gr ribosomal protein S14 (CRY1) gene, complete cds. | |
| 700344745H1 | g396254 | 10 | −5 | gb105eukp | 40S ribosomal protein S5 | |
| 700346082H1 | g398921 | 43 | −32 | gb105pln | B. napus cold induced protein (BnC24B). | |
| 700344190H1 | g2078441 | 23 | −9 | gb105eukp | F52E4.7 | |
| 700346640H1 | g469248 | 25 | −11 | gb105eukp | ribosomal protein S3a | |
| 700342503H1 | g22340 | 66 | −72 | gb105pln | Maize gene for heat shock protein 70 exon 1 (hsp70; clone pMON 9502). | |
| 700346739H1 | g951334 | 9 | −3 | gb105eukp | LTP2; lipid transfer protein precursor | |
| 700343119H1 | g940151 | 24 | 6 | gb105allp | TAFII31 | |
| 700347158H1 | g166548 | 44 | −23 | gb105pln | Avena sativa vacuolar H+-ATPase 16 kDa proteolipid subunit (vatp-P1) mRNA, | |
| | | | | | complete cds. | |
| 700344247H1 | g1498383 | 48 | −40 | gb105pln | Zea mays actin (Maz89) gene, partial cds. | |
| 700342287H1 | g1334468 | 8 | −4 | gb105eukp | ATPase 6 (AA 1-229); Author-given protein sequence is in conflict with the conceptual | |
| | | | | | translation | |
| 700342392H1 | g862479 | 46 | −36 | gb105pln | Glycine max valosin-containing protein mRNA, complete cds. | |
| 700379677H1 | g168486 | 37 | −42 | gb105pln | Maize glutathione S-transferse gene (GST-I), exon 1. | |
| 700347309H1 | g2289009 | 31 | −6 | gb105eukp | T01O24.20; ribosomal protein L38 isolog | |
| 700347078H1 | g218082 | 39 | −10 | gb105pln | Rice mRNA for initiation factor eIF-4D (225 gene), partial sequence. | |
| 700346477H1 | g2642157 | 14 | 6 | gb105eukp | T5I7.5; ankyrin-like protein | |
| 700343608H1 | g602367 | 15 | 7 | gb105pln | Yeast (Saccharomyces cerevisiae) chromosome V cosmids 9871, 8199, 9867, 9495 and | |
| | | | | | lambda clones 6693 and 5898. | |
| 700345803H1 | g1236950 | 41 | −26 | gb105pln | Glycine max nucleoside diphosphate kinase mRNA, complete cds. | |
| 700347028H1 | g1814402 | 34 | −15 | gb105pln | Mesembryanthemum crystallinum methionine synthase (MetE) mRNA, complete cds. | |
| 700344607H1 | g508700 | 12 | −2 | gb105pln | Filobasidiella neoformans actin gene, complete cds. | |
| 700342460H1 | g1164925 | 16 | −6 | gb105pln | O. sativa matrix association region. | |
| 700342960H1 | g190336 | 58 | −44 | gb105allp | protease E precursor | |
| 700343160H1 | g1236960 | 10 | 16 | gb105pln | Prunus serotina prunasin hydrolase precursor mRNA, complete cds. | |
| 700346225H1 | g1016087 | 28 | −16 | gb105eukp | clpPI; ATP-dependent protease subunit; ClpP1 protease subunit | |
| 700344979H1 | g746415 | 10 | 4 | gb105allp | I kappa BR | |
| 700346876H1 | g1917018 | 38 | −57 | gb105pln | Zea mays ribosomal protein S6 RPS6-1 (rps6-1) mRNA, complete cds. | |
| 700346147H1 | g2565339 | 37 | −19 | gb105pln | Lupinus luteus ribosomal protein S14 (rps14) mRNA, complete cds. | |
| 700345874H1 | g1049306 | 39 | −11 | gb105pln | Arabidopsis thaliana actin-2 mRNA, complete cds. | |
| 700343667H1 | g2668737 | 23 | −44 | gb105pln | Zea mays translation initiation factor 5A (TIF5A) mRNA, complete cds. | |
| 700343458H1 | g6815 | 31 | −11 | gb105eukp | R08D7.3 | |
| 700344145H1 | g1399563 | 44 | −41 | gb105pln | Hydrastis canadensis nuclear 26S ribosomal RNA gene, partial sequence. | |
| 700347081H1 | g303854 | 65 | −52 | gb105pln | Rice mRNA for ribosomal protein L7A, complete cds. | |
| 700345651H1 | g2642152 | 18 | 0 | gb105pln | Arabidopsis thaliana chromosome II BAC T5I7 genomic sequence, complete sequence. | |
| 700343627H1 | g347063 | 25 | 9 | gb105pln | Brassica rapa ubiquitin/ribosomal protein mRNA, complete cds. | |
| 700379753H1 | g2262135 | 16 | 13 | gb105pln | Arabidopsis thaliana BAC T10P11, complete sequence. | |
| 700345689H1 | g1420870 | 20 | −6 | gb105pln | S. tuberosum mRAN for inorganic phosphate transporter, StPT1. | |
| 700343101H1 | g1766045 | 35 | −14 | gb105pln | Arabidopsis thaliana NAD+ dependent isocitrate dehydrogenase subunit 1 (IDH1) | |
| | | | | | mRNA, complete cds. | |
| 700343032H1 | g968901 | 47 | −42 | gb105pln | Rice mRNA for ribosomal protein S8, complete cds. | |
| 700346940H1 | g2317910 | 35 | −16 | gb105eukp | T7I23.10; CER1 protein | |
| 700346487H1 | g601790 | 28 | −10 | gb105pln | Arabidopsis thaliana protein kinase (AFC3) mRNA, partial cds. | |
| 700345783H1 | g2326410 | 4 | 8 | gb105eukp | 87B1.k | |
| 700346426H1 | g493587 | 11 | 6 | gb105eukp | PDI; catalyze the formation of disulfide bonds; disulfide isomerase; EC 5.3.4.1 | |
| 700344014H1 | g18046 | 66 | −2 | gb105eukp | Clkr27; beta-ketoacyl-ACP reductase; EC 1.1.1.100 | |
| 700342989H1 | g218082 | 51 | −38 | gb105pln | Rice mRNA for initiation factor eIF-4D (225 gene), partial sequence. | |
| 700344294H1 | g600709 | 33 | −24 | gb105pln | Arabidopsis thaliana synaptobrevin-related protein (SAR1) mRNA, complete cds. | |
| 700345424H1 | g22174 | 29 | −3 | gb105pln | Maize Aux311 gene for auxin-binding protein. | |
| 700344390H1 | g2695862 | 23 | −1 | gb105pln | Pisum sativum mRNA for 3-deoxy-D-manno-2-octulosonate-8-phosphate synthase, | |
| | | | | | clone pPS1A. | |
| 700346344H1 | g2239260 | 18 | −5 | gb105eukp | CCR; cinnamoyl CoA reductase | |
| 700344667H1 | g28549 | 75 | 3 | gb105allp | alpha globin | |
| 700344971H1 | g1737491 | 39 | −31 | gb105pln | Triticum aestivum poly(A)-binding protein (wheatpab) mRNA, complete cds. | |
| 700343447H1 | g790507 | 61 | −44 | gb105pln | Z. mays mRNA for 60S acidic ribosomal protein. | |
| 700344822H1 | g167244 | 22 | −23 | gb105pln | Chlorella kessleri elongation factor 2 mRNA, complete cds. | |
| 700346529H1 | g1129084 | 29 | −16 | gb105pln | Wheat mRNA for protein H2A, complete cds, clone wcH2A-3. | |
| 700342650H1 | g577611 | 95 | −51 | gb105pln | Zea mays CRT1 gene for calcium-binding protein. | |
| 700343736H1 | g683501 | 30 | −20 | gb105pln | A. thaliana mRNA for 65 kDa regulatory subunit of protein phosphatase 2A. | |
| 700344653H1 | g473602 | 95 | −34 | gb105pln | Zea mays W-22 histone H2A mRNA, complete cds. | |
| 700345321H1 | g303852 | 49 | −37 | gb105pln | Rice mRNA for ribosomal protein L3, complete cds. | |
| 700379735H1 | g167244 | 27 | −14 | gb105pln | Chlorella kessleri elongation factor 2 mRNA, complete cds. | |
| 700346241H1 | g1216484 | 38 | −24 | gb105eukp | ADK1; dual specificity kinase 1 | |
| 700343285H1 | g2257742 | 20 | −0 | gb105pln | Arabidopsis thaliana lysine-sensitive aspartate kinase mRNA, complete cds. | |
| 700343303H1 | g22324 | 68 | −57 | gb105pln | Z. mays mRNA for H2B histone (clone cH2B221). | |
| 700347186H1 | g1518156 | 40 | −23 | gb105pln | Glycine max RecA/Rad51/DMC1-like protein mRNA , complete cds. | |
| 700346248H1 | g2266661 | 36 | −17 | gb105pln | Hordeum vulgare mRNA for 14-3-3 protein (Hv1433c). | |
| 700345685H1 | g2262137 | 13 | 3 | gb105eukp | T10P11.3; sulfate transporter protein | |
| 700343584H1 | g927571 | 47 | −48 | gb105pln | Z. mays mRNA for calreticulin precursor. | |
| 700343558H1 | g499111 | 28 | −28 | gb105pln | Flaveria bidentis clone pNDK-b nucleoside diphosphate kinase mRNA, complete cds. | |
| 700347230H1 | g2645163 | 19 | 14 | gb105pln | Oryza sativa mRNA, similar to ribosomal protein L26. | |
| 700345475H1 | g606970 | 21 | −26 | gb105eukp | cytoplasmic ribosomal protein L18 | |
| 700343352H1 | g2827559 | 18 | −2 | gb105eukp | T12H17.210; predicted protein | |
| 700343380H1 | g2102693 | 23 | −6 | gb105eukp | Frk2; fructokinase; EC 2.7.1.4 | |
| 700345453H1 | g2464909 | 7 | 4 | gb105eukp | SCARECROW homolog | |
| 700344831H1 | g19699 | 35 | −9 | gb105eukp | NeIF-4A3; nicotiana eukaryotic translation initiation factor 4A | |
| 700380484H1 | g304109 | 16 | 7 | gb105eukp | PAB2; poly(A)-binding protein | |
| 700346249H1 | g2392895 | 10 | 2 | gb105eukp | BRI1; brassinosteroid insensitive 1 | |
| 700342555H1 | g386764 | 11 | −19 | gb105allp | hba2 alpha globin | |
| 700342927H1 | g2832642 | 13 | 4 | gb105eukp | F28J12.30; putative protein | |
| 700346123H1 | g2832681 | 34 | −4 | gb105eukp | T10T14.170; putative protein | |
| 700346817H1 | g534915 | 26 | −7 | gb105pln | S. tuberosum (Desiree) ppa mRNA for soluble inorganic pyrophosphatase. | |
| 700345676H1 | g1330253 | 43 | −2 | gb105pln | Zea mays calcium-dependent protein kinase mRNA, complete cds. | |
| 700343657H1 | g940450 | 16 | 4 | gb105allp | HISTONE H2A VARIANT | |
| 700343322H1 | g1244652 | 12 | −3 | gb105pln | Zea mays copia-type retroelement PREM-2 gag gene, complete cds. | |
| 700343809H1 | g1498389 | 41 | −52 | gb105pln | Zea mays actin (Maz81) gene, partial cds. | |
| 700343050H1 | g861323 | 22 | −12 | gb105eukp | T24H7.1 | |
| 700343656H1 | g2462748 | 25 | −16 | gb105eukp | F8A5.29; putative Clathrin Coat Assembly protein | |
| 700343889H1 | g435542 | 81 | −72 | gb105pln | Z. mays mRNA for calmodulin. | |
| 700344633H1 | g485952 | 56 | −45 | gb105pln | O. sativa mRNA for glutaredoxin. | |
| 700347382H1 | g644491 | 96 | −83 | gb105pln | Corn mRNA for elongation factor 1A. | |
| 700342746H1 | g1498052 | 89 | −80 | gb105pln | Zea mays ribosomal protein S8 mRNA, complete cds. | |
| 700343433H1 | g458426 | 13 | 1 | gb105eukp | dihydrolipoamide acetyltransferase | |
| 700343461H1 | g31062 | 16 | 6 | gb105allp | Epstein-Barr virus small RNA associated protein | |
| 700344443H1 | g16086 | 13 | 14 | gb105pln | A. porrum dnaJ mRNA for DNA J protein (partial). | |
| 700345511H1 | g642133 | 23 | −1 | gb105pln | Arabidopsis thaliana AME3 mRNA for protein kinase, complete cds. | |
| 700344544H1 | g397395 | 77 | −68 | gb105pln | Z. mays MNB1b mRNA for DNA-binding protein. | |
| 700345657H1 | g2668737 | 98 | −41 | gb105pln | Zea mays translation initiation factor 5A (TIF5A) mRNA, complete cds. | |
| 700345706H1 | g169977 | 15 | 6 | gb105allp | malate dehydrogenase | |
| 700343379H1 | g1498315 | 8 | 8 | gb105eukp | IAP100 | |
| 700343564H1 | g2465428 | 8 | −1 | gb105eukp | JRG1.2; 32 kDa protein | |
| 700344237H1 | g2564051 | 28 | −4 | gb105pln | Arabidopsis thaliana genomic DNA, chromosome 5, P1 clone: MWD9, complete | |
| | | | | | sequence. | |
| 700346924H1 | g2624327 | 24 | −3 | gb105pln | Oryza sativa mRNA for glycine rich RNA-binding protein 2 (OsGRF2). | |
| 700346226H1 | g1321660 | 18 | −39 | gb105pln | Rice mRNA for ascorbate peroxidase, complete cds. | |
| 700342260H1 | g1458245 | 11 | 1 | gb105eukp | F54D11.1 | |
| 700345483H1 | g2239260 | 15 | −0 | gb105eukp | CCR; cinnamoyl CoA reductase | |
| 700347048H1 | g1185555 | 41 | −14 | gb105pln | Zea mays glyceraldehyde-3-phosphate dehydrogenase (gpc4) gene, partial cds. | |
| 700344231H1 | g170746 | 49 | −40 | gb105pln | Wheat histone H4 TH091 gene, complete cds. | |
| 700344619H1 | g166606 | 16 | 4 | gb105eukp | anthranilate synthase alpha subunit | |
| 700380614H1 | g2662340 | 78 | −77 | gb105pln | Oryza sativa mRNA for EF-1 alpha, complete cds. | |
| 700379858H1 | g2506064 | 9 | 5 | gb105eukp | stil+ | |
| 700345784H1 | g1813915 | 8 | 7 | gb105eukp | F26E4.1 | |
| 700342828H1 | g463856 | 35 | −21 | gb105pln | Chlamydomonas reinhardtii 21 gr ribosomal protein S14 (CRY1) gene, complete cds. | |
| 700346992H1 | g166844 | 40 | −16 | gb105eukp | ribonucleoprotein | |
| 700342458H1 | g1142619 | 15 | 6 | gb105eukp | PG1; transcription factor; phaseolin G-box binding protein PG1 | |
| 700342409H1 | g22324 | 54 | −37 | gb105pln | Z. mays mRNA for H2B histone (clone cH2B221). | |
| 700342724H1 | g2245110 | 10 | 7 | gb105eukp | hypothetical protein | |
| 700347334H1 | g2286110 | 23 | −21 | gb105pln | Oryza sativa MADS box protein (OsMADS7) mRNA, complete cds. | |
| 700346064H1 | g1854579 | 10 | 5 | gb105allp | Int-6 | |
| 700345912H1 | g1850765 | 20 | −2 | gb105pln | Fission Yeast mRNA for ribosomal protein S23 homolog, partial cds. | |
| 700344884H1 | g2440191 | 17 | −10 | gb105eukp | hypothetical protein | |
| 700346340H1 | g2190272 | 22 | −2 | gb105allp | L-allo-threonine aldolase | |
| 700343126H1 | g1305524 | 10 | 15 | gb105pln | Oryza sativa Wilms' tumor-related protein QM mRNA, partial cds. | |
| 700344667H1 | g386765 | 75 | 3 | gb105allp | hba1 alpha globin | |
| 700342481H1 | g1302158 | 16 | −8 | gb105eukp | RPS3 | |
| 700346105H1 | g1323744 | 12 | −0 | gb105eukp | lipoxygenase | |
| 700345802H1 | g602605 | 12 | 4 | gb105pln | Zea mays tandem genes for alpha1-tubulin and alpha2-tubulin. | |
| 700343009H1 | g536891 | 29 | −42 | gb105pln | Wheat mRNA for protein H2A, complete cds, clone wcH2A-4. | |
| 700344868H1 | g168490 | 48 | −53 | gb105pln | Maize glutathione S-transferase (GST-I) mRNA, complete cds. | |
| 700343424H1 | g463856 | 27 | −35 | gb105pln | Chlamydomonas reinhardtii 21gr ribosomal protein S14 (CRY1) gene, complete cds. | |
| 700347118H1 | g170746 | 31 | −6 | gb105pln | Wheat histone H4 TH091 gene, complete cds. | |
| 700344119H1 | g829147 | 95 | −39 | gb105pln | Z. mays gene for cyclophilin. | |
| 700345121H1 | g17593 | 13 | −0 | gb105allp | THIOREDOXINE | |
| 700342447H1 | g2264318 | 29 | −8 | gb105pln | Arabidopsis thaliana genomic DNA, chromosome 5, P1 clone: MUP24, complete | |
| | | | | | sequence. | |
| 700342321H1 | g1870026 | 25 | −10 | gb105allp | histidine triad nucleotide-binding protein (HINT) | |
| 700342594H1 | g398848 | 98 | −84 | gb105pln | Z. mays mRNA for beta 5 tubulin. | |
| 700343418H1 | g218088 | 50 | −61 | gb105pln | Rice mRNA for ribosomal protein 117 (249 gene), partial sequence. | |
| 700347169H1 | g17797 | 22 | −7 | gb105eukp | Bplo | |
| 700346044H1 | g871507 | 34 | −18 | gb105pln | P. sativum mRNA for small GTP-binding protein (clone pGTP13). | |
| 700345963H1 | g2276349 | 25 | −5 | gb105pln | Yeast (Saccharomyces pombe) chromosome II cosmid c30D10. | |
| 700345847H1 | g1877169 | 37 | −16 | gb105eukp | W08E3.3 | |
| 700342403H1 | g396266 | 9 | 3 | gb105allp | orfX gene product | |
| 700379957H1 | g1420686 | 14 | −1 | gb105eukp | RPL18B | |
| 700380574H1 | g1054795 | 34 | −47 | gb105pln | H. vulgare mRNA for transmembrane protein. | |
| 700347055H1 | g939784 | 41 | −25 | gb105pln | Zea mays MADS-box protein (ZAP1) mRNA, complete cds. | |
| 700346591H1 | g2058311 | 15 | −7 | gb105eukp | CCR; cinnamoyl-CoA reductase; EC 1.2.1.44 | |
| 700380108H1 | g2673869 | 24 | −7 | gb105pln | Antirrhinum majus mRNA for fimbriata-associated protein 2, partial. | |
| 700380456H1 | g1814400 | 20 | −20 | gb105pln | Mesembryanthemum crystallinum phosphoglucomutase (PGM) mRNA, complete cds. | |
| 700379978H1 | g1839188 | 28 | −8 | gb105eukp | RHD3; putative GTP-binding protein; root hair defective 3 | |
| 700344335H1 | g21800 | 21 | −35 | gb105pln | T. aestivum L mRNA for histone H2B. | |
| 700380480H1 | g2689243 | 32 | 3 | gb105allp | ubiquitin conjugating enzyme | |
| 700344041H1 | g2245109 | 9 | −2 | gb105eukp | carboxyl-terminal proteinase homolog | |
| 700344721H1 | g19538 | 20 | −8 | gb105pln | M. crystallinum mRNA for ribosomal protein YL16. | |
| 700342455H1 | g2645166 | 30 | −11 | gb105pln | Oryza sativa mRNA, similar to ubiquitin conjugating enzyme. | |
| 700346381H1 | g166887 | 20 | −5 | gb105pln | Arabidopsis thaliana protein kinase (TMK1) gene, complete cds. | |
| 700346208H1 | g602330 | 13 | 17 | gb105pln | A. thaliana mRNA for O-acetylserine lyase (AT. OAS. 7-4). | |
| 700343567H1 | g2809480 | 22 | −11 | gb105pln | Oryza sativa calmodulin (CaM2) mRNA, complete cds. | |
| 700343846H1 | g968901 | 47 | −34 | gb105pln | Rice mRNA for ribosomal protein S8, complete cds. | |
| 700342411H1 | g1841940 | 18 | −10 | gb105eukp | SUM1 | |
| 700380557H1 | g169474 | 14 | −8 | gb105pln | S. tuberosum 3-deoxy-D-arabino-heptulosonate 7-phosphate synthase (aro1) mRNA, | |
| | | | | | complete cds. | |
| 700342308H1 | g1498337 | 56 | −49 | gb105pln | Glycine max actin (Soy119) gene, partial cds. | |
| 700380209H1 | g1816592 | 17 | 2 | gb105eukp | TCTP; translationally controlled tumor protein | |
| 700347190H1 | g2244945 | 21 | −5 | gb105eukp | similar to HYPOTHETICAL MYELOID CELL LINE PROTEIN 6 (FRAGMENT) | |
| 700347103H1 | g1498052 | 62 | −78 | gb105pln | Zea mays ribosomal protein S8 mRNA, complete cds. | |
| 700346917H1 | g172235 | 12 | 3 | gb105eukp | PR12 | |
| 700346734H1 | g1785859 | 46 | −42 | gb105pln | Flaveria linearis NADP-malic enzyme mRNA, partial cds. | |
| 700379941H1 | g218241 | 22 | −35 | gb105pln | Rice mRNA for ribosomal protein L3 (T82 gene), partial sequence. | |
| 700343291H1 | g872116 | 13 | 4 | gb105allp | sti (stress inducible protein) | |
| 700347019H1 | g974781 | 53 | −45 | gb105pln | C. blumei kinetoplast met gene for cobalamine-independent methionine synthase. | |
| 700379826H1 | g1145694 | 30 | −25 | gb105pln | Arabidopsis thaliana actin (ACT3) gene, complete cds. | |
| 700343384H1 | g264277 | 71 | −53 | gb105pln | auxin-binding protein [corn, mRNA, 795 nt]. | |
| 700344325H1 | g1574748 | 12 | 5 | gb105allp | tRNA pseudouridine 55 synthase (truB) | |
| 700281965H1 | g2852446 | 20 | −4 | gb105pln | Arabidopsis thaliana APK2a mRNA for protein kinase, complete cds. | |
| 700380188H1 | g2828186 | 20 | 2 | gb105pln | Arabidopsis thaliana genomic DNA, chromosome 5, TAC clone: K18I23, complete | |
| | | | | | sequence. | |
| 700344882H1 | g902583 | 72 | −69 | gb105pln | Zea mays clone MubG1 ubiquitin gene, complete cds. | |
| 700344065H1 | g209383 | 46 | −4 | gb105allp | MS-2 pol-stefin B fusion protein | |
| 700346988H1 | g1149474 | 11 | −1 | gb105eukp | F22B3.4 | |
| 700343010H1 | g2853182 | 7 | 3 | gb105eukp | Digestive; Trypsin; EC 3.4.21.4 | |
| 700346201H1 | g22302 | 32 | −73 | gb105pln | Maize Gpc1 gene for glyceraldehyde-3-phosphate dehydrogenase (GADPH) subunit C. | |
| 700343059H1 | g439585 | 50 | −37 | gb105pln | Hordeum vulgare calreticulin (CRH1) mRNA, partial cds. | |
| 700345575H1 | g2827887 | 40 | −30 | gb105pln | Arabidopsis thaliana ATP-dependent Clp protease proeolytic subunit (clpP) mRNA, | |
| | | | | | nuclear gene encoding chloroplast protein, complete cds. | |
| 700380779H1 | g166548 | 41 | −31 | gb105pln | Avena sativa vacuolar H+-ATPase 16 kDa proteolipid subunit (vatp-P1) mRNA, | |
| | | | | | complete cds. | |
| 700347084H1 | g473604 | 33 | −28 | gb105pln | Zea mays W-22 histone H2B mRNA, complete cds. | |
| 700346090H1 | g1435156 | 37 | −41 | gb105pln | L. esculentum mRNA for histone H3 variant H3.3. | |
| 700345364H1 | g1129085 | 42 | −26 | gb105pln | Wheat mRNA for protein H2A, complete cds, clone wcH2A-9. | |
| 700343005H1 | g168460 | 83 | −67 | gb105pln | Zea mays cyclophilin (CyP) mRNA, complete cds. | |
| 700344675H1 | g3046 | 15 | 1 | gb105eukp | NUO-40; 40 kD subunit of NADH dehydrogenase | |
| 700380280H1 | g2702371 | 8 | 1 | gb105eukp | B0546.4 | |
| 700379777H1 | g2286154 | 41 | −37 | gb105pln | Zea mays 6-phosphofructo-2-kinase/fructose-2,6-bisphosphatase mRNA, partial cds. | |
| 700343655H1 | g2414643 | 30 | −12 | gb105pln | Yeasts (Saccharomyces pombe) chromosome I cosmid c3H5. | |
| 700379991H1 | g2407799 | 45 | −30 | gb105pln | Arabidopsis thaliana mRNA for histone H2A.F/Z. | |
| 700347360H1 | g1262279 | 20 | −10 | gb105eukp | MDL3; catalyzes dissociation of (R)-mandelonitrile to hydrogen cyanide and | |
| | | | | | benzaldehyde; (R)-(+)-mandelonitrile lyase isoform MDL3 precursor; EC 4.1.2.10 | |
| 700347174H1 | g2182266 | 60 | −46 | gb105pln | Hordeum vulgare lipoxygenase (LoxB) mRNA, complete cds. | |
| 700342429H1 | g1022806 | 23 | −5 | gb105pln | Arabidopsis thaliana cellulase (OR16pep) mRNA, complete cds. | |
| 700343495H1 | g2266661 | 22 | −6 | gb105pln | Hordeum vulgare mRNA for 14-3-3 protein (Hv1433c). | |
| 700346082H1 | g398917 | 44 | −34 | gb105pln | B. napus cold induced protein (BnC24A) mRNA. | |
| 700345362H1 | g2264307 | 16 | 5 | gb105pln | Arabidopsis thaliana genomic DNA, chromosome 5, P1 clone: MED24, complete | |
| | | | | | sequence. | |
| 700346640H1 | g2792508 | 33 | −24 | gb105eukp | ribosomal protein S3a | |
| 700343980H1 | g21800 | 23 | −47 | gb105pln | T. aestivum L mRNA for histone H2B. | |
| 700345233H1 | g2414643 | 17 | −12 | gb105pln | Yeast (Saccharomyces pombe) chromosome I cosmid c3H5. | |
| 700379825H1 | g1322276 | 25 | −15 | gb105pln | Triticum aestivum histone H2A gene, complete cds. | |
| 700347201H1 | g2330866 | 8 | 5 | gb105eukp | SPAC6G10.02c; hypothetical protein | |
| 700343386H1 | g1789489 | 13 | 6 | gb105allp | hypothetical 37.4 kD protein in exuR-tdcC intergenic region | |
| 700345458H1 | g170746 | 59 | −43 | gb105pln | Wheat histone H4 TH091 gene, complete cds. | |
| 700343461H1 | g1213516 | 18 | 3 | gb105allp | ribosomal protein L22 | |
| 700343250H1 | g487315 | 21 | −13 | gb105pln | Rice mRNA EN520, partial sequence. | |
| 700344522H1 | g303834 | 33 | −30 | gb105pln | Rice mRNA for 21 kd polypeptide, complete cds. | |
| 700380235H1 | g2708532 | 15 | 5 | gb105allp | putative RNA binding protein | |
| 700343461H1 | g409074 | 16 | 6 | gb105allp | HBp15/L22 | |
| 700342656H1 | g313151 | 22 | −4 | gb105pln | A. thaliana ribosomal protein S15 mRNA, complete CDS. | |
| 700347125H1 | g1155264 | 65 | −58 | gb105pln | Pennisetum ciliare possible apospory-associated protein mRNA, complete cds. | |
| 700346666H1 | g602605 | 69 | −76 | gb105pln | Zea mays tandem genes for alpha1-tubulin and alpha2-tubulin. | |
| 700344087H1 | g2623294 | 20 | −4 | gb105pln | Arabidopsis thaliana chromosome II BAC T20B5 genomic sequence, complete sequence. | |
| 700380462H1 | g1129085 | 26 | −25 | gb105pln | Wheat mRNA for protein H2A, complete cds, clone wcH2A-9. | |
| 700344352H1 | g20255 | 57 | −57 | gb105pln | O. sativa gene for heat shock protein 82 HSP82. | |
| 700346916H1 | g2529663 | 18 | −2 | gb105eukp | T30B22.6; putative lysophospholipase | |
| 700346709H1 | g1944506 | 45 | −40 | gb105pln | Spinacia oleracea mRNA for thylakoid-bound ascorbate peroxidase, complete cds. | |
| 700345687H1 | g1289304 | 14 | −0 | gb105eukp | TCP20; Tcp20p | |
| 700380146H1 | g786228 | 9 | 8 | gb105eukp | tRNA-Gly | |
| 700346419H1 | g454881 | 15 | 1 | gb105pln | Rice gene for thioredoxin h, complete cds. | |
| 700346110H1 | g2073547 | 24 | −11 | gb105pln | Arabidopsis thaliana polyubiquitin (ubq4) gene, complete cds. | |
| 700346939H1 | g1777929 | 50 | −55 | gb105pln | Saccharum officinarum nucleoside diphosphate kinase (SoNDPK1) mRNA, | |
| | | | | | complete cds. | |
| 700345984H1 | g168500 | 75 | −17 | gb105pln | Maize (Zea mays) histone H4 gene (H4C14), complete cds. | |
| 700379870H1 | g1200153 | 27 | −14 | gb105pln | S. tuberosum mRNA for starch synthase. | |
| 700342564H1 | g387908 | 19 | 3 | gb105pln | Brassica rapa S-phase-specific (BIS289) mRNA, complete cds. | |
| 700342652H1 | g2264309 | 28 | −2 | gb105pln | Arabidopsis thaliana genomic DNA, chromosome 5, P1 clone: MJJ3, complete sequence. | |
| 700343201H1 | g2252839 | 26 | −12 | gb105eukp | A_IG005I10.19 | |
| 700343416H1 | g1841467 | 20 | −22 | gb105pln | N. tabacum mRNA for tyrosyl-tRNA synthetase. | |
| 700344238H1 | g22206 | 40 | −20 | gb105pln | Maize Ins1 insertion element upstream of bronze gene Bz-McC. | |
| 700342236H1 | g1066996 | 45 | −23 | gb105eukp | K03H1.2 | |
| 700343652H1 | g471089 | 28 | −9 | gb105eukp | ACA1; chloroplast envelope Ca2+-ATPase precursor | |
| 700346952H1 | g1235570 | 22 | −1 | gb105pln | P. anserina su12 gene. | |
| 700345704H1 | g1668705 | 34 | −36 | gb105pln | A. thaliana mRNA for AtRan2 protein. | |
| 700342701H1 | g2464876 | 11 | 1 | gb105allp | patatin homolog | |
| 700380613H1 | g577824 | 24 | −25 | gb105pln | Z. mays gene for H2B histone (gH2B3). | |
| 700346227H1 | g2213594 | 18 | −6 | gb105eukp | T7N9.14 | |
| 700342455H1 | g456567 | 30 | −9 | gb105pln | Pisum sativum ubiquitin conjugating enzyme (UBC4), complete cds. | |
| 700343531H1 | g1296954 | 38 | −31 | gb105pln | O. sativa mRNA for novel protein, osr40c1. | |
| 700346066H1 | g22681 | 42 | −31 | gb105pln | L. usitatissimum mRNA for Stearoyl-(acyl-carrier-protein)desaturase. | |
| 700345314H1 | g170746 | 41 | −40 | gb105pln | Wheat histone H4 TH091 gene, complete cds. | |
| 700379940H1 | g2282583 | 72 | −62 | gb105pln | Zea mays elongation factor 1-alpha (EF1-A) mRNA, complete cds. | |
| 700345415H1 | g310932 | 47 | −48 | gb105pln | Nicotiana tabacum ribosomal protein L17 mRNA, complete cds. | |
| 700347201H1 | g644882 | 7 | 8 | gb105allp | HCF | |
| 700342353H1 | g2464911 | 42 | −20 | gb105eukp | splicing factor homolog | |
| 700379811H1 | g2198852 | 35 | −82 | gb105pln | Zea mays cystathionine gamma-synthase (CGS1) gene, complete cds. | |
| 700347078H1 | g2668737 | 48 | −57 | gb105pln | Zea mays translation initiation factor 5A (TIF5A) mRNA, complete cds. | |
| 700345951H1 | g388206 | 51 | −18 | gb105pln | Lycopersicon esculentum ubiquitin carrier protein (Ubc) mRNA, complete cds. | |
| 700346893H1 | g1632830 | 23 | 9 | gb105pln | R. communis mRNA (unknown). | |
| 700344417H1 | g19572 | 24 | −3 | gb105pln | M. sativa C29 mRNA for SnRNP-related protein. | |
| 700380115H1 | g2665672 | 16 | −4 | gb105eukp | IOTA; proteasome IOTA subunit | |
| 700346920H1 | g4038 | 22 | −13 | gb105pln | Yeast (Saccharomyces cerevisiae) DNA for 38 kd nukleolar protein NOP1. | |
| 700380647H1 | g871985 | 67 | −57 | gb105pln | A. sativa Aspk11 mRNA. | |
| 700347107H1 | g391874 | 34 | −19 | gb105pln | Rice mRNA for 40S subunit ribosomal protein, complete cds. | |
| 700345885H1 | g218088 | 52 | −42 | gb105pln | Rice mRNA for ribosomal protein 117 (249 gene), partial sequence. | |
| 700344393H1 | g1145694 | 26 | −18 | gb105pln | Arabidopsis thaliana actin (ACT3) gene, complete cds. | |
| 700345072H1 | g2827080 | 9 | 7 | gb105eukp | mmdh; malate dehydrogenase precursor; EC 1.1.1.37 | |
| 700343122H1 | g248336 | 73 | −57 | gb105pln | polyubiquitin [maize, Genomic, 3841 nt]. | |
| 700380781H1 | g22324 | 33 | −50 | gb105pln | Z. mays mRNA for H2B histone (clone cH2B221). | |
| 700379927H1 | g166857 | 41 | −29 | gb105pln | Arabidopsis thaliana cytoplasmic ribosomal protein mRNA, complete cds. | |
| 700379637H1 | g2673867 | 28 | −9 | gb105pln | Antirrhinum majus mRNA for fimbriate-associated protein 1, partial. | |
| 700281992H2 | g2696803 | 20 | −33 | gb105pln | Oryza sativa mRNA for water channel protein, complete cds. | |
| 700380074H1 | g498772 | 45 | −45 | gb105pln | Z. mays (cv DH5xDH7) hsp70-4 mRNA for heat shock protein 70. | |
| 700379877H1 | g1397319 | 15 | 0 | gb105eukp | R08C7.2 | |
| 700343417H1 | g1053092 | 16 | 9 | gb105pln | Arabidopsis thaliana pre zeta-carotene desaturase precursor (zds) mRNA, | |
| | | | | | completecds. | |
| 700344091H1 | g556685 | 33 | −13 | gb105pln | Z. mays mRNA for ADP-ribosylation factor. | |
| 700345951H1 | g441456 | 51 | −18 | gb105pln | L. esculentum mRNA for ubiquitin conjugating enzyme E2. | |
| 700345605H1 | g8040 | 23 | −3 | gb105eukp | histone H2A variant (AA 1-141) | |
| 700345475H1 | g1550725 | 14 | −14 | gb105eukp | ribosomal protein L18 | |
| 700345620H1 | g467751 | 5 | 6 | gb105allp | 2-phosphoglycerate kinase | |
| 700346859H1 | g1015849 | 14 | −2 | gb105eukp | RPS5 | |
| 700347223H1 | g2660661 | 13 | 15 | gb105pln | Arabidopsis thaliana BAC T19K24 genomic sequence, complete sequence. | |
| 700342379H1 | g1055070 | 37 | −17 | gb105eukp | C23G10.3 | |
| 700281918H2 | g576505 | 16 | 3 | gb105eukp | IAP34; component of chloroplast outer membrane protein import apparatus; GTP-binding | |
| | | | | | protein | |
| 700346456H1 | g169818 | 18 | 14 | gb105pln | Rice 25S ribosomal RNA gene. | |
| 700345819H1 | g22484 | 42 | −31 | gb105pln | Z. mays RNA for superoxide dismutase Sod4A. | |
| 700345524H1 | g520543 | 46 | −45 | gb105pln | Sorghum bicolor clone BADH1 betaine aldehyde dehydrogenase mRNA, partial cds. | |
| 700343843H1 | g1495339 | 31 | −12 | gb105eukp | F26A3.3 | |
| 700380076H1 | g2351580 | 50 | −3 | gb105eukp | thymidine diphospho-glucose 4-6-dehydratase homolog | |
| 700379964H1 | g2558668 | 22 | 8 | gb105allp | eukaryotic translation initiation factor | |
| 700344509H1 | g500850 | 54 | −42 | gb105pln | Zea mays (clone pAKHSDH1) aspartate kinase-homoserine dehydrogenase mRNA, | |
| | | | | | complete cds. | |
| 700344755H1 | g21800 | 35 | −54 | gb105pln | T. aestivum L mRNA for histone H2B. | |
| 700281881H2 | g1617206 | 26 | −1 | CP12 | | |
| 700343909H1 | g1125690 | 30 | −14 | gb105pln | S. tuberosum mRNA for DnaJ protein. | |
| 700346373H1 | g1890574 | 24 | −25 | gb105pln | H. vulgare mRNA for xyloglucan endotransglycosylase-like protein (XEA). | |
| 700346145H1 | g2708532 | 22 | −3 | gb105eukp | QRRBP-1; putative RNA binding protein | |
| 700344482H1 | g2766714 | 31 | 2 | gb105pln | Arabidopsis arenosa isolate Care-1 (9509) chromomethylase (CMT1) mRNA, partial cds. | |
| 700346619H1 | g2505873 | 22 | −0 | gb105pln | Arabidopsis thaliana DNA, 40 kb surrounding ACS1 locus. | |
| 700342573H1 | g1514583 | 53 | −37 | gb105allp | immunoglobulin kappa light chain | |
| 700343656H1 | g451888 | 16 | −10 | gb105eukp | | |
| 700344177H1 | g2827714 | 8 | 4 | gb105allp | receptor protein kinase-like protein | |
| 700344971H1 | g2213870 | 23 | −3 | gb105pln | Mesembryanthemum crystallinum poly(A)-binding protein mRNA, partial cds. | |
| 700346226H1 | g2274983 | 16 | −35 | gb105pln | Hordeum vulgare mRNA for expressed sequence tag. | |
| 700345581H1 | g416256 | 22 | −18 | gb105pln | Rice mRNA for H+-ATPase, partial sequence. | |
| 700379827H1 | g535587 | 52 | −48 | gb105pln | Arabidopsis thaliana chaperone protein (atj) mRNA, complete cds. | |
| 700344409H1 | g1498052 | 34 | −52 | gb105pln | Zea mays ribosomal protein S8 mRNA, complete cds. | |
| 700347105H1 | g2244837 | 34 | −8 | gb105eukp | proteasome chain protein | |
| 700343409H1 | g19698 | 53 | −45 | gb105pln | N. plubaginifolia NeIF-4A3 mRNA for nicotiana eukaryotic translation initiation factor | |
| | | | | | 4A. | |
| 700344766H1 | g1911237 | 34 | −17 | gb105allp | G protein signaling regulator RGS2 | |
| 700345906H1 | g2792216 | 9 | 5 | gb105eukp | b8; NBS-LRR type resistance protein | |
| 700344038H1 | g57117 | 20 | 5 | gb105allp | ribosomal protein L32 | |
| 700345535H1 | g1082107 | 8 | 6 | gb105eukp | F17E5.2 | |
| 700342788H1 | g246 | 9 | 1 | gb105allp | NADH dehydrogenase | |
| 700344004H1 | g2642214 | 34 | 5 | gb105pln | Arabidopsis thaliana NOI protein mRNA, complete cds. | |
| 700344868H1 | g168486 | 35 | −42 | gb105pln | Maize glutathione S-transferase gene (GST-I), exon 1. | |
| 700343950H1 | g552061 | 20 | 4 | gb105eukp | actin | |
| 700344322H1 | g166409 | 16 | 9 | gb105pln | Alfalfa nucleic acid binding protein (alfin-1) mRNA, partial cds. | |
| 700345779H1 | g1015699 | 6 | 7 | gb105allp | ORF YJR044c | |
| 700343491H1 | g602605 | 44 | 13 | gb105pln | Zea mays tandem genes for alpha1-tubulin and alpha2-tubulin. | |
| 700343391H1 | g21624 | 40 | −24 | gb105pln | S. vulgare mRNA for glycine-rich RNA-binding protein (clone S2). | |
| 700342691H1 | g168490 | 93 | −78 | gb105pln | Maize glutathione S-transferase (GST-I) mRNA, complete cds. | |
| 700344278H1 | g22483 | 57 | −57 | gb105pln | Z. mays RNA for superoxide dismutase Sod4. | |
| 700380714H1 | g1322276 | 24 | −5 | gb105pln | Triticum aestivum histone H2A gene, complete cds. | |
| 700347383H1 | g1388076 | 21 | −2 | gb105eukp | TRX3; thioredoxin h | |
| 700344496H1 | g1469041 | 10 | 5 | gb105eukp | F23B12.5 | |
| 700345009H1 | g939784 | 40 | −24 | gb105pln | Zea mays MADS-box protein (ZAP1) mRNA, complete cds. | |
| 700344734H1 | g1209700 | 70 | −72 | gb105pln | Zea mays ribosomal protein L12 mRNA, complete cds. | |
| 700345174H1 | g2632254 | 12 | 2 | gb105eukp | SNFL2; serine/threonine kinase | |
| 700281821H2 | g248338 | 80 | −77 | gb105pln | polyubiquitin [maize, Genomic, 3439 nt]. | |
| 700343687H1 | g17958 | 34 | −18 | gb105pln | C. cardunculus cypro4 mRNA. | |
| 700344173H1 | g1184771 | 80 | −73 | gb105pln | Zea mays glyceraldehyde-3-phosphate dehydrogenase GAPC2 (gpc2) mRNA, | |
| | | | | | complete cds. | |
| 700380480H1 | g170785 | 33 | 3 | gb105eukp | UBC1; ubiquitin carrier protein | |
| 700346487H1 | g642131 | 19 | 3 | gb105pln | Arabidopsis thaliana AME2 mRNA for protein kinase, complete cds. | |
| 700345761H1 | g1706955 | 48 | −14 | gb105pln | Gossypium hirsutum cellulose synthase (celA1) mRNA, complete cds. | |
| 700342580H1 | g22328 | 50 | −33 | gb105pln | Maize mRNA for a high mobility group protein. | |
| 700346571H1 | g410285 | 26 | −11 | gb105pln | Pisum sativum rho (ras-related) GTP-binding protein mRNA, complete cds. | |
| 700346414H1 | g984964 | 7 | 4 | gb105eukp | SIK1; suppressor of toxicity of GAL4-IKB; Sik1p | |
| 700343641H1 | g168462 | 13 | 12 | gb105pln | Zea mays defective suppressor-mutator from the brittle-1-mutable allele. | |
| 700346440H1 | g2459406 | 35 | −37 | gb105pln | Arabidopsis thaliana chromosome II BAC F4P9 genomic sequence, complete sequence. | |
| 700346956H1 | g2737925 | 19 | 4 | gb105pln | Arabidopsis thaliana fimbrin-like protein AtFim2 mRNA, partial cds. | |
| 700343156H1 | g219929 | 8 | 7 | gb105allp | midkine | |
| 700343239H1 | g2191168 | 12 | 1 | gb105eukp | A_IG002P16.20 | |
| 700346045H1 | g1167857 | 21 | −29 | gb105pln | S. cereale cv. Petkus ‘Halo’ encoding cpn60. | |
| 700345275H1 | g602605 | 35 | −45 | gb105pln | Zea mays tandem genes for alpha-tubulin and alpha2-tubulin. | |
| 700342657H1 | g1236731 | 35 | 0 | gb105allp | prolyl aminopeptidase | |
| 700343119H1 | g882393 | 24 | 6 | gb105allp | TBP-associated factor TAFII31 | |
| 700281925H1 | g166410 | 36 | −10 | gb105eukp | nucleic acid binding protein; Alfin-1 | |
| 700343631H1 | g224829 | 27 | −8 | gb105pln | Arabidopsis thaliana DNA chromosome 4, ESSA I contig fragment No. 2. | |
| 700345072H1 | g473206 | 8 | 3 | gb105allp | mitochondrial malate dehydrogenase | |
| 700345889H1 | g2662342 | 70 | −55 | gb105pln | Oryza sativa mRNA for EF-1 alpha, complete cds. | |
| 700346710H1 | g2264311 | 12 | 15 | gb105pln | Arabidopsis thaliana genomic DNA, chromosome 5, P1 clone: MLN1, complete | |
| | | | | | sequence. | |
| 700344044H1 | g416554 | 40 | −5 | gb105allp | ribosomal protein L18a (AA 1-175) | |
| 700380653H1 | g886739 | 46 | −35 | gb105pln | Z. mays histone H4 gene. | |
| 700343156H1 | g188571 | 8 | 7 | gb105allp | retinoic acid inducible factor | |
| 700380647H1 | g508307 | 38 | −26 | gb105pln | Arabidopsis thaliana protein-serine kinase mRNA, complete cds. | |
| 700346836H1 | g575959 | 92 | −76 | gb105pln | Z. mays (Black Mexican Sweet) mRNA for 1-acyl-glycerol-3-phosphate acyltransferase | |
| | | | | | (putative). | |
| 700342453H1 | g166816 | 43 | −35 | gb105pln | Arabidopsis thaliana protein kinase mRNA, complete cds. | |
| 700343120H1 | g2246624 | 28 | −1 | gb105pln | Oryza sativa protein kinase mRNA, complete cds. | |
| 700344325H1 | g2367200 | 9 | 5 | gb105allp | tRNA pseudouridine 55 synthase (psi55 synthase) (p35 protein) | |
| 700344953H1 | g2244829 | 19 | 4 | gb105pln | Arabidopsis thaliana DNA chromosome 4, ESSA I contig fragment No. 2. | |
| 700343079H1 | g2150046 | 9 | 7 | gb105allp | 26S proteasome subunit 9 | |
| 700346465H1 | g1147800 | 44 | −25 | gb105eukp | SUG2; Sug2p | |
| 700344192H1 | g2078349 | 41 | −29 | gb105pln | Solanum tuberosum transaldolase (PotTall) mRNA, complete cds. | |
| 700345752H1 | g2088638 | 20 | −3 | gb105pln | Arabidopsis thaliana chromosome II BAC T28M21 genomic sequence, complete | |
| | | | | | sequence. | |
| 700379965H1 | g498774 | 78 | −43 | gb105pln | Z. mays (cv DH5xDH7) hsp70-5 mRNA for heat shock protein 70. | |
| 700343453H1 | g1053058 | 34 | −32 | gb105pln | Triticum aestivum histone H3 gene, partial cds, clone W2. | |
| 700342378H1 | g168494 | 52 | −34 | gb105pln | Maize (Zea mays) histone H3 gene (H3C2), complete cds. | |
| 700344747H1 | g303855 | 16 | −1 | gb105eukp | ribosomal protein L7A | |
| 700343110H1 | g602367 | 13 | 15 | gb105pln | Yeast (Saccharomyces cerevisiae) chromosome V cosmids 9871, 8199, 9867, 9495 and | |
| | | | | | lambda clones 6693 and 5898. | |
| 700344856H1 | g2244839 | 17 | −8 | gb105eukp | hypothetical protein | |
| 700347371H1 | g2529657 | 13 | 16 | gb105pln | Arabidopsis thaliana chromosome II BAC T30B22 genomic sequence, complete | |
| | | | | | sequence. | |
| 700344026H1 | g167961 | 65 | −58 | gb105pln | D. caryophyllus S-adenosylmethionine synthetase (CARSAM2) mRNA, complete cds. | |
| 700342490H1 | g168498 | 76 | −79 | gb105pln | Corn histone H4 (H4C13) gene, complete cds. | |
| 700379947H1 | g2191193 | 14 | 1 | gb105eukp | A_TM021B04.8 | |
| 700343934H1 | g473604 | 79 | −63 | gb105pln | Zea mays W-22 histone H2B mRNA, complete cds. | |
| 700345085H1 | g310314 | 75 | −68 | gb105pln | Oryza sativa calmodulin gene, complete cds. | |
| 700342734H1 | g432367 | 44 | −38 | gb105pln | Rice mRNA for elongation factor 1 beta, complete cds. | |
| 700343582H1 | g166866 | 18 | 3 | gb105pln | A. thaliana ribosomal protein S11 mRNA, 3′ end. | |
| 700347341H1 | g633599 | 27 | −6 | gb105pln | N. tabacum (SR1) aox mRNA. | |
| 700342305H1 | g1929409 | 34 | −17 | gb105pln | N. tabacum npp4 mRNA for protein phosphatase Type 2A. | |
| 700344260H1 | g416251 | 23 | −8 | gb105pln | Rice mRNA for acetohydroxy acid reductoisomerase, partial sequence. | |
| 700343104H1 | g433492 | 19 | −5 | gb105allp | HLA-3 | |
| 700343514H1 | g600709 | 16 | 2 | gb105pln | Arabidopsis thaliana synaptobrevin-related protein (SAR1) mRNA, complete cds. | |
| 700380655H1 | g1296954 | 27 | −52 | gb105pln | O. sativa mRNA for novel protein, osr40c1. | |
| 700345549H1 | g485376 | 46 | −32 | gb105pln | Zea mays alpha-3-tubulin gene, complete cds. | |
| 700281817H2 | g1049197 | 8 | 2 | gb105allp | RecA | |
| 700343725H1 | g2388937 | 37 | −9 | gb105eukp | SPAC23H4.18c; hypothetical protein | |
| 700344689H1 | g443591 | 34 | 4 | gb105eukp | rp119; bind calmodulin; ribosomal protein; v14 gene | |
| 700345008H1 | g2262141 | 18 | −5 | gb105eukp | T10P11.8; hypothetical protein | |
| 700345561H1 | g1321660 | 37 | −35 | gb105pln | Rice mRNA for ascorbate peroxidase, complete cds. | |
| 700345416H1 | g798817 | 37 | −29 | gb105pln | A. thaliana mRNA for ribosomal protein L2. | |
| 700344814H1 | g1103391 | 31 | −14 | gb105eukp | GCTK; signal transduction; Sponge Receptor tyrosine kinase; EC 2.7.1.112 | |
| 700344968H1 | g21634 | 13 | 5 | gb105pln | T. aestivum 1b-c38 gene for HBP-1b (leucine zipper type transcription factor). | |
| 700346731H1 | g2293565 | 82 | −78 | gb105pln | Oryza sativa ADP-ribosylation factor 1 (Os-ARF1) mRNA, complete cds. | |
| 700342989H1 | g2668737 | 65 | −55 | gb105pln | Zea mays translation initiation factor 5A (TIF5A) mRNA, complete cds. | |
| 700344253H1 | g1706957 | 24 | −7 | gb105pln | Gossypium hirsutum cellulose synthase (celA2) mRNA, partial cds. | |
| 700281870H2 | g1418318 | 35 | −29 | gb105pln | A. thaliana CER1-like gene (1992bp). | |
| 700379679H1 | g398607 | 26 | −21 | gb105pln | A. thaliana mRNA for elongation factor 1 beta. | |
| 700342785H1 | g18057 | 39 | 11 | gb105pln | Citrus limon 26S ribosomal RNA gene 3′ region. | |
| 700344808H1 | g2642212 | 14 | 2 | gb105pln | Zea mays nitrate-induced NOI protein gene, complete cds. | |
| 700345865H1 | g13339 | 30 | −11 | gb105pln | P. hybrida mitochondrial rps19 gene for ribosomal protein S19. | |
| 700346080H1 | g1055087 | 11 | −4 | gb105eukp | W06B11.2 | |
| 700346108H1 | g945086 | 30 | −12 | gb105pln | Glycine max transcription factor TFIIB mRNA, complete cds. | |
| 700343083H1 | g1212995 | 21 | −23 | gb105pln | H. vulgare mRNA for UDP-glucose pyrophosphorylase. | |
| 700380456H1 | g1881692 | 41 | −68 | gb105pln | Zea mays phosphoglucomutase mRNA, partial cds. | |
| 700342573H1 | g1514579 | 54 | −37 | gb105allp | immunoglobulin kappa light chain | |
| 700345093H1 | g1929405 | 57 | −48 | gb105pln | N. tabacum npp2 mRNA for protein phosphatase type 1. | |
| 700346255H1 | g2702261 | 16 | 6 | gb105pln | Arabidopsis thaliana chromosome II BAC T21L14 genomic sequence, complete | |
| | | | | | sequence. | |
| 700346917H1 | g486060 | 12 | 3 | gb105eukp | PRI2 | |
| 700343138H1 | g1072389 | 16 | 0 | gb105pln | N. tabacum mRNA for cyclin A-like protein (clone 13). | |
| 700345595H1 | g1747418 | 89 | −1 | gb105pln | Z. mays mRNA for eukaryotic initiation factor-5. | |
| 700344691H1 | g434344 | 29 | −14 | gb105pln | A. thaliana (Columbia) mRNA for S18 ribosomal protein (641bp). | |
| 700346762H1 | g1881694 | 86 | −77 | gb105pln | Zea mays phosphoglucomutase mRNA, partial cds. | |
| 700342229H1 | g998429 | 47 | −60 | gb105pln | GRF1 = general regulatory factor [Zea mays, XL80, Genomic, 5348 nt]. | |
| 700346927H1 | g459009 | 27 | −2 | gb105eukp | T20H4.3 | |
| 700343258H1 | g1944132 | 8 | 5 | gb105allp | CUC2 | |
| 700343418H1 | g310932 | 38 | −27 | gb105pln | Nicotiana tabacum ribosomal protein L17 mRNA, complete cds. | |
| 700380763H1 | g497119 | 31 | −23 | gb105pln | Pisum sativum monodehydroascorbate reductase mRNA, complete cds. | |
| 700344292H1 | g404165 | 33 | −14 | gb105pln | A. thaliana gene for BBC1 protein. | |
| 700380794H1 | g166409 | 30 | −18 | gb105pln | Alfalfa nucleic acid binding protein (alfin-1) mRNA, partial cds. | |
| 700346529H1 | g1129085 | 28 | −14 | gb105pln | Wheat mRNA for protein H2A, complete cds, clone wcH2A-9. | |
| 700344738H1 | g2459442 | 22 | −3 | gb104eukp | F4P9.39; putative DNA-binding protein PD1 | |
| 700281909H2 | g166410 | 9 | 3 | gb105eukp | nucleic acid binding protein; Alfin-1 | |
| 700343331H1 | g1402878 | 15 | 1 | gb105eukp | orf4; unknown | |
| 700347171H1 | g2104889 | 16 | 5 | gb105eukp | YOR29-28 | |
| 700347125H1 | g2317873 | 45 | −38 | gb105pln | Prunus armeniaca Rab7 GTP binding protein mRNA, complete cds. | |
| 700345736H1 | g166859 | 35 | −38 | gb105pln | Arabidopsis thaliana ribosomal protein gene, complete cds. | |
| 700345480H1 | g2182285 | 14 | 6 | gb105pln | Sequence of bAC F5I14 from Arabidopsis thaliana chromosome 1, complete sequence. | |
| 700345768H1 | g2463568 | 19 | 6 | gb105pln | Glycine max mRNA for squalene synthase, complete cds. | |
| 700343892H1 | g168500 | 54 | −42 | gb105pln | Maize (Zea mays) histone H4 gene (H4C14), complete cds. | |
| 700380786H1 | g310934 | 12 | 17 | gb105pln | Nicotiana tabacum ribosomal protein L25, complete cds. | |
| 700346378H1 | g642243 | 22 | −11 | gb105pln | H. vulgare cdr29 gene. | |
| 700281804H2 | g557681 | 55 | −46 | gb105pln | Z. mays mRNA for ribosomal protein S28. | |
| 700346389H1 | g2656030 | 20 | 7 | gb105pln | Arabidopsis thaliana genomic DNA, chromosome 5, P1 clone: MUL8. | |
| 700346351H1 | g895693 | 11 | 6 | gb105eukp | ribosomal protein S15a | |
| 700347257H1 | g1519250 | 62 | −53 | gb105pln | Oryza sativa GF14-c protein mRNA, complete cds. | |
| 700346427H1 | g2749918 | 12 | 15 | gb105pln | Arabidopsis thaliana chromosome I BAC F3I6 genomic sequence, complete sequence. | |
| 700345145H1 | g2696018 | 22 | −1 | gb105pln | Arabidopsis thaliana genomic DNA, chromosome 5, P1 clone: MXC9. | |
| 700380212H1 | g170746 | 30 | −38 | gb105pln | Wheat histone H4 TH091 gene, complete cds. | |
| 700346872H1 | g940536 | 15 | 3 | gb105allp | P1 Cdc21 protein | |
| 700342557H1 | g587545 | 24 | −31 | gb105pln | S. tuberosum mRNA encoding homolog to Human P23 tumor protein. | |
| 700342262H1 | g1498053 | 13 | −2 | gb105eukp | ribosomal protein S8 | |
| 700380256H1 | g1360017 | 16 | 2 | gb105allp | U1 snRNP-specific protein C | |
| 700345918H1 | g1885343 | 42 | −59 | gb105pln | T. aestivum mRNA for starch branching enzyme II. | |
| 700346688H1 | g2564336 | 44 | −30 | gb105pln | Brassica campestris mRNA for Tat binding protein 1, complete cds. | |
| 700346120H1 | g1946624 | 12 | −4 | gb105eukp | COII; cytochrome c oxidase subunit II | |
| 700344090H1 | g2632251 | 86 | −78 | gb105pln | S. bicolor DNA for gene encoding putative protein serine/threonine kinase, clone | |
| | | | | | cSNFL1. | |
| 700345911H1 | g436031 | 28 | −15 | gb105pln | Nicotiana tabacum (TSC40-4) 60S ribosomal protein L34 mRNA, complete cds. | |
| 700345490H1 | g409131 | 18 | −14 | gb105eukp | mei-1; meiotic spindle formation | |
| 700380786H1 | g2654121 | 12 | 15 | gb105pln | Arabidopsis thaliana ribosomal protein L23a (AtrpL23a) mRNA, complete cds. | |
| 700347055H1 | g1905943 | 37 | −21 | gb105pln | Sorghum bicolor MADS box transcription factor SbMADS1 mRNA, complete cds. | |
| 700344506H1 | g2460124 | 40 | −9 | gb105allp | putative transcription factor CA150 | |
| 700347289H1 | g1401051 | 4 | 8 | gb105allp | similar to Dvl-1 product encoded by GenBank Accession Number U10115; dishevelled | |
| | | | | | segment polarity protein homolog | |
| 700380496H1 | g551450 | 10 | 7 | gb105allp | splicing factor SF3a60 | |
| 700342808H1 | g603220 | 32 | −18 | gb105pln | Medicago sativa 6-phosphogluconate dehydrogenase mRNA, complete cds. | |
| 700343334H1 | g1418331 | 17 | −4 | gb105eukp | LRK1; receptor like protein kinase | |
| 700343511H1 | g1439625 | 9 | 7 | gb105eukp | C52B9.4 | |
| 700345077H1 | g603569 | 27 | −1 | gb105pln | L. esculentum mRNA for ribosomal protein S17. | |
| 700342488H1 | g450292 | 43 | −18 | gb105pln | Z. mays alpha-tubulin mRNA, complete cds. | |
| 700379690H1 | g2317728 | 25 | −0 | gb105pln | Arabidopsis thaliana reversibly glycosylated polypeptide-1 (AtRGP) mRNA, | |
| | | | | | complete cds. | |
| 700344504H1 | g297877 | 23 | −0 | gb105pln | A. thaliana UBC10 mRNA for ubiquitin conjugating enzyme homolog. | |
| 700345638H1 | g1869859 | 5 | 7 | gb105allp | very large tegument protein | |
| 700281841H2 | g602605 | 56 | −79 | gb105pln | Zea mays tandem genes for alpha1-tubulin and alpha2-tubulin. | |
| 700347234H1 | g2459424 | 16 | 0 | gb105eukp | F4P9.19 | |
| 700347166H1 | g313151 | 20 | 2 | gb105pln | A. thaliana ribosomal protein S15 mRNA, complete CDS. | |
| 700346739H1 | g510337 | 9 | −3 | gb105eukp | lipid transfer protein | |
| 700342555H1 | g386765 | 11 | −19 | gb105allp | hba1 alpha globin | |
| 700346922H1 | g2384908 | 36 | −7 | gb105eukp | W03F9.10 | |
| 700343447H1 | g899609 | 61 | −43 | gb105pln | Zea mays acidic ribosomal protein P2 (RPA-2A1) mRNA, complete cds. | |
| 700343144H1 | g168423 | 87 | −54 | gb105pln | Zea mays polypeptide chain-binding protein mRNA, 3′ end. | |
| 700345410H1 | g2191149 | 51 | −26 | gb105eukp | Ap_IG002N01.22 | |
| 700347382H1 | g644492 | 96 | −82 | gb105pln | Corn elongation factor 1alpha gene, complete cds. | |
| 700342551H1 | g1710150 | 18 | 2 | gb105pln | Arabidopsis thaliana proline iminopeptidase mRNA, complete cds. | |
| 700342237H1 | g531548 | 6 | 5 | gb105eukp | PRD1; saccharolysin | |
| 700343901H1 | g1054795 | 68 | −65 | gb105pln | H. vulgare mRNA for transmembrane protein. | |
| 700342401H1 | g2190545 | 18 | −1 | gb105eukp | F5I14.8 | |
| 700343331H1 | g2252840 | 14 | 1 | gb105allp | contains regions of similarity to Haemophilus influenzae permease (SP:P38767) | |
| 700342446H1 | g1049252 | 84 | −70 | gb105pln | Zea mays vacuolar ATPase 69 kDa subunit mRNA, partial cds. | |
| 700343570H1 | g790473 | 48 | −11 | gb105eukp | soluble, glycine rich protein | |
| 700346272H1 | g1550738 | 13 | 3 | gb105allp | endomembrane-associated protein | |
| 700344134H1 | g2196671 | 80 | −26 | gb105pln | Z. mays mRNA for HMG protein. | |
| 700346867H1 | g2114208 | 63 | −70 | gb105pln | Wheat DNA for 18S rRNA and telomere repeat sequence. | |
| 700345577H1 | g2645165 | 58 | 0 | gb105pln | Oryza sativa mRNA, similar to ribosomal protein 41. | |
| 700344130H1 | g457708 | 24 | −20 | gb105pln | S. oleracea mRNA for protein kinase. | |
| 700344859H1 | g950199 | 13 | 5 | gb105allp | mitotic feedback control protein Madp2 homolog | |
| 700379614H1 | g22151 | 94 | −48 | gb105pln | Z. mays (A188) mRNA for alpha-tubulin 4. | |
| 700343057H1 | g2618721 | 33 | −12 | gb105eukp | IAA16; IAA16 | |
| 700344630H1 | g2352492 | 7 | 5 | gb105eukp | TIR1; transport inhibitor response 1 | |
| 700344203H1 | g1695692 | 18 | 5 | gb105eukp | diminuto | |
| 700345362H1 | g2739218 | 44 | −22 | gb105pln | Hordeum vulgare S28 ribosomal protein. | |
| 700379734H1 | g2388574 | 10 | 6 | gb105allp | Strong similarity to Phalaenopsis homeobox protein (gb\|U34743). | |
| 700345250H1 | g473604 | 29 | −13 | gb105pln | Zea mays W-22 histone H2B mRNA, complete cds. | |
| 700346295H1 | g1834423 | 15 | −3 | gb105eukp | ribosomal protein L14 | |
| 700344323H1 | g577824 | 21 | 1 | gb105pln | Z. mays gene for H2B histone (gH2B3). | |
| 700344667H1 | g38223 | 75 | 3 | gb105allp | alpha1-globin | |
| 700346908H1 | g168460 | 89 | −72 | gb105pln | Zea mays cyclophilin (CyP) mRNA, complete cds. | |
| 700343962H1 | g218180 | 63 | −58 | gb105pln | Rice mRNA for oryzain alpha (EC 3.4.22). | |
| 700346981H1 | g218160 | 24 | −15 | gb105pln | Oryza sativa mRNA for elongation factor 1 beta′. | |
| 700346885H1 | g2511578 | 41 | −11 | gb105eukp | prcfa; multicatalytic endopeptidase; EC 3.4.99.46 | |
| 700343411H1 | g1915974 | 17 | −2 | gb105eukp | FK; fructokinase; EC 2.7.1.4 | |
| 700343183H1 | g736271 | 54 | −54 | gb105pln | O. sativa hsp70 gene for heat shock protein 70. | |
| 700347384H1 | g397395 | 59 | −39 | gb105pln | Z. mays MNB1b mRNA for DNA-binding protein. | |
| 700346602H1 | g536895 | 65 | −57 | gb105pln | Wheat mRNA for protein H2A, complete cds, clone wcH2A-10. | |
| 700344163H1 | g456567 | 34 | −16 | gb105pln | Pisum sativum ubiquitin conjugating enzyme (UBC4), complete cds. | |
| 700346380H1 | g532571 | 14 | 10 | gb105pln | Barley lipoxygenase 1 (LoxA) gene, complete cds. | |
| 700343828H1 | g1754994 | 67 | −52 | gb105pln | Tritiucum aestivum calmodulin TaCaM1-3 mRNA, complete cds. | |
| 700347058H1 | g22324 | 43 | −51 | gb105pln | Z. mays mRNA for H2B histone (clone cH2B221). | |
| 700281805H2 | g1906827 | 50 | −42 | gb105pln | A. thaliana hsp81.4 gene. | |
| 700344009H1 | g510697 | 10 | 8 | gb105eukp | pol; protease, reverse transcriptase, RNaseH, integrase; pol polyprotein | |
| 700342715H1 | g2623295 | 29 | −13 | gb105eukp | T20B5.1 | |
| 700345418H1 | g473602 | 73 | −66 | gb105pln | Zea mays W-22 histone H2A mRNA, complete cds. | |
| 700345443H1 | g168510 | 17 | 10 | gb105pln | Z. mays protein cdc2 kinase mRNA, complete cds. | |
| 700342928H1 | g1129084 | 46 | −26 | gb105pln | Wheat mRNA for protein H2A, complete cds, clone wcH2A-3. | |
| 700344747H1 | g806545 | 23 | −2 | gb105eukp | RpL7a; ribosomal protein L7a | |
| 700346845H1 | g1165125 | 22 | 0 | gb105allp | mSUG1 protein | |
| 700380665H1 | g868002 | 27 | −23 | gb105pln | Pumpkin mRNA for aconitase, complete cds. | |
| 700346533H1 | g2282583 | 54 | −77 | gb105pln | Zea mays elongation factor 1-alpha (EF1-A) mRNA, complete cds. | |
| 700343032H1 | g2274990 | 53 | −57 | gb105pln | Hordeum vulgare mRNA for expressed sequence tag. | |
| 700344443H1 | g16087 | 16 | 7 | gb105eukp | dnaJ; DNA J protein | |
| 700345337H1 | g18903 | 21 | 1 | gb105pln | Barley mRNA for aspartic proteinase. | |
| 700346549H1 | g1272628 | 15 | 5 | gb105allp | F44G4.1 | |
| 700345885H1 | g310932 | 47 | −34 | gb105pln | Nicotiana tabacum ribosomal protein L17 mRNA, complete cds. | |
| 700342365H1 | g1667268 | 21 | −8 | gb105eukp | C01G10.9 | |
| 700345118H1 | g434342 | 24 | −6 | gb105pln | A. thaliana (C24) mRNA for S18 ribosomal protein. | |
| 700346256H1 | g54006 | 20 | −3 | gb105allp | ribosomal protein S12 (AA 1-132) | |
| 700346381H1 | g1707641 | 46 | −43 | gb105pln | O. sativa mRNA for leucine rich repeat receptor-like kinase. | |
| 700345248H1 | g2746335 | 13 | 4 | gb105allp | RING zinc finger protein | |
| 700379968H1 | g2696803 | 40 | −17 | gb105pln | Oryza sativa mRNA for water channel protein, complete cds. | |
| 700281810H2 | g2625015 | 25 | −0 | gb105eukp | rpc10+; RNA polymerases I, II and III subunit Rpc10 | |
| 700342963H1 | g2369713 | 59 | −51 | gb105pln | Beta vulgaris cDNA for elongation factor 2. | |
| 700346956H1 | g1575594 | 68 | −62 | gb105pln | Triticum aestivum fimbrin/plastin-like mRNA, partial cds. | |
| 700281928H2 | g4392 | 19 | −5 | gb105eukp | RPL37A; ribosomal protein L37a | |
| 700345425H1 | g168405 | 84 | −25 | gb105pln | maize alcohol dehydrogenase (adh1) mrna 3′ end and flank. | |
| 700347034H1 | g20818 | 37 | −26 | gb105pln | P. sativum mRNA for p34 kinase-related protein (cdc2 homologue). | |
| 700346422H1 | g1325967 | 21 | −0 | gb105pln | T. aestivum histone H2A gene (clone TH274). | |
| 700342648H1 | g1054632 | 39 | −27 | gb105pln | A. thaliana mRNA for ATN1 protein kinase. | |
| 700345561H1 | g2274983 | 33 | −32 | gb105pln | Hordeum vulgare mRNA for expressed sequence tag. | |
| 700343712H1 | g669022 | 19 | 1 | gb105eukp | W06E11.4 | |
| 700343841H1 | g2394230 | 14 | 15 | gb105pln | Arabidopsis thaliana WD-40 repeat protein (MSI2) mRNA, complete cds. | |
| 700344396H1 | g1550813 | 70 | −68 | gb105pln | Z. mays mRNA for acidic ribosomal protein P0. | |
| 700344990H1 | g2653289 | 19 | 1 | gb105pln | Petunia hybrida pte gene. | |
| 700344604H1 | g1743008 | 33 | −24 | gb105pln | C. sativus mRNA for SNF1-related protein kinase. | |
| 700347375H1 | g21634 | 13 | 5 | gb105pln | T. aestivum 1b-c38 gene for HBP-1b (leucine zipper type transcription factor). | |
| 700343163H1 | g1183866 | 9 | −3 | gb105eukp | SBP1; squamosa-promoter binding protein 1 | |
| 700343961H1 | g785067 | 12 | 5 | gb105eukp | VPS27; vacuolar protein sorting; Vps27p | |
| 700347282H1 | g169295 | 27 | −17 | gb105pln | Pharbitis nil heat shock protein 83 (Hsp83) gene, complete cds. | |
| 700345974H1 | g736271 | 65 | −14 | gb105pln | O. sativa hsp70 gene for heat shock protein 70. | |
| 700344094H1 | g4352 | 14 | 6 | gb105pln | Yeast (Saccharomyces cerevisiae) gene for RNA-helicase. | |
| 700380449H1 | g1737490 | 9 | 6 | gb105allp | p76 | |
| 700379726H1 | g395071 | 25 | −4 | gb105pln | V. faba guanine nucleotide regulatory protein mRNA, complete CDS. | |
| 700347358H1 | g1185553 | 38 | −81 | gb105pln | Zea mays glyceraldehyde-3-phosphate dehydrogenase (gpc2) gene, complete cds. | |
| 700343005H1 | g829147 | 94 | −79 | gb105pln | Z. mays gene for cyclophilin. | |
| 700342334H1 | g450292 | 86 | −72 | gb105pln | Zea mays alpha-tubulin mRNA, complete cds. | |
| 700380217H1 | g169128 | 17 | 4 | gb105eukp | regulatory subunit of an atp-dependent protease; nuclear encoded precursor to chloroplast | |
| | | | | | protein | |
| 700347261H1 | g902583 | 45 | −70 | gb105pln | Zea mays clone MubG1 ubiquitin gene, complete cds. | |
| 700281830H2 | g2827138 | 19 | 9 | gb105pln | Arabidopsis thaliana cellulose synthase catalytic subunit (RSW1) gene, complete cds. | |
| 700344093H1 | g169820 | 52 | −44 | gb105pln | Oryza sativa triosephosphate isomerase (Rictpi) mRNA, complete cds. | |
| 700342361H1 | g1488296 | 27 | −18 | gb105pln | Oryza sativa osRAD23 mRNA, complete cds. | |
| 700380667H1 | g416148 | 85 | −31 | gb105pln | Zea mays beta-7 tubulin (tub7) mRNA, complete cds. | |
| 700342824H1 | g2511530 | 57 | −47 | gb105pln | Eleusine indica alpha tubulin 1 (TUA1) mRNA, complete cds. | |
| 700342606H1 | g1136436 | 24 | 0 | gb105allp | KIAA0188 | |
| 700380143H1 | g1050540 | 16 | −8 | gb105eukp | tRNA-glutamine synthetase | |
| 700342512H1 | g454356 | 37 | −27 | gb105pln | Arabidopsis thaliana Columbia glutamate-1-semialdehyde-2,1-aminomutase (gsa1) gene. | |
| 700347351H1 | g2286150 | 49 | −70 | gb105pln | Zea mays translation initiation factor (eIF-4A) mRNA, complete cds. | |
| 700342756H1 | g1145694 | 26 | −3 | gb105pln | Arabidopsis thaliana actin (ACT3) gene, complete cds. | |
| 700343034H1 | g558648 | 10 | 6 | gb105eukp | D-myo-inositol-3-phosphate synthase; EC 5.5.1.4 | |
| 700343947H1 | g2739431 | 10 | 3 | gb105allp | hematopoietic-specific IL-2 deubiquitinating enzyme | |
| 700345767H1 | g2760836 | 13 | 4 | gb105allp | putative Ser/Thr protein kinase | |
| 700344026H1 | g450548 | 70 | −62 | gb105pln | O. sativa (pRSAM-1) gene for S-adenosyl methionine synthetase. | |
| 700344212H1 | g3075 | 20 | 5 | gb105pln | N. crassa mRNA for ribosomal protein. | |
| 700380653H1 | g170746 | 43 | −32 | gb105pln | Wheat histone H4 TH091 gene, complete cds. | |
| 700342357H1 | g347061 | 34 | −12 | gb105pln | Brassica rapa ribosomal protein L37A (RPL37A) mRNA, complete cds. | |
| 700342364H1 | g1546918 | 51 | −70 | gb105pln | Z. mays mRNA for translation initiation factor 5A. | |
| 700342955H1 | g2331300 | 47 | −72 | gb105pln | Zea mays ribosomal protein S4 type I (rps4) mRNA, complete cds. | |
| 700344241H1 | g416150 | 76 | −74 | gb105pln | Zea mays beta-8 tubulin (tub8) mRNA, complete cds. | |
| 700344337H1 | g1730332 | 19 | −5 | gb105eukp | (R)-(+)-mandelonitrile lyase isoform MDL1 precursor | |
| 700380130H1 | g1628440 | 12 | 4 | gb105eukp | ORF | |
| 700345964H1 | g2564046 | 25 | −2 | gb105pln | Arabidopsis thaliana genomic DNA, chromosome 5, P1 clone: MGI19, complete | |
| | | | | | sequence. | |
| 700343056H1 | g18918 | 8 | −6 | gb105eukp | beta-amylase (AA 1-535) | |
| 700380647H1 | g1526412 | 41 | −31 | gb105pln | Arabidopsis thaliana ATPK19 mRNA for ribosomal-protein S6 kinase homolog, | |
| | | | | | complete cds. | |
| 700342476H1 | g21800 | 31 | −32 | gb105pln | T. aestivum L mRNA for histone H2B. | |
| 700343417H1 | g1053093 | 29 | −2 | gb105eukp | zds; zeta-carotene desaturase precursor | |
| 700345023H1 | g2641235 | 18 | −2 | gb105eukp | ACT1; actin | |
| 700344340H1 | g2511592 | 31 | −0 | gb105eukp | prc8; multicatalytic endopeptidase complex, proteasome component, alpha subunit; | |
| | | | | | EC 3.4.99.46 | |
| 700343582H1 | g474006 | 18 | −6 | gb105pln | Rice mRNA, partial homologus to ribosomal protein S11 gene. | |
| 700343146H1 | g2191157 | 22 | −8 | gb105pln | Arabidopsis thaliana BAC IG002P16. | |
| 700380108H1 | g1432082 | 23 | −3 | gb105pln | Arabidopsis thaliana Skp1p homolog mRNA, complete cds. | |
| 700345862H1 | g2275202 | 39 | −20 | gb105eukp | T08I13.8; acyl-CoA synthetase isolog | |
| 700342760H1 | g287834 | 51 | −34 | gb105pln | Z. mays yptm2 cDNA. | |
| 700343011H1 | g2262105 | 25 | −7 | gb105eukp | T19F06.8 | |
| 700281839H2 | g2791947 | 18 | 12 | gb105pln | Lupinus luteus mRNA for ribosomal protein L13a. | |
| 700344130H1 | g457688 | 21 | −1 | gb105pln | M. crystallinum mRNA for protein kinase. | |
| 700344144H1 | g1929411 | 27 | −6 | gb105pln | N. tabacum npp5 mRNA for protein phosphatase type 2A. | |
| 700345625H1 | g1666176 | 52 | −39 | gb105pln | N. tabacum mRNA for subunit of NADH:ubiquinone oxidoreductase complex I. | |
| 700343677H1 | g21696 | 28 | −7 | gb105pln | T. aestivum mRNA 3 for cathepsin B (2557). | |
| 700379630H1 | g2351064 | 12 | 14 | gb105pln | Arabidopsis thaliana genomic DNA, chromosome 5, P1 clone: MDJ22. | |
| 700346880H1 | g143187) | 15 | 2 | gb105allp | ent-kaurene synthase B | |
| 700345914H1 | g2642212 | 32 | −11 | gb105pln | Zea mays nitrate-induced NOI protein gene, complete cds. | |
| 700281947H2 | g22151 | 82 | −48 | gb105pln | Z. mays (A188) mRNA for alpha-tubulin 4. | |
| 700281831H2 | g2760166 | 24 | −3 | gb105pln | Arabidopsis thaliana genomic DNA, chromosome 5, P1 clone: MBK20, complete | |
| | | | | | sequence. | |
| 700347216H1 | g2244837 | 28 | −5 | gb105eukp | proteasome chain protein | |
| 700345537H1 | g995562 | 17 | −2 | gb105eukp | ada1; AMP deaminase | |
| 700343515H1 | g2384757 | 56 | −44 | gb105pln | Oryza sativa GDP dissociation inhibitor protein OsGDI1 (OsGDI1) mRNA, | |
| | | | | | complete cds. | |
| 700344482H1 | g2766715 | 57 | −7 | gb105eukp | CMT1; chromomethylase | |
| 700347003H1 | g2738948 | 21 | −14 | gb105pln | Fragaria x ananassa cytosolic ascorbate peroxidase (APX-c) mRNA, complete cds. | |
| 700345327H1 | g2564049 | 14 | 9 | gb105pln | Arabidopsis thaliana genomic DNA, chromosome 5, P1 clone: MLE2, complete | |
| | | | | | sequence. | |
| 700379931H1 | g2724049 | 27 | −12 | gb105pln | Fritillaria agrestis clone CA1 glutaredoxin type I (glr1) mRNA, complete cds. | |
| 700346972H1 | g1419369 | 76 | −68 | gb105pln | Z. mays ZmABP3 mRNA for actin depolymerizing factor. | |
| 700346623H1 | g2293565 | 35 | −14 | gb105pln | Oryza sativa ADP-ribosylation factor 1 (Os-ARF1) mRNA, complete cds. | |
| 700342663H1 | g1008274 | 10 | 0 | gb105eukp | ORF YJL097w | |
| 700343641H1 | g396148 | 12 | 8 | gb105pln | Z. mays CHI gene. | |
| 700344177H1 | g2827715 | 9 | 3 | gb105allp | receptor protein kinase-like protein | |
| 700346342H1 | g216 | 13 | 4 | gb105allp | endonexin | |
| 700346064H1 | g2114363 | 10 | 5 | gb105allp | similar to mouse Int-6 | |
| 700343964H1 | g303834 | 14 | 14 | gb105pln | Rice mRNA for 21 kd polypeptide, complete cds. | |
| 700347270H1 | g577734 | 42 | −30 | gb105pln | A. thaliana PRL1 mRNA. | |
| 700345885H1 | g2341023 | 44 | −30 | gb105pln | Sequence of BAC F19P19 from Arabidopsis thaliana chromosome 1, complete | |
| | | | | | sequence. | |
| 700345545H1 | g1181672 | 51 | −48 | gb105pln | Sorghum bicolor heat shock protein 70 cognate (hsc70) mRNA, partial cds. | |
| 700342648H1 | g2656028 | 15 | 15 | gb105pln | Arabidopsis thaliana genomic DNA, chromosome 5, P1 clone: MNF13. | |
| 700342570H1 | g791101 | 19 | 0 | gb105pln | Yeast (Saccharomyces cerevisiae) PMS1, TPM1, MKS1, YMK1, MSK1, ODP2, YL9A | |
| | | | | | & FKH2 genes. | |
| 700346686H1 | g20163 | 42 | −23 | gb105pln | O. sativa Rr15 mRNA for 5S ribosomal RNA. | |
| 700342705H1 | g2624327 | 38 | −36 | gb105pln | Oryza sativa mRNA for glycine rich RNA-binding protein 2 (OsGRP2). | |
| 700343716H1 | g533251 | 69 | −70 | gb105pln | Zea mays (clone pSM8) sucrose synthase 2 (Sus1) gene, complete cds. | |
| 700344120H1 | g1914683 | 21 | 8 | gb105allp | RAD23, isoform I | |
| 700345133H1 | g432367 | 42 | −32 | gb105pln | Rice mRNA for elongation factor 1 beta, complete cds. | |
| 700347050H1 | g1553130 | 15 | 16 | gb105pln | Gossypium hirsutum ribosomal protein L44 isoform b (RL44), complete cds. | |
| 700281804H2 | g2264307 | 14 | −4 | gb105pln | Arabidopsis thaliana genomic DNA, chromosome 5, P1 clone: MED24, complete | |
| | | | | | sequence. | |
| 700380481H1 | g2623158 | 14 | 2 | gb105allp | N-acetylglucosamyl transferase component Gpi1 | |
| 700342372H1 | g168505 | 62 | −56 | gb105pln | Zea mays histone H3 gene, complete cds. | |
| 700343204H1 | g435679 | 19 | 2 | gb105eukp | ribosomal protein S25 | |
| 700344346H1 | g2342425 | 10 | 8 | gb105eukp | ANP2; NPK1-related protein kinase 2 | |
| 700344445H1 | g1737072 | 15 | −5 | gb105allp | periodic tryptophan protein 2 | |
| 700347208H1 | g172578 | 10 | 7 | gb105eukp | SEN3; affects tRNA processing | |
| 700344150H1 | g2541973 | 51 | −1 | gb105allp | DNA damage-inducible RNA binding protein | |
| 700347285H1 | g17601 | 13 | 3 | gb105allp | AUXIN REGULATED PROTEIN | |
| 700347282H1 | g20255 | 20 | −27 | gb105pln | O. sativa gene for heat shock protein 82 HSP82. | |
| 700342916H1 | g22502 | 58 | −83 | gb105pln | Zea mays DNA fragment for deletion derivative Spm-I8 of En-I transposable element. | |
| 700344496H1 | g559395 | 19 | −2 | gb105eukp | dihydrolipoamide acetyltransferase (E2) subunit of PDC | |
| 700380696H1 | g433608 | 57 | −49 | gb105pln | R. communis mRNA for enolase. | |
| 700347222H1 | g170107 | 16 | 6 | gb105eukp | CPN10; chaperonin 10 | |
| 700343657H1 | g295749 | 11 | 7 | gb105eukp | His2AvD; histone H2A | |
| 700380165H1 | g1001725 | 10 | 4 | gb105allp | delta(24)-sterol C-methyltransferase | |
| 700346857H1 | g1184595 | 12 | 2 | gb105eukp | T11G6.2 | |
| 700344066H1 | g2225941 | 25 | 8 | gb105allp | Mdes protein | |
| 700281992H2 | g2570508 | 18 | −20 | gb105pln | Oryza sativa transmembrane protein mRNA, complete cds. | |
| 700343344H1 | g1783180 | 65 | −50 | gb105pln | Wheat mRNA for Thiol-specific antioxidant protein, partial cds. | |
| 700379667H1 | g2760173 | 19 | −15 | gb105pln | Arabidopsis thaliana genomic DNA, chromosome 5, P1 clone: MYH19, complete | |
| | | | | | sequence. | |
| 700281913H2 | g397958 | 60 | −71 | gb105pln | Z. mays mRNA for beta-amylase. | |
| 700345305H1 | g4392 | 14 | 1 | gb105eukp | RPL37A; ribosomal protein L37a | |
| 700379646H1 | g395076 | 22 | 2 | gb105pln | B. rapa ribosomal protein L37a mRNA, complete CDS. | |
| 700379664H1 | g22469 | 75 | −73 | gb105pln | Maize mRNA for cytoplasmic ribosomal protein S11. | |
| 700346932H1 | g168500 | 53 | −50 | gb105pln | Maize (Zea mays) histone H4 gene (H4C14), complete cds. | |
| 700345918H1 | g2340107 | 58 | −79 | gb105pln | Zea mays starch branching enzyme IIa (Sbe2a) mRNA, partial cds. | |
| 700345123H1 | g1183936 | 13 | 13 | gb105pln | P. sativum 5S rRNA gene. | |
| 700347326H1 | g533473 | 35 | −20 | gb105pln | Mesembryanthemum crystallinum 2-phospho-D-glycerate hydrolase, enolase, mRNA, | |
| | | | | | complete cds. | |
| 700379973H1 | g2209343 | 14 | 6 | gb105allp | copper transport protein Atox1 | |
| 700344064H1 | g2293565 | 79 | −79 | gb105pln | Oryza sativa ADP-ribosylation factor 1 (Os-ARF1) mRNA, complete cds. | |
| 700346011H1 | g2632280 | 17 | 1 | gb105allp | seryl-tRNA synthetase | |
| 700342277H1 | g22324 | 48 | −32 | gb105pln | Z. mays mRNA for H2B histone (clone cH2B221). | |
| 700379915H1 | g556557 | 66 | −59 | gb105pln | Rice mRNA for homlogue of Tat binding protein, complete cds. | |
| 700346829H1 | g881635 | 47 | −20 | gb105eukp | Arp3 | |
| 700345614H1 | g19103 | 41 | −25 | gb105pln | H. vulgare mRNA for ribosomal protein L17-2. | |
| 700342546H1 | g969094 | 21 | −11 | gb105eukp | chaperonin containing T-complex protein gamma subunit-like protein | |
| 700344110H1 | g166843 | 26 | −8 | gb105pln | Arabidopsis thaliana ribonucleoprotein mRNA, complete cds. | |
| 700346541H1 | g1435156 | 28 | −13 | gb105pln | L. esculentum mRNA for histone H3 variant H3.3. | |
| 700343238H1 | g2274990 | 42 | −4 | gb105pln | Hordeum vulgare mRNA for expressed sequence tag. | |
| 700345055H1 | g166548 | 68 | −46 | gb105pln | Avena sativa vacuolar H+-ATPase 16 kDa proteolipid subunit (vatp-P1) mRNA, | |
| | | | | | complete cds. | |
| 700345212H1 | g2331130 | 29 | −13 | gb105pln | Oryza sativa glycine-rich protein (OSGRP1) mRNA, complete cds. | |
| 700346096H1 | g576632 | 46 | 7 | gb105pln | Chlamydomonas reinhardtii histone H3 (ch3-I) and histone H4 (ch4-I) genes, | |
| | | | | | complete cds. | |
| 700343841H1 | g2394226 | 21 | 2 | gb105pln | Lycopersicon esculentum WD-40 repeat protein (LeMSI1) mRNA, complete cds. | |
| 700345264H1 | g2258073 | 32 | −16 | gb105pln | Hordeum vulgare var. distichum soluble inorganic pyrophosphatase (Ipp) mRNA, | |
| | | | | | complete cds. | |
| 700344038H1 | g415410 | 63 | 0 | gb105allp | 60S ribosomal protein L32 | |
| 700380440H1 | g1098619 | 19 | 4 | gb105allp | voltage dependent anion channel 2 | |
| 700380294H1 | g2739365 | 38 | −17 | gb105eukp | T9J22.7 | |
| 700380043H1 | g1022800 | 18 | −3 | gb105pln | Arabidopsis thaliana OBP33pep mRNA, partial cds. | |
| 700344734H1 | g18210 | 17 | 3 | gb105pln | C. reinhardtii mRNA for ribosomal protein P1. | |
| 700345732H1 | g565064 | 21 | −1 | gb105pln | Arabidopsis thaliana pyruvate dehydrogenase E1 beta subunit mRNA, complete cds. | |
| 700342270H1 | g2827081 | 29 | −13 | gb105pln | Medicago sativa cytosolic malate dehydrogenase (cmdh) mRNA, complete cds. | |
| 700343903H1 | g1154953 | 29 | −37 | gb105pln | T. aestivum histone H2A gene. | |
| 700379674H1 | g2318117 | 21 | 5 | gb105eukp | ChlD; one of three subunit of a complex which catalyzes the Mg insertion step in | |
| | | | | | chlorophyll biosynthesis; Mg-chelatase subunit D | |
| 700342668H1 | g19103 | 46 | −34 | gb105pln | H. vulgare mRNA for ribosomal protein L17-2. | |
| 700281923H2 | g22324 | 32 | −39 | gb105pln | Z. mays mRNA for H2B histone (clone cH2B221). | |
| 700344248H1 | g1405356 | 12 | −2 | gb105eukp | PDI; catalyses native disulphide bond formation; protein disulphide isomerase | |
| | | | | | precursor; EC 5.3.4.1 | |
| 700346571H1 | g2654010 | 15 | −9 | gb105pln | Arabidopsis thaliana Rho-like GTP binding protein (Rop5) gene, partial cds. | |
| 700346256H1 | g872315 | 20 | −3 | gb105allp | 40S ribosomal protein S12 | |
| 700344977H1 | g1350539 | 15 | 10 | gb105pln | Picea glauca EMB4 mRNA. | |
| 700346445H1 | g169818 | 48 | −60 | gb105pln | Rice 25S ribosomal RNA gene. | |
| 700344035H1 | g310934 | 58 | −28 | gb105pln | Nicotiana tabacum ribosomal protein L25, complete cds. | |
| 700344204H1 | g432488 | 18 | −5 | gb105pln | Wheat initiation factor 1A (eIF-1A) mRNA. | |
| 700343313H1 | g1532210 | 18 | 7 | gb105allp | cyclophilin A | |
| 700346985H1 | g21794 | 50 | −47 | gb105pln | Wheat histone H4 gene. | |
| 700343338H1 | g1617274 | 13 | 1 | gb105eukp | AMP-binding protein | |
| 700346831H1 | g668986 | 24 | −2 | gb105pln | S. tuberosum TYKY2 mRNA for NADH:ubiquinone oxidoreductase. | |
| 700342638H1 | g473216 | 38 | −30 | gb105pln | P. sativum (little marvel) HSC71.0 mRNA. | |
| 700343281H1 | g488835 | 9 | 5 | gb105allp | ribosomal protein L28 | |
| 700344486H1 | g607003 | 9 | 7 | gb105allp | beta transducin-like protein | |
| 700344208H1 | g22317 | 10 | 6 | gb105eukp | GST III (AA 1-222) | |
| 700342649H1 | g1209700 | 44 | −5 | gb105pln | Zea mays ribosomal protein L12 mRNA, complete cds. | |
| 700343701H1 | g313266 | 26 | −15 | gb105pln | T. aestivum gene for phosphoglycerate kinase. | |
| 700380552H1 | g28519 | 33 | −0 | gb105allp | L apoferritin | |
| 700343960H1 | g1515357 | 17 | 2 | gb105allp | CTP synthetase | |
| 700344965H1 | g167167 | 18 | −3 | gb105eukp | SKR6; receptor protein kinase | |
| 700345644H1 | g1103629 | 27 | −56 | gb105pln | Z. mays Fer2 gene. | |
| 700346379H1 | g1389653 | 30 | −9 | gb105pln | Tobacco mRNA for cytosolic ascorbate peroxidase, complete cds. | |
| 700379952H1 | g1044860 | 24 | −23 | gb105eukp | W02B12.10 | |
| 700281915H1 | g527680 | 29 | −5 | gb105eukp | ribosomal protein S3 | |
| 700346408H1 | g2801432 | 17 | 1 | gb105pln | Arabidopsis thaliana salt stress inducible small GTP binding protein Ran1 homolog | |
| | | | | | mRNA, complete cds. | |
| 700346945H1 | g19342 | 35 | −23 | gb105pln | L. esculentum mRNA for ribosomal protein L2. | |
| 700345987H1 | g1181330 | 25 | −37 | gb105pln | Z. mays CNX mRNA. | |
| 700345531H1 | g157564 | 10 | 2 | gb105eukp | Aats-glupro; trnasfer RNA-Glu-Pro aminoacyl synthetase | |
| 700345321H1 | g166857 | 35 | −18 | gb105pln | Arabidopsis thaliana cytoplasmic ribosomal protein mRNA, complete cds. | |
| 700380417H1 | g1321660 | 20 | −10 | gb105pln | Rice mRNA for ascorbate peroxidase, complete cds. | |
| 700347132H1 | g168559 | 77 | −18 | gb105pln | Zea mays putative elongation factor 1-alpha homolog mRNA, partial cds. | |
| 700342963H1 | g2641945 | 30 | −16 | gb105pln | Yeast (Schizosaccaromyces pombe) DNA for elongation factor 2, complete cds. | |
| 700343815H1 | g161172 | 17 | −0 | gb105eukp | elongation factor 1-gamma | |
| 700344808H1 | g2642213 | 32 | 3 | gb105eukp | nitrate-induced NOI protein | |
| 700346044H1 | g871513 | 34 | −18 | gb105pln | P. sativum mRNA for small G protein. | |
| 700344278H1 | g22484 | 53 | −52 | gb105pln | Z. mays RNA for superoxide dismutase Sod4A. | |
| 700379641H1 | g397631 | 26 | −6 | gb105eukp | cleavage of sucrose to glucose and fructose; beta-fructofuranosidase; EC 3.2.1.26; | |
| | | | | | invertase | |
| 700346579H1 | g1699370 | 28 | −8 | gb105allp | prolidase = peptidase D/imidopeptidase {EC 3.4.13.9} [mice, liver, Peptide, 493aa] | |
| 700343419H1 | g577824 | 40 | −43 | gb105pln | Z. mays gene for H2B histone (gH2B3). | |
| 700343631H1 | g498901 | 24 | −5 | gb105pln | Pisum sativum ribosomal protein L27 homolog (RPL27-3) mRNA, complete cds. | |
| 700344815H1 | g1684713 | 10 | 14 | gb105pln | N. racemosa mRNA for cytochrome P450, CYP71A5. | |
| 700344944H1 | g2190552 | 29 | −8 | gb105eukp | F5I14.21 | |
| 700347114H1 | g1906827 | 32 | −5 | gb105pln | A. thaliana hsp81.4 gene. | |
| 700345865H1 | g1877024 | 37 | −19 | gb105pln | Rice mitochondrial DNA for ribosomal protein L2, ribosomal protein S19, complete cds. | |
| 700380285H1 | g1335861 | 52 | −45 | gb105pln | Glycine max clathrin heavy chain mRNA, complete cds. | |
| 700343101H1 | g1766047 | 33 | −9 | gb105pln | Arabidopsis thaliana NAD+ dependent isocitrate dehydrogenase subunit 2 (IDH2) | |
| | | | | | mRNA, complete cds. | |
| 700347133H1 | g2058278 | 51 | 0 | gb105eukp | atran1 | |
| 700343208H1 | g2191187 | 9 | −2 | gb105eukp | A_TM021B04.9 | |
| 700345809H1 | g886739 | 12 | 14 | gb105pln | Z. mays histone H4 gene. | |
| 700342934H1 | g2832358 | 25 | −6 | gb105pln | Arabidopsis thaliana mRNA for HMG betal protein. | |
| 700346347H1 | g2414625 | 17 | −15 | gb105eukp | SPAC2C4.14c; hypothetical protein | |
| 700345605H1 | g1397320 | 24 | −3 | gb105eukp | R08C7.3 | |
| 700346566H1 | g984677 | 23 | −3 | gb105pln | P. sativum mRNA for lipoamide dehydrogenase. | |
| 700281856H2 | g1143863 | 33 | −17 | gb105pln | Oryza sativa beta-glucosidase mRNA, nuclear gene encoding chloroplast protein, | |
| | | | | | complete cds. | |
| 700281881H2 | g2275217 | 20 | 2 | gb105eukp | T08I13.24: chloroplast protein CP12 isolog | |
| 700343606H1 | g473604 | 69 | −58 | gb105pln | Zea mays W-22 histone H2B mRNA, complete cds. | |
| 700342957H1 | g1743387 | 57 | −39 | gb105pln | S. bicolor mRNA for pSbaNS5 protein. | |
| 700346949H1 | g2252639 | 17 | −15 | gb105pln | Genomic sequence of Arabidopsis BAC F8A5, complete sequence. | |
| 700346664H1 | g218132 | 64 | −55 | gb105pln | Rice mRNA for Heat shock protein 83. | |
| 700342494H1 | g2384690 | 14 | −1 | gb105allp | proteosome major subunit | |
| 700343409H1 | g485946 | 36 | −27 | gb105pln | N. tabacum NeIF-4A6 mRNA. | |
| 700343558H1 | g1777929 | 48 | −65 | gb105pln | Saccharum officinarum nucleoside diphosphate kinase (SoNDPK1) mRNA, | |
| | | | | | complete cds. | |
| 700346957H1 | g1657767 | 22 | −8 | gb105eukp | gag | |
| 700346579H1 | g1236706 | 27 | −9 | gb105allp | prolidase | |
| 700343490H1 | g20255 | 43 | −14 | gb105pln | O. sativa gene for heat shock protein 82 HSP82. | |
| 700345738H1 | g2149639 | 28 | −22 | gb105pln | Arabidopsis thaliana Argonaute protein (AGO1) mRNA, complete cds. | |
| 700380490H1 | g902526 | 26 | −3 | gb105pln | Zea mays clone MubG7 ubiquitin fusion protein gene, complete cds. | |
| 700345188H1 | g603509 | 9 | 8 | gb105eukp | cyclin delta-3 | |
| 700347368H1 | g2370518 | 5 | 4 | gb105eukp | SPAC2E11.04; ribosomal protein | |
| 700343209H1 | g2635323 | 15 | 1 | gb105allp | similar to DNA mismatch repair protein | |
| 700347014H1 | g644491 | 74 | −26 | gb105pln | Corn mRNA for elongation factor 1A. | |
| 700342313H1 | g971283 | 40 | −29 | gb105pln | Rice mRNA for ribosomal protein S31, complete cds. | |
| 700346661H1 | g1136121 | 50 | −40 | gb105pln | O. sativa mRNA for alpha-tubulin (clone OSTA-136). | |
| 700344281H1 | g871985 | 29 | −16 | gb105pln | A. sativa Aspk11 mRNA. | |
| 700346287H1 | g1322276 | 25 | 3 | gb105pln | Triticum aestivum histone H2A gene, complete cds. | |
| 700380523H1 | g289769 | 52 | −22 | gb105eukp | ZK652.3 protein | |
| 700342236H1 | g2317715 | 39 | −21 | gb105eukp | helD; helD | |
| 700343221H1 | g2160300 | 33 | −17 | gb105eukp | ribosomal protein | |
| 700347365H1 | g16732 | 24 | −10 | gb105allp | NADH ubiquinone oxidoreductase subunit | |
| 700346872H1 | g940406 | 17 | 1 | gb105allp | mcdc21 protein | |
| 700346607H1 | g1628193 | 8 | 5 | gb105allp | T12D8.3 | |
| 700343176H1 | g1495272 | 17 | 6 | gb105pln | A. thaliana mRNA for sugar transporter. | |
| 700281956H2 | g968901 | 32 | −29 | gb105pln | Rice mRNA for ribosomal protein S8, complete cds. | |
| 700344266H1 | g1244773 | 19 | −11 | gb105eukp | RPL37A; Rp137ap: 60S ribosomal protein L37a | |
| 700342796H1 | g1132482 | 50 | −34 | gb105pln | Rice mRNA for ADP-ribosylation factor, complete cds. | |
| 700380614H1 | g2662342 | 80 | −78 | gb105pln | Oryza sativa mRNA for EF-1 alpha, complete cds. | |
| 700345647H1 | g2414629 | 11 | 7 | gb105eukp | SPAC2C4.18; hypothetical protein | |
| 700342423H1 | g1906825 | 56 | −10 | gb105pln | A. thaliana hsp81.2 gene. | |
| 700345803H1 | g1777929 | 79 | −67 | gb105pln | Saccharum officinarum nucleoside diphosphate kinase (SoNDPK1) mRNA, | |
| | | | | | complete cds. | |
| 700345662H1 | g643596 | 85 | −75 | gb105pln | Corn mRNA for cysteine proteinase, clone CCP, complete cds. | |
| 700342844H1 | g1550813 | 38 | −48 | gb105pln | Z. mays mRNA for acidic ribosomal protein P0. | |
| 700342284H1 | g1906838 | 24 | −7 | gb105eukp | C56G7.1 | |
| 700344425H1 | g1381140 | 16 | 6 | gb105eukp | cul-4; CUL-4 | |
| 700346009H1 | g561663 | 44 | −32 | gb105pln | Rice mRNA, partial homologous to ribosomal protein coding sequence. | |
| 700342593H1 | g474006 | 49 | −50 | gb105pln | Rice mRNA, partial homologous to ribosomal protein S11 gene. | |
| 700344430H1 | g22322 | 30 | −36 | gb105pln | Z. mays mRNA for H2B histone (clone cH2B214). | |
| 700379779H1 | g1244652 | 44 | −20 | gb105pln | Zea mays copia-type retroelement PREM-2 gag gene, complete cds. | |
| 700379604H1 | g2736146 | 25 | −12 | gb105pln | Arabidopsis thaliana fatty acid hydroxylase Fah1p (FAH1) mRNA, complete cds. | |
| 700345709H1 | g248336 | 79 | −84 | gb105pln | polyubiquitin [maize, Genomic, 3841 nt]. | |
| 700346136H1 | g1532072 | 56 | −51 | gb105pln | Z. mays mRNA for S-adenosylmethionine decarboxylase. | |
| 700345874H1 | g21399 | 39 | −11 | gb105pln | Solanum tuberosum processed actin pseudogene. | |
| 700380138H1 | g664900 | 17 | −27 | gb105pln | C. plantagineum tkt3 gene for transketolase. | |
| 700343919H1 | g22322 | 100 | −90 | gb105pln | Z. mays mRNA for H2B histone (clone cH2B214). | |
| 700347165H1 | g4199 | 22 | −6 | gb105eukp | IPP1 | |
| 700346645H1 | g458426 | 20 | −11 | gb105eukp | dihydrolipoamide acetyltransferase | |
| 700343589H1 | g168505 | 44 | −42 | gb105pln | Zea mays histone H3 gene, complete cds. | |
| 700380042H1 | g1644402 | 17 | 0 | gb105allp | putative ATP/GTP-binding protein | |
| 700379926H1 | g1212995 | 70 | −61 | gb105pln | H. vulgare mRNA for UDP-glucose pyrophosphorylase. | |
| 700345278H1 | g2668745 | 65 | −57 | gb105pln | Zea mays inorganic pyrophosphatase (IPP) mRNA, complete cds. | |
| 700347107H1 | g168539 | 75 | −28 | gb105pln | Zea mays putative ribosomal protein S22 homolog mRNA, partial cds. | |
| 700347229H1 | g1839188 | 19 | 3 | gb105eukp | RHD3; putative GTP-binding protein; root hair defective 3 | |
| 700343880H1 | g474007 | 40 | 16 | gb105pln | Rice mRNA, partial homologous to ribosomal protein S12 gene. | |
| 700344993H1 | g951335 | 24 | −1 | gb105pln | Oryza sativa protein phosphatase 1 mRNA, complete cds. | |
| 700345801H1 | g1469220 | 23 | −1 | gb105pln | B. oleracea mRNA (unknown). | |
| 700342935H1 | g2661840 | 30 | −17 | gb105eukp | adk; adenosine kinase; EC 2.7.1.20 | |
| 700380178H1 | g1498052 | 57 | −64 | gb105pln | Zea mays ribosomal protein S8 mRNA, complete cds. | |
| 700346591H1 | g2058313 | 15 | −7 | gb105eukp | CCR1; cinnamoyl-CoA reductase; EC 1.2.1.44 | |
| 700345744H1 | g170746 | 73 | −73 | gb105pln | Wheat histone H4 TH091 gene, complete cds. | |
| 700342546H1 | g493574 | 18 | −13 | gb105eukp | BIN2; Bin2p | |
| 700380535H1 | g2341023 | 43 | −39 | gb105pln | Sequence of BAC F19P19 from Arabidopsis thaliana chromosome 1, complete sequence. | |
| 700345631H1 | g166418 | 15 | 4 | gb105allp | putative endomembrane protein; putative | |
| 700342206H1 | g2624416 | 39 | −22 | gb105pln | Zea mays mRNA for ubiquitin carrier protein UBC7. | |
| 700343922H1 | g16086 | 43 | −34 | gb105pln | A. porrum dnaJ mRNA for DNA J protein (partial). | |
| 700345438H1 | g836941 | 41 | −28 | gb105pln | Arabidopsis thaliana calcium-dependent protein kinase (CDPK19) mRNA, complete cds. | |
| 700379601H1 | g1906827 | 39 | −17 | gb105pln | A. thaliana hsp81.4 gene. | |
| 700346110H1 | g20588 | 21 | −11 | gb105pln | Pea PU1 gene mRNA for polyubiquitin. | |
| 700343889H1 | g2623679 | 71 | −67 | gb105pln | Zea mays calmodulin (Zmrcalm) mRNA, complete cds. | |
| 700343017H1 | g19070 | 51 | −38 | gb105pln | H. vulgare pazx gene encoding protein zx. | |
| 700343057H1 | g972931 | 32 | −12 | gb105eukp | IAA14; IAA14 | |
| 700344266H1 | g4392 | 19 | −11 | gb105eukp | RPL37A; ribosomal protein L37a | |
| 700342551H1 | g1710151 | 31 | −13 | gb105eukp | proline iminopeptidase | |
| 700281872H2 | g1255684 | 64 | −6 | gb105pln | Ric emRNA for aspartic protease, complete cds. | |
| 700343921H1 | g166810 | 21 | −6 | gb105pln | Arabidopsis thaliana Columbia protein kinase mRNA, complete cds. | |
| 700380294H1 | g2244788 | 11 | 17 | gb105pln | Arabidopsis thaliana DNA chromosome 4, ESSA I contig fragment No. 1. | |
| 700342488H1 | g22155 | 43 | −18 | gb105pln | Z. mays mRNA for alpha-tubulin 5. | |
| 700345002H1 | g2264307 | 16 | 6 | gb105pln | Arabidopsis thaliana genomic DNA, chromosome 5, P1 clone: MED24, complete | |
| | | | | | sequence. | |
| 700379623H1 | g2245031 | 12 | 5 | gb105pln | Arabidopsis thaliana DNA chromosome 4, ESSA I contig fragment No. 7. | |
| 700345696H1 | g1017825 | 34 | 2 | gb105allp | RNA polymerase II subunit | |
| 700343053H1 | g2149018 | 17 | −28 | gb105pln | Oryza sativa putative ADP-glucose pyrophosphyorylase subunit SH2 and putative | |
| | | | | | NADPH-dependent reductase A1 genes, complete cds. | |
| 700344751H1 | g498772 | 25 | −67 | gb105pln | Z. mays (cv DH5xDH7) hsp70-4 mRNA for heat shock protein 70. | |
| 700346621H1 | g452445 | 27 | −28 | gb105pln | Arabidopsis thaliana Landsberg erecta cycloartenol synthase (CAS1) mRNA, | |
| | | | | | complete cds. | |
| 700342374H1 | g1905943 | 59 | −47 | gb105pln | Sorghum bicolor MADS box transcription factor SbMADS1 mRNA, complete cds. | |
| 700379728H1 | g397400 | 36 | −32 | gb105pln | B. rapa mRNA for S phase specific gene. | |
| 700346426H1 | g493589 | 11 | 6 | gb105eukp | PDI; catalyze the formation of disulfide bonds; disulfide isomerase; EC 5.3.4.1 | |
| 700345587H1 | g1890574 | 63 | −50 | gb105pln | H. vulgare mRNA for xyloglucan endotransglycosylase-like protein (XEA). | |
| 700281913H2 | g169778 | 40 | −41 | gb105pln | Oryza sativa beta-amylase gene, complete cds. | |
| 700346391H1 | g416146 | 45 | −17 | gb105pln | Zea mays beta-6 tubulin (tub6) gene and mRNA, complete cds. | |
| 700345958H1 | g1574937 | 28 | −3 | gb105pln | Zea mays superoxide dismutase 4 (sod4) gene, partial cds. | |
| 700342518H1 | g2443314 | 72 | −36 | gb105allp | keratin 14 | |
| 700346250H1 | g510676 | 18 | −24 | gb105pln | Triticum aestivium pWR5 RNA for protochlorophyilide reductase. | |
| 700343210H1 | g303854 | 47 | −52 | gb105pln | Rice mRNA for ribosomal protein L7A, complete cds. | |
| 700347234H1 | g1903347 | 17 | −0 | gb105eukp | F19P19.6 | |
| 700347169H1 | g1255951 | 23 | −7 | gb105eukp | PS60 | |
| 700346478H1 | g2511540 | 13 | 15 | gb105pln | Oryza sativa DNA-binding protein GBP16 (Rgbp16) mRNA, complete cds. | |
| 700344325H1 | g42223 | 9 | 5 | gb105allp | P35 gene product (AA 1-314) | |
| 700344705H1 | g1139591 | 7 | 6 | gb105eukp | VCX1; vacuolar H+/Ca2+ exchanger | |
| 700343736H1 | g683503 | 30 | −17 | gb105pln | A. thaliana mRNA for 65 kDa regulatory subunit of protein phosphatase 2A. | |
| 700281965H1 | g2852448 | 22 | −8 | gb105pln | Arabidopsis thaliana APK2b mRNA for protein kinase, complete cds. | |
| 700379620H1 | g474009 | 71 | −61 | gb105pln | Rice mRNA, partial homologous to ribosomal protein S19 gene. | |
| 700342691H1 | g168487 | 59 | −53 | gb105pln | Maize glutathione S-transferase gene (GST-I), exons 2 and 3. | |
| 700343878H1 | g2645166 | 32 | −3 | gb105pln | Oryza sativa mRNA, similar to ubiquitin conjugating enzyme. | |
| 700344612H1 | g393400 | 85 | −74 | gb105pln | Z. mays mRNA for alpha-tubulin. | |
| 700342782H1 | g2275202 | 18 | −7 | gb105eukp | T08I13.8; acyl-CoA synthetase isolog | |
| 700343244H1 | g2662468 | 41 | −24 | gb105pln | Arabidopsis thaliana ribosomal protein S6 (rps6) mRNA, complete cds. | |
| 700344784H1 | g1248427 | 14 | 7 | gb105allp | subtilisin | |
| 700281947H2 | g2511530 | 44 | −43 | gb105pln | Eleusine indica alpha tubulin 1 (TUA1) mRNA, complete cds. | |
| 700343907H1 | g1301851 | 26 | −13 | gb105eukp | HDA1 | |
| 700379660H1 | g19342 | 22 | −10 | gb105pln | L. esculentum mRNA for ribosomal protein L2. | |
| 700342715H1 | g2315459 | 10 | −1 | gb105eukp | F41E6.13 | |
| 700344443H1 | g1872162 | 10 | 16 | gb105pln | Arabidopsis thaliana DnaJ homolog (atj) mRNA, complete cds. | |
| 700345874H1 | g1002532 | 26 | −11 | gb105pln | Arabidopsis thaliana actin-11 (ACT11) gene, complete cds. | |
| 700380124H1 | g2098574 | 12 | 5 | gb105allp | COXG | |
| 700344686H1 | g2696081 | 10 | 16 | gb105pln | Arabidopsis thaliana genomic DNA, chromosome 5, P1 clone: MXC9. | |
| 700342928H1 | g1129085 | 41 | −22 | gb105pln | Wheat mRNA for protein H2A, complete cds, clone wcH2A-9. | |
| 700344810H1 | g167244 | 35 | −24 | gb105pln | Chlorella kessleri elongation factor 2 mRNA, complete cds. | |
| 700380075H1 | g577824 | 18 | −23 | gb105pln | Z. mays gene for H2B histone (gH2B3). | |
| 700379913H1 | g2160172 | 30 | 2 | gb105eukp | F21M12.20 | |
| 700346392H1 | g553063 | 12 | 5 | gb105eukp | GST2; glutathione s-transferase | |
| 700347215H1 | g960289 | 28 | −19 | gb105eukp | anthranilate synthase alpha subunit | |
| 700344941H1 | g1632821 | 30 | −30 | gb105pln | O. sativa mRNA for transmembrane protein. | |
| 700344942H1 | g710431 | 17 | 6 | gb105pln | Glycine max low molecular weight heat shock protein Hsp23.9 (Gmhsp23.9) mRNA, | |
| | | | | | complete cds. | |
| 700380003H1 | g180941 | 56 | −37 | gb105allp | cytochrome c oxidase precursor (EC 1.9.3.1) | |
| 700346088H1 | g1181272 | 6 | 7 | gb105eukp | L2128 | |
| 700342902H1 | g886739 | 39 | −48 | gb105pln | Z. mays histone H4 gene. | |
| 700344281H1 | g508307 | 9 | 14 | gb105pln | Arabidopsis thaliana protein-serine kinase mRNA, complete cds. | |
| 700342270H1 | g1524120 | 31 | −15 | gb105pln | M. crystallinum mRNA for malate dehydrogenase. | |
| 700346863H1 | g2764573 | 10 | 15 | gb105pln | Pisum sativum mRNA for pore protein of 24 kD OEF24. | |
| 700345734H1 | g2282583 | 45 | −36 | gb105pln | Zea mays elongation factor 1-alpha (EF1-A) mRNA, complete cds. | |
| 700345348H1 | g1184773 | 97 | −79 | gb105pln | Zea mays glyceraldehyde-3-phosphate dehydrogenase GAPC3 (gpc3) mRNA, | |
| | | | | | complete cds. | |
| 700343506H1 | g2274990 | 20 | −41 | gb105pln | Hordeum vulgare mRNA for expressed sequence tag. | |
| 700343586H1 | g2316063 | 12 | −11 | gb105eukp | cyt-b; cytochrome apoenzyme b | |
| 700342330H1 | g2407278 | 11 | 5 | gb105pln | Oryza sativa aldolase mRNA, complete cds. | |
| 700344422H1 | g2661193 | 21 | −9 | gb105eukp | rp15; ribosomal protein L5 | |
| 700345628H1 | g169295 | 34 | −14 | gb105pln | Pharbitis nil heat shock protein 83 (Hsp83) gene, complete cds. | |
| 700342489H1 | g2257526 | 15 | −0 | gb105eukp | pi040; PROTEIN-TYROSINE PHOSPHATASE YVH1 | |
| 700380791H1 | g13483 | 21 | 4 | gb105allp | ATPase subunit 6 | |
| 700345416H1 | g20000 | 33 | −31 | gb105pln | N. tabacum RL2 mRNA for 60S ribosomal protein L2. | |
| 700342755H1 | g1553130 | 15 | 17 | gb105pln | Gossypium hirsutum ribosomal protein L44 isoform b (RL44), complete cds. | |
| 700380428H1 | g887793 | 67 | −33 | gb105allp | FXR1 | |
| 700379832H1 | g2661839 | 30 | −18 | gb105pln | Physcomitrella patens mRNA for adenosine kinase. | |
| 700346908H1 | g829147 | 100 | −84 | gb105pln | Z. mays gene for cyclophilin. | |
| 700379706H1 | g2814825 | 9 | 7 | gb105eukp | T10C6.12 | |
| 700346709H1 | g1944508 | 45 | −40 | gb105pln | Spinacia oleracea mRNA for stromal ascorbate peroxidase, complete cds. | |
| 700346005H1 | g972205 | 37 | 4 | gb105allp | 60S ribosomal protein P1 | |
| 700343258H1 | g1321924 | 7 | 6 | gb105allp | NAM gene product | |
| 700345650H1 | g2564051 | 27 | −20 | gb105pln | Arabidopsis thaliana genomic DNA, chromosome 5, P1 clone: MWD9, complete | |
| | | | | | sequence. | |
| 700345637H1 | g173231 | 23 | −6 | gb105pln | Yeast (Saccharomyces cerevisiae) ribosomal 5S RNA-binding protein (YL3) gene, 5′ cds. | |
| 700347208H1 | g763271 | 10 | 7 | gb105allp | Sen3p | |
| 700342393H1 | g541611 | 47 | −33 | gb105allp | cytokeratin 13 | |
| 700343811H1 | g634003 | 26 | −14 | gb105eukp | acyl-(acyl carrier protein) thioesterase; EC 3.1.2.14 | |
| 700346940H1 | g1209703 | 35 | −16 | gb105eukp | maize g11 homolog | |
| 700346440H1 | g218088 | 54 | −69 | gb105pln | Rice mRNA for ribosomal protein 117 (249 gene), partial sequence. | |
| 700343894H1 | g2511530 | 78 | −39 | gb105pln | Eleusine indica alpha tubulin 1 (TUA1) mRNA, complete cds. | |
| 700380255H1 | g2662346 | 56 | −47 | gb105pln | Oryza sativa mRNA for EF-1 alpha, complete cds. | |
| 700342311H1 | g1519400 | 44 | −36 | gb105pln | Chlorella vulgaris actin mRNA, partial cds. | |
| 700344409H1 | g168543 | 51 | −34 | gb105pln | Zea mays putative ribosomal protein S8 mRNA, partial cds. | |
| 700380278H1 | g2828011 | 11 | 15 | gb105pln | Zea mays starch synthase I precursor (Ss1) mRNA, nuclear gene encoding plastid protein, | |
| | | | | | complete cds. | |
| 700346948H1 | g395076 | 44 | −33 | gb105pln | B. rapa ribosomal protein L37a mRNA, complete CDS. | |
| 700342962H1 | g1370145 | 17 | 7 | gb105pln | L. japonicus mRNA for small GTP-binding protein, RAB11C. | |
| 700342826H1 | g968901 | 56 | −41 | gb105pln | Rice mRNA for ribosomal protein S8, complete cds. | |
| 700343236H1 | g20163 | 32 | −21 | gb105pln | O. sativa Rr15 mRNA for 5S ribosomal RNA. | |
| 700343711H1 | g1008175 | 21 | −2 | gb105eukp | ORF YJL045w | |
| 700346430H1 | g1935046 | 10 | 17 | gb105pln | Zea mays helix-loop-helix type transcription factor R (R-d:Catspaw allele) gene, exon 1, | |
| | | | | | inverted repeat, partial cds. | |
| 700342505H1 | g2130984 | 38 | −24 | gb105pln | B. napus mRNA for ribosomal protein S15a (pA813). | |
| 700342288H1 | g1905929 | 17 | −6 | gb105pln | Oryza sativa MADS box protein OsMADS45 mRNA, complete cds. | |
| 700344447H1 | g1420885 | 16 | −7 | gb105eukp | proline-rich 14 kDa protein | |
| 700345302H1 | g304668 | 23 | −2 | gb105eukp | ben | |
| 700346106H1 | g2599103 | 19 | 3 | gb105pln | Dunaliella salina 60S ribosomal protein (DSRP1) mRNA, complete cds. | |
| 700347139H1 | g1835730 | 13 | 17 | gb105pln | Oryza sativa photosystem II 10 kDa polypeptide mRNA, complete cds. | |
| 700343736H1 | g437900 | 26 | −16 | gb105pln | P. sativum mRNA for phosphoprotein phosphatase 2A 65 kDa regulatory subunit. | |
| 700346441H1 | g1654002 | 8 | 3 | gb105allp | Sop2p-like protein | |
| 700345667H1 | g2463334 | 52 | −44 | gb105pln | Oryza sativa mRNA for ribosomal protein S4. | |
| 700345584H1 | g849185 | 8 | 7 | gb105eukp | CDC40; Cdc40p | |
| 700344724H1 | g2829133 | 13 | 2 | gb105eukp | akn2; phosphorylates 3′-OH group of adenosine-5′-phosphosulfate; adenosine-5′- | |
| | | | | | phosphosulfate-kinase; EC 2.7.1.25 | |
| 700346406H1 | g19538 | 9 | 9 | gb105pln | M. crystallinum mRNA for ribosomal protein YL16. | |
| 700281967H1 | g533251 | 54 | −78 | gb105pln | Zea mays (clone pSM8) sucrose synthase 2 (Sus1) gene, complete cds. | |
| 700346337H1 | g21800 | 37 | −35 | gb105pln | T. aestivum L mRNA for histone H2B. | |
| 700343962H1 | g2425065 | 90 | −83 | gb105pln | Zea mays cysteine proteinase Mir3 (mir3) mRNA, complete cds. | |
| 700379637H1 | g2673869 | 29 | −12 | gb105pln | Antirrhinum majus mRNA for fimbriata-associated protein 2, partial. | |
| 700342536H1 | g168500 | 50 | −47 | gb105pln | Maize (Zea mays) histone H4 gene (H4C14), complete cds. | |
| 700344630H1 | g2088647 | 27 | −16 | gb105eukp | T28M21.10 | |
| 700281804H2 | g2739218 | 42 | −31 | gb105pln | Hordeum vulgare S28 ribosomal protein. | |
| 700343480H1 | g312345 | 12 | 4 | gb105allp | rat ribosomal protein L36 | |
| 700380164H1 | g22272 | 95 | −89 | gb105pln | Maize mRNA for enolase (2-phospho-D-glycerate hydrolase). | |
| 700281934H2 | g17645 | 56 | −3 | gb105allp | RIBOSOMAL PROTEIN L35a | |
| 700344241H1 | g20760 | 18 | 13 | gb105pln | P. sativum gTUB3 mRNA for beta-tubulin 3. | |
| 700344893H1 | g22293 | 7 | 5 | gb105eukp | glycine-rich protein | |
| 700380711H1 | g2286152 | 74 | −78 | gb105pln | Zea mays cytoplasmic malate dehydrogenase mRNA, complete cds. | |
| 700346910H1 | g1130617 | 28 | −3 | gb105eukp | F11A10.2 | |
| 700343858H1 | g22375 | 10 | 4 | gb105eukp | ORF | |
| 700344193H1 | g309666 | 12 | 6 | gb105allp | electron transfer flavoprotein alpha-subunit | |
| 700345065H1 | g1906827 | 47 | −57 | gb105pln | A. thaliana hsp81.4 gene. | |
| 700343661H1 | g450548 | 29 | −22 | gb105pln | O. sativa (pRSAM-1) gene for S-adenosyl methionine synthetase. | |
| 700347290H1 | g1020408 | 100 | −82 | gb105pln | Zea mays mRNA for phospholipase D, complete cds. | |
| 700343113H1 | g2656032 | 34 | −13 | gb105pln | Arabidopsis thaliana genomic DNA, chromosome 5, P1 clone: MZF18. | |
| 700345262H1 | g600768 | 37 | −22 | gb105pln | Oryza sativa cyclophilin 2 (Cyp2) mRNA, complete cds. | |
| 700342533H1 | g386764 | 65 | −8 | gb105allp | hba2 alpha globin | |
| 700343411H1 | g1052973 | 21 | −9 | gb105eukp | fructokinase; EC 2.7.1.4 | |
| 700346548H1 | g1785691 | 11 | 5 | gb105allp | ribosomal protein L5 | |
| 700379968H1 | g2570508 | 40 | −17 | gb105pln | Oryza sativa transmembrane protein mRNA, complete cds. | |
| 700343013H1 | g2599103 | 20 | −4 | gb105pln | Dunaliella salina 60S ribosomal protein (DSRP1) mRNA, complete cds. | |
| 700343846H1 | g2274990 | 51 | −41 | gb105pln | Hordeum vulgare mRNA for expressed sequence tag. | |
| 700345337H1 | g1665866 | 24 | −3 | gb105pln | C. calcitrapa mRNA for aspartic proteinase. | |
| 700346373H1 | g1890576 | 24 | −33 | gb105pln | H. vulgare mRNA for xyloglucan endotransglycosylase-like protein (XEB). | |
| 700344194H1 | g1526412 | 32 | −19 | gb105pln | Arabidopsis thaliana ATPK19 mRNA for ribosomal-protein S6 kinase homolog, | |
| | | | | | complete cds. | |
| 700344516H1 | g487932 | 23 | −9 | gb105eukp | YHR088w; Yhr088wp | |
| 700343309H1 | g386764 | 60 | −27 | gb105allp | hba2 alpha globin | |
| 700342532H1 | g1061077 | 7 | 6 | gb105allp | R07A4.1 | |
| 700347319H1 | g348695 | 25 | −11 | gb105pln | Catharanthus roseus heat shock protein 90 mRNA, complete cds. | |
| 700345255H1 | g2739371 | 18 | −4 | gb105eukp | T9J22.13 | |
| 700380141H1 | g55775 | 42 | 4 | gb105allp | alpha-tubulin | |
| 700342847H1 | g2656031 | 16 | 12 | gb105pln | Arabidopsis thaliana genomic DNA, chromosome 5, P1 clone: MXC20. | |
| 700343160H1 | g249261 | 14 | 10 | gb105pln | linamarase = beta-glucosidase [Manihot esculenta = cassava, Cranz, mRNA, 1705 nt]. | |
| 700343414H1 | g558651 | 25 | −5 | gb105pln | T. aestivum VDAC3 mRNA for voltage dependent anion channel. | |
| 700344004H1 | g2642216 | 33 | 3 | gb105pln | Oryza sativa NOI protein mRNA, complete cds. | |
| 700344822H1 | g2369713 | 23 | −34 | gb105pln | Beta vulgaris cDNA for elongation factor 2. | |
| 700347170H1 | g2811175 | 38 | −66 | gb105pln | Zea mays alternative oxidase (Aox) gene, partial cds. | |
| 700343763H1 | g1149569 | 13 | −7 | gb105eukp | athb-8; HD-zip | |
| 700379678H1 | g473602 | 50 | −76 | gb105pln | Zea mays W-22 histone H2A mRNA, complete cds. | |
| 700342344H1 | g1402896 | 19 | 2 | gb105eukp | cyclin delta-2 | |
| 700344729H1 | g1911548 | 21 | 2 | gb105allp | cytochrome c-like polypeptide [human, lung adenocarcinoma A549, Peptide, 190 aa] | |
| 700342283H1 | g2583081 | 8 | 4 | gb105allp | microsomal glutathione S-transferase 3 | |
| 700346622H1 | g2662342 | 62 | −48 | gb105pln | Oryza sativa mRNA for EF-1 alpha, complete cds. | |
| 700343251H1 | g169537 | 28 | −15 | gb105pln | Potato pyrophosphate-fructose 6-phosphate 1-phosphotransferase (PFP) alpha-subunit | |
| | | | | | mRNA, complete cds. | |
| 700346816H1 | g1773171 | 13 | 2 | gb105allp | similar to M. tuberculosis MTCY277.09 | |
| 700345984H1 | g168502 | 31 | 8 | gb105pln | Maize (Zea mays) histone H4 gene (H4C7), complete cds. | |
| 700343204H1 | g1118105 | 26 | −1 | gb105eukp | K02B2.5 | |
| 700343886H1 | g2160295 | 31 | −20 | gb105eukp | APK1b; protein tyrosine-serine-threonine kinase | |
| 700344251H1 | g940446 | 20 | 0 | gb105eukp | CYP86; cytochrome p450 | |
| 700347378H1 | g19102 | 31 | −19 | gb105eukp | Cytoplasmic large subunit ribosomal protein; ribosomal protein L17-1 | |
| 700343128H1 | g1129083 | 16 | 7 | gb105pln | Wheat mRNA for protein H2A, complete cds, clone wcH2A-2. | |
| 700379661H1 | g547477 | 24 | −3 | gb105pln | V. faba mRNA for guanine nucleotide regulatory protein. | |
| 700347285H1 | g972919 | 18 | 2 | gb105eukp | IAA8; IAA8 | |
| 700342647H1 | g600115 | 33 | −10 | gb105pln | Z. mays apx gene encoding cytosolic ascorbate peroxidase. | |
| 700344631H1 | g1064924 | 18 | −0 | gb105pln | N. tabacum mRNA for cyclin A-like protein (clone 105). | |
| 700345809H1 | g170746 | 12 | 16 | gb105pln | Wheat histone H4 TH091 gene, complete cds. | |
| 700380716H1 | g28547 | 40 | −22 | gb105allp | alpha globin | |
| 700379735H1 | g2369713 | 37 | −26 | gb105pln | Beta vulgaris cDNA for elongation factor 2. | |
| 700346857H1 | g1184596 | 13 | 3 | gb105eukp | T11G6.3 | |
| 700343531H1 | g1658312 | 16 | −12 | gb105pln | O. sativa osr40g2 gene. | |
| 700345761H1 | g1706957 | 43 | −11 | gb105pln | Gossypium hirsutum cellulose synthase (celA2) mRNA, partial cds. | |
| 700343711H1 | g407500 | 18 | −0 | gb105eukp | unknown | |
| 700343038H1 | g1778377 | 28 | −20 | gb105pln | Pisum sativum NAP1Ps mRNA, complete cds. | |
| 700342257H1 | g2760173 | 23 | −6 | gb105pln | Arabidopsis thaliana genomic DNA, chromosome 5, P1 clone: MYH19, complete | |
| | | | | | sequence. | |
| 700344302H1 | g577824 | 27 | −27 | gb105pln | Z. mays gene for H2B histone (gH2B3). | |
| 700343953H1 | g1498052 | 60 | −54 | gb105pln | Zea mays ribosomal protein S8 mRNA, complete cds. | |
| 700346083H1 | g556672 | 31 | −14 | gb105pln | S. cereale (Halo) chloroplast mRNA for heat-shock protein. | |
| 700342516H1 | g435172 | 70 | −62 | gb105pln | A. sativa (Pewi) ASTCP-K19 mRNA for t complex polypeptide 1. | |
| 700342440H1 | g7117 | 28 | −29 | gb105eukp | ribosomal YL10 protein homologue | |
| 700343055H1 | g2239259 | 96 | −25 | gb105pln | Zea mays mRNA for cinnamoyl CoA reductase. | |
| 700345564H1 | g2827698 | 21 | 1 | gb105pln | Arabidopsis thaliana DNA chromosome 4, BAC clone F6H11 (ESSAI project). | |
| 700345628H1 | g300082 | 26 | −4 | gb105pln | hsp82 = 82 kda heat shock protein [Zea mays, seedling, leaves, Genomic, 3468 nt]. | |
| 700345515H1 | g2191126 | 12 | 15 | gb105pln | Arabidopsis thaliana BAC IG002N01. | |
| 700342866H1 | g1658341 | 60 | −5 | gb105eukp | T22F3.4 | |
| 700343928H1 | g2213424 | 37 | −28 | gb105pln | Citrus paradisi mRNA for hypothetical protein. | |
| 700345155H1 | g2262173 | 21 | 4 | gb105eukp | F5J6.18; NADPH thioredoxin reductase | |
| 700345951H1 | g2668743 | 53 | −21 | gb105pln | Zea mays ubiquitin conjugating enzyme (UBC) mRNA, complete cds. | |
| 700342671H1 | g2351063 | 19 | 4 | gb105pln | Arabidopsis thaliana genomic DNA, chromosome 5, P1 clone: MCL19. | |
| 700344310H1 | g2583129 | 27 | −9 | gb105eukp | F4L23.25; putative methionine aminopeptidase | |
| 700345257H1 | g337518 | 13 | 6 | gb105allp | ribosomal protein | |
| 700343778H1 | g170228 | 24 | −23 | gb105pln | N. tabacum 5-enolpyruvylshikimate-3-phosphate synthase mRNA, complete cds. | |
| 700380074H1 | g498774 | 45 | −44 | gb105pln | Z. mays (cv DH5xDH7) hsp 70-5 mRNA for heat shock protein 70. | |
| 700380479H1 | g2369810 | 14 | 8 | gb105allp | ribosomal protein S24 | |
| 700343470H1 | g20250 | 65 | −43 | gb105pln | Oryza sativa H3 histone gene H3R-11. | |
| 700345318H1 | g1597723 | 7 | 7 | gb105allp | CRINKLY4 precursor | |
| 700342825H1 | g1049252 | 60 | −23 | gb105pln | Zea mays vacuolar ATPase 69 kDa subunit mRNA, partial cds. | |
| 700342722H1 | g1354932 | 14 | 10 | gb105pln | Olea europaeafruit stearoyl-ACP desaturase, complete cds. | |
| 700343924H1 | g167107 | 23 | −14 | gb105pln | Hordeum vulgare vacuolar ATPase B subunit isoform mRNA, complete cds. | |
| 700344505H1 | g2098574 | 11 | 2 | gb105allp | COXG | |
| 700345259H1 | g2208914 | 7 | −1 | gb105eukp | outer arm dynein LC6 | |
| 700346341H1 | g531031 | 14 | −15 | gb105pln | Pennisetum ciliare apomixis-associated mRNA. | |
| 700380473H1 | g22121 | 58 | −59 | gb105pln | Maize alcohol dehydrogenase 1 gene (Adh1-1F). | |
| 700346011H1 | g1128947 | 18 | 2 | gb105allp | ORF X; putative | |
| 700281891H2 | g487961 | 16 | −3 | gb105eukp | YHR065c; Yhr065cp | |
| 700342855H1 | g168502 | 29 | 17 | gb105pln | Maize (Zea mays) histone H4 gene (H4C7), complete cds. | |
| 700343654H1 | g147716 | 11 | 5 | gb105allp | ribosomal protein L17 | |
| 700346661H1 | g22149 | 77 | −65 | gb105pln | Z. mays mRNA for alpha-tubulin 3. | |
| 700379778H1 | g1015460 | 25 | 1 | gb105eukp | F26C11.1 | |
| 700342419H1 | g398605 | 12 | 5 | gb105pln | A. thaliana gene for elongation factor 1 beta. | |
| 700346193H1 | g533251 | 44 | −6 | gb105pln | Zea mays (clone pSM8) sucrose synthase 2 (Sus1) gene, complete cds. | |
| 700344509H1 | g500852 | 78 | −64 | gb105pln | Zea mays (clone pAKHSDH2) aspartate kinase-homserine dehydrogenase mRNA, | |
| | | | | | complete cds. | |
| 700380003H1 | g180937 | 56 | −37 | gb105allp | COX5B | |
| 700379646H1 | g474000 | 58 | −48 | gb105pln | Rice mRNA, partial homologous to ribosomal protein L37a gene. | |
| 700345522H1 | g2695940 | 34 | −20 | gb105pln | Hordeum vulgare mRNA for ribosomal like-protein, clone RG131. | |
| 700344002H1 | g2809232 | 17 | 7 | gb105eukp | F21B7.1 | |
| 700379628H1 | g432606 | 49 | −37 | gb105pln | ric1 = ras-related GTP binding protein possessing GTPase activity [Oryza sativa = rice, | |
| | | | | | Yamahoushi, callus, mRNA, 955 nt]. | |
| 700342862H1 | g600768 | 40 | 6 | gb105pln | Oryza sativa cyclophilin 2 (Cyp2) mRNA, complete cds. | |
| 700342657H1 | g2297175 | 22 | 4 | gb105allp | PROLINE IMINOPEPTIDASE | |
| 700343648H1 | g625108 | 42 | −5 | gb105eukp | YLR388W; Ylr388wp | |
| 700342738H1 | g2318116 | 34 | −41 | gb105pln | Pisum sativum Mg-chelatase subunit D (ChlD) mRNA, complete cds. | |
| 700345111H1 | g2160162 | 22 | −14 | gb105eukp | F21M12.8 | |
| 700347314H1 | g514945 | 25 | −18 | gb105pln | Zea mays sucrose synthase (Sus1) mRNA, complete cds. | |
| 700342604H1 | g2760330 | 13 | 4 | gb105eukp | F1N21.15 | |
| 700380681H1 | g2565339 | 17 | −4 | gb105pln | Lupinus luteus ribosomal protein S14 (rps14) mRNA, complete cds. | |
| 700342374H1 | g939780 | 44 | −26 | gb105pln | Zea mays MADS-box protein (ZAG5) mRNA, complete cds. | |
| 700343502H1 | g218227 | 35 | −50 | gb105pln | Rice mRNA for ras-related GTP binding protein, complete cds. | |
| 700342210H1 | g1321917 | 47 | −18 | gb105eukp | su12; cytoplasmic ribosomal protein S7 | |
| 700281875H2 | g2827001 | 47 | −44 | gb105pln | Triticum aestivum 70 kDa heat shock protein (TaHSP70d) mRNA, complete cds. | |
| 700344826H1 | g913941 | 11 | 3 | gb105allp | aldehyde dehydrogenase homolog = btg-26 [Brassica napus, cv. Bridger, Peptide, | |
| | | | | | 494 aa] | |
| 700345682H1 | g20255 | 50 | −7 | gb105pln | O. sativa gene for heat shock protein 82 HSP82. | |
| 700345894H1 | g780371 | 41 | −18 | gb105pln | Oryza sativa enolase mRNA, complete cds. | |
| 700347277H1 | g2351065 | 12 | 13 | gb105pln | Arabidopsis thaliana genomic DNA, chromosome 5, P1 clone: MHF15. | |
| 700346159H1 | g19701 | 12 | 9 | gb105pln | N. plumbaginifolia mRNA NeIF-5A2 for initiation factor 5A(2). | |
| 700343950H1 | g552063 | 20 | 4 | gb105allp | actin | |
| 700347006H1 | g2351067 | 12 | 14 | gb105pln | Arabidopsis thaliana genomic DNA, chromosome 5, P1 clone: MPO12. | |
| 700346196H1 | g393400 | 95 | −78 | gb105pln | Z. mays mRNA for alpha-tubulin. | |
| 700344814H1 | g1103393 | 30 | −13 | gb105eukp | GCTK 2; signal transduction; Sponge receptor tyrosine kinase; EC 2.7.1.112 | |
| 700344702H1 | g493707 | 59 | −48 | gb105pln | Rice mRNA for beta-tubulin, complete cds. | |
| 700346708H1 | g397395 | 100 | −96 | gb105pln | Z. mays MNB1b mRNA for DNA-binding protein. | |
| 700346229H1 | g170224 | 43 | −35 | gb105pln | Nicotiana tabacum 3-deoxy-D-arabino-heptulosonate 7-phosphate synthase, | |
| | | | | | complete cds. | |
| 700342840H1 | g2431766 | 87 | −73 | gb105pln | Zea mays acidic ribosomal protein P3a (rpp3a) mRNA, complete cds. | |
| 700344923H1 | g1373395 | 21 | 0 | gb105pln | Arabidopsis thaliana FK506 binding protein FKBP62 (ROF1) gene, complete cds. | |
| 700346461H1 | g533251 | 54 | −57 | gb105pln | Zea mays (clone pSM8) sucrose synthase 2 (Sus1) gene, complete cds. | |
| 700344281H1 | g867996 | 9 | 14 | gb105pln | Arabidopsis thaliana ATPK6 mRNA for ribosomal-protein S6 kinase homolog, | |
| | | | | | complete cds. | |
| 700344629H1 | g1061037 | 29 | −16 | gb105pln | A. thaliana mRNA for sterol-C5-desaturase. | |
| 700380456H1 | g1881694 | 44 | −73 | gb105pln | Zea mays phosphoglucomutase mRNA, partial cds. | |
| 700346214H1 | g2358139 | 14 | −5 | gb105pln | Arabidopsis thaliana chromosome 1 YAC yUP8H12 complete sequence. | |
| 700342292H1 | g2662342 | 59 | −45 | gb105pln | Oryza sativa mRNA for EF-1 alpha, complete cds. | |
| 700346487H1 | g642133 | 28 | −10 | gb105pln | Arabidopsis thaliana AME3 mRNA for protein kinase, complete cds. | |
| 700346469H1 | g21800 | 22 | −27 | gb105pln | T. aestivum L mRNA for histone H2B. | |
| 700343280H1 | g425196 | 12 | 2 | gb105allp | adenylate kinase | |
| 700345521H1 | g1143391 | 25 | −12 | gb105pln | A. thaliana mRNA for uridine diphosphate glucose epimerase. | |
| 700344607H1 | g20321 | 13 | 3 | gb105pln | Oryza sativa Rac1 mRNA for actin. | |
| 700380171H1 | g1911582 | 11 | 5 | gb105eukp | Cyn d 1; Cyn d 1 | |
| 700345302H1 | g546724 | 23 | −2 | gb105eukp | ben | |
| 700343711H1 | g172551 | 18 | −0 | gb105eukp | SDH1; succinate dehydrogenase flavoprotein subunit | |
| 700344389H1 | g940843 | 8 | 1 | gb105allp | orf 06116 | |
| 700346921H1 | g168500 | 61 | −43 | gb105pln | Maize (Zea mays) histone H4 gene (H4C14), complete cds. | |
| 700346824H1 | g297889 | 34 | −9 | gb105eukp | B2 protein | |
| 700346546H1 | g1325967 | 21 | −4 | gb105pln | T. aestivum histone H2A gene (clone TH274). | |
| 700342243H1 | g2511572 | 21 | 1 | gb105eukp | prcga; multicatalytic endopeptidase; EC 3.4.99.46 | |
| 700345962H1 | g1429287 | 9 | 5 | gb105allp | F58H1.3 | |
| 700342706H1 | g303852 | 56 | −69 | gb105pln | Rice mRNA for ribosomal protein L3, complete cds. | |
| 700343377H1 | g407800 | 24 | 6 | gb105pln | G. hirsutum mRNA for ribosomal protein 41, large subunit (RL41). | |
| 700347174H1 | g532571 | 39 | −25 | gb105pln | Barley lipoxygenase 1 (LoxA) gene, complete cds. | |
| 700344915H1 | g2191149 | 27 | −13 | gb105eukp | A_IG002N01.22 | |
| 700346915H1 | g2739197 | 6 | 5 | gb105eukp | pinA; peptidyl-prolyl cis/trans isomerase | |
| 700345661H1 | g1405747 | 17 | −0 | gb105allp | PR264/SC35 | |
| 700379665H1 | g1321660 | 48 | −29 | gb105pln | Rice mRNA for ascorbate peroxidase, complete cds. | |
| 700345752H1 | g2737891 | 26 | −14 | gb105pln | Sartorya fumigata nucleolar protein AfCbf5p (AfCBF5p) mRNA, complete cds. | |
| 700344634H1 | g577187 | 28 | −10 | gb105eukp | CDC46; Cdc46p | |
| 700343909H1 | g1872162 | 27 | −7 | gb105pln | Arabidopsis thaliana DnaJ homolog (atj) mRNA, complete cds. | |
| 700345539H1 | g168406 | 13 | 16 | gb105pln | Z. mays alcohol dehydrogenase (ADH-1 C-m allele) gene, complete cds. | |
| 700344601H1 | g908831 | 36 | −13 | gb105eukp | C09H10.3 | |
| 700342311H1 | g1906606 | 47 | −40 | gb105pln | Cyanophora paradoxa actin gene, partial cds. | |
| 700343416H1 | g1707006 | 26 | −11 | gb105pln | Arabidopsis thaliana chromosome II BAC T01B08 genomic sequence, complete | |
| | | | | | sequence. | |
| 700342722H1 | g976256 | 31 | −16 | gb105pln | Rice mRNA stearyl-ACP desaturase, complete cds. | |
| 700342453H1 | g166818 | 38 | −30 | gb105pln | Arabidopsis thaliana protein kinase mRNA, complete cds. | |
| 700342364H1 | g2668737 | 33 | −48 | gb105pln | Zea mays translation initiation factor 5A (TIF5A) mRNA, complete cds. | |
| 700342902H1 | g170746 | 38 | −48 | gb105pln | Wheat histone H4 TH091 gene, complete cds. | |
| 700346126H1 | g1771160 | 26 | −6 | gb105eukp | SBT1; serine protease | |
| 700345663H1 | g1330395 | 17 | −10 | gb105eukp | C01F1.3 | |
| 700343176H1 | g1495273 | 21 | −4 | gb105eukp | sugar transporter | |
| 700346827H1 | g485744 | 25 | 6 | gb105allp | pyrophosphatase | |
| 700346207H1 | g1742893 | 9 | 6 | gb105allp | Sorbitol dehydrogenase (EC 1.1.1.14) (L-iditol 2-dehydrogenase). | |
| 700342378H1 | g168496 | 76 | −78 | gb105pln | Maize (Zea mays) histone H3 gene (H3C4), complete cds. | |
| 700343440H1 | g1184595 | 14 | 0 | gb105eukp | T11G6.2 | |
| 700344090H1 | g2632253 | 76 | −70 | gb105pln | S. bicolor mRNA for putative protein serine/threonine kinase, clone cSNFL2. | |
| 700380248H1 | g968901 | 54 | −44 | gb105pln | Rice mRNA for ribosomal protein S8, complete cds. | |
| 700343050H1 | g1323417 | 21 | −11 | gb105eukp | ORF YGR231c | |
| 700346238H1 | g303852 | 52 | −39 | gb105pln | Rice mRNA for ribosomal protein L3, complete cds. | |
| 700344411H1 | g2266661 | 16 | −35 | gb105pln | Hordeum vulgare mRNA for 14-3-3 protein (Hv1433c). | |
| 700346978H1 | g1129144 | 44 | −34 | gb105pln | M. indica (Manila) THMF5 mRNA for 3-ketoacyl-coA thiolase B. | |
| 700343507H1 | g474015 | 46 | −27 | gb105pln | Rice mRNA, partial homologous to calmodulin gene. | |
| 700344208H1 | g22280 | 10 | 6 | gb105eukp | GSTIII (aa 1-220) | |
| 700346790H1 | g1938235 | 25 | −14 | gb105pln | R. communis mRNA for acyl-CoA-binding protein. | |
| 700345505H1 | g2282583 | 93 | −84 | gb105pln | Zea mays elongation factor 1-alpha (EF1-A) mRNA, complete cds. | |
| 700342360H1 | g1620661 | 57 | −60 | gb105pln | Triticum aestivum 1,4-alpha-D-glucan 6-alpha-D-(1,4-alpha-D-glucanotransferase | |
| | | | | | mRNA, complete cds. | |
| 700343574H1 | g1575127 | 78 | −71 | gb105pln | Zea mays lumenal binding protein cBiPe2 mRNA, complete cds. | |
| 700345002H1 | g2739218 | 43 | −21 | gb105pln | Hordeum vulgare S28 ribosomal protein. | |
| 700346295H1 | g1749777 | 15 | −3 | gb105eukp | Minute(3)66D; ribosomal protein L14 | |
| 700345080H1 | g534915 | 23 | −24 | gb105pln | S. tuberosum (Desiree) ppa mRNA for soluble inorganic pyrophosphatase. | |
| 700380701H1 | g2342860 | 6 | 4 | gb105eukp | E4/E8 binding protein-1 | |
| 700379629H1 | g166548 | 14 | 13 | gb105pln | Avena sativa vacuolar H+-ATPase 16 kDa proteolipid subunit (vatp-P1) mRNA, | |
| | | | | | complete cds. | |
| 700342824H1 | g602605 | 36 | −58 | gb105pln | Zea mays tandem genes for alpha1-tubulin and alpha2-tubulin. | |
| 700380655H1 | g1658312 | 26 | −36 | gb105pln | O. sativa osr40g2 gene. | |
| 700380401H1 | g2695678 | 29 | −15 | gb105pln | Spinacia oleracea heat shock 70 protein (HSC70-11) mRNA, nuclear gene encoding | |
| | | | | | mitochondrial protein, complete cds. | |
| 700379869H1 | g968901 | 74 | −68 | gb105pln | Rice mRNA for ribosomal protein S8, complete cds. | |
| 700346080H1 | g158191 | 11 | −1 | gb105eukp | pum; pumilio protein | |
| 700345574H1 | g2160177 | 14 | 1 | gb105eukp | F21M12.26 | |
| 700342876H1 | g415316 | 57 | −43 | gb105pln | Rice mRNA for acidic ribosomal protein P0, complete cds. | |
| 700343903H1 | g536895 | 28 | −42 | gb105pln | Wheat mRNA for protein H2A, complete cds, clone wcH2A-10. | |
| 700379721H1 | g1707480 | 8 | 5 | gb105allp | CRM1 | |
| 700347063H1 | g1322489 | 38 | −24 | gb105eukp | STT3 | |
| 700380717H1 | g1262581 | 33 | 8 | gb105allp | cytochrome c oxidase subunit 1 | |
| 700380440H1 | g340201 | 18 | 5 | gb105allp | voltage-dependent anion channel | |
| 700347167H1 | g22151 | 56 | −35 | gb105pln | Z. mays (A188) mRNA for alpha-tubulin 4. | |
| 700346529H1 | g473602 | 46 | −36 | gb150pln | Zea mays W-22 histone H2A mRNA, complete cds. | |
| 700342477H1 | g2262141 | 7 | 5 | gb105eukp | T10P11.8; hypothetical protein | |
| 700345457H1 | g1905943 | 20 | −12 | gb105pln | Sorghum bicolor MADS box transcription factor SbMADS1 mRNA, complete cds. | |
| 700380658H1 | g2058313 | 29 | 8 | gb105eukp | CCR1; cinnamoyl-CoA reductase; EC 1.2.1.44 | |
| 700347195H1 | g587074 | 21 | 3 | gb105allp | putative IEF | |
| 700342934H1 | g1694975 | 25 | −6 | gb105pln | A. thaliana mRNA for HMG 1 protein. | |
| 700343250H1 | g2832689 | 19 | −13 | gb105pln | Arabidopsis thaliana DNA chromosome 4, BAC clone T9A21 (ESSAII project). | |
| 700343729H1 | g2244973 | 12 | −3 | gb105eukp | similarity to extensin class 1 protein | |
| 700380278H1 | g1200153 | 37 | −29 | gb105pln | S. tuberosum mRNA for starch synthase. | |
| 700347132H1 | g2282583 | 77 | −16 | gb105pln | Zea mays elongation factor 1-alpha (EF1-A) mRNA, complete cds. | |
| 700342967H1 | g20250 | 66 | −54 | gb105pln | Oryza sativa H3 hstone gene H3R-11. | |
| 700379668H1 | g5331 | 20 | −2 | gb105eukp | YCL033c, len:168 | |
| 700342580H1 | g950052 | 23 | −16 | gb105pln | H. vulgare mRNA for HMG1/2-like protein. | |
| 700342566H1 | g431265 | 14 | 14 | gb105pln | Arabidopsis thaliana ubiquitin conjugating enzyme (UBC4) gene, complete cds and | |
| | | | | | flanking regions. | |
| 700344058H1 | g176779 | 71 | −32 | gb105allp | gamma-2 globin | |
| 700345281H1 | g600768 | 41 | −33 | gb105pln | Oryza sativa cyclophilin 2 (Cyp2) mRNA, complete cds. | |
| 700342772H1 | g1183936 | 13 | −6 | gb105pln | P. sativum 5S rRNA gene. | |
| 700281924H2 | g1100222 | 42 | −34 | gb105pln | Pinus sylvestris chloroplast NAD+-dependent glyceraldehyde-3-phosphate dehydrogenase | |
| | | | | | mRNA, complete cds. | |
| 700342278H1 | g1335578 | 4 | 6 | gb105allp | gag p15 protein | |
| 700343958H1 | g1000464 | 16 | −5 | gb105eukp | C27B7.1 | |
| 700281881H2 | g1617213 | 19 | 3 | gb105eukp | CP12 | |
| 700346275H1 | g1519252 | 15 | −4 | gb105pln | Oryza sativa GF14-d protein mRNA, complete cds. | |
| 700343660H1 | g1049327 | 51 | −53 | gb105pln | Bourreria succulenta 18S ribosomal RNA gene, partial sequence. | |
| 700346889H1 | g2407800 | 32 | −3 | gb105eukp | H2Av; histone H2A.F/Z | |
| 700346694H1 | g1706958 | 86 | 1 | gb105eukp | celA2; cellulose synthase | |
| 700343221H1 | g1236272 | 24 | −14 | gb105eukp | putative ribosomal protein L26, most similar to pir: S51347 (68.6% identity in 121 aa | |
| | | | | | overlap) | |
| 700344621H1 | g4392 | 22 | −7 | gb105eukp | RPL37A; ribosomal protein L37a | |
| 700346518H1 | g1498385 | 54 | −62 | gb105pln | Zea mays actin (Maz87) gene, partial cds. | |
| 700343057H1 | g2618723 | 35 | −12 | gb105eukp | IAA17; IAA17 | |
| 700344630H1 | g2352494 | 7 | 5 | gb105eukp | TIR1; transport inhibitor response 1 | |
| 700380515H1 | g2570839 | 5 | 7 | gb105allp | synaptojanin 2 | |
| 700379656H1 | g2267592 | 38 | −34 | gb105pln | Oryza sativa glycine-rich RNA-binding protein mRNA, complete cds. | |
| 700343803H1 | g2244747 | 17 | −2 | gb105pln | Arabidopsis thaliana DNA chromosome 4, ESSA I contig fragment No. 0. | |
| 700342653H1 | g567106 | 15 | 4 | gb105allp | RNA binding protein | |
| 700281911H1 | g22275 | 18 | 16 | gb105pln | Maize mRNA for ferritin (clone FM1). | |
| 700345410H1 | g2262143 | 49 | −22 | gb105eukp | T10P11.10; putative serine/threonine protein kinase/T10P11.10 | |
| 700346440H1 | g310932 | 36 | −42 | gb105pln | Nicotiana tabacum ribosomal protein L17 mRNA, complete cds. | |
| 700345663H1 | g1072251 | 24 | −8 | gb105eukp | F53B1.4 | |
| 700344923H1 | g854625 | 42 | −46 | gb105pln | T. aestivum mRNA for peptidylprolyl cis-trans isomerase, FK506. | |
| 700346975H1 | g500638 | 7 | 7 | gb105allp | hnRNP protein A2 | |
| 700343359H1 | g2464924 | 18 | 2 | gb105eukp | hypothetical protein | |
| 700346959H1 | g1848280 | 20 | −15 | gb105pln | Sorghum bicolor membrane intrinsic protein (Mip1) mRNA, partial cds. | |
| 700343573H1 | g1107562 | 32 | −16 | gb105eukp | RpS13; ribosomal protein S13 | |
| 700344603H1 | g285965 | 80 | −42 | gb105allp | The sequence from bp313 to bp1374 is almost identical to human farnesyl pyrophosphate | |
| | | | | | synthetase mRNA. | |
| 700345132H1 | g1498052 | 73 | −21 | gb105pln | Zea mays ribosomal protein S8 mRNA, complete cds. | |
| 700343667H1 | g218082 | 19 | −19 | gb105pln | Rice mRNA for initiation factor eIF-4D (225 gene), partial sequence. | |
| 700344031H1 | g1523914 | 58 | 5 | gb105allp | F25H2.9 | |
| 700380164H1 | g602252 | 45 | −41 | gb105pln | Zea mays enolase (eno2) mRNA, complete cds. | |
| 700379608H1 | g886739 | 46 | −43 | gb105pln | Z. mays histone H4 gene. | |
| 700345454H1 | g35597 | 10 | 6 | gb105allp | PR 264 | |
| 700344241H1 | g398848 | 36 | −66 | gb105pln | Z. mays mRNA for beta 5 tubulin. | |
| 700343159H1 | g2781345 | 38 | −10 | gb105eukp | F24O1.2 | |
| 700343892H1 | g168502 | 50 | −38 | gb105pln | Maize (Zea mays) histone H4 gene (H4C7), complete cds. | |
| 700347095H1 | g287911 | 8 | 8 | gb105allp | CDC39 | |
| 700342692H1 | g1149570 | 48 | −36 | gb105pln | A. thaliana mRNA for mitochondrial elongation factor Tu. | |
| 700344325H1 | g42219 | 9 | 5 | gb105allp | P35 gene product (AA 1-314) | |
| 700344182H1 | g1143426 | 45 | −35 | gb105pln | C. sativus mRNA for heat shock protein 70 (hsp70). | |
| 700346213H1 | g2145472 | 31 | −26 | gb105pln | S. tuberosum mRNA for aconitase/aconitate hydratase. | |
| 700343041H1 | g1778820 | 40 | −40 | gb105pln | Oryza sativa S-adenosyl-L-methionine synthetase (pOS-SAMS2) mRNA, complete cds. | |
| 700379989H1 | g535588 | 15 | 1 | gb105eukp | atj; molecular chaperone | |
| 700345707H1 | g1556445 | 33 | −21 | gb105pln | Hordeum vulgare alpha tubulin (tubA) mRNA, complete cds. | |
| 700346114H1 | g312253 | 27 | −10 | gb105allp | calpactin I heavy chain | |
| 700345701H1 | g398605 | 11 | 1 | gb105pln | A. thaliana gene for elongation factor 1 beta. | |
| 700344661H1 | g1546918 | 90 | −41 | gb105pln | Z. mays mRNA for translation initiation factor 5A. | |
| 700343820H1 | g581023 | 8 | 5 | gb105allp | elongation factor 1-alpha | |
| 700380408H1 | g514394 | 11 | −1 | gb105pln | Catharanthus roseus cyc07 gene, exon1-7, complete cds. | |
| 700346953H1 | g557681 | 59 | −60 | gb105pln | Z. mays mRNA for ribosomal protein S28. | |
| 700346863H1 | g2764574 | 24 | −9 | gb105eukp | pore protein of 24 kD (OEP24) | |
| 700346708H1 | g22328 | 100 | −96 | gb105pln | Maize mRNA for a high mobility group protein. | |
| 700343857H1 | g2462742 | 18 | −12 | gb105eukp | F8A5.25 | |
| 700344979H1 | g710550 | 11 | 4 | gb105allp | ankyrin 3 | |
| 700343320H1 | g1230697 | 27 | −5 | gb105eukp | YPR094W; Ypr094wp | |
| 700342992H1 | g20163 | 41 | −23 | gb105pln | O. sativa Rr15 mRNA for 5S ribosomal RNA. | |
| 700343280H1 | g216516 | 11 | −1 | gb105allp | adenylate kinase | |
| 700342330H1 | g2407279 | 13 | 2 | gb105allp | aldolase | |
| 700344461H1 | g1632830 | 23 | 7 | gb105pln | R. communis mRNA (unknown). | |
| 700346249H1 | g1155217 | 11 | 3 | gb105eukp | Pti1; sereine/threonine protein kinase; Pto kinase interactor 1 | |
| 700345807H1 | g182518 | 28 | 4 | gb105allp | ferritin light subunit | |
| 700343490H1 | g1906827 | 26 | 3 | gb105pln | A. thaliana hsp81.4 gene. | |
| 700346917H1 | g2760323 | 36 | −8 | gb105eukp | F1N21.8 | |
| 700343439H1 | g170746 | 72 | −57 | gb105pln | Wheat histone H4 TH091 gene, complete cds. | |
| 700347257H1 | g1519248 | 50 | −42 | gb105pln | Oryza sativa GF14-b protein mRNA, complete cds. | |
| 700345910H1 | g2245126 | 12 | 14 | gb105pln | Arabidopsis thaliana DNA chromosome 4, ESSA I contig fragment No. 9. | |
| 700379656H1 | g2668741 | 87 | −79 | gb105pln | Zea mays glycine-rich RNA binding protein (GRP) mRNA, complete cds. | |
| 700347040H1 | g1322613 | 31 | −14 | gb105eukp | ORF YGL087c | |
| 700343162H1 | g460041 | 27 | −11 | gb105pln | Yeast (Saccharomyces cerevisiae) DBF3 gene, complete cds. | |
| 700380088H1 | g4352 | 29 | −12 | gb105pln | Yeast (Saccharomyces cerevisiae) gene for RNA-helicase. | |
| 700342206H1 | g170783 | 22 | −16 | gb105pln | T. aestivum ubiquitin carrier protein mRNA. | |
| 700343811H1 | g804948 | 26 | −14 | gb105eukp | acyl-(acyl carrier protein) thioesterase; EC 3.1.2.14 | |
| 700379988H1 | g1408470 | 19 | 5 | gb105pln | Arabidopsis thaliana actin depolymerizing factor 1 (AtADF1) mRNA, complete cds. | |
| 700281876H2 | g506860 | 92 | −11 | gb105eukp | HRSec61 | |
| 700344603H1 | g182399 | 80 | −43 | gb105allp | farnesyl pyrophosphate synthetase (EC 2.5.1.1) | |
| 700346721H1 | g2656016 | 9 | 6 | gb105allp | cell-cycle regulatory protein | |
| 700342744H1 | g1743006 | 23 | −6 | gb105pln | C. paradoxa mRNA for ribosomal protein L13a. | |
| 700380443H1 | g520935 | 32 | −47 | gb105pln | H. vulgare mRNA for gamma-TIP-like protein. | |
| 700342512H1 | g556018 | 57 | −49 | gb105pln | Barley glutamate 1-semialdehyde aminotransferase (GSA) mRNA, complete cds. | |
| 700344959H1 | g1504022 | 71 | −24 | gb105allp | KIAA0220 | |
| 700345930H1 | g2583106 | 23 | −2 | gb105pln | Arabidopsis thaliana chromosome II BAC F4L23 genomic sequence, complete sequence. | |
| 700346119H1 | g2529386 | 32 | −61 | gb105pln | Zea mays triosephosphate isomerase 1 gene, exon 2-9 and complete cds. | |
| 700346010H1 | g158308 | 29 | −13 | gb105eukp | Dsub\Rp49 | |
| 700343593H1 | g52783 | 33 | −17 | gb105allp | 47 kd keratin | |
| 700343104H1 | g187622 | 21 | −8 | gb105allp | MHC class I antigen | |
| 700345413H1 | g2511530 | 75 | −66 | gb105pln | Eleusine indica alpha tubulin 1 (TUA1) mRNA, complete cds. | |
| 700344310H1 | g662342 | 22 | −7 | gb105eukp | MAP1; Map1p: methionine aminopeptidase | |
| 700380717H1 | g13006 | 33 | 8 | gb105allp | cytochrome oxidase I | |
| 700379954H1 | g2618602 | 20 | −21 | gb105pln | Arabidopsis thaliana genomic DNA, chromosome 5, P1 clone; MSJ1, complete | |
| | | | | | sequence. | |
| 700345641H1 | g22322 | 18 | 2 | gb105pln | Z. mays mRNA for H2B histone (clone cH2B214). | |
| 700344418H1 | g643074 | 21 | 0 | gb105eukp | putative 40S ribosomal protein s12 | |
| 700343947H1 | g2739433 | 10 | 3 | gb105allp | hematopoietic-specific IL-2 deubiquitinating enzyme | |
| 700345012H1 | g1001312 | 13 | 1 | gb105allp | hypothetical protein | |
| 700344748H1 | g170132 | 44 | −39 | gb105pln | Spinach chloroplast ribosomal protein L13 mRNA, complete cds. | |
| 700379711H1 | g168498 | 23 | −20 | gb105pln | Corn histone H4 (H4C13) gene, complete cds. | |
| 700343490H1 | g300082 | 34 | −5 | gb105pln | hsp82 = 82 kda heat shock protein [Zea mays, seedling, leaves, Genomic, 3468 nt]. | |
| 700343303H1 | g577824 | 41 | −45 | gb105pln | Z. mays gene for H2B histone (gH2B3). | |
| 700345031H1 | g915312 | 39 | −23 | gb105pln | Nicotiana glutinosa ribosomal protein L31 mRNA, complete cds. | |
| 700343061H1 | g2351378 | 19 | −4 | gb105allp | eIF3-p66 | |
| 700346735H1 | g2511530 | 58 | −59 | gb105pln | Eleusine indica alpha tubulin 1 (TUA1) mRNA, complete cds. | |
| 700342945H1 | g1695692 | 22 | 1 | gb105eukp | diminuto | |
| 700343962H1 | g218182 | 30 | −25 | gb105pln | Rice mRNA for oryzain beta (EC 3.4.22). | |
| 700380765H1 | g1743356 | 39 | −9 | gb105eukp | proteasome delta subunit | |
| 700345572H1 | g218241 | 18 | −32 | gb105pln | Rice mRNA for ribosomal protein L3 (T82 gene), partial sequence. | |
| 700342273H1 | g13014 | 7 | 7 | gb105allp | URF 5 (NADH dehydrogenase subunit) | |
| 700343622H1 | g1296954 | 24 | −5 | gb105pln | O. sativa mRNA for novel protein, osr40c1. | |
| 700380255H1 | g473976 | 54 | −47 | gb105pln | Rice mRNA, partial homologous to elongation factor 1-alpha gene. | |
| 700380003H1 | g192924 | 49 | −27 | gb105allp | cytochrome c oxidase subunit Vb | |
| 700343107H1 | g1213441 | 38 | −17 | gb105pln | Arabidopsis thaliana isopentenyl pyrophosphate isomerase (Ipi1) mRNA, partial cds. | |
| 700344219H1 | g2827141 | 15 | 3 | gb105allp | cellulose synthase catalystic subunit | |
| 700345312H1 | g2462749 | 17 | −7 | gb105eukp | F8A5.31; Putative Serine/Threonine protein kinase | |
| 700347329H1 | g1653702 | 23 | 6 | gb105allp | dihydrolipoamide acetyltransferase component (E2) of pyruvate dehydrogenase complex | |
| 700346289H1 | g2414593 | 10 | 7 | gb105eukp | SPAC1A6.10; molybdopterin biosynthesis | |
| 700345568H1 | g2586332 | 20 | 4 | gb105pln | Lycopersicon esculentum importin alpha (LeKAP alpha) mRNA, partial cds. | |
| 700344426H1 | g182518 | 27 | −6 | gb105allp | ferritin light subunit | |
| 700379967H1 | g473604 | 51 | −44 | gb105pln | Zea mays W-22 histone H2B mRNA, complete cds. | |
| 700346948H1 | g474000 | 42 | −47 | gb105pln | Rice mRNA, partial homologous to ribosomal protein L37a gene. | |
| 700343655H1 | g2565339 | 38 | −20 | gb105pln | Lupinus luteus ribosomal protein S14 (rps14) mRNA, complete cds. | |
| 700344001H1 | g473986 | 40 | 11 | gb105pln | Rice mRNA, partial homologous to histone H2B gene. | |
| 700346640H1 | g471279 | 23 | −15 | gb105eukp | KRP-A | |
| 700345344H1 | g2558943 | 17 | −0 | gb105pln | Gossypium hirsutum histone 3 mRNA, complete cds. | |
| 700346812H1 | g433416 | 41 | −6 | gb105allp | TAXREB107 | |
| 700346275H1 | g1519248 | 11 | 9 | gb105pln | Oryza sativa GF14-b protein mRNA, complete cds. | |
| 700343515H1 | g2384759 | 51 | −47 | gb105pln | Oryza sativa GDP dissociation inhibitor protein OsGDI2 (OsGDI2) mRNA, | |
| | | | | | complete cds. | |
| 700346456H1 | g312569 | 15 | 16 | gb105pln | I. latifolius 26S rRNA (partial). | |
| 700345622H1 | g736308 | 17 | −4 | gb105eukp | unknown | |
| 700345233H1 | g2565339 | 28 | −33 | gb105pln | Lupinus luteus ribosomal protein S14 (rps14) mRNA, complete cds. | |
| 700346892H1 | g533251 | 54 | −62 | gb105pln | Zea mays (clone pSM8) sucrose synthase 2 (Sus1) gene, complete cds. | |
| 700344134H1 | g473985 | 36 | 1 | gb105pln | Rice mRNA, partial homologous to high mobility group protein gene. | |
| 700345118H1 | g434344 | 26 | −7 | gb105pln | A. thaliana (Columbia) mRNA for S18 ribosomal protein (641 bp). | |
| 700346886H1 | g2351061 | 18 | −6 | gb105pln | Arabidopsis thaliana genomic DNA, chromosome 5, P1 clone: MAF19. | |
| 700344696H1 | g2570125 | 28 | 2 | gb105allp | CPE-receptor | |
| 700345125H1 | g1707012 | 13 | 7 | gb105allp | tyrosyl-tRNA synthetase isolog | |
| 700346465H1 | g1931623 | 44 | −25 | gb105eukp | PCS1; proteasome cap subunit | |
| 700346778H1 | g790381 | 19 | −1 | gb105eukp | F13D12.4 | |
| 700342866H1 | g1498473 | 57 | −6 | gb105eukp | 60S ribosomal protein | |
| 700345685H1 | g2130944 | 13 | 3 | gb105allp | sulfate transporter | |
| 700343122H1 | g902583 | 73 | −57 | gb105pln | Zea mays clone MubG1 ubiquitin gene, complete cds. | |
| 700342629H1 | g2511573 | 37 | −21 | gb105pln | Arabidopsis thaliana mRNA for proteasome subunit prc3. | |
| 700342282H1 | g1122803 | 15 | 5 | gb105allp | F49C12.8 | |
| 700345009H1 | g1905943 | 33 | −20 | gb105pln | Sorghum bicolor MADS box transcription factor SbMADS1 mRNA, complete cds. | |
| 700342827H1 | g1008433 | 27 | −15 | gb105eukp | SPAC22F3.05c; unknown | |
| 700342423H1 | g300082 | 61 | −13 | gb105pln | hsp82 = 82 kda heat shock protein [Zea mays, seedling, leaves, Genomic, 3468 nt]. | |
| 700343864H1 | g577611 | 97 | −57 | gb105pln | Zea mays CRT1 gene for calcium-binding protein. | |
| 700379994H1 | g169818 | 49 | −60 | gb105pln | Rice 25S ribosomal RNA gene. | |
| 700345506H1 | g1050434 | 11 | 17 | gb105pln | b. napus mRNA for glyoxysomal malat edehydrogenase. | |
| 700342872H1 | g1763380 | 10 | 5 | gb105allp | zinc transporter | |
| 700347258H1 | g2293565 | 29 | −11 | gb105pln | Oryza sativa ADP-ribosylation factor 1 (Os-ARF1) mRNA, complete cds. | |
| 700343367H1 | g2662139 | 10 | 6 | gb105allp | KIAA0429 | |
| 700380130H1 | g2736290 | 11 | 7 | gb105allp | hMed7 | |
| 700346939H1 | g296173 | 27 | −13 | gb105pln | P. sativum ndk-p1 mRNA for nucleoside diphosphate kinase. | |
| 700345728H1 | g2708484 | 9 | 6 | gb105allp | IAA24 | |
| 700343506H1 | g968901 | 23 | −39 | gb105pln | Rice mRNA for ribosomal protein S8, complete cds. | |
| 700344724H1 | g2832300 | 16 | −5 | gb105eukp | CRakn; phosphorylates 3′-OH group of adenosine-5′-phosphosulfate; adenosine-5′- | |
| | | | | | phosphosulfate-kinase; EC 2.7.1.25 | |
| 700343921H1 | g1568481 | 21 | −3 | gb105pln | B. vulgaris mRNA for cdc2-related protein kinase. | |
| 700347153H1 | g237649 | 35 | −24 | gb105pln | enoyl-acyl carrier protein reductase [Brassica napus, mRNA, 1358 nt]. | |
| 700281817H2 | g2253314 | 8 | 1 | gb105allp | RecA | |
| 700344130H1 | g506533 | 24 | −3 | gb105pln | N. tabacum PKTL7 mRNA for protein kinase. | |
| 700343624H1 | g473604 | 37 | −22 | gb105pln | Zea mays W-22 histone H2B mRNA, complete cds. | |
| 700343374H1 | g606969 | 30 | −17 | gb105pln | Arabidopsis thaliana cytoplasmic ribosomal protein L18 mRNA, complete cds. | |
| 700343230H1 | g2828186 | 13 | 14 | gb105pln | Arabidopsis thaliana genomic DNA, chromosome 5, TAC clone: K18I23, complete | |
| | | | | | sequence. | |
| 700281848H2 | g313151 | 32 | 5 | gb105pln | A. thaliana ribosomal protein S15 mRNA, complete CDS. | |
| 700345755H1 | g1498385 | 77 | −72 | gb105pln | Zea mays actin (Maz87) gene, partial cds. | |
| 700342533H1 | g386765 | 65 | −8 | gb105allp | hba1 alpha globin | |
| 700342485H1 | g218088 | 40 | −30 | gb105pln | Rice mRNA for ribosomal protein 117 (249 gene), partial sequence. | |
| 700380642H1 | g16732 | 25 | −0 | gb105allp | NADH ubiquinone oxidoreductase subunit | |
| 700344425H1 | g2340104 | 26 | 1 | gb105allp | Very similar and perhaps idential to Hs-CUL-4B.; 80-100% similarity to partial sequence | |
| | | | | | U58091 (PID:g1381150). | |
| 700347080H1 | g587074 | 28 | 8 | gb105allp | putative IEF | |
| 700342593H1 | g166868 | 33 | −24 | gb105pln | Arabidopsis thaliana ribosomal protein S11 (RPS11-beta) mRNA, complete cds. | |
| 700344965H1 | g2181190 | 19 | −2 | gb105eukp | BRLK; serine/threonine kinase | |
| 700346709H1 | g1419389 | 39 | −34 | gb105pln | A. thaliana mRNA for thylakoid-bound ascorbate peroxidase. | |
| 700343712H1 | g2330820 | 18 | 3 | gb105eukp | SPAC4F8.03; hypothetical protein | |
| 700347153H1 | g2653284 | 54 | −44 | gb105pln | Oryza sativa mRNA for enoyl-ACP reductase. | |
| 700346816H1 | g1651729 | 8 | 5 | gb105allp | erthyrocyte band 7 integral membrane protein, protein 7.2B, stomatin | |
| 700346587H1 | g1617267 | 28 | −11 | gb105pln | B. napus mRNA for acyl CoA synthetase. | |
| 700344516H1 | g1272628 | 27 | −9 | gb105eukp | F44G4.1 | |
| 700344757H1 | g31924 | 34 | −17 | gb105allp | glutathione S-transferase (AA 1-218) | |
| 700344960H1 | g21794 | 79 | −70 | gb105pln | Wheat histone H4 gene. | |
| 700346779H1 | g2388950 | 13 | 1 | gb105eukp | SPAC27E2.10c; activator 1 protein | |
| 700380261H1 | g633940 | 29 | −16 | gb105eukp | PTR2-B; peptide transporter; transport protein | |
| 700343309H1 | g386765 | 60 | −27 | gb105allp | hba1 alpha globin | |
| 700380603H1 | g762850 | 7 | 6 | gb105eukp | wos2; p21 protein | |
| 700346421H1 | g22324 | 15 | −7 | gb105pln | Z. mays mRNA for H2B histone (clone cH2B221). | |
| 700379661H1 | g168557 | 64 | −27 | gb105pln | Zea mays putative GTP-binding protein homolog mRNA, partial cds. | |
| 700343942H1 | g21794 | 73 | −59 | gb105pln | Wheat histone H4 gene. | |
| 700346445H1 | g21134 | 68 | −53 | gb105pln | Sinapis alba gene for 25S rRNA. | |
| 700281932H2 | g2369713 | 17 | −5 | gb105pln | Beta vulgaris cDNA for elongation factor 2. | |
| 700346554H1 | g2828180 | 12 | 15 | gb105pln | Arabidopsis thaliana genomic DNA, chromosome 5, P1 clone: MDK4, complete | |
| | | | | | sequence. | |
| 700346277H1 | g886737 | 70 | −20 | gb105pln | Z. mays histone H3 gene. | |
| 700344173H1 | g1185553 | 36 | −61 | gb105pln | Zea mays glyceraldehyde-3-phosphate dehydrogenase (gpc2) gene, complete cds. | |
| 700347076H1 | g1322489 | 17 | −8 | gb105eukp | STT3 | |
| 700346894H1 | g2749918 | 15 | 17 | gb105pln | Arabidopsis thaliana chromosome I BAC F3I6 genomic sequence, complete sequence. | |
| 700346406H1 | g19539 | 9 | −1 | gb105eukp | ribosomal protein YL16 | |
| 700345991H1 | g168549 | 100 | −49 | gb105pln | Zea mays putative anthranilate synthase component II homolog mRNA, partical cds. | |
| 700345824H1 | g599722 | 45 | −33 | gb105pln | C. melo mRNA for aconitase (UNI-ZAPxR). | |
| 700342370H1 | g168505 | 58 | −49 | gb105pln | Zea mays histone H3 gene, complete cds. | |
| 700343386H1 | g606043 | 13 | 6 | gb105allp | ORF_o328 | |
| 700344627H1 | g736271 | 55 | −48 | gb105pln | O. sativa hsp70 gene for heat shock protein 70. | |
| 700344667H1 | g38225 | 75 | 3 | gb105allp | alpha2-globin | |
| 700345450H1 | g2145472 | 29 | −0 | gb105pln | S. tuberosum mRNA for aconitase/aconitate hydratase. | |
| 700343624H1 | g21800 | 34 | −37 | gb105pln | T. aestivum L mRNA for hsitone H2B. | |
| 700343841H1 | g2394228 | 26 | −1 | gb105pln | Arabidopsis thaliana WD-40 repeat protein (MSI1) mRNA, complete cds. | |
| 700345326H1 | g452165 | 29 | 1 | gb105eukp | monodehydroascorbate reductase; EC 1.6.5.4 | |
| 700379989H1 | g2370312 | 16 | −1 | gb105eukp | msj1; molecular chaperone; DnaJ-like protein | |
| 700346393H1 | g2564051 | 37 | −20 | gb105pln | Arabidopsis thaliana genomic DNA, chromosome 5, P1 clone: MWD9, complete | |
| | | | | | sequence. | |
| 700344137H1 | g2842490 | 11 | 7 | gb105eukp | F21O9.160; heat-shock protein | |
| 700347165H1 | g2903 | 23 | −6 | gb105eukp | inorganic pyrophosphatase (AA 1-287) | |
| 700379974H1 | g439585 | 51 | −32 | gb105pln | Hordeum vulgare calreticulin (CRH1) mRNA, partial cds. | |
| 700346535H1 | g2266661 | 39 | −20 | gb105pln | Hordeum vulgare mRNA for 14-3-3 protein (Hv1433c). | |
| 700343215H1 | g2662342 | 60 | −43 | gb105pln | Oryza sativa mRNA for EF-1 alpha, complete cds. | |
| 700380717H1 | g506829 | 33 | 8 | gb105allp | cytochrome oxidase subunit 1 | |
| 700346083H1 | g1906829 | 18 | 8 | gb105pln | A. thaliana hsp88.1 gene. | |
| 700379966H1 | g2656029 | 12 | 16 | gb105pln | Arabidopsis thaliana genomic DNA, chromosome 5, P1 clone: MQB2. | |
| 700347052H1 | g1841467 | 21 | −3 | gb105pln | N. tabacum mRNA for tyrosyl-tRNA synthetase. | |
| 700345191H1 | g1143389 | 21 | 3 | gb105pln | A. thaliana mRNA for 3-hydroxy-3-methylglutaryl-CoA synthase. | |
| 700345991H1 | g403433 | 35 | −22 | gb105pln | Arabidopsis thaliana anthranilate synthase beta subunit 9 (ASB1) mRNA, complete cds. | |
| 700342514H1 | g349761 | 16 | 6 | gb105allp | RNA binding protein | |
| 700343251H1 | g483546 | 15 | 10 | gb105pln | R. communis gene for pyrophosphate-dependent phosphofructokinase alpha subunit. | |
| 700344416H1 | g1302004 | 7 | 7 | gb105allp | ORF YNL096c | |
| 700380285H1 | g167687 | 20 | −3 | gb105pln | Dictyostelium discoideum clathrin heavy chain (chcA) mRNA, complete cds. | |
| 700347163H1 | g1397320 | 16 | 7 | gb105allp | Similar to histone H2A variant. | |
| 700345545H1 | g2624199 | 41 | −34 | gb105pln | M. acuminata mRNA; clone pBAN UU93. | |
| 700343313H1 | g1532212 | 20 | 6 | gb105eukp | cypA; cyclophilin A | |
| 700344644H1 | g218130 | 32 | −16 | gb105pln | Rice mRNA for Ribosomal protein S15. | |
| 700346540H1 | g2104526 | 48 | −23 | gb105eukp | T10M13.3; T10M13.3 | |
| 700343287H1 | g1905943 | 58 | −39 | gb105pln | Sorghum bicolor MADS box transcription factor SbMADS1 mRNA, complete cds. | |
| 700345911H1 | g436029 | 27 | −14 | gb105pln | Nicotiana tabacum (TSC40-3) 60S ribosomal protein L34 mRNA, complete cds. | |
| 700281842H2 | g2262111 | 10 | 1 | gb105allp | ribitol dehydrogenase isolog | |
| 700344301H1 | g1707656 | 12 | 14 | gb105pln | P. sativum mRNA for DnaJ-like protein. | |
| 700343707H1 | g168494 | 16 | −17 | gb105pln | Maize (Zea mays) histone H3 gene (H3C2), complete cds. | |
| 700346435H1 | g2462756 | 7 | 0 | gb105eukp | F8A5.15; putative receptor kinase | |
| 700345521H1 | g3264 | 17 | 2 | gb105pln | P. tannophilus GAL 10 gene for UDP-galactose-4-epimerase. | |
| 700343350H1 | g577818 | 78 | −84 | gb105pln | Z. mays gene for H2B histone (gH2B4). | |
| 700379960H1 | g473986 | 23 | −30 | gb105pln | Rice mRNA, partial homologous to histone H2B gene. | |
| 700346667H1 | g2275202 | 12 | 5 | gb105eukp | T08I13.8; acyl-CoA synthetase isolog | |
| 700346456H1 | g312598 | 16 | 16 | gb105pln | M. ternifolia 26S rRNA (partial). | |
| 700342876H1 | g1196896 | 45 | −31 | gb105pln | Glycine max acidic ribosomal protein P0 mRNA, complete cds. | |
| 700380613H1 | g22322 | 16 | −25 | gb105pln | Z. mays mRNA for H2B histone (clone cH2B214). | |
| 700347270H1 | g577736 | 36 | −23 | gb105pln | A. thaliana PRL2 mRNA. | |
| 700346812H1 | g695638 | 38 | −13 | gb105allp | M-TAXREB107 | |
| 700343339H1 | g1345430 | 17 | 5 | gb105allp | perchrolic acid soluble protein | |
| 700343641H1 | g393399 | 10 | 17 | gb105pln | Z. mays Irma and En1 element DNA. | |
| 700345209H1 | g493707 | 61 | −56 | gb105pln | Ric emRNA for beta-tubulin, complete cds. | |
| 700380124H1 | g268 | 11 | 5 | gb105allp | cytochrome c oxidase subunit VIb (AA 1-86) | |
| 700347362H1 | g2760316 | 11 | 15 | gb105pln | The sequence of BAC F1N21 from Arabidopsis thaliana chromosome 1, complete | |
| | | | | | sequence. | |
| 700345728H1 | g2245378 | 14 | 2 | gb105allp | auxin response factor 1 | |
| 700343904H1 | g218130 | 32 | −26 | gb105pln | Rice mRNA for Ribosomal protein S15. | |
| 700344247H1 | g1498387 | 82 | −72 | gb105pln | Zea mays actin (Maz83) gene, partial cds. | |
| 700345356H1 | g2760322 | 14 | −3 | gb105eukp | F1N21.7 | |
| 700380469H1 | g14016 | 18 | −1 | gb105allp | cytochrome C oxidase II subunit | |
| 700347323H1 | g515865 | 13 | 2 | gb105allp | ribosomal protein L15 gene product | |
| 700346346H1 | g2104546 | 13 | 16 | gb105pln | Arabidopsis thaliana chromosome IV cosmid AGAA, complete sequence. | |
| 700342958H1 | g2642212 | 61 | −86 | gb105pln | Zea mays nitrate-induced NOI protein gene, complete cds. | |
| 700379637H1 | g1432082 | 26 | −5 | gb105pln | Arabidopsis thaliana Skp1p homolog mRNA, complete cds. | |
| 700345577H1 | g2739216 | 24 | 5 | gb105pln | Hordeum vulgare L41 ribosomal protein. | |
| 700346815H1 | g1834353 | 10 | 5 | gb105eukp | 194; hypothetical protein 194 | |
| 700342640H1 | g1197168 | 49 | −20 | gb105eukp | MHCemb; embryonic muscle myosin heavy chain | |
| 700343685H1 | g20311 | 45 | −54 | gb105pln | O. sativa R22 mRNA. | |
| 700346965H1 | g2244996 | 7 | 6 | gb105eukp | similarity to a membrane-associated salt-inducible protein | |
| 700344980H1 | g167107 | 23 | −15 | gb105pln | Hordeum vulgare vacuolar ATPase B subunit isoform mRNA, complete cds. | |
| 700346427H1 | g2213621 | 20 | −12 | gb105eukp | F21J9.26 | |
| 700345025H1 | g1065484 | 7 | 6 | gb105eukp | K02G10.6 | |
| 700346548H1 | g14029 | 13 | 4 | gb105eukp | rp15; ribosomal protein L5 | |
| 700344653H1 | g1322276 | 32 | 11 | gb105pln | Triticum aestivum histone H2A gene, complete cds. | |
| 700345018H1 | g2832242 | 36 | 16 | gb105pln | Zea mays 22-kDa alpha zein gene cluster, complete sequence. | |
| 700345821H1 | g415316 | 58 | −43 | gb105pln | Rice mRNA for acidic ribosomal protein P0, complete cds. | |
| 700345621H1 | g2827624 | 11 | 4 | gb105eukp | hypothetical protein | |
| 700344369H1 | g1160445 | 9 | 1 | gb105allp | ribosomal protein S7 | |
| 700380233H1 | g2656025 | 36 | −29 | gb105pln | Arabidopsis thaliana genomic DNA, chromosome 5, P1 clone: MCD7. | |
| 700346904H1 | g602252 | 100 | −85 | gb105pln | Zea mays enolase (eno2) mRNA, complete cds. | |
| 700346069H1 | g57720 | 40 | −9 | gb105allp | ribosomal protein S20 (AA 1-119) | |
| 700344387H1 | g157564 | 13 | −3 | gb105eukp | Aats-glupro; transfer RNA-Glu-Pro aminoacyl synthetase | |
| 700345323H1 | g644491 | 82 | −76 | gb105pln | Corn mRNA for elongation factor 1A. | |
| 700346305H1 | g625108 | 48 | −8 | gb105eukp | YLR388W; Ylr388wp | |
| 700281862H2 | g2655098 | 9 | 4 | gb105eukp | PTR; PTR | |
| 700344145H1 | g1399567 | 46 | −41 | gb105pln | Podophyllum peltatum nuclear 26S ribosomal RNA gene, partial sequence. | |
| 700344460H1 | g2696018 | 29 | 6 | gb105pln | Arabidopsis thaliana genomic DNA, chromosome 5, P1 clone: MXC9. | |
| 700380479H1 | g437878 | 14 | 8 | gb105allp | mrp S24 gene product | |
| 700343276H1 | g460041 | 28 | −14 | gb105pln | Yeast (Saccharomyces cerevisiae) DBF3 gene, complete cds. | |
| 700344422H1 | g2191128 | 34 | −18 | gb105eukp | A_IG002N01.3 | |
| 700345179H1 | g438279 | 20 | −2 | gb105allp | Ribosomal protein L7 | |
| 700344228H1 | g1322276 | 13 | 4 | gb105pln | Triticum aestivum histone H2A gene, complete cds. | |
| 700346824H1 | g2369765 | 20 | 2 | gb105pln | Citrus paradisi mRNA for hypothetical protein. | |
| 700345767H1 | g2708737 | 8 | 6 | gb105allp | putative nuclear protein | |
| 700343035H1 | g1301621 | 9 | 5 | gb105eukp | C08B6.7 | |
| 700343795H1 | g1276828 | 14 | −5 | gb105eukp | trpG; anthranilate synthase component II | |
| 700379846H1 | g1929057 | 16 | 7 | gb105eukp | longest ORF (1) | |
| 700342644H1 | g532126 | 9 | −3 | gb105eukp | eas; phosphorylate ethanolamine; ethanolamine kinase | |
| 700343836H1 | g2440191 | 17 | −10 | gb105eukp | hypothetical protein | |
| 700343240H1 | g214758 | 32 | −6 | gb105allp | ribosomal protein S22, 40S subunit | |
| 700342882H1 | g790969 | 47 | 2 | gb105pln | Rice mRNA for aldolase C-1, complete cds. | |
| 700379945H1 | g170775 | 82 | −70 | gb105pln | Wheat translation elongation factor 1 alpha-subunit (TEF1) mRNA, complete cds. | |
| 700347050H1 | g1553128 | 15 | 16 | gb105pln | Gossypium hirsutum ribosomal protein L44 isoform a (RL44), complete cds. | |
| 700346684H1 | g1483150 | 20 | 3 | gb105eukp | monodehydroascorbate reductase | |
| 700380663H1 | g1710150 | 21 | −3 | gb105pln | Arabidopsis thaliana proline iminopeptidase mRNA, complete cds. | |
| 700343316H1 | g296949 | 60 | −29 | gb105allp | glutaminyl-peptide cyclotransferase | |
| 700342872H1 | g1763376 | 10 | 5 | gb105allp | ZnT-3 | |
| 700379608H1 | g170746 | 46 | −44 | gb105pln | Wheat histone H4 TH091 gene, complete cds. | |
| 700346683H1 | g4442 | 22 | −4 | gb105pln | Yeast SEC23 gene for cytoplasmic transport protein. | |
| 700342961H1 | g902622 | 23 | −10 | gb105eukp | 40S ribosomal protein S12 | |
| 700345460H1 | g485273 | 40 | −17 | gb105eukp | CDC46; Cdc46p | |
| 700343672H1 | g2832242 | 34 | −9 | gb105pln | Zea mays 22- kDa alpha zein gene cluster, complete sequence. | |
| 700344177H1 | g2160756 | 6 | 5 | gb105allp | CLV1 receptor kinase | |
| 700344755H1 | g531055 | 45 | −63 | gb105pln | Wheat mRNA for protein H2B-6, complete cds. | |
| 700345273H1 | g463856 | 30 | −13 | gb105pln | Chlamydomonas reinhardtii 21gr ribosomal protein S14 (CRY1) gene, complete cds. | |
| 700344137H1 | g1931643 | 12 | 8 | gb105allp | DnaJ isolog | |
| 700380130H1 | g1420026 | 12 | 4 | gb105allp | ORF YOL135c | |
| 700344316H1 | g736308 | 14 | −1 | gb105eukp | unknown | |
| 700345389H1 | g454913 | 17 | −17 | gb105pln | A. porrum LDJ2 mRNA. | |
| 700347066H1 | g1839244 | 19 | 5 | gb105allp | EGF receptor like protein | |
| 700343233H1 | g1262999 | 21 | −9 | gb105eukp | ZK287.5 | |
| 700344448H1 | g416150 | 41 | −55 | gb105pln | Zea mays beta-8 tubulin (tub8) mRNA, complete cds. | |
| 700345155H1 | g520501 | 20 | 7 | gb105eukp | NADPH; thioredoxin reductase | |
| 700345467H1 | g2264321 | 29 | −15 | gb105pln | Arabidopsis thaliana genomic DNA, chromosome 5, P1 clone: MXM12, complete | |
| | | | | | sequence. | |
| 700344637H1 | g2081627 | 27 | −10 | gb105eukp | alpha-glucosidase precoursor; EC 3.2.1.20 | |
| 700345910H1 | g2245131 | 13 | −2 | gb105eukp | hypothetical protein | |
| 700281822H2 | g1143427 | 72 | 5 | gb105eukp | hsp70; heat shock protein 70 | |
| 700343115H1 | g1117927 | 23 | 1 | gb105eukp | ADE1; bifunctional peptide with both glycinamide ribotide synthetase and amino- | |
| | | | | | imidazole ribotide synthetase activites; Ade1p | |
| 700346443H1 | g2668747 | 31 | −14 | gb105pln | Zea mays ribosomal protein L17 (rp117) mRNA, complete cds. | |
| 700345573H1 | g1256607 | 15 | 7 | gb105pln | Glyzine max G protein beta subunit mRNA, complete cds. | |
| 700344138H1 | g2731767 | 11 | 7 | gb105allp | endonuclease/reverse transcriptase | |
| 700345457H1 | g939784 | 20 | −15 | gb105pln | Zea mays MADS-box protein (ZAP1) mRNA, complete cds. | |
| 700344792H1 | g2182652 | 13 | −1 | gb105allp | Y4uB | |
| 700380469H1 | g2245560 | 18 | −1 | gb105allp | cytochrome c oxidase subunit II | |
| 700344632H1 | g643076 | 19 | −3 | gb105eukp | annexin | |
| 700342403H1 | g1213334 | 9 | 4 | gb105allp | OrfX; hypothetical 22.5 KD protein downstream of tyhpe IV prepilin leader peptidase | |
| | | | | | gene; Method: conceptual translation supplied by author | |
| 700343440H1 | g1184596 | 14 | 1 | gb105eukp | T11G6.3 | |
| 700344341H1 | g2258468 | 46 | −34 | gb105pln | Oryza sativa replication protein A1 (Os-RPA1) mRNA, complete cds. | |
| 700345357H1 | g967984 | 44 | −29 | gb105pln | Oryza sativa (clone rma630) ribosomal protein-linked ubiquitin mRNA, complete cds. | |
| 700345391H1 | g218152 | 34 | −50 | gb105pln | Rice serine cbp3 gene for carboxypeptidase III. | |
| 700342686H1 | g2459420 | 40 | −5 | gb105eukp | F4P9.14; putative ribosomal protein L17 | |
| 700344784H1 | g1732367 | 15 | 5 | gb105allp | proteinase TMP | |
| 700346096H1 | g168500 | 54 | 3 | gb105pln | Maize (Zea mays) histone H4 gene (H4C14), complete cds. | |
| 700380614H1 | g2662344 | 79 | −77 | gb105pln | Oryza sativa mRNA for EF-1 alpha, complete cds. | |
| 700342423H1 | g1906827 | 56 | −10 | gb105pln | A. thaliana hsp81.4 gene. | |
| 700343651H1 | g168543 | 100 | −36 | gb105pln | Zea mays putative ribosomal protein S8 mRNA, partial cds. | |
| 700346165H1 | g13339 | 62 | −2 | gb105pln | P. hybrida mitochondrial rps19 gene for ribosomal protein S19. | |
| 700346459H1 | g168500 | 54 | −52 | gb105pln | Maize (Zea mays) histone H4 gene (H4C14), complete cds. | |
| 700344251H1 | g2252844 | 27 | −9 | gb105eukp | A_IG005I10.21 | |
| 700345053H1 | g2114264 | 10 | 2 | gb105allp | topoisomerase-III | |
| 700345866H1 | g2829918 | 11 | 8 | gb105eukp | F22K20.1 | |
| 700343941H1 | g2351066 | 25 | −11 | gb105pln | Arabidopsis thaliana genomic DNA, chromosome 5, P1 clone: MOP9. | |
| 700380290H1 | g644491 | 59 | −48 | gb105pln | Corn mRNA for elongation factor 1A. | |
| 700342732H1 | g556685 | 74 | −72 | gb105pln | Z. mays mRNA for ADP-ribosylation factor. | |
| 700380266H1 | g1877523 | 17 | 4 | gb105pln | Arabidopsis thaliana BAC T7I23, complete sequence. | |
| 700343623H1 | g2511745 | 19 | −5 | gb105eukp | dre4; probable transcriptional regulator dre4 | |
| 700379960H1 | g22324 | 36 | −39 | gb105pln | Z. mays mRNA for H2B histone (clone cH2B221). | |
| 700379641H1 | g551259 | 28 | −5 | gb105eukp | Ntbfruc1; beta-fructofuranosidase; EC 3.2.1.26 | |
| 700343438H1 | g295925 | 21 | −8 | gb105eukp | RPG19; ribosomal protein | |
| 700343407H1 | g2688822 | 32 | −13 | gb105eukp | pyrophosphate-dependent phosphofructo-1-kinase | |
| 700344108H1 | g1183936 | 14 | −1 | gb105pln | P. sativum 5S rRNA gene. | |
| 700380164H1 | g19280 | 47 | −42 | gb105pln | L. esculentum mRNA for enolase. | |
| 700345772H1 | g1276967 | 23 | −16 | gb105eukp | putative ribosomal protein | |
| 700343561H1 | g479146 | 14 | 5 | gb105eukp | putative ATP synthase subunit | |
| 700343332H1 | g2114027 | 18 | −2 | gb105eukp | ntf-2; putative nuclear transport factor 2 | |
| 700342345H1 | g433040 | 15 | −14 | gb105pln | Zea mays W-22 clone PREM-1A retroelement PREM-1, partial sequence. | |
| 700343820H1 | g47488 | 8 | 6 | gb105allp | elongation factor Tu3 | |
| 700344871H1 | g2213932 | 9 | 7 | gb105allp | 26S proteasome regulatory subunit | |
| 700343602H1 | g312178 | 40 | −10 | gb105pln | Z. mays GapC2 gene. | |
| 700379653H1 | g1929057 | 46 | −10 | gb105eukp | longest ORF (1) | |
| 700345729H1 | g544506 | 14 | −0 | gb105eukp | SIK1; Sik1p | |
| 700346995H1 | g2599091 | 28 | −7 | gb105pln | Arabidopsis thaliana WD-40 repeat protein MSI4 (MSI4) mRNA, complete cds. | |
| 700345415H1 | g2459406 | 44 | −40 | gb105pln | Arabidopsis thaliana chromosome II BAC F4P9 genomic sequence, complete sequence. | |
| 700347290H1 | g1020414 | 65 | −54 | gb105pln | Oryza sativa mRNA for phospholipase D, complete cds. | |
| 700345848H1 | g531055 | 47 | −30 | gb105pln | Wheat mRNA for protein H2B-6, complete cds. | |
| 700345809H1 | g21794 | 13 | 16 | gb105pln | Wheat histone H4 gene. | |
| 700346464H1 | g1480670 | 12 | 5 | gb105allp | delta 1-pyrroline-5-carboxylate synthetase | |
| 700345695H1 | g2662340 | 67 | −62 | gb105pln | Oryza sativa mRNA for EF-1 alpha, complete cds. | |
| 700343673H1 | g169776 | 16 | −2 | gb105pln | Oryza sativa beta-amylase gene, complete cds. | |
| 700347192H1 | g1938575 | 39 | −18 | gb105eukp | B0025.3 | |
| 700343110H1 | g2832667 | 30 | −5 | gb105pln | Arabidopsis thaliana DNA chromosome 4, BAC clone T10I14 (ESSAII project). | |
| 700343020H1 | g467038 | 11 | −5 | gb105allp | B2168_F3_130 | |
| 700342447H1 | g1167827 | 34 | −25 | gb105pln | c. reinhardtii mRNA for ribosomal protein L12. | |
| 700346839H1 | g2582641 | 17 | 6 | gb105allp | splicing factor | |
| 700346727H1 | g168500 | 76 | −68 | gb105pln | Maize (Zea mays) histone H4 gene (H4C14), complete cds. | |
| 700346795H1 | g416256 | 17 | −12 | gb105pln | Rice mRNA for H+-ATPase, partial sequence. | |
| 700345587H1 | g1890576 | 57 | −45 | gb105pln | H. vulgare mRNA for xyloglucan endotransglycosylase-like protein (XEB). | |
| 700379739H1 | g556217 | 9 | 7 | gb105allp | spliceosomal protein | |
| 700344010H1 | g22324 | 22 | −18 | gb105pln | Z. mays mRNA for H2B histone (clone cH2B221). | |
| 700342543H1 | g17863 | 13 | 3 | gb105allp | r-protein BnS15a | |
| 700379831H1 | g20321 | 58 | −48 | gb105pln | Oryza sativa RAc1 mRNA for actin. | |
| 700343326H1 | g2408068 | 11 | 6 | gb105eukp | SPAC2F3.16; hypothetical protein | |
| 700342651H1 | g1495270 | 27 | −0 | gb105pln | A. thaliana mRNA for RNA helicase. | |
| 700379988H1 | g929917 | 24 | −2 | gb105pln | Z. mays ABP1 mRNA for actin-depolymerizing factor. | |
| 700343051H1 | g2842494 | 58 | −37 | gb105eukp | F21O9.200; prohibitin-like protein | |
| 700281979H2 | g2696018 | 12 | 16 | gb105pln | Arabidopsis thaliana genomic DNA, chromosome 5, P1 clone: MXC9. | |
| 700281843H2 | g2821961 | 12 | 6 | gb105eukp | spermidine synthase; EC 2.5.1.16 | |
| 700342264H1 | g170783 | 18 | −12 | gb105pln | T. aestivum ubiquitin carrier protein mRNA. | |
| 700343001H1 | g1729537 | 37 | −16 | gb105eukp | F10C2.4 | |
| 700344976H1 | g2407799 | 25 | −6 | gb105pln | Arabidopsis thaliana mRNA for hsitone H2A.F/Z. | |
| 700346511H1 | g2655295 | 9 | 6 | gb105allp | germin-like protein 6 | |
| 700344031H1 | g296740 | 58 | 5 | gb105allp | macropain subunit zeta | |
| 700379996H1 | g2809537 | 14 | 5 | gb105allp | beta prime coatomer protein | |
| 700343587H1 | g288063 | 21 | −11 | gb105eukp | ketol-acid reductoisomerase; EC 1.1.1.86 | |
| 700342672H1 | g473602 | 94 | −80 | gb105pln | Zea mays W-22 histone H2A mRNA, complete cds. | |
| 700345557H1 | g2645166 | 18 | −1 | gb105pln | Oryza sativa mRNA, similar to ubiquitin conjugating enzyme. | |
| 700343031H1 | g168505 | 66 | −55 | gb105pln | Zea mays histone H3 gene, complete cds. | |
| 700347383H1 | g1388084 | 21 | −2 | gb105eukp | TRX5; thioredoxin h | |
| 700344993H1 | g16432 | 20 | 4 | gb105pln | A. thaliana mRNA encoding protein phosphastase 1A. | |
| 700346715H1 | g1408470 | 13 | 8 | gb105pln | Arabidopsis thaliana actin depolymerizing factor 1 (AtADF1) mRNA, complete cds. | |
| 700343374H1 | g2529657 | 15 | −2 | gb105pln | Arabidopsis thaliana chromosome II BAC T30B22 genomic sequence, complete | |
| | | | | | sequence. | |
| 700342952H1 | g2213449 | 16 | 1 | gb105allp | vitamin D inducible protein | |
| 700344132H1 | g1778569 | 8 | 7 | gb105allp | YaaF homolog | |
| 700343685H1 | g468055 | 56 | −66 | gb105pln | Zea mays B73 QM protein mRNA, complete cds. | |
| 700344747H1 | g2274801 | 18 | −4 | gb105eukp | ribosomal protein L4 | |
| 700345376H1 | g2352931 | 22 | 0 | gb105eukp | cCM2; cytosolic chorismate mutase 2 | |
| 700345620H1 | g1592120 | 6 | 6 | gb105allp | 2-phosphoglycerate kinase (2pgk) | |
| 700345245H1 | g2088722 | 21 | −4 | gb105eukp | F53G12.10 | |
| 700345772H1 | g1669623 | 15 | 1 | gb105eukp | ribosomal protein L39 | |
| 700345556H1 | g2811284 | 6 | 7 | gb105allp | ribosomal protein S7 | |
| 700342360H1 | g1885343 | 57 | −60 | gb105pln | T. aestivum mRNA for starch branching enzyme II. | |
| 700345348H1 | g1184775 | 65 | −45 | gb105pln | Zea mays glyceraldehyde-3-phosphate dehydrogenase GAPC4 (gpc4) mRNA, | |
| | | | | | complete cds. | |
| 700346018H1 | g1155259 | 28 | −14 | gb105eukp | Dvir\gl | |
| 700346622H1 | g473976 | 60 | −49 | gb105pln | Rice mRNA, partial homologous to elongation factor 1-alpha gene. | |
| 700345740H1 | g2529657 | 22 | −6 | gb105pln | Arabidopsis thaliana chromosome II BAC T30B22 genomic sequence, complete | |
| | | | | | sequence. | |
| 700281977H2 | g459199 | 83 | −32 | gb105pln | Gossypium hirsutum vacuolar H+-ATPase subunit B mRNA, partial cds. | |
| 700344208H1 | g22319 | 10 | 6 | gb105allp | GSTIII (aa 1-220) | |
| 700346120H1 | g507281 | 11 | −4 | gb105eukp | COXII; cytochrome oxidase subunit 2 | |
| 700344163H1 | g2645166 | 31 | −13 | gb105pln | Oryza sativa mRNA, similar to ubiquitin conjugating enzyme. | |
| 700343968H1 | g2245389 | 13 | 11 | gb105pln | Arabidopsis thaliana auxin response transcription factor 3 (ARF3) mRNA, complete cds. | |
| 700343982H1 | g1616652 | 33 | 0 | gb105allp | calcium-activated potassium channel beta subunit | |
| 700345248H1 | g1321818 | 13 | 4 | gb105allp | RING zinc finger protein | |
| 700345704H1 | g496267 | 34 | −36 | gb105pln | Nicotiana tabacum GTP-binding protein (Ran-A1) mRNA, complete cds. | |
| 700346210H1 | g396840 | 10 | 7 | gb105allp | proline iminopeptidase Pip | |
| 700379905H1 | g1370155 | 22 | −29 | gb105pln | L. japonicus mRNA for small GTP-binding protein, RAB11H. | |
| 700343014H1 | g1100782 | 16 | −5 | gb105eukp | unknown | |
| 700342992H1 | g2599103 | 20 | 1 | gb105pln | Dunaliella salina 60S ribosomal protein (DSRP1) mRNA, complete cds. | |
| 700346256H1 | g484043 | 20 | −3 | gb105allp | ribosomal protein S12 | |
| 700281848H2 | g1107484 | 23 | 4 | gb105pln | A. thaliana mRNA for 40S ribosomal protein S15. | |
| 700342210H1 | g984704 | 45 | −18 | gb105eukp | SPAC24H6.07; unknown | |
| 700345170H1 | g500850 | 33 | −16 | gb105pln | Zea mays (clone pAKHSDH1) aspartate kinase-homoserine dehydrogenase | |
| | | | | | mRNA, complete cds. | |
| 700346439H1 | g1749748 | 30 | −15 | gb105eukp | similar to Saccharomyces cerevisiae eukaryotic initiation factor 4A (EIF-4), | |
| | | | | | SWISS-PROT Accession Number P10081 | |
| 700344321H1 | g1899026 | 26 | −40 | gb105pln | Zea mays superoxide dismutase 4A (sod4A) gene, complete cds. | |
| 700347314H1 | g459894 | 26 | −19 | gb105pln | Zea mays sus1 gene, complete cds. | |
| 700344906H1 | g460042 | 20 | −6 | gb105eukp | DBF3; involved in the control of S phase and pre-mRNA splicing | |
| 700342751H1 | g2245020 | 44 | 6 | gb105allp | growth regulator homolog | |
| 700342724H1 | g2245037 | 12 | 4 | gb105eukp | nuclear antigen homolog | |
| 700342213H1 | g473604 | 17 | −11 | gb105pln | Zea mays W-22 histone H2B mRNA, complete cds. | |
| 700347323H1 | g2245098 | 11 | 0 | gb105eukp | ribosomal protein | |
| 700345561H1 | g1336081 | 23 | −13 | gb105pln | Glycine max var. Century ascorbate peroxidase 2 (APx2) mRNA, | |
| | | | | | complete cds. | |
| 700380182H1 | g2564051 | 27 | −38 | gb105pln | Arabidopsis thaliana genomic DNA, chromosome 5, P1 clone: MWD9, | |
| | | | | | complete sequence. | |
| 700380409H1 | g398326 | 14 | 12 | gb105pln | A. phyllitidis PABP mRNA for poly(A)-binding protein. | |
| 700346838H1 | g2274860 | 22 | −1 | gb105pln | Arabidopsis thaliana mRNA for cytosolic glutathione peroxidase. | |
| 700347086H1 | g2330794 | 9 | 5 | gb105eukp | SPAC24C9.11; hypothetical protein | |
| 700345986H1 | g540534 | 40 | −23 | gb105pln | Rice mRNA for q group of receptor for activated C-kinase, complete cds. | |
| 700347230H1 | g2160300 | 10 | 7 | gb105eukp | ribosomal protein | |
| 700343485H1 | g2827001 | 49 | −25 | gb105pln | Triticum aestivum 70 kDa heat shock protein (TaHSP70d) mRNA, complete cds. | |
| 700346088H1 | g2706466 | 17 | −8 | gb105eukp | SPAC3G9.15c; hypothetical protein | |
| 700344059H1 | g497788 | 10 | 4 | gb105eukp | erd11; glutathione S-transferase; EC 2.5.1.18 | |
| 700342746H1 | g2274990 | 60 | −53 | gb105pln | Hordeum vulgare mRNA for expressed sequence tag. | |
| 700344751H1 | g498774 | 21 | −62 | gb105pln | Z. mays (cv DH5xDH7) hsp70-5 mRNA for heat shock protein 70. | |
| 700343728H1 | g1301996 | 11 | 5 | gb105eukp | ORF YNL092w | |
| 700343159H1 | g1360637 | 38 | −8 | gb105eukp | ENBP1 | |
| 700344979H1 | g710551 | 11 | 4 | gb105allp | ankyrin 3 | |
| 700347024H1 | g168421 | 86 | −72 | gb105pln | Maize auxin-binding protein (ABP) mRNA, complete cds. | |
| 700342882H1 | g168419 | 55 | −2 | gb105pln | Maize (Z. mays) aldolase mRNA, complete cds. | |
| 700346927H1 | g1871360 | 20 | 2 | gb105eukp | glutamyl-prolyl-tRNA synthetase | |
| 700345630H1 | g2564051 | 31 | −9 | gb105pln | Arabidopsis thaliana genomic DNA, chromosome 5, P1 clone: MWD9, complete | |
| | | | | | sequence. | |
| 700343295H1 | g166850 | 15 | −2 | gb105eukp | receptor-like protein kinase; EC 2.7.11 | |
| 700343681H1 | g531055 | 29 | −30 | gb105pln | Wheat mRNA for protein H2B-6, complete cds. | |
| 700343659H1 | g1747293 | 40 | −28 | gb105pln | Rice mRNA for vacuolar H+-pyrophosphatase, complete cds. | |
| 700342456H1 | g2668745 | 35 | −35 | gb105pln | Zea mays inorganic pyrophosphatase (IPP) mRNA, complete cds. | |
| 700342485H1 | g310932 | 36 | −25 | gb105pln | Nicotiana tabacum ribosomal protein L17 mRNA, complete cds. | |
| 700345072H1 | g2827084 | 14 | 4 | gb105eukp | nemdh; malate dehydrogenase precursor; EC 1.1.1.37 | |
| 700344526H1 | g2224913 | 14 | 1 | gb105eukp | beta-expansin | |
| 700379941H1 | g303852 | 64 | −56 | gb105pln | Rice mRNA for ribosomal protein L3, complete cds. | |
| 700379739H1 | g1002380 | 8 | 7 | gb105eukp | RRM-type RNA binding protein | |
| 700343851H1 | g395071 | 24 | −11 | gb105pln | V. faba guanine nucleotide regulatory protein mRNA, complete CDS. | |
| 700347353H1 | g602605 | 97 | −89 | gb105pln | Zea mays tandem genes for alpha1-tubulin and alpha2-tubulin. | |
| 700347061H1 | g2501849 | 41 | −27 | gb105pln | Nicotiana tabacum GDP dissociation inhibitor (GDI) mRNA, complete cds. | |
| 700345801H1 | g1469218 | 32 | −15 | gb105pln | B. oleracea mRNA (unknown). | |
| 700342606H1 | g218488 | 21 | 1 | gb105eukp | Smp2 protein | |
| 700344094H1 | g1431253 | 14 | 6 | gb105pln | Yeast (Saccharomyces cerevisiae) chromosome IV reading frame ORF YDL160c. | |
| 700344179H1 | g22597 | 18 | −0 | gb105eukp | CEX; proline-rich protein | |
| 700344003H1 | g1322620 | 19 | −3 | gb105pln | Yeast (Saccharomyces cerevisiae) chromosome VII reading frame ORF YGL091c. | |
| 700342634H1 | g2642634 | 47 | −3 | gb105eukp | actin | |
| 700342344H1 | g1770190 | 25 | −4 | gb105eukp | cycD1; cyclin-D like protein | |
| 700380728H1 | g1773295 | 12 | 7 | gb105allp | regulatory protein NPR1 | |
| 700347224H1 | g2302292 | 13 | 6 | gb105allp | unnamed protein product | |
| 700343529H1 | g2160155 | 13 | 17 | gb105pln | Sequence of BAC F21M12 from Arabidopsis thaliana chromosome 1, complete | |
| | | | | | sequence. | |
| 700343209H1 | g1652751 | 11 | −4 | gb105allp | DNA mismatch repair protein MutS | |
| 700344073H1 | g2569962 | 11 | 11 | gb105pln | Daucus carota DNA fragment containing transposon-like CACTA/TAGTG repeats | |
| | | | | | and nested inverted repeats. | |
| 700343813H1 | g1223579 | 5 | 8 | gb105eukp | ORF | |
| 700281853H2 | g168490 | 56 | −57 | gb105pln | Maize glutathione S-transferase (GST-I) mRNA, complete cds. | |
| 700342552H1 | g2702406 | 14 | −2 | gb105eukp | C54E4.5 | |
| 700343610H1 | g303854 | 26 | −37 | gb105pln | Rice mRNA for ribosomal protein L7A, complete cds. | |
| 700343559H1 | g21438 | 35 | −28 | gb105pln | S. tuberosum cycl gene encoding cytochrome c1. | |
| 700343035H1 | g306712 | 12 | 8 | gb105allp | putative | |
| 700343215H1 | g473976 | 59 | −44 | gb105pln | Rice mRNA, partial homologous to elongation factor 1-alpha gene. | |
| 700281877H2 | g313147 | 45 | −25 | gb105pln | A. medicago MSK-3 mRNA for protein kinase. | |
| 700344321H1 | g22484 | 42 | −37 | gb105pln | Z. mays RNA for superoxide dismutase Sod4A. | |
| 700380434H1 | g806279 | 8 | −1 | gb105eukp | ribosomal protein | |
| 700342403H1 | g1573909 | 10 | 3 | gb105allp | hypothetical | |
| 700281995H2 | g1707006 | 18 | −3 | gb105pln | Arabidopsis thaliana chromosome II BAC T01B08 genomic sequence, complete | |
| | | | | | sequence. | |
| 700344773H1 | g2827551 | 8 | 4 | gb105eukp | T12H17.130; predicted protein | |
| 700345581H1 | g857573 | 27 | −17 | gb105pln | Oryza sativa vacuolar H+-ATPase (vatp-P1) mRNA, complete cds. | |
| 700344542H1 | g1550660 | 9 | 5 | gb105allp | unknown | |
| 700344001H1 | g577818 | 40 | 12 | gb105pln | Z. mays gene for H2B histone (gH2B4). | |
| 700342827H1 | g290213 | 44 | −25 | gb105eukp | Arf84F; GTP-binding protein | |
| 700379666H1 | g21794 | 25 | −42 | gb105pln | Wheat histone H4 gene. | |
| 700342816H1 | g1072406 | 9 | 6 | gb105allp | unknown | |
| 700281925H1 | g166409 | 19 | 2 | gb105pln | Alfalfa nucleic acid binding protein (alfin-1) mRNA, partial cds. | |
| 700344029H1 | g2547236 | 30 | 1 | gb105allp | DNA mismatch repair protein MSH2 | |
| 700346453H1 | g2462744 | 6 | 7 | gb105eukp | F8A5.28 | |
| 700345268H1 | g606970 | 10 | −2 | gb105eukp | cytoplasmic ribosomal protein L18 | |
| 700347103H1 | g2274990 | 40 | −52 | gb105pln | Hordeum vulgare mRNA for expressed sequence tag. | |
| 700343009H1 | g536895 | 30 | −44 | gb105pln | Wheat mRNA for protein H2A, complete cds, clone wcH2A-10. | |
| 700343084H1 | g170099 | 43 | −36 | gb105pln | Spinach betaine-aldehyde dehydrogenase (BADH) mRNA, complete cds. | |
| 700342333H1 | g2827001 | 30 | −9 | gb105pln | Triticum aestivum 70 kDa heat shock protein (TaHSP70d) mRNA, complete cds. | |
| 700380428H1 | g887795 | 58 | −29 | gb105allp | FXR1 | |
| 700281853H2 | g22314 | 26 | −82 | gb105pln | Maize mRNA for GSH gluthathione S-transferase I (GST; EC 2.5.1.18). | |
| 700344422H1 | g1276768 | 21 | −11 | gb105eukp | rp15; 50S ribosomal protein L5 | |
| 700380650H1 | g170746 | 55 | −42 | gb105pln | Wheat histone H4 TH091 gene, complete cds. | |
| 700342901H1 | g398608 | 26 | 1 | gb105eukp | eEf-1 beta A1; elongation factor-1 beta A1 | |
| 700346361H1 | g535743 | 29 | −6 | gb105pln | Oryza sativa unknown ORF mRNA, complete cds. | |
| 700346351H1 | g17863 | 10 | 6 | gb105allp | r-protein nS15a | |
| 700346467H1 | g634003 | 21 | −3 | gb105eukp | acyl-(acyl carrier protein) thioesterase; EC 3.1.2.14 | |
| 700345959H1 | g1143387 | 30 | −13 | gb105pln | A. thaliana mRNA for Class III ADH. | |
| 700343523H1 | g7364 | 20 | −4 | gb105eukp | ribosomal protein S31 | |
| 700281930H2 | g168498 | 23 | −31 | gb105pln | Corn histone H4 (H4C13) gene, complete cds. | |
| 700380712H1 | g577818 | 29 | −24 | gb105pln | Z. mays gene for H2B histone (gH2B4). | |
| 700347336H1 | g2735839 | 50 | −48 | gb105pln | Sorghum bicolor ADP-glucose pyrophosphorylase subunit SH2, transcriptional | |
| | | | | | regulator, NADPH-dependent reductase A1-a and NADPH-dependent reductase A1-b | |
| | | | | | genes, complete cds. | |
| 700343386H1 | g2293006 | 13 | 6 | gb105allp | hypothetical protein | |
| 700347047H1 | g2661839 | 32 | −16 | gb105pln | Physcomitrella patens mRNA for adenosine kinase. | |
| 700344031H1 | g286254 | 58 | 5 | gb105allp | proteasome subunit R-ZETA | |
| 700345157H1 | g2791806 | 14 | 8 | gb105allp | bet3 | |
| 700342518H1 | g386848 | 71 | −34 | gb105allp | keratin | |
| 700344266H1 | g1370305 | 19 | −11 | gb105eukp | RPL37A | |
| 700343531H1 | g1658314 | 15 | −4 | gb105pln | O. sativa osr40g3 gene. | |
| 700343365H1 | g2258073 | 27 | −5 | gb105pln | Hordeum vulgare var. distichum soluble inorganic pyrophosphatase (Ipp) mRNA, | |
| | | | | | complete cds. | |
| 700345202H1 | g2618602 | 13 | 7 | gb105pln | Arabidopsis thaliana genomic DNA, chromosome 5, P1 clone, MSJ1, complete | |
| | | | | | sequence. | |
| 700346446H1 | g2244764 | 13 | −2 | gb105eukp | AMP-binding protein | |
| 700342303H1 | g2266661 | 43 | −39 | gb105pln | Hordeum vulgare mRNA for 14-3-3 protein (Hv1433c). | |
| 700342955H1 | g2345153 | 40 | −49 | gb105pln | Zea mays Ribsomal protein S4 (rps4) mRNA, complete cds. | |
| 700281913H2 | g2281702 | 42 | −40 | gb105pln | Hordeum vulgare beta-amylase mRNA, partial cds. | |
| 700345433H1 | g21800 | 44 | −21 | gb105pln | T. aestivum L mRNA for histone H2B. | |
| 700379659H1 | g2832245 | 14 | 3 | gb105eukp | hypothetical protein | |
| 700343156H1 | g182651 | 8 | 7 | gb105allp | midkine | |
| 700342669H1 | g623589 | 48 | −38 | gb105pln | Nicotiana tabacum SR1 Nt-rab7b mRNA, complete cds. | |
| 700342629H1 | g2570504 | 45 | −29 | gb105pln | Oryza sativa proteasome component mRNA, complete cds. | |
| 700342619H1 | g168500 | 33 | −44 | gb105pln | Maize (Zea mays) histone H4 gene (H4C14), complete cds. | |
| 700344660H1 | g2464945 | 45 | −8 | gb105eukp | beta-galactosidase | |
| 700342607H1 | g312002 | 35 | −5 | gb105allp | SmE protein | |
| 700346954H1 | g293901 | 23 | −2 | gb105pln | Zea mays Zea mI gene, complete cds. | |
| 700379829H1 | g218241 | 22 | −36 | gb105pln | Rice mRNA for ribosomal protein L3 (T82 gene), partial sequence. | |
| 700346010H1 | g602802 | 29 | −13 | gb105eukp | Dsub\Rp49; ribosomal protein 49 | |
| 700346834H1 | g2833627 | 24 | −9 | gb105pln | Arabidopsis thaliana chromosome 1 BAC F17O7 complete sequence. | |
| 700342755H1 | g1553128 | 15 | 16 | gb105pln | Gossypium hirsutum ribosomal protein L44 isoform a (RL44), complete cds. | |
| 700345889H1 | g2662346 | 70 | −54 | gb105pln | Oryza sativa mRNA for EF-1 alpha, complete cds. | |
| 700379877H1 | g1907232 | 11 | −0 | gb105eukp | YCR094w, len:391 | |
| 700346876H1 | g2662468 | 19 | −13 | gb105pln | Arabidopsis thaliana ribosomal protein S6 (rps6) mRNA, complete cds. | |
| 700345202H1 | g2244973 | 12 | 3 | gb105eukp | similarity to extensin class 1 protein | |
| 700342952H1 | g1066082 | 16 | 1 | gb105allp | DNA-binding protein | |
| 700343684H1 | g1086625 | 10 | 0 | gb105eukp | C06A6.4 | |
| 700345801H1 | g2584827 | 24 | −3 | gb105pln | Sequence of BAC F12F1 from Arabidopsis thaliana chromosome 1, complete | |
| | | | | | sequence. | |
| 700342536H1 | g168502 | 50 | −44 | gb105pln | Maize (Zea mays) histone H4 gene (H4C7), complete cds. | |
| 700380124H1 | g171267 | 15 | 5 | gb105eukp | COX12; cytochrome c oxidase subunit VIb | |
| 700347076H1 | g603808 | 16 | −8 | gb105eukp | STT3; STT3 protein | |
| 700344842H1 | g18266 | 19 | 16 | gb105pln | C. stellata mRNA for ribosomal protein L27. | |
| 700346045H1 | g806807 | 24 | −18 | gb105pln | Pisum sativum chaperonin precursor mRNA, chloroplast gene encoding chloroplast | |
| | | | | | protein, complete cds. | |
| 700343516H1 | g602252 | 67 | −77 | gb105pln | Zea mays enolase (eno2) mRNA, complete cds. | |
| 700347326H1 | g602252 | 77 | −62 | gb105pln | Zea mays enolase (eno2) mRNA, complete cds. | |
| 700345278H1 | g534915 | 29 | −15 | gb105pln | S. tuberosum (Desiree) ppa mRNA for soluble inorganic pyrophosphatase. | |
| 700346641H1 | g416514 | 25 | −13 | gb105allp | NEDD-6 | |
| 700345474H1 | g218175 | 51 | −40 | gb105pln | Rice mRNA for heat shock protein 82 (NH67 gene), partial sequence. | |
| 700343712H1 | g1638838 | 18 | 2 | gb105allp | unknown | |
| 700346102H1 | g2826899 | 22 | −24 | gb105pln | Oryza sativa mRNA for DNA polymerase alpha catalytic subunit, complete cds. | |
| 700344634H1 | g1182048 | 24 | −9 | gb105eukp | nda4; Nda4 | |
| 700379646H1 | g347061 | 22 | 1 | gb105pln | Brassica rapa ribosomal protein L37A (RPL37A) mRNA, complete cds. | |
| 700344059H1 | g676880 | 9 | 4 | gb105allp | The expression is induced by aluminium treatment and Pi starvation. | |
| 700343656H1 | g1945410 | 21 | −20 | gb105eukp | Ap47; clathrin associated protein AP47 | |
| 700344951H1 | g998429 | 37 | −59 | gb105pln | GRF1 = general regulatory factor [Zea mays, XL80, Genomic, 5348 nt]. | |
| 700380584H1 | g2394431 | 9 | 6 | gb105eukp | F23F1.5 | |
| 700346566H1 | g546359 | 22 | −2 | gb105pln | ferric leghemoglobin reductase [Glycine max = soybeans, Merr. nodules, mRNA, 1740 nt]. | |
| 700343802H1 | g1561577 | 30 | −18 | gb105eukp | spermine synthase 1; EC 2.5.1.22 | |
| 700344894H1 | g606970 | 23 | −9 | gb105eukp | cytoplasmic ribosomal protein L18 | |
| 700380088H1 | g474321 | 35 | −20 | gb105pln | Yeast ste13+ gene for RNA helicase, complete cds. | |
| 700346641H1 | g1217609 | 17 | −3 | gb105allp | Csa-19 homlogue | |
| 700345907H1 | g2649663 | 10 | 4 | gb105allp | branched-chain amino acid aminotransferase (ilvE) | |
| 700343707H1 | g1053056 | 17 | −19 | gb105pln | Triticum aestivum histone H3 gene, partial cds, clone W1. | |
| 700344301H1 | g1707657 | 17 | 8 | gb105allp | DnaJ homologue | |
| 700343525H1 | g22483 | 35 | −34 | gb105pln | Z. mays RNA for superoxide dismutase Sod4. | |
| 700344544H1 | g21802 | 43 | −36 | gb105pln | T. aestivum mRNA for high mobility group protein (HMGW). | |
| 700346162H1 | g472913 | 20 | −15 | gb105eukp | Doa; Doa kinase | |
| 700342356H1 | g1914554 | 15 | 4 | gb105eukp | T27E9.2 | |
| 700343590H1 | g218233 | 28 | −11 | gb105pln | Rice mRNA for NDP kinase (T164 gene), partial sequence. | |
| 700346838H1 | g2632108 | 12 | 13 | gb105pln | Pisum sativum mRNA for phospholipid glutathione peroxidase. | |
| 700343483H1 | g2829869 | 51 | −7 | gb105eukp | F3I6.11; pyruvate dehydrogenase E1 alpha subunit | |
| 700343282H1 | g457708 | 29 | −15 | gb105pln | S. oleracea mRNA for protein kinase. | |
| 700379666H1 | g170746 | 25 | −41 | gb105pln | Wheat histone H4 TH091 gene, complete cds. | |
| 700345233H1 | g463856 | 18 | −12 | gb105pln | Chlamydomonas reinhardtii 21 gr ribosomal protein S14 (CRY1) gene, complete cds. | |
| 700342274H1 | g1627599 | 23 | −4 | gb105eukp | C25A1.6 | |
| 700345957H1 | g2564051 | 26 | −8 | gb105pln | Arabidopsis thaliana genomic DNA, chromosome 5, P1 clone, MWD9, complete | |
| | | | | | sequence. | |
| 700344601H1 | g639834 | 28 | −8 | gb105eukp | NADH dehydrogenase; EC 1.6.99.3 | |
| 700344661H1 | g2668737 | 78 | −34 | gb105pln | Zea mays translation initiation factor 5A (TIF5A), mRNA, complete cds. | |
| 700281996H2 | g558648 | 21 | 5 | gb105eukp | D-myo-inositol-3-phosphate synthase; EC 5.5.1.4 | |
| 700343654H1 | g1044992 | 12 | 3 | gb105allp | ribosomal protein L17 | |
| 700342216H1 | g456671 | 33 | −15 | gb105pln | T. aestivum VDAC 1 mRNA. | |
| 700344219H1 | g2827138 | 17 | 2 | gb105pln | Arabidopsis thaliana cellulose synthase catalytic subunit (RSW1) gene, complete cds. | |
| 700342706H1 | g166857 | 48 | −59 | gb105pln | Arabidopsis thaliana cytoplasmic ribosomal protein mRNA, complete cds. | |
| 700342503H1 | g2827001 | 44 | −28 | gb105pln | Triticum aestivum 70 kDa heat shock protein (TaHSP70d) mRNA, complete cds. | |
| 700343188H1 | g218169 | 14 | −1 | gb105pln | Rice mRNA for acyl carrier protein (KN33 gene), partial sequence. | |
| 700342490H1 | g168502 | 74 | −77 | gb105pln | Maize (Zea mays) histone H4 gene (H4C7), complete cds. | |
| 700344038H1 | g36132 | 20 | 5 | gb105allp | rpL32 (aa 1-135) | |
| 700345142H1 | g712823 | 17 | −4 | gb105eukp | PDI; protein disulfide isomerase | |
| 700380548H1 | g1262170 | 19 | −1 | gb105pln | Arabidopsis thaliana phosphoprotein phosphatase 2A regulatory subunit A (RCN1) | |
| | | | | | mRNA, complete cds. | |
| 700347014H1 | g2282583 | 74 | −26 | gb105pln | Zea mays elongation factor 1-alpha (EF1-A) mRNA, complete cds. | |
| 700345687H1 | g531020 | 14 | −0 | gb105eukp | TCP20; chaperonin-like protein | |
| 700343857H1 | g170899 | 14 | 2 | gb105eukp | peroxisomal membrane protein (PMP20A) | |
| 700379825H1 | g1325967 | 25 | −15 | gb105pln | T. aestivum histone H2A gene (clone TH274). | |
| 700379631H1 | g2102696 | 19 | −7 | gb105allp | karyopherin beta 3 | |
| 700343989H1 | g218088 | 49 | −39 | gb105pln | Rice mRNA for ribosomal protein 117 (249 gene), partial sequence. | |
| 700344044H1 | g401845 | 40 | −5 | gb105allp | ribosomal protein L18a | |
| 700344736H1 | g1163029 | 25 | −10 | gb105eukp | T05E11.1 | |
| 700343289H1 | g971283 | 55 | −41 | gb105pln | Rice mRNA for ribosomal protein S31, complete cds. | |
| 700345251H1 | g1272405 | 24 | −5 | gb105pln | Arabidopsis thaliana immunophilin (FKBP15-1) mRNA, complete cds. | |
| 700346880H1 | g1431869 | 13 | 11 | gb105pln | Cucurbita maxima ent-kaurene synthase B mRNA, complete cds. | |
| 700342555H1 | g38223 | 11 | −19 | gb105allp | alpha1-globin | |
| 700343987H1 | g2252863 | 19 | −14 | gb105ekp | A_TM018A10.14 | |
| 700342292H1 | g2662344 | 58 | −45 | gb105pln | Oryza sativa mRNA for EF-1 alpha, complete cds. | |
| 700346267H1 | g533689 | 24 | −2 | gb105pln | Brassica napus Naehan RNA polymerase II subunit RPB10 homolog (BRP10) mRNA, | |
| | | | | | complete cds. | |
| 700342244H1 | g1279761 | 22 | 6 | gb105allp | arfaptin 1 | |
| 700344035H1 | g2654121 | 58 | −28 | gb105pln | Arabidopsis thaliana ribosomal protein L23a (AtrpL23a) mRNA, complete cds. | |
| 700342766H1 | g474002 | 77 | −6 | gb105pln | Rice mRNA, partial homologous to ribosomal protein L39 gene. | |
| 700342872H1 | g1256378 | 11 | 3 | gb105allp | zinc transporter ZnT-2 | |
| 700343807H1 | g945084 | 33 | −24 | gb105pln | Arabidopsis thaliana transcription factor TFIIB mRNA, complete cds. | |
| 700346824H1 | g2369766 | 41 | −13 | gb105eukp | hypothetical protein | |
| 700345625H1 | g668984 | 50 | −37 | gb105pln | S. tuberosum TYKY1 mRNA for NADH:ubiquinone oxidoreductase. | |
| 700345424H1 | g168436 | 48 | −21 | gb105pln | Zea mays catalase (Cat3) gene, complete cds. | |
| 700346539H1 | g2828188 | 28 | −20 | gb105pln | Arabidopsis thaliana genomic DNA, chromosome 5, TAC clone: K3I3, complete | |
| | | | | | sequence. | |
| 700380159H1 | g1899026 | 22 | −36 | gb105pln | Zea mays superoxide dismutase 4A (sod4A) gene, complete cds. | |
| 700346238H1 | g166857 | 47 | −43 | gb105pln | Arabidopsis thaliana cytoplasmic ribosomal protein mRNA, complete cds. | |
| 700343194H1 | g2828187 | 18 | 4 | gb105pln | Arabidopsis thaliana genomic DNA, chromosome 5, TAC clone: K21C13, complete | |
| | | | | | sequence. | |
| 700342791H1 | g732989 | 61 | −20 | gb105pln | Z. mays PCNA mRNA for proliferating cell nuclear antigen. | |
| 700345767H1 | g2330711 | 10 | 6 | gb105eukp | SPAC17H9.04c; hypothetical protein | |
| 700342516H1 | g435174 | 73 | −63 | gb105pln | A. sativa (Pewi) ASTCP-K36 mRNA for t complex polypeptide 1. | |
| 700342360H1 | g2340107 | 73 | −79 | gb105pln | Zea mays starch branching enzyme IIa (Sbe2a) mRNA, partial cds. | |
| 700345277H1 | g170432 | 35 | −32 | gb105pln | Tomato ATP-dependent protease (CD4A) gene, complete cds. | |
| 700345450H1 | g599722 | 20 | 7 | gb105pln | C. melo mRNA for aconitase (UNI-ZAPxR). | |
| 700345831H1 | g185789 | 55 | −31 | gb105allp | immunoglobulin gamma-3 chain | |
| 700345734H1 | g644491 | 45 | −36 | gb105pln | Corn mRNA for elongation factor 1A. | |
| 700343917H1 | g1550813 | 95 | −65 | gb105pln | Z. mays mRNA for acidic ribosomal protein P0. | |
| 700344438H1 | g496661 | 14 | 17 | gb105pln | Yeast (Saccharomyces cerevisiae) genomic DNA, chromosome II from Y element to | |
| | | | | | ILS1 gene. | |
| 700342344H1 | g603509 | 20 | 2 | gb105eukp | cyclin delta-3 | |
| 700344409H1 | g2274990 | 25 | −29 | gb105pln | Hordeum vulgare mRNA for expressed sequence tag. | |
| 700281996H2 | g602565 | 21 | 5 | gb105eukp | INO1 | |
| 700343590H1 | g1777929 | 51 | −39 | gb105pln | Saccharum officinarum nucleoside diphosphate kinase (SoNDPK1) mRNA, | |
| | | | | | complete cds. | |
| 700347308H1 | g2245126 | 11 | 17 | gb105pln | Arabidopsis thaliana DNA chromosome 4, ESSA I contig fragment No. 9. | |
| 700346305H1 | g1750404 | 31 | −8 | gb105eukp | B0412.4 | |
| 700343377H1 | g2645165 | 58 | 0 | gb105pln | Oryza sativa mRNA, similar to ribosomal protein 41. | |
| 700343340H1 | g1469218 | 34 | −17 | gb105pln | B. oleracea mRNA (unknown). | |
| 700344063H1 | g463251 | 33 | −22 | gb105pln | M. sativa (Nagyszenasi) mRNA for ribosomal protein RL5. | |
| 700346970H1 | g452712 | 27 | −12 | gb105eukp | beta-galactosidase; EC 3.2.1.23 | |
| 700343021H1 | g622989 | 16 | −2 | gb105eukp | actin I | |
| 700380179H1 | g473602 | 29 | −15 | gb105pln | Zea mays W-22 histone H2A mRNA, complete cds. | |
| 700379996H1 | g2454309 | 15 | 4 | gb105allp | beta prime COP | |
| 700345538H1 | g643073 | 24 | 8 | gb105pln | Fragaria x ananassa putative 40S ribosomal protein s12 mRNA, complete cds. | |
| 700343924H1 | g167109 | 23 | 3 | gb105pln | Hordeum vulgare vacuolar ATPase B subunit mRNA, complete cds. | |
| 700342796H1 | g2293565 | 54 | −43 | gb105pln | Oryza sativa ADP-ribosylation factor 1 (OLs-ARF1) mRNA, complete cds. | |
| 700346276H1 | g1389566 | 14 | −10 | gb105eukp | ER; receptor protein kinase | |
| 700345632H1 | g550543 | 42 | −27 | gb105pln | A. thaliana mRNA for ribosomal protein L16. | |
| 700281853H2 | g168486 | 62 | −65 | gb105pln | Maize glutathione S-transferase gene (GST-I), exon 1. | |
| 700345113H1 | g2275312 | 32 | 3 | gb105eukp | 40S ribosomal protein S20 homolog | |
| 700345323H1 | g644492 | 82 | −76 | gb105pln | Corn elongation factor 1alpha gene, complete cds. | |
| 700346126H1 | g1771162 | 44 | −16 | gb105eukp | SBT2; serine protease | |
| 700343840H1 | g602605 | 75 | −80 | gb105pln | Zea mays tandem genes for alpha1-tubulin and alpha2-tubulin. | |
| 700344003H1 | g505270 | 19 | −3 | gb105pln | Yeast (Saccharomyces cerevisiae) gene for Nup145p. | |
| 700379791H1 | g902526 | 42 | −34 | gb105pln | Zea mays clone MubG7 ubiquitin fusion protein gene, complete cds. | |
| 700342504H1 | g2832667 | 18 | 10 | gb105pln | Arabidopsis thaliana DNA chromosome 4, BAC clone T10I14 (ESSAII project). | |
| 700344974H1 | g22324 | 33 | −31 | gb105pln | Z. mays mRNA for H2B histone (clone cH2B221). | |
| 700346721H1 | g762850 | 9 | 5 | gb105eukp | wos2; p21 protein | |
| 700346082H1 | g404165 | 43 | −32 | gb105pln | A. thaliana gene for BBC1 protein. | |
| 700343146H1 | g1816587 | 77 | −53 | gb105pln | Zea mays LON2 protease (LON2) mRNA, complete cds. | |
| 700347309H1 | g1835728 | 23 | 1 | gb105pln | Oryza sativa ribosomal protein mRNA, complete cds. | |
| 700343663H1 | g1217651 | 56 | −25 | gb105allp | carbonyl reductase (NADPH) | |
| 700344915H1 | g2262143 | 27 | −11 | gb105eukp | T10P11.10; putative serine/threonine protein kinase/T10P11.10 | |
| 700343018H1 | g1644320 | 14 | 4 | gb105eukp | SPAC6G9.08; putative ubiquitin carboxyl terminal hydrolase | |
| 700343795H1 | g2465746 | 11 | −1 | gb105eukp | trpG; anthranilate synthase component II | |
| 700343917H1 | g18140 | 48 | −29 | gb105pln | C. rubrum mRNA for light-induced 34kD protein. | |
| 700343016H1 | g1200255 | 20 | −3 | gb105pln | S. tuberosum stpk1 gene, and putative sucrase gene. | |
| 700342606H1 | g825570 | 21 | 1 | gb105eukp | SMP2; Smp2p | |
| 700347105H1 | g2511590 | 35 | −9 | gb105eukp | prcgb; multicatalytic endopeptidase complex, proteasome component, beta subunit; | |
| | | | | | EC 3.4.99.46 | |
| 700347111H1 | g443802 | 26 | 1 | gb105allp | ribosomal protein L36 | |
| 700346760H1 | g451192 | 20 | −3 | gb105pln | Triticum aestivum (wali7) mRNA, 3′ end, partial cds. | |
| 700342356H1 | g32101 | 82 | −22 | gb105allp | hinge protein precursor (AA −13 to 78) | |
| 700345932H1 | g707001 | 21 | −0 | gb105eukp | sucrolytic enzyme/ferredoxin homolog | |
| 700343574H1 | g1575129 | 41 | −40 | gb105pln | Zea mays lumenal binding protein cBiPe3 mRNA, complete cds. | |
| 700281821H2 | g899607 | 80 | −75 | gb105pln | Zea mays polyubiquitin (MubC5) mRNA, complete cds. | |
| 700379842H1 | g1167953 | 19 | −22 | gb105eukp | putative 32.6 kDa jasmonate-induced protein | |
| 700380655H1 | g1658314 | 28 | −31 | gb105pln | O. sativa osr40g3 gene. | |
| 700347094H1 | g2342735 | 19 | −2 | gb105eukp | T14G11.28 | |
| 700345588H1 | g1279218 | 39 | −32 | gb105pln | B. napus mRNA for acyl-[acyl-carrier protein] desaturase. | |
| 700342220H1 | g473602 | 82 | −77 | gb105pln | Zea mays W-22 histone H2A mRNA, complete cds. | |
| 700345188H1 | g1402896 | 14 | 4 | gb105allp | cyclin delta-2 | |
| 700342669H1 | g1155264 | 66 | −56 | gb105pln | Pennisetum ciliare possible apospory-associated protein mRNA, complete cds. | |
| 700380141H1 | g340017 | 38 | 3 | gb105allp | alpha-tubulin isotype H2-alpha | |
| 700347264H1 | g1326262 | 35 | −20 | gb105eukp | D1005.1 | |
| 700345568H1 | g1568634 | 22 | −5 | gb105pln | Arabidopsis thaliana AtKAP alpha mRNA, complete cds. | |
| 700345565H1 | g170772 | 63 | −55 | gb105pln | Triticum aestivum S-adenosyl-L-homocysteine hydrolase (SH6.2) mRNA, | |
| | | | | | complete cds. | |
| 700346467H1 | g2459531 | 20 | −2 | gb105eukp | Mf-FatB1; acyl-ACP thioesterase | |
| 700345690H1 | g1405747 | 13 | 1 | gb105allp | PR264/SC35 | |
| 700342936H1 | g16086 | 20 | −22 | gb105pln | A. porrum dnaJ mRNA for DNA J protein (partial). | |
| 700347186H1 | g871831 | 17 | −5 | gb105pln | Arabidopsis thaliana ArLIM15 gene for recA-like protein, complete cds (exon1-15). | |
| 700342343H1 | g2370521 | 19 | −3 | gb105eukp | SPAC2E11.07c; hypothetical atpase | |
| 700344923H1 | g1354206 | 21 | 0 | gb105pln | Arabidopsis thaliana FK506 binding protein FKBP62 (ROF1) mRNA, complete cds. | |
| 700346689H1 | g2529229 | 42 | −7 | gb105eukp | gnd; 6-phosphogluconate dehydrogenase; EC 1.1.1.44 | |
| 700344882H1 | g777757 | 65 | −63 | gb105pln | Saccharum hybrid (clone SCUBI561) polyubiquitin mRNA, complete cds. | |
| 700343995H1 | g633606 | 29 | −17 | gb105pln | P. sativum mRNA for chloroplastic outer envelope membrane protein (OEP75). | |
| 700345183H1 | g21794 | 21 | −10 | gb105pln | Wheat histone H4 gene. | |
| 700342656H1 | g218130 | 26 | −14 | gb105pln | Rice mRNA for Ribosomal protein S15. | |
| 700380745H1 | g587564 | 17 | −2 | gb105eukp | MP; mitochondrial processing peptidase | |
| 700347223H1 | g2660670 | 14 | −1 | gb105eukp | T19K24.9; putative Cu2+-transporting ATPase | |
| 700343567H1 | g435542 | 28 | −20 | gb105pln | Z. mays mRNA for calmodulin. | |
| 700342354H1 | g1061037 | 21 | −3 | gb105pln | A. thaliana mRNA for sterol-C5-desaturase. | |
| 700343282H1 | g457688 | 39 | −26 | gb105pln | M. crystallinum mRNA for protein kinase. | |
| 700281924H2 | g1100224 | 43 | −35 | gb105pln | Pinus sylvestris chloroplast NAD+-dependent glyceraldehyde-3-phosphate | |
| | | | | | dehydrogenase mRNA, complete cds. | |
| 700344647H1 | g1322038 | 7 | 4 | gb105allp | inositol 1,3,4-trisphosphate 5/6-kinase | |
| 700346392H1 | g167968 | 12 | 5 | gb105eukp | CARSR8; glutathione transferase | |
| 700344763H1 | g1185390 | 21 | −1 | gb105eukp | alphacpn60 | |
| 700343858H1 | g595816 | 10 | 4 | gb105eukp | mudrA | |
| 700343952H1 | g22237 | 100 | −90 | gb105pln | Maize mRNA for cytosolic GAPDH (GapC) glyceraldehyde-3-phosphate | |
| | | | | | dehydrogenase. | |
| 700342822H1 | g2104461 | 39 | −21 | gb105eukp | cct7; Cct7p | |
| 700344906H1 | g2440191 | 25 | −15 | gb105eukp | hypothetical protein | |
| 700380141H1 | g9762 | 44 | 4 | gb105allp | alpha-tubulin | |
| 700380714H1 | g13525967 | 24 | −5 | gb105pln | T. aestivum histone H2A gene (clone TH274). | |
| 700346347H1 | g2370557 | 16 | −16 | gb105eukp | SPAC9G1.09; serine threonine protein kinase | |
| 700345740H1 | g303854 | 68 | −65 | gb105pln | Rice mRNA for ribosomal protein L7A, complete cds. | |
| 700344274H1 | g1185156 | 9 | 5 | gb105eukp | rps17; 30S ribosomal protein S17 | |
| 700344447H1 | g407410 | 11 | −11 | gb105eukp | Unknown; 14 kDa polypeptide | |
| 700345878H1 | g293888 | 92 | −76 | gb105pln | Zea mays, glyceraldehyde-3-phosphate dehydrogenase mRNA, 3′ end (clone GAPC2). | |
| 700345709H1 | g902583 | 79 | −84 | gb105pln | Zea mays clone MubG1 ubiquitin gene, complete cds. | |
| 700343820H1 | g395381 | 7 | 5 | gb105allp | elongation factor-1 alpha | |
| 700380531H1 | g218160 | 36 | −26 | gb105pln | Oryza sativa mRNA for elongation actor 1 beta′. | |
| 700345876H1 | g1256771 | 22 | −6 | gb105eukp | COP9 | |
| 700344402H1 | g1762939 | 21 | −9 | gb105eukp | ORF; able to induce HR-like lesions | |
| 700380290H1 | g644492 | 59 | −48 | gb105pln | Corn elongation factor 1alpha gene, complete cds. | |
| 700379957H1 | g736308 | 15 | −1 | gb105eukp | unknown | |
| 700345914H1 | g717080 | 31 | −10 | gb105pln | Zea mays glyceraldehyde-3-phosphate dehydrogenase (GapC4) gene, promoter region. | |
| 700343023H1 | g2811223 | 16 | 7 | gb105pln | Arabidopsis thaliana fimbrin 1 mRNA, partial cds. | |
| 700342888H1 | g289194 | 34 | −17 | gb105pln | Arabidopsis thaliana dihydrofolate reductase-thymidylate synthase (THY-2) mRNA, | |
| | | | | | exons 1 through 9 and complete cds. | |
| 700344631H1 | g728520 | 15 | 6 | gb105pln | A. thaliana (Columbia) cyc3b mRNA for cylcin 3b protein. | |
| 700379682H1 | g1132482 | 35 | −28 | gb105pln | Rice mRNA for ADP-ribosylation factor, complete cds. | |
| 700344895H1 | g1196442 | 51 | −24 | gb105allp | galactose-specific lectin | |
| 700342456H1 | g534915 | 32 | −34 | gb105pln | S. tuberosum (Desiree) ppa mRNA for soluble inorganic pyrophosphatase. | |
| 700342446H1 | g849135 | 54 | −43 | gb105pln | Vigna radiata vacuolar H+-ATPase subunit A mRNA, complete cds. | |
| 700347302H1 | g1183936 | 13 | 14 | gb105pln | P. sativum 5S rRNA gene. | |
| 700344195H1 | g2331281 | 18 | 5 | gb105allp | hamartin | |
| 700346073H1 | g1711035 | 34 | −17 | gb105pln | Pisum sativum hydroxyproline rich glycoprotein PsHRGP1 mRNA, partial cds. | |
| 700343573H1 | g1107564 | 32 | −16 | gb105eukp | RpS13; ribosomal protein S13 | |
| 700343340H1 | g2584827 | 20 | −8 | gb105pln | Sequence of BAC F12F1 from Arabidopsis thaliana chromosome 1, complete | |
| | | | | | sequence. | |
| 700342610H1 | g669002 | 21 | 2 | gb105pln | Glycine max calnexin mRNA, complete cds. | |
| 700347301H1 | g571469 | 8 | 17 | gb105pln | Chlamydomonas reinhardtii histone H3 (ch3-II), histone H4 (ch4-II), histone H2B | |
| | | | | | (ch2b-II) and histone H2A (ch2a-II) genes, complete cds. | |
| 700342453H1 | g170663 | 37 | −27 | gb105pln | Wheat protein kinase mRNA, partial cds. | |
| 700342345H1 | g433041 | 15 | −20 | gb105pln | Zea mays W-22 clone PREM-1B retroelement PREM-1, partial sequence. | |
| 700346143H1 | g2257543 | 16 | −4 | gb105eukp | pi055; protein arginine N-methyltransferase | |
| 700380531H1 | g218340 | 33 | −21 | gb105pln | Triticum aestivum mRNA for elongation factor 1 beta′. | |
| 700379935H1 | g1870202 | 44 | −70 | gb105pln | Z. mays cyp71c3 gene. | |
| 700379711H1 | g168500 | 22 | −22 | gb105pln | Maize (Zea mays) histone H4 gene (H4C14), complete cds. | |
| 700346736H1 | g22117 | 21 | 5 | gb105allp | casein kinase II alpha subunit | |
| 700344941H1 | g1054795 | 30 | −32 | gb105pln | H. vulgare mRNA for transmembrane protein. | |
| 700345103H1 | g2724047 | 22 | −12 | gb105pln | Fritillaria agrestis glutaredoxin type II (glr2) mRNA, complete cds. | |
| 700346745H1 | g927572 | 14 | 6 | gb105eukp | Calreticulin precursor | |
| 700345908H1 | g2827513 | 15 | 12 | gb105pln | Arabidopsis thaliana DNA chromosome 4, BAC clone F8F16 (ESSAII project). | |
| 700346714H1 | g22748 | 11 | 7 | gb105allp | actin depolymerizing factor | |
| 700342634H1 | g1372968 | 52 | −3 | gb105eukp | MocuMA2; actin | |
| 700380473H1 | g22123 | 58 | −59 | gb105pln | Maize alcohol dehydrogenase 1 gene (Adh1-1S). | |
| 700345771H1 | g16379 | 27 | −23 | gb105pln | A. thaliana mRNA for laminin receptor homologue. | |
| 700380763H1 | g832875 | 35 | −29 | gb105pln | Lycopersicon esculentum ascorbate free radical reductase (AFRR), complete cds. | |
| 700343406H1 | g939780 | 27 | −19 | gb105pln | Zea mays MADS-box protein (ZAG5) mRNA, complete cds. | |
| 700380076H1 | g2529661 | 46 | −1 | gb105eukp | T30B22.4; putative dTDP-glucose 4-6-dehydratase | |
| 700343589H1 | g20250 | 46 | −39 | gb105pln | Oryza sativa H3 histone gene H3R-11. | |
| 700343219H1 | g2773153 | 32 | −45 | gb105pln | Oryza sativa abscisic acid- and stress-inducible protein (Asr1) mRNA, complete cds. | |
| 700344217H1 | g397400 | 21 | 8 | gb105pln | B. rapa mRNA for S phase specific gene. | |
| 700346510H1 | g2244934 | 16 | 0 | gb105eukp | similarity to SYNAPTOBREVIN-RELATED PROTEIN | |
| 700345552H1 | g168275 | 35 | −26 | gb105pln | Sweet potato starch phosphorylase mRNA, complete cds. | |
| 700342393H1 | g52783 | 47 | −35 | gb105allp | 47 kd keratin | |
| 700346456H1 | g312575 | 15 | 16 | gb105pln | L. laureoleum 26S rRNA (partial). | |
| 700346207H1 | g2632928 | 13 | 3 | gb105allp | sorbitol dehydrogenase | |
| 700343253H1 | g218082 | 41 | −31 | gb105pln | Rice mRNA for initiation factor eIF-4D (225 gene), partial sequence. | |
| 700342656H1 | g1107484 | 22 | −4 | gb105pln | A. thaliana mRNA for 40S ribosomal protein S15. | |
| 700343414H1 | g558649 | 26 | −4 | gb105pln | T. aestivum VDAC2 mRNA for voltage dependent anion channel. | |
| 700343245H1 | g520543 | 49 | −55 | gb105pln | Sorghum bicolor clone BADH1 betaine aldehyde dehydrogenase mRNA, partial cds. | |
| 700343173H1 | g2088722 | 9 | 5 | gb105allp | strong similarity to the L30P family of ribosomal proteins | |
| 700343589H1 | g168492 | 45 | −39 | gb105pln | Corn histone H3 (H3C3) gene, complete cds. | |
| 700380222H1 | g485985 | 5 | 6 | gb105eukp | ORF YKL009w | |
| 700344248H1 | g712823 | 9 | −2 | gb105eukp | PDI; protein disulfide isomerase | |
| 700346351H1 | g440824 | 10 | 6 | gb105allp | ribosomal protein S15 | |
| 700343367H1 | g2765350 | 10 | 6 | gb105allp | MBNL protein | |
| 700347077H1 | g1865682 | 25 | 13 | gb105pln | A. thaliana 16 kb chromosome 1 DNA fragment. | |
| 700345425H1 | g22121 | 100 | −31 | gb105pln | Maize alcohol dehydrogenase 1 gene (Adh1-1F). | |
| 700346664H1 | g1906825 | 58 | −46 | gb105pln | A. thaliana hsp81.2 gene. | |
| 700343587H1 | g402552 | 21 | −11 | gb105eukp | ketol-acid reductoisomerase; EC 1.1.1.86 | |
| 700346978H1 | g393706 | 38 | −27 | gb105pln | C. sativus mRNA for 3-ketoacyl-CoA thiolase. | |
| 700342743H1 | g2564047 | 15 | 10 | gb105pln | Arabidopsis thaliana genomic DNA, chromosome 5, P1 clone; MJB21, complete | |
| | | | | | sequence. | |
| 700342440H1 | g1220137 | 24 | −23 | gb105eukp | ribosomal protein RL10 | |
| 700379690H1 | g1895083 | 96 | −81 | gb105pln | Zea mays golgi associated protein se-wap41 mRNA, complete cds. | |
| 700380138H1 | g2529341 | 28 | −26 | gb105pln | Spinacia oleracea chloroplast transketolase mRNA, complete cds. | |
| 700379735H1 | g1749509 | 18 | 3 | gb105pln | Fission Yeast mRNA, partical cds. | |
| 700346210H1 | g1710151 | 14 | 3 | gb105eukp | proline iminopeptidase | |
| 700344757H1 | g306819 | 33 | −16 | gb105allp | glutathione transferase M4 | |
| 700346079H1 | g577611 | 55 | −42 | gb105pln | Zea mays CRT1 gene for calcium-binding protein. | |
| 700342736H1 | g556559 | 40 | −28 | gb105pln | Rice mRNA for homlogue of Tat binding protein, complete cds. | |
| 700345587H1 | g563926 | 45 | −33 | gb105pln | Zea mays xyloglucan endo-transglycosylase homolog mRNA, complete cds. | |
| 700343309H1 | g38223 | 60 | −27 | gb105allp | alpha1-globin | |
| 700345694H1 | g2231312 | 37 | −9 | gb105eukp | AtRAB18; AtRab18 | |
| 700342411H1 | g1036803 | 38 | −24 | gb105eukp | TGF-beta receptor interacting protein 1 homolog | |
| 700345637H1 | g20163 | 45 | −34 | gb105pln | O. sativa Rr15 mRNA for 5S ribosomal RNA. | |
| 700342529H1 | g22322 | 26 | −39 | gb105pln | Z. mays mRNA for H2B histone (clone cH2B214). | |
| 700342434H1 | g2150129 | 36 | −20 | gb105pln | Arabidopsis thaliana cytoplasmic ribosomal protein S15a mRNA, complete cds. | |
| 700379628H1 | g311906 | 39 | −23 | gb105pln | N. tabacum mRNA for ras-related GTP-binding protein. | |
| 700345573H1 | g540534 | 40 | −32 | gb105pln | Rice mRNA for q group of receptor for activated C-kinase, complete cds. | |
| 700342392H1 | g1019903 | 38 | −30 | gb105pln | Arabidopsis thaliana cell division cycle protein (CDC48) mRNA, complete cds. | |
| 700347280H1 | g19879 | 14 | 12 | gb105pln | N. tabacum mRNA for heat shock protein 82. | |
| 700345836H1 | g170775 | 76 | −60 | gb105pln | Wheat translation elongation factor 1 alpha-subunit (TEF1) mRNA, complete cds. | |
| 700346164H1 | g510876 | 16 | −2 | gb105eukp | ME1; NADP dependent malic enzyme; EC 1.1.1.40 | |
| 700343240H1 | g57720 | 39 | −9 | gb105allp | ribosomal protein S20 (AA 1-119) | |
| 700342204H1 | g2463334 | 25 | −10 | gb105pln | Oryza sativa mRNA for ribosomal protein S4. | |
| 700343709H1 | g167107 | 46 | −30 | gb105pln | Hordeum vulgare vacuolar ATPase B subunit isoform mRNA, complete cds. | |
| 700342377H1 | g1747418 | 97 | −91 | gb105pln | Z. mays mRNA for eukaryotic initiation factor-5. | |
| 700345031H1 | g2275295 | 18 | 10 | gb105pln | Yeast (Schizosaccharomyces pombe) 60S ribosomal protein L31 homolog mRNA, | |
| | | | | | partial cds. | |
| 700347252H1 | g170455 | 61 | −53 | gb105pln | Tomato heat shock cognate protein 80 gene, 3′ end. | |
| 700343470H1 | g169792 | 64 | −41 | gb105pln | Rice histone 3 gene, complete cds. | |
| 700344743H1 | g198574 | 8 | 3 | gb105allp | hydrophilic protein | |
| 700379624H1 | g22328 | 68 | −54 | gb105pln | Maize mRNA for a high mobility group protein. | |
| 700346981H1 | g432367 | 30 | −14 | gb105pln | Rice mRNA for elongation factor 1 beta, complete cds. | |
| 700344219H1 | g2827143 | 20 | 1 | gb105allp | cellulose synthase catalytic subunit | |
| 700343442H1 | g19539 | 22 | 4 | gb105eukp | ribosomal protein YL16 | |
| 700345245H1 | g7357 | 18 | −5 | gb105eukp | ribosomal protein L7 (AA 1-246) | |
| 700346539H1 | g1518539 | 40 | −36 | gb105pln | Glycine max UDP-glucose dehydrogenase mRNA, complete cds. | |
| 700346948H1 | g347061 | 44 | −33 | gb105pln | Brassica rapa ribosomal protein L37A (RPL37A) mRNA, complete cds. | |
| 700345734H1 | g2662340 | 43 | −37 | gb105pln | Oryza sativa mRNA for EF-1 alpha, complete cds. | |
| 700343059H1 | g927571 | 70 | −85 | gb105pln | Z. mays mRNA for calreticulin precursor. | |
| 700346422H1 | g473602 | 31 | −17 | gb105pln | Zea mays W-22 histone H2A mRNA, complete cds. | |
| 700343681H1 | g473604 | 30 | −38 | gb105pln | Zea mays W-22 histone H2B mRNA, complete cds. | |
| 700345075H1 | g2226414 | 16 | 1 | gb105eukp | SPBC31F10.03; hypothetical protein | |
| 700345333H1 | g1711035 | 35 | −18 | gb105pln | Pisum sativum hydroxyproline rich glycoprotein PsHRGP1 mRNA, partial cds. | |
| 700346430H1 | g168508 | 14 | 15 | gb105pln | Maize oleosin KD18 (KD18; L2) gene, complete cds. | |
| 700344627H1 | g2827001 | 60 | −48 | gb105pln | Triticum aestivum 70 kDa heat shock protein (TaHSP70d) mRNA, complete cds. | |
| 700345701H1 | g398607 | 17 | 1 | gb105pln | A. thaliana mRNA for elongation factor 1 beta. | |
| 700342993H1 | g479146 | 29 | −3 | gb105eukp | putative ATP synthase subunit | |
| 700380791H1 | g13462 | 21 | 4 | gb105allp | ATPase subunit 6 | |
| 700346386H1 | g407500 | 16 | 0 | gb105eukp | unknown | |
| 700343018H1 | g967276 | 14 | −3 | gb105eukp | tgt; tRNA-guanine transglycosylase | |
| 700342285H1 | g1334510 | 15 | −8 | gb105eukp | COI; cytochrome oxidase subunit 1 | |
| 700380725H1 | g2760317 | 19 | −1 | gb105eukp | F1N21.1 | |
| 700343432H1 | g2765417 | 38 | −28 | gb105pln | Pisum sativum GDA-1 mRNA. | |
| 700281813H2 | g2326372 | 21 | −3 | gb105eukp | ISA1; putative arabinose kinase | |
| 700343909H1 | g18259 | 27 | −7 | gb105pln | C. sativus mRNA for cs DnaJ-1. | |
| 700342374H1 | g939778 | 45 | −26 | gb105pln | Zea mays MADS-box protein (ZAG3) mRNA, complete cds. | |
| 700344979H1 | g710552 | 11 | 4 | gb105allp | ankyrin 3 | |
| 700379837H1 | g806299 | 21 | −9 | gb105eukp | unknown protein | |
| 700343810H1 | g736271 | 75 | −67 | gb105pln | O. sativa hsp70 gene for heat shock protein 70. | |
| 700343889H1 | g20185 | 63 | −60 | gb105pln | O. sativa mRNA for calmodulin. | |
| 700347230H1 | g1236272 | 10 | 7 | gb105eukp | putative ribosomal protein L26, most similar to pir: S51347 (68.6% identity in 121 aa | |
| | | | | | overlap) | |
| 700344249H1 | g1488296 | 30 | −1 | gb105pln | Oryza sativa osRAD23 mRNA, complete cds. | |
| 700380044H1 | g2301163 | 9 | 2 | gb105allp | unnamed protein product | |
| 700346958H1 | g871949 | 18 | −0 | gb105eukp | PRE4; proteosome component PRE4 | |
| 700344645H1 | g1420923 | 15 | 16 | gb105pln | Zea mays repressor-like protein (in1) gene, complete cds. | |
| 700344913H1 | g602605 | 100 | −86 | gb105pln | Zea mays tandem genes for alpha1-tubulin and alpha2-tubulin. | |
| 700380492H1 | g2407788 | 5 | 5 | gb105eukp | tipD; tipD | |
| 700379794H1 | g22322 | 27 | −7 | gb105pln | Z. mays mRNA for H2B histone (clone cH2B214). | |
| 700344170H1 | g454913 | 21 | −2 | gb105pln | A. porrum LDJ2 mRNA. | |
| 700345031H1 | g18208 | 19 | 3 | gb105pln | C. reinhardtii mRNA for ribosomal protein L31. | |
| 700346092H1 | g347527 | 18 | −4 | gb105allp | ribosomal protein S3 | |
| 700379940H1 | g2662342 | 71 | −61 | gb105pln | Oryza sativa mRNA for EF-1 alpha, complete cds. | |
| 700343952H1 | g1184771 | 73 | −61 | gb105pln | Zea mays glyceraldehyde-3-phosphate dehydrogenase GAPC2 (gpc2) mRNA, | |
| | | | | | complete cds. | |
| 700346467H1 | g804948 | 21 | −3 | gb105eukp | acyl-(acyl carrier protein) thioesterase; EC 3.1.2.14 | |
| 700345696H1 | g533690 | 43 | 0 | gb105eukp | BRP10 | |
| 700343517H1 | g313142 | 7 | 6 | gb105allp | profilin 3 | |
| 700342425H1 | g2252848 | 14 | 9 | gb105pln | Arabidopsis thaliana BAC TM018A10. | |
| 700344019H1 | g1737221 | 32 | −23 | gb105pln | Pisum sativum BP-80 vacuolar sorting receptor mRNA, complete cds. | |
| 700344781H1 | g1632830 | 28 | −1 | gb105pln | R. communis mRNA (unknown). | |
| 700346269H1 | g2330673 | 5 | 6 | gb105eukp | rp122; 60S ribosomal protein | |
| 700343717H1 | g349210 | 17 | −1 | gb105pln | Arabidopsis thaliana ubiquitin conjugating enzyme mRNA, complete cds. | |
| 700344107H1 | g1707363 | 24 | −6 | gb105pln | A. thaliana mRNA for AATP2. | |
| 700345250H1 | g22324 | 16 | −5 | gb105pln | Z. mays mRNA for H2B histone (clone cH2B221). | |
| 700346636H1 | g21800 | 27 | −33 | gb105pln | T. aestivum L mRNA for histone H2B. | |
| 700343056H1 | g1345367 | 8 | −6 | gb105eukp | AMYB; beta-amylase; EC 3.2.1.2 | |
| 700379729H1 | g493709 | 45 | −33 | gb105pln | Rice mRNA for beta-tubulin, complete cds. | |
| 700347156H1 | g1750404 | 29 | −7 | gb105eukp | B0412.4 | |
| 700345462H1 | g432606 | 39 | −30 | gb105pln | ric1 = ras-related GTP binding protein possessing GTPase activity [Oryza sativa = | |
| | | | | | rice, Yamahoushi, callus, mRNA, 955 nt]. | |
| 700380779H1 | g416256 | 16 | −29 | gb105pln | Rice mRNA for H+-ATPase, partial sequence. | |
| 7u00343257H1 | g495262 | 32 | −22 | gb105pln | P. salina sec61 mRNA. | |
| 700343061H1 | g6815 | 10 | 4 | gb105eukp | R08D7.3 | |
| 700342816H1 | g1314112 | 9 | 6 | gb105eukp | YPR037W; unknown | |
| 700380408H1 | g436782 | 27 | −29 | gb105pln | Rice mRNA for cyc07, complete cds. | |
| 700344526H1 | g293902 | 10 | 3 | gb105eukp | Zea mI | |
| 700342622H1 | g1698669 | 98 | −38 | gb105pln | Zea mays S-like RNase (kin1) mRNA, complete cds. | |
| 700344610H1 | g1524370 | 11 | −1 | gb105eukp | tom20; protein import receptor; TOM20 | |
| 700342307H1 | g2370521 | 31 | −13 | gb105eukp | SPAC2E11.07c; hypothetical atpase | |
| 700342634H1 | g1064821 | 52 | −4 | gb105eukp | MocuMA1; larval muscle specific; actin | |
| 700380180H1 | g1931640 | 15 | −5 | gb105eukp | T19D16.4; Serine carboxypeptidase isolog | |
| 700343989H1 | g310932 | 44 | −32 | gb105pln | Nicotiana tabacum ribosomal protein L17 mRNA, complete cds. | |
| 700342669H1 | g2317873 | 50 | −40 | gb105pln | Prunus armeniaca Rab7 GTP binding protein mRNA, complete cds. | |
| 700343622H1 | g1658312 | 20 | −6 | gb105pln | O. sativa osr40g2 gene. | |
| 700380163H1 | g972169 | 19 | −2 | gb105allp | 60S acikdic ribosomal protein P1 | |
| 700346909H1 | g313151 | 28 | −13 | gb105pln | A. thaliana ribosomal protein S15 mRNA, complete CDS. | |
| 700346096H1 | g515376 | 48 | 6 | gb105pln | L. temulentum mRNA for histone H4. | |
| 700346940H1 | g1199467 | 35 | −16 | gb105eukp | CER1; possible aldehyde decarbonylase | |
| 700345770H1 | g2809251 | 51 | −29 | gb105eukp | F21B7.20 | |
| 700343570H1 | g2276355 | 23 | 3 | gb105eukp | SPBC30D10.06; hypothetical protein | |
| 700342962H1 | g218227 | 25 | −0 | gb105pln | Rice mRNA for ras-related GTP binding protein, complete cds. | |
| 700345320H1 | g881635 | 27 | −21 | gb105eukp | Arp3 | |
| 700342582H1 | g2392021 | 19 | −1 | gb105eukp | phopholipid hydroperoxide glutathione peroxidase-like protein, EC 1.11.1.9 | |
| 700342695H1 | g2465430 | 22 | −3 | gb105eukp | JRG1.3; 32 kDa protein | |
| 700343373H1 | g1808684 | 20 | 4 | gb105allp | hypothetical protein | |
| 700347165H1 | g2624379 | 23 | −8 | gb105eukp | IPP1; inorganic pyrophosphatase (pyrophosphate phospho-hydrolase); EC 3.6.1.1 | |
| 700346554H1 | g600004 | 11 | 1 | gb1053ukp | unknown | |
| 700344341H1 | g2618603 | 11 | 16 | gb105pln | Arabidopsis thaliana genomic DNA, chromosome 5, P1 clone; MSL3, complete | |
| | | | | | sequence. | |
| 700346958H1 | g4223 | 18 | −0 | gb105eukp | PRE4; proteasome Pre4 subunit | |
| 700343030H1 | g498772 | 43 | −51 | gb105pln | Z. mays (cv DH5xDH7) hsp70-4 mRNA for heat shock protein 70. | |
| 700342895H1 | g218088 | 62 | −13 | gb105pln | Rice mRNA for ribosomal protein 117 (249 gene), partial sequence. | |
| 700342273H1 | g704447 | 7 | 7 | gb105allp | NADH dehydrogenase subunit 5 | |
| 700344448H1 | g398848 | 32 | −46 | gb105pln | Z. mays mRNA for beta 5 tubulin. | |
| 700345784H1 | g1408460 | 31 | −6 | gb105eukp | type 2A protein serine/threonine phosphatase 55 kDa B regulatory subunit | |
| 700345556H1 | g971699 | 6 | 5 | gb105allp | ribosomal protein S7 | |
| 700343313H1 | g1532214 | 17 | 7 | gb105eukp | cypA; cyclophilin A | |
| 700343930H1 | g1183936 | 15 | 3 | gb105pln | P. sativum 5S rRNA gene. | |
| 700345237H1 | g643479 | 15 | 3 | gb105eukp | GCN20; Gcn20p | |
| 700346090H1 | g2558943 | 35 | −37 | gb105pln | Gossypium hirsutum histone 3 mRNA, complete cds. | |
| 700379874H1 | g576998 | 12 | 1 | gb105allp | C08B11.5 | |
| 700343584H1 | g577611 | 47 | −48 | gb105pln | Zea mays CRT1 gene for calcium-binding protein. | |
| 700342882H1 | g20203 | 44 | 4 | gb105pln | Rice mRNA for fructose-diphosphate aldolase (EC 4.1.2.13). | |
| 700346386H1 | g172551 | 16 | 0 | gb105eukp | SDH1; succinate dehydrogenase flavoprotein subunit | |
| 700342236H1 | g415906 | 22 | −10 | gb105eukp | ORF YKR406 | |
| 700342844H1 | g415316 | 28 | −8 | gb105pln | Rice mRNA for acidic ribosomal protein P0, complete cds. | |
| 700343659H1 | g285637 | 60 | −46 | gb105pln | Hordeum vulgare mRNA for vacuolar membrane proton-translocating inorganic | |
| | | | | | pyrophosphat. | |
| 700346558H1 | g780196 | 14 | 2 | gb105eukp | K01C8.10 | |
| 700342643H1 | g2645163 | 52 | −19 | gb105pln | Oryza sativa mRNA, similar to ribosomal protein L26. | |
| 700344951H1 | g168602 | 76 | −69 | gb105pln | Zea mays regulatory protein GF14-12 mRNA, complete cds. | |
| 700380256H1 | g1562574 | 16 | 2 | gb105allp | U1 snRNP-specific protein C | |
| 700379782H1 | g557681 | 46 | −25 | gb105pln | Z. mays mRNA for ribosomal protein S28. | |
| 700347124H1 | g3075 | 10 | 6 | gb105pln | N. crassa mRNA for a ribosomal protein. | |
| 700346020H1 | g5151 | 26 | −4 | gb105pln | Yeast (Schizosaccharomyces pombe) ypt3 gene. | |
| 700342389H1 | g168500 | 62 | −46 | gb105pln | Maize (Zea mays) histone H4 gene (H4C14), complete cds. | |
| 700342519H1 | g459199 | 57 | −49 | gb105pln | Gossypium hirsutum vacuolar H+-ATPase subunit B mRNA, partial cds. | |
| 700380070H1 | g473987 | 54 | −19 | gb105pln | Rice mRNA, partial homologous to histone H3 gene. | |
| 700346546H1 | g473602 | 66 | −54 | gb105pln | Zea mays W-22 histone H2A mRNA, complete cds. | |
| 700343763H1 | g2145356 | 14 | −5 | gb105eukp | ATHB-14; HD-Zip protein | |
| 700344247H1 | g1498389 | 66 | −59 | gb105pln | Zea mays actin (Maz81) gene, partial cds. | |
| 700344851H1 | g21800 | 15 | −2 | gb105pln | T. aestivum L mRNA for histone H2B. | |
| 700380473H1 | g168405 | 57 | −59 | gb105pln | maize alcohol dehydrogenase (adh1) mrna 3′ end and flank. | |
| 700342754H1 | g2266661 | 42 | −28 | gb105pln | Hordeum vulgare mRNA for 14-3-3 protein (Hv1433c). | |
| 700346465H1 | g2394434 | 53 | −30 | gb105eukp | F23F1.8 | |
| 700343718H1 | g1420584 | 22 | −3 | gb105pln | Yeast (Saccaromyces cerevisiae) chromosome XV reading frame ORF YOR259c. | |
| 700344389H1 | g1420686 | 8 | 1 | gb105allp | ORF YOR312c | |
| 700342876H1 | g1550813 | 85 | −74 | gb105pln | Z. mays mRNA for acidic ribosomal protein P0. | |
| 700345237H1 | g2522534 | 12 | 6 | gb105allp | TNF-alpha stimulated ABC protein | |
| 700380728H1 | g1916912 | 12 | 7 | gb105eukp | nim1; transcription factor inhibitor I kappa B homolog | |
| 700343045H1 | g902597 | 55 | −25 | gb105allp | MIF homologue | |
| 700379952H1 | g1431332 | 20 | −18 | gb105eukp | ORF YDL201w | |
| 700343982H1 | g2209017 | 33 | 0 | gb105allp | potassium channel beta subunit | |
| 700380133H1 | g21800 | 15 | −6 | gb105pln | T. aestivum L mRNA for histone H2B. | |
| 700346840H1 | g166409 | 22 | −2 | gb105pln | Alfalfa nucleic acid binding protein (alfin-1) mRNA, partial cds. | |
| 700344808H1 | g2642217 | 22 | 5 | gb105eukp | NOI protein | |
| 700380408H1 | g217902 | 21 | −20 | gb105pln | C. roseus (periwinkle) cyc07 gene. | |
| 700345737H1 | g1304599 | 6 | 7 | gb105allp | ZNF127-Xp | |
| 700343770H1 | g1495339 | 16 | −5 | gb105eukp | F26A3.3 | |
| 700344366H1 | g2264307 | 16 | −6 | gb105pln | Arabidopsis thaliana genomic DNA, chromosome 5, P1 clone: MED24, complete | |
| | | | | | sequence. | |
| 700343673H1 | g397958 | 16 | 10 | gb105pln | Z. mays mRNA for beta-amylase. | |
| 700345019H1 | g2149050 | 42 | −26 | gb105pln | Arabidopsis thaliana small Ras-like GTP-binding protein (Ran3) mRNA, | |
| | | | | | complete cds. | |
| 700345974H1 | g313759 | 98 | −29 | gb105pln | Z. mays hsp 70-1 gene for heat shock protein 70. | |
| 700342809H1 | g2289960 | 47 | −37 | gb105pln | Arabidopsis thaliana GTPase AtRAB8 (atrab8) mRNA, complete cds. | |
| 700380124H1 | g30295 | 12 | 5 | gb105allp | cytochrome c oxidase subunit BIb (AA 1-86) | |
| 700346592H1 | g2735841 | 11 | 5 | gb105eukp | transcriptional regulator | |
| 700343660H1 | g532650 | 53 | −55 | gb105pln | Sparganium eurycarpum Engl. 18S ribosomal RNA (18S rRNA) gene, 5′ end. | |
| 700343886H1 | g1778442 | 53 | −19 | gb105eukp | PK5; putative protein kinase PK5 | |
| 700345016H1 | g1806140 | 62 | −8 | gb105eukp | cdc2MsC | |
| 700342237H1 | g5303 | 6 | 5 | gb105eukp | PRD1 | |
| 700343008H1 | g914872 | 19 | 0 | gb105pln | Yeast (Saccharomyces cerevisiae) chromosome IV cosmid 6652. | |
| 700342785H1 | g19325 | 38 | 10 | gb105pln | L. esculentum pT62 mRNA for ribosomal RNA internal fragment, homologous to 26S | |
| | | | | | ribosomal RNA. | |
| 700344619H1 | g960289 | 15 | 5 | gb105allp | anthranilate synthase alpha subunit | |
| 700347166H1 | g218130 | 30 | −13 | gb105pln | Rice mRNA for Ribosomal protein S15. | |
| 700346885H1 | g2582506 | 25 | −2 | gb105eukp | beta2_dm; 20S proteasome beta2 subunit | |
| 700380595H1 | g340520 | 13 | −1 | gb105pln | Coptis japonica triosphosphate isomerase mRNA, complete cds. | |
| 700380151H1 | g758246 | 31 | −33 | gb105pln | Phaenopsis sp. mRNA for S-adenosylhomocysteine hydrolase. | |
| 700281809H2 | g2286150 | 45 | −48 | gb105pln | Zea mays translation initiation factor (eIF-4A) mRNA, complete cds. | |
| 700380457H1 | g435678 | 11 | 14 | gb105pln | L. esculentum Mill (cv. Rutgers) mRNA for ribosomal protein S25. | |
| 700344763H1 | g415925 | 22 | −0 | gb105eukp | chaperonin-60 alpha subunit | |
| 700345641H1 | g22324 | 38 | −28 | gb105pln | Z. mays mRNA for H2B histone (clone cH2B221). | |
| 700380194H1 | g13462 | 25 | −6 | gb105allp | ATPase subunit 6 | |
| 700345690H1 | g337926 | 12 | 4 | gb105allp | splicing factor | |
| 700342484H1 | g312015 | 12 | 7 | gb105allp | SOF1 | |
| 700342788H1 | g312083 | 6 | 1 | gb105allp | NUO-10.5 gene product | |
| 700344766H1 | g292037 | 43 | −22 | gb105allp | helix-loop-helix phosphoprotein | |
| 700346248H1 | g1519250 | 43 | −32 | gb105pln | Oryza sativa GF14-c protein mRNA, complete cds. | |
| 700344689H1 | g295737 | 34 | 4 | gb105allp | V14 gene product | |
| 700343995H1 | g576506 | 29 | −17 | gb105pln | Pisum sativum outer membrane protein (IAF75) mRNA, complete cds. | |
| 700342565H1 | g603269 | 14 | −7 | gb105eukp | YER036C; Yer036cp | |
| 700344709H1 | g473976 | 26 | −34 | gb105pln | Rice mRNA, partial homologous to elongation factor 1-alpha gene. | |
| 700380781H1 | g577824 | 23 | −43 | gb105pln | Z. mays gene for H2B histone (gH2B3). | |
| 700342637H1 | g404070 | 12 | 4 | gb105eukp | O-methyltransferase | |
| 700344019H1 | g1737217 | 29 | −19 | gb105pln | Arabidopsis thaliana vacuolar sorting receptor homolog mRNA, complete cds. | |
| 700343373H1 | g2760327 | 16 | 7 | gb105eukp | F1N21.12 | |
| 700346294H1 | g303852 | 24 | −42 | gb105pln | Rice mRNA for ribosomal protein L3, complete cds. | |
| 700345019H1 | g395071 | 47 | −32 | gb105pln | V. faba guanine nucleotide regulatory protein mRNA, complete CDS. | |
| 700343596H1 | g168490 | 79 | −60 | gb105pln | Maize glutathione S-transferase (GST-I) mRNA, complete cds. | |
| 700347216H1 | g2511590 | 29 | −5 | gb105eukp | progb; multicatalytic endopeptidase complex, proteasome component, beta subunit; | |
| | | | | | EC 3.4.99.46 | |
| 700345779H1 | g1197072 | 6 | 7 | gb105eukp | GEF1 | |
| 700343740H1 | g2789434 | 10 | −3 | gb105eukp | CLB1 | |
| 700344335H1 | g22324 | 53 | −48 | gb105pln | Z. mays mRNA for H2B histone (clone cH2B221). | |
| 700345803H1 | g303848 | 41 | −27 | gb105pln | Rice mRNA for nucleoside diphosphate kinase, complete cds. | |
| 700380417H1 | g433216 | 12 | 4 | gb105pln | Rice mRNA for ascorbate peroxidase (gene name SS622), partial cds. | |
| 700345805H1 | g172221 | 18 | 1 | gb105eukp | proteolipid protein of proton ATPase | |
| 700346252H1 | g416265 | 19 | 3 | gb105pln | Rice mRNA for ribosomal protein A2, partial sequence. | |
| 700343525H1 | g22484 | 65 | −66 | gb105pln | Z. mays RNA for superoxide dismutase Sod4A. | |
| 700281862H2 | g2651310 | 9 | 4 | gb105eukp | T2P4.19; putative PTR2-B peptide transporter | |
| 700379945H1 | g949877 | 80 | −69 | gb105pln | H. vulgare mRNA for elongation factor 1-alpha. | |
| 700343063H1 | g2828187 | 15 | 6 | gb105pln | Arabidopsis thaliana genomic DNA, chromosome 5, TAC clone: K21C13, complete | |
| | | | | | sequence. | |
| 700379919H1 | g600768 | 48 | −36 | gb105pln | Oryza sativa cyclophilin 2 (Cyp2) mRNA, complete cds. | |
| 700345480H1 | g2190550 | 30 | −11 | gb105eukp | F5I14.18 | |
| 700342736H1 | g732814 | 30 | −13 | gb105pln | L. esculentum LeMA-1 mRNA for putative Mg-dependent ATPase 1. | |
| 700344095H1 | g1049252 | 85 | −77 | gb105pln | Zea mays vacuolar ATPase 69 kDa subunit mRNA, partial cds. | |
| 700345268H1 | g1550725 | 8 | 7 | gb105eukp | ribosomal protein L18 | |
| 700380791H1 | g1332386 | 24 | 0 | gb105allp | Rattus norvegicus ATPase subunit 6 | |
| 700281960H2 | g22379 | 79 | −89 | gb105pln | Z. mays mRNA for CAAT-box DNA binding protein subunit B (NF-YB). | |
| 700343623H1 | g2511747 | 19 | −5 | gb105eukp | dre4; probable transcriptional regulator dre4 | |
| 700346488H1 | g2257742 | 34 | −20 | gb105pln | Arabidopsis thaliana lysine-sensitive aspartate kinase mRNA, complete cds. | |
| 700342962H1 | g218203 | 37 | −17 | gb105pln | Rice mRNA for GTP binding protein. | |
| 700342695H1 | g2465426 | 22 | −3 | gb105eukp | JRG1.1; 32 kDa protein | |
| 700342367H1 | g248336 | 36 | −48 | gb105pln | polyubiquitin [maize, Genomic, 3841 nt]. | |
| 700380434H1 | g303853 | 9 | −1 | gb105eukp | ribosomal protein L3 | |
| 700343819H1 | g170746 | 60 | −51 | gb105pln | Wheat histone H4 TH091 gene, complete cds. | |
| 700345906H1 | g2258315 | 10 | 7 | gb105allp | resistance complex protein I2C-1 | |
| 700345080H1 | g2258073 | 30 | −32 | gb105pln | Hordeum vulgare var. distichum soluble inorganic pyrophosphatase (Ipp) mRNA, | |
| | | | | | complete cds. | |
| 700345826H1 | g168650 | 35 | −52 | gb105pln | Zea mays ubiquitin fusion protein (UBF9) gene, complete cds. | |
| 700343517H1 | g313138 | 8 | 6 | gb105eukp | profilin 1 | |
| 700344702H1 | g460990 | 59 | −48 | gb105pln | O. sativa (Arborio) Beta Tubulin mRNA, clone OSTB-16. | |
| 700344980H1 | g167109 | 22 | 4 | gb105pln | Hordeum vulgare vacuolar ATPase B subunit mRNA, complete cds. | |
| 700342392H1 | g1669659 | 48 | −37 | gb105pln | c. annuum mRNA for CDC48p-like protein. | |
| 700342401H1 | g2739376 | 9 | 4 | gb105eukp | T9J22.18; putative permease | |
| 700342638H1 | g736271 | 41 | −32 | gb105pln | O. sativa hsp70 gene for heat shock protein 70. | |
| 700346341H1 | g549983 | 14 | −15 | gb105pln | Pennisetum ciliare possible apospory-associated mRNA clone pSUB C, complete cds. | |
| 700346703H1 | g514317 | 27 | −12 | gb105pln | Arabidopsis thaliana RNA polymerase II third largest subunit (RPB35.5A) gene, | |
| | | | | | complete cds. | |
| 700343167H1 | g2842482 | 15 | −5 | gb105eukp | F21O9.80; protein phosphatase ZC-like protein | |
| 700346820H1 | g558649 | 23 | −1 | gb105pln | T. aestivum VDAC2 mRNA for voltage dependent anion channel. | |
| 700343168H1 | g2736379 | 19 | −5 | gb105eukp | T22B11.5 | |
| 700345695H1 | g2662342 | 66 | −61 | gb105pln | Oryza sativa mRNA for EF-1 alpha, complete cds. | |
| 700379937H1 | g248336 | 62 | −52 | gb105pln | polyubiquitin [maize, Genomic, 3841 nt]. | |
| 700346336H1 | g2828188 | 36 | −24 | gb105pln | Arabidopsis thaliana genomic DNA, chromosome 5, TAC clone: K3I3, complete | |
| | | | | | sequence. | |
| 700344982H1 | g1431377 | 7 | 1 | gb150eukp | ORF YDL224c | |
| 700343438H1 | g603311 | 19 | −6 | gb105eukp | RPS24EA; Rps24eap: 40S ribosomal protein S24E (RP50) | |
| 700343989H1 | g2341023 | 45 | −32 | gb105pln | Sequence of BAC F19P19 from Arabidopsis thaliana chromosome 1, complete | |
| | | | | | sequence. | |
| 700344509H1 | g1107460 | 57 | −45 | gb105pln | Rice mRNA for aspartate kinase-homoserine dehydrogenase, complete cds. | |
| 700380180H1 | g2443888 | 14 | −6 | gb105eukp | F11P17.14 | |
| 700346567H1 | g31218 | 19 | 2 | gb105allp | KDEL receptor | |
| 700347263H1 | g606970 | 6 | 8 | gb105allp | cytoplasmic ribosomal protein L18 | |
| 700346954H1 | g2224914 | 37 | −27 | gb105pln | Oryza sativa beta-expansin mRNA, complete cds. | |
| 700345805H1 | g500700 | 18 | 1 | gb105eukp | PPA1; Ppa1p: Proteolipid protein of proton ATPase | |
| 700344894H1 | g4366 | 16 | 1 | gb105eukp | rp 28 | |
| 700380406H1 | g886739 | 55 | −53 | gb105pln | Z. mays histone H4 gene. | |
| 700281844H2 | g710626 | 9 | 6 | gb105eukp | ERD15; ERD15 protein | |
| 700346558H1 | g1431223 | 16 | −8 | gb105eukp | CCT4 | |
| 700342647H1 | g1321660 | 43 | −20 | gb105pln | Rice mRNA for ascorbate peroxidase, complete cds. | |
| 700346653H1 | g2656031 | 25 | −6 | gb105pln | Arabidopsis thaliana genomic DNA, chromosome 5, P1 clone: MXC20. | |
| 700345552H1 | g217998 | 32 | −18 | gb105pln | Potato tuber, alpha-glucan phosphorylase (EC 2.4.1.1), type L isozyme gene, | |
| | | | | | complete cds. | |
| 700346366H1 | g1399563 | 25 | −43 | gb105pln | Hydrastis canedensis nuclear 26S ribosomal RNA gene, partial sequence. | |
| 700342427H1 | g395078 | 38 | −21 | gb105pln | B. rapa ubiquitin and ribosomal protein mRNA, complete CDS's. | |
| 700342644H1 | g532128 | 9 | −3 | gb105eukp | eas; ethanolamine kinase | |
| 700343980H1 | g22324 | 81 | −70 | gb105pln | Z. mays mRNA for H2B histone (clone cH2B221). | |
| 700345336H1 | g17734 | 4 | 6 | gb105eukp | beta-1,3-glucanase homlogue | |
| 700344441H1 | g217951 | 22 | −13 | gb105eukp | S6PDH; NADP-dependent D-sorbitol-6-phosphate dehydrogenase | |
| 700380178H1 | g168543 | 94 | −33 | gb105pln | Zea mays putative ribosomal protein S8 mRNA, partial cds. | |
| 700343905H1 | g2760167 | 24 | −5 | gb105pln | Arabidopsis thaliana genomic DNA, chromosome 5, P1 clone: MCO15, complete | |
| | | | | | sequence. | |
| 700343091H1 | g533689 | 30 | −6 | gb105pln | Brassica napus Naehan RNA polymease II subunit RPB10 homolog (BRP10) mRNA, | |
| | | | | | complete cds. | |
| 700344740H1 | g2274801 | 20 | −7 | gb105eukp | ribosomal protein L4 | |
| 700343477H1 | g2352930 | 14 | 12 | gb105pln | Petroselinum crispum cytosolic chorismate mutase 2 (cCM2) mRNA, partial cds. | |
| 700342830H1 | g19701 | 25 | −14 | gb105pln | N. plumbaginifolia mRNA NeIF-5A2 for initiation factgor 5A(2). | |
| 700342893H1 | g1167953 | 12 | −2 | gb105eukp | putative 32.6 kDa jasmonate-induced protein | |
| 700344895H1 | g179531 | 51 | −24 | gb105allp | IgE-binding protein | |
| 700343401H1 | g217973 | 17 | −31 | gb105pln | Zea mays gene for triosephosphate isomerase, complete cds. | |
| 700343165H1 | g145769 | 13 | 2 | gb105allp | heat shock protein dnaJ | |
| 700344133H1 | g168490 | 76 | −62 | gb105pln | Maize glutathione S-transferase (GST-I) mRNA, complete cds. | |
| 700345993H1 | g2827618 | 20 | −7 | gb105pln | Arabidopsis thaliana DNA chromosome 4, BAC clone F10N7 (ESSAII project). | |
| 700345108H1 | g16086 | 40 | −31 | gb105pln | A. porrum dnaJ mRNA for DNA J protein (partial). | |
| 700379994H1 | g2687432 | 45 | −61 | gb105pln | Plumbago auriculata large subunit 26S ribosomal RNA gene, partial sequence. | |
| 700346734H1 | g515758 | 48 | −42 | gb105pln | Vitis vinifera (clone pGME1) malate dehydrogenase mRNA, complete cds. | |
| 700343516H1 | g22272 | 31 | −16 | gb105pln | Maize mRNA for enolase (2-phospho-D-glycerate hydrolase). | |
| 700342960H1 | g182035 | 60 | −45 | gb105allp | elastase III B | |
| 700379601H1 | g170455 | 42 | −20 | gb105pln | Tomato heat shock cognate protein 80 gene, 3′ end. | |
| 700343729H1 | g2244950 | 9 | 15 | gb105pln | Arabidopsis thaliana DNA chromosome 4, ESSA I contig fragment No. 5. | |
| 700380714H1 | g473602 | 60 | −55 | gb105pln | Zea mays W-22 histone H2A mRNA, complete cds. | |
| 700380214H1 | g167687 | 19 | −4 | gb105pln | Dictyostelium discoideum clathrin heavy chain (chcA) mRNA, complete cds. | |
| 700347312H1 | g1050540 | 32 | −13 | gb105eukp | tRNA-glutamine synthetase | |
| 700347134H1 | g452473 | 51 | −55 | gb105pln | Zea mays Black Mexican Sweet alpha-tubulin mRNA, complete cds. | |
| 700346512H1 | g2191184 | 33 | −12 | gb105eukp | A_TM021B04.4 | |
| 700346969H1 | g2781362 | 6 | 1 | gb105eukp | F24O1.18 | |
| 700346057H1 | g22322 | 27 | −42 | gb105pln | Z. mays mRNA for H2B histone (clone cH2B214). | |
| 700344524H1 | g1864000 | 43 | −59 | gb105pln | Maize DNA for Fd III, complete cds. | |
| 700380064H1 | g436782 | 47 | −32 | gb105pln | Rice mRNA for cyc07, complete cds. | |
| 700347261H1 | g777757 | 40 | −45 | gb105pln | Saccharum hybrid (clone SCUBI561) polyubiquitin mRNA, complete cds. | |
| 700346118H1 | g4026 | 15 | 3 | gb105eukp | NAM8 | |
| 700345559H1 | g454881 | 21 | −22 | gb105pln | Rice gene for thioredoxin h, complete cds. | |
| 700343822H1 | g2832242 | 38 | −32 | gb105pln | Zea mays 22-kDa alpha zein gene cluster, complete sequence. | |
| 700343844H1 | g1184771 | 95 | −79 | gb105pln | Zea mays glyceraldehyde-3-phosphate dehydrogenase GAPC2 (gpc2) mRNA, | |
| | | | | | complete cds. | |
| 700345847H1 | g498754 | 21 | −4 | gb105eukp | YBR0309 | |
| 700345516H1 | g2749918 | 13 | 17 | gb105pln | Arabidopsis thaliana chromosome I BAC F3I6 genomic sequence, complete sequence. | |
| 700342650H1 | g439585 | 68 | −32 | gb105pln | Hordeum vulgare calreticulin (CRH1) mRNA, partial cds. | |
| 700342489H1 | g726422 | 14 | 0 | gb105eukp | F08B1.1 | |
| 700344966H1 | g218170 | 22 | −24 | gb105pln | Rice mRNA for adenylate kinase (KN57 gene), partial sequence. | |
| 700380613H1 | g22324 | 40 | −37 | gb105pln | Z. mays mRNA for H2B histone (clone cH2B221). | |
| 700345742H1 | g2760330 | 17 | −13 | gb105eukp | F1N21.15 | |
| 700379905H1 | g1370157 | 27 | −28 | gb105pln | L. japonicus mRNA for small GTP-binding protein, RAB11I. | |
| 700345784H1 | g710330 | 27 | −4 | gb105eukp | 55 kDa B regulatory subunit of phosphatase 2A | |
| 700345817H1 | g517492 | 17 | 13 | gb105pln | Z. mays Zm38 gene, intron 1. | |
| 700344378H1 | g1184771 | 70 | −79 | gb105pln | Zea mays glyceraldehyde-3-phosphate dehydrogenase GAPC2 (gpc2) mRNA, | |
| | | | | | complete cds. | |
| 700346012H1 | g2257543 | 22 | 0 | gb105eukp | pi055; protein arginine N-methyltransferase | |
| 700344246H1 | g1109671 | 36 | 17 | gb105pln | Oryza sativa Ca2+ sensitive 3′(2′), 5-diphosphonucleoside 3′(2′) phosphohydrolase | |
| | | | | | mRNA, complete cds. | |
| 700346231H1 | g483586 | 35 | 0 | gb105eukp | ribosomal protein L37 | |
| 700343281H1 | g550019 | 9 | 5 | gb105allp | ribosomal protein L28 | |
| 700344336H1 | g168500 | 75 | −60 | gb105pln | Maize (Zea mays) histone H4 gene (H4C14), complete cds. | |
| 700379941H1 | g166857 | 45 | −37 | gb105pln | Arabidopsis thaliana cytoplasmic ribosomal protein mRNA, complete cds. | |
| 700346011H1 | g1573062 | 18 | 1 | gb105allp | seryl-tRNA synthetase (serS) | |
| 700344283H1 | g313266 | 48 | −46 | gb105pln | T. aestivum gene for phosphoglycerate kinase. | |
| 700380172H1 | g171321 | 29 | −14 | gb105pln | Yeast (Saccharomyces cerevisiae) CRY1 gene, encoding ribosomal protein 59 (rp59), | |
| | | | | | complete cds. | |
| 700344893H1 | g2668742 | 7 | 6 | gb105allp | glycine-rich RNA binding protein | |
| 700380222H1 | g263500 | 5 | 6 | gb105allp | ribosomal protein L10 homolog [Saccharomyces cerevisiae = yeast, Peptide, 236 aa] | |
| 700345645H1 | g295448 | 8 | 3 | gb105eukp | NADPH cytochrome P450 | |
| 700342210H1 | g1177283 | 48 | −19 | gb105eukp | F40F8.10 | |
| 700343917H1 | g415316 | 68 | −46 | gb105pln | Rice mRNA for acidic ribosomal protein P0, complete cds. | |
| 700347040H1 | g2814193 | 26 | −12 | gb105eukp | F39B2.2 | |
| 700342264H1 | g2624416 | 32 | −17 | gb105pln | Zea mays mRNA for ubiquitin carrier protein UBC7. | |
| 700345848H1 | g531057 | 47 | −29 | gb105pln | Wheat mRNA for protein H2B-8, complete cds. | |
| 700281821H2 | g777757 | 73 | −68 | gb105pln | Saccharum hybrid (clone SCUBI561) polyubiquitin mRNA, complete cds. | |
| 700342663H1 | g640006 | 10 | 0 | gb105eukp | J0902 | |
| 700343795H1 | g1016209 | 22 | −13 | gb105eukp | trpG; glutamine amidotransferase associated with anthranilate synthase for tryptophan | |
| | | | | | biosynthesis | |
| 700346958H1 | g836805 | 18 | −0 | gb105eukp | PRE4; proteosome component PRE4; EC 3.4.99.46 | |
| 700346896H1 | g310322 | 16 | −11 | gb105pln | Oryza sativa triosephosphate isomerase (Rictipi2) gene, exons 1-9., | |
| 700346979H1 | g409070 | 18 | 2 | gb105allp | HBp15/L22 | |
| 700343673H1 | g169778 | 15 | 0 | gb105pln | Oryza sativa beta-amylase gene, complete cds. | |
| 700343925H1 | g179102 | 18 | 4 | gb105allp | aspartyl-tRNA synthetase | |
| 700344475H1 | g2244913 | 17 | 6 | gb105eukp | similar to SEN1 protein - yeast; Author-given protein sequence is in conflict with the | |
| | | | | | conceptual translation | |
| 700342329H1 | g456671 | 19 | −1 | gb105pln | T. aestivum VDAC 1 mRNA. | |
| 700342480H1 | g218237 | 20 | −4 | gb105pln | Rice mRNA for ribosomal protein S22 (T47 gene), partial sequence. | |
| 700345326H1 | g497120 | 31 | 1 | gb105eukp | catalyzes the conversion of monodehydroascorbate to ascorbate, oxidizing NADH in | |
| | | | | | the process, binds to flavin as a single subunit; monodehydroascorbate reductase | |
| 700342936H1 | g18259 | 23 | −22 | gb105pln | C. sativus mRNA for cs DnaJ-1. | |
| 700281930H2 | g168500 | 22 | −40 | gb105pln | Maize (Zea mays) histone H4 gene (H4C14), complete cds. | |
| 700344607H1 | g3186 | 12 | −2 | gb105pln | Physarum polycephalum actin gene. | |
| 700380171H1 | g2224915 | 13 | 1 | gb105eukp | beta-expansin | |
| 700343885H1 | g2244829 | 20 | −13 | gb105pln | Arabidopsis thaliana DNA chromosome 4, ESSA I contig fragment No. 2. | |
| 700343031H1 | g886737 | 68 | −55 | gb105pln | Z. mays histone H3 gene. | |
| 700343659H1 | g1747295 | 53 | −39 | gb105pln | Rice mRNA for vacuolar H+-pyrophosphatase, complete cds. | |
| 700343085H1 | g763280 | 44 | −18 | gb105eukp | RNR3; Rnr3p | |
| 700344902H1 | g577530 | 28 | −15 | gb105pln | A. thaliana mRNA for proteasome subunit. | |
| 700380274H1 | g1065506 | 6 | 5 | gb105eukp | C27F2.5 | |
| 700344604H1 | g1477683 | 39 | −31 | gb105pln | Oryza sativa SNF1-related protein kinase (RSk1) mRNA, complete cds. | |
| 700342558H1 | g1877396 | 56 | −49 | gb105pln | R. communis mRNA for shaggy-like kinase, partial. | |
| 700345125H1 | g2842486 | 11 | 6 | gb105eukp | F21O9.120; putative protein | |
| 700342324H1 | g21599 | 13 | 7 | gb105allp | UTP-glucose-1-phosphate uridylyltransferase | |
| 700343718H1 | g1931622 | 22 | −3 | gb105pln | Yeast (Saccharomyces cerevisiae) proteasome cap subunit (PCS1) gene, | |
| | | | | | complete cds. | |
| 700281996H2 | g975888 | 12 | 5 | gb105eukp | myo-inositol-1-phosphate synthase | |
| 700347195H1 | g2599091 | 17 | 12 | gb105pln | Arabidopsis thaliana WD-40 repeat protein MSI4 (MSI4) mRNA, complete cds. | |
| 700343491H1 | g2511530 | 45 | 13 | gb105pln | Eleusine indica alpha tubulin 1 (TUA1) mRNA, complete cds. | |
| 700343115H1 | g289188 | 43 | −7 | gb105eukp | 5′-phosphoribosyl-5-aminoimidazole synthetase; AIR synthetase | |
| 700343596H1 | g168486 | 67 | −46 | gb105pln | Maize glutathione S-transferase gene (GST-I), exon 1. | |
| 700347224H1 | g2302294 | 13 | 6 | gb105allp | unnamed protein product | |
| 700346865H1 | g1841307 | 18 | 5 | gb105pln | Fission Yeast mRNA for ribosomal protein S16 homolog, partial cds. | |
| 700379721H1 | g2626840 | 8 | 5 | gb105allp | CRM1 protein | |
| 700345546H1 | g1276967 | 36 | −13 | gb105eukp | putative ribosomal protein | |
| 700347284H1 | g1232130 | 29 | −6 | gb105eukp | transcription factor HBP-1b(c38) | |
| 700346847H1 | g1488296 | 29 | −11 | gb105pln | Oryza sativa osRAD23 mRNA, complete cds. | |
| 700345439H1 | g20163 | 40 | −24 | gb105pln | O. sativa Rr15 mRNA for 5S ribosomal RNA. | |
| 700343430H1 | g416265 | 23 | −9 | gb105pln | Rice mRNA for ribosomal protein A2, partial sequence. | |
| 700343625H1 | g289153 | 21 | −5 | gb105allp | methylamine oxidase | |
| 700343932H1 | g600768 | 50 | −47 | gb105pln | Oryza sativa cyclophilin 2 (Cyp2) mRNA, complete cds. | |
| 700380178H1 | g2274990 | 35 | −42 | gb105pln | Hordeum vulgare mRNA for expressed sequence tag. | |
| 700345531H1 | g459009 | 14 | −2 | gb105eukp | T20H4.3 | |
| 700342285H1 | g1334511 | 15 | −8 | gb105eukp | COI; cytochrome oxidase subunit 1 | |
| 700345393H1 | g2688821 | 31 | −32 | gb105pln | Prunus armeniaca pyrophosphate-dependent phosphofructo-1-kinase mRNA, | |
| | | | | | partial cds. | |
| 700342414H1 | g2315152 | 30 | −14 | gb105pln | Arabidopsis thaliana mRNA for MAP3K gamma protein kinase. | |
| 700344880H1 | g404406 | 32 | −38 | gb105pln | type 1 actin {type 1} [Emiliania huxleyi = Prymnesiophyte algae, CCMP379, mRNA | |
| | | | | | Partial, 1095 nt]. | |
| 700380409H1 | g1737491 | 27 | −31 | gb105pln | Triticum aestivum poly(A)-binding protein (wheatpab) mRNA, complete cds. | |
| 700346287H1 | g473602 | 41 | −20 | gb105pln | Zea mays W-22 histone H2A mRNA, complete cds. | |
| 700346287H1 | g1325967 | 25 | 3 | gb105pln | T. aestivum histone H2A gene (clone TH274). | |
| 700343334H1 | g2224911 | 14 | −4 | gb105eukp | somatic embryogenesis receptor-like kinase | |
| 700379763H1 | g1911165 | 37 | −17 | gb105pln | S. tuberosum mRNA for soluble-starch-synthase. | |
| 700347059H1 | g2739381 | 27 | −5 | gb105eukp | T9J22.23; putative patatin | |
| 700346579H1 | g189842 | 28 | −10 | gb105allp | prolidase | |
| 700342766H1 | g2827618 | 41 | 10 | gb105pln | Arabidopsis thaliana DNA chromosome 4, BAC clone F10N7 (ESSAII project). | |
| 700346122H1 | g1389639 | 13 | −1 | gb105pln | Pea mRNA for PNDKN1, complete cds. | |
| 700345431H1 | g2773153 | 17 | −30 | gb105pln | Oryza sativa abscisic acid- and stress-inducible protein (Asr1) mRNA, complete cds. | |
| 700342895H1 | g310932 | 44 | −2 | gb105pln | Nicotiana tabacum ribosomal protein L17 mRNA, complete cds. | |
| 700345243H1 | g1044939 | 22 | −12 | gb105pln | Z. mays mRNA for ubiquitin/ribosomal protein S27a fusion protein. | |
| 700345707H1 | g22149 | 70 | −75 | gb105pln | Z. mays mRNA for alpha-tubulin 3. | |
| 700343618H1 | g300082 | 44 | −5 | gb105pln | hsp82 = 82 kda heat shock protein [Zea mays, seedling, leaves, Genomic, 3468 nt]. | |
| 700346123H1 | g1061297 | 17 | 6 | gb105eukp | SPAC24B11.09; unknown | |
| 700342558H1 | g1161511 | 53 | −47 | gb105pln | A. thaliana mRNA for shaggy-like kinase etha. | |
| 700346727H1 | g21794 | 72 | −64 | gb105pln | Wheat histone H4 gene. | |
| 700345170H1 | g500852 | 40 | −33 | gb105pln | Zea mays (clone pAKHSDH2) aspartate kinase-homoserine dehydrogenase mRNA, | |
| | | | | | complete cds. | |
| 700345244H1 | g2160160 | 29 | −13 | gb105eukp | F21M12.5 | |
| 700281928H2 | g1244773 | 19 | −5 | gb105eukp | RPL37A; Rp137ap: 60S ribosomal protein L37a | |
| 700345104H1 | g536891 | 63 | −57 | gb105pln | Wheat mRNA for protein H2A, complete cds, clone wcH2A-4. | |
| 700343725H1 | g1628443 | 21 | −3 | gb105eukp | ORF | |
| 700344290H1 | g2331300 | 60 | −60 | gb105pln | Zea mays ribosomal protein S4 type I (rps4) mRNA, complete cds. | |
| 700347341H1 | g170164 | 29 | −8 | gb105pln | S. guttatum aox1 mRNA encoding an alternative oxidase protein. | |
| 700345562H1 | g625147 | 88 | −78 | gb105pln | Zea mays protein disulfide isomerase (pdi) mRNA, complete cds. | |
| 700344249H1 | g2264320 | 19 | 14 | gb105pln | Arabidopsis thaliana genomic DNA, chromosome 5, P1 clone: MX110, complete | |
| | | | | | sequence. | |
| 700344675H1 | g228 | 15 | −1 | gb105allp | NADH dehydrogenase (ubiquinone) 42 kDa subunit | |
| 700379668H1 | g388600 | 15 | 0 | gb105eukp | F44E2.6 protein | |
| 700346544H1 | g498902 | 33 | −11 | gb105eukp | RPL27-3; ribosomal protein L27 homolog | |
| 700380440H1 | g2735307 | 23 | 6 | gb105allp | voltage dependent anion channel form 3 | |
| 700346351H1 | g2150130 | 10 | 6 | gb105eukp | cytoplasmic ribosomal protein S15a | |
| 700343684H1 | g1122776 | 9 | −1 | gb105eukp | C10C5.3 | |
| 700345376H1 | g2352929 | 31 | −1 | gb105eukp | pCM1; plastidic chorismate mutase 1 | |
| 700346683H1 | g786312 | 22 | −4 | gb105pln | Yeast (Saccharomyces cerevisiae) chromosome SVI cosmid 9705. | |
| 700379913H1 | g2351061 | 28 | 6 | gb105pln | Arabidopsis thaliana genomic DNA, chromosome 5, P1 clone: MAF19. | |
| 700343857H1 | g170901 | 13 | 2 | gb105eukp | peroxisomal membrane protein (PMP20B) | |
| 700281929H2 | g2687424 | 29 | −18 | gb105pln | Helianthus annuus FatB thioesterase (FatB1) mRNA, complete cds. | |
| 700342514H1 | g1710361 | 12 | 8 | gb105allp | splicing factor U2AF65 | |
| 700344863H1 | g2104951 | 10 | 5 | gb105eukp | SDhn-6f; MAP kinase-like protein | |
| 700342963H1 | g167244 | 40 | −30 | gb105pln | Chlorella kessleri elongation factor 2 mRNA, complete cds. | |
| 700344231H1 | g168500 | 52 | −43 | gb105pln | Maize (Zea mays) histone H4 gene (H4C14), complete cds. | |
| 700347011H1 | g296358 | 19 | 6 | gb105eukp | csa; CIT-SAP | |
| 700345902H1 | g1147770 | 20 | −18 | gb105pln | Yeast (Schizosaccharomyces pombe) elongation factor 1-alpha mRNA, complete cds. | |
| 700345142H1 | g1773371 | 15 | −1 | gb105eukp | putative protein disulfide isomerase | |
| 700347224H1 | g17593 | 9 | 6 | gb105allp | THIOREDOXINE | |
| 700342647H1 | g2274983 | 40 | −17 | gb105pln | Hordeum vulgare mRNA for expressed sequence tag. | |
| 700380496H1 | g508723 | 10 | 7 | gb105allp | SAP 61 | |
| 700380428H1 | g1122422 | 67 | −33 | gb105allp | FXR1 gene product | |
| 700343514H1 | g2494115 | 17 | −23 | gb105eukp | T1G11.1 | |
| 700343188H1 | g166970 | 13 | −12 | gb105pln | Hordeum vulgare acyl carrier protein III (Ac13) gene, complete cds. | |
| 700342653H1 | g908756 | 15 | 4 | gb105allp | unknown protein | |
| 700344132H1 | g216457 | 11 | 8 | gb105allp | ORF | |
| 700346945H1 | g798817 | 33 | −20 | gb105pln | A. thaliana mRNA for ribosomal protein L2. | |
| 700343996H1 | g2623679 | 67 | −69 | gb105pln | Zea mays calmodulin (Zmrcalm) mRNA, complete cds. | |
| 700344155H1 | g498902 | 31 | −29 | gb105eukp | RPL27-3; ribosomal protein L27 homolog | |
| 700379831H1 | g20323 | 40 | −43 | gb105pln | O. sativa RAc1 gene for actin. | |
| 700346336H1 | g1518539 | 47 | −35 | gb105pln | Glycine max UDP-glucose dehydrogenase mRNA, complete cds. | |
| 700346092H1 | g32532 | 18 | 0 | gb105allp | ribosomal protein s3 | |
| 700345422H1 | g1149569 | 15 | 3 | gb105eukp | athb-8; HD-zip | |
| 700347118H1 | g168500 | 52 | −27 | gb105pln | Maize (Zea mays) histone H4 gene (H4C14), complete cds. | |
| 700345546H1 | g1669623 | 18 | −4 | gb105eukp | ribosomal protein L39 | |
| 700343223H1 | g1143387 | 21 | −19 | gb105pln | A. thaliana mRNA for Class III ADH. | |
| 700344110H1 | g2443471 | 13 | 11 | gb105pln | Arabidopsis thaliana ASF/SF2 homolog (SF2) gene, complete cds. | |
| 700345264H1 | g2668745 | 32 | −16 | gb105pln | Zea mays inorganic pyrophosphatase (IPP) mRNA, complete cds. | |
| 700342565H1 | g695169 | 35 | −17 | gb105eukp | unknown | |
| 700344133H1 | g168486 | 66 | −65 | gb105pln | Maize glutathione S-transferase gene (GST-I), exon 1. | |
| 700344416H1 | g758292 | 7 | 7 | gb105eukp | YPT53 | |
| 700342570H1 | g1743006 | 26 | −13 | gb105pln | C. paradoxa mRNA for ribosomal protein L13a. | |
| 700344112H1 | g168505 | 56 | −30 | gb105pln | Zea mays histone H3 gene, complete cds. | |
| 700345164H1 | g289707 | 8 | 6 | gb105eukp | K12H4.3 protein | |
| 700379641H1 | g1177601 | 29 | −3 | gb105eukp | bfruct1; hydrplyze sucrose into fructose and glucose; invertase; EC 3.2.1.26 | |
| 700347167H1 | g2511530 | 38 | −45 | gb105pln | Eleusine indica alpha tubulin 1 (TUA1) mRNA, complete cds. | |
| 700346975H1 | g337453 | 7 | 7 | gb105allp | hnRNP B1 protein | |
| 700346033H1 | g4099 | 29 | −13 | gb105eukp | PAP1; poly(A) polymerase | |
| 700342745H1 | g1809261 | 20 | −9 | gb105eukp | Pto; serine/threonine protein kinase Pto | |
| 700344690H1 | g2281098 | 21 | −8 | gb105eukp | F18O19.17 | |
| 700344086H1 | g1843628 | 31 | −7 | gb105eukp | bsh; SNF5 homolog BSH | |
| 700344366H1 | g2739218 | 45 | −32 | gb105pln | Hordeum vulgare S28 ribosomal protein. | |
| 700342705H1 | g2366749 | 26 | −20 | gb105pln | Nicotiana sylvestris mRNA for RNA binding protein, complete cds. | |
| 700380062H1 | g886739 | 42 | −10 | gb105pln | Z. mays histone H4 gene. | |
| 700379721H1 | g1323393 | 6 | 7 | gb105eukp | CRM1 | |
| 700345940H1 | g1724113 | 25 | −6 | gb105pln | Avena fatua Af10-protein mRNA, complete cds. | |
| 700343030H1 | g2827001 | 32 | −29 | gb105pln | Triticum aestivum 70 dKa heat shock protein (TaHSP70d) mRNA, complete cds. | |
| 700342461H1 | g1001579 | 12 | 2 | gb105allp | ABC1-like | |
| 700346902H1 | g416265 | 27 | −10 | gb105pln | Rice mRNA for ribosomal protein A2, partial sequence. | |
| 700343391H1 | g2331130 | 30 | −19 | gb105pln | Oryza sativa glycine-rich protein (SGRP1) mRNA, complete cds. | |
| 700346888H1 | g248338 | 66 | −34 | gb105pln | polyubiquitin [maize, Genomic, 3439 nt]. | |
| 700343852H1 | g1015632 | 13 | 1 | gb105eukp | SUI2 | |
| 700343977H1 | g1495250 | 34 | −25 | gb105pln | A. thaliana mRNA for heat-shock protein. | |
| 700380670H1 | g644491 | 58 | −59 | gb105pln | Corn mRNA for elongation factor 1A. | |
| 700346956H1 | g2811223 | 16 | −15 | gb105pln | Arabidopsis thaliana fimbrin 1 mRNA, partial cds. | |
| 700344960H1 | g168500 | 85 | −76 | gb105pln | Maize (Zea mays) histone H4 gene (H4C14), complete cds. | |
| 700346978H1 | g1066162 | 42 | −32 | gb105pln | B. napus mRNA for glyoxysomal beta-keotacyl-thiolase precursor. | |
| 700347305H1 | g168426 | 45 | −21 | gb105eukp | bt1; brittle-1 protein | |
| 700343386H1 | g1653962 | 15 | 3 | gb105allp | hypothetical protein | |
| 700342545H1 | g2351687 | 18 | −5 | gb105eukp | ap51-2; cytoadherence to epithelial cell surface; adhesin protein AP51-2 | |
| 700344402H1 | g1762945 | 21 | −9 | gb105eukp | ORF; able to induce HR-like lesions | |
| 700347130H1 | g471319 | 40 | 2 | gb105eukp | HvB12D | |
| 700343593H1 | g30377 | 30 | −13 | gb105allp | cytokeratin 13 | |
| 700345212H1 | g22312 | 53 | −59 | gb105pln | Maize ABA-inducible gene for glycine-rich protein (ABA = abscisic acid). | |
| 700346686H1 | g1183936 | 16 | 2 | gb105pln | P. sativum 5S rRNA gene. | |
| 700345654H1 | g429006 | 19 | 14 | gb105pln | Rice mRNA for MCM3 (gene name SS300), partial cds. | |
| 700346681H1 | g1667593 | 24 | 3 | gb105pln | Oryza sativa transmembrane protein mRNA, complete cds. | |
| 700346342H1 | g163030 | 13 | 4 | gb105allp | endonexin | |
| 700343210H1 | g2529657 | 31 | −23 | gb105pln | Arabidopsis thaliana chromosome II BAC T30B22 genomic sequence, complete | |
| | | | | | sequence. | |
| 700345559H1 | g1930071 | 41 | −25 | gb105pln | Oryza sativa thioredoxin h mRNA, complete cds. | |
| 700342466H1 | g2232174 | 35 | −10 | gb105allp | MLD | |
| 700345930H1 | g2570104 | 26 | −6 | gb105pln | S. latifolia mRNA, clone CCLS 30.3. | |
| 700345268H1 | g4366 | 8 | 7 | gb105eukp | rp 28 | |
| 700343168H1 | g599992 | 10 | −0 | gb105eukp | KGD1; 2-oxoglutarate dehydrogenase E1 component | |
| 700343606H1 | g21800 | 29 | −32 | gb105pln | T. asestivum L mRNA for histone H2B. | |
| 700344026H1 | g960356 | 67 | −59 | gb105pln | Barley mRNA for S-adenosylmethionine synthetase, complete cds. | |
| 700347376H1 | g472343 | 20 | 1 | gb105eukp | <beta>Cop | |
| 700343432H1 | g297888 | 39 | −29 | gb105pln | D. carota mRNA for B2 protein. | |
| 700345251H1 | g1272407 | 27 | −7 | gb105pln | Arabidopsis thaliana immunophilin (FKBP15-2) mRNA, complete cds. | |
| 700380662H1 | g2832611 | 27 | −32 | gb105pln | Arabidopsis thaliana DNA chromosome 4, BAC clone F13C5 (ESSAII project). | |
| 700379791H1 | g167072 | 43 | −37 | gb105pln | Barley ubiquitin (mub1) gene, complete cds. | |
| 700347095H1 | g1907231 | 8 | 8 | gb105allp | YCR093w, len:2108 | |
| 700346185H1 | g2317876 | 41 | −2 | gb105eukp | cysteine protease inhibitor | |
| 700343888H1 | g544501 | 11 | −4 | gb105eukp | SEC13; Sec13p | |
| 700346763H1 | g600768 | 56 | −47 | gb105pln | Oryza sativa cyclophilin 2 (Cyp2) mRNA, complete cds. | |
| 700345519H1 | g2196547 | 23 | −2 | gb105pln | Nicotiana tabacum DNA-binding protein (T231) mRNA, complete cds. | |
| 700345413H1 | g393400 | 90 | −80 | gb105pln | Z. mays mRNA for alpha-tubulin. | |
| 700380406H1 | g170746 | 54 | −46 | gb105pln | Wheat histone H4 TH091 gene, complete cds. | |
| 700345651H1 | g2642159 | 28 | −8 | gb105eukp | T5I7.7; putative mannose-1-phosphate guanyltransferase | |
| 700342292H1 | g2662346 | 59 | −46 | gb105pln | Oryza sativa mRNA for EF-1 alpha, complete cds. | |
| 700342244H1 | g1279763 | 26 | 4 | gb105allp | arfaptin 2 | |
| 700342485H1 | g2459406 | 40 | −28 | gb105pln | Arabidopsis thaliana chromosome II BAC F4P9 genomic sequence, complete | |
| | | | | | sequence. | |
| 700379705H1 | g168689 | 13 | 14 | gb105pln | Maize zein (zE25) pseudogene, 5′ end. | |
| 700344441H1 | g18891 | 8 | −2 | gb105eukp | aldose reductase-related protein | |
| 700342526H1 | g1360212 | 9 | 4 | gb105eukp | ORF YLL029w | |
| 700344063H1 | g2570506 | 45 | −36 | gb105pln | Oryza sativa ribosomal protein mRNA, complete cds. | |
| 700346689H1 | g2264305 | 24 | 1 | gb105pln | Arabidopsis thaliana genomic DNA, chromosome 5, P1 clone: MBK23, complete | |
| | | | | | sequence. | |
| 700345527H1 | g170746 | 69 | −57 | gb105pln | Wheat histone H4 TH091 gene, complete cds. | |
| 700342370H1 | g168492 | 60 | −48 | gb105pln | Corn histone H3 (H3C3) gene, complete cds. | |
| 700345458H1 | g168500 | 61 | −45 | gb105pln | Maize (Zea mays) histone H4 gene (H4C14), complete cds. | |
| 700343034H1 | g416460 | 10 | 8 | gb105allp | unidentified protein | |
| 700345744H1 | g168498 | 79 | −79 | gb105pln | Corn histone H4 (H4C13) gene, complete cds. | |
| 700343108H1 | g643073 | 53 | −32 | gb105pln | Fragaria x ananassa putative 40S ribosomal protein s12 mRNA, complete cds. | |
| 700346735H1 | g602605 | 75 | −75 | gb105pln | Zea mays Tandem genes for alpha1-tubulin and alpha2-tubulin. | |
| 700345476H1 | g732949 | 22 | −6 | gb105eukp | SSM1a | |
| 700379682H1 | g861204 | 22 | −12 | gb105pln | Chlamydomonas reinhardtii ADP-ribosylation factor (ARF) mRNA, complete cds. | |
| 700347191H1 | g1122803 | 11 | 7 | gb105allp | F49C12.8 | |
| 700343201H1 | g450300 | 28 | −17 | gb105eukp | receptor type serine/threonine kinase; protein kinase; OSPK10 | |
| 700343942H1 | g168500 | 75 | −60 | gb105pln | Maize (Zea mays) histone H4 gene (H4C14), complete cds. | |
| 700346225H1 | g456519 | 22 | −13 | gb105eukp | ORF203 | |
| 700342403H1 | g2293275 | 8 | 1 | gb105allp | YtaG | |
| 700342352H1 | g1575469 | 25 | −18 | gb105eukp | S19; ribosomal protein S19 | |
| 700342229H1 | g1519248 | 35 | −20 | gb105pln | Oryza sativa GF14-b protein mRNA, complete cds. | |
| 700345188H1 | g1770190 | 14 | 4 | gb105eukp | cycD1; cyclin-D like protein | |
| 700346724H1 | g1946371 | 11 | 2 | gb105eukp | T06B20.17 | |
| 700345930H1 | g2738759 | 19 | −5 | gb105pln | Arabidopsis thaliana 5′-adenylylsulfate reductase (APR3) gene, complete cds. | |
| 700344254H1 | g143411 | 9 | −8 | gb105allp | ORF1 | |
| 700379964H1 | g1778051 | 22 | 8 | gb105allp | Prt1 homolog | |
| 700345647H1 | g262260 | 19 | 8 | gb105eukp | Rbp3 | |
| 700346346H1 | g2104523 | 13 | 16 | gb105pln | Arabidopsis thaliana chromosome IV BAC clone T10M13, complete sequence. | |
| 700346294H1 | g429015 | 26 | −35 | gb105pln | Rice mRNA for ribosomal protein (gene name SS498), partial cds. | |
| 700343175H1 | g2244996 | 11 | 1 | gb105eukp | similarity to a membrane-associated salt-inducible protein | |
| 700343945H1 | g2266661 | 42 | −24 | gb105pln | Hordeum vulgare mRNA for 14-3-3 protein (Hv1433c). | |
| 700344331H1 | g1304265 | 30 | −6 | gb105pln | Wheat mRNA for HALF-1, complete cds. | |
| 700380115H1 | g2511588 | 18 | −5 | gb105eukp | prc1; multicatalytic endopeptidase complex, proteasome component, alpha subunit; | |
| | | | | | EC 3.4.99.46 | |
| 700346718H1 | g602252 | 93 | −87 | gb105pln | Zea mays enolase (eno2) mRNA, complete cds. | |
| 700342376H1 | g182483 | 62 | −34 | gb105allp | prefibroblast collagenase inhibitor | |
| 700342661H1 | g1666172 | 32 | −14 | gb105pln | N. plumbaginifolia mRNA for BTF3-like transcription factor. | |
| 700345355H1 | g1815680 | 26 | −22 | gb105pln | Oryza sativa expansin (OS-EXP4) mRNA, complete cds. | |
| 700346313H1 | g1122433 | 8 | 6 | gb105allp | polypyrimidine tract-binding protein | |
| 700346252H1 | g790507 | 57 | −72 | gb105pln | Z. mays mRNA for 60S acidic ribosomal protein. | |
| 700345314H1 | g168500 | 49 | −68 | gb105pln | Maize (Zea mays) histone H4 gene (H4C14), complete cds. | |
| 700346857H1 | g2245113 | 31 | −11 | gb105eukp | glycerol-3-phosphate permease homolog | |
| 700347265H1 | g2431766 | 86 | −78 | gb105pln | Zea mays acidic ribosomal protein P3a (rpp3a) mRNA, complete cds. | |
| 700346341H1 | g531481 | 14 | −15 | gb105pln | P. ciliare (Higgins) apospory associated mRNA, 1398bp. | |
| 700343525H1 | g1899026 | 29 | −54 | gb105pln | Zea mays superoxide dismutase 4A (sod4A) gene, complete cds. | |
| 700342580H1 | g397395 | 57 | −38 | gb105pln | Z. mays MNB1b mRNA for DNA-binding protein. | |
| 700380484H1 | g1737491 | 20 | 7 | gb105pln | Triticum asestivum poly(A)-binding protein (wheatpab) mRNA, complete cds. | |
| 700379842H1 | g1167955 | 20 | −22 | gb105eukp | putative 32.7 kDa jasmonate-induced protein | |
| 700343820H1 | g510209 | 7 | 5 | gb105allp | elongation factor 1 | |
| 700380233H1 | g2264306 | 34 | −27 | gb105pln | Arabidopsis thaliana genomic DNA, chromosome 5, P1 clone: MBK5, complete | |
| | | | | | sequence. | |
| 700346792H1 | g218233 | 27 | −3 | gb105pln | Rice mRNA for NDP kinase (T164 gene), partial sequence. | |
| 700346610H1 | g2431766 | 55 | −54 | gb105pln | Zea mays acidic ribosomal protein P3a (rpp3a) mRNA, complete cds. | |
| 700343526H1 | g1514643 | 20 | −2 | gb105eukp | multidrug resistance protein; PDR5-like ABC transporter | |
| 700345209H1 | g460990 | 61 | −56 | gb105pln | O. sativa (Arborio) Beta Tubulin mRNA, clone OSTB-16. | |
| 700346062H1 | g2564046 | 19 | −6 | gb105pln | Arabidopsis thaliana genomic DNA, chromosome 5, P1 clone: MGI19, complete | |
| | | | | | sequence. | |
| 700342372H1 | g168494 | 62 | −54 | gb105pln | Maize (Zea mays) histone H3 gene (H3C2), complete cds. | |
| 700281841H2 | g1556445 | 66 | −67 | gb105pln | Hordeum vulgare, alpha tubulin (tubA) mRNA, complete cds. | |
| 700345622H1 | g940843 | 17 | −4 | gb105eukp | orf 06116 | |
| 700342524H1 | g169295 | 16 | −2 | gb105pln | Pharbitis nil heat shock protein 83 (Hsp83) gene, complete cds. | |
| 700343953H1 | g2274990 | 18 | 12 | gb105pln | Hordeum vulgare mRNA for expressed sequence tag. | |
| 700342706H1 | g166859 | 37 | −43 | gb105pln | Arabidopsis thaliana ribosomal protein gene, complete cds. | |
| 700344059H1 | g1890156 | 11 | 3 | gb105eukp | ATGBST1; gluthatione S-transferase | |
| 700380401H1 | g2654209 | 27 | −13 | gb105pln | Spinacia oleracea heat shock 70 protein (HSC70-10) mRNA, nuclear gene encoding | |
| | | | | | mitochondrial protein, complete cds. | |
| 700346181H1 | g886739 | 47 | 13 | gb105pln | Z. mays histone H4 gene. | |
| 700345843H1 | g2244915 | 10 | 4 | gb105allp | strong homology to reverse transcriptase | |
| 700345644H1 | g22275 | 88 | −72 | gb105pln | Maize mRNA for ferritin (clone FM1). | |
| 700343318H1 | g1703631 | 18 | −3 | gb105eukp | C24A11.9 | |
| 700346558H1 | g488531 | 16 | −8 | gb105eukp | anc2; cytosolic chaperonin; Anc2p | |
| 700342726H1 | g2662048 | 8 | 3 | gb105eukp | BcRK1; receptor kinase 1 | |
| 700343991H1 | g2264302 | 10 | 17 | gb105pln | Arabidopsis thaliana genomic DNA, chromosome 5, P1 clone: MAC12, complete | |
| | | | | | sequence. | |
| 700344504H1 | g2668743 | 37 | −38 | gb105pln | Zea mays ubiquitin conjugating enzyme (UBC) mRNA, complete cds. | |
| 700346970H1 | g2464945 | 28 | −13 | gb105eukp | beta-galactosidase | |
| 700345104H1 | g1154953 | 42 | −49 | gb105pln | T. aestivum histone H2A gene. | |
| 700346414H1 | g2230878 | 8 | 7 | gb105allp | hNop56 | |
| 700380791H1 | g829024 | 21 | 4 | gb105allp | ATPase | |
| 700344441H1 | g1835701 | 26 | −20 | gb105eukp | m6pr; NADPH-dependent mannose 6-phosphate reductase; EC 1.1.1.224 | |
| 700344944H1 | g2224913 | 22 | −4 | gb105eukp | beta-expansin | |
| 700379731H1 | g169295 | 28 | −15 | gb105pln | Pharbitis nil heat shock protein 83 (Hsp83) gene, complete cds. | |
| 700343777H1 | g2244829 | 13 | 13 | gb105pln | Arabidopsis thaliana DNA chromosome 4, ESSA I contig fragment No. 2. | |
| 700344852H1 | g577818 | 55 | −59 | gb105pln | Z. mays gene for H2B histone (gH2B4). | |
| 700347018H1 | g1419090 | 45 | −18 | gb105eukp | 37kDa chloroplast inner envelope membrane polypeptide precursor | |
| 700343511H1 | g1931644 | 17 | −7 | gb105eukp | T19D16.10; membrane protein PTM1 precursor isolog | |
| 700342211H1 | g297015 | 15 | −7 | gb105eukp | fructokinase | |
| 700380788H1 | g248338 | 59 | −44 | gb105pln | polyubiquitin [maize, Genomic, 3439 nt]. | |
| 700345702H1 | g1843627 | 20 | −10 | gb105pln | Arabidopsis thaliana SNF5 homolog BSH (bsh) mRNA, complete cds. | |
| 700345537H1 | g575684 | 15 | −0 | gb105eukp | AMD1 | |
| 700345465H1 | g633135 | 33 | −29 | gb105pln | Zostera marina mRNA for putative plasma membrane H+-ATPase, complete cds. | |
| 700343529H1 | g2160162 | 22 | −1 | gb105eukp | F21M12.8 | |
| 700342532H1 | g1771562 | 6 | 5 | gb105allp | neuronal potassium channel | |
| 700346920H1 | g296703 | 32 | −18 | gb105pln | Yeast (Saccharomyces pombe) FIB gene encoding fibrillarin. | |
| 700281987H2 | g1747418 | 30 | −6 | gb105pln | Z. mays mRNA for eukaryotic initiation factor-5. | |
| 700344425H1 | g868263 | 16 | 6 | gb105allp | similar to C. elegans lin-19 | |
| 700347009H1 | g2253010 | 17 | 3 | gb105allp | MAP3K delta-1 protein kinase | |
| 700345458H1 | g21794 | 63 | −45 | gb105pln | Wheat histone H4 gene. | |
| 700342318H1 | g2599091 | 23 | −1 | gb105pln | arabidopsis thaliana WD-40 repeat protein MSI4 (MSI4) mRNA, complete cds. | |
| 700343056H1 | g464145 | 8 | −6 | gb105eukp | beta-amylase; EC 3.2.1.2 | |
| 700343807H1 | g945086 | 44 | −37 | gb105pln | Glycine max transcription factor TFIIB mRNA, complete cds. | |
| 700344010H1 | g577824 | 16 | 9 | gb105pln | Z. mays gene for H2B histone (gH2B3). | |
| 700344759H1 | g2252831 | 8 | −0 | gb105eukp | A_IG005I10.9 | |
| 700345625H1 | g668986 | 52 | −39 | gb105pln | S. tuberosum TYKY2 mRNA for NADH:ubiquinone oxidoreductase. | |
| 700344859H1 | g2463196 | 13 | 5 | gb105allp | MAD2 | |
| 700344979H1 | g710549 | 11 | 4 | gb105allp | ankyrin 3 | |
| 700344094H1 | g474321 | 22 | −7 | gb105pln | Yeast ste13+ gene for RNA helicase, complete cds. | |
| 700380411H1 | g2583081 | 5 | 7 | gb105allp | microsomal glutathione S-transferase 3 | |
| 700342709H1 | g2342495 | 11 | 12 | gb105pln | Ananas comosus mRNA for bromelain, complete cds. | |
| 700347138H1 | g602605 | 76 | −64 | gb105pln | Zea mays tandem genes for alpha1-tubulin and alpha2-tubulin. | |
| 700343863H1 | g429009 | 41 | −3 | gb105pln | Rice mRNA for LMW heat shock protein (gene name SS386), partial cds. | |
| 700343167H1 | g642800 | 14 | −6 | gb105eukp | PTC1; Ptc1p | |
| 700346919H1 | g1938574 | 32 | −17 | gb105eukp | B0025.2 | |
| 700343023H1 | g2811225 | 24 | −1 | gb105pln | Arabidopsis thaliana fimbrin 2 mRNA, complete cds. | |
| 700344826H1 | g20681 | 12 | 7 | gb105allp | 508 aa peptide | |
| 700345075H1 | g2827524 | 51 | −26 | gb105eukp | F8F16.110; predicted protein | |
| 700345277H1 | g170434 | 40 | −36 | gb105pln | Tomato ATP-dependent protease (CD4B) gene, complete cds. | |
| 700347071H1 | g600768 | 39 | −27 | gb105pln | Oryza sativa cyclophilin 2 (Cyp2) mRNA, complete cds. | |
| 700345647H1 | g1781299 | 23 | −1 | gb105eukp | splicing factgor; transformer-SR ribonucleoprotein | |
| 700342640H1 | g157943 | 43 | −19 | gb105eukp | Mhc | |
| 700342384H1 | g550021 | 20 | 0 | gb105allp | ribosomal protein S5 | |
| 700343648H1 | g287628 | 42 | −5 | gb105eukp | YS29A; ribosomal protein YS29 | |
| 700344636H1 | g22302 | 85 | −1 | gb105pln | Maize Gpc1 gene for glyceraldehyde-3-phosphate dehydrogenase (GADPH) subunit C. | |
| 700346975H1 | g337449 | 7 | 7 | gb105allp | hnRNP A2 protein | |
| 700346745H1 | g1658197 | 13 | 6 | gb105eukp | calreticulin | |
| 700345824H1 | g599623 | 31 | −31 | gb105pln | A. thaliana Aco gene. | |
| 700345103H1 | g2724049 | 22 | −12 | gb105pln | Fritillaria agrestis clone CA1 glutaredoxin type I (glr1) mRNA, complete cds. | |
| 700346010H1 | g1519359 | 29 | −13 | gb105eukp | RP49; ribosomal protein 49 | |
| 700342494H1 | g755165 | 12 | 3 | gb105allp | ClpP | |
| 700345085H1 | g20187 | 75 | −68 | gb105pln | O. sativa gene encoding calmodulin. | |
| 700343014H1 | g2832633 | 26 | −11 | gb105eukp | F13C5.220; putative protein | |
| 700345334H1 | g1498052 | 25 | 3 | gb105pln | Zea mays ribosomal protein S8 mRNA, complete cds. | |
| 700343577H1 | g7286954 | 36 | −18 | gb105eukp | DNA helicase; DNA helicase type protein | |
| 700343502H1 | g1370147 | 21 | −18 | gb105pln | L. japonicus mRNA for small GTP-binding protein, RAB11D. | |
| 700347070H1 | g1865682 | 19 | −1 | gb105pln | A. thaliana 16 kb chromosome 1 DNA fragment. | |
| 700345433H1 | g531055 | 41 | −19 | gb105pln | Wheat mRNA for protein H2B-6, complete cds. | |
| 700379651H1 | g2431768 | 48 | −38 | gb105pln | Zea mays acidic ribosomal protein P1a (rpp1a) mRNA, complete cds. | |
| 700346058H1 | g416265 | 28 | −14 | gb105pln | Rice mRNA for ribosomal protein A2, partial sequence. | |
| 700344144H1 | g466442 | 28 | −6 | gb105pln | Arabidopsis thaliana protein phosphatase mRNA, complete cds. | |
| 700380144H1 | g1814400 | 43 | −33 | gb105pln | Mesembryanthemum crystallinum phosphoglucomutase (PGM) mRNA, complete cds. | |
| 700344872H1 | g1814402 | 44 | −50 | gb105pln | Mesembryanthemum crystallinum methionine synthase (MetE) mRNA, complete cds. | |
| 700346645H1 | g1469041 | 18 | −7 | gb105eukp | F23B12.5 | |
| 700345657H1 | g19700 | 54 | −16 | gb105pln | N. plumbaginifolia mRNA NeIF-5A1 for initiation factor 5A(1). | |
| 700342234H1 | g2160167 | 18 | −3 | gb105eukp | F21M12.15 | |
| 700343835H1 | g2388580 | 11 | 3 | gb105eukp | YUP8H12.22 | |
| 700342691H1 | g22314 | 92 | −76 | gb105pln | Maize mRNA for GSH gluthathione S-transferase I (GST; EC 2.5.1.18). | |
| 700380638H1 | g1143387 | 31 | −15 | gb105pln | A. thaliana mRNA for Class III ADH. | |
| 700343128H1 | g473602 | 32 | −14 | gb105pln | Zea mays W-22 histone H2A mRNA, complete cds. | |
| 700343330H1 | g2425065 | 94 | −76 | gb105pln | Zea mays cysteine proteinase Mir3 (mir3) mRNA, complete cds. | |
| 700342570H1 | g1301950 | 19 | 0 | gb105pln | Yeast (Saccharomyces cerevisiae) chromosome XIV reading frame ORF YNL069c. | |
| 700343107H1 | g2736287 | 39 | −18 | gb105pln | Camptotheca acuminata isopentenyl diphosphate isomerase II (IPI2) mRNA, | |
| | | | | | complete cds. | |
| 700342328H1 | g474167 | 47 | −35 | gb105pln | N. tabacum mRNA for phosphoglyceromutase. | |
| 700379938H1 | g313026 | 41 | −27 | gb105pln | L. esculentum rp138 mRNA for ribosomal protein L38. | |
| 700345774H1 | g2160158 | 32 | −15 | gb105eukp | F21M12.3 | |
| 700346048H1 | g1145627 | 19 | −7 | gb105eukp | lipase | |
| 700345515H1 | g2191135 | 13 | −5 | gb105eukp | A_IG002N01.14 | |
| 700344982H1 | g393077 | 8 | 1 | gb105eukp | WHI3; size control; possible RNA binding protein; Whi3p | |
| 700344150H1 | g1060893 | 51 | −1 | gb105allp | human CIRP | |
| 700345506H1 | g695310 | 13 | 14 | gb105pln | Cucumis sativus glyoxysomal malate dehydrogenase (mdhG) mRNA, complete cds. | |
| 700343918H1 | g625147 | 100 | −88 | gb105pln | Zea mays protein disulfide isomerase (pdi) mRNA, complete cds. | |
| 700342936H1 | g1872162 | 22 | −23 | gb105pln | Arabidopsis thaliana DnaJ homolog (agj) mRNA, complete cds. | |
| 700345202H1 | g2760324 | 21 | −4 | gb105eukp | F1N21.9 | |
| 700345963H1 | g2564051 | 28 | −6 | gb105pln | Arabidopsis thaliana genomic DNA, chromosome 5, P1 clone: MWD9, complete | |
| | | | | | sequence. | |
| 700342476H1 | g22324 | 32 | −25 | gb105pln | Z. mays mRNA for H2B histone (clone cH2B221). | |
| 700347158H1 | g1622720 | 32 | −10 | gb105pln | Kalanchoe daigremontiana V-type H+-ATPase 16 kDa subunit mRNA, complete cds. | |
| 700345556H1 | g297172 | 6 | 6 | gb105allp | ribosomal protein S7 | |
| 700346378H1 | g2370231 | 27 | −17 | gb105pln | Hordeum vulgare mRNA for putative acyl-CoA oxidase. | |
| 700345286H1 | g2267592 | 42 | −33 | gb105pln | Oryza sativa glycine-rich RNA-binding protein mRNA, complete cds. | |
| 700345682H1 | g169295 | 36 | 3 | gb105pln | Pharbitis nil heat shock protein 83 (Hsp83) gene, complete cds. | |
| 700344486H1 | g1001440 | 7 | 7 | gb105allp | beta transducin-like protein | |
| 700380745H1 | g587566 | 16 | 0 | gb105eukp | MPP; mitochondrial processing peptidase | |
| 700346443H1 | g19101 | 39 | −22 | gb105pln | H. vulgare mRNA for ribosomal protein L17-1. | |
| 700343021H1 | g596195 | 16 | −2 | gb105eukp | actin | |
| 700342459H1 | g644491 | 74 | −66 | gb105pln | Corn mRNA for elongation factor 1A. | |
| 700346688H1 | g556559 | 74 | −57 | gb105pln | Rice mRNA for homologue of Tat binding protein, complete cds. | |
| 700346226H1 | g2738948 | 12 | −12 | gb105pln | Fragaria x ananassa cytosolic ascorbate peroxidase (APX-c) mRNA, complete cds. | |
| 700346012H1 | g536250 | 26 | −3 | gb105eukp | HMT1 | |
| 700343291H1 | g1890281 | 13 | 4 | gb105eukp | transformation-sensitive protein homolog | |
| 700342414H1 | g2828185 | 16 | −7 | gb105pln | Arabidopsis thaliana genomic DNA, chromosome 5, P1 clone: MUD21, complete | |
| | | | | | sequence. | |
| 700345694H1 | g1619841 | 22 | −3 | gb105eukp | rab1-like | |
| 700344601H1 | g1408306 | 26 | −6 | gb105eukp | qinA; QinA | |
| 700345889H1 | g473976 | 69 | −56 | gb105pln | Rice mRNA, partial homologous to elongation factor 1-alpha gene. | |
| 700345685H1 | g2565006 | 12 | 4 | gb105eukp | T3F12.7; putative sulfate transporter | |
| 700342967H1 | g169792 | 63 | −55 | gb105pln | Rice histone 3 gene, complete cds. | |
| 700380168H1 | g2239257 | 67 | −55 | gb105pln | Zea mays mRNA for cinnamyl alcohol dehydrogense. | |
| 700379996H1 | g298097 | 15 | 4 | gb105allp | subunit of coatomer complex | |
| 700346106H1 | g20163 | 37 | −20 | gb105pln | O. sativa Rr15 mRNA for 5S ribosomal RNA. | |
| 700343165H1 | g2213557 | 12 | 2 | gb105allp | hypothetical protein | |
| 700346388H1 | g1185553 | 17 | 10 | gb105pln | Zea mays glyceraldehyde-3-phosphate dehydrogenase (gpc2) gene, complete cds. | |
| 700345474H1 | g20255 | 60 | −44 | gb105pln | O. sativa gene for heat shock protein 82 HSP82. | |
| 700343084H1 | g927642 | 33 | −36 | gb105pln | Hordeum vulgare mRNA for betaine aldehyde dehydrogenase, complete cds. | |
| 700345959H1 | g1841501 | 68 | −69 | gb105pln | Z. mays mRNA for glutathione-dependent formaldehyde dehydrogenase. | |
| 700344088H1 | g1314048 | 57 | −48 | gb105pln | O. sativa mRNA for archain/delta-COP. | |
| 700347280H1 | g1906829 | 27 | −28 | gb105pln | A. thaliana hsp88.1 gene. | |
| 700343115H1 | g945060 | 42 | −7 | gb105eukp | pur5; aminoimidazole ribonucleotide (AIRS) synthetase; EC 6.3.3.1 | |
| 700342785H1 | g511151 | 36 | 11 | gb105pln | S. tuberosum gene for 25S ribosomal RNA (3′ end). | |
| 700345346H1 | g2275004 | 20 | 0 | gb105pln | Hordeum vulgare mRNA for expressed sequence tag. | |
| 700347066H1 | g1737218 | 19 | 5 | gb105eukp | vacuolar sorting receptor homolog | |
| 700342366H1 | g1419367 | 19 | 1 | gb105pln | Z. mays ZmABP2 mRNA for actin depolymerizing factor. | |
| 700347114H1 | g20255 | 46 | −16 | gb105pln | O. sativa gene for heat shock protein 82 HSP82. | |
| 700346309H1 | g1184771 | 52 | −56 | gb105pln | Zea mays glyceraldehyde-3-phosphate dehydrogenase GAPC2 (gpc2) mRNA, | |
| | | | | | complete cds. | |
| 700345620H1 | g2623021 | 5 | 7 | gb105allp | 2-phosphoglycerate kinase | |
| 700342528H1 | g2425065 | 23 | −1 | gb105pln | Zea mays cysteine proteinase Mir3 (mir3) mRNA, complete cds. | |
| 700281956H2 | g1498052 | 34 | −34 | gb105pln | Zea mays ribosomal protein S8 mRNA, complete cds. | |
| 700342712H1 | g1431621 | 40 | −52 | gb105pln | T. repens mRNA for protein kinase. | |
| 700343266H1 | g1658196 | 16 | 15 | gb105pln | Ricinus communis calreticulin mRNA, complete cds. | |
| 700343142H1 | g2766712 | 26 | −4 | gb105pln | Arabidopsis thaliana ecotype K1-0 chromomethylase (CMT1) mRNA, complete cds. | |
| 700342345H1 | g433043 | 21 | −21 | gb105pln | Zea mays W-22 clone PREM-1E retroelement PREM-1, partial sequence. | |
| 700343654H1 | g2632411 | 12 | 4 | gb105allp | ribosomal protein L17 (BL15) | |
| 700347195H1 | g2599092 | 24 | 2 | gb105eukp | MSI4; WD-40 repeat protein MSI4 | |
| 700342655H1 | g2257743 | 34 | −6 | gb105eukp | lysine-sensitive aspartate kinase | |
| 700344041H1 | g2435515 | 10 | 4 | gb105eukp | A_TM017A05.3 | |
| 700343031H1 | g168492 | 69 | −56 | gb105pln | Corn histone H3 (H3C3) gene, complete cds. | |
| 700380515H1 | g2766529 | 6 | 7 | gb105allp | inositol polyphosphate 5-phosphatase II | |
| 700380563H1 | g1321660 | 28 | −36 | gb105pln | Rice mRNA for ascorbate peroxidase, complete cds. | |
| 700342977H1 | g288062 | 29 | −16 | gb105pln | A. thaliana mRNA for ketol-acid reductoisomerase subunit. | |
| 700347334H1 | g1905929 | 23 | −22 | gb105pln | Oryza sativa MADS box protein OsMADS45 mRNA, complete cds. | |
| 700344050H1 | g496163 | 92 | −61 | gb105pln | Zea mays ribosome-inactivating protein (RIP2) gene, complete cds. | |
| 700379658H1 | g2264307 | 16 | −5 | gb105pln | Arabidopsis thaliana genomic DNA, chromosome 5, P1 clone: MED24, complete | |
| | | | | | sequence. | |
| 700347156H1 | g625108 | 49 | −8 | gb150eukp | YLR388W; Ylr388wp | |
| 700343430H1 | g790507 | 68 | −78 | gb150pln | Z. mays mRNA for 60S acidic ribosomal protein. | |
| 700380062H1 | g170746 | 40 | −11 | gb105pln | Wheat histone H4 TH091 gene, complete cds. | |
| 700343177H1 | g2335089 | 20 | −2 | gb105pln | Arabidopsis thaliana chromosome II BAC T11A07 genomic sequence, complete | |
| | | | | | sequence. | |
| 700346817H1 | g2570500 | 42 | −29 | gb105pln | Oryza sativa inorganic pyrophosphtase (Ipp) mRNA, complete cds. | |
| 700379734H1 | g1173622 | 11 | 6 | gb105allp | homeobox protein | |
| 700281875H2 | g1136574 | 47 | −43 | gb105pln | Sorghum bicolor heat shock protein 70 (hsp70) pseudogene. | |
| 700344316H1 | g940843 | 14 | −2 | gb105eukp | orf 06116 | |
| 700380535H1 | g218088 | 51 | −51 | gb105pln | Rice mRNA for ribosomal protein 117 (249 gene), partial sequence. | |
| 700380151H1 | g1220121 | 28 | −35 | gb105pln | Tobacco mRNA for S-adenosyl-L-homocysteine hydrolase, complete cds. | |
| 700345836H1 | g949877 | 76 | −59 | gb105pln | H. vulgare mRNA for elongation factor 1-alpha. | |
| 700342273H1 | g2052366 | 7 | 7 | gb105allp | protein 5 | |
| 700345183H1 | g170746 | 20 | −10 | gb105pln | Wheat histone H4 TH091 gene, complete cds. | |
| 700379944H1 | g20255 | 63 | −54 | gb105pln | O. sativa gene for heat shock protein 82 HSP82. | |
| 700344696H1 | g2570129 | 16 | 8 | gb105allp | RVP1 | |
| 700345286H1 | g2668741 | 61 | −55 | gb105pln | Zea mays glycine-rich RNA binding protein (GRP) mRNA, complete cds. | |
| 700346816H1 | g1524234 | 15 | 5 | gb105allp | unknown | |
| 700345841H1 | g159256 | 22 | −4 | gb105eukp | annexin XII; annexin XII | |
| 700347375H1 | g1165205 | 18 | 4 | gb105pln | Arabidopsis thaliana multiubiquitin chain binding protein (MBP1) mRNA, | |
| | | | | | complete cds. | |
| 700347079H1 | g2583106 | 27 | −9 | gb105pln | Arabidopsis thaliana chromosome II BAC F4L23 genomic sequence, complete | |
| | | | | | sequence. | |
| 700346847H1 | g2264320 | 27 | −6 | gb105pln | Arabidopsis thaliana genomic DNA, chromosome 5, P1 clone: MX110, complete | |
| | | | | | sequence. | |
| 700343086H1 | g218334 | 48 | −46 | gb105pln | Triticum aestivum mRNA for O-acetylserine (thiol) lyase. | |
| 700380163H1 | g190234 | 18 | 6 | gb105allp | acidic ribosomal phosphoprotein (P1) | |
| 700379970H1 | g886739 | 65 | −33 | gb105pln | Z. mays histone H4 gene. | |
| 700345108H1 | g18259 | 39 | −37 | gb105pln | c. sativus mRNA for cs DnaJ-1. | |
| 700380044H1 | g2301165 | 10 | 4 | gb105allp | unnamed protein product | |
| 700379868H1 | g168403 | 73 | −1 | gb105pln | Maize actin 1 gene (MAc1), complete cds. | |
| 700344687H1 | g1431628 | 18 | 4 | gb105pln | V. radiata mRNA for pectinacetylesterase. | |
| 700344835H1 | g21802 | 31 | −18 | gb105pln | T. aestivum mRNA for high mobility group protein (HMGW). | |
| 700346136H1 | g1532047 | 36 | −29 | gb105pln | O. sativa mRNA for S-adenosylmethionine decarboxylase. | |
| 700343438H1 | g2408019 | 19 | −6 | gb105eukp | SPAC17G6.06; 40s ribosomal protein | |
| 700347130H1 | g2293567 | 39 | 1 | gb105pln | Oryza sativa HvB12D homolog mRNA, complete cds. | |
| 700342488H1 | g452473 | 43 | −18 | gb105pln | Zea mays Black Mexican Sweet alpha-tubulin mRNA, complete cds. | |
| 700346914H1 | g780371 | 14 | −0 | gb105pln | Oryza sativa enolase mRNA, complete cds. | |
| 700380765H1 | g2827525 | 39 | −9 | gb105eukp | F8F16.120; multicatalytic endopeptidase complex, proteasome precursor, beta subunit | |
| 700342639H1 | g312944 | 17 | −2 | gb105eukp | cysK; cysteine synthase; EC 4.2.99.8 | |
| 700346591H1 | g2239260 | 14 | −7 | gb105eukp | CCR; cinnamoyl CoA reductase | |
| 700379940H1 | g2662344 | 72 | −62 | gb105pln | Oryza sativa mRNA for EF-1 alpha, complete cds. | |
| 700347167H1 | g602605 | 37 | −61 | gb105pln | Zea mays tandem genes for alpha1-tubulin and alpha2-tubulin. | |
| 700347360H1 | g1730332 | 21 | −10 | gb105eukp | (R)-(+)-mandelonitrile lyase isoform MDL1 precursor | |
| 700346785H1 | g218367 | 13 | 10 | gb105pln | Yeast (Candida tropicalis) CTPACTB gene for acetoacetyl-CoA thiolase A. | |
| 700346430H1 | g312178 | 19 | 6 | gb105pln | Z. mays GapC2 gene. | |
| 700380209H1 | g1217976 | 19 | −4 | gb105eukp | SPAC1F12.02c; unknown | |
| 700379731H1 | g166769 | 20 | −4 | gb105pln | A. thaliana heat shock protein 83 mRNA, complete cds. | |
| 700347002H1 | g600768 | 49 | −34 | gb105pln | Oryza sativa cyclophilin 2 (Cyp2) mRNA, complete cds. | |
| 700345755H1 | g1498389 | 54 | −49 | gb105pln | Zea mays actin (Maz81) gene, partial cds. | |
| 700345132H1 | g2274990 | 48 | −5 | gb105pln | Hordeum vulgare mRNA for expressed sequence tag. | |
| 700380212H1 | g168500 | 33 | −42 | gb105pln | Maize (Zea mays) histone H4 gene (H4C14), complete cds. | |
| 700343352H1 | g1279278 | 21 | −3 | gb105eukp | F22D6.2 | |
| 700343377H1 | g2739216 | 24 | 5 | gb105pln | Hordeum vulgare L41 ribosomal protein. | |
| 700343010H1 | g785035 | 9 | −0 | gb105eukp | trypsin; EC 3.4.21.4 | |
| 700342566H1 | g431269 | 16 | 12 | gb105pln | Arabidopsis thaliana ubiquitin conjugating enzyme (UBC5) gene, complete cds. | |
| 700344321H1 | g1574937 | 21 | −31 | gb105pln | Zea mays superoxide dismutase 4 (sod4) gene, partial cds. | |
| 700345694H1 | g1370174 | 35 | −8 | gb105eukp | rab1Y; GTP-binding protein; RAB1Y | |
| 700344916H1 | g218088 | 37 | −29 | gb105pln | Rice mRNA for ribosomal protein 117 (249 gene), partial sequence. | |
| 700342671H1 | g407488 | 12 | 4 | gb105eukp | unknown | |
| 700346033H1 | g620055 | 34 | −17 | gb105eukp | T15H9.6 | |
| 700379674H1 | g2239151 | 24 | 5 | gb105eukp | ch1D; CHLD magnesium chelatase subunit | |
| 700346631H1 | g304108 | 30 | −18 | gb105pln | Arabidopsis thaliana poly(A)-binding protein mRNA, complete cds. | |
| 700379734H1 | g1881536 | 11 | 6 | gb105allp | A20 | |
| 700342206H1 | g2641618 | 53 | −32 | gb105pln | Zea mays ubiquitin-conjugating enzyme protein E2 (ubc7) mRNA, complete cds. | |
| 700342545H1 | g1118094 | 21 | −6 | gb105eukp | C50F7.4 | |
| 700346811H1 | g21600 | 44 | −28 | gb105pln | S. tuberosum mRNA for ubiquitin monomer/ribosomal protein. | |
| 700347264H1 | g1729455 | 34 | −19 | gb105eukp | B0365.1 | |
| 700379891H1 | g510910 | 35 | −25 | gb105pln | L. temulentum mRNA for histone H3. | |
| 700344425H1 | g1381150 | 26 | 0 | gb105allp | Hs-CUL-4B | |
| 700344661H1 | g218082 | 53 | −15 | gb105pln | Rice mRNA for initiation factor eIF-4D (225 gene), partial sequence. | |
| 700353606H1 | g1255861 | 23 | −5 | gb105eukp | F02E8.3 | |
| 700355772H1 | g2190016 | 6 | 8 | gb105eukp | alliinase; EC 4.4.1.4 | |
| 700353969H1 | g2154727 | 26 | −2 | gb105pln | C. dactylon mRNA for profilin 2. | |
| 700353206H1 | g669003 | 23 | −5 | gb105eukp | calnexin | |
| 700282404H2 | g965081 | 4 | 8 | gb105eukp | YPL012W; Yp1012wp | |
| 700356833H1 | g311686 | 19 | −11 | gb105eukp | 3-ketoacyl-acyl carrier protein synthase III (KAS III) | |
| 700353708H1 | g22418 | 21 | −16 | gb105pln | Z. mays gene for polygalacturonase (clone 17). | |
| 700355859H1 | g474390 | 64 | −58 | gb105pln | H. vulgare L. (Alexis) Serine carobxypeptidase II-2 mRNA. | |
| 700352886H1 | g17789 | 16 | −0 | gb105eukp | Bp10 | |
| 700383143H1 | g64876 | 6 | 7 | gb105allp | La protein form B | |
| 700355883H1 | g22456 | 97 | −91 | gb105pln | Z. mays pollen specific mRNA C-terminal (clone 3C12). | |
| 700356115H1 | g288366 | 100 | −28 | gb105pln | Z. mays PGg14 gene for polygalacturonase. | |
| 700352725H1 | g1772496 | 20 | −27 | gb105eukp | peptidyl-prolyl cis-trans isomerase.; EC 5.2.1.8 | |
| 700354794H1 | g1438883 | 16 | 1 | gb105allp | GmCK3p | |
| 700354442H1 | g1293783 | 53 | −32 | gb105pln | Oryza sativa QM gene, complete cds. | |
| 700353688H1 | g20310 | 15 | 0 | gb105eukp | pollen specific gene | |
| 700382825H1 | g1302033 | 24 | −9 | gb105pln | Yeast (Saccharomyces cerevisiae) chromosome XIV reading frame ORF YNL112w. | |
| 700353164H1 | g22118 | 25 | 2 | gb105pln | Z. mays DNA for Adh1-Cm allele. | |
| 700352644H1 | g499011 | 66 | −71 | gb105pln | S. vulgare SoAc1 mRNA. | |
| 700383102H1 | g288378 | 45 | −34 | gb105pln | Z. mays gene PG for polygalacturonase. | |
| 700354367H1 | g1519252 | 23 | −49 | gb105pln | Oryza sativa GF14-d protein mRNA, complete cds. | |
| 700352905H1 | g2316063 | 28 | −9 | gb105eukp | cyt-b; cytochrome apoenzyme b | |
| 700353022H1 | g414702 | 34 | −10 | gb105pln | H. lanatus mRNA for allergen Hol-1I. | |
| 700353405H1 | g998429 | 55 | −40 | gb105pln | GRF1 = general regulatory factor [Zea mays, XL80, Genomic, 5348 nt]. | |
| 700353950H1 | g288373 | 42 | −44 | gb105pln | Z. mays PGg6 gene for polygalacturonase. | |
| 700352994H1 | g296094 | 78 | −3 | gb105eukp | M(3)95A; ribosomal protein S3 | |
| 700282438H2 | g2695929 | 15 | 4 | gb105allp | putative thiol protease | |
| 700356670H1 | g1743414 | 32 | −3 | gb105eukp | pSB5; transmembrane proton pump | |
| 700282471H2 | g2181190 | 10 | 8 | gb105allp | serine/threonine kinase | |
| 700355985H1 | g2266661 | 42 | −24 | gb105pln | Hordeum vulgare mRNA for 14-3-3 protein (Hv1433c). | |
| 700355446H1 | g17795 | 26 | 2 | gb105allp | Bplo gene product | |
| 700355973H1 | g544501 | 11 | −3 | gb105eukp | SEC13; Sec13p | |
| 700382952H1 | g313137 | 11 | −1 | gb105pln | Z. mays ZmPRO1 mRNA for profilin 1. | |
| 700352804H1 | g1030068 | 18 | −4 | gb105eukp | NAD(P)H oxidoreductase, isoflavone reductase homologue | |
| 700382314H1 | g1184773 | 96 | −82 | gb105pln | Zea mays glyceraldehyde-3-phosphate dehydrogenase GAPC3 (gpc3) mRNA, | |
| | | | | | complete cds. | |
| 700382087H1 | g1688322 | 7 | 3 | gb105eukp | pinA; pinA | |
| 700356985H1 | g457688 | 56 | −14 | gb105pln | M. crystallinum mRNA for protein kinase. | |
| 700282545H2 | g255568 | 15 | 16 | gb105pln | pollen specific protein [Zea mays = corn, mRNA, 943 nt]. | |
| 700382807H1 | g22560 | 27 | −18 | gb105pln | Mesembryanthemum crystallinum ppc2 gene for phosphoenolpyruvate carboxylase | |
| | | | | | (EC 4.1.1.31). | |
| 700355911H1 | g1771781 | 24 | −22 | gb105pln | T. aestivum mRNA for beta-amylase. | |
| 700355324H1 | g517065 | 25 | 6 | gb105allp | chaperonin-like protein | |
| 700353987H1 | g22427 | 53 | 6 | gb105pln | Z. mays gene for polygalacturonase (clone 65). | |
| 700354407H1 | g2244792 | 11 | −3 | gb105eukp | ankyrin homolog | |
| 700354922H1 | g1785739 | 6 | 7 | gb105eukp | tRNA-Ser | |
| 700356606H1 | g809535 | 21 | 15 | gb105pln | B. verrucosa mRNA for BETV4 pollen allergen. | |
| 700355754H1 | g473359 | 20 | −28 | gb105pln | P. pratense mRNA for Phl pI allergen. | |
| 700382304H1 | g902583 | 39 | −79 | gb105pln | Zea mays clone MubG1 ubiquitin gene, complete cds. | |
| 700356848H1 | g2160155 | 10 | 9 | gb105pln | Sequence of BAC F21M12 from Arabidopsis thaliana chromosome 1, complete | |
| | | | | | sequence. | |
| 700354582H1 | g22458 | 62 | −59 | gb105pln | Z. mays pollen specific mRNA C-terminal (clone 4H7). | |
| 700382574H1 | g288611 | 28 | −15 | gb105pln | Z. mays PG gene for polygalacturonase. | |
| 700383159H1 | g22428 | 8 | 5 | gb105eukp | polygalacturonase | |
| 700382094H1 | g1777706 | 46 | −75 | gb105pln | Zea mays 18S ribosomal RNA gene, partial sequence. | |
| 700357106H1 | g497900 | 86 | 5 | gb105eukp | S-adenosyl methionine synthetase | |
| 700354644H1 | g1184773 | 100 | −80 | gb105pln | Zea mays glyceraldehyde-3-phosphate dehydrogenase GAPC3 (gpc3) mRNA, | |
| | | | | | complete cds. | |
| 700354657H1 | g218175 | 51 | −47 | gb105pln | Rice mRNA for heat shock protein 82 (NH67 gene), partial sequence. | |
| 700282576H2 | g1658173 | 39 | −23 | gb105eukp | otaA; ornithine transaminase; EC 2.6.1.13 | |
| 700355858H1 | g2511530 | 36 | −39 | gb105pln | Eleusine indica alpha tubulin 1 (TUA1) mRNA, complete cds. | |
| 700383156H1 | g414702 | 22 | −3 | gb105pln | H. lanatus mRNA for allergen Hol-lI. | |
| 700357145H1 | g416265 | 42 | −32 | gb105pln | Rice mRNA for ribosomal protein A2, partial sequence. | |
| 700353291H1 | g1777414 | 19 | 3 | gb105allp | 26S proteasome subunit | |
| 700382150H1 | g2252869 | 25 | −10 | gb105eukp | AT0ZI1 | |
| 700354493H1 | g1438878 | 26 | −7 | gb105pln | Glycine max choline kinase GmCK1p mRNA, complete cds. | |
| 700353442H1 | g487946 | 23 | −23 | gb105eukp | DYS1; Dys1p: Deoxyhypusine synthase | |
| 700353084H1 | g2114105 | 32 | −12 | gb105pln | Arabidopsis thaliana mRNA for sulfate transporter, complete cds. | |
| 700357406H1 | g971853 | 67 | 3 | gb105allp | expressed sequence tag; human homolog | |
| 700352655H1 | g777757 | 49 | −59 | gb105pln | Saccharum hybrid (clone SCUBI561) polyubiquitin mRNA, complete cds. | |
| 700383157H1 | g974781 | 38 | −26 | gb105pln | C. blumei kinetoplast met gene for cobalamine-independent methionine synthase. | |
| 700353959H1 | g2062421 | 12 | 1 | gb105eukp | LeEx1; expansin | |
| 700357419H1 | g531389 | 18 | −6 | gb105eukp | gag/pol; copia-like retrotransposon Hopscotch polyprotein | |
| 700382308H1 | g168460 | 55 | −75 | gb105pln | Zea mays cyclophilin (CyP) mRNA, complete cds. | |
| 700355436H1 | g22237 | 62 | −68 | gb105pln | Maize mRNA for cytosolic GAPDH (GapC) glyceraldehyde-3-phosphate | |
| | | | | | dehydrogenase. | |
| 700355582H1 | g1549222 | 18 | −8 | gb105eukp | NtSAR1; NtSar1 protein | |
| 700356263H1 | g607895 | 9 | 1 | gb105eukp | RTS2; anther specific | |
| 700382877H1 | g414702 | 27 | −2 | gb105pln | H. lanatus mRNA for allergen Hol-lI. | |
| 700383030H1 | g176779 | 32 | −16 | gb105allp | gamma-2 globin | |
| 700382112H1 | g1885343 | 32 | −57 | gb105pln | T. estivum mRNA for starch branching enzyme II. | |
| 700354788H1 | g2344871 | 25 | −16 | gb105eukp | pap; purple acid phosphatase precursor; EC 3.1.3.2 | |
| 700353220H1 | g2464894 | 30 | −18 | gb105pln | Arabidopsis thaliana DNA chromosome 4, ESSA I AP2 contig fragment No. 2. | |
| 700355767H1 | g2190559 | 10 | 1 | gb105eukp | F5I14.16; F5I14.16 | |
| 700382015H1 | g16513 | 25 | −40 | gb105pln | A. thaliana gene for suppressor-like protein. | |
| 700382044H1 | g22425 | 92 | −85 | gb105pln | Z. mays gene for polygalacturonase (clone 47). | |
| 700354322H1 | g22418 | 44 | −38 | gb105pln | Z. mays gene for polygalacturonase (clone 17). | |
| 700382730H1 | g1374698 | 27 | −7 | gb105allp | nuclear protein, NP220 | |
| 700356209H1 | g1184773 | 34 | 1 | gb105pln | Zea mays glyceraldehyde-3-phosphate dehydrogenase GAPC3 (gpc3) mRNA, | |
| | | | | | complete cds. | |
| 700356086H1 | g595816 | 9 | 3 | gb105eukp | mudrA | |
| 700352826H1 | g287830 | 14 | −3 | gb105eukp | polygalacturonase | |
| 700356282H1 | g296385 | 18 | 9 | gb105pln | N. tabacum mRNA for cytochrome b5. | |
| 700383167H1 | g435172 | 27 | −3 | gb105pln | A. sativa (Pewi) ASTCP-K19 mRNA for t complex polypeptide 1. | |
| 700382436H1 | g550019 | 6 | 5 | gb105allp | ribosomal protein L28 | |
| 700356834H1 | g288366 | 82 | −80 | gb105pln | Z. mays PGg14 gene for polygalacturonase. | |
| 700356095H1 | g1790315 | 9 | 8 | gb105allp | hypothetical 31.2 kD protein in glnA-fdhE intergenic region | |
| 700382279H1 | g2239262 | 8 | 7 | gb105allp | pectin methylesterase-like protein | |
| 700354794H1 | g1438879 | 15 | 1 | gb105eukp | GmCK1p; EC 2.7.1.32 | |
| 700382885H1 | g643596 | 67 | −58 | gb105pln | Corn mRNA for cysteine proteinase, clone CCP, complete cds. | |
| 700352677H1 | g429015 | 52 | −39 | gb105pln | Rice mRNA for ribosomal protein (gene name SS498), partial cds. | |
| 700382721H1 | g20321 | 53 | −45 | gb105pln | Oryza sativa RAc1 mRNA for actin. | |
| 700382331H1 | g736338 | 34 | −32 | gb105pln | Glycine max phosphatidylinositol 3-kinase mRNA, complete cds. | |
| 700353120H1 | g2642323 | 43 | −38 | gb105pln | Zea mays profilin (PRO4) mRNA, complete cds. | |
| 700355264H1 | g21800 | 86 | −4 | gb105pln | T. aestivum L mRNA for histone H2B. | |
| 700352848H1 | g22425 | 34 | −22 | gb105pln | Z. mays gene for polygalacturonase (clone 47). | |
| 700282516H2 | g18317 | 14 | −3 | gb105pln | D. carota RNA for actin gene Ac1. | |
| 700382579H1 | g2245024 | 13 | 1 | gb105eukp | cyanohydrin lyase homolog | |
| 700355062H1 | g2828278 | 15 | −5 | gb105pln | Arabidopsis thaliana DNA chromosome 4, BAC clone T18B16 (ESSAII project). | |
| 700352630H1 | g927544 | 14 | 15 | gb105pln | Yeast (Saccharomyces cerevisiae) YPT6 gene. | |
| 700355941H1 | g747914 | 90 | −76 | gb105pln | Z. mays CaM1 mRNA for calmodulin. | |
| 700353129H1 | g1399549 | 12 | −0 | gb105pln | Anemone trifolia nuclear 26S ribosomal RNA gene, partial sequence. | |
| 700354442H1 | g468055 | 69 | −45 | gb105pln | Zea mays B73 QM protein mRNA, complete cds. | |
| 700382947H1 | g288378 | 97 | −44 | gb105pln | Z. mays gene PG for polygalacturonase. | |
| 700355086H1 | g1244652 | 51 | 8 | gb105pln | Zea mays copia-type retroelement PREM-2 gag gene, complete cds. | |
| 700355972H1 | g337455 | 37 | −24 | gb105allp | hnRNP C2 protein | |
| 700355868H1 | g2735007 | 66 | −79 | gb105pln | Zea mays kinase associated protein phosphatase (KAPP) mRNA, complete cds. | |
| 700355083H1 | g887785 | 27 | −13 | gb105eukp | ILR1; ILR1 hydrolyzes certain amino acid conjugates of the plant growth regulator | |
| | | | | | indole-3-acetic acid (IAA), including IAA-Leu and IAA-Phe; ILR1 | |
| 700354787H1 | g2464935 | 23 | −6 | gb105eukp | serine C-palmitoyltransferase homolog | |
| 700354929H1 | g414702 | 22 | −5 | gb105pln | H. lanatus mRNA for allergen Hol-lI. | |
| 700354737H1 | g2291232 | 8 | 4 | gb105allp | similar to the AAA family of ATPases | |
| 700357452H1 | g16509 | 41 | −30 | gb105pln | A. thaliana mRNA for succinate-CoA ligase (GCP-forming). | |
| 700282439H2 | g596079 | 37 | −56 | gb105pln | Zea mays thiamine biosynthetic enzyme (thi1-2) mRNA, complete cds. | |
| 700382407H1 | g2182287 | 24 | −6 | gb105pln | Genomic sequence for Arabidopsis thaliana BAC T7N9, complete sequence. | |
| 700354681H1 | g453975 | 41 | −22 | gb105pln | P. pratense mRNA for profilin-Phl p XI. | |
| 700352617H1 | g313139 | 72 | −79 | gb105pln | Z. mays ZmPRO2 mRNA for profilin 2. | |
| 700382157H1 | g1184773 | 83 | −70 | gb105pln | Zea mays glyceraldehyde-3-phosphate dehydrogenase GAPC3 (gpc3) mRNA, | |
| | | | | | complete cds. | |
| 700355762H1 | g19902 | 25 | −10 | gb105eukp | pollen specific protein | |
| 700354644H1 | g312180 | 39 | −64 | gb105pln | Z. mays GapC4 gene. | |
| 700353429H1 | g20359 | 72 | −5 | gb105pln | Rice gene for 17S ribosomal RNA. | |
| 700382474H1 | g433044 | 16 | 12 | gb105pln | Zea mays W-22 clone PREM-1D retroelement PREM-1, partial sequence. | |
| 700352852H1 | g288378 | 93 | −77 | gb105pln | Z. mays gene PG for polygalacturonase. | |
| 700382142H1 | g1132482 | 60 | −40 | gb105pln | Rice mRNA for ADP-ribosylation factor, complete cds. | |
| 700355313H1 | g1638836 | 54 | −50 | gb105pln | H. vulgare mRNA for alpha-tubulin 2. | |
| 700355208H1 | g2505871 | 87 | −6 | gb105eukp | ribosomal protein S18A | |
| 700354319H1 | g1707641 | 12 | 13 | gb105pln | O. sativa mRNA for leucine rich repeat receptor-like kinase. | |
| 700353549H1 | g600768 | 44 | 10 | gb105pln | Oryza sativa cyclophilin 2 (Cyp2) mRNA, complete cds. | |
| 700354923H1 | g1403043 | 37 | −30 | gb105pln | H. chilense x T. turgidum conv. durum (Tritordeum) mRNA for S-adenosylmethionine | |
| | | | | | decarboxylase. | |
| 700353565H1 | g2435604 | 20 | 7 | gb105eukp | F08F1.7 | |
| 700382615H1 | g2801802 | 24 | −6 | gb105pln | Oryza sativa germin-like protein 16 (glp16) mRNA, complete cds. | |
| 700382147H1 | g1497983 | 15 | 0 | gb105eukp | alien; putative thyroid receptor interacting protein | |
| 700357439H1 | g2565420 | 33 | 5 | gb105pln | Onobrychis viciifolia eukaryotic translation initiation factor eIF-1A mRNA, | |
| | | | | | complete cds. | |
| 700356170H1 | g414702 | 21 | −0 | gb105pln | H. lanatus mRNA for allergen Hol-lI. | |
| 700382773H1 | g2351070 | 13 | 11 | gb105pln | Arabidopsis thaliana genomic DNA, chromosome 5, P1 clone: MTH12. | |
| 700355311H1 | g1015931 | 24 | −4 | gb105eukp | ribosomal protein L1 | |
| 700354577H1 | g1155333 | 46 | −4 | gb105pln | Solanum tuberosum POTATP1 mRNA, complete cds. | |
| 700355771H1 | g167607 | 16 | −1 | gb105eukp | actin 7 | |
| 700352629H1 | g915313 | 23 | 3 | gb105allp | ribosomal protein L31 | |
| 700356574H1 | g287829 | 68 | −75 | gb105pln | Z. mays gene for polygalacturonase. | |
| 700355780H1 | g387431 | 18 | 7 | gb105allp | metallothionien II | |
| 700282445H2 | g1741870 | 26 | −11 | gb105allp | SDF2 | |
| 700353109H1 | g287834 | 73 | −8 | gb105pln | Z. mays yptm2 cDNA. | |
| 700356136H1 | g540532 | 25 | −39 | gb105pln | Rice mRNA for G protein alpha subunit, complete cds. | |
| 700353716H1 | g1899059 | 54 | −61 | gb105pln | Zea mays endosperm C-24 sterol methyltransferase (ESMT1) mRNA, complete cds. | |
| 700282505H2 | g532571 | 23 | −7 | gb105pln | Barley lipoxygenase 1 (LoxA) gene, complete cds. | |
| 700357429H1 | g393400 | 77 | −46 | gb105pln | Z. mays mRNA for alpha-tubulin. | |
| 700355046H1 | g485376 | 84 | −76 | gb105pln | Zea mays alpha-3-tubulin gene, complete cds. | |
| 700354627H1 | g1707856 | 40 | −46 | gb105pln | T. aestivum mRNA for obtusifoliol 14-alpha-demethylase (CYP51), clone w516, | |
| | | | | | partial. | |
| 700382205H1 | g2244950 | 24 | −23 | gb105pln | Arabidopsis thaliana DNA chromosome 4, ESSA I contig fragment No. 5. | |
| 700356294H1 | g1654348 | 7 | 8 | gb105allp | cytoplasmatic phosphoprotein | |
| 700355823H1 | g2429617 | 48 | −59 | gb105pln | Oryza sativa mRNA for ferrochelatase, partial cds. | |
| 700357140H1 | g1223922 | 22 | −7 | gb105eukp | vicilin peptidohydrolase | |
| 700352939H1 | g1902903 | 12 | 5 | gb105allp | phospholipase D | |
| 700382574H1 | g288378 | 27 | −13 | gb105pln | Z. mays gene PG for polygalacturonase. | |
| 700353952H1 | g1049082 | 14 | 5 | gb105allp | SRp40-3 | |
| 700354368H1 | g17862 | 30 | −22 | gb105pln | B. napus mRNA for ribosomal protein S15a (pPCB8). | |
| 700355419H1 | g1289203 | 32 | −17 | gb105pln | A. glutinosa mRNA for thiazole biosynthetic enzyme. | |
| 700353902H1 | g1236961 | 25 | −8 | gb105eukp | hydrolysis of the cyanogenic glucoside (R)-prunasin; prunasin hydrolase precursor; | |
| | | | | | EC 3.2.1.21 | |
| 700353263H1 | g999395 | 53 | −52 | gb105pln | HSP81-3 = heat-shock Protein [Arabidopsis thaliana = thale-cress, Genomic, 3094 nt]. | |
| 700355243H1 | g393449 | 82 | −18 | gb105pln | S. cereale mRNA for beta-amylase. | |
| 700382822H1 | g288611 | 48 | −48 | gb105pln | Z. mays PG gene for polygalacturonase. | |
| 700355264H1 | g473604 | 82 | −2 | gb105pln | Zea mays W-22 histone H2B mRNA, complete cds. | |
| 700355262H1 | g255568 | 75 | −59 | gb105pln | pollen specific protein [Zea mays = corn, mRNA, 943 nt]. | |
| 700383131H1 | g475257 | 18 | 7 | gb105eukp | similar to ribosomal protein L19; ctg start codon. putative | |
| 700356461H1 | g499655 | 14 | 8 | gb105eukp | gamma-thionin homolog | |
| 700282445H2 | g1431130 | 5 | 8 | gb105eukp | PMT1 | |
| 700353276H1 | g2656025 | 37 | −28 | gb105pln | Arabidopsis thaliana genomic DNA, chromosome 5, P1 clone: MCD7. | |
| 700353442H1 | g1019423 | 24 | −22 | gb105eukp | catalyzes the transfer of the aminobutyl group from spermidine to eIF-5A lysine | |
| | | | | | residue; deoxyhypusine synthase | |
| 700352874H1 | g1086780 | 13 | 5 | gb105eukp | T02G5.4 | |
| 700356113H1 | g168316 | 17 | −3 | gb105eukp | Lol pI; pollen allergen | |
| 700382489H1 | g186192 | 50 | −41 | gb105allp | immunoglobulin mu chain | |
| 700353106H1 | g1841869 | 18 | 9 | gb105pln | Pimpinella brachycarpa elongation factor 1-beta (EF-1-beta) mRNA, complete cds. | |
| 700356463H1 | g666087 | 68 | 1 | gb105allp | carbonyl reductase (NADPH) | |
| 700382607H1 | g533692 | 22 | 1 | gb105eukp | protease inhibitor | |
| 700354602H1 | g763158 | 22 | −6 | gb105allp | GTP-binding protein | |
| 700353716H1 | g1706964 | 40 | −27 | gb105pln | Triticum aestivum delta-24-sterol methyltransferase (TA-MT) mRNA, complete cds. | |
| 700382546H1 | g398846 | 48 | −26 | gb105pln | Z. mays mRNA for beta 4 tubulin. | |
| 700355264H1 | g1066160 | 80 | −2 | gb105pln | Wheat gene for protein H2B153, complete cds. | |
| 700282591H2 | g409499 | 23 | 5 | gb105allp | 3-Hydroxy-3-methylglutaryl coenzyme A lyase, HL {EC 4.1.3.4} [mice, liver, Peptide | |
| | | | | | Mitochondrial, 325 aa] | |
| 700355330H1 | g16110 | 47 | −48 | gb105pln | Avena sativa mRNA for phytochrome (AP3). | |
| 700356819H1 | g1679757 | 10 | 3 | gb105eukp | chk-1; two-component histidine kinase CHK-1 | |
| 700355771H1 | g20637 | 19 | −3 | gb105eukp | actin | |
| 700352920H1 | g35555 | 28 | 7 | gb105allp | PM/Scl 100kD nucleolar protein | |
| 700357147H1 | g2570506 | 44 | −48 | gb105pln | Oryza sativa ribosomal protein mRNA, complete cds. | |
| 700354360H1 | g1616660 | 94 | −72 | gb105pln | Zea mays adenylosuccinate synthetase mRNA, complete cds. | |
| 700354705H1 | g2244842 | 10 | 1 | gb105eukp | hypothetical protein | |
| 700355955H1 | g1632809 | 18 | 5 | gb105allp | NADH dehydrogenase subunit 3 | |
| 700352923H1 | g22416 | 100 | −57 | gb105pln | Z. mays mRNA for polygalacturonase (clone PG1). | |
| 700355780H1 | g31435 | 21 | 6 | gb105allp | metallothionein | |
| 700353429H1 | g1777706 | 72 | −5 | gb105pln | Zea mays 18S ribosomal RNA gene, partial sequence. | |
| 700355095H1 | g172423 | 24 | 3 | gb105pln | Yeast (Saccharomyces cerevisiae) robosomal protein L1 gene, complete cds. | |
| 700354738H1 | g575730 | 41 | −63 | gb105pln | Z. mays mRNA for transmembrane protein. | |
| 700356573H1 | g974781 | 43 | −41 | gb105pln | C. blumei kinetoplast met gene for cobalamine-independent methionine sythase. | |
| 700355060H1 | g472849 | 14 | 17 | gb105pln | Glycine max Essex desiccation protectant protein Lea14 homolog mRNA, | |
| | | | | | complete cds. | |
| 700356127H1 | g520935 | 34 | −26 | gb105pln | H. vulgare mRNA for gamma-TIP-like protein. | |
| 700354415H1 | g1480025 | 21 | −2 | gb105pln | Brassica rapa mRNA, polyA_site. | |
| 700352887H1 | g1747295 | 66 | −16 | gb105pln | Rice mRNA for vacuolar H+-pyrophosphatase, complete cds. | |
| 700383175H1 | g414702 | 27 | −11 | gb105pln | H. lanatus mRNA for allergen Hol-lI. | |
| 700382383H1 | g1145694 | 34 | −21 | gb105pln | Arabidopsis thaliana actin (ACT3) | |
| 700353267H1 | g2351062 | 16 | 10 | gb105pln | Arabidopsis thaliana genomic DNA chromosome 5, P1 clone: MAH20. | |
| 700355878H1 | g293901 | 16 | −6 | gb105pln | Zea mays mI gene, complete cds. | |
| 700355829H1 | g168558 | 44 | 4 | gb105eukp | putative. similar to GTP-binding proteins | |
| 700355560H1 | g1911581 | 30 | −15 | gb105pln | Cyn d 1 = major allergen {clone 14c1 and CD1} [Cynodon dactylon = Bermuda | |
| | | | | | grass, pollen, mRNA Partial, 759 nt]. | |
| 700353022H1 | g414907 | 33 | −10 | gb105pln | P. pratense mRNA for pollen allergen PhlpI. | |
| 700356745H1 | g18917 | 20 | 6 | gb105pln | Barley mRNA for beta-amylase. | |
| 700356507H1 | g1185555 | 29 | −18 | gb105pln | Zea mays glyceraldehyde-3-phosphate dehydrogenase (gpc4) gene, partial cds. | |
| 700353981H1 | g485376 | 79 | −72 | gb105pln | Zea mays alpha-3-tubulin gene, complete cds. | |
| 700353171H1 | g393449 | 42 | −0 | gb105pln | S. cereale mRNA for beta-amylase. | |
| 700352952H1 | g22416 | 100 | −87 | gb105pln | Z. mays mRNA for polygalacturonase (clone PG1). | |
| 700382032H1 | g1813887 | 16 | 7 | gb105allp | hypothetical protein (cDNA194) | |
| 700353234H1 | g20163 | 19 | −24 | gb105pln | O. sativa Rrl5 mRNA for 5S ribosomal RNA. | |
| 700282554H2 | g2463582 | 10 | 4 | gb105eukp | precursor of cysteine proteinase; FB31 precursor (FB13 precursor) | |
| 700383022H1 | g414907 | 26 | −17 | gb105pln | P. pratense mRNA for pollen allergen PhlpI. | |
| 700353945H1 | g493588 | 31 | −11 | gb105pln | Hordeum vulgare disulfide isomerase (PDI) mRNA, complete cds. | |
| 700356033H1 | g2282586 | 11 | 3 | gb105eukp | methyltransferase | |
| 700353291H1 | g1155334 | 24 | 2 | gb105eukp | POTATP1 | |
| 700356901H1 | g1402878 | 17 | 6 | gb105eukp | orf4; unknown | |
| 700356273H1 | g12956 | 54 | 4 | gb105allp | reading frame protein 5 (896 is 2nd base in codon) | |
| 700355696H1 | g287830 | 11 | 8 | gb105eukp | polygalacturonase | |
| 700382111H1 | g436051 | 56 | −62 | gb105pln | Rice mRNA for branching enzyme-3, complete cds. | |
| 700353091H1 | g167109 | 34 | −45 | gb105pln | Hordeum vulgare vacuolar ATPase B subunit mRNA, complete cds. | |
| 700353867H1 | g17795 | 16 | −3 | gb105eukp | Bplo | |
| 700354373H1 | g620041 | 34 | −26 | gb105pln | A. adeninivorans TEF gene for translation elongation factor EF-1alpha. | |
| 700353951H1 | g1621306 | 10 | 6 | gb105allp | F21G4.2 | |
| 700353027H1 | g2266661 | 50 | −17 | gb105pln | Hordeum vulgare mRNA for 14-3-3 protein (Hv1433c). | |
| 700357106H1 | g609559 | 86 | 5 | gb105eukp | S-adenosylmethionine synthase; EC 2.5.1.6 | |
| 700282464H1 | g22426 | 13 | 1 | gb105eukp | polygalacturonase | |
| 700357106H1 | g1724104 | 86 | 5 | gb105eukp | S-adenosylmethionine sythetase; methionine adenosyltransferase; EC 2.5.1.6 | |
| 700383156H1 | g414907 | 24 | −5 | gb105pln | P. pratense mRNA for pollen allergen PhlpI. | |
| 700382338H1 | g1246115 | 32 | −19 | gb105pln | major allergen Pha a 1 [Phalaris aquatica = canary grass, pollen, mRNA Partial, | |
| | | | | | 1088 nt]. | |
| 700356205H1 | g1785947 | 22 | −7 | gb105pln | Hordeum vulgare cytosolic triosephosphate isomerase mRNA, complete cds. | |
| 700354457H1 | g288373 | 37 | −55 | gb105pln | Z. mays PGp6 gene for polygalacturonase. | |
| 700382741H1 | g204667 | 26 | −1 | gb105allp | 70 kDa heat-shock-like protein | |
| 700382091H1 | g22428 | 17 | −10 | gb105eukp | polygalacturonase | |
| 700354425H1 | g1657854 | 12 | 12 | gb105pln | Triticum aestivum cold acclimation protein WCOR413 (Wcor413) mRNA, | |
| | | | | | complete cds. | |
| 700382036H1 | g2842490 | 8 | 6 | gb105eukp | F21O9.160; heat-shock protein | |
| 700354696H1 | g2154729 | 51 | −36 | gb105pln | C. dactylon mRNA for profilin 1. | |
| 700382820H1 | g206730 | 14 | 3 | gb105allp | ribosomal protein L35 | |
| 700356917H1 | g2264318 | 49 | −11 | gb105pln | Arabidopsis thaliana genomic DNA, chromosome 5, P1 clone: MUP24, complete | |
| | | | | | sequence. | |
| 700382849H1 | g1498391 | 44 | −68 | gb105pln | Zea mays actin (Maz65) gene, partial cds. | |
| 700354918H1 | g22428 | 12 | 7 | gb105eukp | polygalacturonase | |
| 700353688H1 | g255569 | 13 | 2 | gb105eukp | pollen specific protein | |
| 700355774H1 | g473205 | 13 | 17 | gb105pln | E. gunnii mRNA for mitochondrial malate dehydrogenase. | |
| 700355711H1 | g1617036 | 36 | −11 | gb105eukp | Ted2 | |
| 700356916H1 | g15988 | 76 | −25 | gb105pln | A. fatua mRNA for tetraubiquitin. | |
| 700353405H1 | g168602 | 65 | −30 | gb105pln | Zea mays regulatory protein GF14-12 mRNA, complete cds. | |
| 700382877H1 | g414907 | 26 | −2 | gb105pln | P. pratense mRNA for pollen allergen PhlpI. | |
| 700382749H1 | g1167836 | 27 | 3 | gb105allp | protein with incomplete signal sequence | |
| 700354714H1 | g1657619 | 13 | −3 | gb105eukp | AtG5; putative transmembrane protein; G5p | |
| 700354360H1 | g1616656 | 30 | −14 | gb105pln | Arabidopsis thaliana adenylosuccinate synthetase mRNA, complete cds. | |
| 700353524H1 | g20835 | 42 | 7 | gb105eukp | PHSP1; HSP70 | |
| 700356901H1 | g2252840 | 17 | 6 | gb105allp | contains regions of similarity to Haemophilus influenzae permease (SP:P38767) | |
| 700355035H1 | g975887 | 29 | −10 | gb105pln | Mesembryanthemum crystallinum myo-inositol-1-phosphate synthase mRNA, | |
| | | | | | complete cds. | |
| 700355593H1 | g414702 | 19 | −8 | gb105pln | H. lanatus mRNA for allergen Hol-lI. | |
| 700354362H1 | g288373 | 38 | −58 | gb105pln | Z. mays PGg6 gene for polygalacturonase. | |
| 700352642H1 | g1103627 | 36 | −76 | gb105pln | Z. mays Fer1 gene. | |
| 700354801H1 | g2454183 | 22 | −18 | gb105pln | Arabidopsis thaliana pyruvate dehydrogenase E1 beta subunit mRNA, nuclear gene | |
| | | | | | encoding plastid protein, complete cds. | |
| 700356069H1 | g2058277 | 59 | 15 | gb105pln | A. thaliana mRNA for AtRan1 protein. | |
| 700355918H1 | g1695882 | 11 | 1 | gb105allp | mitotic centromere-associated kinesin | |
| 700355741H1 | g166769 | 13 | 11 | gb105pln | A. thaliana heat shock protein 83 mRNA, complete cds. | |
| 700382004H1 | g561663 | 25 | −56 | gb105pln | Rice mRNA, partial homologous to ribosomal protein coding sequence. | |
| 700356533H1 | g2290202 | 7 | 6 | gb105allp | nucellin | |
| 700382822H1 | g288378 | 37 | −32 | gb105pln | Z. mays gene PG for polygalacturonase. | |
| 700354938H1 | g2154729 | 34 | −20 | gb105pln | C. dactylon mRNA for profilin 1. | |
| 700352647H1 | g1065515 | 11 | 5 | gb105eukp | F40F4.7 | |
| 700355555H1 | g2662342 | 46 | −63 | gb105pln | Oryza sativa mRNA for EF-1 alpha, complete cds. | |
| 700354894H1 | g287829 | 60 | −67 | gb105pln | Z. mays gene for polygalacturonase. | |
| 700353994H1 | g441216 | 54 | −16 | gb105pln | Tobacco mRNA for cytokinin binding protein CBP57 (S-adenosyl-L-homocystein | |
| | | | | | hydrolase), complete cds. | |
| 700353685H1 | g886403 | 25 | 4 | gb105allp | MADS box protein | |
| 700382787H1 | g348695 | 18 | −12 | gb105pln | Catharanthus roseus heat shock protein 90 mRNA, complete cds. | |
| 700357412H1 | g308902 | 26 | −8 | gb105eukp | pectate lyase | |
| 700354374H1 | g1480465 | 16 | −6 | gb105eukp | cyclophilin Ovcyp-2 | |
| 700356810H1 | g1931636 | 26 | −8 | gb105pln | Arabidopsis thaliana BAC T19D16 genomic sequence. | |
| 700354259H1 | g974781 | 34 | −41 | gb105pln | C. blumei kinetoplast met gene for cobalamine-independent methionine synthase. | |
| 700382133H1 | g2245394 | 50 | −6 | gb105eukp | putative transcription factor; ARF1-binding protein | |
| 700356814H1 | g2224914 | 27 | −25 | gb105pln | Oryza sativa beta-expansin mRNA, complete cds. | |
| 700356256H1 | g248338 | 73 | −85 | gb105pln | polyubiquitin [maize, Genomic, 3439 nt]. | |
| 700354604H1 | g1330350 | 10 | 2 | gb105eukp | C34D4.2 | |
| 700382510H1 | g22421 | 63 | −61 | gb105pln | Z. mays mRNA for polygalacturonase (clone PG3). | |
| 700382152H1 | g755711 | 14 | 6 | gb105eukp | R05H5.3 | |
| 700356413H1 | g22483 | 52 | −44 | gb105pln | Z. mays RNA for superoxide dismutase Sod4. | |
| 700354544H1 | g1946300 | 32 | 1 | gb105eukp | hypothetical protein | |
| 700353816H1 | g2642213 | 26 | 1 | gb105eukp | nitrate-induced NOI protein | |
| 700353606H1 | g2231702 | 15 | 1 | gb105eukp | AAP19-2; clathrin assembly protein AP19 homolog | |
| 700353569H1 | g2149050 | 31 | −12 | gb105pln | Arabidopsis thaliana small Ras-like GTP-binding protein (Ran3) mRNA, | |
| | | | | | complete cds. | |
| 700352620H1 | g20359 | 95 | −82 | gb105pln | Rice gene for 17S ribosomal RNA. | |
| 700382742H1 | g414702 | 17 | −7 | gb105pln | H. lanatus mRNA for allergen Hol-lI. | |
| 700356427H1 | g11243 | 38 | −15 | gb105pln | Arabidopsis thaliana mRNA for mitochondrial citrate synthetase. | |
| 700355002H1 | g2662340 | 58 | −35 | gb105pln | Oryza sativa mRNA for EF-1 alpha, complete cds. | |
| 700282505H2 | g2429086 | 18 | −1 | gb105pln | Hordeum vulgare lipoxygenase 2 (LoxC) mRNA, complete cds. | |
| 700356809H1 | g20203 | 21 | −2 | gb105pln | Rice mRNA for fructose-diphosphate aldolase (EC 4.1.2.13). | |
| 700355973H1 | g172559 | 11 | −3 | gb105eukp | Sec13; Sec13p | |
| 700382885H1 | g2511690 | 16 | 6 | gb105pln | Phaseolus vulgaris Moldavian encoding cysteine proteinase precursor (clone cp41). | |
| 700382295H1 | g187338 | 26 | −20 | gb105pln | D. carota gene for elongation factor 1A. | |
| 700382768H1 | g473359 | 31 | −2 | gb105pln | P. pratense mRNA for Phl pI allergen. | |
| 700355941H1 | g20187 | 51 | −33 | gb105pln | O. sativa gene encoding calmodulin. | |
| 700353024H1 | g2565339 | 35 | −17 | gb105pln | Lupinus luteus ribosomal protein S14 (rps14) mRNA, complete cds. | |
| 700356225H1 | g217963 | 57 | −72 | gb105pln | Zea mays mRNA for rubulose 1,5-bisphosphate carboxylase small subunit, | |
| | | | | | complete cds. | |
| 7003555590H1 | g2384759 | 37 | −20 | gb105pln | Oryza sativa GDP dissociation inhibitor protein OsGDI2 (OsGDI2) mRNA, | |
| | | | | | complete cds. | |
| 700382440H1 | g157943 | 46 | −25 | gb105eukp | Mhc | |
| 700356476H1 | g22427 | 98 | −61 | gb105pln | Z. mays gene for polygalacturonase (clone 65). | |
| 700355893H1 | g2760330 | 16 | −4 | gb105eukp | F1N21.15 | |
| 700353542H1 | g2168137 | 11 | 3 | gb105eukp | PKF1 | |
| 700382424H1 | g2226414 | 18 | 7 | gb105allp | hypothetical protein | |
| 700355825H1 | g415314 | 58 | −53 | gb105pln | Rice mRNA for NADP dependent malic enzyme, complete cds. | |
| 700354945H1 | g1184775 | 70 | −35 | gb105pln | Zea mays glyceraldehyde-3-phosphate dehydrogenase GAPC4 (gpc4) mRNA, | |
| | | | | | complete cds. | |
| 700382882H1 | g454880 | 18 | 5 | gb105pln | Rice mRNA for WSI724 protein induced by water stress, complete cds. | |
| 700355711H1 | g531095 | 17 | 5 | gb105pln | Zinnia elegans mRNA for TED2, complete cds. | |
| 700355073H1 | g414737 | 48 | 7 | gb105eukp | phosphorylates 3′-OH group of adenylylsulfate; APS kinase; EC 2.7.1.25; ATP: | |
| | | | | | adenylylsulfate-3′-phosphotransferase | |
| 700355988H1 | g311658 | 17 | −8 | gb105eukp | flavonol synthase | |
| 700355960H1 | g288611 | 67 | −79 | gb105pln | Z. mays PG gene for polygalacturonase. | |
| 700382933H1 | g147142 | 5 | 7 | gb105allp | peptidase N | |
| 700282513H2 | g2313254 | 9 | −4 | gb105allp | peptide chain release factor RF-2 (prfB) | |
| 700355816H1 | g407028 | 12 | 17 | gb105pln | Zea mays gene for Ferredoxin-NADP reductase binding protein. | |
| 700355005H1 | g498772 | 59 | −49 | gb105pln | Z. mays (cv DH5xDH7) hsp70-4 mRNA for heat shock protein 70. | |
| 700356415H1 | g2267083 | 18 | 15 | gb105pln | Symbiodinium microadriaticum calmodulin (SMCaM1) mRNA, partial cds. | |
| 700352953H1 | g606810 | 35 | −6 | gb105pln | Zea mays carbonic anhydrase mRNA, complete cds. | |
| 700355160H1 | g2160158 | 22 | 4 | gb105allp | Similar to elongation factor 1-gamma (gb\|EF1G_XENLA). ESTs gb\|T20564, | |
| | | | | | gb\|T45940, gb\|T04527 come from this gene. | |
| 700354422H1 | g414907 | 26 | −32 | gb105pln | P. pratense mRNA for pollen allergen PhlpI. | |
| 700382196H1 | g973312 | 27 | −17 | gb105pln | Arabidopsis thaliana myo-inositol 1-phosphate synthase isozyme-2 mRNA, | |
| | | | | | complete cds. | |
| 700282564H2 | g939778 | 29 | −23 | gb105pln | Zea mays MADS-box protein (ZAG3) mRNA, complete cds. | |
| 700354611H1 | g1001440 | 6 | 7 | gb105allp | beta transducin-like protein | |
| 700354248H1 | g414702 | 17 | 16 | gb105pln | H. lanatus mRNA for allergen Hol-lI. | |
| 700353546H1 | g1051108 | 39 | −27 | gb105pln | A. thaliana mRNA for adenine nucleotide translocase. | |
| 700282416H2 | g287829 | 46 | −61 | gb105pln | Z. mays gene for polygalacturonase. | |
| 700354442H1 | g575354 | 54 | −34 | gb105pln | O. sativa SC34 mRNA for tumor suppressor. | |
| 700282532H2 | g2760334 | 16 | 0 | gb105eukp | F1N21.5 | |
| 700354662H1 | g1302496 | 10 | 5 | gb105allp | ORF YNR015w | |
| 700353962H1 | g312180 | 21 | −5 | gb105pln | Z. mays GapC4 gene. | |
| 700355971H1 | g974301 | 10 | −1 | gb105pln | Chlamydomonas reinhardtii ribosomal protein L41 (RPL41) mRNA, complete cds. | |
| 700353887H1 | g1173556 | 30 | −11 | gb105pln | Oryza sativa Ory s 1 mRNA, complete cds. | |
| 700353639H1 | g22425 | 82 | −71 | gb105pln | Z. mays gene for polygalacturonase (clone 47). | |
| 700282533H2 | g432606 | 53 | −42 | gb105pln | ric1 = ras-related GTP binding protein possessing GTPase activity [Oryza sativa = | |
| | | | | | rice, Yamahoushi, callus, mRNA, 955 nt]. | |
| 700282526H2 | g21399 | 39 | −9 | gb105pln | Solanum tuberosum processed actin pseudogene. | |
| 700382462H1 | g2462732 | 20 | −17 | gb105eukp | F8A5.10 | |
| 700353576H1 | g22375 | 22 | −3 | gb105eukp | ORF | |
| 700353225H1 | g439585 | 48 | −30 | gb105pln | Hordeum vulgare calreticulin (CRH1) mRNA, partial cds. | |
| 700382308H1 | g829147 | 55 | −74 | gb105pln | Z. mays gene for cyclophilin. | |
| 700355854H1 | g2388565 | 11 | −2 | gb105eukp | YUP8H12.7 | |
| 700382439H1 | g35597 | 12 | 6 | gb105allp | PR 264 | |
| 700282591H2 | g211947 | 23 | 5 | gb105allp | hydroxymethylglutaryl-CoA lyase | |
| 700355072H1 | g1666176 | 49 | −23 | gb105pln | N. tabacum mRNA for subunit of NADH:ubiquinone oxidoreductase complex I. | |
| 700354952H1 | g1215982 | 20 | 4 | gb105allp | ribosomal protein L21 | |
| 700382042H1 | g2462746 | 21 | −9 | gb105eukp | F8A5.32 | |
| 700353543H1 | g1667593 | 57 | −34 | gb105pln | Oryza sativa transmembrane protein mRNA, complete cds. | |
| 700354340H1 | g6961 | 11 | 3 | gb105eukp | ZK637.8b | |
| 700352886H1 | g17795 | 15 | 0 | gb105eukp | Bplo | |
| 700382843H1 | g509769 | 11 | 0 | gb105eukp | seed-specific low molecular weight sulfur-rich protein | |
| 700355191H1 | g1370199 | 32 | −12 | gb105pln | L. japonicus mRNA for small GTP-binding protein, RAC1. | |
| 700352666H1 | g17681 | 25 | −10 | gb105pln | A. thaliana mRNA for Wilm's tumor suppresor homlogue. | |
| 700353843H1 | g746487 | 15 | 3 | gb105eukp | F48E8.2 | |
| 700355738H1 | g17561 | 11 | 5 | gb105allp | PROTEINASE INHIBITOR | |
| 700354231H1 | g312180 | 42 | −26 | gb105pln | Z. mays GapC4 gene. | |
| 700356038H1 | g666074 | 9 | 6 | gb105allp | protein-tyrosine-phosphatase | |
| 700282510H2 | g1519252 | 22 | 4 | gb105pln | Oryza sativa GF14-d protein mRNA, complete cds. | |
| 700382707H1 | g603269 | 32 | −14 | gb105eukp | YER036C; Yer036cp | |
| 700383095H1 | g899607 | 52 | −74 | gb105pln | Zea mays polyubiquitin (MubC5) mRNA, complete cds. | |
| 700355593H1 | g1167835 | 21 | −9 | gb105pln | H. lanatus mRNA for Hol lI protein. | |
| 700352890H1 | g2218151 | 39 | −43 | gb105pln | Vigna unguiculata type IIIa membrane protein cp-wap13 mRNA, complete cds. | |
| 700355710H1 | g173231 | 28 | −15 | gb105pln | Yeast (Saccharomyces cerevisiae) ribosomal 5S RNA-binding protein (YL3) gene, 5′ cds. | |
| 700354973H1 | g598193 | 7 | −1 | gb105allp | phosphatidylinositol 4-kinase | |
| 700382428H1 | g1305524 | 20 | −21 | gb105pln | Oryza sativa Wilms' tumor-related protein QM mRNA, partial cds. | |
| 700382055H1 | g2252869 | 37 | −8 | gb105eukp | AT0ZI1 | |
| 700353231H1 | g1136574 | 39 | −40 | gb105pln | Sorghum bicolor heat shock protein 70 (hsp70) pseudogene. | |
| 700354677H1 | g2623248 | 18 | −5 | gb105eukp | sugary1; SU1 isomylase | |
| 700354878H1 | g2104535 | 22 | −11 | gb105eukp | T10M13.13; T10M13.13 | |
| 700353723H1 | g169703 | 45 | 6 | gb105allp | ATP:pyruvate phosphotransferase | |
| 700354677H1 | g758726 | 18 | −6 | gb105eukp | Sugar1; Su1p | |
| 700382391H1 | g19280 | 43 | −29 | gb105pln | L. esculentum mRNA for enolase. | |
| 700353251H1 | g1246115 | 22 | −5 | gb105pln | major allergen Pha a 1 [Phalaris aquatica = canary grass, pollen, mRNA Partial, | |
| | | | | | 1088 nt]. | |
| 700382048H1 | g288373 | 43 | −71 | gb105pln | Z. mays PGg6 gene for polygalacturonase. | |
| 700382789H1 | g22277 | 91 | −83 | gb105pln | Maize mRNA for ferritin (clone FM2). | |
| 700355956H1 | g1805306 | 25 | −10 | gb105pln | Arabidopsis thaliana flavonol synthase mRNA, complete cds. | |
| 700356409H1 | g22144 | 98 | −62 | gb105pln | Maize anaerobically regulated gene for fructose fisphosphate aldolase (EC 4.1.2.13). | |
| 700355860H1 | g170783 | 26 | −22 | gb105pln | T. aestivum ubiquitin carrier protein mRNA. | |
| 700354267H1 | g397958 | 45 | −21 | gb105pln | Z. mays mRNA for beta-amylase. | |
| 700282414H2 | g1911581 | 20 | 11 | gb105pln | Cyn d 1 = major allergen {clone 14c1 and CD1} [Cynodon dactylon = Bermuda grass, | |
| | | | | | pollen, mRNA Partial, 759 nt]. | |
| 700356288H1 | g902585 | 27 | −23 | gb105pln | Zea mays clone MubG9 ubiquitin gene, complete cds. | |
| 700353245H1 | g255568 | 30 | −15 | gb105pln | pollen specific protein [Zea mays = corn, mRNA, 943 nt]. | |
| 700355075H1 | g2224927 | 26 | −6 | gb105eukp | EIL1; ethylene-insensitive3-like1 | |
| 700356246H1 | g1236961 | 24 | −4 | gb105eukp | hydrolysis of the cyanogenic glucoside (R)-prunasin; prunasin hydrolase precursor; | |
| | | | | | EC 3.2.1.21 | |
| 700353983H1 | g2689242 | 30 | −8 | gb105pln | A. thaliana mRNA for ubiquitin conjugating enzyme UBC2. | |
| 700353573H1 | g1173556 | 45 | −11 | gb105pln | Oryza sativa Ory s 1 mRNA, complete cds. | |
| 700354984H1 | g1171161 | 36 | 5 | gb105eukp | pectate lyase homolog | |
| 700353024H1 | g463856 | 27 | −7 | gb105pln | Chlamydomonas reinhardtii 21gr ribosomal protein S14 (CRY1) gene, complete cds. | |
| 700352825H1 | g2252856 | 16 | −0 | gb105eukp | A_TM018A10.8 | |
| 700356856H1 | g22458 | 30 | −35 | gb105pln | Z. mays pollen specific mRNA C-terminal (clone 4H7). | |
| 700352982H1 | g485376 | 89 | −58 | gb105pln | Zea mays alpha-3-tubulin gene, complete cds. | |
| 700352650H1 | g1877396 | 54 | −45 | gb105pln | R. communis mRNA for shaggy-like kinase, partial. | |
| 700354681H1 | g313139 | 73 | −67 | gb105pln | Z. mays ZmPRO2 mRNA for profilin 2. | |
| 700354224H1 | g22425 | 63 | 5 | gb105pln | Z. mays gene for polygalacturonase (clone 47). | |
| 700383175H1 | g414907 | 28 | −13 | gb105pln | P. pratense mRNA for pollen allergen PhlpI. | |
| 700354513H1 | g2058454 | 45 | −2 | gb105eukp | cyclic nucleotide gated channel protein; CaMB-channel protein | |
| 700355054H1 | g406280 | 45 | −21 | gb105pln | F. trinervia ftcpt mRNA for triose phosphate/phosphate translocator. | |
| 700356468H1 | g498747 | 12 | 7 | gb105eukp | Pir7b | |
| 700355594H1 | g1872473 | 7 | 4 | gb105eukp | TA-MT; delta-24-sterol methyltransferase | |
| 700353685H1 | g2529339 | 15 | 4 | gb105pln | Zea maize AGAMOUS homologue mRNA, complete cds. | |
| 700352672H1 | g348695 | 41 | −29 | gb105pln | Catharanthus roseus heat shock protein 90 mRNA, complete cds. | |
| 700352775H1 | g22416 | 62 | −63 | gb105pln | Z. mays mRNA for polygalacturonase (clone PG1). | |
| 700355069H1 | g21399 | 37 | −3 | gb105pln | Solanum tuberosum processed actin pseudogene. | |
| 700357410H1 | g1871186 | 16 | −8 | gb105eukp | T06D20.14 | |
| 700355918H1 | g1572665 | 12 | 0 | gb105eukp | kinesin-like protein K6 | |
| 700353243H1 | g167961 | 25 | −2 | gb105pln | D. caryophyllus S-adenosylmethionine synthetase (CARSAM2) mRNA, complete cds. | |
| 700382445H1 | g396209 | 21 | −4 | gb105pln | S. polyrrhiza mRNA for D-myo-inositol-3-phosphate synthase. | |
| 700382025H1 | g166379 | 21 | −25 | gb105pln | Alfalfa glucose-regulated endoplasmic reticular protein mRNA, complete cds. | |
| 700356111H1 | g600768 | 26 | −46 | gb105pln | Oryza sativa cyclophilin 2 (Cyp2) mRNA, complete cds. | |
| 700353523H1 | g22425 | 47 | −42 | gb105pln | Z. mays gene for polygalacturonase (clone 47). | |
| 700282543H2 | g496683 | 8 | 7 | gb105eukp | YBL0838; B-130 protein | |
| 700382905H1 | g2384677 | 10 | −2 | gb105eukp | AtKT5; putative potassium transporter AtKT5p | |
| 700382166H1 | g927571 | 75 | −74 | gb105pln | Z. mays mRNA for calreticulin precursor. | |
| 700355311H1 | g929961 | 19 | −4 | gb105eukp | ribosomal protein | |
| 700382633H1 | g2992 | 19 | 4 | gb105pln | Neurospora crassa crp-1 mRNA for ribosomal protein homologous to Yeast cyH2 gene | |
| | | | | | encoding L29. | |
| 700382657H1 | g22747 | 43 | −19 | gb105pln | L. longiflorum mRNA for actin depolymerizing factor. | |
| 700357423H1 | g22427 | 54 | −64 | gb105pln | Z. mays gene for polygalacturonase (clone 65). | |
| 700355780H1 | g545715 | 40 | 7 | gb105allp | metallothionein MT-0 isoform, metallothionein-0 [human, monocytes, Peptide Partial, | |
| | | | | | 31 aa] | |
| 700383152H1 | g473604 | 53 | −43 | gb105pln | Zea mays W-22 histone H2B mRNA, complete cds. | |
| 700355338H1 | g454881 | 21 | −18 | gb105pln | Rice gene for thioredoxin h, complete cds. | |
| 700356916H1 | g248338 | 100 | −36 | gb105pln | polyubiquitin [maize, Genomic, 3439 nt]. | |
| 700382016H1 | g22416 | 40 | −84 | gb105pln | Z. mays mRNA for polygalacturonase (clone PG1). | |
| 700352650H1 | g1161511 | 54 | −45 | gb105pln | A. thaliana mRNA for shaggy-like kinase etha. | |
| 700383122H1 | g2276460 | 13 | 0 | gb105allp | multispanning membrane protein | |
| 700382389H1 | g2827140 | 30 | −23 | gb105pln | Arabidopsis thaliana cellulose synthase catalytic subunit (Ath-A) mRNA, complete cds. | |
| 700355735H1 | g1209700 | 26 | −27 | gb105pln | Zea mays ribosomal protein L12 mRNA, complete cds. | |
| 700382295H1 | g18438 | 29 | −23 | gb105pln | Euglena gracilis tef mRNA for elongation factor EF-1alpha. | |
| 700355401H1 | g409074 | 7 | 5 | gb105allp | HBp15/L22 | |
| 700382658H1 | g722327 | 64 | −82 | gb105pln | Zea mays clone Zm-Rab2-B GTP binding protein (rab2) mRNA, complete cds. | |
| 700382956H1 | g463251 | 23 | −1 | gb105pln | M. sativa (Nagyszenasi) mRNA for ribosomal protein RL5. | |
| 700355867H1 | g1707856 | 59 | −53 | gb105pln | T. aestivum mRNA for obtusifoliol 14-alpha-demethylase (CYP51), clone w516, partial. | |
| 700355760H1 | g22125 | 65 | −44 | gb105pln | Zea mays Adh2-N gene for alcohol dehydrogenase 2. | |
| 700282456H2 | g1490417 | 23 | −11 | gb105eukp | mor; global transcriptional regulation; 89B helicase | |
| 700355960H1 | g288378 | 49 | −58 | gb105pln | Z. mays gene PG for polygalacturonase. | |
| 700382057H1 | g2230757 | 18 | −11 | gb105eukp | J10; dnaJ-like protein | |
| 700357413H1 | g1236950 | 41 | −30 | gb105pln | Glycine max nucleoside diphosphate kinase mRNA, complete cds. | |
| 700354788H1 | g2464946 | 24 | −11 | gb105eukp | purple acid phosphatase | |
| 700282527H2 | g168313 | 20 | 9 | gb105pln | L. perenne pollen allergen (Lol pI) gene, 3′ end. | |
| 700355460H1 | g2673867 | 20 | 2 | gb105pln | Antirrhinum majus mRNA for fimbriata-associated protein 1, partial. | |
| 700353620H1 | g1575129 | 89 | −86 | gb105pln | Zea mays lumenal binding protein cBiPe3 mRNA, complete cds. | |
| 700382320H1 | g2656025 | 47 | −39 | gb105pln | Arabidopsis thaliana genomic DNA, chromosome 5, P1 clone: MCD7. | |
| 700382310H1 | g1911581 | 25 | −20 | gb105pln | Cyn d 1 = major allergen {clone 14c1 and CD1} [pu Cynodon dactylon = Bermuda | |
| | | | | | grass, pollen, mRNA Partial, 759 nt]. | |
| 700382269H1 | g170205 | 24 | −23 | gb105pln | N. plumbaginifolia H+-translocating ATPase mRNA. | |
| 700382993H1 | g2330862 | 16 | −4 | gb105eukp | SPAC6F12.16; hypothetical helicase | |
| 700355541H1 | g433038 | 14 | 16 | gb105pln | Zea mays W-22 clone cDNA7 retroelement PREM-1-containing mRNA. | |
| 700353138H1 | g22416 | 46 | −61 | gb105pln | Z. mays mRNA for polygalacturonase (clone PG1). | |
| 700383090H1 | g625108 | 48 | −8 | gb105eukp | YLR388W; Ylr388wp | |
| 700353247H1 | g2062169 | 13 | 2 | gb105eukp | T02O04.17; ABC transporter (PDR5-like) isolog | |
| 700352845H1 | g1841357 | 12 | 2 | gb105eukp | rps11; mitochondrial ribosomal protein S11 (nuclear encoded) | |
| 700382339H1 | g669002 | 55 | −47 | gb105pln | Glycine max calnexin mRNA, complete cds. | |
| 700353822H1 | g2618599 | 21 | −15 | gb105pln | Arabidopsis thaliana genomic DNA, chromosome 5, P1 clone: MBD2, complete | |
| | | | | | sequence. | |
| 700353796H1 | g2244780 | 34 | −10 | gb105eukp | hypothetical protein | |
| 700382264H1 | g2842482 | 10 | 6 | gb105eukp | F21O9.80; protein phosphatase 2C-like protein | |
| 700355941H1 | g310314 | 51 | −33 | gb105pln | Oryza sativa calmodulin gene, complete cds. | |
| 700383085H1 | g22458 | 59 | −61 | gb105pln | Z. mays pollen specific mRNA C-terminal (clone 4H7). | |
| 700357427H1 | g551482 | 85 | −2 | gb105pln | Zea mays ABA- and ripening-inducible-like protein mRNA, complete cds. | |
| 700357183H1 | g2584827 | 10 | 16 | gb105pln | Sequence of BAC F12F1 from Arabidopsis thaliana chromosome 1, complete sequence. | |
| 700355484H1 | g20163 | 30 | −16 | gb105pln | O. sativa Rrl5 mRNA for 5S ribosomal RNA. | |
| 700354265H1 | g1354865 | 24 | −4 | gb105eukp | COI; cytochrome oxidase | |
| 700353030H1 | g414702 | 41 | −4 | gb105pln | H. lanatus mRNA for allergen Hol-lI. | |
| 700356880H1 | g287829 | 96 | −21 | gb105pln | Z. mays gene for polygalacturonase. | |
| 700353138H1 | g288366 | 47 | −63 | gb105pln | Z. mays PGg14 gene for polygalacturonase. | |
| 700382763H1 | g1041240 | 88 | −43 | gb105allp | gamma-adducin | |
| 700354776H1 | g30295 | 10 | −1 | gb105allp | cytochrome c oxidase subunit VIb (AA 1-86) | |
| 700282415H2 | g1899188 | 24 | −8 | gb105eukp | DNA binding protein ACBF | |
| 700382777H1 | g313137 | 33 | −81 | gb105pln | Z. mays ZmPRO1 mRNA for profilin 1. | |
| 700383073H1 | g968901 | 12 | −2 | gb105pn | Rice mRNA for ribosomal protein S8, complete cds. | |
| 700382282H1 | g533692 | 18 | −3 | gb105eukp | protease inhibitor | |
| 700353854H1 | g414702 | 45 | −30 | gb105pln | H. lanatus mRNA for allergen Hol-lI. | |
| 700352862H1 | g1890352 | 27 | 5 | gb105eukp | at12; transcription factor L2 | |
| 700355935H1 | g248336 | 75 | −75 | gb105pln | polyubiquitin [maize, Genomic, 3841 nt]. | |
| 700382016H1 | g22421 | 40 | −84 | gb105pln | Z. mays mRNA for polygalacturonase (clone PG3). | |
| 700356796H1 | g454880 | 25 | 14 | gb105pln | Rice mRNA for WSI724 protein induced by water stress, complete cds. | |
| 700355192H1 | g22651 | 49 | −57 | gb105pln | H. vulgare mRNA for GRP94 homologue. | |
| 700355409H1 | g1890352 | 11 | 4 | gb105eukp | at12; transcription factor L2 | |
| 700355052H1 | g1399512 | 20 | −1 | gb105eukp | repE; repE | |
| 700382310H1 | g19495 | 30 | −20 | gb105pln | L. perenne RNA for Lol pI gene. | |
| 700353643H1 | g484043 | 8 | 6 | gb105allp | ribosomal protein S12 | |
| 700352771H1 | g1553130 | 38 | −30 | gb105pln | Gossypium hirsutum ribosomal protein L44 isoform b (RL44), complete cds. | |
| 700382663H1 | g2435514 | 17 | −17 | gb105eukp | A_TM017A05.2 | |
| 700352754H1 | g1707854 | 62 | −46 | gb105pln | T. aestivum mRNA for obtusifoliol 14-alpha-demethylase (CYP51), clone w51, partial. | |
| 700382924H1 | g2317909 | 12 | 1 | gb105allp | CER1-like protein | |
| 700382825H1 | g5271 | 24 | −9 | gb105pln | Yeast (Saccharomyces cerevisiae) p68 gene. | |
| 700353820H1 | g20238 | 22 | 3 | gb105eukp | GOS2 | |
| 700282505H2 | g2182266 | 27 | −18 | gb105pln | Hordeum vulgare lipoxygenase (LoxB) mRNA, complete cds. | |
| 700354704H1 | g5056 | 19 | −6 | gb105pln | Yeast (Saccharomyces pombe) rpgL29 gene for ribosomal protein L29. | |
| 700353435H1 | g169785 | 43 | −5 | gb105pln | Rice cytochrome c (OsCc-1) gene, complete cds. | |
| 700353164H1 | g168396 | 12 | 16 | gb105pln | Zea mays auxin-binding protein (abp1) gene, exons 1-5 and complete cds. | |
| 700382023H1 | g218160 | 37 | −22 | gb105pln | Oryza sativa mRNA for elongation factor 1 beta′. | |
| 700355437H1 | g886430 | 19 | −8 | gb105eukp | P2; putative NADPH oxidoreductase; zeta-crystallin homologue | |
| 700354841H1 | g531057 | 26 | −21 | gb105pln | Wheat mRNA for protein H2B-8, complete cds. | |
| 700382302H1 | g1532167 | 26 | −4 | gb105eukp | AT.I.24-5 | |
| 700354973H1 | g1477958 | 7 | −1 | gb105allp | type 3 phosphatidylinositol 4-kinase, type 3 PtdIns kinase [cattle, brain, Peptide Partial, | |
| | | | | | 1466 aa] | |
| 700382742H1 | g414907 | 16 | −7 | gb105pln | P. pratense mRNA for pollen allergen PhlpI. | |
| 700382673H1 | g156372 | 27 | −14 | gb105eukp | mlc-1; myosin light chain 1 | |
| 700382325H1 | g2429289 | 13 | −1 | gb105pln | Oryza sativa peroxidase (POX5.1) mRNA, complete cds. | |
| 700355226H1 | g414702 | 17 | 3 | gb105pln | H. lanatus mRNA for allergen Hol-lI. | |
| 700382634H1 | g168665 | 48 | −57 | gb105pln | Maize 16-kDa zein-2 mRNA, complete cds. | |
| 700356427H1 | g483509 | 38 | −15 | gb105pln | S. tuberosum mRNA for mitochondrial citrate-synthase. | |
| 700356969H1 | g313141 | 59 | −33 | gb105pln | Z. mays ZmPRO3 mRNA for profilin 3. | |
| 700354490H1 | g1399266 | 18 | −12 | gb105pln | Arabidopsis thaliana calmodulin-domain protein kinase CDPK isoform 4 (CPK4) | |
| | | | | | mRNA, partial cds. | |
| 700353025H1 | g1519252 | 50 | −52 | gb105pln | Oryza sativa GF14-d protein mRNA, complete cds. | |
| 700352603H1 | g899607 | 93 | −83 | gb105pln | Zea mays polyubiquitin (MubC5) mRNA, complete cds. | |
| 700382843H1 | g16427 | 14 | 3 | gb105eukp | protease inhibitor II | |
| 700282411H2 | g1167835 | 27 | −10 | gb105pln | H. lanatus mRNA for Hol lI protein. | |
| 700382119H1 | g16210 | 18 | 4 | gb105pln | Arabidopsis thaliana calnexin homolog. | |
| 700382023H1 | g218340 | 37 | −23 | gb105pln | Triticum aestivum mRNA for elongation factor 1 beta′. | |
| 700382314H1 | g1184775 | 64 | −48 | gb105pln | Zea mays glyceraldehyde-3-phosphate dehydrogenase GAPC4 (gpc4) mRNA, | |
| | | | | | complete cds. | |
| 700355818H1 | g293901 | 69 | −76 | gb105pln | Zea mays Zea mI gene, complete cds. | |
| 700354594H1 | g17917 | 18 | 6 | gb105eukp | S-receptor kinase related protein | |
| 700352620H1 | g1777706 | 97 | −81 | gb105pln | Zea mays 18S ribosomal RNA gene, partial sequence. | |
| 700382272H1 | g20373 | 49 | −58 | gb105pln | O. sativa mRNA for sucrose synthase. | |
| 700353275H1 | g255569 | 12 | −0 | gb105eukp | pollen specific protein | |
| 700355896H1 | g473359 | 27 | −8 | gb105pln | P. pratense mRNA for Phl pI allergen. | |
| 700352780H1 | g1890351 | 22 | −1 | gb105pln | A. thaliana mRNA for transcription factor L2. | |
| 700353917H1 | g22418 | 64 | 1 | gb105pln | Z. mays gene for polygalacturonase (clone 17). | |
| 700353887H1 | g1911581 | 27 | −10 | gb105pln | Cyn d 1 = major allergen {clone 14c1 and CD1} [Cynodon dactylon = Bermuda grass, | |
| | | | | | pollen, mRNA Partial, 759 nt]. | |
| 700356564H1 | g20323 | 45 | −48 | gb105pln | O. sativa RAc1 gene for actin. | |
| 700282422H2 | g168313 | 18 | 10 | gb105pln | L. perenne pollen allergen (Lol pI) gene, 3′ end. | |
| 700354947H1 | g549983 | 79 | −17 | gb105pln | Pennisetum ciliare possible apospory-associated mRNA clone pSUB C, complete cds. | |
| 700353291H1 | g1395186 | 19 | 4 | gb105allp | proteasomal ATPase (Tat-binding protein7) | |
| 700352841H1 | g2739435 | 12 | 6 | gb105allp | (p)ppGpp synthetase | |
| 700382268H1 | g2160144 | 5 | 6 | gb105allp | Strong similarity to Arabidopsis oligopeptide transporter (gb\|X77503). | |
| 700353168H1 | g1184773 | 46 | −48 | gb105pln | Zea mays glyceraldehyde-3-phosphate dehydrogenase GAPC3 (gpc3) mRNA, | |
| | | | | | complete cds. | |
| 700352809H1 | g18138 | 16 | −3 | gb105pln | C. roseus cpr mRNA for NADPH-ferrihemoprotein reductase. | |
| 700354644H1 | g1184775 | 89 | −72 | gb105pln | Zea mays glyceraldehyde-3-phosphate dehydrogenase GAPC4 (gpc4) mRNA, | |
| | | | | | complete cds. | |
| 700353568H1 | g1814407 | 36 | 4 | gb105eukp | PMA; plasma membrane proton pump; H+-transporting ATPase; EC 3.6.1.35 | |
| 700356143H1 | g20310 | 13 | 6 | gb105eukp | pollen specific gene | |
| 700355296H1 | g1314155 | 43 | 4 | gb105allp | unknown | |
| 700357106H1 | g1778820 | 77 | 4 | gb105pln | Oryza sativa S-adenosyl-L-methionine synthetase (pOS-SAMS2) mRNA, complete cds. | |
| 700382359H1 | g2398828 | 31 | −9 | gb105pln | Solanum tuberosum mRNA for mitochondrial uncoupling protein. | |
| 700382209H1 | g2384757 | 37 | −15 | gb105pln | Oryza sativa GDP dissociation inhibitor protein OsGDI1 (OsGDI1) mRNA, | |
| | | | | | complete cds. | |
| 700356985H1 | g506533 | 51 | −11 | gb105pln | N. tabacum PKTL7 mRNA for protein kinase. | |
| 700355958H1 | g1498391 | 46 | −71 | gb105pln | Zea mays actin (Maz65) gene, partial cds. | |
| 700353256H1 | g2654009 | 39 | −16 | gb105eukp | Rop4; Rho-like GTP binding protein | |
| 700353074H1 | g434329 | 63 | −63 | gb105pln | Z. mays mRNA gs1-4 for glutamine synthetase. | |
| 700282471H2 | g166813 | 12 | 6 | gb105eukp | pro25; serine threonine kinase | |
| 700382266H1 | g511665 | 33 | −32 | gb105pln | Rice gene for aspartic protease, complete cds. | |
| 700355582H1 | g1616612 | 18 | −8 | gb105eukp | small GTP-binding protein | |
| 700382196H1 | g396209 | 42 | −35 | gb105pln | S. polyrrhiza mRNA for D-myo-inositol-3-phosphate synthase. | |
| 700354874H1 | g902583 | 33 | −49 | gb105pln | Zea mays clone MubG1 ubiquitin gene, complete cds. | |
| 700382753H1 | g2360945 | 13 | 4 | gb105allp | 34 kDa Mov34 isologue | |
| 700354317H1 | g2342495 | 18 | 1 | gb105pln | Ananas comosus mRNA for bromelain, complete cds. | |
| 700353132H1 | g1399270 | 19 | 1 | gb105pln | Arabidopsis thaliana calmodulin-domain protein kinase CDPK isoform 2 (CPK2) | |
| | | | | | mRNA, complete cds. | |
| 700382393H1 | g487286 | 44 | 1 | gb105pln | Rice mRNA EN053, partial sequence. | |
| 700356038H1 | g167862 | 11 | 5 | gb105allp | protein tyrosine phosphatase | |
| 700355064H1 | g1519252 | 32 | −33 | gb105pln | Oryza sativa GF14-d protein mRNA, complete cds. | |
| 700355719H1 | g1049441 | 14 | 6 | gb105allp | similar to B. subtilis glycerol-3-phosphate cytidylyltransferase (SP:TAGD_BACSU, | |
| | | | | | P27623) | |
| 700354543H1 | g1556430 | 50 | −11 | gb105pln | Populus hybrid mRNA for citrate synthase. | |
| 700353835H1 | g1532047 | 37 | −26 | gb105pln | O. sativa mRNA for S-adenosylmethionine decarboxylase. | |
| 700352838H1 | g2252856 | 27 | 0 | gb105eukp | A_TM018A10.8 | |
| 700382315H1 | g499011 | 79 | −71 | gb105pln | S. vulgare SoAc1 mRNA | |
| 700354660H1 | g2529657 | 15 | 2 | gb105pln | Arabidopsis thaliana chromosome II BAC T30B22 genomic sequence, complete | |
| | | | | | sequence. | |
| 700382741H1 | g1661134 | 26 | −1 | gb105allp | heat shock 70 protein | |
| 700354379H1 | g1185553 | 36 | −54 | gb105pln | Zea mays glyceraldehyde-3-phosphate dehydrogenase (gpc2) gene, complete cds. | |
| 700355245H1 | g397958 | 97 | −85 | gb105pln | Z. mays mRNA for beta-amylase. | |
| 700355696H1 | g22426 | 11 | 8 | gb105allp | polygalacturonase | |
| 700356086H1 | g22375 | 9 | 2 | gb105eukp | ORF | |
| 700382104H1 | g1293846 | 9 | 5 | gb105eukp | C42D8.3 | |
| 700356196H1 | g2446980 | 32 | −9 | gb105pln | Arabidopsis thaliana mRNA for AtGDI2, complete cds. | |
| 700382061H1 | g1815759 | 14 | 7 | gb105allp | pollen-specific protein | |
| 700355541H1 | g2832242 | 14 | 16 | gb105pln | Zea mays 22-kDa alpha zein gene cluster, complete sequence. | |
| 700353022H1 | g1246115 | 26 | −8 | gb105pln | major allergen Pha a 1 [Phalaris aquatica = canary grass, pollen, mRNA Partial, | |
| | | | | | 1088 nt]. | |
| 700382952H1 | g313139 | 30 | −48 | gb105pln | Z. mays ZmPRO2 mRNA for profilin 2. | |
| 700353963H1 | g288378 | 81 | −12 | gb105pln | Z. mays gene PG for polygalacturonase. | |
| 700352605H1 | g1825459 | 7 | 4 | gb105allp | Dac gIII allergen | |
| 700354271H1 | g22428 | 16 | −0 | gb105eukp | polygalacturonase | |
| 700353569H1 | g395071 | 34 | −15 | gb105pln | V. faba guanine nucleotide regulatory protein mRNA, complete CDS. | |
| 700352887H1 | g790478 | 58 | −12 | gb105pln | N. tabacum mRNA for inorganic pyrophosphatase (TVP9 clone). | |
| 700353243H1 | g450548 | 34 | −13 | gb105pln | O. sativa (pRSAM-1) gene for S-adenosyl methionine synthetase. | |
| 700382661H1 | g171091 | 16 | 7 | gb105eukp | ASF1; derepression of silent loci when overexpressed | |
| 700354663H1 | g21955 | 22 | 2 | gb105eukp | beta-glucosidase; EC 3.2.1.21 | |
| 700383086H1 | g1814400 | 37 | −31 | gb105pln | Mesembryanthemum crystallinum phosphoglucomutase (PGM) mRNA, complete cds. | |
| 700382053H1 | g1370470 | 9 | 5 | gb105eukp | ALG5 | |
| 700355229H1 | g288366 | 74 | −71 | gb105pln | Z. mays PGg14 gene for polygalacturonase. | |
| 700353609H1 | g1668772 | 23 | 15 | gb105pln | O. sativa mRNA for SMT3 protein. | |
| 700352769H1 | g606810 | 38 | −19 | gb105pln | Zea mays carbonic anhydrase mRNA, complete cds. | |
| 700355519H1 | g499011 | 35 | −47 | gb105pln | S. vulgare SoAc1 mRNA. | |
| 700353573H1 | g1911581 | 37 | −5 | gb105pln | Cyn d 1 = major allergen {clone 14c1 and CD1} [Cynodon dactylon = Bermuda grass, | |
| | | | | | pollen, mRNA Partial, 759 nt]. | |
| 700357183H1 | g2781363 | 23 | −9 | gb105eukp | F24O1.19 | |
| 700356927H1 | g498040 | 36 | −4 | gb105eukp | ORF | |
| 700282414H2 | g414907 | 19 | 13 | gb105pln | P. pratense mRNA for pollen allergen PhlpI. | |
| 700382661H1 | g1008304 | 16 | 7 | gb105allp | ORF YJL115w | |
| 700355765H1 | g295850 | 83 | 4 | gb105eukp | fructose bisphosphate aldolase | |
| 700353834H1 | g414702 | 16 | 5 | gb105pln | H. lanatus mRNA for allergen Hol-lI. | |
| 700352776H1 | g899607 | 52 | −48 | gb105pln | Zea mays polyubiquitin (MubC5) mRNA, complete cds. | |
| 700356595H1 | g22427 | 85 | −56 | gb105pln | Z. mays gene for polygalacturonase (clone 65). | |
| 700382448H1 | g20255 | 34 | −49 | gb105pln | O. sativa gene for heat shock protein 82 HSP82. | |
| 700382440H1 | g157944 | 46 | −25 | gb105eukp | Mhc | |
| 700382017H1 | g2264313 | 16 | −11 | gb105pln | Arabidopsis thaliana genomic DNA,chromosome 5, P1 clone: MOP10, complete | |
| | | | | | sequence. | |
| 700354325H1 | g1498385 | 45 | −55 | gb105pln | Zea mays actin (Maz87) gene, partial cds. | |
| 700357441H1 | g473359 | 22 | −4 | gb105pln | P. pratense mRNA for Phl pI allergen. | |
| 700355064H1 | g22606 | 24 | −10 | gb105pln | H. vulgare mRNA for 14-3-3 protein homologue (14-3-3a). | |
| 700353025H1 | g1519248 | 32 | −30 | gb105pln | Oryza sativa GF14-b protein mRNA, complete cds. | |
| 700382283H1 | g871510 | 15 | 7 | gb105eukp | small GTP-binding protein | |
| 700355711H1 | g531096 | 26 | −4 | gb105eukp | TED2 | |
| 700356537H1 | g538427 | 40 | 10 | gb105pln | Oryza sativa ribosomal protein S16 mRNA, complete cds. | |
| 700353891H1 | g28547 | 30 | −22 | gb105allp | alpha globin | |
| 700356584H1 | g22458 | 98 | −23 | gb105pln | Z. mays pollen specific mRNA C-terminal (clone 4H7). | |
| 700355433H1 | g2275016 | 17 | −7 | gb105allp | elastase | |
| 700382127H1 | g16210 | 28 | −7 | gb105pln | Arabidopsis thaliana calnexin homolog. | |
| 700382157H1 | g1184775 | 79 | −60 | gb105pln | Zea mays glyceraldehyde-3-phosphate dehydrogenase GAPC4 (gpc4) mRNA, | |
| | | | | | complete cds. | |
| 700356567H1 | g473359 | 42 | −12 | gb105pln | P. pratense mRNA for Phl pI allergen. | |
| 700356413H1 | g22484 | 61 | −46 | gb105pln | Z. mays RNA for superoxide dismutase Sod4A. | |
| 700355842H1 | g2209343 | 9 | 2 | gb105allp | copper transport protein Atox1 | |
| 700357168H1 | g288378 | 86 | −92 | gb105pln | Z. mays gene PG for polygalacturonase. | |
| 700354380H1 | g487291 | 27 | −5 | gb105pln | Rice mRNA EN171, partial sequence. | |
| 700355583H1 | g1477683 | 25 | −10 | gb105pln | Oryza sativa SNF1-related protein kinase (RSk1) mRNA, complete cds. | |
| 700353120H1 | g1008444 | 36 | −26 | gb105pln | T. aestivum mRNA for profilin (clone TaPRO3). | |
| 700354805H1 | g1906825 | 9 | 15 | gb105pln | A. thaliana hsp81.2 gene. | |
| 700355825H1 | g168527 | 63 | −57 | gb105pln | Maize NADP-dependent malic enzyme (Me1) mRNA, complete cds. | |
| 700382327H1 | g1655482 | 15 | −1 | gb105eukp | delta subunit of mitochondrial F1-ATPase | |
| 700382068H1 | g2160172 | 12 | −1 | gb105eukp | F21M12.20 | |
| 700354550H1 | g166548 | 40 | −0 | gb105pln | Avena sativa vacuolar H+-ATPase 16 kDa proteolipid subunit (vatp-P1) mRNA, | |
| | | | | | complete cds. | |
| 700382850H1 | g2583106 | 25 | −5 | gb105pln | Arabidopsis thaliana chromosome II BAC F4L23 genomic sequence, complete sequence. | |
| 700354577H1 | g290054 | 40 | −1 | gb105pln | Dictyostelium discoideum HIV1 TAT-binding protein homologous protein mRNA, | |
| | | | | | complete cds. | |
| 700356969H1 | g313137 | 40 | 1 | gb105pln | Z. mays ZmPRO1 mRNA for profilin 1. | |
| 700382076H1 | g1769897 | 8 | 6 | gb105eukp | lectin receptor kinase | |
| 700382640H1 | g984697 | 27 | −14 | gb105pln | Yeast (Saccharomyces pombe) chromosome I cosmid c24H6. | |
| 700382181H1 | g599624 | 15 | −24 | gb105pln | A. thaliana mRNA for aconitase (ZAPII). | |
| 700354958H1 | g1154858 | 51 | −45 | gb105pln | H. vulgare mRNA for L24 ribosomal protein. | |
| 700353054H1 | g508502 | 11 | 2 | gb105allp | sulfite oxidase | |
| 700382655H1 | g1718236 | 11 | 0 | gb105allp | (1,4)-beta-xylan endohydrolase, isoenzyme X-I | |
| 700353278H1 | g414702 | 23 | −5 | gb105pln | H. lanatus mRNA for allergen Hol-lI. | |
| 700382920H1 | g288373 | 28 | −58 | gb105pln | Z. mays PGg6 gene for polygalacturonase. | |
| 700354972H1 | g2258468 | 35 | −2 | gb105pln | Oryza sativa replication protein A1 (Os-RPA1) mRNA, complete cds. | |
| 700355542H1 | g433044 | 19 | −30 | gb105pln | Zea mays W-22 clone PREM-1D retroelement PREM-1, partial sequence. | |
| 700355064H1 | g1519248 | 26 | −12 | gb105pln | Oryza sativa GF14-b protein mRNA, complete cds. | |
| 700383022H1 | g1246115 | 22 | −15 | gb105pln | major allergen Pha a 1 [Phalaris aquatica = canary grass, pollen, mRNA Partial, | |
| | | | | | 1088 nt]. | |
| 700382985H1 | g599992 | 9 | 6 | gb105eukp | KGD1; 2-oxoglutarate dehydrogenase E1 component | |
| 700383135H1 | g2224911 | 28 | −13 | gb105eukp | somatic embryogenesis receptor-like kinase | |
| 700382605H1 | g1825459 | 4 | 7 | gb105eukp | Dac gIII allergen | |
| 700383167H1 | g435174 | 28 | −5 | gb105pln | A. sativa (Pewi) ASTCP-K36 mRNA for t complex polypeptide 1. | |
| 700353435H1 | g218240 | 47 | −5 | gb105pln | Rice mRNA for cytochrome C (T76 gene), partial sequence. | |
| 700382880H1 | g22420 | 62 | −37 | gb105pln | Z. mays mRNA for polygalacturonase (clone PG2). | |
| 700382628H1 | g1145694 | 41 | −41 | gb105pln | Arabidopsis thaliana actin (ACT3) gene, complete cds. | |
| 700357138H1 | g288611 | 91 | −87 | gb105pln | Z. mays PG gene for polygalacturonase. | |
| 700382311H1 | g216632 | 11 | 4 | gb105allp | ORF2 | |
| 700356964H1 | g1293668 | 44 | 4 | gb105eukp | ARAC5; GTP-binding protein | |
| 700382269H1 | g218178 | 45 | −51 | gb105pln | Rice OSA1 gene for H+-ATPase, complete cds. | |
| 700382790H1 | g22416 | 68 | −59 | gb105pln | Z. mays mRNA for polygalacturonase (clone PG1). | |
| 700355010H1 | g22149 | 98 | −60 | gb105pln | Z. mays mRNA for alpha-tubulin 3. | |
| 700356258H1 | g1575127 | 81 | −79 | gb105pln | Zea mays lumenal binding protein cBiPe2 mRNA, complete cds. | |
| 700353789H1 | g288611 | 34 | −46 | gb105pln | Z. mays PG gene for polygalacturonase. | |
| 700353608H1 | g2414636 | 23 | −10 | gb105eukp | SPAC3A11.08; hypothetical protein | |
| 700352717H1 | g1161339 | 5 | 7 | gb105eukp | ERG25; C-4 sterol methyl oxidase | |
| 700352613H1 | g177216 | 17 | 8 | gb105allp | 4F2 heavy chain antigen | |
| 700356032H1 | g2645166 | 28 | −10 | gb105pln | Oryza sativa mRNA, similar to ubiquitin conjugating enzyme. | |
| 700352639H1 | g2352924 | 22 | 4 | gb105pln | Petroselinum crispum plastidic 3-deoxy-D-arabino-heptulosonate 7-phosphate synthase 2 | |
| | | | | | (pDAHPS2) mRNA, nuclear gene encoding plastid protein, complete cds. | |
| 700383050H1 | g21233 | 20 | −23 | gb105pln | S. oleracea AHRI mRNA for acetohydroxy acid reductoisomerase. | |
| 700353943H1 | g587563 | 30 | −15 | gb105pln | S. tuberosum mRNA for beta-II MPP. | |
| 700383165H1 | g339771 | 15 | −0 | gb105allp | ORF2 contains a reverse transcriptase domain.; ORF2 | |
| 700382486H1 | g303843 | 57 | −58 | gb105pln | Rice mRNA for eukaryotic initiation factor 4A, complete cds. | |
| 700382148H1 | g790969 | 52 | 5 | gb105pln | Rice mRNA for aldolase C-1, complete cds. | |
| 700354555H1 | g416256 | 34 | 6 | gb105pln | Rice mRNA for H+-ATPase, partial sequence. | |
| 700282585H2 | g168313 | 21 | −3 | gb105pln | L. perenne pollen allergen (Lol pI) gene, 3′ end. | |
| 700356574H1 | g22427 | 67 | −79 | gb105pln | Z. mays gene for polygalacturonase (clone 65). | |
| 700356235H1 | g1895091 | 18 | −2 | gb105eukp | ARG7; acetylornithine acetyltransferase | |
| 700355627H1 | g248336 | 18 | −5 | gb105pln | polyubiquitin [maize, Genomic, 3841 nt]. | |
| 700354905H1 | g644491 | 55 | −64 | gb105pln | Corn mRNA for elongation factor 1A. | |
| 700382770H1 | g559921 | 22 | 1 | gb105eukp | axi 1 | |
| 700382937H1 | g531031 | 39 | −35 | gb105pln | Pennsisetum ciliare apomixis-associated mRNA. | |
| 700355778H1 | g1839188 | 38 | −9 | gb105eukp | RHD3; putative GTP-binding protein; root hair defective 3 | |
| 700352994H1 | g290275 | 77 | −3 | gb105eukp | M(3)95A; multifunctional activity and location; ribosomal protein S3/AP endonuclease | |
| | | | | | DNA repair protein | |
| 700383176H1 | g21492 | 28 | −13 | gb105pln | S. tuberosum mRNA for mitochondrial processing peptidase. | |
| 700354351H1 | g32709 | 16 | 2 | gb105allp | IFP53 | |
| 700354351H1 | g295582 | 30 | −2 | gb105eukp | YME1; causes an increased escape of DNA from mitochondria; putative ATPase; Yme1p | |
| 700382446H1 | g2459435 | 19 | −11 | gb105eukp | F4P9.30; putative serine carboxypeptidase | |
| 700356289H1 | g2244806 | 9 | 3 | gb105eukp | hypothetical protein | |
| 700382985H1 | g763221 | 9 | 6 | gb105eukp | KGD1; 2-oxoglutarate dehydrogenase | |
| 700356188H1 | g170225 | 13 | 2 | gb105eukp | DAHPS-1; 3-deoxy-D-arabino-heptulosonate 7-phosphate synthaase; EC 4.1.2.15 | |
| 700357426H1 | g1486425 | 6 | 3 | gb105allp | BioA homologue | |
| 700354467H1 | g218142 | 13 | −24 | gb105pln | Rice mRNA for aspartic proteinase, complete cds. | |
| 700353233H1 | g288611 | 71 | −73 | gb105pln | Z. mays PG gene for polygalacturonase. | |
| 700355242H1 | g556672 | 33 | −19 | gb105pln | S. cereale (Halo) chloroplast mRNA for heat-shock protein. | |
| 700354917H1 | g22416 | 98 | −63 | gb105pln | Z. mays mRNA for polygalacturonase (clone PG1). | |
| 700353030H1 | g414907 | 42 | −5 | gb105pln | P. pratense mRNA for pollen allergen PhlpI. | |
| 700354615H1 | g2766712 | 20 | 0 | gb105pln | Arabidopsis thaliana ecotype Kl-0 chromomethylase (CMT1) mRNA, complete cds. | |
| 700352989H1 | g22416 | 72 | −56 | gb105pln | Z. mays mRNA for polygalacturonase (clone PG1). | |
| 700356055H1 | g288378 | 50 | −62 | gb105pln | Z. mays gene PG for polygalacturonase. | |
| 700352712H1 | g600768 | 28 | −27 | gb105pln | Oryza sativa cyclophilin 2 (Cyp2) mRNA, complete cds. | |
| 700353275H1 | g1815759 | 15 | −2 | gb105eukp | pollen-specific protein | |
| 700382435H1 | g287829 | 78 | −4 | gb105pln | Z. mays gene for polygalacturonase. | |
| 700353854H1 | g414907 | 45 | −29 | gb105pln | P. pratense mRNA for pollen allergen PhlpI. | |
| 700382737H1 | g313137 | 55 | −80 | gb105pln | Z. mays ZmPRO1 mRNA for profilin 1. | |
| 700356169H1 | g288611 | 71 | −78 | gb105pln | Z. mays PG gene for polygalacturonase. | |
| 700354891H1 | g2226414 | 16 | 6 | gb105eukp | SPBC31F10.03; hypothetical protein | |
| 700353794H1 | g313137 | 26 | −18 | gb105pln | Z. mays ZmPRO1 mRNA for profilin 1. | |
| 700382168H1 | g1399567 | 41 | −56 | gb105pln | Podophyllum peltatum nuclear 26S ribosomal RNA gene, partial sequence. | |
| 700357491H1 | g1707363 | 47 | −12 | gb105pln | A. thaliana mRNA for AATP2. | |
| 700383176H1 | g2062153 | 14 | 5 | gb105pln | Arabidopsis thaliana chromosome III BAC T02O04 genomic sequence, complete | |
| | | | | | sequence. | |
| 700382826H1 | g1777706 | 57 | −63 | gb105pln | Zea mays 18S ribosomal RNA gene, partial sequence. | |
| 700356779H1 | g2190543 | 24 | 3 | gb105eukp | F5I14.6 | |
| 700356442H1 | g288373 | 98 | −59 | gb105pln | Z. mays PGg6 gene for polygalacturonase. | |
| 700355589H1 | g2065531 | 29 | −27 | gb105eukp | Cel3; endo-1,4-beta-glucanase; EC 3.2.1.4 | |
| 700356190H1 | g169989 | 18 | 5 | gb105allp | NADPH-specific isocitrate dehydrogenase | |
| 700352627H1 | g2673901 | 19 | 7 | gb105pln | Arabidopsis thaliana chromosome II BAC T24P15 genomic sequence, complete | |
| | | | | | sequence. | |
| 700382044H1 | g22427 | 90 | −85 | gb105pln | Z. mays gene for polygalacturonase (clone 65). | |
| 700356122H1 | g402903 | 17 | −8 | gb105pln | Arabidopsis thaliana Columbia laminin receptor-like protein mRNA, complete cds. | |
| 700383095H1 | g777757 | 43 | −57 | gb105pln | Saccharum hybrid (clone SCUBI561) polyubiquitin mRNA, complete cds. | |
| 700353633H1 | g2654869 | 23 | −2 | gb105pln | Oryza sativa RbohAOsp mRNA, partial cds. | |
| 700355921H1 | g2281115 | 27 | −16 | gb105eukp | T10P11.14.1; putative cullin-like 1 protein | |
| 700352605H1 | g415896 | 8 | 3 | gb105eukp | PHL PII, pollen allergen | |
| 700355191H1 | g2645642 | 32 | −12 | gb105pln | Arabidopsis thaliana Rho-like GTP binding protein (Rop6) mRNA, complete cds. | |
| 700354776H1 | g2098574 | 10 | −1 | gb105allp | COXG | |
| 700353016H1 | g168460 | 76 | −7 | gb105pln | Zea mays cyclophilin (CyP) mRNA, complete cds. | |
| 700355563H1 | g414703 | 16 | −3 | gb105eukp | allergen Hol-lI | |
| 700353045H1 | g22747 | 27 | −30 | gb105pln | L. longiflorum mRNA for actin depolymerizing factor. | |
| 700382868H1 | g561923 | 16 | −11 | gb105eukp | M01F1.2 | |
| 700357407H1 | g1667262 | 22 | −8 | gb105eukp | B0035.1 | |
| 700353690H1 | g551289 | 34 | −53 | gb105pln | Z. mays (W22) phosphoglycerate mutase gene exons 2-8. | |
| 700355824H1 | g5230 | 13 | 7 | gb105pln | U. maydis encoding iron-sulphur subunit of succinate dehydrogenase. | |
| 700355732H1 | g2224901 | 28 | −5 | gb105eukp | PcMYB1; DNA binding protein; PcMYB1 protein | |
| 700282456H2 | g171965 | 26 | −18 | gb105eukp | MOT1; Mot1 | |
| 700356115H1 | g288373 | 98 | −27 | gb105pln | Z. mays PGg6 gene for polygalacturonase. | |
| 700355163H1 | g288366 | 96 | −45 | gb105pln | Z. mays PGg14 gene for polygalacturonase. | |
| 700382721H1 | g20323 | 53 | −46 | gb105pln | O. sativa RAc1 gene for actin. | |
| 700356236H1 | g2160155 | 14 | 14 | gb105pln | Sequence of BAC F21M12 from Arabidopsis thaliana chromosome 1, complete | |
| | | | | | sequence. | |
| 700353690H1 | g168587 | 67 | −55 | gb105pln | Zea mays cofactor-independent phosphoglycerate mutase mRNA, complete cds. | |
| 700383172H1 | g34234 | 59 | −7 | gb105allp | laminin-binding protein | |
| 700382820H1 | g57702 | 14 | 3 | gb105allp | ribosomal protein L35 (AA 1-123) | |
| 700382711H1 | g414702 | 22 | −3 | gb105pln | H. lanatus mRNA for allergen Hol-lI. | |
| 700356164H1 | g288373 | 54 | −59 | gb105pln | Z. mays PGg6 gene for polygalacturonase. | |
| 700382148H1 | g218157 | 58 | 6 | gb105eukp | cytoplasmic aldolase | |
| 700355956H1 | g311657 | 26 | −12 | gb105pln | P. hybrida flavonol synthase mRNA. | |
| 700382966H1 | g2828278 | 17 | 10 | gb105pln | Arabidopsis thaliana DNA chromosome 4, BAC clone T18B16 (ESSAII project). | |
| 700282445H2 | g1741868 | 25 | −10 | gb105allp | SDF2 | |
| 700382448H1 | g218135 | 23 | −51 | gb105pln | Rice mRNA for heat shock protein 90KD (AK108 gene), partial sequence. | |
| 700357429H1 | g2511530 | 73 | −43 | gb105pn | Eleusine indica alpha tubulin 1 (TUA1) mRNA, complete cds. | |
| 700353986H1 | g2330689 | 15 | 10 | gb105pln | Yeast (Saccaromyces pombe) chromosome I cosmid c16E8. | |
| 700353191H1 | g312083 | 10 | 2 | gb105eukp | NUO-10.5 | |
| 700382015H1 | g1495248 | 25 | −41 | gb105pln | A. thaliana mRNA for unknown protein, eRF1-3 gene. | |
| 700282416H2 | g288366 | 46 | −62 | gb105pln | Z. mays PGg14 gene for polygalacturonase. | |
| 700354405H1 | g431267 | 22 | 11 | gb105pln | Arabidopsis thaliana ubiquitin conjugating enzyme (UBC6) gene, complete cds. | |
| 700355966H1 | g2832707 | 25 | 3 | gb105eukp | T9A21.180; translation initiation factor eIF-2 gamma chain-like protein | |
| 700353576H1 | g540581 | 22 | −3 | gb105eukp | mudrA | |
| 700383072H1 | g2222798 | 6 | 8 | gb105eukp | gibberellin 20-oxidase | |
| 700382424H1 | g2827524 | 24 | 2 | gb105allp | predicted protein | |
| 700352630H1 | g435428 | 33 | −6 | gb105pln | Arabidopsis thaliana GTP-binding protein mRNA. | |
| 700353071H1 | g166379 | 38 | −20 | gb105pln | Alfalfa glucose-regulated endoplasmic reticular protein mRNA, complete cds. | |
| 700357444H1 | g623343 | 23 | 2 | gb105pln | S. oleracea (subito) fad6 mRNA. | |
| 700355560H1 | g1167835 | 25 | −8 | gb105pln | H. lanatus mRNA for Hol lI protein. | |
| 700382374H1 | g2599103 | 20 | 1 | gb105pln | Dunaliella salina 60S ribosomal protein (DSRP1) mRNA, complete cds. | |
| 700357145H1 | g551266 | 25 | −10 | gb105pln | P. argentatum (line 11591) 60S acidic ribosomal protein P2 mRNA. | |
| 700356463H1 | g1906812 | 68 | 1 | gb105allp | inducible carbonyl reductgase | |
| 700354280H1 | g22418 | 73 | −57 | gb105pln | Z. mays gene for polygalacturonase (clone 17). | |
| 700352650H1 | g2182028 | 73 | −62 | gb105pln | Oryza sp. mRNA for shaggy-like kinase etha. | |
| 700352683H1 | g599722 | 22 | −17 | gb105pln | C. melo mRNA for aconitase (UNI-ZAPxR). | |
| 700355972H1 | g306875 | 37 | −24 | gb105allp | C protein | |
| 700352955H1 | g560476 | 78 | −20 | gb105allp | ribosomal protein L4 | |
| 700353162H1 | g22416 | 50 | −58 | gb105pln | Z. mays mRNA for polygalacturonase (clone PG1). | |
| 700382281H1 | g533692 | 11 | 2 | gb105allp | protease inhibitor | |
| 700382777H1 | g453975 | 23 | −30 | gb105pln | P. pratense mRNA for profilin-Phl p XI. | |
| 700354627H1 | g1658192 | 50 | −61 | gb105pln | Sorghum bicolor obtusifoliol 14-alpha demethylase CYP51 (CYP51) mRNA, complete cds. | |
| 700382302H1 | g1532168 | 25 | −4 | gb105eukp | AT.I.24-6 | |
| 700382560H1 | g312180 | 25 | −24 | gb105pln | Z. mays GapC4 gene. | |
| 700357469H1 | g22416 | 48 | −3 | gb105pln | Z. mays mRNA for polygalacturonase (clone PG1). | |
| 700382249H1 | g2814799 | 29 | −9 | gb105eukp | ZK858.6 | |
| 700355207H1 | g22458 | 60 | −85 | gb105pln | Z. mays pollen specific mRNA C-terminal (clone 4H7). | |
| 700382311H1 | g1773190 | 11 | 3 | gb105allp | similar to E. coli yhaE | |
| 700354322H1 | g22425 | 44 | −38 | gb105pln | Z. mays gene for polygalacturonase (clone 47). | |
| 700356039H1 | g22416 | 88 | −79 | gb105pln | Z. mays mRNA for polygalacturonase (clone PG1). | |
| 700352920H1 | g872058 | 26 | 7 | gb105allp | C14A4.4 | |
| 700353129H1 | g1399556 | 14 | −10 | gb105pln | Caltha palustris nuclear 26S ribosomal RNA gene, partial sequence. | |
| 700354947H1 | g531481 | 79 | −17 | gb105pln | P. ciliare (Higgins) apospory associated mRNA, 1398bp. | |
| 700383125H1 | g1302656 | 24 | −5 | gb105allp | translocon-associated protein delta | |
| 700355489H1 | g438253 | 40 | −28 | gb105pln | S. tuberosum mRNA for T subunit of glycine decarboxylase multi-enxyme complex. | |
| 700282577H2 | g1109831 | 40 | −24 | gb105eukp | C16A3.9 | |
| 700356976H1 | g1490417 | 54 | −3 | gb105eukp | mor; global transcriptional regulation; 89B helicase | |
| 700355966H1 | g902603 | 25 | 1 | gb105allp | translation initiation factor | |
| 700382240H1 | g1051108 | 37 | −17 | gb105pln | A. thaliana mRNA for adenine nucleotide translocase. | |
| 700354619H1 | g2583148 | 20 | −6 | gb105eukp | pfk; 6-phospho-1-fructokinase; EC 2.7.1.11 | |
| 700353596H1 | g22458 | 97 | −11 | gb105pln | Z. mays pollen specific mRNA C-terminal (clone 4H7). | |
| 700352736H1 | g2239261 | 38 | −65 | gb105pln | Zea Mays mRNA for pectin methylesterase-like protein. | |
| 700382868H1 | g963095 | 16 | −11 | gb105eukp | 60S ribosomal protein L13A | |
| 700355911H1 | g393449 | 24 | −22 | gb105pln | S. cereale mRNA for beta-amylase. | |
| 700355192H1 | g1906827 | 12 | 14 | gb105pln | A. thaliana hsp81.4 gene. | |
| 700353037H1 | g1143387 | 53 | −22 | gb105pln | A. thaliana mRNA for class III ADH. | |
| 700354360H1 | g1616658 | 64 | −47 | gb105pln | Triticum aestivum adenylosuccinate synthetase mRNA, partial cds. | |
| 700382367H1 | g2351065 | 12 | 12 | gb105pln | Arabidopsis thaliana genomic DNA, chromosome 5, P1 clone: MHF15. | |
| 700356848H1 | g2160158 | 30 | −16 | gb105eukp | F21M12.3 | |
| 700282409H2 | g313137 | 24 | −8 | gb105pln | Z. mays AmPRO1 mRNA for profilin 1. | |
| 700383086H1 | g1881692 | 64 | −70 | gb105pln | Zea mays phosphoglucomutase mRNA, partial cds. | |
| 700357138H1 | g288378 | 76 | −73 | gb105pln | Z. mays gene PG for polygalacturonase. | |
| 700356976H1 | g171965 | 54 | −3 | gb105eukp | MOT1; Mot1 | |
| 700383156H1 | g1246115 | 21 | −2 | gb105pln | major allergen Pha a 1 [Phalaris aquatica = canary grass, pollen, mRNA Partial, | |
| | | | | | 1088 nt]. | |
| 700354705H1 | g2191182 | 8 | 2 | gb105eukp | A_TM021B04.2 | |
| 700383018H1 | g506470 | 19 | 7 | gb105pln | N. tabacum mRNA pNLA-35. | |
| 700353789H1 | g288378 | 31 | −30 | gb105pln | Z. mays gene PG for polygalacturonase. | |
| 700356533H1 | g2290204 | 7 | 6 | gb105eukp | putatively associated with nucellar cell programmed cell death; nucellin; EC 3.4.23 | |
| 700354575H1 | g287829 | 86 | −30 | gb105pln | Z. mays gene for polygalacturonase. | |
| 700382148H1 | g168419 | 62 | 1 | gb105pln | Maize (Z. mays) aldolase mRNA, complete cds. | |
| 700382064H1 | g431164 | 36 | −25 | gb105eukp | LIM17; ORF | |
| 700382628H1 | g166581 | 38 | −39 | gb105pln | A. thaliana AAc1 gene encoding actin-1, complete cds. | |
| 700356084H1 | g288378 | 52 | −59 | gb105pln | Z. mays gene PG for polygalacturonase. | |
| 700355073H1 | g450235 | 48 | 7 | gb105allp | APS kinase | |
| 700383193H1 | g1815758 | 20 | 5 | gb105pln | Phalaris coerulescens pollen-specific protein mRNA, complete cds. | |
| 700382257H1 | g482937 | 42 | −40 | gb105pln | N. tabacum mRNA for pyruvate kinase (plastid isozyme). | |
| 700355594H1 | g1458245 | 14 | −4 | gb105eukp | F54D11.1 | |
| 700353834H1 | g414907 | 16 | 7 | gb105pln | P. pratense mRNA for pollen allergen PhlpI. | |
| 700356050H1 | g2668745 | 100 | −85 | gb105pln | Zea mays inorganic pyrophosphatase (IPP) mRNA, complete cds. | |
| 700353816H1 | g2642215 | 25 | 2 | gb105eukp | NOI protein | |
| 700353152H1 | g1330253 | 43 | 13 | gb105pln | Zea mays calcium-dependent protein kinase mRNA, complete cds. | |
| 700356985H1 | g2244788 | 30 | 9 | gb105pln | Arabidopsis thaliana DNA chromosome 4, ESSA I contig fragment No. 1. | |
| 700353614H1 | g1710150 | 34 | −27 | gb105pln | Arabidopsis thaliana proline iminopeptidase mRNA, complete cds. | |
| 700352784H1 | g576817 | 45 | −20 | gb105eukp | Ape; DNA repair protein | |
| 700354553H1 | g2347209 | 59 | −9 | gb105eukp | T09D09.10; EGF receptor like protein isolog | |
| 700353763H1 | g1877523 | 25 | −5 | gb105pln | Arabidopsis thaliana BAC T7I23, complete sequence. | |
| 700355573H1 | g19850 | 19 | −9 | gb105pln | N. tabacum mRNA for pre-pro-cysteine proteinase. | |
| 700353429H1 | g168608 | 72 | −5 | gb105pln | Maize 17S ribosomal RNA gene and flanks. | |
| 700382237H1 | g556672 | 21 | 2 | gb105pln | S. cereale (Halo) chloroplast mRNA for heat-shock protein. | |
| 700355330H1 | g16112 | 45 | −46 | gb105pln | Avena sativa mRNA for phytochrome (AP4). | |
| 700383057H1 | g984677 | 30 | −11 | gb105pln | P. sativum mRNA for lipoamide dehydrogenase. | |
| 700355075H1 | g2224933 | 20 | −7 | gb105eukp | EIN3; ethylene-insensitive3 | |
| 700382104H1 | g927798 | 14 | −3 | gb105eukp | YDR531W; Ydr531wp | |
| 700355818H1 | g1911581 | 30 | −21 | gb105pln | Cyn d 1 = major allergen {clone 14c1 and CD1} [Cynodon dactylon = Bermuda grass, | |
| | | | | | pollen, mRNA Partial, 759 nt]. | |
| 700382972H1 | g2415701 | 19 | −14 | gb105pln | P. pratense mRNA for profilin 4. | |
| 700382351H1 | g1209258 | 19 | −1 | gb105eukp | protease inhibitior II | |
| 700354724H1 | g971283 | 40 | −36 | gb105pln | Rice mRNA for ribosomal protein S31, complete cds. | |
| 700352816H1 | g22458 | 73 | −30 | gb105pln | Z. mays pollen specific mRNA C-terminal (clone 4H7). | |
| 700282478H2 | g293901 | 35 | −17 | gb105pln | Zea mays Zea mI gene, complete cds. | |
| 700282415H2 | g520596 | 24 | −5 | gb105eukp | MRE2; Mre2 protein | |
| 700282533H2 | g1381677 | 40 | −31 | gb105pln | Glycine max small GTP-binding protein (sra2) mRNA, complete cds. | |
| 700383073H1 | g1498052 | 12 | −1 | gb105pln | Zea mays ribosomal protein S8 mRNA, complete cds. | |
| 700356169H1 | g288378 | 58 | −60 | gb105pln | Z. mays gene PG for polygalacturonase. | |
| 700282438H2 | g1323748 | 12 | 6 | gb105allp | thiol protease | |
| 700357492H1 | g534915 | 33 | −21 | gb105pln | S. tuberosum (Desiree) ppa mRNA for soluble inorganic pyrophosphatase. | |
| 700354271H1 | g288612 | 17 | −0 | gb105eukp | PG; polygalacturonase | |
| 700382715H1 | g13004 | 27 | −0 | gb105allp | URF 1 (NADH dehydrogenase subunit) | |
| 700382607H1 | g16836 | 20 | 2 | gb105allp | probable protease inhibitor P322 precursor (Bowman-Birk) | |
| 700382305H1 | g1103585 | 4 | 8 | gb105allp | laminin beta 2 chain | |
| 700356833H1 | g598075 | 17 | −11 | gb105eukp | KAS III; 3-ketoacyl-acyl carrier protein synthase III (KAS III) | |
| 700354713H1 | g170687 | 27 | −7 | gb105pln | Triticum aestivum DNA binding protein (EmBP-1c) mRNA, partial cds. | |
| 700354248H1 | g414908 | 22 | 8 | gb105eukp | pollen allergen Phl pI | |
| 700382688H1 | g1931579 | 9 | 2 | gb105allp | O-GlcNAc transferase, p110 subunit | |
| 700382002H1 | g600768 | 36 | −36 | gb105pln | Oryza sativa cyclophilin 2 (Cyp2) mRNA, complete cds. | |
| 700352954H1 | g2431770 | 100 | −50 | gb105pln | Zea mays acidic ribosomal protein P2b (rpp2b) mRNA, complete cds. | |
| 700354340H1 | g1067097 | 11 | 3 | gb105eukp | ZK637.8a | |
| 700354250H1 | g854177 | 15 | 2 | gb105allp | RNA polymerase II subunit hRPB17 | |
| 700356950H1 | g1122316 | 52 | −2 | gb105pln | P. glaucum mRNA for heat shock protein, HSP 17.9. | |
| 700356144H1 | g288373 | 34 | −55 | gb105pln | Z. mays PGg6 gene for polygalacturonase. | |
| 700383115H1 | g54006 | 30 | −6 | gb105allp | ribosomal protein S12 (AA 1-132) | |
| 700356990H1 | g22421 | 98 | −38 | gb105pln | Z. mays mRNA for polygalacturonase (clone PG3). | |
| 700355945H1 | g600768 | 50 | −46 | gb105pln | Oryza sativa cyclophilin 2 (Cyp2) mRNA, complete cds. | |
| 700353609H1 | g1668773 | 30 | −1 | gb105eukp | smt3; ubiquitin-like protein | |
| 700357187H1 | g19495 | 29 | −22 | gb105pln | L. perenne RNA for Lol pI gene. | |
| 700354747H1 | g170434 | 20 | −9 | gb105pln | Tomato ATP-dependent protease (CD4B) gene, complete cds. | |
| 700353435H1 | g429010 | 50 | −5 | gb105pln | Rice mRNA for cytochrome c (gene name SS393), partial cds. | |
| 700382392H1 | g2827559 | 9 | 8 | gb105eukp | T12H17.210; predicted protein | |
| 700353633H1 | g1235568 | 23 | −2 | gb105pln | O. sativa mRNA for NAD(P)H oxidase. | |
| 700357449H1 | g1839597 | 65 | −5 | gb105eukp | calcium/calmodulin-dependent protein kinase homolog\|CaM kinase homlog\|MCK1 | |
| 700357492H1 | g2258073 | 46 | −37 | gb105pln | Hordeum vulgare var. distichum soluble inorganic pyrophosphatase (Ipp) mRNA, | |
| | | | | | complete cds. | |
| 700382142H1 | g2293565 | 54 | −36 | gb105pln | Oryza sativa ADP-ribosylation factor 1 (Os-ARF1) mRNA, complete cds. | |
| 700354991H1 | g1217994 | 48 | −1 | gb105eukp | cell plate formation; SDL12A | |
| 700356911H1 | g22421 | 96 | −36 | gb105pln | Z. mays mRNA for polygalacturonase (clone PG3). | |
| 700356445H1 | g1246115 | 20 | 6 | gb105pln | major allergen Pha a 1 [Phalaris aquatica = canary grass, pollen, mRNA Partial, | |
| | | | | | 1088 nt]. | |
| 700353867H1 | g17797 | 15 | −2 | gb105eukp | Bplo | |
| 700352835H1 | g1872162 | 52 | −42 | gb105pln | Arabidopsis thaliana DnaJ homolog (atj) mRNA, complete cds. | |
| 700282447H2 | g577611 | 47 | −36 | gb105pln | Zea mays CRT1 gene for calcium-binding protein. | |
| 700382269H1 | g497239 | 29 | −27 | gb105pln | Zea mays W22 plasma-membrane H+ ATPase (Zmpma1) mRNA, partial cds. | |
| 700282464H1 | g22428 | 12 | 0 | gb105eukp | polygalacturonase | |
| 700356880H1 | g288366 | 98 | −21 | gb105pln | Z. mays PGg14 gene for polygalacturonase. | |
| 700354485H1 | g886400 | 47 | −30 | gb105pln | Oryza sativa MADS-box protein (MADS2) mRNA, complete cds. | |
| 700383194H1 | g168316 | 14 | −6 | gb105eukp | Lol pI; pollen allergen | |
| 700383036H1 | g216535 | 14 | −1 | gb105allp | sensor-regulator protein | |
| 700282486H2 | g1263301 | 29 | −20 | gb105pln | Pisum sativum mitochondrial pyruvate dehydrogenase E1 alpha subunit mRNA, | |
| | | | | | complete cds. | |
| 700354338H1 | g18759 | 11 | 7 | gb105eukp | 28 kD preprotein | |
| 700357164H1 | g533692 | 21 | −3 | gb105eukp | protease inhibitor | |
| 700356479H1 | g939784 | 32 | −4 | gb105pln | Zea mays MADS-box protein (ZAP1) mRNA, complete cds. | |
| 700355014H1 | g1814402 | 44 | −32 | gb105pln | Mesembryanthemum crystallinum methionine synthase (MetE) mRNA, complete cds. | |
| 700383132H1 | g1167836 | 20 | 3 | gb105eukp | hol lI; major group I allergen; protein with incomplete signal sequence | |
| 700383037H1 | g1532047 | 11 | 12 | gb105pln | O. sativa mRNA for S-adenosylmethionine decarboxylase. | |
| 700382344H1 | g1246115 | 23 | −6 | gb105pln | major allergen Pha a 1 [Phalaris aquatica = canary grass, pollen, mRNA Partial, | |
| | | | | | 1088 nt]. | |
| 700356163H1 | g166926 | 9 | −3 | gb105eukp | DNA-damage sensitive. | |
| 700354741H1 | g945023 | 39 | −1 | gb105allp | methylenetetrahydrofolate reductase | |
| 700383038H1 | g395077 | 39 | −19 | gb105eukp | ribosomal protein L37a | |
| 700356511H1 | g22427 | 44 | −65 | gb105pln | Z. mays gene for polygalacturonase (clone 65). | |
| 700382933H1 | g147144 | 5 | 7 | gb105allp | aminopeptidase N | |
| 700382501H1 | g1136119 | 43 | −39 | gb105pln | O. sativa mRNA for alpha-tubulin (clone OSTA-111). | |
| 700355956H1 | g1805308 | 25 | −10 | gb105pln | Arabidopsis thaliana flavonol synthase mRNA, complete cds. | |
| 700355426H1 | g575292 | 9 | 2 | gb105eukp | BKIN2; SNF1-related protein kinase | |
| 700355005H1 | g498774 | 55 | −47 | gb105pln | Z. mays (cv DH5xDH7) hsp70-5 mRNA for heat shock protein 70. | |
| 700382380H1 | g2213594 | 49 | −24 | gb105eukp | T7N9.14 | |
| 700354955H1 | g1217609 | 26 | −5 | gb105allp | Csa-19 homologue | |
| 700353606H1 | g2262169 | 14 | 1 | gb105eukp | F5J6.14; clathrin adaptor protein small chain subunit (AP19 protein) | |
| 700355273H1 | g22458 | 49 | −67 | gb105pln | Z. mays pollen specific mRNA C-termina; (clone 4H7). | |
| 700354408H1 | g2522194 | 40 | −26 | gb105pln | Triticum aestivum ornithine/acetylornithine aminotransferase mRNA, partial cds. | |
| 700352765H1 | g311330 | 9 | 12 | gb105pln | S. vulgare ltp1 gene. | |
| 700382246H1 | g22416 | 65 | −69 | gb105pln | Z. mays mRNA for polygalacturonase (clone PG1). | |
| 700356969H1 | g453975 | 39 | 2 | gb105pln | P. pratense mRNA for profilin-Phl p XI. | |
| 700354459H1 | g2665671 | 37 | −35 | gb105pln | Glycine max proteasome IOTA subunit mRNA, complete cds. | |
| 700352868H1 | g1495251 | 33 | −4 | gb105eukp | heat-shock protein | |
| 700356441H1 | g1825645 | 18 | −2 | gb105eukp | F46F11.4 | |
| 700354663H1 | g804656 | 32 | −2 | gb105eukp | BGO60; beta-glucosidase | |
| 700282491H2 | g470958 | 45 | −17 | gb105pln | Echinodorus cordifolius 18S ribosomal RNA (18S rRNA), ca. bp 294 to 543 in mature | |
| | | | | | rRNA. | |
| 700354330H1 | g1771781 | 22 | −15 | gb105pln | T. aestivum mRNA for beta-amylase. | |
| 700355083H1 | g2181184 | 29 | −13 | gb105eukp | JR3 protein | |
| 700355856H1 | g2330673 | 10 | 5 | gb105eukp | rp122; 60S ribosomal protein | |
| 700355663H1 | g2529386 | 20 | −5 | gb105pln | Zea mays triosephosphate isomerase 1 gene, exon 2-9 and complete cds. | |
| 700355258H1 | g21692 | 49 | 11 | gb105pln | T. aestivum mRNA 1 for cathepsin B (2529). | |
| 700355010H1 | g1556445 | 60 | −33 | gb105pln | Hordeum vulgare alpha tubulin (tubA) mRNA, complete cds. | |
| 700382815H1 | g2160155 | 16 | 10 | gb105pln | Sequence of BAC F21M12 from Arabidopsis thaliana chromosome 1, complete | |
| | | | | | sequence. | |
| 700382493H1 | g414908 | 10 | 1 | gb105allp | pollen allergen Phl pI | |
| 700354638H1 | g2213424 | 24 | −21 | gb105pln | Citrus paradisi mRNA for hypothetical protein. | |
| 700353641H1 | g168490 | 71 | −54 | gb105pln | Maize glutathione S-transferase (GST-I) mRNA, complete cds. | |
| 700355323H1 | g19495 | 23 | 9 | gb105pln | L. perenne RNA for Lol pI gene. | |
| 700353119H1 | g1321660 | 23 | 9 | gb105pln | Rice mRNA for ascorbate peroxidase, complete cds. | |
| 700353064H1 | g313137 | 34 | −75 | gb105pln | Z. mays ZmPRO1 mRNA for profilin 1. | |
| 700354309H1 | g644491 | 96 | −93 | gb105pln | Corn mRNA for elongation factor 1A. | |
| 700353855H1 | g2213586 | 7 | 8 | gb105eukp | T7N9.6 | |
| 700382711H1 | g414907 | 22 | −3 | gb105pln | P. pratense mRNA for pollen allergen PhlpI. | |
| 700352919H1 | g20163 | 40 | −7 | gb105pln | O. sativa Rr15 mRNA for 5S ribosomal RNA. | |
| 700382510H1 | g22423 | 63 | −63 | gb105pln | Z. mays mRNA for polygalacturonase (clone PG4). | |
| 700356575H1 | g313141 | 27 | −67 | gb105pln | Z. mays ZmPRO3 mRNA for profilin 3. | |
| 700353135H1 | g1654002 | 17 | −1 | gb105allp | Sop2p-like protein | |
| 700356248H1 | g899607 | 61 | −27 | gb105pln | Zea mays polyubiquitin (MubC5) mRNA, complete cds. | |
| 700352793H1 | g1707639 | 72 | −55 | gb105pln | O. sativa mRNA for transcription activator, GAMyb. | |
| 700382892H1 | g1184773 | 39 | −56 | gb105pln | Zea mays glyceraldehyde-3-phosphate dehydrogenase GAPC3 (gpc3) mRNA, | |
| | | | | | complete cds. | |
| 700356130H1 | g1911765 | 9 | 1 | gb105eukp | iEP4; iEP4 | |
| 700382389H1 | g2827142 | 36 | −29 | gb105pln | Arabidopsis thaliana cellulose synthase catalytic subunit (Ath-B) mRNA, complete cds. | |
| 700357440H1 | g2351070 | 12 | 17 | gb105pln | Arabidopsis thaliana genomic DNA, chromosome 5, P1 clone: MTH12. | |
| 700382866H1 | g1143392 | 32 | −13 | gb105eukp | uridine diphosphate glucose epimerase; EC 5.1.3.2 | |
| 700354274H1 | g2547025 | 36 | −16 | gb105allp | XY40 protein | |
| 700382181H1 | g868002 | 32 | −28 | gb105pln | Pumpkin mRNA for aconitase, complete cds. | |
| 700356137H1 | g1707867 | 59 | −74 | gb105pln | Z. mays mRNA for 40S ribosomal subunit protein S21. | |
| 700357428H1 | g20310 | 15 | 2 | gb105eukp | pollen specific gene | |
| 700353258H1 | g414907 | 25 | −9 | gb105pln | P. pratense mRNA for pollen allergen PhlpI. | |
| 700354657H1 | g20255 | 55 | −44 | gb105pln | O. sativa gene for heat shock protein 82 HSP82. | |
| 700353723H1 | g169705 | 45 | 6 | gb105eukp | ATP:pyruvate phosphotransferase; EC 2.7.1.40; PK-p-beta | |
| 700352693H1 | g2244993 | 17 | 4 | gb105eukp | similarity to AMP-activated protein kinase beta | |
| 700355969H1 | g1419684 | 33 | −17 | gb105pln | M. sativa mRNA for TCTP-like protein. | |
| 700355754H1 | g1246115 | 18 | −26 | gb105pln | major allergen Pha a 1 [Phalaris aquatica = canary grass, pollen, mRNA Partial, | |
| | | | | | 1088 nt]. | |
| 700352809H1 | g2580498 | 21 | −10 | gb105pln | Eschscholtzia californica NADPH:ferrihemoprotein oxidoreductase mRNA, | |
| | | | | | complete cds. | |
| 700382608H1 | g22416 | 96 | −89 | gb105pln | Z. mays mRNA for polygalacturonase (clone PG1). | |
| 700357187H1 | g1167835 | 32 | −23 | gb105pln | H. lanatus mRNA for Hol lI protein. | |
| 700352659H1 | g1655678 | 21 | −0 | gb105pln | P. sylvestris mRNA for 3-hydroxy-3-methylglutaryl-CoA synthase. | |
| 700357430H1 | g1673354 | 24 | 6 | gb105allp | salt-stress induced hydrophobic peptide | |
| 700355492H1 | g313137 | 49 | −29 | gb105pln | Z. mays ZmPRO1 mRNA for profilin 1. | |
| 700355460H1 | g2673869 | 22 | −0 | gb105pln | Antirrhinum majus mRNA for fimbriata-associated protein 2, partial. | |
| 700354410H1 | g1143501 | 31 | −29 | gb105pln | H. vulgare mRNA for ADP-glucose pyrophosphorylase small subunit (1902 bp). | |
| 700354542H1 | g2331300 | 33 | −48 | gb105pln | Zea mays ribosomal protein S4 type I (rps4) mRNA, complete cds. | |
| 700354852H1 | g313026 | 19 | 4 | gb105pln | L. esculentum rp138 mRNA for ribosomal protein L38. | |
| 700382824H1 | g248336 | 66 | −73 | gb105pln | polyubiquitin [maize, Genomic, 3841 nt]. | |
| 700355992H1 | g1825645 | 42 | −8 | gb105eukp | F46F11.4 | |
| 700382737H1 | g453975 | 40 | −27 | gb105pln | P. pratense mRNA for profilin-Phl p XI. | |
| 700356273H1 | g2052366 | 62 | 5 | gb105allp | protein 5 | |
| 700353794H1 | g453975 | 24 | −1 | gb105pln | P. pratense mRNA for profilin-Phl p XI. | |
| 700352890H1 | g2645970 | 37 | −42 | gb105pln | Arabidopsis thaliana reversivly glycosylated polypeptide-3 (RGP) mRNA, complete cds. | |
| 700355188H1 | g2274990 | 69 | −65 | gb105pln | Hordeum vulgare mRNA for expressed sequence tag. | |
| 700353639H1 | g22427 | 82 | −74 | gb105pln | Z. mays gene for polygalacturonase (clone 65). | |
| 700353635H1 | g393706 | 56 | −20 | gb105pln | C. sativus mRNA for 3-ketoacyl-CoA thiolase. | |
| 700354902H1 | g442525 | 33 | 5 | gb105pln | ZmERabp4 = auxin-binding protein [Zea mays = corn, seedling, mRNA, 917 nt]. | |
| 700382966H1 | g2828279 | 35 | −9 | gb105eukp | T18B16.10; hypothetical protein | |
| 700355701H1 | g288373 | 41 | −11 | gb105pln | Z. mays PGg6 gene for polygalacturonase. | |
| 700354841H1 | g21800 | 26 | −31 | gb105pln | T. aestivum L mRNA for histone H2B. | |
| 700355783H1 | g2351063 | 19 | 7 | gb105pln | Arabidopsis thaliana genomic DNA, chromosome 5, P1 clone: MCL19. | |
| 700353802H1 | g2529661 | 15 | −2 | gb105eukp | T30B22.4; putative dTDP-glucose 4-6-dehydratase | |
| 700353418H1 | g22418 | 94 | −42 | gb105pln | Z. mays gene for polygalacturonase (clone 17). | |
| 700354227H1 | g414703 | 30 | −1 | gb105eukp | allergen Hol-lI | |
| 700382882H1 | g1657852 | 16 | 10 | gb105pln | Triticum aestivum cold acclimation protein WCOR825 (Wcor825) mRNA, partial cds. | |
| 700356494H1 | g2852444 | 19 | 14 | gb105pln | Salix bakko mRNA for SUI1 homolog, complete cds. | |
| 700353591H1 | g2760171 | 26 | −1 | gb105pln | Arabidopsis thaliana genomic DNA, chromosome 5, P1 clone: MPA24, complete | |
| | | | | | sequence. | |
| 700383094H1 | g971283 | 47 | −33 | gb105pln | Rice mRNA for ribosomal protein S31, complete cds. | |
| 700352886H1 | g17797 | 14 | 1 | gb105eukp | Bplo | |
| 700353237H1 | g929917 | 20 | 5 | gb105pln | Z. mays ABP1 mRNA for actin-depolymerizing factor. | |
| 700355905H1 | g2342735 | 25 | 5 | gb105eukp | T14G11.28 | |
| 700355226H1 | g414908 | 11 | 4 | gb105eukp | pollen alergen Phl pI | |
| 700354920H1 | g288611 | 98 | −61 | gb105pln | Z. mays PG gene for polygalacturonase. | |
| 700354833H1 | g22272 | 31 | −57 | gb105pln | Maize mRNA for enolase (2-phospho-D-glycerate hydrolase). | |
| 700353218H1 | g2189964 | 19 | −20 | gb105eukp | Phosphoglycerate dehydrogenase | |
| 700353074H1 | g286121 | 63 | −64 | gb105pln | Maize mRNA for glutamine synthetase, complete cds. | |
| 700352794H1 | g2656031 | 15 | 3 | gb105pln | Arabidopsis thaliana genomic DNA, chromosome 5, P1 clone: MXC20. | |
| 700356745H1 | g393449 | 28 | 1 | gb105pln | S. cereale mRNA for beta-amylase. | |
| 700352754H1 | g1707856 | 63 | −48 | gb105pln | T. aestivum mRNA for obtusifoliol 14-alpha-demethylase (CYP51), clone w516, partial. | |
| 700353107H1 | g456567 | 33 | −12 | gb105pln | Pisum sativum ubiquitin conjugating enzyme (UBC4), complete cds. | |
| 700352708H1 | g2293006 | 10 | −2 | gb105allp | hypothetical protein | |
| 700282585H2 | g414702 | 21 | −3 | gb105pln | H. lanatus mRNA for allergen Hol-lI. | |
| 700352990H1 | g170772 | 79 | −14 | gb105pln | Triticum aestivum S-adenosyl-L-homocysteine hydrolase (SH6.2) mRNA, complete cds. | |
| 700357413H1 | g1777929 | 74 | −68 | gb105pln | Saccharum officinarum nucleoside diphosphate kinase (SoNDPK1) mRNA, | |
| | | | | | complete cds. | |
| 700353016H1 | g829147 | 94 | −18 | gb105pln | Z. mays gene for cyclophilin. | |
| 700382129H1 | g1617270 | 14 | −3 | gb105eukp | acyl-CoA synthetase; EC 6.2.1.3 | |
| 700382013H1 | g1644320 | 15 | 2 | gb105eukp | SPAC6G9.08; putative ubiquitin carboxyl terminal hydrolase | |
| 700357171H1 | g2832242 | 20 | 1 | gb105pln | Zea mays 22-kDa alpha zein gene cluster, complete sequence. | |
| 700382608H1 | g22421 | 96 | −89 | gb105pln | Z. mays mRNA for polygalacturonase (clone PG3). | |
| 700357468H1 | g297797 | 59 | 0 | gb105pln | S. tuberosum mRNA for formate dehydrogenase. | |
| 700352687H1 | g1208445 | 52 | −39 | gb105pln | Rice (YK426) mRNA, complete cds. | |
| 700352977H1 | g633135 | 29 | 4 | gb105pln | Zostera marina mRNA for putative plasma membrane H+-ATPase, complete cds. | |
| 700382249H1 | g2435604 | 32 | −9 | gb105eukp | F08F1.7 | |
| 700354333H1 | g2352262 | 36 | −24 | gb105eukp | peroxidoxin-1 | |
| 700356834H1 | g22418 | 81 | −80 | gb105pln | Z. mays gene for polygalacturonase (clone 17). | |
| 700355659H1 | g288366 | 21 | −11 | gb105pln | Z. mays PGg14 gene for polygalacturonase. | |
| 700352804H1 | g2687724 | 19 | −5 | gb105eukp | NADPH:isoflavone reductase | |
| 700382609H1 | g311906 | 34 | −20 | gb105pln | N. tabacum mRNA for ras-related GTP-binding protein. | |
| 700355293H1 | g20321 | 42 | −25 | gb105pln | Oryza sativa RAc1 mRNA for actin. | |
| 700282527H2 | g168315 | 20 | 9 | gb105pln | L. perenne pollen allergen (Lol pI) gene, complete cds. | |
| 700355576H1 | g577611 | 64 | −68 | gb105pln | Zea mays CRT1 gene for calcium-binding protein. | |
| 700355934H1 | g21794 | 75 | −58 | gb105pln | Wheat histone H4 gene. | |
| 700356492H1 | g2266661 | 51 | −26 | gb105pln | Hordeum vulgare mRNA for 14-3-3 protein (Hv1433c). | |
| 700356555H1 | g2564252 | 33 | 10 | gb105pln | Hordeum vulgare mRNA for diadenosine 5′, 5′′′-P1,P4-tetraphosphate hydrolase. | |
| 700382385H1 | g2286150 | 92 | −78 | gb105pln | Zea mays translation initiaition factor (eIF-4A) mRNA, complete cds. | |
| 700382105H1 | g414702 | 24 | −7 | gb105pln | H. lanatus mRNA for allergen Hol-lI. | |
| 700382636H1 | g2584827 | 10 | 16 | gb105pln | Sequence of BAC F12F1 from Arabidopsis thaliana chromosome 1, complete sequence. | |
| 700357439H1 | g432488 | 35 | −3 | gb105pln | Wheat initiation factor 1A (eIF-1A) mRNA. | |
| 700355634H1 | g169776 | 17 | −4 | gb105pln | Oryza sativa beta-amylase gene, complete cds. | |
| 700354583H1 | g703066 | 72 | −11 | gb105eukp | ste13+; RNA helicase | |
| 700382474H1 | g1185555 | 11 | 16 | gb105pln | Zea mays glyceraldehyde-3-phosphate dehydrogenase (gpc4) gene, partial cds. | |
| 700354224H1 | g22427 | 63 | 5 | gb105pln | Z. mays gene for polygalacturonase (clone 65). | |
| 700353810H1 | g168569 | 49 | −12 | gb105pln | Zea mays putative phenylalanine ammonia lyase mRNA, partial cds. | |
| 700353168H1 | g1184775 | 38 | −38 | gb105pln | Zea mays glyceraldehyde-3-phosphate dehydrogenase GAPC4 (gpc4) mRNA, | |
| | | | | | complete cds. | |
| 700356010H1 | g536053 | 10 | 8 | gb105eukp | URA7 | |
| 700355756H1 | g1532047 | 30 | −40 | gb105pln | O. sativa mRNA for S-adenosylmethionine decarboxylase. | |
| 700355326H1 | g22458 | 60 | −78 | gb105pln | Z. mays pollen specific mRNA C-terminal (clone 4H7). | |
| 700353641H1 | g168486 | 67 | −64 | gb105pln | Maize glutathione S-transferase gene (GST-I), exon 1. | |
| 700282408H2 | g313137 | 23 | −1 | gb105pln | Z. mays ZmPRO1 mRNA for profilin 1. | |
| 700382209H1 | g2384759 | 39 | −17 | gb105pln | Oryza sativa GDP dissociation inhibitor protein OsGDI2 (OsGDI2) mRNA, | |
| | | | | | complete cds. | |
| 700353256H1 | g1732519 | 40 | −16 | gb105eukp | Rho1Ps homolog | |
| 700382102H1 | g556672 | 15 | 8 | gb105pln | S. cereale (Halo) chloroplast mRNA for heat-shock protein. | |
| 700356137H1 | g1419371 | 55 | −38 | gb105pln | Z. mays mRNA for 40S subunit ribosomal protein S21. | |
| 700354308H1 | g22458 | 67 | −83 | gb105pln | Z. mays pollen specific mRNA C-terminal (clone 4H7). | |
| 700353087H1 | g2760164 | 17 | 11 | gb105pln | Arabidopsis thaliana genomic DNA, chromosome 5, TAC clone: K18P6, complete sequence. | |
| 700354866H1 | g288373 | 31 | −36 | gb105pln | Z. mays PGg6 gene for polygalacturonase. | |
| 700352772H1 | g22302 | 36 | −64 | gb105pln | Maize Gpc1 gene for glyceraldehyde-3-phosphate dehydrogenase (GADPH) subunit C. | |
| 700355556H1 | g2702284 | 6 | 3 | gb105eukp | T21L14.12; Argonaute (AGO1)-like protein | |
| 700382777H1 | g313139 | 25 | −31 | gb105pln | Z. mays ZmPRO2 mRNA for profilin 2. | |
| 700382380H1 | g2182287 | 20 | 1 | gb105pln | Genomic sequence for Arabidopsis thaliana BAC T7N9, complete sequence. | |
| 700355226H1 | g1173557 | 13 | 4 | gb105eukp | Ory s 1 | |
| 700353523H1 | g22427 | 46 | −42 | gb105pln | Z. mays gene for polygalacturonase (clone 65). | |
| 700353261H1 | g288611 | 81 | −70 | gb105pln | Z. mays PG gene for polygalacturonase. | |
| 700382391H1 | g602252 | 98 | −79 | gb105pln | Zea mays enolase (eno2) mRNA, complete cds. | |
| 700382005H1 | g22155 | 62 | −74 | gb105pln | Z. mays mRNA for alpha-tubulin 5. | |
| 700354441H1 | g807063 | 31 | −19 | gb105allp | immunoglobulin JC kappa chain [human, B cell precursor acute lymphocytic leukemia | |
| | | | | | cell line pre-ALP, Peptide, 135 aa] | |
| 700355169H1 | g2341032 | 9 | 3 | gb105eukp | F19P19.11 | |
| 700356416H1 | g2288887 | 30 | −17 | gb105eukp | MVD1; mevalonate diphosphate decarboxylase; EC 4.1.1.33 | |
| 700356094H1 | g388928 | 12 | −3 | gb105pln | Zea mays calcium dependent protein kinase (CDPK) mRNA, partial cds. | |
| 700356288H1 | g777757 | 24 | −26 | gb105pln | Saccharum hybrid (clone SCUBI561) polyubiquitin mRNA, complete cds. | |
| 700355935H1 | g248338 | 63 | −64 | gb105pln | polyubiquitin [maize, Genomic 3439 nt]. | |
| 700355867H1 | g1658192 | 76 | −71 | gb105pln | Sorghum bicolor obtusifoliol 14-alpha demethylase CYP51 (CYP51) mRNA, | |
| | | | | | complete cds. | |
| 700354248H1 | g473360 | 22 | 8 | gb105eukp | phl pI; Phl pI allergen | |
| 700353631H1 | g1335861 | 21 | −4 | gb105pln | Glycine max clathrin heavy chain mRNA, complete cds. | |
| 700382637H1 | g166548 | 25 | −23 | gb105pln | Avena sativa vacuolar H+-ATPase 16 kDa proteolipid subunit (vatp-P1) mRNA, complete cds. | |
| 700382267H1 | g2058693 | 10 | 7 | gb105allp | poly A binding protein | |
| 700355693H1 | g1438881 | 19 | 2 | gb105eukp | GmCK2p; EC 2.7.1.32 | |
| 700353952H1 | g1049090 | 14 | 5 | gb105allp | SRp40-2 | |
| 700352614H1 | g1048989 | 37 | −5 | gb105allp | CD9 antigen | |
| 700282422H2 | g414907 | 18 | 10 | gb105pln | P. pratense mRNA for pollen allergen PhlpI. | |
| 700383195H1 | g557598 | 14 | 11 | gb105pln | Yeast (Saccharomyces cerevisiae) ATPase gene, complete cds. | |
| 700382133H1 | g2689469 | 20 | 5 | gb105eukp | IAA22 | |
| 700355949H1 | g1498052 | 40 | −31 | gb105pln | Zea mays ribosomal protein S8 mRNA, complete cds. | |
| 700354609H1 | g414907 | 41 | −40 | gb105pln | P. pratense mRNA for pollen allergen PhlpI. | |
| 700356789H1 | g1041424 | 65 | −19 | gb105pln | H. vulgare mRNA for glutamyl-tRNA reductase, 1st isoform. | |
| 700356565H1 | g17795 | 14 | 1 | gb105eukp | bplo | |
| 700353038H1 | g1864023 | 74 | −24 | gb105pln | Cynodon dactylon calcium-binding pollen allergen gene, partial cds. | |
| 700282415H2 | g2708532 | 24 | −8 | gb105eukp | QRRBP-1; putative RNA binding protein | |
| 700353834H1 | g414908 | 8 | 8 | gb105eukp | pollen allergen Phl pI | |
| 700352748H1 | g289189 | 34 | −19 | gb105pln | Arabidopsis thaliana calcium dependent protein kinase (AK1 isoform) mRNA, | |
| | | | | | complete cds. | |
| 700354878H1 | g1262910 | 14 | 1 | gb105eukp | cytidine triphosphate synthetase; EC 6.3.4.2 | |
| 700383194H1 | g473360 | 15 | −6 | gb105eukp | phl pI; Phl pI allergen | |
| 700356817H1 | g2160177 | 22 | −1 | gb105eukp | F21M12.26 | |
| 700353255H1 | g1345546 | 6 | 2 | gb105eukp | Npg1; polygalacturonase; EC 3.2.1.15 | |
| 700355543H1 | g1246102 | 26 | −6 | gb105allp | CAG-isl 7 [human, pancreas, Peptide Partial, 213 aa] | |
| 700354726H1 | g493709 | 40 | −21 | gb105pln | Rice mRNA for beta-tubulin, complete cds. | |
| 700382427H1 | g780261 | 30 | 7 | gb105allp | lactate dehydrogenase-A | |
| 700355209H1 | g2674203 | 58 | −10 | gb105eukp | CLPX; CLP protease regulatory subunit CLPX | |
| 700382237H1 | g556673 | 24 | −1 | gb105eukp | heat-shock protein | |
| 700355243H1 | g397958 | 98 | −25 | gb105pln | Z. mays mRNA for beta-amylase. | |
| 700383193H1 | g20309 | 14 | 14 | gb105pln | O. sativa DNA for pollen-specific gene. | |
| 700355948H1 | g1246841 | 14 | −3 | gb105eukp | g2827 | |
| 700382868H1 | g1743007 | 16 | −9 | gb105eukp | ribosomal protein L13a | |
| 700282422H2 | g168315 | 19 | 9 | gb105pln | L. perenne pollen allergen (Lol pI) gene, complete cds. | |
| 700356013H1 | g22416 | 37 | −79 | gb105pln | Z. mays mRNA for polygalacturonase (clone PG1). | |
| 700355844H1 | g2108252 | 8 | 1 | gb105eukp | pgp2; P-glycoprotein-2 | |
| 700352676H1 | g166548 | 47 | −29 | gb105pln | Avena sativa vacuolar H+-ATPase 16 kDa proteolipid subunit (vatp-P1) mRNA, | |
| | | | | | complete cds. | |
| 700354342H1 | g19538 | 34 | −31 | gb105pln | M. crystallinum mRNA for ribosomal protein YL16. | |
| 700356747H1 | g1388021 | 39 | 1 | gb105allp | UDP-glucose pyrophosphorylase | |
| 700355824H1 | g2801669 | 15 | −1 | gb105pln | Mycosphaerella graminicola succinate dehydrogenase iron-sulphur protein gene, | |
| | | | | | complete cds. | |
| 700353234H1 | g1881379 | 22 | −4 | gb105pln | Solanum melongena mRNA for ribosomal protein L5, partial cds. | |
| 700353135H1 | g1749452 | 11 | 7 | gb105eukp | similar to Saccharomyces cerevisiae hypothetical 42.5KD protein in PDB1-ABD1 | |
| | | | | | intergenic region, SWISS-PROT Accession Number P38328 | |
| 700353107H1 | g2668743 | 68 | −46 | gb105pln | Zea mays ubiquitin conjugating enzyme (UBC) mRNA, complete cds. | |
| 700355026H1 | g2707295 | 6 | 7 | gb105eukp | root allergen protein | |
| 700353268H1 | g20309 | 19 | −8 | gb105pln | O. sativa DNA for pollen-specific gene. | |
| 700356073H1 | g288378 | 41 | −61 | gb105pln | Z. mays gene PG for polygalacturonase. | |
| 700353611H1 | g16440 | 30 | −14 | gb105pln | A. thaliana PRKase gene for Ribulose-5-phosphate kinase. | |
| 700356814H1 | g293901 | 65 | −45 | gb105pln | Zea mays Zea mI gene, complete cds. | |
| 700352631H1 | g1532210 | 42 | −0 | gb105eukp | cypA; cyclophilin A | |
| 700355522H1 | g22456 | 57 | −77 | gb105pln | Z. mays pollen specific mRNA C-terminal (clone 3C12). | |
| 700353237H1 | g1419367 | 22 | 2 | gb105pln | Z. mays ZmABP2 mRNA for actin depolymerizing factor. | |
| 700353268H1 | g1815758 | 15 | 5 | gb105pln | Phalaris coerulescens pollen-specific protein mRNA, complete cds. | |
| 700355982H1 | g218130 | 30 | −25 | gb105pln | Rice mRNA for Ribosomal protein S15. | |
| 700353528H1 | g288366 | 88 | −82 | gb105pln | Z. mays PGg14 gene for polygalacturonase. | |
| 700357140H1 | g22066 | 21 | −7 | gb105eukp | sulfhydryl-endopeptidase | |
| 700354891H1 | g2827524 | 39 | −8 | gb105eukp | F8F16.110; predicted protein | |
| 700356225H1 | g22464 | 32 | −68 | gb105pln | Maize rbcS gene for ribulose-1,5-bisphosphate carboxylase/oxygenase small subunit | |
| | | | | | (EC 4.1.1.39). | |
| 700354920H1 | g288378 | 76 | −46 | gb105pln | Z. mays gene PG for polygalacturonase. | |
| 700355193H1 | g1749733 | 32 | −21 | gb105pln | Fission Yeast mRNA, partial cds. | |
| 700382507H1 | g2351063 | 23 | 16 | gb105pln | Arabidopsis thaliana genomic DNA, chromosome 5, P1 clone: MCL19. | |
| 700356279H1 | g1762930 | 28 | −8 | gb105pln | Nicotiana tabacum ribosomal protein S14 mRNA, partial cds. | |
| 700353148H1 | g1556375 | 17 | 15 | gb105pln | A. niger gene ANNUO51 for 51 kDa subunit of NADH:ubiquinone reductase (complex 1). | |
| 700382787H1 | g22651 | 50 | −36 | gb105pln | H. vulgare mRNA for GRP94 homologue. | |
| 700355740H1 | g473359 | 24 | −7 | gb105pln | P. pratense mRNA for Phl pI allergen. | |
| 700382027H1 | g1402874 | 41 | −34 | gb105pln | A. thaliana 81kb genomic sequence. | |
| 700357431H1 | g2668747 | 92 | −80 | gb105pln | Zea mays ribosomal protein L17 (rp117) mRNA, complete cds. | |
| 700354944H1 | g1184773 | 81 | −47 | gb105pln | Zea mays glyceraldehyde-3-phosphate dehydrogenase GAPC3 (gpc3) mRNA, | |
| | | | | | complete cds. | |
| 700355401H1 | g31062 | 7 | 5 | gb105allp | Epstein-Barr virus small RNA associated protein | |
| 700382655H1 | g1718238 | 10 | 1 | gb105allp | (1,4)-beta-xylan endohydrolase, isoenzyme X-II | |
| 700382393H1 | g1132482 | 33 | −4 | gb105pln | Rice mRNA for ADP-ribosylation factor, complete cds. | |
| 700352940H1 | g533692 | 10 | 5 | gb105allp | protease inhibitor | |
| 700354471H1 | g641999 | 13 | −12 | gb105eukp | cytochrome oxidase I | |
| 700382789H1 | g1103629 | 30 | −76 | gb105pln | Z. mays Fer2 gene. | |
| 700382617H1 | g22458 | 48 | −87 | gb105pln | Z. mays pollen specific mRNA C-terminal (clone 4H7). | |
| 700355815H1 | g1145627 | 17 | −10 | gb105eukp | lipase | |
| 700353171H1 | g397958 | 67 | −23 | gb105pln | Z. mays mRNA for beta-amylase. | |
| 700382636H1 | g2781363 | 22 | −2 | gb105eukp | F24O1.19 | |
| 700352779H1 | g2341032 | 31 | −10 | gb105eukp | F19P19.11 | |
| 700354905H1 | g2282583 | 55 | −64 | gb105pln | Zea mays elongation factor 1-alpha (EF10-A) mRNA, complete cds. | |
| 700354690H1 | g22421 | 63 | −49 | gb105pln | Z. mays mRNA for polygalacturonase (clone PG3). | |
| 700353272H1 | g2160169 | 17 | −4 | gb105eukp | F21M12.17 | |
| 700354543H1 | g1556428 | 44 | −7 | gb105pln | N. tabacum mRNA for citrate synthase. | |
| 700353294H1 | g1015849 | 12 | −2 | gb105eukp | RPS5 | |
| 700382206H1 | g1655423 | 42 | −24 | gb105pln | Arabidopsis thaliana mRNA for GDP dissociation inhibitor, complete cds. | |
| 700352672H1 | g22651 | 69 | −57 | gb105pln | H. vulgare mRNA for GRP94 homologue. | |
| 700382493H1 | g473360 | 10 | 1 | gb105allp | Phl pI allergen | |
| 700382376H1 | g1064931 | 29 | −5 | gb105eukp | cyclin A-like protein | |
| 700353256H1 | g1293668 | 40 | −19 | gb105eukp | ARAC5; GTP-binding protein | |
| 700355563H1 | g473360 | 20 | −3 | gb105eukp | phl pI; Phl pI allergen | |
| 700355169H1 | g2244792 | 11 | 1 | gb105eukp | ankyrin homolog | |
| 700356048H1 | g1167835 | 23 | −1 | gb105pln | H. lanatus mRNA for Hol lI protein. | |
| 700354917H1 | g287829 | 98 | −62 | gb105pln | Z. mays gene for polygalacturonase. | |
| 700352755H1 | g168460 | 61 | −58 | gb105pln | Zea mays cyclophilin (CyP) mRNA, complete cds. | |
| 700352972H1 | g20310 | 22 | 4 | gb105eukp | pollen specific gene | |
| 700354816H1 | g2642157 | 8 | 6 | gb105eukp | T5I7.5; ankyrin-like protein | |
| 700356660H1 | g531031 | 69 | −24 | gb105pln | Pennisetum ciliare apomixis-associated mRNA. | |
| 700356258H1 | g1575129 | 92 | −89 | gb105pln | Zea mays lumenal binding protein cBiPe3 mRNA, complete cds. | |
| 700353136H1 | g1518259 | 21 | 1 | gb105allp | gpi8 | |
| 700352660H1 | g473359 | 25 | −7 | gb105pln | P. pratense mRNA for Phl pI allergen. | |
| 700354820H1 | g1914844 | 61 | −73 | gb105pln | Zea mays liguleless1 protein (liguleless1) mRNA, complete cds. | |
| 700382956H1 | g2570506 | 31 | −12 | gb105pln | Oryza sativa ribosomal protein mRNA, complete cds. | |
| 700382849H1 | g499011 | 56 | −61 | gb105pln | S. vulgare SoAc1 mRNA. | |
| 700382119H1 | g473877 | 11 | 15 | gb105pln | Arabidopsis thaliana Columbia calnexin homolog gene, complete cds. | |
| 700382048H1 | g22416 | 43 | −68 | gb105pln | Z. mays mRNA for polygalacturonase (clone PG1). | |
| 700356777H1 | g960356 | 61 | −20 | gb105pln | Barley mRNA for S-adenosylmethionine synthetase, complete cds. | |
| 700352968H1 | g22223 | 17 | −12 | gb105pln | Maize cab-1 gene for chlorophyll a/b-binding protein. | |
| 700353222H1 | g290275 | 33 | −12 | gb105eukp | M(3)95A; multifunctional activity and location; ribosomal protein S3/AP endonclease | |
| | | | | | DNA repair protein | |
| 700353261H1 | g288378 | 61 | −49 | gb105pln | Z. mays gene PG for polygalacturonase. | |
| 700356933H1 | g1402878 | 23 | 6 | gb105allp | unknown | |
| 700355812H1 | g288366 | 73 | −85 | gb105pln | Z. mays PGg14 gene for polygalacturonase. | |
| 700353962H1 | g1184773 | 65 | −22 | gb105pln | Zea mays glyceraldehyde-3-phosphate dehydrogenase (gpc3) mRNA, complete cds. | |
| 700382946H1 | g20321 | 37 | −29 | gb105pln | Oryza sativa RAc1 mRNA for actin. | |
| 700382779H1 | g19701 | 42 | −10 | gb105pln | N. plumbaginifolia mRNA NeIF-5A2 for initiation factor 5A(2). | |
| 700382611H1 | g473359 | 24 | −7 | gb105pln | P. pratense mRNA for Phl pI allergen. | |
| 700282411H2 | g414702 | 26 | −10 | gb105pln | H. lanatus mRNA for allergen Hol-lI. | |
| 700282575H2 | g2829898 | 11 | 4 | gb105eukp | T26J12.9 | |
| 700353981H1 | g22151 | 37 | −3 | gb105pln | Z. mays (A188) mRNA for alpha-tubulin 4. | |
| 700353267H1 | g836945 | 15 | −9 | gb105pln | Arabidopsis thaliana calcium-dependent protein kinase (CDPK9) gene, complete cds. | |
| 700353286H1 | g2642323 | 25 | −35 | gb105pln | Zea mays profilin (PRO4) mRNA, complete cds. | |
| 700354615H1 | g2766714 | 22 | −0 | gb105pln | Arabidopsis arenosa isolate Care-1 (9509) chromomethylase (CMT1) mRNA, partial cds. | |
| 700357444H1 | g457630 | 42 | −18 | gb105pln | Brassica napus proplastid omega-6 desaturase mRNA, complete cds. | |
| 700355649H1 | g2058278 | 10 | 4 | gb105eukp | atran1 | |
| 700352989H1 | g287829 | 72 | −55 | gb105pln | Z. mays gene for polygalacturonase. | |
| 700354145H1 | g1732362 | 11 | 17 | gb105pln | Malus domestica pAFD103 mRNA, partial cds. | |
| 700354575H1 | g288366 | 85 | −30 | gb105pln | Z. mays PGg14 gene for polygalacturonase. | |
| 700356564H1 | g499011 | 46 | −53 | gb105pln | S. vulgare SoAc1 mRNA. | |
| 700353081H1 | g2852447 | 17 | 6 | gb105eukp | APK2a; protein kinase | |
| 700282544H2 | g633135 | 37 | −13 | gb105pln | Zostera marina mRNA for putative plasma membrane H+-ATPase, complete cds. | |
| 700354227H1 | g414908 | 31 | 0 | gb105eukp | pollen allergen Phl pI | |
| 700355437H1 | g886428 | 22 | −9 | gb105eukp | P1; putative NADPH oxidoreductase; zeta-crystallin homlogue | |
| 700353064H1 | g453975 | 26 | −29 | gb105pln | P. pratense mRNA for profilin-Phl pXI. | |
| 700355839H1 | g1125690 | 18 | −71 | gb105pln | S. tuberosum mRNA for DnaJ protein. | |
| 700352933H1 | g288378 | 100 | −22 | gb105pln | Z. mays gene PG for polygalacturonase. | |
| 700355696H1 | g22428 | 11 | 8 | gb105eukp | polygalacturonase | |
| 700355770H1 | g2213424 | 36 | −23 | gb105pln | Citrus paradisi mRNA for hypothetical protein. | |
| 700356170H1 | g1246115 | 19 | 0 | gb105pln | major allergen Pha a1 [Phalaris aquatica = canary grass, pollen, mRNA Partial, | |
| | | | | | 1088 nt]. | |
| 700353841H1 | g19500 | 44 | −31 | gb105pln | Lupinus polyphyllus lupin-specific pPLZ12 mRNA. | |
| 700382662H1 | g1815758 | 19 | 4 | gb105pln | Phalaris coerulescens pollen-specific protein mRNA, complete cds. | |
| 700356858H1 | g804656 | 11 | −7 | gb105eukp | BGQ60; beta-glucosidase | |
| 700382412H1 | g22425 | 92 | −16 | gb105pln | Z. mays gene for polygalacturonase (clone 47). | |
| 700382902H1 | g1649012 | 9 | 0 | gb105eukp | egg receptor for sperm | |
| 700353536H1 | g414908 | 32 | 7 | gb105eukp | pollen allergen Phl pI | |
| 700354309H1 | g644492 | 96 | −92 | gb105pln | Corn elongation factor 1alpha gene, complete cds. | |
| 700353543H1 | g1632821 | 57 | −34 | gb105pln | O. sativa mRNA for transmembrane protein. | |
| 700354611H1 | g607003 | 7 | 7 | gb105allp | beta transducin-like protein | |
| 700353917H1 | g22425 | 64 | 1 | gb105pln | Z. mays gene for polygalacturonase (clone 47). | |
| 700354566H1 | g435428 | 15 | −7 | gb105pln | Arabidopsis thaliana GTP-binding protein mRNA. | |
| 700383036H1 | g1789149 | 14 | −1 | gb105allp | sensor protein BarA | |
| 700354287H1 | g2811229 | 32 | −15 | gb105pln | Arabidopsis thaliana fimbrin 1 gene, partial cds. | |
| 700382932H1 | g2113935 | 24 | 1 | gb105allp | unknown | |
| 700382319H1 | g22427 | 76 | −76 | gb105pln | Z. mays gene for polygalacturonase (clone 65). | |
| 700352636H1 | g1255959 | 6 | 8 | gb105allp | ORF N0840 | |
| 700282554H2 | g2463586 | 10 | 4 | gb105eukp | precursor of cysteine proteinase; FB22 precursor | |
| 700353245H1 | g1815758 | 23 | 1 | gb105pln | Phalaris coerulescens pollen-specific protein mRNA, complete cds. | |
| 700353696H1 | g2809231 | 21 | 11 | gb105pln | Genomic sequence for Arabidopsis thaliana BAC F22O13, complete sequence. | |
| 700354817H1 | g2832242 | 30 | −14 | gb105pln | Zea mays 22-kDa alpha zein gene cluster, complete sequence. | |
| 700382883H1 | g1504051 | 19 | −14 | gb105pln | Maize; corn mRNA for Calcium-dependent protein kinase, complete cds. | |
| 700382920H1 | g22416 | 28 | −58 | gb105pln | Z. mays mRNA for polygalacturonase (clone Pg1). | |
| 700383140H1 | g2245073 | 26 | −20 | gb105pln | Arabidopsis thaliana DNA chromosome 4, ESSA I contig fragment No. 8. | |
| 700356933H1 | g2252840 | 18 | 7 | gb105allp | contains regions of similarity to Haemophilua influenzae permease (SP:P38767) | |
| 700352602H1 | g202529 | 9 | 0 | gb105allp | 3-alpha-hydroxysteroid dehydrogenase | |
| 700382448H1 | g170455 | 34 | −48 | gb105pln | Tomato heat shock cognate protein 80 gene, 3′ end. | |
| 700355492H1 | g453975 | 43 | −24 | gb105pln | P. pratense mRNA for profilin-Phl pXI. | |
| 700354974H1 | g2342682 | 28 | −0 | gb105eukp | F7G19.11 | |
| 700353954H1 | g297015 | 27 | −8 | gb105eukp | fructokinase | |
| 700356082H1 | g609261 | 16 | 1 | gb105pln | S. cereale (cv. Halo) mRNA for triosephosphate isomerase. | |
| 700355958H1 | g499011 | 82 | −70 | gb105pln | S. vulgare SoAc1 mRNA. | |
| 700353943H1 | g587565 | 19 | 9 | gb105pln | S. tuberosum mRNA for beta-I MPP. | |
| 700353253H1 | g2264311 | 12 | 7 | gb105pln | Arabidopsis thaliana genomic DNA chromosome 5, P1 clone: MLN1, complete | |
| | | | | | sequence. | |
| 700354642H1 | g2342737 | 15 | 2 | gb105eukp | T14G11.30; Ser/Thr protein kinase isolog, 5′ partial | |
| 700357428H1 | g1815759 | 16 | 1 | gb105eukp | pollen-specific protein | |
| 700383014H1 | g473359 | 21 | −5 | gb105pln | P. pratense mRNA for Phl pI allergen. | |
| 700383076H1 | g1167835 | 18 | 3 | gb105pln | H. lanatus mRNA for Hol lI protein. | |
| 700356435H1 | g2852444 | 20 | 12 | gb105pln | Salix bakko mRNA for SUI1 homolog, complete cds. | |
| 700355974H1 | g747848 | 21 | −3 | gb105pln | H. vulgare (Gerbel) mRNA for adenosine triphosphatase. | |
| 700355001H1 | g1532162 | 16 | 17 | gb105pln | arabidopsis thaliana AT.I.24-1, AT.I.24-2, AT.I.24-3, AT.I.24-4, AT.I.24-5, AT.I.24-6, | |
| | | | | | AT.I.24-9 and AT.I.24-14 genes, partial cds, AT.I.24-7, ascorbate peroxidase | |
| | | | | | (ATHAPX1), EF-1alpha-A1, -A2 and -A3 (EF-1alpha) and AT.I.24-13 genes, | |
| | | | | | complete cds. | |
| 700282404H2 | g683778 | 4 | 8 | gb105eukp | unknown | |
| 700352705H1 | g1030068 | 17 | −4 | gb105eukp | NAD(P)H oxidoreductase, isoflavone reductase homologue | |
| 700382903H1 | g1458245 | 11 | 4 | gb105eukp | F54D11.1 | |
| 700356463H1 | g1906814 | 58 | 3 | gb105allp | non-inducible carbony reductase | |
| 700382850H1 | g1658321 | 26 | −9 | gb105pln | S. tuberosum mRNA for transketolase. | |
| 700282470H2 | g2511596 | 12 | 6 | gb105eukp | prce; multicatalytic endopeptidase complex, proteasome precursor, beta subunit; | |
| | | | | | EC 3.4.99.46 | |
| 700382304H1 | g777757 | 33 | −48 | gb105pln | Saccharum hybrid (clone SCUBI561) polyubiquitin mRNA, complete cds. | |
| 700382268H1 | g2213586 | 5 | 5 | gb105eukp | T7N9.6 | |
| 700356229H1 | g1905943 | 23 | 2 | gb105pln | Sorghum bicolor MADS box transcription factor SbMADS1 mRNA, complete cds. | |
| 700352779H1 | g2244792 | 30 | −8 | gb105eukp | ankyrin homolog | |
| 700383114H1 | g287829 | 84 | −69 | gb105pln | Z. mays gene for polygalacturonase. | |
| 700356847H1 | g288611 | 39 | 13 | gb105pln | Z. mays PG gene for polygalacturonase. | |
| 700356574H1 | g288373 | 68 | −76 | gb105pln | Z. mays PGg6 gene for polygalacturonase. | |
| 700356494H1 | g20237 | 17 | 9 | gb105pln | O. sativa (rice) constitutive GOS2 gene. | |
| 700356057H1 | g1806384 | 26 | 1 | gb105allp | by similarity to bovine sequence | |
| 700357410H1 | g2104525 | 16 | −9 | gb105eukp | T10M13.2; T10M13.2 | |
| 700382737H1 | g313139 | 44 | −30 | gb105pln | Z. mays ZmPRO2 mRNA for profilin 2. | |
| 700382282H1 | g1209258 | 17 | −1 | gb105eukp | protease inhibitor II | |
| 700353794H1 | g313139 | 48 | −57 | gb105pln | Z. mays ZmPRO2 mRNA for profilin 2. | |
| 700282523H2 | g1246116 | 16 | 7 | gb105eukp | major allergen Pha a 1; major allergen Pha a 1 | |
| 700382171H1 | g643596 | 70 | −72 | gb105pln | Corn mRNA for cysteine proteinase, clone CCP, complete cds. | |
| 700355015H1 | g20255 | 40 | −20 | gb105pln | O. sativa gene for heat shock protein 82 HSP82. | |
| 700352939H1 | g1020409 | 12 | 5 | gb105eukp | phospholipase D; EC 3.1.4.4 | |
| 700356248H1 | g777757 | 58 | −19 | gb105pln | Saccharum hybrid (clone SCUBI561) polyubiquitin mRNA, complete cds. | |
| 700353547H1 | g1439635 | 14 | 1 | gb105eukp | F56B6.4 | |
| 700357419H1 | g1785739 | 16 | −5 | gb105eukp | tRNA-Ser | |
| 700354250H1 | g1213454 | 10 | 5 | gb105allp | similar to S. cerevisiae DNA-directed RNA polymerases I, II and II (SP:RPB8_YEAST, | |
| | | | | | P20436) | |
| 700355977H1 | g285637 | 53 | −41 | gb105pln | Hordeum vulgare mRNA for vacuolar membrane proton-translocating inorganic | |
| | | | | | pyrophosphat. | |
| 700355337H1 | g248336 | 69 | −70 | gb105pln | polyubiquitin [maize, Genomic, 3841 nt]. | |
| 700282493H2 | g19902 | 48 | 7 | gb105eukp | pollen specific protein | |
| 700353067H1 | g2218151 | 33 | −10 | gb105pln | Vigna unguiculata type IIIa membrane protein cp-wap13 mRNA, complete cds. | |
| 700355935H1 | g902583 | 69 | −69 | gb105pln | Zea mays clone MubG1 ubiquitin gene, complete cds. | |
| 700382057H1 | g2829925 | 27 | −12 | gb105eukp | F22K20.12 | |
| 700356254H1 | g1246213 | 7 | 6 | gb105allp | AICAR transformylase-IMP cyclohydrolase | |
| 700356095H1 | g304986 | 9 | 8 | gb105allp | similar to 3-hydroxyisobutyrate dehydrogenases; similar to E. coli hypoth. 31 kDa protein | |
| | | | | | in rnpB 3′ region | |
| 700383071H1 | g2529721 | 11 | −1 | gb105allp | unknown | |
| 700353991H1 | g393449 | 27 | −24 | gb105pln | S. cereale mRNA for beta-amylase. | |
| 700356555H1 | g2564253 | 43 | 5 | gb105allp | diadenosine 5′, 5′′′-P1, P4-tetraphosphate hydrolase | |
| 700282482H2 | g1788562 | 8 | 6 | gb105allp | DNA gyrase subunit A | |
| 700356917H1 | g1167827 | 58 | −17 | gb105pln | C. reinhardtii mRNA for ribosomal protein L12. | |
| 700356208H1 | g313137 | 96 | −38 | gb105pln | Z. mays ZmPRO1 mRNA for profilin 1. | |
| 700383086H1 | g1881694 | 57 | −64 | gb105pln | Zea mays phosphoglucomutase mRNA, partial cds. | |
| 700382373H1 | g1403043 | 13 | 8 | gb105pln | H. chilense x T. turgidum conv. durum (Tritordeum) mRNA for S-adenosylmethionine | |
| | | | | | decarboxylase. | |
| 700353891H1 | g1817577 | 30 | −22 | gb105allp | alpha-globin 1 | |
| 700382440H1 | g1197168 | 53 | −27 | gb105eukp | MHCemb; embryonic muscle myosin heavy chain | |
| 700352942H1 | g237649 | 35 | −16 | gb105pln | enoyl-acyl carrier protein reductase [Brassica napus, mRNA, 1358 nt]. | |
| 700355817H1 | g1218042 | 37 | −17 | gb105eukp | PAP1; secreted purple acid phosphatase precursor | |
| 700357468H1 | g297798 | 95 | −6 | gb105eukp | mitochondrial formate dehydrogenase precursor; EC 1.2.1.2 | |
| 700356123H1 | g473359 | 26 | −11 | gb105pln | P. pratense mRNA for Phl pI allergen. | |
| 700356789H1 | g1039331 | 65 | −19 | gb105pln | H. vulgare mRNA for glutamyl-tRNA reductase (1st isoform). | |
| 700354712H1 | g217998 | 12 | 15 | gb105pln | Potato tuber, alpha-glucan phosphorylase (EC 2.4.1.1), type L isozyme gene, | |
| | | | | | complete cds. | |
| 700353902H1 | g804656 | 30 | −9 | gb105eukp | BGQ60; beta-glucosidase | |
| 700356002H1 | g2104438 | 13 | −0 | gb105eukp | SPAC57A7.03c; unknown | |
| 700353641H1 | g22314 | 78 | −78 | gb105pln | Maize mRNA for GSH gluthathione S-transferase I (GST; EC 2.5.1.18). | |
| 700354622H1 | g2244913 | 27 | 8 | gb105eukp | similar to SEN1 protein - yeast; Author-given protein sequence is in conflict with the | |
| | | | | | conceptual translation | |
| 700355460H1 | g1432082 | 20 | 2 | gb105pln | Arabidopsis thaliana Skplp homolog mRNA, complete cds. | |
| 700353816H1 | g2642217 | 21 | 3 | gb105eukp | NOI protein | |
| 700353888H1 | g172320 | 19 | −2 | gb105eukp | RAD16; excision repair protein | |
| 700354434H1 | g2282583 | 36 | −35 | gb105pln | Zea mays elongation factor a-alpha (EF1-A) mRNA, complete cds. | |
| 700355002H1 | g2662344 | 59 | −36 | gb105pln | Oryza sativa mRNA for EF-1 alpha, complete cds. | |
| 700356048H1 | g414702 | 21 | −0 | gb105pln | H. lanatus mRNA for allergen Hol-lI. | |
| 700355562H1 | g736271 | 70 | −64 | gb105pln | O. sativa hsp70 gene for heat shock protein 70. | |
| 700355075H1 | g2224935 | 20 | −7 | gb105eukp | EIN3; ethylene-insensitive3 | |
| 700353796H1 | g2244779 | 24 | 1 | gb105eukp | hypothetical protein | |
| 700355229H1 | g288373 | 73 | −70 | gb105pln | Z. mays PGg6 gene for polygalacturonase. | |
| 700354943H1 | g887594 | 15 | 6 | gb105eukp | unknown | |
| 700352766H1 | g16323 | 19 | −7 | gb105pln | A. thaliana H3 gene 1 and H3 gene 2 for H3.3-like histone variant. | |
| 700357479H1 | g2623679 | 59 | −21 | gb105pln | Zea mays calmodulin (Zmrcalm) mRNA, complete cds. | |
| 700355243H1 | g1771781 | 79 | −17 | gb105pln | T. aestivum mRNA for beta-amylase. | |
| 700354955H1 | g416514 | 28 | −7 | gb105allp | NEDD-6 | |
| 700282568H2 | g1498628 | 27 | −13 | gb105pln | Arabidopsis thaliana p40 protein homolog mRNA, complete cds. | |
| 700382818H1 | g166745 | 38 | −32 | gb105pln | A. thaliana plasma membrane proton ATPase (PMA) mRNA, complete cds. | |
| 700355934H1 | g168500 | 77 | −58 | gb105pln | Maize (Zea mays) histone H4 gene (H4C14), complete cds. | |
| 700355830H1 | g2723470 | 21 | −1 | gb105pln | Oryza sativa mRNA for sucrose transporter, complete cds. | |
| 700356666H1 | g20359 | 62 | −27 | gb105pln | Rice gene for 17S ribosomal RNA. | |
| 700355953H1 | g39209 | 46 | −37 | gb105pln | S. polyrrhiza mRNA for D-myo-inositol-3-phosphate synthase. | |
| 700353642H1 | g2244747 | 16 | −5 | gb105pln | Arabidopsis thaliana DNA chromosome 4, ESSA I contig fragment No. 0. | |
| 700353627H1 | g2290382 | 28 | −18 | gb105eukp | ppt-1; serine/threonine protein phosphatase PPT1 | |
| 700356240H1 | g2342563 | 37 | −24 | gb105pln | Chlamydomonas reinhardtii Csbp gene. | |
| 700354231H1 | g1184773 | 96 | −48 | gb105pln | Zea mays glyceraldehyde-3-phosphate dehydrogenase GAPC3 (gpc3) mRNA, | |
| | | | | | complete cds. | |
| 700382541H1 | g556642 | 38 | −14 | gb105allp | Nascent polypeptide associated complex alpha subunit | |
| 700382669H1 | g511080 | 42 | −33 | gb105pln | A. thaliana HEM15 mRNA for ferrochelatase. | |
| 700382584H1 | g2583133 | 48 | −1 | gb105eukp | F4L23.29 | |
| 700355952H1 | g414702 | 31 | −11 | gb105pln | H. lanatus mRNA for allergen Hol-lI. | |
| 700353255H1 | g1345547 | 6 | 2 | gb105eukp | NPG; polygalacturonase; EC 3.2.1.15 | |
| 700354771H1 | g2224915 | 7 | 2 | gb105eukp | beta-expansin | |
| 700382105H1 | g1246115 | 24 | −7 | gb105pln | major allergen Pha a 1 [Phalaris aquatica = canary grass, pollen, mRNA Partial, | |
| | | | | | 1088 nt]. | |
| 700357444H1 | g459961 | 46 | −21 | gb105pln | Glycine max proplastid omega-6 desaturase mRNA, complete cds. | |
| 700353708H1 | g288611 | 23 | −19 | gb105pln | Z. mays PG gene for polygalacturonase. | |
| 700352605H1 | g939932 | 10 | 6 | gb105allp | allergen Lol pII | |
| 700353539H1 | g2829274 | 23 | 15 | gb105pln | Arabidopsis thaliana nucleoside diphosphate kinase 3 (ndpk3) mRNA, complete cds. | |
| 700356282H1 | g414704 | 19 | −18 | gb105pln | O. sativa mRNA for cytochrome b5. | |
| 700354748H1 | g804944 | 30 | −6 | gb105eukp | wpk4; wpk4 protein kinase | |
| 700382165H1 | g168423 | 94 | −82 | gb105pln | Zea mays polypeptide chain-binding protein mRNA, 3′ end. | |
| 700355583H1 | g169835 | 24 | −7 | gb105pln | Secale cereale RKIN1 mRNA, complete CDS. | |
| 700352829H1 | g2462925 | 20 | 4 | gb105allp | GTP cyclohydrolase II/3,4-dihydroxy-2-butanone-4-phosphate synthase | |
| 700352887H1 | g285637 | 66 | −16 | gb105pln | Hordeum vulgare mRNA for vacuolar membrane proton-translocating inorganic | |
| | | | | | pyrophosphat. | |
| 700355327H1 | g2464911 | 6 | 4 | gb105eukp | splicing factor homolog | |
| 700353695H1 | g1246115 | 18 | 1 | gb105pln | major allergen Pha a 1 [Phalaris aquatica = canary grass, pollen, mRNA | |
| | | | | | Partial, 1088 nt]. | |
| 700356494H1 | g2668739 | 55 | −47 | gb105pln | Zea mays translation initiation factor GOS2 (TIF) mRNA, complete cds. | |
| 700357423H1 | g288611 | 66 | −76 | gb105pln | Z. mays PG gene for polygalacturonase. | |
| 700356576H1 | g974837 | 34 | 4 | gb105allp | phosphotyrosyl phosphatase activator (PTPA) | |
| 700352764H1 | g168460 | 81 | −68 | gb105pln | Zea mays cyclophilin (CyP) mRNA, complete cds. | |
| 700282469H2 | g22427 | 63 | −89 | gb105pln | Z. mays gene for polygalacturonase (clone 65). | |
| 700354390H1 | g22458 | 95 | −88 | gb105pln | Z. mays pollen specific mRNA C-terminal (clone 4H7). | |
| 700282411H2 | g414907 | 26 | −10 | gb105pln | P. pratense mRNA for pollen allergen PhlpI. | |
| 700354906H1 | g21394 | 27 | −2 | gb105eukp | put. preprotein (AA -27 to 47) | |
| 700353153H1 | g533473 | 33 | −23 | gb105pln | Mesembryanthemum crystallinum 2-phospho-D-glycerate hydrolase, enolase, mRNA, | |
| | | | | | complete cds. | |
| 700355842H1 | g1945365 | 9 | 3 | gb105allp | copper transport protein HAH1 | |
| 700355153H1 | g20280 | 24 | −36 | gb105pln | Rice gene for phenylalanine ammonia-lyase (EC 4.3.1.5). | |
| 700382111H1 | g1885343 | 56 | −60 | gb105pln | T. aestivum mRNA for starch branching enzyme II. | |
| 700354322H1 | g22427 | 44 | −38 | gb105pln | Z. mays gene for polygalacturonase (clone 65). | |
| 700282504H2 | g603415 | 17 | 8 | gb105eukp | YER174C; Yer174cp | |
| 700352929H1 | g2370092 | 52 | 1 | gb105allp | lysosomal hyaluronidase | |
| 700382374H1 | g20163 | 43 | −29 | gb105pln | O. sativa Rr15 mRNA for 5S ribosomal RNA. | |
| 700352641H1 | g1206012 | 77 | −41 | gb105pln | Zea mays beta-D-glucosidase precursor (glu2) mRNA, complete cds. | |
| 700354562H1 | g218082 | 23 | 9 | gb105pln | Rice mRNA for initiation factor eIF-4D (225 gene), partial sequence. | |
| 700382117H1 | g2618677 | 11 | 6 | gb105pln | Arabidopsis thaliana BAC F21B7 chromosome 1, complete sequence. | |
| 700382493H1 | g1167836 | 10 | 1 | gb105eukp | hol lI; major group I allergen; protein with incomplete signal sequence | |
| 700352703H1 | g2262117 | 16 | 6 | gb105eukp | T19F06.20; auxin inducible protein isolog | |
| 700356606H1 | g1871506 | 48 | −13 | gb105pln | c. dactylon mRNA for calcium-binding pollen allergen. | |
| 700354501H1 | g22464 | 61 | −21 | gb105pln | Maize rbcS gene for ribulose-1,5-bisphosphate carboxylase/oxygenase small subunit | |
| | | | | | (EC 4.1.1.39). | |
| 700356256H1 | g899607 | 71 | −82 | gb105pln | Zea mays polyubiquitin (MubC5) mRNA, complete cds. | |
| 700382265H1 | g1049252 | 87 | −57 | gb105pln | Zea mays vacuolar ATPase 69 kDa subunit mRNA, partial cds. | |
| 700354725H1 | g2058455 | 52 | −44 | gb105pln | Arabidopsis thaliana GTP-binding protein (ATDRG1) mRNA, complete cds. | |
| 700356990H1 | g22418 | 98 | −38 | gb105pln | Z. mays gene for polygalacturonase (clone 17). | |
| 700355563H1 | g1167836 | 19 | −2 | gb105eukp | hol lI; major group I allergen; protein with incomplete signal sequence | |
| 700382389H1 | g1706955 | 31 | −24 | gb105pln | Gossypium hirsutum cellulose synthase (celA1) mRNA, complete cds. | |
| 700354640H1 | g2340104 | 13 | 5 | gb105allp | Very similar and perhaps identical to Hs-CUL-4B.; 80-100% similarity to partial | |
| | | | | | sequence U58091 (PID:g1381150). | |
| 700382226H1 | g2702272 | 29 | −7 | gb105eukp | T21L14.13 | |
| 700353610H1 | g2852444 | 15 | −3 | gb105pln | Salix bakko mRNA for SUI1 homolog, complete cds. | |
| 700353233H1 | g22425 | 54 | −52 | gb105pln | Z. mays for polygalacturonase (clone 47). | |
| 700354741H1 | g2301335 | 39 | −1 | gb105allp | unnamed protein product | |
| 700382707H1 | g6433479 | 20 | −5 | gb105eukp | GCN20; Gcn20p | |
| 700352841H1 | g1478229 | 14 | 5 | gb105allp | unknown | |
| 700282558H2 | g2213632 | 8 | 2 | gb105eukp | F21J9.24 | |
| 700353680H1 | g312180 | 30 | −49 | gb105pln | Z. mays GapC4 gene. | |
| 700354894H1 | g288373 | 60 | −67 | gb105pln | Z. mays PGp6 gene for polygalacturonase. | |
| 700352839H1 | g1314098 | 30 | −2 | gb105eukp | YME1; Yme1p | |
| 700353658H1 | g1929411 | 53 | −48 | gb105pln | N. tabacum npp5 mRNA for protein phosphatase type 2A. | |
| 700356013H1 | g288378 | 37 | −79 | gb105pln | Z. mays gene PG for polygalacturonase. | |
| 700354227H1 | g473360 | 31 | −0 | gb105eukp | phl pI; Phl pI allergen | |
| 700355803H1 | g299925 | 22 | −9 | gb105eukp | Dpse\Rp49; ribosomal protein 49 | |
| 700382758H1 | g1200238 | 59 | −54 | gb105pln | H. vulgare mRNA for GAMyb protein. | |
| 700353180H1 | g927571 | 69 | −73 | gb105pln | Z. mays mRNA for calreticulin precursor. | |
| 700355803H1 | g1519359 | 22 | −9 | gb105eukp | RP49; ribosomal protein 49 | |
| 700352809H1 | g1359895 | 19 | −7 | gb105pln | H. tuberosus mRNA for NADPH-ferrihemoprotein reductase. | |
| 700356050H1 | g534915 | 25 | −1 | gb105pln | S. tuberosum (Desiree) ppa mRNA for soluble inorganic pyrophosphatase. | |
| 700353854H1 | g1167835 | 42 | −27 | gb105pln | H. lanatus mRNA for Hol lI protein. | |
| 700357493H1 | g17797 | 22 | 7 | gb105eukp | Bplo | |
| 700353127H1 | g2827625 | 19 | −1 | gb105eukp | putative protein | |
| 700356279H1 | g2994 | 16 | −16 | gb105pln | Neurospora crassa crp-2 gene for ribosomal protein crp-2. | |
| 700382892H1 | g1184775 | 36 | −47 | gb105pln | Zea mays glyceraldehyde-3-phosphate dehydrogenase GAPC4 (gpc4) mRNA, | |
| | | | | | complete cds. | |
| 700356911H1 | g22418 | 96 | −36 | gb105pln | Z. mays gene for polygalacturonase (clone 17). | |
| 700353536H1 | g473360 | 32 | 7 | gb105eukp | phl pI; Phl pI allergen | |
| 700354485H1 | g886402 | 49 | −29 | gb105pln | Oryza sativa MADS-box protein (MADS4) mRNA, complete cds. | |
| 700352808H1 | g1045304 | 48 | −64 | gb105pln | Zea mays acetyl-coenzyme A carboxylase mRNA, complete cds. | |
| 700355809H1 | g2511540 | 51 | −39 | gb105pln | Oryza sativa DNA-binding protein GBP16 (Rgbp16) mRNA, complete cds. | |
| 700355163H1 | g288373 | 94 | −44 | gb105pln | Z. mays PGg6 gene for polygalacturonase. | |
| 700352755H1 | g829147 | 61 | −72 | gb105pln | Z. mays gene for cyclophilin. | |
| 700382823H1 | g1184773 | 59 | −63 | gb105pln | Zea mays glyceraldehyde-3-phosphate dehydrogenase GAPC3 (gpc3) mRNA, | |
| | | | | | complete cds. | |
| 700352988H1 | g287829 | 98 | −55 | gb105pln | Z. mays gene for polygalacturonase. | |
| 700383111H1 | g1627596 | 6 | 8 | gb105allp | C25A1.3 | |
| 700355627H1 | g902583 | 18 | −5 | gb105pln | Zea mays clone MubG1 ubiquitin gene, complete cds. | |
| 700354316H1 | g1040729 | 6 | 6 | gb105allp | cytochrome b5 containing fusion protein | |
| 700354583H1 | g157594 | 66 | −10 | gb105eukp | RNA helicase | |
| 700356685H1 | g288366 | 93 | −37 | gb105pln | Z. mays PGg14 gene for polygalacturonase. | |
| 700355995H1 | g1616660 | 77 | −65 | gb105pln | Zea mays adenylosuccinate synthetase mRNA, complete cds. | |
| 700353002H1 | g2245066 | 21 | 0 | gb105eukp | Beta-Amylase | |
| 700353511H1 | g28549 | 45 | −37 | gb105allp | alpha globin | |
| 700352763H1 | g313141 | 54 | −48 | gb105pln | Z. mays ZmPRO3 mRNA for profilin 3. | |
| 700354379H1 | g22237 | 65 | −56 | gb105pln | Maize mRNA for cytosolic GAPDH (GapC) glyceraldehyde-3-phosphate dehydrogenase. | |
| 700354705H1 | g2344896 | 10 | −1 | gb105eukp | T13E15.11 | |
| 700282416H2 | g288373 | 46 | −61 | gb105pln | Z. mays PGg6 gene for polygalacturonase. | |
| 700355809H1 | g1657616 | 31 | −14 | gb105pln | Arabidopsis thaliana putative nuclear DNA-binding protein G2p (AtG2) mRNA, | |
| | | | | | complete cds. | |
| 700352953H1 | g606814 | 35 | −6 | gb105pln | Zea mays Golden Bantam carbonic anhydrase mRNA, complete cds. | |
| 700353246H1 | g2431768 | 29 | −34 | gb105pln | Zea mays acidic ribosomal protein P1a (rpp1a) mRNA, complete cds. | |
| 700383135H1 | g2392895 | 35 | −16 | gb105eukp | BRI1; brassinosteroid insensitive 1 | |
| 700357129H1 | g1246115 | 29 | −20 | gb105pln | major allergen Pha a 1 [Phalaris aquatica = canary grass, pollen, mRNA Partial, | |
| | | | | | 1088 nt]. | |
| 700355594H1 | g1706965 | 7 | 4 | gb105eukp | TA-MT; delta-24-sterol methyltransferase | |
| 700382907H1 | g16427 | 19 | 2 | gb105eukp | protease inhibitor II | |
| 700382655H1 | g1813595 | 11 | 0 | gb105allp | xylan endohydrolase isoenzyme X-I | |
| 700355634H1 | g397958 | 21 | −14 | gb105pln | Z. mays mRNA for beta-amylase. | |
| 700353601H1 | g17645 | 28 | 6 | gb105allp | RIBOSOMAL PROTEIN L35a | |
| 700355973H1 | g1277236 | 12 | 2 | gb105eukp | SEC13; Sec13p | |
| 700352852H1 | g22418 | 89 | −75 | gb105pln | Z. mays gene for polygalacturonase (clone 17). | |
| 700353708H1 | g288378 | 22 | −18 | gb105pln | Z. mays gene PG for polygalacturonase. | |
| 700357178H1 | g1246115 | 29 | −19 | gb105pln | major allergen Pha a 1 [Phalaris aquatica = canary grass, pollen, mRNA Partial, | |
| | | | | | 1088 nt]. | |
| 700356025H1 | g1657854 | 18 | 8 | gb105pln | Triticum aestivum cold acclimation protein WCOR413 (Wcor413) mRNA, complete cds. | |
| 700356236H1 | g2160158 | 27 | −4 | gb105eukp | F21M12.3 | |
| 700355192H1 | g348695 | 32 | −34 | gb105pln | catharanthus roseus heat shock protein 90 mRNA, complete cds. | |
| 700356130H1 | g1009720 | 8 | 2 | gb105eukp | ENOD8; nodulin | |
| 700382242H1 | g871507 | 14 | 12 | gb105pln | P. sativum mRNA for small GTP-binding protein (clone pGTP13). | |
| 700353037H1 | g1841501 | 95 | −49 | gb105pln | Z. mays mRNA for glutathione-dependent formaldehyde dehydrogenase. | |
| 700353120H1 | g1229168 | 35 | −22 | gb105pln | Hordeum vulgare profilin (Hypro1) mRNA, complete cds. | |
| 700356288H1 | g1839582 | 62 | −42 | gb105pln | polyubiquitin homolog {clone CHEM 6} [Zea mays = maize, cv. INRA 258, mercuric | |
| | | | | | chloride-treated, leaves, mRNA Partial, 199 nt, segment 1 of 2]. | |
| 700352874H1 | g1542941 | 27 | −5 | gb105eukp | AACT; Acetoacetyl-coenzyme A thiolase; EC 2.3.1.9 | |
| 700355543H1 | g1430944 | 28 | −2 | gb105allp | ribosomal protein L14 | |
| 700355765H1 | g20204 | 83 | 4 | gb105eukp | frustose-diphosphate aldolase (AA 1-358) | |
| 700353064H1 | g313139 | 28 | −31 | gb105pln | Z. mays ZmPRO2 mRNA for profilin 2. | |
| 700354479H1 | g836941 | 19 | −4 | gb105pln | Arabidopsis thaliana calcium-dependent protein kinase (CDPK19) mRNA, complete cds. | |
| 700357153H1 | g312180 | 27 | −81 | gb105pln | Z. mays GapC4 gene. | |
| 700382770H1 | g2245020 | 28 | −3 | gb105eukp | growth regulator homolog | |
| 700355921H1 | g2414636 | 7 | 8 | gb105eukp | SPAC3A11.08; hypothetical protein | |
| 700383048H1 | g467617 | 18 | −5 | gb105eukp | minor allergen, ribosomal protein | |
| 700383004H1 | g1574093 | 8 | 4 | gb105allp | histidinol-phosphate aminotransferase (hisH) | |
| 700354383H1 | g1550721 | 15 | −2 | gb105eukp | HPR1; RNA binding protein | |
| 700355750H1 | g2258073 | 14 | 16 | gb105pln | Hordeum vulgare var. distichum soluble inorganic pyrophosphatase (Ipp) mRNA, | |
| | | | | | complete cds. | |
| 700382317H1 | g2689242 | 21 | 3 | gb105pln | A. thaliana mRNA for ubiquitin conjugating enzyme UBC2. | |
| 700382649H1 | g469232 | 7 | 7 | gb105allp | vacuolar H+-ATPase subunit | |
| 700354759H1 | g16427 | 17 | 5 | gb105eukp | protease inhibitor II | |
| 700353113H1 | g287829 | 97 | −80 | gb105pln | Z. mays gene for polygalacturonase. | |
| 700355030H1 | g429013 | 32 | −21 | gb105pln | Rice mRNA for pyrophosphate-fructose 6-phosphate 1-phosphotransferase (gene name | |
| | | | | | SS420), partial cds. | |
| 700382439H1 | g455419 | 12 | 6 | gb105allp | PR264/SC35 | |
| 700356127H1 | g310575 | 36 | −22 | gb105pln | Glycine max nodulin-26 mRNA, complete cds. | |
| 700353262H1 | g2370231 | 20 | −35 | gb105pln | Hordeum vulgare mRNA for putative acyl-CoA oxidase. | |
| 700353525H1 | g287829 | 51 | −59 | gb105pln | Z. mays gene for polygalacturonase. | |
| 700354870H1 | g1086860 | 14 | 1 | gb105eukp | T03G11.4 | |
| 700355985H1 | g1519250 | 49 | −37 | gb105pln | Oryza sativa F14-c protein mRNA, complete cds. | |
| 700282455H2 | g288373 | 53 | −79 | gb105pln | Z. mays PGg6 gene for polygalacturonase. | |
| 700382657H1 | g1419367 | 37 | −14 | gb105pln | Z. mays ZmABP2 mRNA for actin depolymerizing factor. | |
| 700356089H1 | g1216483 | 32 | −18 | gb105pln | Arabidopsis thaliana dual specificity kinase 1 (ADK1) mRNA, complete cds. | |
| 700355948H1 | g1167801 | 31 | −16 | gb105eukp | F38E11.5 | |
| 700353129H1 | g1399559 | 14 | −10 | gb105pln | Delphinium exaltatum nuclear 26S ribosomal RNA gene, partial sequence. | |
| 700382816H1 | g288366 | 31 | −9 | gb105pln | Z. mays PGg14 gene for polygalacturonase. | |
| 700356282H1 | g19852 | 18 | 8 | gb105pln | N. tabacum mRNA for cytochrome b5 (partial). | |
| 700382937H1 | g549983 | 39 | −35 | gb105pln | Pennisetum ciliare possible apospory-associated mRNA clone pSUB C, complete cds. | |
| 700354672H1 | g971862 | 31 | −10 | gb105allp | potential laminin-binding protein | |
| 700382544H1 | g790582 | 16 | 9 | gb105pln | Arabidopsis thaliana putative pathogenesis-related protein (ATOZI1) mRNA, | |
| | | | | | complete cds. | |
| 700354368H1 | g2150129 | 26 | −6 | gb105pln | Arabidopsis thaliana cytoplasmic ribosomal protein S15a mRNA, complete cds. | |
| 700354623H1 | g2529228 | 33 | −31 | gb105pln | Glycine max mRNA for 6-phosphogluconate dehydrogenase, complete cds. | |
| 700382315H1 | g20321 | 70 | −62 | gb105pln | Oryza sativa RAc1 mRNA for actin. | |
| 700355492H1 | g313139 | 60 | −57 | gb105pln | Z. mays ZmPRO2 mRNA for profilin 2. | |
| 700355226H1 | g1167835 | 19 | 3 | gb105pln | H. lanatus mRNA for Hol lI protein. | |
| 700353959H1 | g2795809 | 14 | 0 | gb105allp | putative expansin | |
| 700356281H1 | g287829 | 84 | −54 | gb105pln | Z. mays gene for polygalacturonase. | |
| 700355048H1 | g2264318 | 32 | −6 | gb105pln | Arabidopsis thaliana genomic DNA chromosome 5, P1 clone: MUP24, complete | |
| | | | | | sequence. | |
| 700352990H1 | g535583 | 56 | −4 | gb105pln | Medicago sativa adenosylhomocysteinase mRNA, complete cds. | |
| 700382870H1 | g2267213 | 21 | −7 | gb105eukp | dynamin-like GTP binding protein | |
| 700382206H1 | g1550739 | 42 | −24 | gb105pln | A. thaliana mRNA for GDP dissociation inhibitor. | |
| 700383058H1 | g1524115 | 21 | −12 | gb105eukp | PR-P69; subtilisin-like endoprotease | |
| 700382231H1 | g1732518 | 25 | −10 | gb105pln | Arabidopsis thaliana Rho1Ps homolog mRNA, complete cds. | |
| 700382422H1 | g2827496 | 29 | −12 | gb105eukp | EG: 30B8.1 | |
| 700382486H1 | g2286150 | 63 | −69 | gb105pln | Zea mays translation initiation factor (eIF-4A) mRNA, complete cds. | |
| 700383157H1 | g2738247 | 32 | −18 | gb105pln | Arabidopsis thaliana cobalamin-independent methionine synthase (ATCIMS) mRNA, | |
| | | | | | complete cds. | |
| 700382875H1 | g22416 | 43 | −33 | gb105pln | Z. mays mRNA for polygalacturonase (clone PG1). | |
| 700382633H1 | g1107486 | 22 | −15 | gb105pln | A. thaliana mRNA for 60S ribosomal protein L27a. | |
| 700353891H1 | g1817578 | 30 | −22 | gb105allp | alpha-globin 2 | |
| 700354838H1 | g1002369 | 9 | 7 | gb105allp | coatomer protein | |
| 700355952H1 | g414907 | 30 | −11 | gb105pln | P. pratense mRNA for pollen allergen PhlpI. | |
| 700355995H1 | g1616656 | 32 | −21 | gb105pln | Arabidopsis thaliana adenylosuccinate synthetase mRNA, complete cds. | |
| 700382880H1 | g22418 | 62 | −37 | gb105pln | Z. mays gene for polygalacturonase (clone 17). | |
| 700352759H1 | g1209700 | 34 | −38 | gb105pln | Zea mays ribosomal protein L12 mRNA, complete cds. | |
| 700352750H1 | g2781363 | 24 | −4 | gb105eukp | F24O1.19 | |
| 700354805H1 | g348695 | 21 | −17 | gb105pln | Catharanthus roseus heat shock protein 90 mRNA, complete cds. | |
| 700356916H1 | g899607 | 87 | −31 | gb105pln | Zea mays polyubiquitin (MubC5) mRNA, complete cds. | |
| 700352708H1 | g1653962 | 10 | −2 | gb105allp | hypothetical protein | |
| 700356057H1 | g2218135 | 17 | 1 | gb105eukp | UDPGDH; UDP-glucose dehydrogenase | |
| 700354407H1 | g2642157 | 24 | −9 | gb105eukp | T5I7.5; ankyrin-like protein | |
| 700354747H1 | g406310 | 17 | −2 | gb105pln | B. napus (Topas) clpA mRNA. | |
| 700353930H1 | g21273 | 32 | 6 | gb105pln | Spinach mRNA for chloroplast phosphate translocator protein. | |
| 700354771H1 | g293902 | 6 | 0 | gb105eukp | Zea mI | |
| 700355229H1 | g22427 | 69 | −71 | gb105pln | Z. mays gene for polygalacturonase (clone 65). | |
| 700353141H1 | g286033 | 24 | −21 | gb105pln | Ipomoea batatas (sweet potato) mRNA for F1-ATPase gamma-subunit. | |
| 700352639H1 | g166687 | 27 | −2 | gb105pln | A. thaliana 3-deoxy-D-arabino-heptulosonate y-phosphate synthase (DHS1) mRNA, | |
| | | | | | complete cds. | |
| 700354639H1 | g1323704 | 14 | 5 | gb105allp | similar to C. elegans F38E1.9 gene product encoded by GenBank Accession Number | |
| | | | | | U41996 | |
| 700354906H1 | g169580 | 26 | −2 | gb105eukp | trypsin inhibitor precursor (put.); putative | |
| 700382428H1 | g473103 | 18 | −19 | gb105pln | N. tabacum mRNA for protein homologue to Human Wilm's tumor-related protein | |
| | | | | | HUMQM (partial). | |
| 700382390H1 | g22458 | 84 | −73 | gb105pln | Z. mays pollen specific mRNA C-terminal (clone 4H7). | |
| 700382845H1 | g395071 | 25 | −22 | gb105pln | V. faba guanine nucleotide regulatory protein mRNA, complete CDS. | |
| 700354795H1 | g2827700 | 12 | −4 | gb105eukp | F6H11.20; DEAD box ATP dependent helicase protein | |
| 700383048H1 | g2113805 | 19 | −7 | gb105eukp | AmphiP2; ribosomal protein P2 | |
| 700355693H1 | g1438883 | 15 | 5 | gb105allp | GmCK3p | |
| 700354886H1 | g533692 | 16 | 3 | gb105allp | protease inhibitor | |
| 700354725H1 | g2345147 | 28 | −40 | gb105pln | Pisum sativum developmentally regulated GTP binding protein (PsDRG1) mRNA, | |
| | | | | | complete cds. | |
| 700352641H1 | g799376 | 16 | −4 | gb105pln | Zea mays beta-D-glucosidase mRNA, nuclear gene encoding chloroplast protein, | |
| | | | | | complete cds. | |
| 700383182H1 | g1296806 | 17 | −25 | H. vulgare xylose isomerase gene. | | |
| 700352695H1 | g1895083 | 9 | −8 | gb105pln | Zea mays golgi associated protein se-wap41 mRNA, complete cds. | |
| 700382315H1 | g1498391 | 40 | −75 | gb105pln | Zea mays actin (Maz65) gene, partial cds. | |
| 700282406H2 | g313137 | 23 | −5 | gb105pln | Z. mays ZmPRO1 mRNA for profilin 1. | |
| 700353922H1 | g1212995 | 63 | −51 | gb105pln | H. vulgare mRNA for UDP-glucose pyrophosphorylase. | |
| 700352687H1 | g20726 | 39 | −26 | gb105pln | P. sativum GA mRNA (clone F). | |
| 700353171H1 | g1771781 | 41 | −0 | gb105pln | T. aestivum mRNA for beta-amylase. | |
| 700383114H1 | g22416 | 84 | −70 | gb105pln | Z. mays mRNA for polygalacturonase (clone PG1). | |
| 700352766H1 | g488574 | 25 | −6 | gb105pln | Medicago sativa Regen S clone pH3c130 histone H3.2 mRNA, complete cds. | |
| 700354319H1 | g1345131 | 12 | 14 | gb105pln | Arabidopsis thaliana ERECTA mRNA, complete cds. | |
| 700355862H1 | g313141 | 72 | −81 | gb105pln | Z. mays ZmPRO3 mRNA for profilin 3. | |
| 700353695H1 | g168313 | 18 | 2 | gb105pln | L. perenne pollen allergen (Lol pI) gene, 3′ end. | |
| 700383090H1 | g287628 | 48 | −8 | gb105eukp | YS29A; ribosomal protein YS29 | |
| 700354562H1 | g1546918 | 27 | −1 | gb105pln | Z. mays mRNA for translation initiation factor 5A. | |
| 700352918H1 | g1167836 | 34 | −0 | gb105eukp | hol lI; major group I allergen; protein with incomplete signal sequence | |
| 700354175H1 | g886400 | 37 | −16 | gb105pln | Oryza sativa MADS-box protein (MADS2) mRNA, complete cds. | |
| 700354619H1 | g529009 | 26 | −7 | gb105eukp | PFK; PPi-dependent phosphofructo-1-kinase; EC 2.7.1.90 | |
| 700354992H1 | g414702 | 20 | −15 | gb105pln | H. lanatus mRNA for allergen Hol-lI. | |
| 700354410H1 | g18887 | 31 | −31 | gb105pln | H. vulgare agpp mRNA for ADP-glucose pyrophosphorylase small subunit. | |
| 700353255H1 | g1345548 | 6 | 2 | gb105eukp | NPG; polygalacturonase; EC 3.2.1.15 | |
| 700356127H1 | g435648 | 38 | −28 | gb105pln | Rice mRNA for gamma-Tip, complete cds. | |
| 700353549H1 | g168460 | 79 | −6 | gb105pln | Zea mays cyclophilin (CyP) mRNA, complete cds. | |
| 700353627H1 | g2245512 | 13 | −10 | gb105eukp | protein phosphatase-1 | |
| 700353918H1 | g22416 | 35 | −27 | gb105pln | Z. mays mRNA for polygalacturonase (clone PG1). | |
| 700356188H1 | g2398679 | 14 | 3 | gb105eukp | DS1; 3-deoxy-D-arabino-heptulosonate 7-phosphate syntahse; EC 4.1.2.15 | |
| 700355951H1 | g414702 | 19 | −2 | gb105pln | H. lanatus mRNA for allergen Hol-lI. | |
| 700354923H1 | g1532072 | 54 | −49 | gb105pln | Z. mays mRNA for S-adenosylmethionine decarboxylase. | |
| 700353539H1 | g2829275 | 45 | −6 | gb105eukp | ndpk3; nucleoside diphosphate kinase 3 | |
| 700354785H1 | g2262155 | 21 | −1 | gb105pln | DNA sequence of Arabidopsis thaliana BAC F5J6 from chromosome IV, complete | |
| | | | | | sequence. | |
| 700382129H1 | g1617268 | 17 | −3 | gb105eukp | acyl CoA synthetase; EC 6.2.1.3 | |
| 700354253H1 | g414702 | 27 | −12 | gb105pln | H. lanatus mRNA for allergen Hol-lI. | |
| 700355844H1 | g2108254 | 8 | 1 | gb105eukp | pgp2; P-glycoprotein-2 | |
| 700383036H1 | g1805580 | 14 | −1 | gb105allp | sensor-regulator protein barA | |
| 700354325H1 | g1498389 | 35 | −42 | gb105pln | Zea mays actin (Maz81) gene, partial cds. | |
| 700382072H1 | g2673867 | 24 | −4 | gb105pln | Antirrhinum majus mRNA for fimbriata-associated protein 1, partial. | |
| 700356880H1 | g288373 | 96 | −21 | gb105pln | Z. mays PGp6 gene for polygalacturonase. | |
| 700352754H1 | g1658192 | 78 | −65 | gb105pln | Sorghum bicolor obtusifoliol 14-alpha demethylase CYP51 (CYP51) mRNA, | |
| | | | | | complete cds. | |
| 700383070H1 | g2809251 | 7 | 7 | gb105eukp | F21B7.20 | |
| 700383039H1 | g20277 | 21 | 1 | gb105eukp | OC-II; oryzacystatin II | |
| 700356960H1 | g2389003 | 16 | 7 | gb105eukp | SPAC6F6.13c; hypothetical protein | |
| 700353954H1 | g2102693 | 26 | −8 | gb105eukp | Frk2; fructokinase; EC 2.7.1.4 | |
| 700357411H1 | g22178 | 34 | 5 | gb105pln | Z. mays 22 kD alpha-zein gene. | |
| 700355296H1 | g1749567 | 23 | 15 | gb105pln | Fission Yeast mRNA, partial cds. | |
| 700282418H2 | g22509 | 49 | −86 | gb105pln | Zea mays waxy (wx+) locus for UDP-glucose starch glycosyl transferase. | |
| 700352602H1 | g1142698 | 9 | 0 | gb105eukp | NADPH-dependent aldehyde reductase; EC 1.1.1.2 | |
| 700353151H1 | g166766 | 19 | −3 | gb105eukp | heat shock protein HSP70-2 | |
| 700356493H1 | g1129144 | 43 | −22 | gb105pln | M. indica (Manila THMF5 mRNA for 3-ketoacyl-coA thilase B. | |
| 700355331H1 | g1143864 | 16 | −5 | gb105eukp | catalyzes the release of either giberellin or cyanogenic substances from their | |
| | | | | | glucoconjugates; beta glucosidase; EC 3.2.1.21 | |
| 700352631H1 | g1532212 | 39 | 0 | gb105eukp | cypA; cyclophilin A | |
| 700354910H1 | g1871360 | 29 | −7 | gb105eukp | glutamyl-prolyl-tRNA synthetase | |
| 700356435H1 | g2668739 | 81 | −54 | gb105pln | Zea mays translation initiation factor GOS2 (TIF) mRNA, complete cds. | |
| 700355901H1 | g16511 | 22 | −3 | gb105pln | A. thaliana mRNA for suppressor-like protein. | |
| 700356461H1 | g17561 | 17 | 7 | gb105allp | PROTEINASE INHIBITOR | |
| 700355692H1 | g248336 | 32 | −38 | gb105pln | polyubiquitin [maize, Genomic, 3841 nt]. | |
| 700353141H1 | g2702261 | 16 | 12 | gb105pln | Arabidopsis thaliana chromosome II BAC T21L14 genomic sequence, complete | |
| | | | | | sequence. | |
| 700352918H1 | g414703 | 34 | −0 | gb105eukp | allergen Hol-lI | |
| 700357450H1 | g312180 | 25 | −34 | gb105pln | Z. mays GapC4 gene. | |
| 700354838H1 | g1237029 | 9 | 7 | gb105allp | alpha-cop protein | |
| 700356242H1 | g2511574 | 37 | 3 | gb105eukp | prc3; multicatalytic endopeptidase; EC 3.4.99.46 | |
| 700356511H1 | g288366 | 48 | −64 | gb105pln | Z. mays PGg14 gene for polygalacturonase. | |
| 700353026H1 | g1002531 | 44 | −0 | gb105eukp | ACT4; actin-4 | |
| 700352978H1 | g736271 | 63 | −5 | gb105pln | O. sativa hsp70 gene for heat shock protein 70. | |
| 700354472H1 | g177065 | 52 | −39 | gb105allp | beta-2-microglobulin | |
| 700354952H1 | g2160162 | 20 | 5 | gb105eukp | F21M12.8 | |
| 700356611H1 | g1049252 | 86 | −29 | gb105pln | Zea mays vacuolar ATPase 69 kDa subunit mRNA, partial cds. | |
| 700352602H1 | g450473 | 9 | 0 | gb105allp | steroid 3-alpha-dehydrogenase | |
| 700382630H1 | g2529657 | 11 | 17 | gb105pln | Arabidopsis thaliana chromosome II BAC T30B22 genomic sequence, complete | |
| | | | | | sequence. | |
| 700352825H1 | g2827552 | 14 | −6 | gb105eukp | T12H17.140; predicted protein | |
| 700354277H1 | g567226 | 34 | −15 | gb105allp | mATF4 | |
| 700382094H1 | g168608 | 46 | −75 | gb105pln | Maize 17S ribosomal RNA gene and flanks. | |
| 700355293H1 | g20323 | 24 | −20 | gb105pln | O. sativa RAc1 gene for actin. | |
| 700353106H1 | g432367 | 29 | −6 | gb105pln | Rice mRNA for elongation factor 1 beta, complete cds. | |
| 700382131H1 | g2832716 | 19 | −2 | gb105pln | Lolium temulentum mRNA for alkaline/neutral invertase. | |
| 700356242H1 | g2570504 | 23 | 8 | gb105pln | Oryza sativa proteasome component mRNA, complete cds. | |
| 700355693H1 | g1438879 | 14 | 7 | gb105eukp | GmCK1p; EC 2.7.1.32 | |
| 700383032H1 | g1002916 | 15 | 6 | gb105pln | Oryza sativa cap-binding protein p28 mRNA, complete cds. | |
| 700355223H1 | g435172 | 46 | −54 | gb105pln | A. sativa (Pewi) ASTCP-K19 mRNA for t complex polypeptide 1. | |
| 700352784H1 | g171809 | 19 | −1 | gb105eukp | RPL10E | |
| 700354427H1 | g2190418 | 20 | 4 | gb105pln | Lycopersicon esculentum mRNA for Dem protein. | |
| 700352764H1 | g829147 | 86 | −72 | gb105pln | Z. mays gene for cyclophilin. | |
| 700355311H1 | g312177 | 23 | −5 | gb105eukp | RPL2; ribosomal protein L2 | |
| 700356089H1 | g1103317 | 32 | −18 | gb105pln | A. thaliana CKI1 mRNA for casein kinase I. | |
| 700353225H1 | g927571 | 87 | −80 | gb105pln | Z. mays mRNA for calreticulin precursor. | |
| 700354709H1 | g2330689 | 14 | −1 | gb105pln | Yeast (Saccharomyces pombe) chromosome I cosmid c16E8. | |
| 700352717H1 | g1323077 | 5 | 7 | gb105eukp | ERG25 | |
| 700383038H1 | g347062 | 39 | −19 | gb105eukp | RPL37A; ribosomal protein | |
| 700353418H1 | g22425 | 94 | −42 | gb105pln | Z. mays gene for polygalacturonase (clone 47). | |
| 700356907H1 | g777757 | 57 | −20 | gb105pln | Saccharum hybrid (clone SCUBI561) polyubiquitin mRNA, complete cds. | |
| 700354920H1 | g22418 | 76 | −47 | gb105pln | Z. mays gene for polygalacturonase (clone 17). | |
| 700352629H1 | g473999 | 24 | 2 | gb105pln | Rice mRNA, partial homologous to ribosomal protein L31 gene. | |
| 700357440H1 | g1752735 | 13 | 15 | gb105pln | Yeast DNA for MNN4 gene, complete cds. | |
| 700382815H1 | g2160158 | 19 | 4 | gb105allp | Similar to elongation factor 1-gamma (gb\|EF1G_XENLA). ESTs gb\|T20564, gb\|T45940, | |
| | | | | | gb\|T04527 come from this gene. | |
| 700352974H1 | g2352553 | 77 | −6 | gb105eukp | Hsp82; 82 kDa heat shock protein | |
| 700354555H1 | g857573 | 28 | 14 | gb105pln | Oryza sativa vacuolar H+-ATPase (vatp-P1) mRNA, complete cds. | |
| 700353240H1 | g473602 | 50 | −33 | gb105pln | Zea mays W-22 histone H2A mRNA, complete cds. | |
| 700352939H1 | g1020415 | 12 | 5 | gb105allp | phospholipase D | |
| 700355330H1 | g1800214 | 64 | −70 | gb105pln | Sorghum bicolor phytochrome A (PHYA) mRNA, complete cds. | |
| 700355955H1 | g13011 | 24 | 1 | gb105allp | URF 3 (NADH dehydrogenase subunit) | |
| 700382005H1 | g22157 | 71 | −83 | gb105pln | Z. mays mRNA for alpha-tubulin 6. | |
| 700382818H1 | g758354 | 61 | −61 | gb105pln | Z. mays mRNA for plasma membrane H+ ATPase. | |
| 700352990H1 | g2244747 | 57 | −4 | gb105pln | Arabidopsis thaliana DNA chromosome 4, ESSA I contig fragment No. 0. | |
| 700353952H1 | g349079 | 14 | 6 | gb105allp | growth response protein | |
| 700353952H1 | g349079 | 14 | 6 | gb105allp | growth response protein | |
| 700356516H1 | g2302292 | 20 | −3 | gb105allp | unnamed protein product | |
| 700353514H1 | g2829894 | 28 | −8 | gb105eukp | T26J12.6 | |
| 700383041H1 | g2351066 | 23 | −5 | gb105pln | Arabidopsis thaliana genomic DNA, chromosome 5, P1 clone: MOP9. | |
| 700353236H1 | g1931639 | 10 | −1 | gb105eukp | T19D16.3; lysophospholipase isolog | |
| 700383132H1 | g414908 | 21 | 2 | gb105eukp | pollen allergen Phl pI | |
| 700353081H1 | g2852449 | 15 | 6 | gb105allp | protein kinase | |
| 700353090H1 | g1666172 | 30 | −8 | gb105pln | N. plumbaginifolia mRNA for BTF3-like transcription factor. | |
| 700382306H1 | g531388 | 20 | −2 | gb105pln | Zea mays copia-like retrotransposon Hopscotch polyprotein (gag/pol) genes, | |
| | | | | | complete cds. | |
| 700354490H1 | g1504051 | 12 | −10 | gb105pln | Maize; corn mRNA for Calcium-dependent protein kinase, complete cds. | |
| 700354642H1 | g804944 | 8 | 7 | gb105allp | wpk4 protein kinase | |
| 700355818H1 | g2224914 | 28 | −12 | gb105pln | Oryza sativa beta-expansin mRNA, complete cds. | |
| 700355072H1 | g666976 | 49 | −24 | gb105pln | A. thaliana TYKY mRNA for NADH:ubiquinone oxidoreductase. | |
| 700355573H1 | g19848 | 21 | −11 | gb105pln | N. tabacum mRNA for pre-pro-cysteine proteinase. | |
| 700382957H1 | g22416 | 28 | −29 | gb105pln | Z. mays mRNA for polygalacturonase (clone PG1). | |
| 700382935H1 | g1016290 | 10 | 7 | gb105eukp | NAB4; nuclear polyadenylated RNA-binding protein | |
| 700354991H1 | g1218004 | 48 | −1 | gb105eukp | cell plate formation; SDL5A | |
| 700355860H1 | g2624416 | 25 | −26 | gb105pln | Zea mays mRNA for ubiquitin carrier protein UBC7. | |
| 700352972H1 | g255569 | 21 | 4 | gb105eukp | pollen specific protein | |
| 700352707H1 | g425193 | 28 | −41 | gb105pln | Spinacia oleracea heat shock C70 protein mRNA, complete cds. | |
| 700356205H1 | g407524 | 21 | −6 | gb105pln | S. cereale tpi gene for triosephosphate isomerase. | |
| 700355936H1 | g688011 | 6 | 4 | gb105allp | AgX-1 antigen [human, infertile patient, testis, Peptide, 505 aa] | |
| 700356101H1 | g499011 | 38 | −27 | gb105pln | S. vulgare SoAc1 mRNA. | |
| 700355284H1 | g22458 | 17 | −27 | gb105pln | Z. mays pollen specific mRNA C-terminal (clone 4H7). | |
| 700355911H1 | g397958 | 36 | −53 | gb105pln | Z. mays mRNA for beta-amylase. | |
| 700353726H1 | g303854 | 24 | 11 | gb105pln | Rice mRNA for ribosomal protein L7A, complete cds. | |
| 700355533H1 | g2529341 | 26 | −25 | gb105pln | Spinacia oleracea chloroplast transketolase mRNA, complete cds. | |
| 700383022H1 | g473359 | 25 | −15 | gb105pln | P. pratense mRNA for Phl pI allergen. | |
| 700353994H1 | g1724101 | 53 | −15 | gb105pln | Mesembryanthemum crystallinum S-adenosyl-L-homocystein hydrolase mRNA, | |
| | | | | | complete cds. | |
| 700356028H1 | g2331300 | 63 | −29 | gb105pln | Zea mays ribosomal protein S4 type I (rps4) mRNA, complete cds. | |
| 700354893H1 | g460044 | 18 | −7 | gb105eukp | FUN12; Fun12p | |
| 700353286H1 | g1008444 | 24 | −25 | gb150pln | T. aestivum mRNA for profilin (clone TaPRO3). | |
| 700355237H1 | g1203831 | 15 | 8 | gb105pln | Hordeum vulgare beta-D-glucan exohydrolase, isoenzyme ExoII, mRNA, complete cds. | |
| 700356746H1 | g168314 | 23 | 8 | gb105eukp | Lol pI; pollen allergen | |
| 700382074H1 | g1184773 | 47 | −71 | gb105pln | Zea mays glyceraldehyde-3-phosphate dehydrogenase GAPC3 (gpc3) mRNA, | |
| | | | | | complete cds. | |
| 700352769H1 | g606814 | 38 | −20 | gb105pln | Zea mays Golden Bantam carbonic anhydrase mRNA, complete cds. | |
| 700352812H1 | g1289203 | 14 | −24 | gb105pn | A. glutinos mRNA for thiazole biosynthetic enzyme. | |
| 700282554H2 | g2463588 | 10 | 4 | gb105eukp | FB1035 precursor | |
| 700382110H1 | g1167835 | 10 | 5 | gb105pln | H. lanatus mRNA for Hol lI protein. | |
| 700282461H2 | g1769814 | 18 | 2 | gb105allp | beta-D-glucosidase | |
| 700354922H1 | g1769898 | 7 | 6 | gb105eukp | lectin receptor kinase | |
| 700354833H1 | g602252 | 17 | −0 | gb105pln | Zea mays enolase (eno2) mRNA, complete cds. | |
| 700353995H1 | g287829 | 80 | −7 | gb105pln | Z. mays gene for polygalacturonase. | |
| 700355741H1 | g1906829 | 20 | 5 | gb105pln | A. thaliana hsp88.1 gene. | |
| 700353037H1 | g1675393 | 52 | −24 | gb105pln | Oryza sativa class III ADH enzyme (AdhIII) gene, complete cds. | |
| 700382221H1 | g288611 | 88 | −61 | gb105pln | Z. mays PG gene for polygalacturonoase. | |
| 700282518H2 | g2656030 | 35 | −11 | gb105pln | Arabidopsis thaliana genomic DNA, chromosome 5, P1 clone: MUL8. | |
| 700354501H1 | g217963 | 67 | −27 | gb105pln | Zea mays mRNA for ribulose 1,5-bisphosphate carboxylase small subunit, complete cds. | |
| 700382349H1 | g2264305 | 14 | 15 | gb105pln | Arabidopsis thaliana genomic DNA, chromosome 5, P1 clone: MBK23, complete | |
| | | | | | sequence. | |
| 700354604H1 | g973408 | 6 | 4 | gb105eukp | ppx1; PPX homolog | |
| 700355659H1 | g288373 | 21 | −11 | gb105pln | Z. mays PGg6 gene for polygalacturonase. | |
| 700356507H1 | g312180 | 30 | −18 | gb105pln | Z. mays GapC4 gene. | |
| 700353177H1 | g2688821 | 14 | −14 | gb105pln | Prunus armeniaca pyrophosphate-dependent phosphofructo-1-kinase mRNA, partial cds. | |
| 700352759H1 | g474003 | 22 | 7 | gb105pln | Rice mRNA, partial homologous to ribosomal protein rp21c gene. | |
| 700382824H1 | g902583 | 66 | −73 | gb105pln | Zea mays clone MubG1 ubiquitin gene, complete cds. | |
| 700382439H1 | g63752 | 12 | 6 | gb105allp | PR 264 | |
| 700383178H1 | g2351198 | 46 | −24 | gb105allp | PA28 beta subunit | |
| 700382110H1 | g19495 | 9 | 12 | gb105pln | L. perenne RNA for Lol pI gene. | |
| 700382001H1 | g2435511 | 22 | −1 | gb105eukp | A_TM017A05.10 | |
| 700352620H1 | g168608 | 100 | −85 | gb105pln | Maize 17S ribosomal RNA gene and flanks. | |
| 700354472H1 | g179314 | 52 | −39 | gb105allp | beta-2-microglobulin | |
| 700353779H1 | g1835730 | 11 | 17 | gb105pln | Oryza sativa photosystem II 10 kDa polypeptide mRNA, complete cds. | |
| 700356069H1 | g395071 | 60 | 14 | gb105pln | V. faba guanine nucleotide regulatory protein mRNA, complete CDS. | |
| 700355816H1 | g396148 | 23 | 4 | gb105pln | Z. mays CHI gene. | |
| 700382937H1 | g531481 | 39 | −35 | gb105pln | P. ciliare (Higgins) apospory associated mRNA, 1398 bp. | |
| 700355281H1 | g600768 | 30 | 7 | gb105pln | Oryza sativa cyclophilin 2 (Cyp2) mRNA, complete cds. | |
| 700353568H1 | g1304270 | 37 | 3 | gb105eukp | zha1; plasma membrane H+-ATPase; EC 3.6.1.35 | |
| 700382961H1 | g170775 | 18 | −33 | gb105pln | Wheat translation elongation factor 1 alpha-subunit (TEF1) mRNA, complete cds. | |
| 700353857H1 | g576509 | 19 | −1 | gb105eukp | IAP86; component of chloroplast outer membrane protein import apparatus; GTP-binding | |
| | | | | | protein | |
| 700352813H1 | g2632156 | 13 | −8 | gb105pln | Aspergillus niger mRNA for ribosomal protein L15. | |
| 700354542H1 | g2345153 | 45 | −63 | gb105pln | Zea mays ribsomal protein S4 (rps4) mRNA, complete cds. | |
| 700355589H1 | g2190558 | 14 | −8 | gb105eukp | F5I14.14; F5I14.14 | |
| 700382877H1 | g473359 | 25 | 0 | gb105pln | P. pratense mRNA for Phl pI allergen. | |
| 700353163H1 | g968901 | 64 | 11 | gb105pln | Rice mRNA for ribosomal protein S8, complete cds. | |
| 700352718H1 | g469247 | 21 | 2 | gb105pln | Helianthus annuus ribosomal protein S3a mRNA, complete cds. | |
| 700382280H1 | g2505864 | 19 | 2 | gb105pln | Arabidopsis thaliana DNA, 24 kb surrounding PFL locus. | |
| 700354514H1 | g288366 | 55 | −29 | gb105pln | Z. mays PGg14 gene for polygalacturonase. | |
| 700356849H1 | g2275201 | 6 | 8 | gb105eukp | T08I13.7 | |
| 700355629H1 | g20321 | 32 | −31 | gb105pln | Oryza sativa RAc1 mRNA for actin. | |
| 700382596H1 | g431270 | 25 | −7 | gb105eukp | UBC5; ubiquitin conjugating enzyme | |
| 700356112H1 | g886400 | 67 | −61 | gb105pln | Oryza sativa MADS-box protein (MADS2) mRNA, complete cds. | |
| 700382068H1 | g849195 | 6 | 6 | gb105eukp | YDR374C; Ydr374cp | |
| 700354874H1 | g777757 | 28 | −24 | gb105pln | Saccharum hybrid (clone SCUBI561) polyubiquitin mRNA, complete cds. | |
| 700353875H1 | g313137 | 59 | −67 | gb105pln | Z. mays ZmPRO1 mRNA for profilin 1. | |
| 700354611H1 | g1907211 | 9 | 6 | gb105eukp | YCR072c, len:515 | |
| 700354657H1 | g1906825 | 47 | −31 | gb105pln | A. thaliana hsp81.2 gene. | |
| 700355629H1 | g499011 | 33 | −33 | gb105pln | S. vulgare SoAc1 mRNA. | |
| 700354412H1 | g288373 | 56 | −17 | gb105pln | Z. mays PGg6 gene for polygalacturonase. | |
| 700354690H1 | g22423 | 63 | −49 | gb105pln | Z. mays mRNA for polygalacturonase (clone PG4). | |
| 700354734H1 | g288611 | 91 | −82 | gb105pln | Z. mays PG gene for polygalacturonase. | |
| 700352629H1 | g406833 | 34 | 3 | gb105allp | 60S ribosoma protein L31 | |
| 700354786H1 | g19495 | 12 | 2 | gb105pln | L. perenne RNA for Lol pI gene. | |
| 700352929H1 | g2337876 | 50 | −9 | gb105allp | human PH-20 homolog (LUCA-2) | |
| 700355331H1 | g804656 | 23 | −9 | gb105eukp | BGQ60; beta-glucosidase | |
| 700282533H2 | g303733 | 39 | −31 | gb105pln | Pea mRNA for GTP-binding protein, complete cds. | |
| 700382615H1 | g2655294 | 24 | −6 | gb105pln | Oryza sativa germin-like protein 6 (GER6) mRNA, complete cds. | |
| 700282523H2 | g168314 | 16 | 7 | gb105eukp | Lol pI; pollen allergen | |
| 700356827H1 | g606969 | 19 | −4 | gb105pln | Arabidopsis thaliana cytoplasmic ribosomal protein L18 mRNA, complete cds. | |
| 700353263H1 | g1906825 | 54 | −53 | gb105pln | A. thaliana hsp81.2 gene. | |
| 700352940H1 | g16427 | 15 | 8 | gb105allp | protease inhibitor II | |
| 700354831H1 | g897716 | 21 | −3 | gb105eukp | ZC504.4 | |
| 700353610H1 | g2668739 | 44 | −83 | gb105pln | Zea mays translation initiation factor GOS2 (TIF) mRNA, complete cds. | |
| 700356567H1 | g1911581 | 42 | −12 | gb105pln | Cyn d a = major allergen {clone 14c1 and CD1} [Cynodon dactylon = Bermuda grass, | |
| | | | | | pollen, mRNA Partial, 759 nt]. | |
| 700355459H1 | g886470 | 52 | −40 | gb105pln | C. roseus MetE mRNA for methionine synthase. | |
| 700382336H1 | g433040 | 22 | 14 | gb105pln | Zea mays W-22 clone PREM-1A retroelement PREM-1, partial sequence. | |
| 700356517H1 | g2314803 | 27 | −10 | gb105pln | Oryza sativa signal recognition particle RNA sequence. | |
| 700356057H1 | g1518540 | 18 | 1 | gb105allp | UDP-glucose dehydrogenase | |
| 700354929H1 | g473359 | 23 | −6 | gb105pln | P. pratense mRNA for Phl pI allergen | |
| 700352905H1 | g2655388 | 34 | −9 | gb105eukp | Cytb; cytochrome b | |
| 700352838H1 | g2827552 | 20 | 2 | gb105eukp | T12H17.140; predicted protein | |
| 700382956H1 | g550543 | 23 | −2 | gb105pln | A. thaliana mRNA for ribosomal protein L16. | |
| 700354253H1 | g414907 | 27 | −12 | gb105pn | P. pratense mRNA for pollen allergen Phl pI. | |
| 700382544H1 | g790583 | 29 | −4 | gb105eukp | ATOZI1 | |
| 700353963H1 | g22416 | 78 | −12 | gb105pln | Z. mays mRNA for polygalacturonase (clone PG1). | |
| 700382946H1 | g20323 | 37 | −29 | gb105pln | O. sativa RAc1 gene for actin. | |
| 700353002H1 | g296447 | 12 | 4 | gb105eukp | unnamed protein product | |
| 700354909H1 | g288373 | 69 | −62 | gb105pln | Z. mays PGg6 gene for polygalacturonase. | |
| 700356750H1 | g288611 | 87 | −34 | gb105pln | Z. mays PG gene for polygalacturonase. | |
| 700355446H1 | g17782 | 27 | 1 | gb105eukp | Bplo | |
| 700352674H1 | g169435 | 41 | −27 | gb105pln | P. agilis beta-2 tubulin (beta-2-tub) gene, complete cds. | |
| 700354380H1 | g310934 | 28 | 0 | gb105pln | Nicotiana tabacum ribosomal protein L25, complete cds. | |
| 700355839H1 | g1872162 | 19 | −17 | gb105pln | Arabidopsis thaliana DnaJ homolog (atj) mRNA, complete cds. | |
| 700356010H1 | g463269 | 10 | 8 | gb105eukp | YBL0410 | |
| 700356675H1 | g2501763 | 36 | −2 | gb105pln | Glycine max calmodulin-like domain protein kinase isoenzyme beta mRNA, | |
| | | | | | complete cds. | |
| 700356230H1 | g218082 | 40 | −9 | gb105pln | Rice mRNA for initiation factor eIF-4D (225 gene), partial sequence. | |
| 700383160H1 | g600768 | 36 | −34 | gb105pln | Oryza sativa cyclophilin 2 (Cyp2) mRNA, complete cds. | |
| 700382435H1 | g288373 | 78 | −4 | gb105pln | Z. mays PGg6 gene for polygalacturonase. | |
| 700382256H1 | g170240 | 40 | −20 | gb105pln | Tobacco (N. tabacum) GapC mRNA encoding cytosolic glyceraldehyde-3-phosphophate | |
| | | | | | dehydrogenase, partial cds. | |
| 700382171H1 | g2511690 | 17 | 7 | gb105pln | Phaseolus vulgaris Moldavian encoding cysteine proteinase precursor (clone cp41). | |
| 700352717H1 | g2827552 | 18 | −1 | gb105eukp | T12H17.140; predicted protein | |
| 700382067H1 | g1806139 | 25 | −16 | gb105pln | M. sativa mRNA for cdc2 kinase homologue, cdc2MsC. | |
| 700355778H1 | g2138315 | 13 | 0 | gb105pln | Arabidopsis thaliana root hair defective 3 (RHD3) gene, complete cds. | |
| 700353102H1 | g287829 | 96 | −59 | gb105pln | Z. mays gene for polygalacturonase. | |
| 700282469H2 | g288378 | 64 | −87 | gb105pln | Z. mays gene PG for polygalacturonase. | |
| 700382445H1 | g602564 | 19 | −14 | gb105pln | C. paradisi (Macf) INO1 gene. | |
| 700382715H1 | g1262579 | 27 | −0 | gb105allp | NADH dehydrogenase subunit 1 | |
| 700353161H1 | g22458 | 17 | −1 | gb105pln | Z. mays pollen specific mRNA C-terminal (clone 4H7). | |
| 700353531H1 | g493621 | 16 | −3 | gb105pln | A. thaliana mRNA for chloroplast envelope Ca2+-ATPase, complete cds. | |
| 700383122H1 | g1665777 | 7 | 5 | gb105allp | Similar to S. cerevisiae EMP70 protein precursor (S25110) | |
| 700355742H1 | g836779 | 10 | 5 | gb105allp | YFR024C | |
| 700353742H1 | g348717 | 26 | −8 | gb105pln | Medicago truncatula protochlorophyllide reductase homolgue protein mRNA, | |
| | | | | | complete cds. | |
| 700356668H1 | g218088 | 57 | −17 | gb105pln | Rice mRNA for ribosomal protein 117 (249 gene), partial sequence. | |
| 700382348H1 | g170286 | 21 | −15 | gb105pln | Tobacco phytochrome B (phyB) mRNA, 3′ end. | |
| 700353054H1 | g294639 | 12 | 2 | gb105allp | sulfite oxidase | |
| 700353255H1 | g1345549 | 6 | 2 | gb105eukp | NPG; polygalacturonase; EC 3.2.1.15 | |
| 700354535H1 | g2431768 | 41 | −45 | gb105pln | Zea mays acidic ribosomal protein P1a (rpp1a) mRNA, complete cds. | |
| 700355728H1 | g2623295 | 11 | 2 | gb105eukp | T20B5.1 | |
| 700356208H1 | g313139 | 65 | −21 | gb105pln | Z. mays ZmPRO2 mRNA for profilin 2. | |
| 700355168H1 | g2809386 | 26 | −25 | gb105pln | Petroselinum crispum NADPH cytochrome P450 reductase (CPR1) mRNA, | |
| | | | | | complete cds. | |
| 700382542H1 | g1899187 | 21 | −14 | gb105pln | Nicotiana tabacum DNA binding protein ACBF mRNA, complete cds. | |
| 700354973H1 | g2198791 | 7 | −1 | gb105allp | phosphatidylinositol 4-kinase | |
| 700353054H1 | g540487 | 7 | 8 | gb105allp | nitrate reductase | |
| 700382687H1 | g699510 | 22 | −7 | gb105eukp | M(1)1B; minute(1)1B protein; minute(1)1B | |
| 700354931H1 | g22416 | 96 | −60 | gb105pln | Z. mays mRNA for polygalacturonase (clone PG1). | |
| 700354938H1 | g313141 | 49 | −33 | gb105pln | Z. mays ZmPRO3 mRNA for profilin 3. | |
| 700353202H1 | g1289203 | 43 | −36 | gb105pln | A. glutinosa mRNA for thiazole biosynthetic enzyme. | |
| 700353562H1 | g22314 | 94 | −47 | gb105pln | Maize mRNA for GSH gluthathione S-transferase I (GST; EC 2.5.1.18). | |
| 700382817H1 | g1749610 | 8 | 6 | gb105eukp | similar to Saccharomyces cerevisiae unknown, EMBL Accession Number Z68195 | |
| 700356086H1 | g540581 | 9 | 3 | gb105eukp | mudrA | |
| 700354373H1 | g476154 | 36 | −23 | gb105pln | Porphyra purpurea elongation factor EF1-alpha (tef-c) mRNA, complete cds. | |
| 700382314H1 | g312180 | 31 | −54 | gb105pln | Z. mays GapC4 gene. | |
| 700282456H2 | g1147612 | 26 | −18 | gb105eukp | MOT1; Mot1p: a putative helicase | |
| 700382221H1 | g288378 | 75 | −51 | gb105pln | Z. mays gene PG for polygalacturonase. | |
| 700382025H1 | g2529657 | 15 | 7 | gb105pln | Arabidopsis thaliana chromosome II BAC T30B22 genomic sequence, complete | |
| | | | | | sequence. | |
| 700355296H1 | g1749568 | 43 | 4 | gb105allp | similar to Saccharomyces cerevisiae eukaryotic initiation factor 4F subunit P130, | |
| | | | | | SWISS-PROT Accession Number P39936 | |
| 700356240H1 | g14264 | 59 | −61 | gb105pln | T. aestivum gene for sedoheptulose-1,7-bisphoshatase. | |
| 700356188H1 | g166690 | 10 | 2 | gb105allp | 3-deoxy-D-arabino-heptulosonate 7-phosphate synthase | |
| 700352844H1 | g1296806 | 28 | −23 | gb105pln | H. vulgare xylose isomerase gene. | |
| 700354926H1 | g2696803 | 18 | −2 | gb105pln | Oryza sativa mRNA for water channel protein, complete cds. | |
| 700354351H1 | g340368 | 15 | 2 | gb105allp | transfer RNA-Trp synthetase | |
| 700383004H1 | g1591622 | 10 | 3 | gb105allp | histidinol-phosphate aminotransferase (hisC) | |
| 700382339H1 | g16210 | 55 | −47 | gb105pln | Arabidopsis thaliana calnexin homolog. | |
| 700382252H1 | g1946361 | 8 | 6 | gb105eukp | T06B20.7 | |
| 700356667H1 | g1399567 | 59 | −18 | gb105pln | Podophyllum peltatum nuclear 26S ribosomal RNA gene, partial sequence. | |
| 700282591H2 | g1743378 | 23 | 5 | gb105allp | 3-hydroxy-3-methylglutaryl CoA lyase | |
| 700356209H1 | g312180 | 26 | −1 | gb105pln | Z. mays GapC4 gene. | |
| 700354660H1 | g606969 | 27 | −9 | gb105pln | Arabidopsis thaliana cytoplasmic ribosomal protein L18 mRNA, complete cds. | |
| 700356122H1 | g16379 | 17 | −8 | gb105pln | A. thaliana mRNA for laminin receptor homologue. | |
| 700353213H1 | g22416 | 79 | −68 | gb105pln | Z. mays mRNA for polygalacturonase (clone PG1). | |
| 700352636H1 | g1302311 | 6 | 8 | gb105eukp | ATX1 | |
| 700353611H1 | g21840 | 41 | −34 | gb105pln | T. aestivum RNA for phosphoribulokinase. | |
| 700352701H1 | g1794223 | 37 | −19 | gb105allp | DNA ligase III-alpha | |
| 700354754H1 | g289658 | 42 | −14 | gb105eukp | C50C3.6 protein | |
| 700354341H1 | g303854 | 45 | −59 | gb105pln | Rice mRNA for ribosomal protein L7A, complete cds. | |
| 700353549H1 | g829147 | 98 | −15 | gb105pln | Z. mays gene for cyclophilin. | |
| 700382005H1 | g450292 | 62 | −74 | gb105pln | Zea mays alpha-tubulin mRNA, complete cds. | |
| 700355010H1 | g485376 | 79 | −57 | gb105pln | Zea mays alpha-3-tubulin gene, complete cds. | |
| 700382393H1 | g2293565 | 25 | −0 | gb105pln | Oryza sativa ADP-ribosylation factor 1 (Os-ARF1) mRNA, complete cds. | |
| 700382166H1 | g577611 | 75 | −73 | gb105pln | zea mays CRT1 gene for clacium-binding protein. | |
| 700355780H1 | g517349 | 21 | 6 | gb105allp | metallothionein Ih | |
| 700382957H1 | g287829 | 28 | −28 | gb105pln | Z. mays gene for polygalacturonase. | |
| 700352942H1 | g2653284 | 57 | −36 | gb105pln | Oryza sativa mRNA for enoyl-ACP reductase. | |
| 700352929H1 | g1209016 | 50 | −9 | gb105allp | PH-20 homolog, similar to GenBank Accession Number S67798 | |
| 700282504H2 | g965472 | 16 | 6 | gb105allp | glutaredoxin-like protein | |
| 700354492H1 | g4790 | 27 | −8 | gb105eukp | YKL253 | |
| 700382232H1 | g1864023 | 24 | −3 | gb105pln | Cynodon dactylon calcium-binding pollen allergen gene, partial cds. | |
| 700282576H2 | g1749516 | 38 | −24 | gb105eukp | similar to Saccharomyces cerevisiae ornithine-oxo-acid aminotransferase, SWISS-PROT | |
| | | | | | Accession Number P07991 | |
| 700357426H1 | g2415406 | 8 | 4 | gb105allp | YodT | |
| 700383115H1 | g2055419 | 22 | −7 | gb105allp | ribosomal protein S12 | |
| 700382596H1 | g4321266 | 25 | −4 | gb105eukp | UBC4; ubiquitin conjugating enzyme | |
| 700382101H1 | g2245378 | 25 | −7 | gb105eukp | ARF1; auxin response factor 1 | |
| 700356777H1 | g1778820 | 65 | −23 | gb105pln | Oryza sativa S-adenosyl-L-methionine synthetase (pOS-SAMS2) mRNA, complete cds. | |
| 700356111H1 | g168460 | 39 | −79 | gb105pln | Zea mays cyclophilin (CyP) mRNA, complete cds. | |
| 700355217H1 | g288373 | 21 | 1 | gb105pln | Z. mays PGg6 gene for polygalacturonase. | |
| 700354675H1 | g1016136 | 21 | −3 | gb105eukp | rp15; ribosomal protein L5 | |
| 700382035H1 | g1755008 | 34 | −62 | gb105pln | Triticum aestivum calmodulin TRaCaM4-1 mRNA, complete cds. | |
| 700383175H1 | g473359 | 25 | −9 | gb105pln | P. pratense mRNA for Phl pI allergen. | |
| 700354342H1 | g218510 | 11 | 15 | gb105pln | Yeast genomic DNA for ribosomal protein YL16, complete cds. | |
| 700354944H1 | g1185555 | 44 | −38 | gb105pln | Zea mays glyceraldehyde-3-phosphate dehydrogenase (gpc4) gene, partial cds. | |
| 700352870H1 | g414702 | 46 | −33 | gb105pln | H. lanatus mRNA for allergen Hol-lI. | |
| 700354724H1 | g3075 | 18 | 1 | gb105pln | N. crassa mRNA for a ribosomal protein. | |
| 700353113H1 | g288366 | 95 | −79 | gb105pln | Z. mays PGg14 gene for polygalacturonase. | |
| 700356242H1 | g2570505 | 35 | 3 | gb105allp | proteasome component | |
| 700357429H1 | g602605 | 92 | −56 | gb105pln | Zea mays tandem genes for alpha1-tubulin and alpha2-tubulin. | |
| 700353162H1 | g288366 | 51 | −58 | gb105pln | Z. mays PGg14 gene for polygalacturonase. | |
| 700382101H1 | g2262117 | 24 | −2 | gb105eukp | T19F06.20; auxin inducible protein isolog | |
| 700354734H1 | g288378 | 76 | −69 | gb105pln | Z. mays gene PG for polygalacturonase. | |
| 700382033H1 | g1568635 | 22 | 4 | gb105eukp | host cell receptor involved in nuclear import of Agrobacterium VirD2 protein; | |
| | | | | | AtKAP alpha | |
| 700356821H1 | g1498325 | 33 | −40 | gb105pln | Glycine max actin (Soy86) gene, partial cds. | |
| 700356229H1 | g939780 | 36 | −29 | gb105pln | Zea mays MADS-box protein (ZAG5) mRNA, complete cds. | |
| 700354427H1 | g2190419 | 39 | −16 | gb105eukp | dem | |
| 700353525H1 | g288366 | 51 | −58 | gb105pln | Z. mays PGg14 gene for polygalacturonase. | |
| 700354575H1 | g288373 | 86 | −31 | gb105pln | Z. mays PGg6 gene for polygalacturonase. | |
| 700357174H1 | g2814799 | 8 | 0 | gb105eukp | ZK858.6 | |
| 700353676H1 | g22458 | 97 | −86 | gb105pln | Z. mays pollen specific mRNA C-terminal (clone 4H7). | |
| 700354351H1 | g30821 | 15 | 2 | gb105allp | 471 aa polypeptide (gamma 2) | |
| 700352874H1 | g1749576 | 19 | 1 | gb105eukp | similar to Saccharomyces cerevisiae acetyl-CoA acetyltransferase, SWISS-PROT | |
| | | | | | Accession Number P41338 | |
| 700354741H1 | g2301337 | 39 | −1 | gb105allp | unnamed protein product | |
| 700355414H1 | g967967 | 15 | 10 | gb105pln | Brassica campestris ssp. pekinensis (clone bif11) photosystem II 10 kDa polypeptide | |
| | | | | | mRNA, complete cds. | |
| 700282521H2 | g2760334 | 16 | −0 | gb105eukp | F1N21.5 | |
| 700352683H1 | g599624 | 17 | −11 | gb105pln | A. thaliana mRNA for aconitase (ZAPII). | |
| 700382787H1 | g2262097 | 18 | 8 | gb105pln | Arabidopsis thaliana BAC T19F06 genomic sequence, complete sequence. | |
| 700357143H1 | g288611 | 92 | −86 | gb105pln | Z. mays PG gene for polygalacturonase. | |
| 700356281H1 | g288366 | 84 | −53 | gb105pln | Z. mays PGg14 gene for polygalacturonase. | |
| 700356939H1 | g395078 | 33 | −12 | gb105pln | B. rapa ubiquitin and ribosomal protein mRNA, complete CDS's. | |
| 700382258H1 | g2244965 | 18 | −3 | gb105eukp | unnamed protein product | |
| 700357430H1 | g439273 | 32 | 2 | gb105eukp | blt101; not known; 54 amino acids | |
| 700354926H1 | g1667593 | 19 | −1 | gb105pln | Oryza sativa transmembrane protein mRNA, complete cds. | |
| 700383132H1 | g473360 | 21 | 2 | gb105allp | Phl pI allergen | |
| 700357129H1 | g414702 | 31 | −21 | gb105pln | H. lanatus mRNA for allergen Hol-lI. | |
| 700357178H1 | g414702 | 31 | −20 | gb105pln | H. lanatus mRNA for allergen Hol-lI. | |
| 700356745H1 | g397958 | 45 | −23 | gb105pln | Z. mays mRNA for beta-amylase. | |
| 700357491H1 | g2326873 | 45 | −9 | gb105pln | S. tuberosum mRNA for plastidic ATP/ADP-transporter. | |
| 700353293H1 | g22458 | 78 | −84 | gb105pln | Z. mays pollen specific mRNA C-terminal (clone 4H7). | |
| 700356750H1 | g288378 | 70 | −25 | gb105pln | Z. mays gene PG for polygalacturonase. | |
| 700357469H1 | g288366 | 50 | −4 | gb105pln | Z. mays PGg14 gene for polygalacturonase. | |
| 700353962H1 | g1185555 | 21 | −5 | gb105pln | Zea mays glyceraldehyde-3-phosphate dehydrogenase (gpc4) gene, partial cds. | |
| 700353983H1 | g170784 | 50 | −26 | gb105pln | Wheat ubiquitin carrier protein (UBC1) mRNA, complete cds. | |
| 700356487H1 | g168625 | 98 | −63 | gb105pln | Maize sucrose phosphate synthase mRNA, complete cds. | |
| 700354394H1 | g2624416 | 33 | −19 | gb105pln | Zea mays mRNA for ubiquitin carrier protein UBC7. | |
| 700282420H2 | g2827637 | 11 | 4 | gb105eukp | F10N7.190; putative protein | |
| 700383050H1 | g288062 | 13 | 13 | gb105pln | A. thaliana mRNA for ketol-acid reductoisomerase subunit. | |
| 700356974H1 | g1458245 | 10 | 8 | gb105eukp | F54D11.1 | |
| 700382241H1 | g2191157 | 10 | 16 | gb105pln | Arabidopsis thaliana BAC IG002P16. | |
| 700352694H1 | g22416 | 74 | −66 | gb105pln | Z. mays mRNA for polygalacturonase (clone PG1). | |
| 700353643H1 | g36146 | 8 | 7 | gb105allp | ribosomal protein S12 | |
| 700354276H1 | g1469118 | 11 | 6 | gb105eukp | K08E7.2 | |
| 700382657H1 | g929917 | 35 | −12 | gb105pln | Z. mays ABP1 mRNA for actin-depolymerizing factor. | |
| 700355593H1 | g1246115 | 20 | −8 | gb105pln | major allergen Pha a 1 [Phalaris aquatica = canary grass, pollen, mRNA Partial, | |
| | | | | | 1088 nt]. | |
| 700382687H1 | g458972 | 16 | −3 | gb105eukp | F37C12.4 | |
| 700355589H1 | g1022807 | 30 | −26 | gb105eukp | cellulase | |
| 700354824H1 | g992962 | 23 | −3 | gb105eukp | thioredoxin | |
| 700382640H1 | g1235570 | 29 | −17 | gb105pln | P. anserina su12 gene. | |
| 700382823H1 | g1184775 | 47 | −33 | gb105pln | Zea mays glyceraldehyde-3-phosphate dehydrogenase GAPC4 (gpc4) mRNA, | |
| | | | | | complete cds. | |
| 700382779H1 | g2225880 | 43 | −10 | gb105pln | Solanum tuberosum mRNA for eukaryotic initiation factor 5A3, complete cds. | |
| 700353405H1 | g2266661 | 50 | −27 | gb105pln | Hordeum vulgare mRNA for 14-3-3 protein (Hv1433c). | |
| 700353148H1 | g2351062 | 41 | −14 | gb105pln | Arabidopsis thaliana genomic DNA, chromosome 5, P1 clone: MAH20. | |
| 700353834H1 | g1167835 | 18 | 3 | gb105pln | H. lanatus mRNA for Hol lI protein. | |
| 700354652H1 | g1911581 | 30 | −15 | gb105pln | Cyn d 1 = major allergen {clone 14c1 and CD1} [Cynodon dactylon = Bermuda grass, | |
| | | | | | pollen, mRNA Partial, 759 nt]. | |
| 700382033H1 | g2154717 | 22 | 4 | gb105eukp | Kap alpha protein | |
| 700382663H1 | g170047 | 25 | −14 | gb105eukp | PK6; protein kinase | |
| 700382541H1 | g1142653 | 38 | −14 | gb105allp | alpha NAC/1.9.2. protein | |
| 700353168H1 | g312180 | 27 | −48 | gb105pln | Z. mays GapC4 gene. | |
| 700352802H1 | g2226414 | 9 | 5 | gb105eukp | SPBC31F10.03; hypothetical protein | |
| 700356901H1 | g1495259 | 17 | 6 | gb105allp | orf04 | |
| 700382110H1 | g168313 | 9 | 12 | gb105pln | L. perenne pollen allergen (Lol pI) gene, 3′ end. | |
| 700282511H2 | g643596 | 39 | 8 | gb105pln | Corn mRNA for cysteine proteinase, clone CCP, complete cds. | |
| 700357412H1 | g1171161 | 27 | −9 | gb105eukp | pectate lyase homolog | |
| 700282489H2 | g166548 | 33 | 0 | gb105pln | Avena sativa vacuolar H+-ATPase 16 kDa proteolipid subunit (vatp-P1) mRNA, | |
| | | | | | complete cds. | |
| 700382053H1 | g1181261 | 9 | 5 | gb105allp | dolichyl-phosphate beta-glucosyltransferase | |
| 700282473H2 | g833835 | 27 | −4 | gb105eukp | amygdalin hydrolase isoform AH I precursor | |
| 700355237H1 | g1203832 | 25 | −10 | gb105eukp | beta-D-glucan exohydrolase, isoenzyme ExoII; EC 3.2.1.58 | |
| 700354562H1 | g2668737 | 29 | −2 | gb105pln | Zea mays translation initiation factor 5A (TIF5A) mRNA, complete cds. | |
| 700353236H1 | g2529663 | 10 | −2 | gb105eukp | T30B22.6; putative lysophospholipase | |
| 700382790H1 | g22425 | 68 | −59 | gb105pln | Z. mays gene for polygalacturonase (clone 47). | |
| 700352841H1 | g2736190 | 14 | 5 | gb105allp | (p)ppGpp synthetase | |
| 700356637H1 | g313137 | 32 | −1 | gb105pln | Z. mays ZmPRO1 mRNA for profilin 1. | |
| 700352705H1 | g507823 | 21 | −5 | gb105eukp | A622 | |
| 700355858H1 | g1136119 | 34 | −32 | gb105pln | O. sativa mRNA for alpha-tubulin (clone OSTA-111). | |
| 700353742H1 | g957250 | 22 | −2 | gb105pln | FEY = forever young gene [Arabidopsis thaliana, Genomic/mRNA, 1450 nt]. | |
| 700353888H1 | g2388586 | 28 | −13 | gb105eukp | YUP8H12.27 | |
| 700382512H1 | g535587 | 15 | 3 | gb105pln | Arabidopsis thaliana chaperone protein (atj) mRNA, complete cds. | |
| 700356747H1 | g218001 | 39 | 1 | gb105allp | UDP-glucose pyrophosphorylase precursor | |
| 700355846H1 | g288611 | 74 | −79 | gb105pln | Z. mays PG gene for polygalacturonase. | |
| 700356725H1 | g2351063 | 27 | 9 | gb105pln | Arabidopsis thaliana genomic DNA, chromosome 5, P1 clone: MCL19. | |
| 700354550H1 | g416256 | 27 | 3 | gb105pln | Rice mRNA for H+-ATPase, partial sequence. | |
| 700355069H1 | g20321 | 33 | −1 | gb105pln | Oryza sativa RAc1 mRNA for actin. | |
| 700352602H1 | g203918 | 9 | 0 | gb105allp | dihydrodiol dehydrogenase | |
| 700357409H1 | g736271 | 28 | −17 | gb105pln | O. sativa hsp70 gene for heat shock protein 70. | |
| 700353888H1 | g476069 | 19 | −2 | gb105eukp | YBR0909 RAD16; UV damage repair protein | |
| 700353726H1 | g303855 | 26 | 5 | gb105allp | ribosomal protein L7A | |
| 700382311H1 | g2735236 | 11 | 3 | gb105allp | GlxB1 | |
| 700382742H1 | g473359 | 15 | −5 | gb105pln | P. pratense mRNA for Phl pI allergen. | |
| 700382439H1 | g2814650 | 12 | 6 | gb105eukp | R09B3.3 | |
| 700355707H1 | g22458 | 43 | −50 | gb105pln | Z. mays pollen specific mRNA C-terminal (clone 4H7). | |
| 700355438H1 | g22204 | 12 | 15 | gb105pln | Maize Bz-McC gene for UDPglucose flavonoid glycosyl-transferase. | |
| 700355428H1 | g1911581 | 13 | 14 | gb105pln | Cyn d 1 = major allergen {clone 14c1 and CD1} [Cynodon dactylon = Bermuda grass, | |
| | | | | | pollen, mRNA Partial, 759 nt]. | |
| 700353779H1 | g1835731 | 8 | 7 | gb105allp | photosystem II 10 kDa polypeptide | |
| 700353841H1 | g2656029 | 13 | 16 | gb105pln | Arabidopsis thaliana genomic DNA, chromosome 5, P1 clone: MQB2. | |
| 700356130H1 | g304037 | 8 | 2 | gb105eukp | ENOD8; early nodulin | |
| 700282587H2 | g313141 | 24 | −9 | gb105pln | Z. mays ZmPRO3 mRNA for profilin 3. | |
| 700355887H1 | g1750379 | 32 | −42 | gb105pln | Eucalyptus globulus NADP-isocitrate dehydrogenase (EgICDH) mRNA, complete cds. | |
| 700382175H1 | g1903420 | 8 | 4 | gb105allp | p71 | |
| 700356270H1 | g2463564 | 32 | −24 | gb105pln | Oryza sativa mRNA for squalene synthase, complete cds. | |
| 700383122H1 | g1931647 | 10 | 2 | gb105eukp | T19D16.13; endomembrane protein EMP70 recursor isolog | |
| 700356660H1 | g549983 | 69 | −24 | gb105pln | Pennisetum ciliare possible apospory-associated mRNA clone PSUB C, complete cds. | |
| 700355412H1 | g2505864 | 34 | −19 | gb105pln | Arabidopsis thaliana DNA, 24 kb surrounding PFL locus | |
| 700355159H1 | g22421 | 50 | −57 | gb105pln | Z. mays mRNA for polygalacturonase (clone PG3). | |
| 700354692H1 | g167107 | 59 | −40 | gb105pln | Hordeum vulgare vacuolar ATPase B subunit isoform mRNA, complete cds. | |
| 700353969H1 | g313137 | 27 | −5 | gb105pln | Z. mays ZmPRO1 mRNA for profilin 1. | |
| 700354704H1 | g2992 | 19 | −8 | gb105pln | Neurospora crassa crp-1 mRNA for ribosomal protein homologous to Yeast cyH2 gene | |
| | | | | | encoding L29. | |
| 700382807H1 | g22614 | 48 | −52 | gb105pln | S. vulgare pepC gene for PEP carboxylase. | |
| 700354535H1 | g18210 | 17 | 5 | gb105pln | C. reinhardtii mRNA for ribosomal protein P1. | |
| 700383088H1 | g206453 | 57 | −19 | gb105allp | prostatic steroid-binding protein C3 precursor | |
| 700382870H1 | g1218004 | 22 | −8 | gb105eukp | cell plate formation; SDL5A | |
| 700357412H1 | g19451 | 26 | −8 | gb105eukp | pectate lyase | |
| 700355525H1 | g1009231 | 24 | −5 | gb105pln | Nicotiana tabacum EF-1-alpha-related GTP-binding protein (SUP1) mRNA, | |
| | | | | | complete cds. | |
| 700382902H1 | g1568627 | 9 | 0 | gb105eukp | heat shock protein 110 | |
| 700353038H1 | g809535 | 26 | 8 | gb105pln | B. verrucosa mRNA for BETV4 pollen allergen | |
| 700352765H1 | g311334 | 9 | 6 | gb105pln | S. vulgare ltp1 mRNA. | |
| 700356422H1 | g747914 | 30 | −48 | gb105pln | Z. mays CaM1 mRNA for calmodulin. | |
| 700382412H1 | g22456 | 92 | −16 | gb105pln | Z. mays pollen specific mRNA C-terminal (clone 3C12). | |
| 700382094H1 | g20359 | 43 | −73 | gb105pln | Rice gene for 17S ribosomal RNA. | |
| 700352841H1 | g2635224 | 12 | 6 | gb105allp | GTP pyrophosphokinase | |
| 700383115H1 | g872315 | 27 | −10 | gb105allp | 40S ribosomal protein S12 | |
| 700382391H1 | g780371 | 63 | −48 | gb105pln | Oryza sativa enolase mRNA, complete cds. | |
| 700353059H1 | g2245131 | 15 | −7 | gb105eukp | hypothetical protein | |
| 700357196H1 | g2739383 | 20 | −6 | gb105eukp | T9J22.26 | |
| 700356668H1 | g310932 | 41 | −2 | gb105pln | Nicotiana tabacum ribosomal protein L17 mRNA, complete cds. | |
| 700355662H1 | g998429 | 24 | 4 | gb105pln | GRF1 = general regulatory factor [Zea mays, XL80, Genomic, 5348 nt]. | |
| 700282511H2 | g559531 | 92 | −28 | gb105pln | Z. mays mRNA for cysteine proteinase. | |
| 700353874H1 | g313137 | 52 | −66 | gb105pln | Z. mays ZmPRO1 mRNA for profilin 1. | |
| 700352824H1 | g2654088 | 13 | 6 | gb105allp | potassium transporter | |
| 700352770H1 | g2459421 | 31 | −13 | gb105eukp | F4P9.15; similar to calcium-binding EF-hand protein | |
| 700353516H1 | g2384676 | 33 | −13 | gb105pln | Arabidopsis thaliana putative potassium transporter AtKT5p (AtKT5) mRNA, | |
| | | | | | partial cds. | |
| 700354367H1 | g20061 | 17 | −29 | gb105pln | O. hookeri mRNA for protein kinase C inhibitor homologue. | |
| 700353233H1 | g22427 | 54 | −53 | gb105pln | Z. mays gene for polygalacturonase (clone 65). | |
| 700357143H1 | g288378 | 76 | −72 | gb105pln | Z. mays gene PG for polygalacturonase. | |
| 700355995H1 | g1616658 | 53 | −43 | gb105pln | Triticum aestivum adenylosuccinate synthetase mRNA, partial cds. | |
| 700353875H1 | g453975 | 42 | −20 | gb105pln | P. pratense mRNA for profilin-Phl pXI. | |
| 700382242H1 | g871509 | 14 | 12 | gb105pln | P. sativum mRNA for small GTP-binding protein (clone pGTP6). | |
| 700382138H1 | g1542941 | 21 | 8 | gb105eukp | AACT; Acetoacetyl-coenzyme A thiolase; EC 2.3.1.9 | |
| 700356010H1 | g2104535 | 11 | 5 | gb105eukp | T10M13.13; T10M13.13 | |
| 700355742H1 | g1944389 | 9 | 3 | gb105allp | Ray | |
| 700355324H1 | g529705 | 20 | 7 | gb105eukp | cct-6; C. elegans cct-6 (similar to T complex proteins, Tcp-1-related, Cctz) | |
| 700382489H1 | g2344908 | 51 | −41 | gb105allp | IgM heavy chain VH1 region precursor | |
| 700353294H1 | g2408091 | 10 | 2 | gb105eukp | SPAC8C9.08; 40s ribosomal protein | |
| 700353658H1 | g287810 | 60 | −54 | gb105pln | M. sativa mRNA for phosphoprotein phosphatase type 2A. | |
| 700355337H1 | g902583 | 69 | −70 | gb105pln | Zea mays clone MubG1 ubiquitin gene, complete cds. | |
| 700355306H1 | g414907 | 16 | −34 | gb105pln | P. pratense mRNA for pollen allergen PhlpI. | |
| 700354768H1 | g414702 | 31 | −12 | gb105pln | H. lanatus mRNA for allergen Hol-lI. | |
| 700383137H1 | g2851858 | 19 | 4 | gb105allp | unnamed protein product | |
| 700357468H1 | g2276465 | 55 | 4 | gb105eukp | FDH1; NAD-dependent formate dehyddrogenase; EC 1.2.1.2 | |
| 700353136H1 | g2558891 | 21 | 1 | gb105allp | GPI transamidase | |
| 700354280H1 | g22427 | 72 | −58 | gb105pln | Z. mays gene for polygalacturonase (clone 65). | |
| 700356739H1 | g22425 | 26 | −10 | gb105pln | Z. mays gene for polygalacturonase (clone 47). | |
| 700355061H1 | g432606 | 30 | −0 | gb105pln | ric1 = ras-related GTP binding protein possessing GTPase activity [Oryza sativa = rice, | |
| | | | | | Yamahoushi, callus, mRNA, 955 nt]. | |
| 700353266H1 | g402903 | 50 | −45 | gb105pln | Arabidopsis thaliana Columbia laminin receptor-like protein mRNA, complete cds. | |
| 700352918H1 | g473360 | 37 | −1 | gb105eukp | phl pI; Phl pI allergen | |
| 700353657H1 | g287829 | 38 | −49 | gb105pln | Z. mays gene for polygalacturonase. | |
| 700382992H1 | g643596 | 23 | −11 | gb105pln | Corn mRNA for cysteine proteinase, clone CCP, complete cds. | |
| 700352795H1 | g303854 | 52 | −43 | gb105pln | Rice mRNA for ribosomal protein L7A, complete cds. | |
| 700354725H1 | g2345149 | 52 | −44 | gb105pln | Arabidopsis thaliana developmentally regulated GTP binding protein (AtDRG1) mRNA, | |
| | | | | | complete cds. | |
| 700356533H1 | g2570402 | 7 | 6 | gb105allp | EEA1 | |
| 700383182H1 | g1296808 | 48 | −34 | gb105pln | H. vulgare mRNA for xylose isomerase. | |
| 700353418H1 | g288378 | 96 | −43 | gb105pln | Z. mays gene PG for polygalacturonase. | |
| 700353835H1 | g1403043 | 29 | −11 | gb105pln | H. chilense x T. turgidum conv. durum (Tritordeum) mRNA for S-adenosylmethionine | |
| | | | | | decarboxylase. | |
| 700353981H1 | g22149 | 80 | −61 | gb105pln | Z. mays mRNA for alpha-tubulin 3. | |
| 700354903H1 | g414703 | 32 | −5 | gb105eukp | allergen Hol-lI | |
| 700353534H1 | g1498315 | 37 | 6 | gb105eukp | IAP100 | |
| 700382961H1 | g2662342 | 18 | −30 | gb105pln | Oryza sativa mRNA for EF-1 alpha, complete cds. | |
| 700382336H1 | g433041 | 22 | 14 | gb105pln | Zea mays W-22 clone PREM-1B retroelement PREM-1, partial sequence. | |
| 700354406H1 | g2673846 | 43 | 8 | gb105allp | opal codon coding for selenocysteine | |
| 700354427H1 | g17959 | 15 | −8 | gb105eukp | cypro4 | |
| 700282414H2 | g473359 | 18 | 14 | gb105pln | P. pratense mRNA for Phl pI allergen. | |
| 700382511H1 | g169776 | 39 | −39 | gb105pln | Oryza sativa beta-amylase gene, complete cds. | |
| 700352748H1 | g169930 | 34 | −19 | gb105pln | Glycine max calcium dependent protein kinase mRNA. | |
| 700354231H1 | g1185555 | 41 | −25 | gb105pln | Zea mays glyceraldehyde-3-phosphate dehydrogenase (gpc4) gene, partial cds. | |
| 700383088H1 | g56994 | 57 | −19 | gb105allp | steroid binding protein | |
| 700382932H1 | g1765924 | 24 | 1 | gb105allp | B subunit of propionyl-CoA carboxylase | |
| 700354768H1 | g1246115 | 25 | −10 | gb105pln | major allergen Pha a1 [Phalaris aquatica = canary grass, pollen, mRNA Partial, | |
| 1088 nt]. | | | | | | |
| 700356101H1 | g168403 | 16 | −0 | gb105pln | Maize actin 1 gene (MAc1), complete cds. | |
| 700356990H1 | g22425 | 98 | −38 | gb105pln | Z. mays gene for polygalacturonase (clone 47). | |
| 700354713H1 | g1304265 | 34 | −23 | gb105pln | Wheat mRNA for HALF-1, complete cds. | |
| 700355153H1 | g1944572 | 20 | −24 | gb105pln | H. vulgare partial PAL mRNA for phenylalanine ammonia-lyase (1831 bp). | |
| 700354625H1 | g414703 | 9 | 7 | gb105eukp | allergen Hol-lI | |
| 700354406H1 | g306765 | 43 | 7 | gb105allp | glutathione peroxidase | |
| 700282482H2 | g1001649 | 12 | 1 | gb105allp | DNA gyrase A subunit | |
| 700352614H1 | g300115 | 37 | −5 | gb105allp | CD9 antigen [human, leukocytes, Peptide, 228 aa] | |
| 700354776H1 | g30381 | 10 | −1 | gb105allp | cytochrome oxidase subunit VIb | |
| 700352870H1 | g414907 | 48 | −35 | gb105pln | P. pratense mRNA for pollen allergen PhlpI. | |
| 700282457H2 | g1718236 | 8 | −1 | gb105eukp | (1,4)-beta-xylan endohydrolase, isoenzyme X-I; EC 3.2.1.8 | |
| 700382206H1 | g2501849 | 42 | −24 | gb105pln | Nicotiana tabacum GDP dissociation inhibitor (GDI) mRNA, complete cds. | |
| 700354748H1 | g487923 | 23 | 4 | gb105pln | Wheat mRNA for wpk4 protein kinase, complete cds. | |
| 700382072H1 | g2673869 | 24 | −6 | gb105pln | Antirrhinum majus mRNA for fimbriata-associated protein 2, partial. | |
| 700355985H1 | g1519248 | 42 | −24 | gb105pln | Oryza sativa GF14-b protein mRNA, complete cds. | |
| 700355846H1 | g288378 | 59 | −60 | gb105pln | Z. mays gene PG for polygalacturonase. | |
| 700356002H1 | g2340821 | 14 | −4 | gb105eukp | oxi-1; E6-AP ubiquitin-protein ligase | |
| 700352672H1 | g1930152 | 16 | 7 | gb105pln | Aspergillus fumigatus heat shock protein (Hsp1) mRNA, partial cds. | |
| 700354316H1 | g237969 | 4 | 5 | gb105eukp | nitrate reductase heme domain, NR; nitrate reductase heme domain | |
| 700357492H1 | g2668745 | 58 | −49 | gb105pln | Zea mays inorganic pyrophosphatase (IPP) mRNA, complete cds. | |
| 700356144H1 | g288611 | 49 | −76 | gb105pln | Z. mays PG gene for polygalacturonase. | |
| 700382267H1 | g606751 | 9 | 8 | gb105eukp | Rox2; Single-strand nucleic acid binding protein; RNA binding protein | |
| 700352957H1 | g2827142 | 32 | −19 | gb105pln | Arabidopsis thaliana cellulose synthase catalytic subunit (Ath-B) mRNA, complete cds. | |
| 700352771H1 | g218112 | 48 | −57 | gb105pln | Rice mRNA for ribosomal protein L41 (340 gene), partial sequence. | |
| 700356911H1 | g22425 | 96 | −36 | gb105pln | Z. mays gene for polygalacturonase (clone 47). | |
| 700354675H1 | g11288 | 21 | −3 | gb105eukp | ribosomal protein L5 (AA 1-181) | |
| 700282587H2 | g313137 | 40 | −60 | gb105pln | Z. mays ZmPRO1 mRNA for profilin 1. | |
| 700353995H1 | g288366 | 78 | −6 | gb105pln | Z. mays PGg14 gene for polygalacturonase. | |
| 700356487H1 | g1311478 | 60 | −36 | gb105pln | Rice sucrose phosphate synthase (SPS) gene, complete cds. | |
| 700352641H1 | g435312 | 16 | −4 | gb105pln | Z. mays mRNA for beta-D-glucosidase. | |
| 700382175H1 | g2062605 | 8 | 3 | gb105allp | brain secretory protein hSec10p | |
| 700356775H1 | g289124 | 46 | −9 | gb105pln | Medicago sativa MAP kinase MsERK1 mRNA, complete cds. | |
| 700355463H1 | g22416 | 33 | −61 | gb105pln | Z. mays mRNA for polygalacturonase (clone PG1). | |
| 700357129H1 | g414907 | 31 | −22 | gb105pln | P. pratense mRNA for pollen allergen PhlpI. | |
| 700357178H1 | g414907 | 31 | −21 | gb105pln | P. pratense mRNA for pollen allergen PhlpI. | |
| 700356950H1 | g1213072 | 52 | −2 | gb105pln | O. sativa (Tainong 67) mRNA for heat shock protein 17.8. | |
| 700355186H1 | g255568 | 9 | 16 | gb105pln | pollen specific protein [Zea mays = corn, mRNA, 943 nt]. | |
| 700356164H1 | g288378 | 54 | −60 | gb105pln | Z. mays gene PG for polygalacturonase. | |
| 700353026H1 | g1002533 | 44 | −0 | gb105eukp | ACT11; actin-11 | |
| 700383090H1 | g1750404 | 36 | −10 | gb105eukp | B0412.4 | |
| 700352614H1 | g34769 | 37 | −5 | gb105allp | MRP-1 (motility related protein) | |
| 700382108H1 | g510274 | 11 | 11 | gb105pln | Arabidopsis thaliana DET1 gene, complete cds. | |
| 700352766H1 | g488576 | 25 | −6 | gb105pln | Medicago sativa Regen S clone pH3c131 histone H3.2 mRNA, complete cds. | |
| 700382662H1 | g255568 | 28 | −23 | gb105pln | pollen specific protein [Zea mays = corn, mRNA, 943 nt]. | |
| 700353266H1 | g16379 | 50 | −45 | gb105pln | A. thaliana mRNA for laminin receptor homologue. | |
| 700356675H1 | g1399270 | 35 | −1 | gb105pln | Arabidopsis thaliana calmodulin-domain protein kinase CDPK isoform 2 (CPK2) | |
| | | | | | mRNA, complete cds. | |
| 700353695H1 | g168315 | 17 | 2 | gb105pln | L. perenne pollen allergen (Lol pI) gene, complete cds. | |
| 700353609H1 | g1707372 | 28 | 0 | gb105eukp | smt3; ubiquitin-like protein | |
| 700354786H1 | g168313 | 13 | 2 | gb105pln | L. perenne pollen allergen (Lol pI) gene, 3′ end. | |
| 700382351H1 | g533692 | 16 | −0 | gb105eukp | protease inhibitor | |
| 700382721H1 | g499011 | 69 | −59 | gb105pln | S. vulgare SoAc1 mRNA. | |
| 700382295H1 | g537274 | 27 | −27 | gb105pln | Histoplasma capsulatum elongation factor 1-alpha (Hctef1) mRNA, complete cds. | |
| 700356821H1 | g1498387 | 41 | −55 | gb105pln | Zea mays actin (Maz83) gene, partial cds. | |
| 700354175H1 | g886402 | 35 | −25 | gb105pln | Oryza sativa MADS-box protrein (mADS4) mRNA, complete cds. | |
| 700382257H1 | g2656031 | 18 | −25 | gb105pln | Arabidopsis thaliana genomic DNA, chromosome 5, P1 clone: MXC20. | |
| 700352852H1 | g22425 | 89 | −75 | gb105pln | Z. mays gene for polygalacturonase (clone 47). | |
| 700356575H1 | g2415701 | 18 | −21 | gb105pln | P. pratense mRNA for profilin 4. | |
| 700353153H1 | g602252 | 60 | −53 | gb105pln | Zea mays enolase (eno2) mRNA, complete cds. | |
| 700354260H1 | g1145627 | 19 | −9 | gb105eukp | lipase | |
| 700357125H1 | g22458 | 70 | −85 | gb105pln | Z. mays pollen speicif mRNA C-terminal (clone 4H7). | |
| 700352712H1 | g168460 | 49 | −46 | gb105pln | Zea mays cyclophilin (CyP) mRNA, complete cds. | |
| 700354455H1 | g2088650 | 11 | 7 | gb105eukp | T28M21.13; peroxisomal ATP/ADP carrier protein isolog | |
| 700357180H1 | g2388580 | 5 | 6 | gb105eukp | YUP8H12.22 | |
| 700353245H1 | g20309 | 22 | 2 | gb105pln | O. sativa DNA for pollen-specific gene. | |
| 700353542H1 | g2832623 | 33 | −16 | gb105eukp | F13C5.120; protein kinase-like protein | |
| 700355414H1 | g16446 | 10 | 9 | gb105pln | A. thaliana gene for photosystem II 10 kDa polypeptide. | |
| 700353258H1 | g1911581 | 26 | −9 | gb105pln | Cyn d 1 = major allergen {clone 14c1 and CD1} [Cynodon dactylon = Bermuda grass, | |
| | | | | | pollen, mRNA Partial, 759 nt]. | |
| 700353796H1 | g1419875 | 10 | 7 | gb105eukp | ORF YOL060c | |
| 700355783H1 | g407488 | 11 | 6 | gb105eukp | unknown | |
| 700356594H1 | g21629 | 19 | 17 | gb105pln | Sorghum vulgare mRNA for phosphoenolpyruvate carboxylase (PEPC). | |
| 700352639H1 | g2398678 | 33 | −8 | gb105pln | Morinda citrifolia mRNA for 3-deoxy-D-arabino-heptulosonate 7-phosphate synthase, | |
| | | | | | DS1. | |
| 700355701H1 | g22416 | 41 | −11 | gb105pln | Z. mays mRNA for polygalacturonase (clone PG1). | |
| 700383138H1 | g450367 | 30 | −15 | gb105pln | T. aestivum mRNA for eukaryotic initiation factor 4f subunit p26. | |
| 700357174H1 | g2435604 | 10 | −1 | gb105eukp | F08F1.7 | |
| 700382763H1 | g1621033 | 94 | −45 | gb105allp | adducin gamma subunit | |
| 700353030H1 | g473359 | 39 | −2 | gb105pln | P. pratense mRNA for Phl pI allergen. | |
| 700354828H1 | g22421 | 50 | −52 | gb105pln | Z. mays mRNA for polygalacturonase (clone PG3). | |
| 700352763H1 | g2154727 | 44 | −41 | gb105pln | C. dactylon mRNA for profilin 2. | |
| 700352909H1 | g22592 | 45 | −20 | gb105pln | S. vulgare PEPC gene for phosphoenolpyruvate carboxylase. | |
| 700352974H1 | g2352555 | 77 | −6 | gb105eukp | Hsp82; 82 kDa heat shock protein | |
| 700382280H1 | g2505870 | 27 | −24 | gb105eukp | hypothetical protein | |
| 700354212H1 | g2668737 | 45 | 6 | gb105pln | Zea mays translation initiation factor 5A (TIF5A) mRNA, complete cds. | |
| 700355461H1 | g2282583 | 82 | −69 | gb105pln | Zea mays elongation factor 1-alpha (EF1-A) mRNA, complete cds. | |
| 700355949H1 | g2274990 | 22 | −10 | gb105pln | Hordeum vulgare mRNA for expressed sequence tag. | |
| 700356976H1 | g1147612 | 54 | −3 | gb105eukp | MOT1; Mot1p: a putative helicase | |
| 700352677H1 | g561663 | 48 | −34 | gb105pln | Rice mRNA, partial homologous to ribosomal protein coding sequence. | |
| 700356111H1 | g829147 | 40 | −80 | gb105pln | Z. mays gene for cyclophilin. | |
| 700383130H1 | g22223 | 52 | −47 | gb105pln | Maize cab-1 gene for chlorophyll a/b-binding protein. | |
| 700353278H1 | g1167835 | 25 | −6 | gb105pln | H. lanatus mRNA for Hol lI protein. | |
| 700355543H1 | g1620022 | 26 | −6 | gb105allp | ribosomal protein L14 | |
| 700352653H1 | g22458 | 100 | −60 | gb105pln | Z. mays pollen specific mRNA C-terminal (clone 4H7). | |
| 700356516H1 | g2302294 | 20 | −3 | gb105allp | unnamed protein product | |
| 700353987H1 | g22420 | 53 | 6 | gb105pln | Z. mays mRNA for polygalacturonase (clone PG2). | |
| 700353680H1 | g1184773 | 79 | −76 | gb105pln | Zea mays glyceraldehyde-3-phosphate dehydrogenase GAPC3 (gpc3) mRNA, | |
| | | | | | complete cds. | |
| 700354394H1 | g170783 | 18 | 1 | gb105pln | T. aestivum ubiquitin carrier protein mRNA. | |
| 700354267H1 | g1771781 | 27 | −4 | gb105pln | T. aestivum mRNA for beta-amylase. | |
| 700355324H1 | g2300244 | 25 | 6 | gb105allp | unnamed protein product | |
| 700382310H1 | g293901 | 39 | −22 | gb105pln | Zea mays Zea mI gene, complete cds. | |
| 700382074H1 | g312180 | 34 | −75 | gb105pln | Z. mays GapC4 gene. | |
| 700357443H1 | g2827551 | 21 | −10 | gb105eukp | T12H17.130; predicted protein | |
| 700352718H1 | g436782 | 22 | −4 | gb105pln | Rice mRNA for cyc07, complete cds. | |
| 700355323H1 | g168313 | 23 | 9 | gb105pln | L. perenne pollen allergen (Lol pI) gene, 3′ end. | |
| 700382027H1 | g1495248 | 41 | −35 | gb105pln | A. thaliana mRNA for unknown protein, eRF1-3 gene. | |
| 700355006H1 | g20321 | 23 | −7 | gb105pln | Oryza sativa RAc1 mRNA for actin. | |
| 700282489H2 | g1622720 | 25 | 9 | gb105pln | Kalanchoe daigremontiana V-type H+-ATPase 16 kDa subunit mRNA, complete cds. | |
| 700353991H1 | g397958 | 40 | −48 | gb105pln | Z. mays mRNA for beta-amylase. | |
| 700357426H1 | g2182652 | 6 | 3 | gb105allp | Y4uB | |
| 700352957H1 | g2827138 | 37 | −26 | gb105pln | Arabidopsis thaliana cellulose synthase catalytic subunit (RSW1) gene, complete cds. | |
| 700355216H1 | g313137 | 98 | −92 | gb105pln | Z. mays ZmPRO1 mRNA for profilin 1. | |
| 700352941H1 | g2081611 | 47 | −14 | gb105pln | Rice mRNA for deltal-pyrroline-5-carboxylate synthetase, complete cds. | |
| 700353102H1 | g288366 | 95 | −59 | gb105pln | Z. mays PGg14 gene for polygalacturonase. | |
| 700356645H1 | g1173556 | 43 | −5 | gb105pln | Oryza sativa Ory s 1 mRNA, complete cds. | |
| 700354803H1 | g1632830 | 22 | −3 | gb105pln | R. communis mRNA (unknown). | |
| 700355223H1 | g435174 | 43 | −51 | gb105pln | A. sativa (Pewi) ASTCP-K36 mRNA for t complex polypeptide 1. | |
| 700382074H1 | g1184775 | 40 | −61 | gb105pln | Zea mays glyceraldehyde-3-phosphate dehydrogenase GAPC4 (gpc4) mRNA, | |
| | | | | | complete cds. | |
| 700382150H1 | g790583 | 25 | −10 | gb105eukp | ATOZI1 | |
| 700352972H1 | g1815759 | 26 | 3 | gb105eukp | pollen-specific protein | |
| 700353883H1 | g16836 | 20 | 2 | gb105allp | probable protease inhibitor P322 precursor (Bowman-Birk) | |
| 700353802H1 | g928932 | 14 | −6 | gb105eukp | D18; sugar metabolism; homolog of dTDP-glucose 4-6-dehydratases | |
| 700356069H1 | g1370204 | 62 | 14 | gb105pln | L. japonicus mRNA for small GTP-binding protein, RAN1B. | |
| 700352683H1 | g868002 | 22 | −17 | gb105pln | Pumpkin mRNA for aconitase, complete cds. | |
| 700354623H1 | g2264305 | 18 | 6 | gb105pln | Arabidopsis thaliana genomic DNA, chromosome 5, P1 clone: MBK23, complete | |
| | | | | | sequence. | |
| 700282418H2 | g1255713 | 82 | −80 | gb105pln | Sorghum bicolor granule-bound starch synthase precursor (Wx) mRNA, nuclear gene | |
| | | | | | encoding chloroplast protein, complete cds. | |
| 700352841H1 | g466814 | 14 | 6 | gb105allp | spoT; guanosine-3′,5′bis(diphosphate)3′-pyrophosphohydrolase; B1177_C1_168 | |
| 700352834H1 | g2342735 | 21 | −11 | gb105eukp | T14G11.28 | |
| 700352911H1 | g544501 | 10 | 2 | gb105eukp | SEC13; Sec13p | |
| 700354342H1 | g717059 | 11 | 14 | gb105pln | Yeast (Saccharomyces cerevisiae) chromosome XII cosmid 9324. | |
| 700282491H2 | g1280409 | 36 | −15 | gb105pln | Tacca plantaginea (Hance) Drenth 18S ribosomal RNA (18S rRNA) gene, | |
| | | | | | segment 1 of 2. | |
| 700382986H1 | g473360 | 8 | 8 | gb105eukp | phl pI; Phl pI allergen | |
| 700355540H1 | g1296435 | 16 | −2 | gb105eukp | leg10; legumin-like protein | |
| 700356573H1 | g2738247 | 42 | −31 | gb105pln | Arabidopsis thaliana cobalamin-independent methionine synthase (ATCIMS) mRNA, | |
| | | | | | complete cds. | |
| 700356660H1 | g531481 | 69 | −24 | gb105pln | P. ciliare (Higgins) apospory associated mRNA, 1398 bp. | |
| 700355901H1 | g1155262 | 22 | −3 | gb105pln | Arabidopsis thaliana eukaryotic release factor 1 homolog (eRF1) mRNA, partial cds. | |
| 700356144H1 | g288378 | 35 | −56 | gb105pln | Z. mays gene PG for polygalacturonase. | |
| 700356637H1 | g453975 | 31 | 11 | gb105pln | P. pratense mRNA for profilin-Phl pXI. | |
| 700355533H1 | g2583106 | 29 | −20 | gb105pln | Arabidopsis thaliana chromosome II BAC F4L23 genomic sequence, complete sequence. | |
| 700352705H1 | g2687724 | 20 | −4 | gb105eukp | NADPH:isoflavone reductase | |
| 700352676H1 | g1622720 | 34 | −14 | gb105pln | Kalanchoe daigremontiana V-type H+-ATPase 16 kDa subunit mRNA, complete cds. | |
| 700356746H1 | g414908 | 27 | 7 | gb105eukp | pollen allergen Phl pI | |
| 700282508H2 | g833835 | 8 | −6 | gb105eukp | amygdalin hydrolase isoform AH I precursor | |
| 700356415H1 | g168030 | 18 | 15 | gb105pln | Aspergillus nidulans calmodulin gene, complete cds. | |
| 700382357H1 | g623585 | 24 | −0 | gb105pln | Nicotiana tabacum SR1 Nt-rab6 mRNA, complete cds. | |
| 700354894H1 | g22416 | 60 | −68 | gb105pln | Z. mays mRNA for polygalacturonase (clone PG1). | |
| 700353191H1 | g246 | 10 | 2 | gb105allp | NADH dehydrogenase | |
| 700354768H1 | g414907 | 30 | −11 | gb105pln | P. pratense mRNA for pollen allergen PhlpI. | |
| 700356809H1 | g22144 | 52 | −18 | gb105pln | Maize anaerobically regulated gene for fructose bisphosphate aldolase (EC 4.1.2.13). | |
| 700356493H1 | g1694620 | 35 | −14 | gb105pln | Cucurbita sp. mRNA for 3-ketoacyl-CoA thiolase, complete cds. | |
| 700352658H1 | g287829 | 98 | −58 | gb105pln | Z. mays gene for polygalacturonase. | |
| 700352812H1 | g596077 | 24 | −55 | gb105pln | Zea mays thiamine biosynthetic enzyme (thi1-1) mRNA, complete cds. | |
| 700354886H1 | g1209258 | 12 | 5 | gb105allp | protease inhibitor II | |
| 700355061H1 | g1616613 | 28 | 2 | gb105pln | N. plumbaginifolia mRNA for small GTP-binding protein, clone Np43GTP. | |
| 700356055H1 | g22425 | 49 | −62 | gb105pln | Z. mays gene for polygalacturonase (clone 47). | |
| 700357422H1 | g2511715 | 27 | −8 | gb105eukp | putative phosphatidylinositol-4-phosphate 5-kinase | |
| 700383122H1 | g2435604 | 7 | 5 | gb105eukp | F08F1.7 | |
| 700357441H1 | g1167835 | 23 | −4 | gb105pln | H. lanatus mRNA for Hol lI protein. | |
| 700354757H1 | g1067106 | 23 | 3 | gb105allp | ZK757.3 | |
| 700355876H1 | g2384757 | 34 | −24 | gb105pln | Oryza sativa GDP dissociation inhibitor protein OsGDI1 (OsGDI1) mRNA, | |
| | | | | | complete cds. | |
| 700382268H1 | g510238 | 4 | 8 | gb105eukp | OPT1-1; oligopeptide transporter 1-1 | |
| 700352955H1 | g432359 | 78 | −20 | gb105allp | ribosomal protein | |
| 700354757H1 | g2149640 | 35 | 5 | gb105eukp | AGO1; leaf development; Argonaute protein | |
| 700353060H1 | g1045304 | 81 | −73 | gb105pln | Zea mays acetyl-coenzyme A carboxylase mRNA, complete cds. | |
| 700355485H1 | g2213594 | 31 | −15 | gb105eukp | T7N9.14 | |
| 700282455H2 | g22416 | 53 | −79 | gb105pln | Z. mays mRNA for polygalacturonase (clone PG1). | |
| 700357153H1 | g293886 | 42 | −80 | gb105pln | Zea mays glyceraldehyde-3-phosphate dehydrogenase mRNA, 3′ end, (clone GAPC3). | |
| 700356279H1 | g2565339 | 31 | −17 | gb105pln | Lupinus luteus ribosomal protein S14 (rps14) mRNA, complete cds. | |
| 700282508H2 | g21953 | 8 | −6 | gb105eukp | Li; beta-glucosidase; EC 3.2.1.21; linamarase | |
| 700352858H1 | g1931636 | 20 | −26 | gb105pln | Arabidopsis thaliana BAC T19D16 genomic sequence. | |
| 700353511H1 | g1817577 | 63 | −36 | gb105allp | alpha-globin 1 | |
| 700355824H1 | g218168 | 35 | −67 | gb105pln | Rice mRNA for succinate dehydrogenase (K7 gene), partial sequence. | |
| 700355983H1 | g2443751 | 15 | 5 | gb105eukp | FUM1; fumarase; EC 4.2.1.2 | |
| 700354903H1 | g414908 | 32 | −5 | gb105eukp | pollen allergen Phl pI | |
| 700355856H1 | g1633049 | 10 | 4 | gb105eukp | RpL22; ribosomal protein Rp122 | |
| 700282518H2 | g1518539 | 42 | −20 | gb105pln | Glycine max UDP-glucose dehydrogenase mRNA, complete cds. | |
| 700353564H1 | g2760167 | 25 | −10 | gb105pln | Arabidopsis thaliana genomic DNA, chromosome 5, P1 clone: MCO15, complete | |
| | | | | | sequence. | |
| 700354503H1 | g603866 | 26 | −3 | gb105pln | N. tabacum mRNA for L-Glutamate:tRNA-Glu ligase. | |
| 700355883H1 | g22421 | 97 | −91 | gb105pln | Z. mays mRNA for polygalacturonase (clone PG3). | |
| 700282464H1 | g288612 | 13 | 1 | gb105allp | polygalacturonase | |
| 700382290H1 | g758701 | 30 | −0 | gb105eukp | Ch FatB1; 16:0-ACP thioesterase preprotein | |
| 700354917H1 | g288373 | 98 | −63 | gb105pln | Z. mays PGg6 gene for polygalacturonase. | |
| 700353019H1 | g17561 | 16 | 8 | gb105allp | PROTEINASE INHIBITOR | |
| 700354351H1 | g165011 | 17 | 2 | gb105allp | eucaryotic release factor (eRF) | |
| 700382117H1 | g2809246 | 39 | −21 | gb105eukp | F24O1.15 | |
| 700352644H1 | g20321 | 59 | −63 | gb105pln | Oryza sativa RAc1 mRNA for actin. | |
| 700353834H1 | g473359 | 15 | 7 | gb105pln | P. pratense mRNA for Phl pI allergen. | |
| 700355537H1 | g1449178 | 28 | −15 | gb105pln | Nicotiana tabacum mRNA for N-ethylmaleimide sensitive fusion protein, complete cds. | |
| 700357171H1 | g288611 | 21 | −17 | gb105pln | Z. mays PG gene for polygalacturonase. | |
| 700382134H1 | g1255954 | 21 | −11 | gb105eukp | thioredoxin | |
| 700354991H1 | g807577 | 36 | 2 | gb105eukp | GTP-binding protein | |
| 700382110H1 | g414702 | 9 | 5 | gb105pln | H. lanatus mRNA for allergen Hol-lI. | |
| 700282591H2 | g1292952 | 23 | 5 | gb105allp | 3-hydroxy-3-methylglutaryl-CoA lyase | |
| 700382002H1 | g168460 | 49 | −68 | gb105pln | Zea mays cyclophilin (CyP) mRNA, complete cds. | |
| 700352725H1 | g1532210 | 22 | −29 | gb105eukp | cypA; cyclophilin A | |
| 700354747H1 | g169127 | 17 | −13 | gb105pln | Pisum sativum (clone pCLp) nuclear encoded precursor to chloroplast protein mRNA, | |
| | | | | | complete cds. | |
| 700282513H2 | g1653665 | 19 | −17 | gb105allp | peptide chain release factor | |
| 700383006H1 | g393400 | 47 | −45 | gb105pln | Z. mays mRNA for alpha-tubulin. | |
| 700352783H1 | g1184165 | 24 | −6 | gb105eukp | knolle | |
| 700353547H1 | g2342684 | 35 | −10 | gb105eukp | F7G19.14; F7G19.14 | |
| 700352989H1 | g288373 | 72 | −56 | gb105pln | Z. mays PGg6 gene for polygalacturonase. | |
| 700353874H1 | g453975 | 40 | −20 | gb105pln | P. pratense mRNA for profilin-Phl pXI. | |
| 700355945H1 | g168460 | 71 | −76 | gb105pln | Zea mays cyclophilin (CyP) mRNA, complete cds. | |
| 700355459H1 | g974781 | 55 | −43 | gb105pln | C. blumei kinetoplast met gene for cobalamine-independent methionine synthase. | |
| 700383039H1 | g169802 | 24 | 1 | gb105pln | Rice oryzacystatin-II mRNA, complete cds. | |
| 700355947H1 | g415896 | 8 | 6 | gb105eukp | PHL PII, pollen allergen | |
| 700353922H1 | g21598 | 48 | −36 | gb105pln | S. tuberosum mRNA for UDP-glucose pyrophosphorylase. | |
| 700356485H1 | g248336 | 73 | −42 | gb105pln | polyubiquitin [maize, Genomic, 3841 nt]. | |
| 700382004H1 | g303852 | 25 | −46 | gb105pln | Rice mRNA for ribosomal protein L3, complete cds. | |
| 700282527H2 | g19495 | 20 | 9 | gb105pln | L. perenne RNA for Lol pI gene. | |
| 700282541H2 | g1627870 | 13 | 7 | gb105eukp | F30A10.6 | |
| 700353610H1 | g20237 | 18 | −21 | gb105pln | O. sativa (rice) constitutive GOS2 gene. | |
| 700282482H2 | g1019146 | 8 | 6 | gb105allp | DNA gyrase | |
| 700356422H1 | g1292852 | 25 | −3 | gb105pln | F. sylvatica mRNA for CaMF protein. | |
| 700354374H1 | g2443755 | 23 | −10 | gb105eukp | CYP5; cyclophilin | |
| 700355762H1 | g17789 | 29 | −9 | gb105eukp | Bp10 | |
| 700355583H1 | g575291 | 32 | −25 | gb105pln | H. vulgare mRNA for SNF1-related protein kinase. | |
| 700356834H1 | g22427 | 80 | −84 | gb105pln | Z. mays gene for polygalacturonase (clone 65). | |
| 700383168H1 | g22458 | 43 | −75 | gb105pln | Z. mays pollen specific mRNA C-terminal (clone 4H7). | |
| 700356670H1 | g2695945 | 29 | −5 | gb105eukp | H(+)-transporting ATPase-like protein | |
| 700382544H1 | g2252869 | 29 | −4 | gb105eukp | AT0ZI1 | |
| 700355562H1 | g2827001 | 65 | −60 | gb105pln | Triticum aestivum 70 kDa heat shock protein (TaHSP70d) mRNA, complete cds. | |
| 700356112H1 | g886402 | 75 | −68 | gb105pln | Oryza sativa MADS-box protein (MADS4) mRNA, complete cds. | |
| 700353027H1 | g1519248 | 49 | −17 | gb105pln | Oryza sativa GF14-b protein mRNA, complete cds. | |
| 700353141H1 | g1655479 | 21 | 0 | gb105pln | Arabidopsis thaliana mRNA for gamma subunit of mitochondrial F1-ATPase, | |
| | | | | | complete cds. | |
| 700382871H1 | g1895083 | 46 | −56 | gb105pln | Zea mays golgi associated protein se-wap41 mRNA, complete cds. | |
| 700353875H1 | g313139 | 47 | −23 | gb105pln | Z. mays ZmPRO2 mRNA for profilin 2. | |
| 700355627H1 | g899607 | 15 | −2 | gb105pln | Zea mays polyubiquitin (MubC5) mRNA, complete cds. | |
| 700357187H1 | g414907 | 31 | −22 | gb105pln | P. pratense mRNA for pollen allergen PhlpI. | |
| 700353986H1 | g16509 | 30 | −11 | gb105pln | A. thaliana mRNA for succinate-CoA ligase (GCP-forming). | |
| 700383178H1 | g1008915 | 60 | −30 | gb105allp | proteasome activator hPA28 suunit beta | |
| 700352941H1 | g1480669 | 37 | −6 | gb105pln | Solanum lycopersicum delta 1-pyrroline-5-carboxylate synthetase (tomPRO2) mRNA, | |
| | | | | | complete cds. | |
| 700355915H1 | g288373 | 98 | −86 | gb105pln | Z. mays PGg6 gene for polygalacturonase. | |
| 700382643H1 | g1899187 | 25 | −12 | gb105pln | Nicotiana tabacum DNA binding protein ACBF mRNA, complete cds. | |
| 700352775H1 | g288378 | 63 | −63 | gb105pln | Z. mays gene PG for polygalacturonase. | |
| 700355812H1 | g22416 | 71 | −84 | gb105pln | Z. mays mRNA for polygalacturonase (clone PG1). | |
| 700356445H1 | g168313 | 22 | 6 | gb105pln | L. perenne pollen allergen (Lol pI) gene, 3′ end. | |
| 700352775H1 | g22427 | 59 | −63 | gb105pln | Z. mays gene for polygalacturonase (clone 65). | |
| 700382611H1 | g1246115 | 22 | −7 | gb105pln | major allergen Pha a 1 [Phalaris aquatica = canary grass, pollen, mRNA Partial, | |
| | | | | | 1088 nt]. | |
| 700382870H1 | g807577 | 22 | −8 | gb105eukp | GTP-binding protein | |
| 700352787H1 | g2351062 | 18 | −2 | gb105pln | Arabidopsis thaliana genomic DNA, chromosome 5, P1 clone: MAH20. | |
| 700355738H1 | g16427 | 11 | 5 | gb105eukp | protease inhibitor II | |
| 700356881H1 | g2828188 | 15 | −8 | gb105pln | Arabidopsis thaliana genomic DNA, chromosome 5, TAC clone: K3I3, complete | |
| | | | | | sequence. | |
| 700356511H1 | g288373 | 48 | −65 | gb105pln | Z. mays PGg6 gene for polygalacturonase. | |
| 700352844H1 | g1296808 | 33 | −32 | gb105pln | H. vulgare mRNA for xylose isomerase. | |
| 700353567H1 | g1532167 | 24 | 3 | gb105eukp | AT.I.24-5 | |
| 700354175H1 | g2529339 | 27 | −5 | gb105pln | Zea maize AGAMOUS homologue mRNA, complete cds. | |
| 700382820H1 | g972368 | 14 | 2 | gb105allp | 60 S ribosomal protein L35 | |
| 700355834H1 | g22458 | 50 | −73 | gb105pln | Z. mays pollen specific mRNA C-terminal (clone 4H7). | |
| 700382013H1 | g940182 | 14 | −2 | gb105allp | tRNA-Guanine Transglycosylase | |
| 700355002H1 | g170775 | 56 | −35 | gb105pln | Wheat translation elongation factor 1 alpha-subunit (TEF1) mRNA, complete cds. | |
| 700353534H1 | g1495768 | 37 | 6 | gb105allp | chloroplast inner envelope protein, 110 kD (IEP110) | |
| 700383157H1 | g1814402 | 34 | −26 | gb105pln | Mesembryanthemum crystallinum methionine synthase (MetE) mRNA, complete cds. | |
| 700383114H1 | g288373 | 84 | −70 | gb105pln | Z. mays PGg6 gene for polygalacturonase. | |
| 700355710H1 | g20163 | 54 | −45 | gb105pln | O. sativa Rr15 mRNA for 5S ribosomal RNA. | |
| 700282473H2 | g1236961 | 31 | −6 | gb105eukp | hydrolysis of the cyangenic glucoside (R)-pruansin; pruansin hydrolase precursor; | |
| | | | | | EC 3.2.1.21 | |
| 700352802H1 | g2827524 | 12 | −2 | gb105eukp | F8F16.110; predicted protein | |
| 700352953H1 | g882247 | 38 | −7 | gb105pln | Urochloa panicoides carbonic anhydrase 1 mRNA, complete cds. | |
| 700355073H1 | g1575322 | 48 | 7 | gb105allp | APS kinase | |
| 700382510H1 | g22456 | 63 | −62 | gb105pln | Z. mays pollen specific mRNA C-terminal (clone 3C12). | |
| 700354757H1 | g1167810 | 23 | 3 | gb105eukp | T22B3.2 | |
| 700355168H1 | g2580496 | 23 | −23 | gb105pln | Papaver somniferum NADPH:ferrihemoprotein oxidoreductase mRNA, complete cds. | |
| 700355760H1 | g22136 | 65 | −44 | gb105pln | Maize Adh2-N mRNA for alcohol dehydrogenase 2. | |
| 700382107H1 | g529357 | 11 | 6 | gb105allp | No definition line found | |
| 700382308H1 | g600766 | 14 | 13 | gb105pln | Oryza sativa cyclophilin 2 (Cyp2) gene, complete cds. | |
| 700352835H1 | g2641637 | 27 | −39 | gb105pln | Arabidopsis thaliana DnaJ homolog AtJ3 (ATJ3) gene, complete cds. | |
| 700353742H1 | g348719 | 26 | −8 | gb105pln | MtNMedicago truncatula protochlorophyllide reductase homologue mRNA, | |
| | | | | | complete cds. | |
| 700382541H1 | g1666690 | 38 | −14 | gb105allp | alpha-NAC, non-muscle form | |
| 700353032H1 | g2073374 | 70 | −34 | gb105pln | Rice mRNA for farnesyl pyrophosphate synthase, complete cds. | |
| 700355629H1 | g20323 | 30 | −22 | gb105pln | O. sativa RAc1 gene for actin. | |
| 700355203H1 | g2827143 | 39 | −3 | gb105eukp | Ath-B; cellulose synthase catalytic subunit | |
| 700355629H1 | g20323 | 30 | −22 | gb105pln | O. sativa RAc1 gene for actin. | |
| 700355203H1 | g2827143 | 39 | −3 | gb105eukp | Ath-B; cellulose synthase catalytic subunit | |
| 700356759H1 | g499011 | 55 | −13 | gb105pln | S. vulgare SoAc1 mRNA. | |
| 700356773H1 | g619928 | 37 | −1 | gb105eukp | hexokinase; EC 2.7.1.1 | |
| 700354406H1 | g577777 | 43 | 7 | gb105allp | glutathione peroxidase | |
| 700354672H1 | g250127 | 31 | −8 | gb105allp | 67 kda laminin receptor [human, lung cancer cell line SBC3, Peptide, 295 aa] | |
| 700356416H1 | g2330786 | 15 | 2 | gb105eukp | SPAC24C9.03; diphosphomevalonate decarboxylase | |
| 700356658H1 | g1418124 | 48 | −11 | gb105pln | Rapeseed Root mRNA for CTP:phosphocholine cytidylyltransferase, complete cds. | |
| 700355692H1 | g902583 | 31 | −38 | Zea mays clone MubG1 ubiquitin gene, complete cds. | | |
| 700356645H1 | g1911581 | 34 | 2 | gb105pln | Cyn d 1 = major allergen {clone 14c1 and CD1} [Cynodon dactylon = Bermuda grass, | |
| | | | | | pollen, mRNA Partial, 759 nt]. | |
| 700353138H1 | g22427 | 47 | −62 | gb105pln | Z. mays gene for polygalacturonase (clone 65). | |
| 700354638H1 | g1469218 | 21 | 1 | gb105pln | B. oleracea mRNA (unknown). | |
| 700354249H1 | g170772 | 80 | −10 | gb105pln | Triticum aestivum S-adenosy-L-homocysteine hydrolase (SH6.2) mRNA, complete cds. | |
| 700355293H1 | g499011 | 52 | −39 | gb105pln | S. vulgare SoAc1 mRNA. | |
| 700355789H1 | g402753 | 35 | 6 | gb105eukp | fusA; translation elongation factor EF-G | |
| 700356658H1 | g1657381 | 48 | −11 | gb105pln | P. sativum mRNA for cholinephosphate cytidylyltransferase. | |
| 700353221H1 | g168313 | 15 | −1 | gb105pln | L. perenne pollen allergen (Lol pI) gene, 3′ end. | |
| 700382711H1 | g473359 | 22 | −3 | gb105pln | P. pratense mRNA for Phl pI allergen. | |
| 700382637H1 | g416256 | 21 | −27 | gb105pln | Rice mRNA for H+-ATPase, partial sequence. | |
| 700356517H1 | g21870 | 16 | 6 | gb105pln | Wheat signal recognition particle (SRP) 7S RNA variant 1. | |
| 700353557H1 | g168490 | 38 | 9 | gb105pln | Maize glutathione S-transferase (GST-I) mRNA, complete cds. | |
| 700354309H1 | g2282583 | 98 | −94 | gb105pln | Zea mays elongatin factor 1-alpha (EF1-A) mRNA, complete cds. | |
| 700352655H1 | g248336 | 63 | −69 | gb105pln | polyubiquitin [maize, Genomic, 3841 nt]. | |
| 700355436H1 | g293888 | 65 | −74 | gb105pln | Zea mays, glyceraldehyde-3-phosphate dehydrogenase mRNA, 3′ end (clone GAPC2). | |
| 700382108H1 | g510275 | 50 | −20 | gb105eukp | DET1 | |
| 700355485H1 | g2182287 | 12 | 3 | gb105pln | Genomic sequence for Arabidopsis thaliana BAC T7N9, complete sequence. | |
| 700354640H1 | g1381150 | 13 | 4 | gb105allp | Hs-CUL-4B | |
| 700356225H1 | g22473 | 57 | −72 | gb105pln | Maize mRNA for ribulose-1,5-bisphosphate carboxylase small subunit (RuBPC; | |
| | | | | | EC 4.1.1.39). | |
| 700382609H1 | g287834 | 60 | −77 | gb105pln | Z. mays yptm2 cDNA. | |
| 700383004H1 | g2634680 | 9 | 4 | gb105allp | histidinol-phosphate aminotransferase | |
| 700353209H1 | g473359 | 23 | −1 | gb105pln | P. pratense mRNA for Phl allergen. | |
| 700355071H1 | g1173556 | 17 | 16 | gb105pln | Oryza sativa Ory s 1 mRNA, complete cds. | |
| 700353258H1 | g473359 | 22 | −5 | gb105pln | P. pratense mRNA for Phl pI allergen. | |
| 700354941H1 | g2623679 | 63 | −48 | gb105pln | Zea mays calmodulin (Zmrcalm) mRNA, complete cds. | |
| 700355562H1 | g313759 | 78 | −67 | gb105pln | Z. mays hsp 70-1 gene for heat shock protein 70. | |
| 700382307H1 | g2702272 | 18 | −5 | gb105eukp | T21L14.13 | |
| 700355915H1 | g22416 | 98 | −86 | gb105pln | Z. mays mRNA for polygalacturonase (clone PG1). | |
| 700382542H1 | g2708531 | 21 | −14 | gb105pln | Nicotiana tabacum putative RNA binding protein (QRRBP-1) mRNA, partial cds. | |
| 700382805H1 | g2262159 | 18 | −9 | gb105eukp | F5J6.4; predicted protein similar to S. pombe protein C5H10.03 | |
| 700354943H1 | g2642164 | 10 | 1 | gb105allp | hypothetical protein | |
| 700383125H1 | g1071681 | 24 | −5 | gb105allp | H. sapiens mRNA for rat translocon-associated protein delta homolog | |
| 700357160H1 | g967967 | 15 | 10 | gb105pln | Brassica campestris ssp. pekinensis (clone bif11) photosystem II 10 kDa polypeptide | |
| | | | | | mRNA, complete cds. | |
| 700352712H1 | g829147 | 60 | −56 | gb105pln | Z. mays gene for cyclophilin. | |
| 700355918H1 | g1628484 | 12 | 2 | gb105allp | mitotic centromere-associated kinesin | |
| 700382072H1 | g1432082 | 23 | −3 | gb105pln | Arabidopsis thaliana Skp1p homolog mRNA, complete cds. | |
| 700354905H1 | g2662342 | 55 | −63 | gb105pln | Oryza sativa mRNA for EF-1 alpha, complete cds. | |
| 700382383H1 | g166581 | 30 | −20 | gb105pln | A. thaliana AAc1 gene encoding actin-1, complete cds. | |
| 700355582H1 | g1184991 | 16 | −10 | gb105eukp | NTGB3 | |
| 700382035H1 | g747914 | 37 | −69 | gb105pln | Z. mays CaM1 mRNA for calmodulin. | |
| 700382609H1 | g1616613 | 34 | −20 | gb105pln | N. plumbaginifolia mRNA for small GTP-binding protein, clone Np43GTP. | |
| 700356544H1 | g2264304 | 26 | 13 | gb105pln | Arabidopsis thaliana genomic DNA, chromosome 5, P1 clone: MBG8, complete sequence. | |
| 700382596H1 | g431268 | 20 | −4 | gb105eukp | UBC6; ubiquitin conjugating enzyme | |
| 700352839H1 | g531752 | 30 | −2 | gb105eukp | YTA11 | |
| 700354295H1 | g899403 | 17 | −8 | gb105eukp | CPR1; Cpr1p | |
| 700355764H1 | g432488 | 43 | −45 | gb105pln | Wheat initiation factor 1A (eIF-1A) mRNA. | |
| 700353657H1 | g288366 | 37 | −49 | gb105pln | Z. mays PGg14 gene for polygalacturonase. | |
| 700357419H1 | g553073 | 30 | −16 | gb105eukp | reverse transcriptase | |
| 700382889H1 | g176780 | 84 | −37 | gb105allp | gamma-1 globin | |
| 700382873H1 | g1296806 | 17 | −19 | gb105pln | H. vulgare xylose isomerase gene. | |
| 700353132H1 | g587499 | 43 | −28 | gb105pln | O. sativa mRNA for calcium dependent protein kinase 11. | |
| 700355976H1 | g1877218 | 30 | −17 | gb105pln | H. vulgare mRNA for serine carboxypeptidase III, CP-MIII. | |
| 700356820H1 | g1498383 | 45 | −44 | gb105pln | Zea mays actin (Maz89) gene, partial cds. | |
| 700355430H1 | g22458 | 67 | −83 | gb105pln | Z. mays pollen specific mRNA C-terminal (clone 4H7). | |
| 700382119H1 | g669002 | 20 | −2 | gb105pln | Glycine max calnexin mRNA, complete cds. | |
| 700282447H2 | g439585 | 39 | −11 | gb105pln | Hordeum vulgare calreticulin (CRH1) mRNA, partial cds. | |
| 700353849H1 | g2584827 | 12 | 10 | gb105pln | Sequence of BAC F12F1 from Arabidopsis thaliana chromosome 1, complete sequence. | |
| 700353028H1 | g2055230 | 10 | 7 | gb105eukp | src2; SRC2; soybean cold-regulated gene | |
| 700354514H1 | g22416 | 54 | −29 | gb105pln | Z. mays mRNA for polygalacturonase (clone PG1). | |
| 700354638H1 | g2584827 | 17 | −3 | gb105pln | Sequence of BAC F12F1 from Arabidopsis thaliana chromosome 1, complete sequence. | |
| 700353049H1 | g1313946 | 25 | 6 | gb105eukp | T21C9.2 | |
| 700357160H1 | g1835730 | 26 | −10 | gb105pln | Oryza sativa photosystem II 10 kDa polypeptide mRNA, complete cds. | |
| 700353557H1 | g22314 | 38 | 6 | gb105pln | Maize mRNA for GSH gluthathione S-transferase I (GST; EC 2.5.1.18). | |
| 700354655H1 | g2529228 | 34 | −25 | gb105pln | Glycine max mRNA for 6-phosphogluconate dehydrogenase, complete cds. | |
| 700356463H1 | g1049108 | 59 | 3 | gb105allp | carbonyl reducatase | |
| 700354960H1 | g902524 | 55 | 2 | gb105pln | Zea mays clone MubG10 ubiquitin fusion protein gene, complete cds. | |
| 700356409H1 | g790969 | 65 | −40 | gb105pln | Rice mRNA for aldolase C-1, complete cds. | |
| 700382424H1 | g603403 | 14 | 8 | gb105allp | Yer163cp | |
| 700282527H2 | g473359 | 19 | 8 | gb105pln | P. pratense mRNA for Phl pI allergen. | |
| 700353154H1 | g22455 | 30 | −48 | gb105pln | Z. mays pollen specific mRNA C-terminal (clone 3A6). | |
| 700355288H1 | g1616660 | 49 | −30 | gb105pln | Zea mays adenylosuccinate synthetase mRNA, complete cds. | |
| 700357147H1 | g550543 | 55 | −46 | gb105pln | A. thaliana mRNA for ribosomal protein L16. | |
| 700382263H1 | g414702 | 34 | −9 | gb105pln | H. lanatus mRNA for allergen Hol-lI. | |
| 700352904H1 | g536533 | 27 | −3 | gb105pln | Yeast (Saccharomyces cerevisiae) chromosome II reading frame ORF YBR181c. | |
| 700354984H1 | g19451 | 40 | 4 | gb105eukp | pectate lyase | |
| 700356476H1 | g22420 | 100 | −59 | gb105pln | Z. mays mRNA for polygalacturonase (clone PG2). | |
| 700353983H1 | g166923 | 32 | −10 | gb105pln | Arabidopsis thaliana ubiquitin carrier protein (UBC1) mRNA, complete cds. | |
| 700354795H1 | g1067204 | 11 | −4 | gb105eukp | SPAC1F7.02c; unknown | |
| 700382147H1 | g1938575 | 14 | −2 | gb105eukp | B0025.3 | |
| 700383107H1 | g180939 | 97 | −27 | gb105allp | cytochrome c oxidase subunit VIII precursor | |
| 700356666H1 | g168608 | 62 | −27 | gb105pln | Maize 17S ribosomal RNA gene and flanks. | |
| 700356746H1 | g473360 | 27 | 7 | gb105eukp | phl pI ; phl pI allergen | |
| 700353793H1 | g170775 | 22 | −16 | gb105pln | Wheat translation elongation factor 1 alpha-subunit (TEF1) mRNA, complete cds. | |
| 700354514H1 | g288373 | 54 | −29 | gb105pln | Z. mays PGg6 gene for polygalacturonase. | |
| 700355203H1 | g2827139 | 34 | 1 | gb105eukp | RSW1; cellulose synthase catalytic subunit | |
| 700353276H1 | g22575 | 35 | −21 | gb105pln | Potato mRNA for pyruvate kinase (cytosolic isozyme). | |
| 700354961H1 | g414908 | 28 | 5 | gb105allp | pollen allergen Phl pI | |
| 700355236H1 | g473205 | 60 | −2 | gb105pln | E. gunnii mRNA for mitochondrial malate dehydrogenase. | |
| 700353243H1 | g166873 | 26 | −2 | gb105pln | A. thaliana S-adenosylmethionine synthetase 2 (sam-2) gene, complete cds. | |
| 700353032H1 | g662367 | 59 | −27 | gb105pln | Zea mays farnesyl pyrophosphate synthetase (fps) mRNA, complete cds. | |
| 700355266H1 | g22458 | 59 | −36 | gb105pln | Z. mays pollen specific mRNA C-terminal (clone 4H7). | |
| 700356881H1 | g1518539 | 15 | −12 | gb105pln | Glycne max UDP-glucose dehydrogenase mRNA, complete cds. | |
| 700383172H1 | g1302648 | 59 | −7 | gb105allp | 37 kD laminin receptor precursor/p40 ribosome associated protein | |
| 700353987H1 | g22421 | 53 | 6 | gb105pln | Z. mays mRNA for polygalacturonase (clone PG3). | |
| 700382050H1 | g1173556 | 24 | −20 | gb105pln | Oryza sativa Ory s 1 mRNA, complete cds. | |
| 700382812H1 | g168088 | 18 | −8 | gb105eukp | rps16; ribosomal protein S16 | |
| 700353042H1 | g499015 | 39 | −22 | gb105pln | V. cardunculus mRNA for cynarase. | |
| 700353860H1 | g2160300 | 8 | 3 | gb105eukp | ribosomal protein | |
| 700383165H1 | g2072951 | 15 | −0 | gb105allp | putative p150 | |
| 700352808H1 | g1272684 | 48 | −64 | gb105pln | Z. mays mRNA for acetyl CoA carboxylase (partial). | |
| 700355433H1 | g182033 | 19 | −9 | gb105allp | elastase III A | |
| 700355424H1 | g2645162 | 28 | −3 | gb105pln | Oryza sativa mRNA, similar to initiation factor eIF-4c. | |
| 700356270H1 | g2463566 | 52 | −44 | gb105pln | Zea mays mRNA for squalene synthase, complete cds. | |
| 700382662H1 | g20309 | 18 | 5 | gb105pln | O. sativa DNA for pollen-specific gene. | |
| 700353203H1 | g2181184 | 10 | −5 | gb105eukp | JR3 protein | |
| 700356028H1 | g2345153 | 72 | −35 | gb105pln | Zea mays ribsomal protein S4 (rps4) mRNA, complete cds. | |
| 700383135H1 | g2505874 | 22 | −12 | gb105eukp | putative kinase | |
| 700354903H1 | g473360 | 33 | −5 | gb105eukp | phl pI ; Phl pI allergen | |
| 700355974H1 | g2267582 | 22 | −6 | gb105pln | Gossypium hirsutum vacuolar H+-ATPase subunit E mRNA, complete cds. | |
| 700382511H1 | g397958 | 56 | −63 | gb105pln | Z. mays mRNA for beta-amylase. | |
| 700382849H1 | g20321 | 50 | −54 | gb105pln | Oryza sativa RAc1 mRNA for actin> | |
| 700355955H1 | g2290414 | 18 | 5 | gb105allp | NADH dehydrogenase subunit 3 | |
| 700383123H1 | g313141 | 62 | −82 | gb105pln | Z. mays ZmPRO3 mRNA for profilin 3. | |
| 700355525H1 | g1009233 | 23 | −4 | gb105pln | Nicotiana tabacum SUP2 mRNA, 3′ end of cds. | |
| 700353991H1 | g1771781 | 26 | −24 | gb105pln | T. aestivum mRNA for beta-amylase. | |
| 700353511H1 | g1817578 | 63 | −36 | gb105allp | alpha-globin 2 | |
| 700352911H1 | g172559 | 10 | 2 | gb105eukp | Sec13; Sec13p | |
| 700354704H1 | g1107486 | 27 | −7 | gb105pln | A. thaliana mRNA for 60S ribosomal protein L27a. | |
| 700356492H1 | g1519250 | 60 | −32 | gb105pln | Oryza sativa GF14-c protein mRNA, complete cds. | |
| 700356939H1 | g303856 | 36 | −15 | gb105pln | Rice mRNA for ubiquitin protein fused to a ribosomal protein, complete cds. | |
| 700382173H1 | g22416 | 95 | −77 | gb105pln | Z. mays mRNA for polygalacturonase (clone PG1). | |
| 700355208H1 | g434906 | 87 | −6 | gb105eukp | S18 ribosomal protein | |
| 700382110H1 | g168315 | 9 | 12 | gb105pln | L. perenne pollen allergen (Lol pI gene, complete cds. | |
| 700352793H1 | g1200238 | 66 | −50 | gb105pln | H. vulgare mRNA for GAMby protein. | |
| 700354473H1 | g456092 | 6 | 7 | gb105eukp | degradation of polyuronides in fruit cell wall; poly [1,4-a-D-galacturonide] glycan | |
| | | | | | hydrolase; EC 3.2.1.15; polygalacturonase | |
| 700382889H1 | g183885 | 90 | −41 | gb105allp | gamma-globin | |
| 700382614H1 | g168292 | 12 | 7 | gb105allp | ORF1 | |
| 700354625H1 | g473360 | 9 | 7 | gb105allp | Phl pI allergen | |
| 700356637H1 | g313139 | 46 | −17 | gb105pln | Z. mays ZmPRO2 mRNA for profilin 2. | |
| 700282553H2 | g2463586 | 10 | 8 | gb105eukp | precursor of cysteine proteinase; FB22 precursor | |
| 700354619H1 | g558574 | 26 | −7 | gb105eukp | Eh/PPi-PFK; pyrophosphate-fructose-6-phosphate 1-phosphotransferase; EC 2.7.1.90 | |
| 700382272H1 | g22485 | 61 | −75 | gb105pln | Maize mRNA for sucrose synthase (EC 2.4.1.13). | |
| 700357426H1 | g2529462 | 7 | 5 | gb105allp | YokM | |
| 700352613H1 | g182865 | 17 | 8 | gb105allp | 4F2 cell-surface antigen | |
| 700282461H2 | g833835 | 16 | 3 | gb105allp | amygdalin hydrolase isoform AH I precursor | |
| 700356213H1 | g313137 | 49 | −46 | gb105pln | Z. mays ZmPRO1 mRNA for profilin 1. | |
| 700354992H1 | g1167835 | 20 | −15 | gb105pln | H. lanatus mRNA for Hol lI protein. | |
| 700356838H1 | g2804435 | 10 | −2 | gb105eukp | C42C1.9 | |
| 700355433H1 | g220014 | 19 | −9 | gb105allp | pancreatic protease E precursor | |
| 700355756H1 | g1403043 | 19 | −24 | gb105pln | H. chilense x T. turigidum conv. durum (Tritordeum) mRNA for S-adenosylmethionine | |
| | | | | | decarboxylase. | |
| 700382630H1 | g2239261 | 21 | −24 | gb105pln | Zea mays mRNA for pectin methylesterase-like protein. | |
| 700354459H1 | g2511587 | 36 | −36 | gb105pln | Arabidopsis thaliana mRNA for proteasome subunit prc1. | |
| 700357483H1 | g22418 | 95 | −29 | gb105pln | Z. mays gene for polygalacturonase (clone 17). | |
| 700282422H2 | g473359 | 18 | 10 | gb105pln | P. pratense mRNA for Phl pI allergen. | |
| 700382866H1 | g3265 | 25 | −9 | gb105eukp | GAL 10; UDP-galactose-4-epimerase; EC 5.1.3.2 | |
| 700353951H1 | g1764162 | 9 | 8 | gb105allp | canalicular multispecific organic anion transporter | |
| 700356032H1 | g2668743 | 51 | −51 | gb105pln | Zea mays ubiquitin conjugating enzyme (UBC) mRNA, complete cds. | |
| 700356493H1 | g1066162 | 36 | −15 | gb105pln | B. napus mRNA for glyoscysomal beta-ketoacyl-thiolase precursor. | |
| 700383128H1 | g288611 | 32 | −24 | gb105pln | Z. mays PG gene for polygalacturonase. | |
| 700382338H1 | g414702 | 38 | −21 | gb105pln | H. lanatus mRNA for allergen Hol-lI. | |
| 700382379H1 | g287829 | 94 | −76 | gb105pln | Z. mays gene for polygalacturonase. | |
| 700282523H2 | g473360 | 16 | 7 | gb105eukp | phl pI ; Phl pI allergen | |
| 700353608H1 | g1381140 | 27 | −12 | gb105eukp | cul-4; CUL-4 | |
| 700355764H1 | g2645162 | 28 | −3 | gb105pln | Oryza sativa mRNA, similar to initiation factor eIF-4c. | |
| 700383195H1 | g556557 | 37 | −62 | gb105pln | Rice mRNA for homologue of Tat binding protein, complete cds. | |
| 700352624H1 | g1817548 | 13 | −2 | gb105allp | phosphoethanolamine cytidylyltransferase | |
| 700382004H1 | g429015 | 26 | −51 | gb105pln | Rice mRNA for ribosomal protein (gene name SS498), partial cds. | |
| 700382538H1 | g2408094 | 11 | 6 | gb105eukp | SPAC8C9.11; hypothetical protein | |
| 700355291H1 | g2815123 | 17 | 2 | gb105eukp | Y45F10A.f | |
| 700382528H1 | g886400 | 26 | −12 | gb105pln | Oryza sativa MADS-box protein (MADS2) mRNA, complete cds. | |
| 700352988H1 | g288373 | 98 | −55 | gb105pln | Z. mays PGg6 gene for polygalacturonase. | |
| 700382907H1 | g499655 | 17 | 3 | gb105eukp | gamma-thionin homolog | |
| 700354692H1 | g167109 | 53 | −35 | gb105pln | Hordeum vulgare vacuolar ATPase B subunit mRNA, complete cds. | |
| 700353542H1 | g2281085 | 7 | 4 | gb105eukp | F18O19.4; CTR1 protein kinase isolog | |
| 700354471H1 | g2353344 | 12 | −11 | gb105eukp | cytochrome oxidase I | |
| 700382439H1 | g1405747 | 12 | 5 | gb105allp | PR264/SC35 | |
| 700382281H1 | g1209258 | 11 | 3 | gb105allp | protease inhibitor II | |
| 700352666H1 | g575354 | 26 | −11 | gb105pln | O. sativa SC34 mRNA for tumor suppressor. | |
| 700382016H1 | g22456 | 40 | −85 | gb105pln | Z. mays pollen specific mRNA C-terminal (clone 3C12). | |
| 700353144H1 | g2190559 | 10 | 5 | gb105eukp | F5I14.16; F5I14.16 | |
| 700353969H1 | g313139 | 45 | −36 | gb105pln | Z. mays ZmPRO2 mRNA for profilin 2. | |
| 700382002H1 | g829147 | 49 | −70 | gb105pln | Z. mays gene for cyclophilin. | |
| 700382027H1 | g16513 | 41 | −34 | gb105pln | A. thaliana gene for suppressor-like protein. | |
| 700382947H1 | g22416 | 96 | −43 | gb105pln | Z. mays mRNA for polygalacturonase (clone PG1). | |
| 700356645H1 | g293901 | 86 | −31 | gb105pln | Zea mays Zea mI gene, complete cds. | |
| 700382311H1 | g1786719 | 11 | 3 | gb105allp | o292; This 292 aa ORF is 44 pct identical (1 gap) to 287 residues of an approx. 304 aa | |
| | | | | | protein YHAE_ECOLI SW: P23523 | |
| 700353251H1 | g414702 | 21 | −5 | gb105pln | H. lanatus mRNA for allergen Hol-lI. | |
| 700356670H1 | g1304270 | 44 | −4 | gb105eukp | zha1; plasma membrane H+-ATPase; EC 3.6.1.35 | |
| 700353820H1 | g499888 | 12 | 7 | gb105eukp | ORF | |
| 700353222H1 | g1302158 | 28 | −12 | gb105eukp | RPS3 | |
| 700354517H1 | g1403188 | 22 | 7 | gb105eukp | F32D8.6 | |
| 700356229H1 | g939778 | 24 | −1 | gb105pln | Zea mays MADS-box protein (ZAG3) mRNA, complete cds. | |
| 700382812H1 | g396531 | 29 | −12 | gb105eukp | ribosomal protein S19 | |
| 700354385H1 | g1929055 | 31 | −6 | gb105pln | L. esculentum mRNA for 3,4-dihydroxy-2-butanone kinase. | |
| 700356960H1 | g836720 | 16 | 6 | gb105allp | YFL034W | |
| 700282504H2 | g1652154 | 20 | 4 | gb105allp | hypothetical protein | |
| 700355901H1 | g1495248 | 22 | −3 | gb105pln | A. thaliana mRNA for unknown protein, eRF1-3 gene. | |
| 700355945H1 | g829147 | 71 | −73 | gb105pln | Z. mays gene for cyclophilin. | |
| 700357493H1 | g19902 | 23 | 3 | gb105allp | pollen specific protein | |
| 700383039H1 | g169803 | 21 | 1 | gb105eukp | lambda-nOCI; oryzacystatin-II | |
| 700382790H1 | g22427 | 67 | −60 | gb105pln | Z. mays gene for polygalacturonase (clone 65). | |
| 700356409H1 | g168419 | 98 | −63 | gb105pln | Maize (Z. mays) aldolase mRNA, complete cds. | |
| 700354363H1 | g1498391 | 45 | −58 | gb105pln | Zea mays actin (Maz65) gene, partial cds. | |
| 700382763H1 | g2696054 | 96 | −46 | gb105allp | adducin-like protein | |
| 700355169H1 | g2828287 | 12 | −3 | gb105eukp | T18B16.90; putative protein | |
| 700282457H2 | g1718238 | 8 | 4 | gb105eukp | (1,4)-beta-xylan endohydrolase, isoenzyme X-II; EC 3.2.1.8 | |
| 700353922H1 | g218000 | 48 | −36 | gb105pln | Potato mRNA for UDP-glucose pyrophosphorylase (EC 2.7.7.9). | |
| 700354984H1 | g308902 | 40 | 4 | gb105eukp | pectate lyase | |
| 700353874H1 | g313139 | 44 | −22 | gb105pln | Z. mays ZmPRO2 mRNA for profilin 2. | |
| 700355723H1 | g160811 | 25 | −17 | gb105eukp | ribosomal protein L21; L21 | |
| 700357493H1 | g17782 | 18 | 5 | gb105allp | Bplo gene product | |
| 700382290H1 | g595955 | 27 | −0 | gb105eukp | FatB; Uc FatB2 | |
| 700353081H1 | g2795805 | 18 | 7 | gb105eukp | F17A14.3; putative protein kinase | |
| 700353810H1 | g1944572 | 29 | −14 | gb105pln | H. vulgare protein PAL mRNA for phenylalanine ammonia-lyase (1831 bp). | |
| 700382148H1 | g295850 | 58 | 6 | gb105eukp | fructose bisphosphate aldolase | |
| 700353823H1 | g2275006 | 23 | −17 | gb105pln | Hordeum vulgare mRNA for expressed sequence tag. | |
| 700353184H1 | g2618600 | 16 | −4 | gb105pln | Arabidopsis thaliana genomic DNA, chromosome 5, P1 clone: MCD12, complete | |
| | | | | | sequence. | |
| 700355951H1 | g1167835 | 20 | −2 | gb105pln | H. lanatus mRNA for Hol lI protein. | |
| 700354594H1 | g2662048 | 17 | 8 | gb105allp | receptor kinase 1 | |
| 700357164H1 | g16427 | 19 | −2 | gb105eukp | protease inhibitor II | |
| 700382491H1 | g313137 | 17 | −21 | gb105pln | Z. mays ZmPRO1 mRNA for profilin 1. | |
| 700357165H1 | g248336 | 72 | −82 | gb105pln | polyubiquitin [maize, Genomic 3841 nt]. | |
| 700352751H1 | g1321917 | 32 | −16 | gb105eukp | su12; cytoplasmic ribosomal protein S7 | |
| 700353567H1 | g1532168 | 22 | 3 | gb105eukp | AT.I.24-6 | |
| 700355576H1 | g439585 | 43 | −26 | gb105pln | Hordeum vulgare calreticulin (CRH1) mRNA, partial cds. | |
| 700353183H1 | g22427 | 24 | −42 | gb105pln | Z. mays gene for polygalacturonase (clone 65). | |
| 700354902H1 | g168398 | 49 | −39 | gb105pln | Zea mays auxin-binding protein (abp4) gene, exons 1-5 and complete cds. | |
| 700282544H2 | g507770 | 37 | −13 | gb105pln | Zea mays D3L H(+)-transporting ATPase (Mha1) gene, complete cds. | |
| 700382579H1 | g498745 | 9 | 2 | gb105allp | Pir7b | |
| 700354253H1 | g1167835 | 29 | −13 | gb105pln | H. lanatus mRNA for Hol lI protein. | |
| 700282510H2 | g168547 | 37 | −24 | gb105pln | Zea mays putative brain specific 14-3-3 protein, tau protein homolog mRNA, partial cds. | |
| 700382235H1 | g2415701 | 41 | −32 | gb105pln | P. pratense mRNA for profilin 4. | |
| 700356134H1 | g6902 | 13 | 6 | gb105eukp | unc-33; hypothetical polypeptide III | |
| 700353008H1 | g2224926 | 27 | −4 | gb105pln | Arabidopsis thaliana ethylene-insensitive3-like1 (EIL1) mRNA, complete cds. | |
| 700354402H1 | g2668745 | 59 | −23 | gb105pln | Zea mays inorganic pyrophosphatase (IPP) mRNA, complete cds. | |
| 700355437H1 | g2190553 | 31 | −14 | gb105eukp | F5I14.9 | |
| 700383194H1 | g1246116 | 14 | −5 | gb105eukp | major allergen Pha a1; major allergen Pha a1 | |
| 700354392H1 | g22418 | 81 | −73 | gb105pln | Z. mays gene for polygalacturonase (clone 17). | |
| 700352765H1 | g1498596 | 55 | −63 | gb105pln | Zea mays phospholipid transfer protein mRNA, complete cds. | |
| 700355438H1 | g22206 | 12 | 13 | gb105pln | Maize Ins1 insertion element upstream of bronze gene Bz-McC. | |
| 700356101H1 | g20321 | 35 | −8 | gb105pln | Oryza sativa RAc1 mRNA for actin. | |
| 700353113H1 | g288373 | 97 | −80 | gb105pln | Z. mays PGg6 gene for polygalacturonase. | |
| 700356074H1 | g2065436 | 6 | 8 | gb105eukp tea1p | | |
| 700356273H1 | g13014 | 62 | 5 | gb105allp | URF 5 (NADH dehydrogenase subunit) | |
| 700353810H1 | g20280 | 34 | −12 | gb105pln | Rice gene for phenylalanine ammonia-lyase (EC 4.3.1.5). | |
| 700353525H1 | g288373 | 51 | −59 | gb105pln | Z. mays PGg6 gene for polygalacturonase. | |
| 700355331H1 | g1236961 | 18 | −2 | gb105eukp | hydrolysis of the cyanogenic glucoside (R)-prunasin hydrolase precursor; EC 3.2.1.21 | |
| 700354726H1 | g416148 | 41 | −20 | gb105pln | Zea mays beta-7 tubulin (tub7) mRNA, complete cds. | |
| 700354795H1 | g530347 | 11 | −5 | gb105eukp | RNA helicase | |
| 700353643H1 | g206741 | 8 | 6 | gb105allp | ribosomal protein S12 | |
| 700353887H1 | g1246115 | 28 | −9 | gb105pln | major allergen Pha a1 [Phalaris aquatica = canary grass, pollen, mRNA Partial, | |
| | | | | | 1088 nt]. | |
| 700352769H1 | g882247 | 37 | −13 | gb105pln | Urochloa panicoides carbonic anhydrase 1 mRNA, complete cds. | |
| 700382050H1 | g293901 | 60 | −71 | gb105pln | Zea mays Zea mI gene, complete cds. | |
| 7003454511H1 | g1814400 | 35 | −0 | gb105pln | Mesembranthemum crystallinum phosphoglucomutase (PGM) mRNA, complete cds. | |
| 700353086H1 | g1458245 | 8 | 6 | gb105eukp | F54D11.1 | |
| 700354787H1 | g1177668 | 12 | 5 | gb105eukp | SPAC3H8.10; unknown | |
| 700355734H1 | g248336 | 94 | −43 | gb105pln | polyubiquitin [maize, Genomic, 3841 nt]. | |
| 700357406H1 | g531171 | 67 | 3 | gb105allp | Csa-19 | |
| 700382511H1 | g169778 | 37 | −36 | gb105pln | Oryza sativa beta-amylase gene, complete cds. | |
| 700356261H1 | g2623310 | 14 | 8 | gb105eukp | T20B5.16 | |
| 700356281H1 | g288373 | 84 | −54 | gb105pln | Z. mays PGg6 gene for polygalacturonase. | |
| 700355456H1 | g1498325 | 24 | −45 | gb105pln | Glycine max actin (Soy86) gene, partial cds. | |
| 700356821H1 | g1498389 | 47 | −63 | gb105pln | Zea mays actin (Maz81) gene, partial cds. | |
| 700353885H1 | g1438877 | 19 | −3 | gb105allp | zinc finger protein | |
| 700354652H1 | g414907 | 33 | −27 | gb105pln | P. pratense mRNA for pollen allergen PhlpI. | |
| 700355738H1 | g499655 | 9 | 5 | gb105eukp | gamma-thionin homolog | |
| 700355960H1 | g22416 | 48 | −57 | gb105pln | Z. mays mRNA for polygalacturonase (clone PG1). | |
| 700356209H1 | g293886 | 34 | 1 | gb105pln | Zea mays glyceraldehyde-3-phosphate dehydrogenase mRNA, 3′ end, (clone GAPC3). | |
| 700382436H1 | g1418578 | 6 | 5 | gb105allp | R11D1.8 | |
| 700355976H1 | g409581 | 59 | −48 | gb105pln | Rice mRNA for serine carboxypeptidase-like protein. | |
| 700382826H1 | g168608 | 55 | −62 | gb105pln | Maize 17S ribosomal RNA gene and flanks. | |
| 700382764H1 | g22183 | 86 | −81 | gb105pln | Maize mRNA for beta 2 tubulin. | |
| 700352658H1 | g288366 | 100 | −59 | gb105pln | Z. mays PGg14 gene for polygalacturonase. | |
| 700354492H1 | g1256495 | 23 | −4 | gb105eukp | r10H10.1 | |
| 700357469H1 | g288373 | 48 | −3 | gb105pln | Z. mays PGg6 gene for polygalacturonase. | |
| 700382935H1 | g1550721 | 10 | 7 | gb105eukp | HPR1; RNA binding protein | |
| 700383075H1 | g1814402 | 36 | −21 | gb105pln | Mesembryanthemum crystallinum methionine synthase (MetE) mRNA, complete cds. | |
| 700382663H1 | g2168137 | 24 | −14 | gb105eukp | PKF1 | |
| 700353582H1 | g22455 | 72 | −30 | gb105pln | Z. mays pollen specific mRNA C-terminal (clone 3A6.). | |
| 700355649H1 | g2149051 | 10 | 4 | gb105eukp | Ran3; small Ras-like GTP-binding protein | |
| 700383103H1 | g217973 | 28 | −42 | gb105pln | Zea mays gene for triosephosphate isomerase, complete cds. | |
| 700382385H1 | g2341060 | 79 | −64 | gb105pln | Zea mays translational initiation factor eIF-4A (tif-4A3) mRNA, complete cds. | |
| 700353275H1 | g20310 | 14 | −2 | gb105eukp | pollen specific gene | |
| 700352763H1 | g2154729 | 42 | −41 | gb105pln | C. dactylon mRNA for profilin 1. | |
| 700354277H1 | g220088 | 63 | −32 | gb105allp | TAXREB67 protein | |
| 700356867H1 | g1556445 | 63 | −52 | gb105pln | Hordeum vulgare alpha tubulin (tubA) mRNA, complete cds. | |
| 700353038H1 | g1871506 | 74 | −24 | gb105pln | C. dactylon mRNA for clacium-binding pollen allergen. | |
| 700354870H1 | g171996 | 9 | 4 | gb105eukp | MNS1; alpha-mannosidase | |
| 700355346H1 | g2827001 | 20 | −6 | gb105pln | Triticum aestivum 70 kDa heat shock protein (TaHSP70d) mRNA, complete cds. | |
| 700356053H1 | g2529228 | 37 | −26 | gb105pln | Glycine max mRNA for 6-phosphogluconate dehydrogenase, complete cds. | |
| 700282543H2 | g536152 | 8 | 7 | gb105allp | ORF YBL092w | |
| 700356467H1 | g602605 | 61 | −8 | gb105pln | Zea mays tandem genes for alpha1-tubulin and alpha2-tubulin. | |
| 700356445H1 | g414702 | 19 | 7 | gb105pln | H. lanatus mRNA for allergen Hol-lI. | |
| 700382342H1 | g1771781 | 26 | −33 | gb105pln | T. aestivum mRNA for beta-amylase. | |
| 700383128H1 | g288378 | 29 | −22 | gb105pln | Z. mays gene PG for polygalacturonase. | |
| 700382263H1 | g414907 | 34 | −9 | gb105pln | P. pratense mRNA for pollen allergen PhlpI. | |
| 700355770H1 | g1469218 | 31 | −17 | gb105pln | B. oleracea mRNA (unknown). | |
| 700382127H1 | g2760165 | 19 | −1 | gb105pln | Arabidopsis thaliana genomic DNA, chromosome 5, P1 clone: MAC9, complete | |
| | | | | | sequence. | |
| 700382905H1 | g2827650 | 10 | 0 | gb105eukp | F18F4.60; potassium transporter-like protein | |
| 700382050H1 | g1911581 | 22 | −16 | gb105pln | Cyn d 1 = major allergen {clone 14c1 and CD1} [Cynodon dactylon = Bermuda grass, | |
| | | | | | pollen, mRNA Partial, 759 nt]. | |
| 700353680H1 | g1184775 | 50 | −41 | gb105pln | Zea mays glyceraldehyde-3-phosphate dehydrogenase GAPC4 (gpc4) mRNA, | |
| | | | | | complete cds. | |
| 700354713H1 | g1418971 | 27 | −7 | gb105pln | H. vulgare mRNA for transcription factor EmBP-1. | |
| 700356739H1 | g22427 | 26 | −10 | gb105pln | Z. mays gene for polygalacturonase (clone 65). | |
| 700282587H2 | g313139 | 32 | −15 | gb105pln | Z. mays ZmPRO2 mRNA for profilin 2. | |
| 700353162H1 | g22427 | 48 | −58 | gb105pln | Z. mays gene for polygalacturonase (clone 65). | |
| 700353209H1 | g1167835 | 21 | 1 | gb105pln | H. lanatus mRNA for Hol lI protein. | |
| 700355762H1 | g17795 | 29 | −9 | gb105eukp | Bplo | |
| 700357452H1 | g2564048 | 26 | −27 | gb105pln | Arabidopsis thaliana genomic DNA, chromosome 5, P1 clone: MDK15, complete | |
| | | | | | sequence. | |
| 700356039H1 | g288373 | 88 | −79 | gb105pln | Z. mays PGg6 gene for polygalacturonase. | |
| 700382131H1 | g431163 | 32 | −20 | gb105pln | Lily mRNA for ORF, partial cds. | |
| 700382634H1 | g18053 | 15 | 8 | gb105pln | C. lacryma-jobi L. mRNA for gamma-coixin (22 KDa). | |
| 700383087H1 | g699510 | 24 | −11 | gb105eukp | M(1)1B; minute(1)1B protein; minute(1)1B | |
| 700382977H1 | g22458 | 53 | −36 | gb105pln | Z. mays pollen specific mRNA C-terminal (clone 4H7). | |
| 700352779H1 | g2828287 | 33 | −12 | gb105eukp | T18B16.90; putative protein | |
| 700356939H1 | g347063 | 33 | −12 | gb105pln | Brassic rapa ubiquitin/ribosomal protein mRNA, complete cds. | |
| 700354330H1 | g393449 | 23 | −15 | gb105pln | S. cereale mRNA for beta-amylase. | |
| 700355525H1 | g1079719 | 25 | −6 | gb105pln | Ricinus communis eukaryotic release factor 3 mRNA, partial cds, 3′ end. | |
| 700356039H1 | g22427 | 84 | −80 | gb105pln | Z. mays gene for polygalacturonase (clone 65). | |
| 700353164H1 | g577818 | 17 | 7 | gb105pln | Z. mays gene for H2B histone (gH2B4). | |
| 700353857H1 | g599958 | 19 | −1 | gb105eukp | chloroplast outer envelope protein 86 | |
| 700356789H1 | g1666078 | 58 | −15 | gb105pln | H. vulgare mRNA for glutamyl-tRNA reductase (2nd isoform). | |
| 700383057H1 | g20805 | 30 | −11 | gb105pln | P. sativum lpd mRNA for dihydrolipoamide dehydrogenase. | |
| 700382379H1 | g22416 | 94 | −76 | gb105pln | Z. mays mRNA for polygalacturonase (clone PG1). | |
| 700354287H1 | g1575594 | 65 | −50 | gb105pln | Triticum aestivum fimbrin/plastin-like mRNA, partial cds. | |
| 700355732H1 | g2224897 | 28 | −5 | gb105eukp | PcMYB1; binds specifically to MRE-CHS (MYB recognition element in the parsley CHS | |
| | | | | | promoter); DNA-binding protein PcMYB1 | |
| 700382348H1 | g1800216 | 20 | −16 | gb105pln | Sorghum bicolor phytochrome B (PHYB) gene, partial cds. | |
| 700354833H1 | g780371 | 23 | −28 | gb105pln | Oryza sativa enolase mRNA, complete cds. | |
| 700355295H1 | g1181330 | 87 | −14 | gb105pln | Z. mays CNX mRNA. | |
| 700382826H1 | g2114208 | 56 | −61 | gb105pln | Wheat DNA for 18S rRNA and telomere repeat sequence. | |
| 700282473H2 | g1155255 | 28 | −5 | gb105eukp | beta-glucosidase; EC 3.2.1.21 | |
| 700382025H1 | g1848211 | 23 | −31 | gb105pln | N. tabacum mRNA for protein disulfide-isomerase precursor. | |
| 700355540H1 | g793858 | 10 | 4 | gb105eukp | legumin precursor | |
| 700353715H1 | g1276652 | 14 | 12 | gb105pln | Prophyra purpurea chloroplast genome, complete sequence. | |
| 700354501H1 | g22473 | 67 | −27 | gb105pln | Maize mRNA for ribulose-1,5-bisphosphate carboxylase small subunit (RuBPC; | |
| | | | | | EC 4.1.1.39). | |
| 700352868H1 | g1151221 | 16 | 5 | gb105eukp | SSE1; Sse1p: Member of the 70-kDa heat-shock protein family | |
| 700354696H1 | g313141 | 64 | −47 | gb105pln | Z. mays ZmPRO3 mRNA for profilin 3. | |
| 700355295H1 | g2760165 | 52 | 1 | gb105pln | arabidopsis thaliana genomic DNA, chromosome 5, P1 clone: MAC9, complete | |
| | | | | | sequence. | |
| 700353047H1 | g167107 | 64 | −21 | gb105pln | Hordeum vulgare vacuolar ATPase B subunit isoform mRNA, complete cds. | |
| 700355949H1 | g168543 | 57 | −31 | gb105pln | Zea mays putative ribosomal protein S8 mRNA, partial cds. | |
| 700356819H1 | g1652132 | 9 | 7 | gb105allp | sensory transduction histidine kinase | |
| 700354259H1 | g2738247 | 31 | −37 | gb105pln | Arabidopsis thaliana cobalamin-independent methionine synthase (ATCIMS) mRNA, | |
| | | | | | complete cds. | |
| 700354961H1 | g473360 | 29 | 2 | gb105allp | Phl pI allergen | |
| 700382880H1 | g288366 | 62 | −37 | gb105pln | Z. mays PGg14 gene for polygalacturonase. | |
| 700356048H1 | g1246115 | 21 | 1 | gb105pln | major allergen Pha a 1 [Phalaris aquatica = canary grass, pollen, mRNA Partial, | |
| | | | | | 1088 nt]. | |
| 700356235H1 | g817895 | 18 | −2 | gb105eukp | unknown | |
| 700353247H1 | g1514643 | 19 | −7 | gb105eukp | multidrug resistance protein; PDR5-like ABC transporter | |
| 700354611H1 | g886024 | 9 | 6 | gb105allp | protein kinase PkwA | |
| 700354414H1 | g433040 | 38 | −56 | gb105pln | Zea mays W-22 clone PREM-1A retroelement PREM-1, partial sequence. | |
| 700382080H1 | g60072 | 11 | 7 | gb105allp | p24/p25/p7 | |
| 700354655H1 | g603220 | 36 | −26 | gb105pln | Medicago sativa 6-phosphogluconate dehydrogenase mRNA, complete cds. | |
| 700355014H1 | g886470 | 40 | −27 | gb105pln | C. roseus MetE mRNA for methionine synthase. | |
| 700354677H1 | g2088646 | 17 | −4 | gb105eukp | t28M21.9; Su1p isolog | |
| 700355323H1 | g414907 | 23 | 9 | gb105pln | P. pratense mRNA for pollen allergen PhlpI. | |
| 700383076H1 | g168313 | 18 | 4 | gb105pln | L. perenne pollen allergen (Lol pI ) gene, 3′ end. | |
| 700355540H1 | g1296437 | 12 | 2 | gb105eukp | leg18; legumin-like protein | |
| 700357422H1 | g1931652 | 33 | 4 | gb105eukp | T19D16.18; phosphatidylinositol-4-phosphate 5-kinase isolog | |
| 700353213H1 | g287829 | 79 | −67 | gb105pln | Z. mays gene for polygalacturonase. | |
| 700354260H1 | g1009720 | 10 | 1 | gb105eukp | ENOD8; nodulin | |
| 700353203H1 | g902791 | 11 | −6 | gb105eukp | ILL2; ILL2 | |
| 700355817H1 | g2344871 | 36 | −16 | gb105eukp | pap; purple acid phosphatase precursor; EC 3.1.3.2 | |
| 700352718H1 | g387908 | 20 | 4 | gb105pln | Brassica rapa S-phase-specific (BIS289) mRNA, complete cds. | |
| 700354759H1 | g499655 | 15 | 6 | gb105allp | gamma-thionin homolog | |
| 700355803H1 | g1928968 | 22 | −9 | gb105eukp | ribosomal protein 49 | |
| 700382157H1 | g312180 | 35 | −72 | gb105pln | Z. mays GapC4 gene. | |
| 700355770H1 | g2584827 | 19 | −9 | gb105pln | Sequence of BAC F12F1 from Arabidopsis thaliana chromosome 1, complete sequence. | |
| 700356069H1 | g495730 | 53 | 16 | gb105pln | Arabidopsis thaliana small Ras-like GTP-binding protein (Ran-2) mRNA, 3′ end. | |
| 700353539H1 | g1777930 | 26 | 1 | gb105eukp | SoNDPK1; nucleoside diphosphate kinase; EC 2.7.4.6 | |
| 700355501H1 | g499011 | 32 | −23 | gb105pln | S. vulgare SoAc1 mRNA. | |
| 700355750H1 | g2668745 | 25 | −41 | gb105pln | Zea mays inorganic pyrophosphatase (IPP) mRNA, complete cds. | |
| 700355748H1 | g168482 | 61 | −65 | gb105pln | Corn starch branching enzyme II mRNA, complete cds. | |
| 700383143H1 | g746500 | 14 | 0 | gb105allp | weak similarity to lupus LA proteins | |
| 700357428H1 | g295812 | 11 | 2 | gb105eukp | LAT52 | |
| 700382373H1 | g1532072 | 12 | 11 | gb105pln | Z. mays mRNA for S-adenosylmethionine decarboxylase. | |
| 700354467H1 | g1354271 | 11 | 10 | gb105pln | Arabidopsis thaliana aspartic proteinase mRNA, partial cds. | |
| 700383031H1 | g483411 | 27 | −62 | gb105pln | Zea mays calmodulin-binding protein mRNA, 3′ end. | |
| 700382250H1 | g2267583 | 14 | 1 | gb105eukp | tonoplast proton pumping; vacuolar H+-ATPase subunit E | |
| 700353611H1 | g21838 | 41 | −33 | gb105pln | T. aestivum PRK gene for ribulose-5-phosphate kinase. | |
| 700356595H1 | g22420 | 85 | −56 | gb105pln | Z. mays mRNA for polygalacturonase (clone PG2). | |
| 700382338H1 | g414907 | 37 | −19 | gb105pln | P. pratense mRNA for pollen allergen PhlpI. | |
| 700353848H1 | g1710150 | 29 | −14 | gb105pln | Arabidopsis thaliana proline iminopeptidase mRNA, complete cds. | |
| 700382148H1 | g20204 | 58 | 6 | gb105eukp | fructose-diphosphate aldolase (AA 1-358) | |
| 700355741H1 | g556672 | 46 | −45 | gb105pln | S. cereale (Halo) chloroplast mRNA for heat-shock protein. | |
| 700382812H1 | g157469 | 22 | −8 | gb105eukp | S19; ribosomal protein S19 | |
| 700354380H1 | g2654121 | 25 | 2 | gb105pln | Arabidopsis thaliana ribosomal protein L23a (AtrpL23a) mRNA, complete cds. | |
| 700355401H1 | g710295 | 7 | 4 | gb105allp | ribosomal protein L22 | |
| 700354473H1 | g288612 | 8 | 7 | gb105allp | polygalacturonase | |
| 700355323H1 | g168315 | 23 | 9 | gb105pln | L. perenne pollen allergen (Lol pI ) gene, complete cds. | |
| 700383152H1 | g21800 | 59 | −48 | gb105pln | T. aestivum L mRNA for histone H2B. | |
| 700354709H1 | g2564048 | 16 | −19 | gb105pln | Arabidopsis thaliana genomic DNA, chromosome 5, P1 clone: MKD15, complete | |
| | | | | | sequence. | |
| 700383178H1 | g1698572 | 46 | −24 | gb105allp | proteasome activator PA28 beta subunit | |
| 700354666H1 | g1419369 | 27 | −17 | gb105pln | Z. mays ZmABP3 mRNA for actin depolymerizing factor. | |
| 700355783H1 | g486279 | 11 | 6 | gb105eukp | ORF YKL160w | |
| 700382767H1 | g2388791 | 30 | −5 | gb105eukp | cytochrome oxidase I | |
| 700382320H1 | g444022 | 52 | −44 | gb105pln | N. tabacum mRNA for pyruvate kinase. | |
| 700354609H1 | g1911581 | 36 | −36 | gb105pln | Cyn d 1 = major allergen (clone 14c1 and CD1} [Cynodon dactylon = Bermuda grass, | |
| | | | | | pollen, mRNA Partial, 759 nt]. | |
| 700355216H1 | g313139 | 70 | −65 | gb105pln | Z. mays ZmPRO2 mRNA for profilin 2. | |
| 700356907H1 | g416037 | 56 | −20 | gb105pln | O. sativa rub1 mRNA for polybutiquitin. | |
| 700355668H1 | g1747418 | 11 | −3 | gb105pln | Z. mays mRNA for eukaryotic initiation factor-5. | |
| 700352824H1 | g2384669 | 13 | 6 | gb105eukp | AtKT1; putative potassium transporter AtKT1p | |
| 700353961H1 | g22416 | 51 | −59 | gb105pln | Z. mays mRNA for polygalacturonase (clone PG1). | |
| 700355862H1 | g2154727 | 49 | −38 | gb105pln | C. dactylon mRNA for profilin 2. | |
| 700382173H1 | g288373 | 95 | −77 | gb105pln | Z. mays PGg6 gene for polygalacturonase. | |
| 700353239H1 | g1932925 | 12 | −6 | gb105eukp | putative pectin methylesterase | |
| 700356141H1 | g2262105 | 8 | 1 | gb105eukp | T19F06.8 | |
| 700355780H1 | g205532 | 18 | 7 | gb105allp | metallothionein 2 | |
| 700354406H1 | g488476 | 43 | 7 | gb105allp | glutathione peroxidase | |
| 700356273H1 | g13370 | 56 | 3 | gb105allp | reading frame protein 5 | |
| 700383087H1 | g458972 | 26 | −6 | gb105eukp | F37C12.4 | |
| 700353261H1 | g22416 | 60 | −48 | gb105pln | Z. mays mRNA for polygalacturonase (clone PG1). | |
| 700352771H1 | g170919 | 38 | −30 | gb105pln | Yeast (Candida maltosa) ribosomal protein L41 (LEL41) gene, complete cds. | |
| 700355806H1 | g405885 | 17 | 4 | gb105allp | yeiN | |
| 700352827H1 | g170899 | 18 | −1 | gb105eukp | peroxisomal membrane protein (PMP20A) | |
| 700354485H1 | g939784 | 33 | −12 | gb105pln | Zea mays MADS-box protein (ZAP1) mRNA, complete cds. | |
| 700355258H1 | g21698 | 41 | 15 | gb105pln | T. aestivum gene for cathepsin B (A116). | |
| 700382122H1 | g313135 | 61 | −40 | gb105pln | Z. mays mRNA for porin. | |
| 700383006H1 | g2511530 | 13 | 13 | gb105pln | Eleusine indica alpha tubulin 1 (TUA1) mRNA, complete cds. | |
| 700352783H1 | g1184167 | 24 | −6 | gb105eukp | knolle | |
| 700383076H1 | g1246115 | 17 | 3 | gb105pln | major allergen Pha a 1 [Phalaris aquatica = canary grass, pollen, mRNA Partial, | |
| | | | | | 1088 nt]. | |
| 700354473H1 | g479088 | 7 | 6 | gb105allp | polygalacturonase | |
| 700353883H1 | g533692 | 22 | 1 | gb105eukp | protease inhibitor | |
| 700382013H1 | g623547 | 12 | −1 | gb105allp | queuine tRNA-ribosyltransferase | |
| 700355237H1 | g2618605 | 13 | 10 | gb105pln | Arabidopsis thaliana genomic DNA, chromosome 5, P1 clone: MUK11, complete | |
| | | | | | sequence. | |
| 700356180H1 | g2275006 | 39 | −21 | gb105pln | Hordeum vulgare mRNA for expressed sequence ta. | |
| 700356213H1 | g453975 | 39 | −5 | gb105pln | P. pratense mRNA for profilin-Phl p XI. | |
| 700356893H1 | g2738247 | 25 | 0 | gb105pln | Arabidopsis thaliana cobalamin-independent methionine synthase (ATCIMS) mRNA, | |
| | | | | | complete cds. | |
| 700352951H1 | g248336 | 53 | −10 | gb105pln | polyubiquitin [maize, Genomic 3841 nt]. | |
| 700355742H1 | g600708 | 10 | 5 | gb105eukp | unknown; YSC84 | |
| 700382329H1 | g287829 | 99 | −84 | gb105pn | Z. mays gene for polygalacturonase. | |
| 700355018H1 | g22458 | 75 | −78 | gb105pln | Z. mays pollen specific mRNA C-terminal (clone 4H7). | |
| 700355341H1 | g2370460 | 31 | −63 | gb105pln | Sorghum bicolor mRNA for putative glycoprotein. | |
| 700355419H1 | g596077 | 64 | −51 | gb105pln | Zea mays thiamaine biosynthetic enzyme (thi1-1) mRNA, complete cds. | |
| 700382966H1 | g2832632 | 35 | −9 | gb105eukp | F13C5.210; hypothetical protein | |
| 700353557H1 | g168487 | 38 | 13 | gb105pln | Maize glutathione S-transferase gene (GST-I), exons 2 and 3. | |
| 700354726H1 | g493724 | 40 | −21 | gb105pln | O. sativa mRNA for beta-tubulin. | |
| 700382883H1 | g639721 | 25 | −30 | gb105pln | Zea mays calcium-dependent protein kinase (CDPK) gene, exons 1-7 and partial cds. | |
| 700382112H1 | g168482 | 43 | −78 | gb105pln | Corn starch branching enzyme II mRNA, complete cds. | |
| 700352812H1 | g596079 | 30 | −71 | gb105pln | Zea mays thiamine biosynthetic enzyme (thi1-2) mRNA, complete cds. | |
| 700352848H1 | g288611 | 50 | −36 | gb105pln | Z. mays PG gene for polygalacturonase. | |
| 700352973H1 | g414908 | 23 | 1 | gb105eukp | pollen allergen Phl pI | |
| 700354771H1 | g555616 | 11 | −5 | gb105eukp | cim1; cytokinin induced message | |
| 700356002H1 | g1498243 | 13 | 1 | gb105eukp | putative, YGL141w | |
| 700353163H1 | g1498052 | 66 | 11 | gb105pln | Zea mays ribosomal protein S8 mRNA, complete cds. | |
| 700354594H1 | g2735017 | 19 | 7 | gb105allp | KI domain interacting kinase 1 | |
| 700354672H1 | g289984 | 31 | −8 | gb105allp | 33 kDa protein | |
| 700355424H1 | g2565420 | 34 | −18 | gb105pln | Onobrychis viciifolia eukaryotic translation initiation factor eIF-1A mRNA, | |
| | | | | | complete cds. | |
| 700354511H1 | g1881692 | 65 | −21 | gb105pln | Zea mays phosphoglucomutase mRNA, partial cds. | |
| 700382993H1 | g1008185 | 16 | 2 | gb105eukp | MTR4 | |
| 700352906H1 | g1438880 | 24 | −10 | gb105pln | Glycine max choline kinase GmCK2p mRNA, complete cds. | |
| 700355006H1 | g20323 | 18 | 3 | gb105pln | O. sativa RAc1 gene for actin. | |
| 700353026H1 | g1145693 | 44 | −0 | gb105eukp | ACT1; actin | |
| 700353536H1 | g1167836 | 30 | 7 | gb15eukp | hol lI; major group I allergen; protin with incomplete signal sequence | |
| 700354824H1 | g1388076 | 23 | −3 | gb105eukp | TRX3; thioredoxin h | |
| 700354910H1 | g157564 | 29 | −7 | gb105eukp | Aats-glupro; transfer RNA-Glu-Pro aminoacyl synthetase | |
| 700382133H1 | g2245378 | 39 | −2 | gb105eukp | ARF1; auxin response factor 1 | |
| 700382246H1 | g22427 | 61 | −69 | gb105pln | Z. mays gene for polygalacturonase (clone 65). | |
| 700353148H1 | g639833 | 38 | −11 | gb105pln | S. tuberosum mRNA for NADH dehydrogenase, NADH-binding subunit (complex I). | |
| 700355338H1 | g426441 | 35 | −21 | gb105pln | Rice mRNA for thioredoxin h, complete cds. | |
| 700382053H1 | g535141 | 9 | 5 | gb105allp | dolichyl-phosphate beta-glucosyltransferase | |
| 700353995H1 | g288373 | 80 | −7 | gb105pln | Z. mays PGg6 gene for polygalacturonase. | |
| 700355974H1 | g1263910 | 24 | −10 | gb105pln | S. oleracea mRNA for vacuolar H(+)-ATPase. | |
| 700383173H1 | g1136782 | 23 | −19 | gb105pln | Fission Yeast mRNA for elongation factor 1 alpha-A, complete cds. | |
| 700355791H1 | g1653622 | 14 | 6 | gb105allp | hypothetical protein | |
| 700357168H1 | g288366 | 83 | −90 | gb105pln | Z. mays PGg14 gene for polygalacturonase. | |
| 700352977H1 | g758354 | 34 | −0 | gb105pln | Z. mays mRNA for plasma membrane H+ ATPase. | |
| 700382226H1 | g1255428 | 6 | 8 | gb105eukp | ZC513.6 | |
| 700354583H1 | g1431254 | 48 | −5 | gb105eukp | DHH1 | |
| 700355533H1 | g1658321 | 30 | −26 | gb105pln | S. tuberosum mRNA for transketolase. | |
| 700352951H1 | g1839582 | 45 | 11 | gb105pln | polyubiquitin homolog {clone CHEM 6} [Zea mays = maize, cv, INRA 258, mercuric | |
| | | | | | chloride-treated, leaves, mRNA Partial, 199 nt, segment 1 of 2]. | |
| 700354479H1 | g1235716 | 17 | −1 | gb105pln | Arabidopsis thaliana mRNA for calcium-dependent protein kinase (CDPK), | |
| | | | | | complete cds. | |
| 700356565H1 | g19902 | 10 | −2 | gb105eukp | pollen specific protein | |
| 700356585H1 | g1864002 | 37 | −26 | gb105pln | Nicotiana tabacum mRNA for 21D7, complete cds. | |
| 700355035H1 | g396209 | 30 | −11 | gb105pln | S. polyrrhiza mRNA for D-myo-inositol-3-phosphate synthase. | |
| 700355952H1 | g1246115 | 25 | −8 | gb105pln | major allergen Pha a1 [Phalaris aquatica = canary grass, pollen, mRNA Partial, | |
| | | | | | 1088 nt]. | |
| 700382767H1 | g2388787 | 30 | −5 | gb105eukp | cytochrome oxidase I | |
| 700355268H1 | g1359893 | 25 | −24 | gb105pln | H. tuberosus mRNA for NADPH-ferrihemoprotein reductase. | |
| 700352627H1 | g2673914 | 38 | −22 | gb105eukp | T24P15.14 | |
| 700354852H1 | g1835728 | 20 | −4 | gb105pln | Oryza sativa ribosomal protein mRNA, complete cds. | |
| 700356565H1 | g17782 | 12 | −3 | gb105eukp | bplo | |
| 700354366H1 | g498040 | 28 | −23 | gb105eukp | ORF | |
| 700354754H1 | g2440191 | 38 | −12 | gb105eukp | hypothetical protein | |
| 700356240H1 | g2529375 | 42 | −32 | gb105pln | Spinacia oleracea chloroplast sedoheptulose-1,7-bisphosphatase mRNA, complete cds. | |
| 700355069H1 | g499011 | 32 | −0 | gb105pln | S. vulgare SoAc1 mRNA. | |
| 700352905H1 | g1167527 | 30 | −11 | gb105eukp | Cytb; cytochrome b | |
| 700354383H1 | g2459445 | 16 | −5 | gb105eukp | F4P9.18; putative ribonucleoprotein | |
| 700356576H1 | g1403557 | 38 | 3 | gb105eukp | P2591 protein | |
| 700354710H1 | g303854 | 42 | −58 | gb105pln | Rice mRNA for ribosomal protein L7A, complete cds. | |
| 700354786H1 | g1167836 | 18 | 2 | gb105allp | protein with incomplete signal sequence | |
| 700356485H1 | g248338 | 89 | −53 | gb105pln | polyubiquitin [maize, Genomic, 3439 nt]. | |
| 700382491H1 | g453975 | 15 | −16 | gb105pln | P. pratense mRNA for profilin-Ph1 p XI. | |
| 700353180H1 | g577611 | 69 | −74 | gb105pln | Zea mays CRT1 gene for calcium-binding protein. | |
| 700356739H1 | g22456 | 26 | −9 | gb105pln | Z. mays pollen specific mRNA C-terminal (clone 3C12). | |
| 700356775H1 | g298018 | 46 | −9 | gb105pln | M. sativa mRNA msk7 for map kinase. | |
| 700355415H1 | g1835730 | 21 | 2 | gb105pln | Oryza sativa photosystem II 10 kDa polypeptide mRNA, complete cds. | |
| 700355764H1 | g2565420 | 35 | −20 | gb105pln | Onobrychis viciifolia eukaryotic translation initiation factor eIF-1A mRNA, | |
| | | | | | complete cds. | |
| 700353067H1 | g2317728 | 26 | −1 | gb105pln | Arabidospis thaliana reversibly glycosylated polypeptide-1 (AtRGP) mRNA, | |
| | | | | | complete cds. | |
| 700355456H1 | g1498387 | 31 | −62 | gb105pln | Zea mays actin (Maz83) gene, partial cds. | |
| 700356864H1 | g169776 | 14 | 14 | gb105pln | Oryza sativa beta-amylase gene, complete cds. | |
| 700356055H1 | g22427 | 49 | −63 | gb105pln | Z. mays gene for polygalacturonase (clone 65). | |
| 700383006H1 | g22153 | 48 | −48 | gb105pln | Z. mays (B37) mRNA for alpha-tubulin 4. | |
| 700382134H1 | g992962 | 25 | −8 | gb105eukp | thioredoxin | |
| 700354307H1 | g218162 | 55 | −19 | gb105pln | Rice mRNA for ferredoxin-NADP+ reductase, complete cds. | |
| 700355489H1 | g407474 | 34 | −22 | gb105pln | P. sativium, mRNA for T-protein of glycine decarboxylase complex. | |
| 700355754H1 | g414907 | 23 | −30 | gb105pln | P. pratense mRNA for pollen allergen PhlpI. | |
| 700282508H2 | g21955 | 9 | −6 | gb105eukp | beta-glucosidase; EC 3.2.1.21 | |
| 700355854H1 | g2275206 | 7 | 5 | gb105eukp | T08I13.12; pectinesterase isolog | |
| 700382779H1 | g218082 | 48 | −12 | gb105pln | Rice mRNA for initiation factor eIF-4D (225 gene), partial sequence. | |
| 700355883H1 | g22423 | 97 | −94 | gb105pln | Z. mays mRNA for polygalacturonase (clone PG4). | |
| 700282527H2 | g1167835 | 20 | 9 | gb105pln | H. lanatus mRNA for Hol lI protein. | |
| 700383038H1 | g2131104 | 34 | −17 | gb105eukp | ORF YJR094w-a | |
| 700354581H1 | g2586333 | 40 | −1 | gb105eukp | LeKAP alpha; importin alpha | |
| 700356933H1 | g1495259 | 23 | 6 | gb105eukp orf04 | | |
| 700356653H1 | g288373 | 38 | −12 | gb105pln | Z. mays PGg6 gene for polygalacturonase. | |
| 700382502H1 | g22458 | 61 | −11 | gb105pln | Z. mays pollen specific mRNA C-terminal (clone 4H7). | |
| 700356445H1 | g168315 | 21 | 7 | gb105pln | L. perenne pollen allergen (Lol pI) gene, complete cds. | |
| 700354871H1 | g2529339 | 63 | −70 | gb105pln | Zea maize AGAMOUS homologue mRNA, complete cds. | |
| 700382883H1 | g1632767 | 19 | −14 | gb105pln | Maize mRNA for calcium dependent protein kinase, complete cds. | |
| 700352637H1 | g313760 | 75 | −79 | gb105pln | Z. mays hsp 70-2 gene for heat shock protein 70. | |
| 700382309H1 | g22458 | 92 | −69 | gb105pln | Z. mays pollen specific mRNA C-terminal (clone 4H7). | |
| 700354325H1 | g1044865 | 54 | −40 | gb105pln | C. rubrum actin gene | |
| 700355323H1 | g1167835 | 23 | 10 | gb105pln | H. lanatus mRNA for Hol lI protein. | |
| 700355313H1 | g2511530 | 53 | −53 | gb105pln | Eleusine indica alpha tubulin 1 (TUA1) mRNA, complete cds. | |
| 700353960H1 | g510910 | 60 | −42 | gb105pln | L. temulentum mRNA for histone H3. | |
| 700282457H2 | g1813595 | 8 | −1 | gb105eukp | xylan endohydrolase isoenzyme X-I; EC 3.2.1.8 | |
| 700353142H1 | g2055230 | 12 | 5 | gb105eukp | src2; SRC2; soybean cold-regulated gene | |
| 700282470H2 | g2285800 | 12 | 5 | gb105eukp | 26S proteasome beta subunit | |
| 700356510H1 | g22425 | 26 | −47 | gb105pln | Z. mays gene for polygalacturonase (clone 47). | |
| 700353210H1 | g2160155 | 27 | −23 | gb105pln | Sequence of BAC F21M12 from Arabidopsis thaliana chromosome 1, complete | |
| | | | | | sequence. | |
| 700356136H1 | g862309 | 25 | −39 | gb105pln | Rice G protein alpha-subunit (RGA1) mRNA, complete cds. | |
| 700353961H1 | g287829 | 51 | −58 | gb105pln | Z. mays gene for polygalacturonase. | |
| 700382127H1 | g669002 | 30 | −10 | gb105pln | Glycine max calnexin mRNA, complete cds. | |
| 700382935H1 | g1657691 | 10 | 7 | gb105eukp | HRP1; Hrp1p | |
| 700352770H1 | g1177320 | 34 | −15 | gb105eukp | efa27; EFA27 for EF hand, abscisic acid, 27 kD | |
| 700355509H1 | g644491 | 51 | −79 | gb105pln | Corn mRNA for elongation factor 1A. | |
| 700356073H1 | g287829 | 40 | −60 | gb105pln | Z. mays gene for polygalacturonase. | |
| 700382758H1 | g2369877 | 51 | −42 | gb105pln | Hordeum vulgare mRNA for m12 protein. | |
| 700353102H1 | g288373 | 96 | −59 | gb105pln | Z. mays PGg6 gene for polygalacturonase. | |
| 700352962H1 | g414703 | 32 | −5 | gb105eukp | allergen Hol-lI | |
| 700356104H1 | g2804593 | 19 | −17 | gb105pln | Sequence of BAC T12M4 from Arabidopsis thaliana chromosome 1, complete sequence. | |
| 700356125H1 | g2624416 | 52 | −43 | gb105pln | Zea mays mRNA for ubiquitin carrier protein UBC7. | |
| 700356134H1 | g1279329 | 17 | −1 | gb105eukp | R06C7.3 | |
| 700382643H1 | g2708531 | 25 | −12 | gb105pln | Nicotiana tabacum putative RNA binding protein (ZRRBP-1) mRNA, partial cds. | |
| 700352707H1 | g2660771 | 28 | −40 | gb105pln | Spinacia oleracea cytosolic heat shock 70 protein (HSC70-1) gene, complete cds. | |
| 700354559H1 | g786115 | 11 | −1 | gb105eukp | H(+)-transporting ATPase | |
| 700355223H1 | g1486286 | 34 | −36 | gb105pln | C. sativus mRNA for T-complex polypeptide 1. | |
| 700353294H1 | g895891 | 12 | −2 | gb105eukp | RPS5; ribosomal protein S5 | |
| 700353528H1 | g22420 | 90 | −83 | gb105pln | Z. mays mRNA for polygalacturonase (clone PG2). | |
| 700354319H1 | g1155216 | 27 | −11 | gb105pln | Lycopersicon esculentum Pto kinase interactor 1 (Pti1) mRNA, complete cds. | |
| 700354939H1 | g2262105 | 24 | −6 | gb105eukp | T19F06.8 | |
| 700383044H1 | g1165205 | 14 | −11 | gb105pln | Arabidopsis thaliana multiubiquitin chain binding protein (MBP1) mRNA, complete cds. | |
| 700383102H1 | g288373 | 44 | −33 | gb105pln | Z. mays PGg6 gene for polygalacturonase. | |
| 700352848H1 | g288378 | 35 | −22 | gb105pln | Z. mays gene PG for polygalacturonase. | |
| 700356143H1 | g255569 | 14 | 6 | gb105allp | pollen specific protein [Zea mays = corn, Peptide, 170 aa] | |
| 700383032H1 | g170750 | 16 | −7 | gb105pln | Wheat (clone p28-1) initiation factor isozyme 4F p82 subunit mRNA, complete cds. | |
| 700382518H1 | g168486 | 38 | 10 | gb105pln | Maize glutathione S-transferase gene (GST-I), exon 1. | |
| 700353042H1 | g1255684 | 54 | −41 | gb105pln | Rice mRNA for aspartic protease, complete cds. | |
| 700382445H1 | g1513227 | 21 | −6 | gb105pln | Brassica napus myo-inositol 1-phosphate synthase mRNA, complete cds. | |
| 700355030H1 | g169537 | 44 | −35 | gb105pln | Potato pyrophosphate-frustose 6-phosphate 1-phosphotransferase (PFP) alpha-subunit | |
| | | | | | mRNA, complete cds. | |
| 700382873H1 | g1296808 | 29 | −27 | gb105pln | H. vulgare mRNA for xylose isomerase. | |
| 700357426H1 | g2634366 | 8 | 4 | gb105allp | similar to adenosylmethionine-8-amino-7-oxononanoate aminotransferase | |
| 700354402H1 | g2706449 | 21 | 15 | gb105pln | Solanum tuberosum mRNA for magnesium dependent soluble inorganic pyrophosphatase. | |
| 700357479H1 | g435542 | 62 | −22 | gb105pln | Z. mays mRNA for calmodulin. | |
| 700382824H1 | g777757 | 62 | −49 | gb105pln | Saccharum hybrid (clone SCUBI561) polyubiquitin mRNA, complete cds. | |
| 700282485H2 | g2351062 | 12 | 16 | gb105pln | Arabidopsis thaliana genomic DNA, chromosome 5, P1 clone: MAH20. | |
| 700356223H1 | g22614 | 27 | −46 | gb105pln | S. vulgare pepC gene for PEP carboxylase. | |
| 700382993H1 | g1236106 | 14 | −4 | gb105eukp | W08D2.7 | |
| 700354893H1 | g1204225 | 18 | −8 | gb105eukp | SPAC56F8.03; unknown | |
| 700355815H1 | g2191137 | 6 | 6 | gb105eukp | A_IG002N01.17 | |
| 700382032H1 | g1834353 | 16 | 6 | gb105eukp | 194; hypothetical protein 194 | |
| 700382804H1 | g22425 | 35 | −14 | gb105pln | Z. mays gene for polygalacturonase (clone 47). | |
| 700354910H1 | g1419467 | 30 | −8 | gb105eukp | ZC434.5 | |
| 700355842H1 | g1255959 | 7 | 6 | gb105allp | ORF N0840 | |
| 700382886H1 | g22418 | 50 | −57 | gb105pln | Z. mays gene for polygalacturonase (clone 17). | |
| 700353221H1 | g168315 | 14 | −1 | gb105pln | L. perenne pollen allergen (Lol pI ) gene, complete cds. | |
| 700354923H1 | g1532047 | 46 | −31 | gb105pln | O. sativa mRNA for S-adenosylmethionine decarboxylase. | |
| 700352982H1 | g22149 | 100 | −62 | gb105pln | Z. mays mRNA for alpha-tubulin 3. | |
| 700354788H1 | g1218042 | 27 | −15 | gb105eukp | PAP1; secreted purple acid phosphatase precursor | |
| 700356442H1 | g22416 | 98 | −60 | gb105pln | Z. mays mRNA for polygalacturonase (clone PG1). | |
| 700382768H1 | g414702 | 33 | −4 | gb105pln | H. lanatus mRNA for allergen Hol-lI. | |
| 700353068H1 | g248338 | 70 | −53 | gb105pln | polyubiquitin [maize, Genomic, 3439 nt]. | |
| 700354550H1 | g857573 | 41 | 1 | gb105pln | Oryza sativa vacuolar H+-ATPase (vatp-P1) mRNA, complete cds. | |
| 700356977H1 | g2760172 | 37 | −3 | gb105pln | Arabidopsis thaliana genomic DNA, chromosome 5, P1 clone: MUB3, complete | |
| | | | | | sequence. | |
| 700383048H1 | g1850540 | 18 | −5 | gb105eukp | ribosomal P2 phosphoprotein | |
| 700382673H1 | g1389847 | 30 | −16 | gb105eukp | myosin regulatory light chain | |
| 700356794H1 | g2842474 | 26 | 0 | gb105pln | Arabidopsis thaliana DNA chromosome 4, BAC clone F21O9 (ESSAII project). | |
| 700353077H1 | g22458 | 62 | −69 | gb105pln | Z. mays pollen specific mRNA C-terminal (clone 4H7). | |
| 700382226H1 | g1000472 | 6 | 7 | gb105eukp | C09G9.6 | |
| 700353635H1 | g1694620 | 48 | −15 | gb105pln | Cucurbita sp. mRNA for 3-keotacyl-CoA thiolase, complete cds. | |
| 700352772H1 | g22237 | 84 | −72 | gb105pln | Maize mRNA for cytosolic GAPDH (GapC) glyceraldehyde-3-phosphate dehydrogenase. | |
| 700355969H1 | g218176 | 29 | −26 | gb105pln | Rice mRNA for 21 kd polypeptide. | |
| 700356472H1 | g1753197 | 13 | 7 | gb105allp | dipeptidyl peptidase IV | |
| 700353508H1 | g624958 | 32 | −22 | gb105pln | C. reinhardtii mRNA for ribosomal protein S27. | |
| 700382327H1 | g168270 | 26 | −7 | gb105eukp | F-1-ATPase delta subunit precursor (EC 3.6.1.3) | |
| 700354919H1 | g22237 | 32 | 12 | gb105pln | Maize mRNA for cytosolic GAPDH (GapC) glyceraldehyde-3-phosphate dehydrogenase. | |
| 700355988H1 | g1628622 | 13 | −2 | gb105eukp | FLS1; catalyzes the conversion of dihydroflavonols to flavonols; flavonol synthase | |
| 700356216H1 | g2388565 | 17 | 4 | gb105allp | Similar to Prunus pectinesterase (gb\|X95991). | |
| 700382957H1 | g288373 | 28 | −28 | gb105pln | Z. mays PGg6 gene for polygalacturonase. | |
| 700383165H1 | g2072953 | 15 | 0 | gb105allp | putative p150 | |
| 700354621H1 | g22458 | 88 | −79 | gb105pln | Z. mays pollen specific mRNA C-terminal (clone 4H7). | |
| 700357138H1 | g288366 | 76 | −73 | gb105pln | Z. mays PGg14 gene for polygalacturonase. | |
| 700353042H1 | g18903 | 53 | −41 | gb105pln | Barley mRNA for aspartic proteinase. | |
| 700354523H1 | g619788 | 28 | 4 | gb105allp | L21 ribosomal protein | |
| 700353793H1 | g949877 | 22 | −17 | gb105pln | H. vulgare mRNA for elongation factor 1-alpha. | |
| 700354414H1 | g433041 | 40 | −58 | gb105pln | Zea mays W-22 clone PREM-1B retroelement PREM-1, partial sequence. | |
| 700353643H1 | g54006 | 8 | 6 | gb105allp | ribosomal protein S12 (AA 1-132) | |
| 700356223H1 | g22415 | 19 | −27 | gb105pln | Maize mRNA for phosphoenolpyruvate carboxylase (PEPCase EC 4.1.1.31). | |
| 700355560H1 | g293901 | 51 | −66 | gb105pln | Zea mays Zea mI gene, complete cds. | |
| 700357127H1 | g17645 | 59 | −6 | gb105allp | RIBOSOMAL PROTEIN L35a | |
| 700382087H1 | g1147599 | 7 | 7 | gb105allp | dodo | |
| 700382168H1 | g169818 | 36 | −71 | gb105pln | Rice 25S ribosomal RNA gene. | |
| 700356685H1 | g22420 | 93 | −38 | gb105pln | Z. mays mRNA for polygalacturonase (clone PG2). | |
| 700383151H1 | g21493 | 32 | −14 | gb105eukp | mpp; mitochondrial processing peptidase | |
| 700355046H1 | g1638836 | 54 | −49 | gb105pln | H. vulgare mRNA for alpha-tubulin 2. | |
| 700383143H1 | g2773235 | 11 | 2 | gb105allp | contains similarity to lupus LA protein homologs | |
| 700354640H1 | g1381148 | 11 | 6 | gb105allp | Hs-CUL-4A | |
| 700356993H1 | g1203832 | 24 | −6 | gb105eukp | beta-D-glucan exohydrolase, isoenzyme ExoII; EC 3.2.1.58 | |
| 700355848H1 | g2760168 | 15 | 4 | gb105pln | Arabidopsis thaliana genomic DNA, chromosome 5, P1 clone: MEE6, complete | |
| | | | | | sequence. | |
| 700356084H1 | g288366 | 49 | −58 | gb105pln | Z. mays PGg14 gene for polygalacturonase. | |
| 700354367H1 | g22606 | 16 | −26 | gb105pln | H. vulgare mRNA for 14-3-3 protein homologue (14-3-3a). | |
| 700355895H1 | g433043 | 16 | 15 | gb105pln | Zea mays W-22 clone PREM-1E retroelement PREM-1, partial sequence. | |
| 700354893H1 | g1326056 | 19 | −7 | gb105eukp | FUN12; Fun12p: 97 kDa protein, function unknown | |
| 700354295H1 | g2443755 | 28 | −13 | gb105eukp | CYP5; cyclophilin | |
| 700355767H1 | g2190549 | 13 | −4 | gb105eukp | F5I14.17 | |
| 700382574H1 | g22425 | 27 | −14 | gb105pln | Z. mays gene for polygalacturonase (clone 47). | |
| 700354650H1 | g22458 | 87 | −72 | gb105pln | Z. mays pollen specific mRNA C-terminal (clone 4H7). | |
| 700282544H2 | g758354 | 43 | −18 | gb105pln | Z. mays mRNA for plasma membrane H+ ATPase. | |
| 700354931H1 | g288366 | 98 | −61 | gb105pln | Z. mays PGg14 gene for polygalacturonase. | |
| 700353008H1 | g2224932 | 28 | −5 | gb105pln | Arabidopsis thaliana ethylene-insensitive3 (EIN3) mRNA, complete cds. | |
| 700354341H1 | g2529657 | 18 | 8 | gb105pln | Arabidopsis thaliana chromosome II BAC T30B22 genomic sequence, complete | |
| | | | | | sequence. | |
| 700282553H2 | g2463588 | 10 | 7 | gb105allp | FB1035 precursor | |
| 700382250H1 | g1143394 | 14 | −2 | gb105eukp | V-type proton-ATPase | |
| 700356013H1 | g22427 | 37 | −83 | gb105pln | Z. mays gene for polygalacturonase (clone 65). | |
| 700383140H1 | g2632156 | 20 | −2 | gb105pln | Aspergillus niger mRNA for ribosomal protein L15. | |
| 700356582H1 | g2706460 | 18 | 7 | gb105eukp | SPAC3G9.09c; hypothetical translational initiation factor 2 alpha | |
| 700282526H2 | g1049306 | 36 | −8 | gb105pln | Arabidopsis thaliana actin-2 mRNA, complete cds. | |
| 700355245H1 | g1771781 | 52 | −44 | gb105pln | T. aestivum mRNA for beta-amylase. | |
| 700357161H1 | g556672 | 13 | −25 | gb105pln | S. cereale (Halo) chloroplast mRNA for heat-shock protein. | |
| 700383107H1 | g162901 | 55 | −16 | gb105allp | cytochrome c oxidase subunit CIX precursor (E.C. 1.9.3.1) | |
| 700382425H1 | g1495251 | 53 | 7 | gb105eukp | heat-shock protein | |
| 700382165H1 | g1575127 | 94 | −82 | gb105pln | Zea mays lumenal binding protein cBiPe2 mRNA, complete cds. | |
| 700354469H1 | g736271 | 42 | −27 | gb105pln | O. sativa hsp70 gene for heat shock protein 70. | |
| 700382342H1 | g393449 | 27 | −32 | gb105pln | S. cereale mRNA for beta-amylase. | |
| 700356858H1 | g1143864 | 7 | 0 | gb105eukp | catalyzes the release of either giberellin or cyanogenic substances from their | |
| | | | | | glucoconjugates; beta glucosidase; EC 3.2.1.21 | |
| 700356517H1 | g475886 | 28 | −4 | gb105pln | Rice mRNA for signal recognition particle 7S RNA, partial sequence. | |
| 700282564H2 | g1905943 | 30 | −24 | gb105pln | Sorghum bicolor MADS box transcription factor SbMADS1 mRNA, complete cds. | |
| 700356453H1 | g397958 | 36 | 4 | gb105pln | Z. mays mRNA for beta-amylase. | |
| 700357153H1 | g1184775 | 49 | −87 | gb105pln | Zea mays glyceraldehyde-3-phosphate dehydrogenase GAPC4 (gpc4) mRNA, | |
| | | | | | complete cds. | |
| 700382985H1 | g2827711 | 15 | −2 | gb105eukp | F6H11.130; oxoglutarate dehydrogenase-like protein | |
| 700356675H1 | g1235717 | 32 | 1 | gb105pln | Arabidopsis thaliana mRNA for calcium-dependent protein kinase (CDPK), | |
| | | | | | complete cds. | |
| 700352890H1 | g1895083 | 66 | −80 | gb105pln | Zea mays golgi associated protein se-wap41 mRNA, complete cds. | |
| 700353206H1 | g473878 | 22 | −3 | gb105eukp | calnexin homolog | |
| 700352808H1 | g1335965 | 45 | −58 | gb105pln | Zea mays acetyl CoA carboxylase mRNA, partial cds. | |
| 700352973H1 | g473360 | 26 | 0 | gb105allp | Phl pI allergen | |
| 700354902H1 | g168400 | 29 | −27 | gb105pln | Zea mays auxin-binding protein | |
| 700354405H1 | g170781 | 38 | −3 | gb105pln | Wheat ubiquitin carrier protein (E2) mRNA, complete cds. | |
| 700356174H1 | g2623961 | 30 | −8 | gb105pln | A. graveolens mRNA for isocitrate dehydrogenase (NADP+). | |
| 700354363H1 | g1498358 | 56 | −46 | gb105pln | Nicotiana tabacum actin (Tob103) gene, partial cds. | |
| 700355288H1 | g1616658 | 36 | −17 | gb105pln | Triticum aestivum adenylosuccinate synthetase mRNA, partial cds. | |
| 700383076H1 | g414702 | 16 | 4 | gb105pln | H. lanatus mRNA for allergen Hol-lI. | |
| 700354841H1 | g577818 | 27 | −20 | gb105pln | Z. mays gene for H2B histone (gH2B4). | |
| 700352875H1 | g2245136 | 19 | −12 | gb105eukp | trehalose-6-phosphate synthase homolog | |
| 700353723H1 | g482936 | 42 | 7 | gb105eukp | Glycolytic enzyme; pyruvate kinase; plastid isozyme | |
| 700353987H1 | g22418 | 53 | 6 | gb105pln | Z. mays gene for polygalacturonase (clone 17). | |
| 700355001H1 | g2760168 | 19 | 13 | gb105pln | Arabidopsis thaliana genomic DNA, chromosome 5, P1 clone: MEE6, complete | |
| | | | | | sequence. | |
| 700355823H1 | g2347186 | 18 | −15 | gb105pln | Arabidopsis thaliana chromosome II BAC T09D09 genomic sequence, complete | |
| | | | | | sequence. | |
| 700355765H1 | g218157 | 83 | 4 | gb105eukp | cytoplasmic aldolase | |
| 700355865H1 | g2266661 | 26 | −35 | gb105pln | Hordeum vulgare mRNA for 14-3-3 protein (Hv1433c). | |
| 700352923H1 | g287829 | 100 | −57 | gb105pln | Z. mays gene for polygalacturonase. | |
| 700355095H1 | g173231 | 24 | 3 | gb105pln | Yeast (Saccharomyces cerevisiae) ribosomal 5S RNA-binding protein (YL3) gene, 5′ cds. | |
| 700356573H1 | g1814402 | 45 | −33 | gb105pln | Mesembryanthemum crystallinum methionine synthase (MetE) mRNA, complete cds. | |
| 700353246H1 | g1209700 | 28 | −52 | gb105pln | Zea mays ribosomal protein L12 mRNA, complete cds. | |
| 700352674H1 | g1431716 | 42 | −28 | gb105pln | P. agilia beta-1 tubulin (beta-1-tub) gene, complete cds. | |
| 700353286H1 | g1052816 | 22 | −25 | gb105pln | T. aestivum mRNA for profilin (clone TaPRO1). | |
| 700356465H1 | g168549 | 77 | −30 | gb105pln | Zea mays putative anthranilate synthase component II homolog mRNA, partial cds. | |
| 700355789H1 | g553080 | 37 | 7 | gb105allp | chloroplast EF-G | |
| 700383087H1 | g1276967 | 31 | −7 | gb105eukp | putative ribosomal protein | |
| 700355285H1 | g394755 | 11 | 2 | gb105eukp | CF(o)II ATP synthase subunit 9 | |
| 700382111H1 | g168482 | 74 | −82 | gb105pln | Corn starch branching enzyme II mRNA, complete cds. | |
| 700353109H1 | g311906 | 42 | −23 | gb105pln | N. tabacum mRNA for ras-related GTP-binding protein. | |
| 700356246H1 | g804656 | 38 | −01 | gb105eukp | BGQ60; beta-glucosidase | |
| 700356427H1 | g1556428 | 37 | −14 | gb105pln | N. tabacum mRNA for citrate synthase. | |
| 700282577H2 | g1658173 | 42 | −26 | gb105eukp | otaA; ornithine transaminase; EC 2.6.1.13 | |
| 700355860H1 | g2641618 | 33 | −41 | gb105pln | Zea mays ubiquitin-conjugating enzyme protein E2 (ubc7) mRNA, complete cds. | |
| 700382608H1 | g22456 | 96 | −89 | gb105pln | Z. mays pollen specific mRNA C-terminal (clone 3C12). | |
| 700383075H1 | g886470 | 37 | −23 | gb105pln | C. roseus MetE mRNA for methionine synthase. | |
| 700355226H1 | g1246115 | 16 | 4 | gb105pln | major allergen Pha a 1 [Phalaris aquatica = canary grass, pollen, mRNA Partial, | |
| | | | | | 1088 nt]. | |
| 700353573H1 | g293901 | 92 | −39 | gb105pln | Zea mays Zea mI gene, complete cds. | |
| 700354909H1 | g288611 | 94 | −84 | gb105pln | Z. mays PG gene for polygalacturonase. | |
| 700356244H1 | g2160155 | 27 | −2 | gb105pln | Sequence of BAC F21M12 from Arabidopsis thaliana chromosome 1, complete | |
| | | | | | sequence. | |
| 700353135H1 | g2282034 | 20 | −2 | gb105allp | p41-Arc | |
| 700282410H2 | g313137 | 24 | −6 | gb105pln | Z. mays ZmPRO1 mRNA for profilin 1. | |
| 700382250H1 | g1263911 | 15 | −0 | gb105eukp | vacuolar H(+)-ATPase | |
| 700383140H1 | g2244991 | 28 | −21 | gb105pln | Arabidopsis thaliana DNA chromosome 4, ESSA I contig fragment No. 6. | |
| 700356487H1 | g1854375 | 87 | −55 | gb105pln | Saccharum officinarum mRNA for Sucrose-Phosphate Synthase, partial cds. | |
| 700352694H1 | g287829 | 74 | −66 | gb105pln | Z. mays gene for polygalacturonase. | |
| 700282577H2 | g790956 | 44 | −27 | gb105eukp | Dana\Oat; ornithine aminotransferase; EC 2.6.1.13 | |
| 700355953H1 | g975887 | 41 | −31 | gb105pln | Mesembryanthemum crystallinum myo-inositol-1-phosphate synthase mRNA, | |
| | | | | | complete cds. | |
| 700356156H1 | g854625 | 16 | −41 | gb105pln | T. aestivum mRNA for peptidylprolyl cis-trans isomerase, FK506. | |
| 700354304H1 | g763328 | 22 | −13 | gb105eukp | unknown | |
| 700382749H1 | g414703 | 27 | 3 | gb105eukp | allergen Hol-lI | |
| 700354808H1 | g1679658 | 9 | 6 | gb105eukp | gamma glutamyl hydrolase | |
| 700356213H1 | g313139 | 43 | −8 | gb105pln | Z. mays ZmPRO2 mRNA for profilin 2. | |
| 700356752H1 | g2341042 | 34 | 2 | gb105eukp | F19P19.26; F19P19.26 | |
| 700355953H1 | g1513227 | 40 | −30 | gb105pln | Brassica napus myo-inositol 1-phosphate synthase mRNA, complete cds. | |
| 700382620H1 | g166966 | 20 | −0 | gb105pln | Hordeum vulgare acyl carrier protein I (Acl1) gene, complete cds. | |
| 700353225H1 | g577611 | 85 | −77 | gb105pln | Zea mays CRT1 gene for calcium-binding protein. | |
| 700356492H1 | g1519248 | 50 | −24 | gb105pln | Oryza sativa GF14-b protein mRNa, complete cds. | |
| 700383004H1 | g2622710 | 9 | 3 | gb105allp | histidinol-phosphate aminotransferase | |
| 700352631H1 | g436959 | 42 | −1 | gb105eukp | 20 kDa cyclophilin precursor | |
| 700354823H1 | g1620985 | 9 | 15 | gb105pln | N. plumbaginifolia mRNA for 40S ribosomal protein S17. | |
| 700355179H1 | g747848 | 42 | −24 | gb105pln | H. vulgare (Gerbel) mRNA for adenosine triphosphatase. | |
| 700355765H1 | g22144 | 76 | 3 | gb105pln | Maize anaerobically regulated gene for fructose bisphosphate aldolase (EC 4.1.2.13). | |
| 700355817H1 | g2464946 | 43 | −20 | gb105eukp | purple acid phosphatase | |
| 700356794H1 | g1216483 | 36 | −3 | gb105pln | Arabidopsis thaliana dual specificity kinase 1 (ADK1) mRNA, complete cds. | |
| 700383159H1 | g287830 | 8 | 6 | gb105eukp | polygalacturonase | |
| 700354551H1 | g313137 | 54 | −32 | gb105pln | Z. mays ZmPRO1 mRNA for profilin 1. | |
| 700356448H1 | g1572719 | 42 | −4 | gb105eukp | PAST-1 | |
| 700356775H1 | g20807 | 44 | −7 | gb105pln | P. sativum mRNA for MAP kinase homlogue. | |
| 700382669H1 | g2460250 | 30 | −49 | gb105pln | Hordeum vulgare ferrochelatase (hemH) gene, complete cds. | |
| 700355489H1 | g438004 | 45 | −34 | gb105pln | F. pringlei mRNA for T subunit of glycine decarboxylase multi-enzyme complex. | |
| 700357431H1 | g19101 | 38 | −26 | gb105pln | H. vulgare mRNA for ribosomal protein L17-1. | |
| 700357479H1 | g2809480 | 46 | −11 | gb105pln | Oryza sativa calmodulin (CaM2) mRNA, complete cds. | |
| 700282482H2 | g1574722 | 8 | 6 | gb105allp | DNA gyrase, subunit A (gyrA) | |
| 700352644H1 | g1498391 | 66 | −60 | gb105pln | Zea mays actin (Maz 65) gene, partial cds. | |
| 700353153H1 | g780371 | 43 | −35 | gb105pln | Oryza sativa enolase mRNA, complete cds. | |
| 700382528H1 | g886402 | 22 | −5 | gb105pln | Oryza sativa MADS-box protrein (MADS4) mRNA, complete cds. | |
| 700353002H1 | g2559006 | 14 | 3 | gb105eukp | BA1; beta-amylase; EC 3.2.1.2 | |
| 700282513H2 | g1402638 | 8 | −3 | gb105allp | PrfB | |
| 700355961H1 | g409569 | 35 | −16 | gb105pln | Eleusine coracana 5S ribosomal RNA gene, complete sequence. | |
| 700382373H1 | g1531764 | 11 | 12 | gb105pln | D. stramonium mRNA for S-adenosylmethionine decarboxylase. | |
| 700354921H1 | g1122443 | 11 | 8 | gb105allp | receptor kinase-like protein | |
| 700357160H1 | g16446 | 9 | 7 | gb105pln | A. thaliana gene for photosystem II 10 kDa polypeptide. | |
| 700354276H1 | g1685051 | 4 | 7 | gb105allp | CD97 | |
| 700355306H1 | g1173556 | 31 | −35 | gb105pln | Oryza sativa Ory s 1 mRNA, complete cds. | |
| 700355765H1 | g168419 | 76 | 3 | gb105pln | Maize (Z. mays) aldolase mRNA, complete cds. | |
| 700354333H1 | g2829135 | 38 | −24 | gb105eukp | PXN-2; peroxidoxin-2 | |
| 700383057H1 | g171389 | 15 | 9 | gb105pln | Yeast (Saccaromyces cerevisiae) dihydrolipoamide dehydrogenase mRNA, complete cds. | |
| 700352962H1 | g414908 | 33 | −5 | gb105eukp | pollen allergen Phl pI | |
| 700357161H1 | g1906829 | 13 | −5 | gb105pln | A. thaliana hsp88.1 gene. | |
| 700352941H1 | g1928959 | 38 | −7 | gb105pln | Actinidia deliciosa pyrroline-5-carboxylate synthetase mRNA, complete cds. | |
| 700356029H1 | g2088826 | 13 | 5 | gb105eukp | R08F11.1 | |
| 700353608H1 | g1381138 | 24 | −10 | gb105eukp | cul-3; CUL-3 | |
| 700353616H1 | g313135 | 98 | −96 | gb105pln | Z. mays mRNA for porin. | |
| 700356609H1 | g2529657 | 24 | 12 | gb105pln | Arabidopsis thaliana chromosome II BAC T30B22 genomic sequence, complete | |
| | | | | | sequence. | |
| 700382427H1 | g34313 | 30 | 7 | gb105allp | lactate dehydrogenase-A | |
| 700356667H1 | g2687430 | 38 | −17 | gb105pln | Acorus gramineus large subunit 26S ribosomal RNA gene, partial sequence. | |
| 700383123H1 | g2154727 | 43 | −54 | gb105pln | C. dactylon mRNA for profilin 2. | |
| 700382474H1 | g288611 | 12 | 16 | gb105pln | Z. mays PG gene for polygalacturonase. | |
| 700354387H1 | g170556 | 14 | −4 | gb105eukp | thioesterase | |
| 700353027H1 | g998429 | 67 | −30 | gb105pln | GRF1 = general regulatory factor [Zea mays, XL80, Genomic, 5348 nt]. | |
| 700356964H1 | g1263170 | 42 | 4 | gb105eukp | GTP-binding protein | |
| 700353202H1 | g596077 | 79 | −78 | gb105pln | Zea mays thiamine biosynthetic enzyme (thil-1) mRNA, complete cds. | |
| 700352909H1 | g169843 | 90 | −52 | gb105pln | Saccarum hybrid phosphoenolpyruvate carboxylase (SCPEPCD1) gene, complete cds. | |
| 700383007H1 | g1495272 | 15 | 7 | gb105pln | A. thaliana mRNA for sugar transporter. | |
| 700382920H1 | g22427 | 26 | −58 | gb105pln | Z. mays gene for polygalacturonase (clone 65). | |
| 700382460H1 | g1872521 | 20 | −5 | gb105eukp | LSD1; negative regulator of cell death; zinc-finger protein Lsd1 | |
| 700382034H1 | g642130 | 23 | −11 | gb105eukp | AME1; protein kinase | |
| 700356867H1 | g2511530 | 65 | −54 | gb105pln | Eleusine indica alpha tubulin 1 (TUA1) mRNA, complete cds. | |
| 700382272H1 | g22487 | 38 | −70 | gb105pln | Maize gene for sucrose synthase. | |
| 700355939H1 | g2623304 | 9 | 8 | gb105eukp | T20B5.10; similar to Medicago nodulin N21 | |
| 700382009H1 | g2160155 | 24 | −10 | gb105pln | Sequence of BAC F21M12 from Arabidopsis thaliana chromosome 1, complete | |
| | | | | | sequence. | |
| 700382486H1 | g2341060 | 73 | −78 | gb105pln | Zea mays translational initiation factor eIF-4A (tif-4A3) mRNA, complete cds. | |
| 700354655H1 | g2264305 | 18 | 6 | gb105pln | Arabidopsis thaliana genomic DNA, chromosome 5, P1 clone: MBK23, complete | |
| | | | | | sequence. | |
| 700382491H1 | g313139 | 28 | −57 | gb105pln | Z. mays ZmPRO2 mRNA for profilin 2. | |
| 700355217H1 | g288611 | 40 | −13 | gb105pln | Z. mays PG gene for polygalacturonase. | |
| 700382266H1 | g1255684 | 37 | −41 | gb105pln | Rice mRNA for aspartic protease, complete cds. | |
| 700353715H1 | g1052566 | 17 | −2 | gb105pln | C. annuum mRNA for PFTF (pastid fusion and/or translation factor. | |
| 700356794H1 | g1103321 | 29 | 2 | gb105pln | A. thaliana CKI3 mRNA for casein kinase I. | |
| 700382917H1 | g758354 | 37 | 14 | gb105pln | Z. mays mRNA for plasma membrane H+ ATPase. | |
| 700282529H2 | g432606 | 43 | −38 | gb105pln | ric1 = ras-related GTP binding protein possessing GTPase activity [Oryza sativa = rice, | |
| | | | | | Yamohoushi, callus, mRNA, 955 nt]. | |
| 700355634H1 | g1771781 | 17 | 0 | gb105pln | T. aestivum mRNA for beta-amylase. | |
| 700356809H1 | g168419 | 52 | −19 | gb105pln | Maize (Z. mays) aldolase mRNA, complete cds. | |
| 700353251H1 | g1167835 | 24 | −6 | gb105pln | H. lanatus mRNA for Hol lI protein. | |
| 700353621H1 | g2632128 | 91 | −86 | gb105pln | Zea mays mRNA for poly(ADP-ribose) polymerase (3211bp). | |
| 700355950H1 | g2583128 | 18 | 4 | gb105allp | hypothetical protein | |
| 700382843H1 | g1209258 | 14 | −0 | gb105eukp | protease inhibitor II | |
| 700382579H1 | g498747 | 9 | 2 | gb105eukp | Pir7b | |
| 700355735H1 | g2431768 | 27 | −28 | gb105pln | Zea mays acidic ribosomal protein P1a (rpp1a) mRNA, complete cds. | |
| 700352952H1 | g287829 | 100 | −87 | gb105pln | Z. mays gene for polygalacturonase. | |
| 700353586H1 | g2827544 | 29 | 5 | gb105eukp | T12H17.60; HSP associated protein like | |
| 700353657H1 | g288373 | 38 | −50 | gb105pln | Z. mays PGg6 gene for polygalacturonase. | |
| 700382044H1 | g22421 | 92 | −84 | gb105pln | Z. mays mRNA for polygalacturonase (clone PG3). | |
| 700383072H1 | g2245032 | 12 | −5 | gb105eukp | gibberellin oxidase homolog | |
| 700355179H1 | g2267582 | 26 | −11 | gb105pln | Gossypium hirsutum vacuolar H+-ATPase subunit E mRNA, complete cds. | |
| 700382768H1 | g414907 | 33 | −4 | gb105pln | P. pratense mRNA for pollen allergen PhlpI. | |
| 700354412H1 | g288378 | 56 | −17 | gb105pln | Z. mays gene PG for polygalacturonase. | |
| 700356163H1 | g1015926 | 7 | 2 | gb105eukp | SPAC22F3.01; unknown | |
| 700353849H1 | g169539 | 28 | −12 | gb105pln | Potato pyrophosphate-fructose 6-phosphate 1-phosphotransferase (PFP) beta-subunit | |
| | | | | | mRNA, complete cds. | |
| 700356076H1 | g168557 | 22 | 14 | gb105pln | Zea mays putative GTP-binding protein homolog mRNA, partial cds. | |
| 700355659H1 | g22416 | 21 | −12 | gb105pln | Z. mays mRNA for polygalacturonase (clone PG1). | |
| 700382256H1 | g1100222 | 50 | −29 | gb105pln | Pinus sylvestris chloroplast NAD+-dependent glyceraldehyde-3-phosphate dehydrogenase | |
| | | | | | mRNA, complete cds. | |
| 700354786H1 | g473360 | 19 | 2 | gb105allp | Phl pI allergen | |
| 700282418H2 | g168652 | 49 | −86 | gb105pln | Maize amyloplast-specific transit protein (waxy; wc+ locus), complete cds. | |
| 700352655H1 | g902583 | 63 | −69 | gb105pln | Zea mays clone MubG1 ubiquitin gene, complete cds. | |
| 700382946H1 | g499011 | 43 | −34 | gb105pln | S. vulgare SoAc1 mRNA. | |
| 700354224H1 | g22420 | 63 | 5 | gb105pln | Z. mays mRNA for polygalacturonase (clone PG2). | |
| 700355896H1 | g414702 | 29 | −11 | gb105pln | H. lanatus mRNA for allergen Hol-lI. | |
| 700356287H1 | g22458 | 63 | −82 | gb105pln | Z. mays pollen specific mRNA C-terminal (clone 4H7). | |
| 700352637H1 | g313761 | 71 | −73 | gb105pln | Z. mays hsp 70-3 gene for heat shock protein 70. | |
| 700353068H1 | g902583 | 64 | −48 | gb105pln | Zea mays clone MubG1 ubiquitin gene, complete cds. | |
| 700382320H1 | g170050 | 44 | −37 | gb105pln | Soybean pyruvate kinase mRNA, complete cds. | |
| 700352603H1 | g248336 | 77 | −69 | gb105pln | polyubiquitin [maize, Genomic, 3841 nt]. | |
| 700282422H2 | g19495 | 18 | 10 | gb105pln | L. perenne RNA for Lol pI gene. | |
| 700355193H1 | g2113828 | 32 | −22 | gb105pln | Yeast (Candida albicans) TOP2 gene. | |
| 700282461H2 | g21953 | 16 | 3 | gb105allp | beta-glucosidase | |
| 700355732H1 | g2224899 | 28 | −5 | gb105eukp | PcMYB1; DNA-binding protein PcMYB1 | |
| 700382477H1 | g433664 | 13 | 14 | gb105pln | A. thaliana for Sec61 beta-subunit homolog. | |
| 700354316H1 | g2462759 | 4 | 6 | gb105allp | Putative Cytochrome B5 | |
| 700355734H1 | g248338 | 77 | −34 | gb105pln | polyuibiquitin [maize, Genomic, 3439 nt]. | |
| 700356907H1 | g303856 | 71 | −23 | gb105pln | Rice mRNA for ubiquitin protein fused to a ribosomal protein, complete cds. | |
| 700382348H1 | g1800218 | 89 | −75 | gb105pln | Sorghum bicolor phytochome C (PHYC) mRNA, complete cds. | |
| 700382317H1 | g431259 | 20 | 3 | gb105pln | Arabidopsis thaliana 16 kDa ubiquitin conjugating enzyme (UBC1) gene, complete cds. | |
| 700353091H1 | g166626 | 31 | −38 | gb105pln | A. thaliana 57 Kd nucleotide-binding subunit of the vacuolar ATPase mRNA, | |
| | | | | | complete cds. | |
| 700382122H1 | g558651 | 44 | −31 | gb105pln | T. asestivum VDAC3 mRNA for voltage dependent anion channel. | |
| 700353164H1 | g168406 | 25 | 2 | gb105pln | Z. mays alcohol dehydrogenase (ADH-1 C-m allele) gene, complete cds. | |
| 700382807H1 | g21629 | 48 | −52 | gb105pln | Sorghum vulgare mRNA for phosphoenolpyruvate carboxylase (PEPC). | |
| 700383125H1 | g1673433 | 24 | −5 | gb105allp | translocon-associated protein delta subunit precursor | |
| 700354249H1 | g1724101 | 76 | −9 | gb105pln | Mesembryanthemum crystallinum S-adenosyl-L-homocystein hydrolase mRNA, | |
| | | | | | complete cds. | |
| 700382687H1 | g1276967 | 17 | −3 | gb105eukp | putative ribosomal protein | |
| 700382649H1 | g1654385 | 7 | 7 | gb105allp | vacuolar-type H(+) ATPase 115 kDa subunit | |
| 700355313H1 | g602605 | 37 | −75 | gb105pln | Zea mays tandem genes for alpha1-tubulin and alpha2-tubulin. | |
| 700382329H1 | g288366 | 96 | −83 | gb105pln | Z. mays PGg14 gene for polygalacturonase. | |
| 700382033H1 | g2104538 | 22 | 4 | gb105eukp | T10M13.16; T10M13.16 | |
| 700355971H1 | g218112 | 17 | −33 | gb105pln | Rice mRNA for ribosomal protein L41 (340 gene), partial sequence. | |
| 700354909H1 | g288378 | 71 | −63 | gb105pln | Z. mays gene PG for polygalacturonase. | |
| 700355509H1 | g644492 | 51 | −78 | gb105pln | Corn elongation factor 1alpha gene, complete cds. | |
| 700382134H1 | g1388076 | 25 | −8 | gb105eukp | TRX3; thioredoxin h | |
| 700355281H1 | g168460 | 62 | −0 | gb105pln | Zea mays cyclophilin (Cyp) mRNA, complete cds. | |
| 700352636H1 | g2209343 | 8 | 4 | gb105allp | copper transport protein Atox1 | |
| 700382435H1 | g288378 | 81 | −5 | gb105pln | Z. mays gene PG for polygalacturonase. | |
| 700355947H1 | g1173557 | 4 | 8 | gb105allp | Ory s 1 | |
| 700354690H1 | g22456 | 63 | −49 | gb105pln | Z. mays pollen specific mRNA C-terminal (clone 3C12). | |
| 700357430H1 | g551068 | 32 | 2 | gb105eukp | ESI3; unknown; salt-stress induced hydrophobic peptide | |
| 700357164H1 | g1209258 | 19 | −2 | gb105eukp | protease inhibitor II | |
| 700357483H1 | g22425 | 95 | −29 | gb105pln | Z. mays gene for polygalacturonase (clone 47). | |
| 700382935H1 | g1420003 | 10 | 7 | gb105eukp | HRP1 | |
| 700383136H1 | g2306767 | 27 | −5 | gb105pln | Triticum aestivum eIF-2 beta subunit mRNA, complete cds. | |
| 700357450H1 | g1184773 | 35 | −63 | gb105pln | Zea mays glyceraldehyde-3-phosphate dehydrogenase GAPC3 (gpc3) mRNA, | |
| | | | | | complete cds. | |
| 700382822H1 | g288373 | 37 | −32 | gb105pln | Z. mays PGg6 gene for polygalacturonase. | |
| 700353442H1 | g1375088 | 23 | −23 | gb105eukp | DYS 1; NAD-dependent aminobutyl-transferring from spermidine to eIF5A; | |
| | | | | | deoxyhypusine synthase | |
| 700382825H1 | g466572 | 39 | −28 | gb105pln | Tobacco mRNA for RNA helicase like protein DB10. | |
| 700353221H1 | g1173556 | 16 | −4 | gb105pln | Oryza sativa Ory s 1 mRNA, complete cds. | |
| 700382845H1 | g495728 | 21 | −13 | gb105pln | Arabidopsis thaliana small Ras-like GTP-binding protein (Ran-1) mRNA, complete cds. | |
| 700354383H1 | g2582639 | 16 | −6 | gb105eukp | hnRNP-like protein | |
| 700355663H1 | g217973 | 20 | −5 | gb105pln | Zea mays gene for triosephosphate isomerase, complete cds. | |
| 700355030H1 | g483546 | 14 | 5 | gb105pln | R. communis gene for pyrophosphate-dependent phosphofructokinase alpha subunit. | |
| 700353945H1 | g508974 | 33 | −13 | gb105pln | Triticum aestivum Chinese spring protein disulfide isomerase (PDI) mRNA, | |
| | | | | | complete cds. | |
| 700354926H1 | g575730 | 21 | −10 | gb105pln | Z. mays mRNA for transmembrane protein. | |
| 700356667H1 | g169818 | 51 | −31 | gb105pln | Rice 25S ribosomal RNA gene. | |
| 700353950H1 | g287829 | 42 | −44 | gb105pln | Z. mays gene for polygalacturonase. | |
| 700355014H1 | g974781 | 42 | −29 | gb105pln | C. blumei kinetoplast met gene for cobalamine-independent methionine synthase. | |
| 700382560H1 | g1184773 | 41 | −27 | gb105pln | Zea mays glyceraldehyde-3-phosphate dehydrogenase GAPC3 (gpc3) mRNA, | |
| | | | | | complete cds. | |
| 700356133H1 | g2827618 | 16 | 6 | gb105pln | Arabidopsis thaliana DNA chromosome 4, BAC clone F10N7 (ESSAII project). | |
| 700382142H1 | g556685 | 52 | −34 | gb105pln | Z. mays mRNA for ADP-ribosylation factor. | |
| 700382283H1 | g403230 | 11 | 7 | gb105allp | ara-5; GTP-binding protein | |
| 700354955H1 | g971853 | 19 | 6 | gb105allp | expressed sequence tag; human homolog | |
| 700352908H1 | g971283 | 21 | 5 | gb105pln | Rice mRNA for ribosomal protein S31, complete cds. | |
| 700356609H1 | g606969 | 37 | −1 | gb105pln | Arabidopsis thaliana cytoplasmic ribosomal protein L18 mRNA, complete cds. | |
| 700357406H1 | g2276367 | 59 | 5 | gb105allp | 60s ribosomal protein | |
| 700382345H1 | g488712 | 37 | −4 | gb105eukp | cr7; ubiquinol-cytochrome c reductase; EC 1.10.2.2 | |
| 700353106H1 | g398607 | 16 | 12 | gb105pln | A. thaliana mRNA for elongation factor 1 beta. | |
| 700354675H1 | g219128 | 26 | −12 | gb105eukp | A_IG002N01.3 | |
| 700356125H1 | g170783 | 31 | −36 | gb105pln | T. aestivum ubiquitin carrier protein mRNA. | |
| 700282414H2 | g939932 | 13 | 7 | gb105allp | allergen Lol pI I | |
| 700356867H1 | g602605 | 43 | −73 | gb105pln | Zea mays tandem genes for alpha1-tubulin and alpha2-tubulin. | |
| 700382242H1 | g1370193 | 15 | 11 | gb105pln | L. japonicus mRNA for small GTP-binding protein, RAB8C. | |
| 700354441H1 | g185949 | 31 | −19 | gb105allp | Ig nonfunctional kappa-chain (C-region) | |
| 700355159H1 | g22425 | 50 | −58 | gb105pln | Z. mays gene for polygalacturonase (clone 47). | |
| 700353253H1 | g1216483 | 21 | −2 | gb105pln | Arabidopsis thaliana dual specificity kinase 1 (ADK1) mRNA, complete cds. | |
| 700383190H1 | g886400 | 51 | −38 | gb105pln | Oryza sativa MADS-box protein (MADS2) mRNA, complete cds. | |
| 700382614H1 | g1929057 | 21 | 1 | gb105eukp | longest ORF (1) | |
| 700282520H2 | g2656030 | 34 | −11 | gb105pln | Arabidopsis thaliana genomic DNA, chromosome 5, P1 clone: MUL8. | |
| 700354553H1 | g1839244 | 56 | −8 | gb105eukp | EGF receptor like protein | |
| 700355519H1 | g20321 | 27 | −29 | gb105pln | Oryza sativa RAc1 mRNA for actin. | |
| 700357441H1 | g414702 | 22 | −3 | gb105pln | H. lanatus mRNA for allergen Hol-lI. | |
| 700354992H1 | g473359 | 20 | −15 | gb105pln | P. pratense mRNA for Phl pI allergen. | |
| 700355337H1 | g777757 | 52 | −44 | gb105pln | Saccharum hybrid (clone SCUBI561) polyubiquitin mRNA, complete cds. | |
| 700382764H1 | g460988 | 59 | −56 | gb105pln | O. sativa (Arborio) Beta Tubulin mRNA, clone OSTB-34. | |
| 700383083H1 | g218177 | 26 | −6 | gb105pln | Rice mRNA for ribosomnal protein L356 (NH77 gene), partial sequence. | |
| 700353047H1 | g167109 | 60 | −18 | gb105pln | Hordeum vulgare vacuolar ATPase B subunit mRNA, complete cds. | |
| 700355862H1 | g2154729 | 46 | −35 | gb105pln | C. dactylon mRNA for profilin 1. | |
| 700383160H1 | g168460 | 60 | −63 | gb105pln | Zea mays cyclophilin (CyP) mRNA, complete cds. | |
| 700382633H1 | g5056 | 19 | 5 | gb105pln | Yeast (Saccharomyces pombe) rpgL29 gene for ribosomal protein L29. | |
| 700356112H1 | g2529339 | 30 | −11 | gb105pln | Zea maize AGAMOUS homologue mRNA, complete cds. | |
| 700353960H1 | g1053056 | 37 | −38 | gb105pln | Triticum aestivum histone H3 gene, partial cds, clone W1. | |
| 700353239H1 | g1932927 | 12 | −5 | gb105eukp | putative pectin methylesterase | |
| 700352776H1 | g248336 | 64 | −58 | gb105pln | polyubiquitin [maize, Genomic, 3841 nt]. | |
| 700355951H1 | g473359 | 19 | −1 | gb105pln | P. pratense mRNA for Phl pI allergen. | |
| 700353140H1 | g2804257 | 16 | 10 | gb105pln | Arabidopsis thaliana DNA for phosphoglycerate dehydrogenase, complete cds. | |
| 700356233H1 | g312180 | 61 | −56 | gb105pln | Z. mays GapC4 gene. | |
| 700356893H1 | g886470 | 25 | −1 | gb105pln | C. roseus MetE mRNA for methionine synthase. | |
| 700353635H1 | g1066162 | 48 | −14 | gb105pln | B. napus mRNA for glyoxysomal beta-ketoacyl-thiolase precursor. | |
| 700383116H1 | g2494106 | 15 | 7 | gb105pln | Arabidopsis thaliana chromosome 1 YAC yUP8H12R, complete sequence. | |
| 700355876H1 | g1655423 | 35 | −25 | gb105pln | Arabidopsis thaliana mRNA for GDP dissociation inhibitor, complete cds. | |
| 700382501H1 | g602605 | 56 | −55 | gb105pln | Zea mays tandem genes for alpha1-tubulin and alpha2-tubulin. | |
| 700356208H1 | g2415701 | 51 | −12 | gb105pln | P. pratense mRNA for profilin 4. | |
| 700353546H1 | g2326873 | 40 | −29 | gb105pln | S. tuberosum mRNA for plastidic ATP/ADP-transporter. | |
| 700382961H1 | g473976 | 17 | −38 | gb105pln | Rice mRNA, partial homologous to elongation factor 1-alpha gene. | |
| 700382972H1 | g2415699 | 20 | −14 | gb105pln | P. pratense mRNA for profilin 3. | |
| 700354528H1 | g2149639 | 42 | −6 | gb105pln | Arabidopsis thaliana Argonaute protein (AGO1) mRNA, complete cds. | |
| 700382231H1 | g1304412 | 25 | −9 | gb105pln | Arabidopsis thaliana Rac-like protein (ARAC3) gene, complete cds. | |
| 700383076H1 | g168315 | 17 | 5 | gb105pln | L. perenne pollen allergen (Lol pI ) gene, complete cds. | |
| 700355217H1 | g288378 | 22 | 0 | gb105pln | Z. mays gene PG for polygalacturonase. | |
| 700382924H1 | g1418317 | 12 | 2 | gb105eukp | CER1-like | |
| 700354392H1 | g22425 | 80 | −72 | gb105pln | Z. mays gene for polygalacturonase (clone 47). | |
| 700353033H1 | g2459428 | 25 | 3 | gb105eukp | F4P9.23 | |
| 700355689H1 | g22458 | 17 | −43 | gb105pln | Z. mays pollen specific mRNA C-terminal (clone 4H7). | |
| 700355912H1 | g2766449 | 56 | −53 | gb105pln | Sorghum bicolor cytochrome P450 CYP99A1 (CYP99A1) mRNA, partial cds. | |
| 700357409H1 | g498772 | 44 | −34 | gb105pln | Z. mays (cv DH5xDH7) hsp70-4 mRNA for heat shock protein 70. | |
| 700354727H1 | g1762938 | 22 | −6 | gb105pln | Nicotiana tabacum ORF mRNA, complete cds. | |
| 700352772H1 | g968995 | 62 | −44 | gb105pln | Oryza sativa clone glyceraldehyde-3-phosphate dehydrogenase (Gpc) mRNA, | |
| | | | | | complete cds. | |
| 700282516H2 | g1049306 | 13 | −2 | gb105pln | Arabidopsis thaliana actin-2 mRNA, complete cds. | |
| 700355809H1 | g298069 | 12 | 8 | gb105pln | A. thaliana Em protein gene (ATEM1). | |
| 700382436H1 | g488835 | 6 | 5 | gb105allp | ribosomal protein L28 | |
| 700357184H1 | g1041197 | 6 | 8 | gb105eukp | sconB; sulfur metabolite repression control | |
| 700354581H1 | g1568635 | 41 | −2 | gb105eukp | host cell receptor involved in nuclear import of Agrobacterium VirD2 protein; AtKAP | |
| | | | | | alpha | |
| 700354609H1 | g1167835 | 37 | −35 | gb105pln | H. lanatus mRNA for Hol lI protein. | |
| 700353524H1 | g1695716 | 21 | 17 | gb105pln | Arabidopsis thaliana DNA for luminal binding protein, complete cds. | |
| 700356254H1 | g2465560 | 8 | 6 | gb105allp | phosphoribosylaminoimidazole-carboxamide formyltransferase; formyltransferase/IMP | |
| | | | | | cyclohydrolase | |
| 700355961H1 | g20070 | 29 | −18 | gb105pln | O. minuta 5S DNA. | |
| 700352724H1 | g632679 | 10 | 2 | gb105eukp | CDC73; Cdc73p | |
| 700355411H1 | g393449 | 26 | −31 | gb105pln | S. cereale mRNA for beta-amylase. | |
| 700282489H2 | g416256 | 24 | 2 | gb105pln | Rice mRNA for H+-ATPase, partial sequence. | |
| 700355424H1 | g432488 | 39 | −42 | gb105pln | Wheat initiation factor 1A (eIF-1A) mRNA. | |
| 700354434H1 | g644491 | 35 | −34 | gb105pln | Corn mRNA for elongation factor 1A. | |
| 700356479H1 | g886400 | 42 | −14 | gb105pln | Oryza sativa MADS-box protein (MADS2) mRNA, complete cds. | |
| 700356864H1 | g397958 | 81 | −77 | gb105pln | Z. mays mRNA for beta-amylase. | |
| 700357147H1 | g550546 | 36 | −41 | gb105pln | A. thaliana RPL16B gene. | |
| 700382501H1 | g2511530 | 43 | −39 | gb105pln | Eleusine indica alpha tubulin 1 (TUA1) mRNA, complete cds. | |
| 700354394H1 | g2641618 | 57 | −40 | gb105pln | Zea mays ubiquitin-conjugating enzyme protein E2 (ubc7) mRNA, complete cds. | |
| 700353060H1 | g1272684 | 81 | −73 | gb105pln | Z. mays mRNA for acetyl CoA carboxylase (partial). | |
| 700356233H1 | g1184775 | 98 | −62 | gb105pln | Zea mays glyceraldehyde-3-phosphate dehydrogenase GAPC4 (gpc4) mRNA, | |
| | | | | | complete cds. | |
| 700357171H1 | g433043 | 10 | 8 | gb105pln | Zea mays W-22 clone PREM-1E retroelement PREM-1, partial sequence. | |
| 700382901H1 | g299814 | 24 | 1 | gb105pln | tuber-induction gene {3′ region} [Solanum tuberosum = potatoes, cv. Desiree, leaves, | |
| | | | | | mRNA Partial, 950 nt]. | |
| 700357406H1 | g531169 | 67 | 3 | gb105allp | Csa-19 | |
| 700353576H1 | g595816 | 22 | −3 | gb105eukp | mudrA | |
| 700357491H1 | g1051108 | 39 | −6 | gb105pln | A. thaliana mRNA for adenine nucleotide translocase. | |
| 700354511H1 | g1881694 | 73 | −27 | gb105pln | Zea mays phosphoglucomutase mRNA, partial cds. | |
| 700352906H1 | g1438882 | 28 | −13 | gb105pln | Glycine max choline kinase GmCK3p mRNA, partial cds. | |
| 700382102H1 | g348695 | 30 | −34 | gb105pln | Catharanthus roseus heat shock protein 90 mRNa, complete cds. | |
| 700355426H1 | g18936 | 9 | −4 | gb105eukp | BKIN9; protein kinase | |
| 700354852H1 | g474001 | 17 | 10 | gb105pln | Rice mRNA, partial homologous to ribosomal protein L38 gene. | |
| 700383116H1 | g849195 | 8 | 2 | gb105eukp | YDR374C; Ydr374cp | |
| 700353658H1 | g473258 | 51 | −45 | gb105pln | Arabidopsis thaliana Ser/Thr protein phosphatase mRNA, complete cds. | |
| 700356658H1 | g1236977 | 48 | −11 | gb105pln | Arabidopsis thaliana CTP:phosphocholine cytidylyltransferase gene, complete cds. | |
| 700356285H1 | g1322722 | 21 | −7 | gb105eukp | MRF1 | |
| 700353952H1 | g2460180 | 14 | 5 | gb105allp | RNA binding protein | |
| 700383111H1 | g2662077 | 8 | 7 | gb105allp | KIAA0398 | |
| 700356422H1 | g2809482 | 24 | −2 | gb105pln | Oryza sativa calmodulin (CaM1) mRNA, partial cds. | |
| 700382474H1 | g433040 | 16 | 13 | gb105pln | Zea mays W-22 clone PREM-1A retroelement PREM-1, partial sequence. | |
| 700383173H1 | g1136784 | 24 | −21 | gb105pln | Fission Yeast mRNA for elongation factor 1 alpha-B, complete cds. | |
| 700354919H1 | g312178 | 47 | 6 | gb105pln | Z. mays GapC2 gene. | |
| 700354542H1 | g2463334 | 30 | −40 | gb105pln | Oryza sativa mRNA for ribosomal protein S4. | |
| 700352701H1 | g860963 | 52 | −26 | gb105allp | DNA ligase III | |
| 700356463H1 | g1217651 | 68 | 1 | gb105allp | carbonyl reductase (NADPH) | |
| 700355555H1 | g2282583 | 48 | −64 | gb105pln | Zea mays elongation factor 1-alpha (EF1-A) mRNA, complete cds. | |
| 700353045H1 | g929917 | 23 | −20 | gb105pln | Z. mays ABP1 mRNA for actin-depolymerizing factor. | |
| 700354724H1 | g2651294 | 13 | 1 | gb105pln | Arabidopsis thaliana chromosome II BAC T2P4 genomic sequence, complete sequence. | |
| 700352988H1 | g22416 | 98 | −55 | gb105pln | Z. mays mRNA for polygalacturonase (clone PG1). | |
| 700354373H1 | g456717 | 31 | −24 | gb105pln | A. gossypii TEF gene for translation elongation factor 1 alpha (EF-1 alpha). | |
| 700352978H1 | g22342 | 72 | −9 | gb105pln | Maize gene for heat shock protein 70 exon 2 and 3′-UT (hsp70; clone pMON 9502). | |
| 700356210H1 | g2407616 | 26 | −9 | gb105pln | Lycopersicon esculentum glutathione synthetase (GSH2) mRNA, complete cds. | |
| 700354517H1 | g459748 | 26 | 6 | gb105allp | Sec61-complex gamma-subunit | |
| 700382489H1 | g37836 | 51 | −41 | gb105allp | Immunoglobulin heavy chain | |
| 700354581H1 | g2154717 | 34 | 1 | gb105eukp | Kap alpha protein | |
| 700355179H1 | g1143508 | 29 | −25 | gb105pln | M. crystallinum mRNA for vauolar proton ATPase subunit E. | |
| 700382061H1 | g20310 | 12 | 7 | gb105eukp | pollen specific gene | |
| 700382265H1 | g167559 | 48 | −27 | gb105pln | Carrot 70 kd subunit vacular H+-ATPase, complete cds. | |
| 700382767H1 | g2388789 | 30 | −5 | gb105eukp | cytochrome oxidase I | |
| 700354307H1 | g397841 | 55 | −19 | gb105pln | Rice mRNA for ferredoxin-NADP+ reductase, complete cds. | |
| 700353857H1 | g2262153 | 19 | −1 | gb105eukp | T10P11.19; putative chloroplast outer envelope 86-like protein | |
| 700282576H2 | g790956 | 44 | −26 | gb105eukp | Dana\Oat; ornithine aminotransferase; EC 2.6.1.13 | |
| 700355203H1 | g1706958 | 37 | 1 | gb105eukp | celA2; cellulose synthase | |
| 700355305H1 | g599874 | 7 | 8 | gb105allp | NADH-ubiquinone oxidoreductase complex B22 subunit | |
| 700382266H1 | g18903 | 34 | −34 | gb105pln | Barley mRNA for aspartic proteinase. | |
| 700356476H1 | g22418 | 100 | −59 | gb105pln | Z. mays gene for polygalacturonase (clone 17). | |
| 700355054H1 | g479119 | 48 | −24 | gb105pln | F. pringlei fpcpt mRNA for triose phosphate/phosphate translocator. | |
| 700352770H1 | g2270994 | 32 | −13 | gb105eukp | GmPM13; Ca+2-binding EF hand protein | |
| 700282420H2 | g2245108 | 17 | −8 | gb105eukp | EREBP-4 homolog | |
| 700382351H1 | g509769 | 17 | −0 | gb105eukp | seed-specific low molecular weight sulfur-rich protein | |
| 700352962H1 | g473360 | 34 | −5 | gb105eukp | phl pI ; Phl pI allergen | |
| 700352658H1 | g288373 | 98 | −58 | gb105pln | Z. mays PGg6 gene for polygalacturonase. | |
| 700356196H1 | g2275006 | 33 | −18 | gb105pln | Hordeum vulgare mRNA for expressed sequence tag. | |
| 700353642H1 | g304106 | 15 | −4 | gb105pln | Arabidopsis thaliana cytosine-5 methyltransferase mRNA, complete cds. | |
| 700355419H1 | g596079 | 83 | −70 | gb105pln | Zea mays thiamine biosynthetic enzyme (thi1-2) mRNA, complete cds. | |
| 700355887H1 | g169988 | 31 | −41 | gb105pln | Glycine max NADP-specific isocitrate dehydrogenase (idh1) mRNA, 3′ end. | |
| 700382246H1 | g288373 | 65 | −69 | gb105pln | Z. mays PGg6 gene for polygalacturonase. | |
| 700354543H1 | g624675 | 44 | −7 | gb105pln | Citrus maxima citrate synthase (cit) mRNA, nuclear gene encoding mitochondrial | |
| | | | | | protein, complete cds. | |
| 700354490H1 | g1632767 | 12 | −8 | gb105pln | Maize mRNA for calcium dependent protein kinase, complete cds. | |
| 700355456H1 | g1498389 | 35 | −74 | gb105pln | Zea mays actin (Maz81) gene, partial cds. | |
| 700357165H1 | g902583 | 72 | −82 | gb105pln | Zea mays clone MubG1 ubiquitin gene, complete cds. | |
| 700382311H1 | g606065 | 11 | 4 | gb105allp | ORF_f299 | |
| 700354551H1 | g453975 | 37 | 7 | gb105pln | P. pratense mRNA for profilin-Phl p XI. | |
| 700355522H1 | g22421 | 57 | −76 | gb105pln | Z. mays mRNA for polygalacturonase (clone PG3). | |
| 700355242H1 | g1906825 | 10 | 15 | gb105pln | A. thaliana hsp81.2 gene. | |
| 700355322H1 | g2642161 | 8 | 5 | gb105eukp | T5I7.9 | |
| 700355590H1 | g2275006 | 38 | −24 | gb105pln | Hordeum vulgare mRNA for expressed sequence tag. | |
| 700355978H1 | g1718236 | 13 | −1 | gb105eukp | (1,4)-beta-xylan endohydrolase, isoenzyme X-I; EC 3.2.1.8 | |
| 700353234H1 | g2828184 | 16 | 7 | gb105pln | Arabidopsis thaliana genomic DNA, chromosome 5, P1 clone: MSN9, complete | |
| | | | | | sequence. | |
| 700355896H1 | g414907 | 29 | −10 | gb105pln | P. pratense mRNA for pollen allergen PhlpI. | |
| 700382282H1 | g16427 | 16 | −1 | gb105eukp | protease inhibitor II | |
| 700352751H1 | g984704 | 28 | −16 | gb105eukp | SPAC24H6.07; unknown | |
| 700352794H1 | g2160132 | 20 | −1 | gb105pln | Sequence of BAC F19K23 from Arabidopsis thaliana chromosome 1, complete | |
| | | | | | sequence. | |
| 700382129H1 | g2275202 | 9 | 1 | gb105eukp | T08I13.8; acyl-CoA synthetase isolog | |
| 700382715H1 | g786120 | 27 | −1 | gb105allp | ORF1; putative | |
| 700355324H1 | g468554 | 25 | 6 | gb105allp | CCT (chaperonin containing TCP-1) zeta subunit | |
| 700356960H1 | g868225 | 23 | 3 | gb105allp | No definition line found | |
| 700382254H1 | g1132482 | 65 | −57 | gb105pln | Rice mRNA for ADP-ribosylation factor, complete cds. | |
| 700383007H1 | g1495273 | 29 | −10 | gb105eukp | sugar transporter | |
| 700354559H1 | g2245679 | 13 | 2 | gb105eukp | vha44; V-ATPase C subunit | |
| 700352642H1 | g22275 | 83 | −79 | gb105pln | Maize mRNA for ferritin (clone FM1). | |
| 700354821H1 | g565064 | 15 | 15 | gb105pln | Arabidopsis thaliana pyruvate dehydrogenase E1 beta subunit mRNA, complete cds. | |
| 700383137H1 | g1245182 | 19 | 4 | gb105eukp | ST7R; sterol delta-7 reductase | |
| 700356516H1 | g1388080 | 16 | 0 | gb105allp | thioredoxin h | |
| 700382637H1 | g857573 | 25 | −23 | gb105pln | Oryza sativa vacuolar H+-ATPase (vatp-P1) mRNA, complete cds. | |
| 700354623H1 | g603220 | 34 | −33 | gb105pln | Medicago sativa 6-phosphogluconate dehydrogenase mRNA, complete cds. | |
| 700355402H1 | g2252824 | 12 | −3 | gb105eukp | A_IG005I10.1 | |
| 700352906H1 | g1438878 | 23 | −10 | gb105pln | Glycine max choline kinase GmCL1p mRNA, complete cds. | |
| 700355734H1 | g902583 | 91 | −42 | gb105pln | Zea mays clone MubG1 ubiquitin gene, complete cds. | |
| 700382339H1 | g1181330 | 89 | −77 | gb105pln | Z. mays CNX mRNA. | |
| 700354828H1 | g22425 | 50 | −53 | gb105pln | Z. mays gene for polygalacturonase (clone 47). | |
| 700355208H1 | g434345 | 87 | −6 | gb105eukp | S18 ribosomal protein | |
| 700354709H1 | g16509 | 38 | −28 | gb105pln | A. thaliana mRNA for succinate-CoA ligase (GCP-forming). | |
| 700353203H1 | g902789 | 9 | 2 | gb105eukp | ILL1; ILL1 | |
| 700355955H1 | g506832 | 24 | 1 | gb105allp | protein 3 | |
| 700382889H1 | g176779 | 84 | −37 | gb105allp | gamma-2 globin | |
| 700282486H2 | g2749918 | 24 | −12 | gb105pln | Arabidopsis thaliana chromosome I BAC F3I6 genomic sequence, complete sequence. | |
| 700356185H1 | g2702273 | 11 | 2 | gb105eukp | T21L14.15; carrot B2 protein-like | |
| 700356069H1 | g495728 | 59 | 15 | gb105pln | Arabidopsis thaliana small Ras-like GTP-binding protein (Ran-1) mRNA, complete cds. | |
| 700356113H1 | g1246116 | 17 | −3 | gb105eukp | major allergen Pha a 1; major allergen Pha a 1 | |
| 700352827H1 | g2462742 | 24 | −17 | gb105eukp | F8A5.25 | |
| 700356224H1 | g804655 | 20 | −0 | gb105pln | Hordeum vulgare L. beta-glucosidase (BGQ60) gene, complete cds. | |
| 700355463H1 | g22425 | 31 | −61 | gb105pln | Z. mays gene for polygalacturonase (clone 47). | |
| 700356122H1 | g1498628 | 14 | −23 | gb105pln | Arabidopsis thaliana p40 protein homolog mRNA, complete cds. | |
| 700382454H1 | g2827551 | 9 | 5 | gb105eukp | T12H17.130; predicted protein | |
| 700354263H1 | g2460250 | 30 | −48 | gb105pln | Hordeum vulgare ferrochelatase (hemH) gene, complete cds. | |
| 700353945H1 | g625147 | 35 | −32 | gb105pln | Zea mays protein disulfide isomerase (pdi) mRNA, complete cds. | |
| 700356823H1 | g643596 | 38 | −32 | gb105pln | Corn mRNA for cysteine proteinase, clone CCP, complete cds. | |
| 700354410H1 | g1143499 | 31 | −29 | gb105pln | H. vulgare mRNA for ADP-glucose pyrophosphorylase small subunit. | |
| 700352726H1 | g514317 | 32 | −14 | gb105pln | Arabidopsis thaliana RNA polymerase II third largest subunit (RPB35.5A) gene, | |
| | | | | | complete cds. | |
| 700354960H1 | g899115 | 54 | 5 | gb105eukp | polyubiquitin | |
| 700383073H1 | g536114 | 15 | 6 | gb105pln | Yeast (Saccharomyces cerevisiae) chromosome II reading frame ORF YBL072c. | |
| 700354544H1 | g2760084 | 32 | 0 | gb105eukp | G8B7T7; leucine-rich repeat protein | |
| 700353071H1 | g2529657 | 18 | 10 | gb105pln | Arabidopsis thaliana chromosome II BAC T30B22 genomic sequence, complete | |
| | | | | | sequence. | |
| 700357406H1 | g1370289 | 67 | 3 | gb105allp | ribosomal protein L10a | |
| 700382927H1 | g2316015 | 18 | 17 | gb105pln | Arabidopsis thaliana MRP-like ABC transporter mRNA, complete cds. | |
| 700354960H1 | g209603 | 54 | 5 | gb105allp | ubiquitin/relaxin fusion protein | |
| 700356864H1 | g169778 | 15 | 13 | gb105pln | Oryza sativa beta-amylase gene, complete cds. | |
| 700352994H1 | g527680 | 77 | −4 | gb105eukp | ribosomal protein S3 | |
| 700354535H1 | g1209700 | 37 | −48 | gb105pln | Zea mays ribosomal protein L12 mRNA, complete cds. | |
| 700354422H1 | g1911581 | 26 | −30 | gb105pln | Cyn d 1 = major allergen {clone 14c1 and CD1} [Cynodon dactylon = Bermuda grass, | |
| | | | | | pollen, mRNA Partial, 759 nt]. | |
| 700352676H1 | g857573 | 42 | −21 | gb105pln | Oryza sativa vacuolar H+-ATPase (vatp-P1) mRNA, complete cds. | |
| 700355789H1 | g147895 | 32 | 8 | gb105allp | fusA | |
| 700383030H1 | g176780 | 32 | −16 | gb105allp | gamma-1 globin | |
| 700356820H1 | g1498387 | 62 | −68 | gb105pln | Zea mays actin (Maz83) gene, partial cds. | |
| 700352904H1 | g172496 | 27 | −3 | gb105pln | Yeast (Saccharomyces carlsbergensis) ribosomal protein s10 gene. | |
| 700282541H2 | g1657619 | 28 | −4 | gb105eukp | AtG5; putative transmembrane protein; G5p | |
| 700356950H1 | g429009 | 50 | −2 | gb105pln | Rice mRNA for LMW heat shock protein (gene name SS386), partial cds. | |
| 700352804H1 | g507823 | 20 | −6 | gb105eukp | A622 | |
| 700382032H1 | g1834355 | 16 | 7 | gb105allp | hypothetical protein 194 | |
| 700382770H1 | g170201 | 18 | 15 | gb105pln | Nicotiana tabacum mRNA sequence. | |
| 700354921H1 | g2586081 | 12 | 6 | gb105eukp | receptor kinase-like protein | |
| 700352678H1 | g293901 | 30 | −18 | gb105pln | Zea mays Zea mI gene, complete cds. | |
| 700382166H1 | g439585 | 51 | −30 | gb105pln | Hordeum vulgare calreticulin (CRH1) mRNA, partial cds. | |
| 700353523H1 | g22416 | 47 | −41 | gb105pln | Z. mays mRNA for polygalacturonase (clone PG1). | |
| 700356848H1 | g161172 | 8 | 6 | gb105eukp | elongation factor 1-gamma | |
| 700382875H1 | g288611 | 59 | −49 | gb105pln | Z. mays PG gene for polygalacturonase. | |
| 700352829H1 | g2578364 | 13 | 16 | gb105pln | Lycopersicon esculentum mRNA for GTP cyclohydrolase II/3,4-dihydroxy-2-butanone | |
| | | | | | 4-phosphate synthase. | |
| 700382102H1 | g22651 | 71 | −65 | gb105pln | H. vulgare mRNA for GRP94 homologue. | |
| 700354778H1 | g1438881 | 10 | −0 | gb105eukp | GmCK2p; EC 2.7.1.32 | |
| 700355062H1 | g2832611 | 15 | −5 | gb105pln | Arabidopsis thaliana DNA chromosome 4, BAC clone F13C5 (ESSAII project). | |
| 700355969H1 | g303834 | 32 | −27 | gb105pln | Rice mRNA for 21 kd polypeptide, complete cds. | |
| 700354662H1 | g1616627 | 10 | 5 | gb105eukp | smm1; SMM1 protein | |
| 700382232H1 | g1871506 | 24 | −3 | gb105pln | C. dactylon mRNA for calcium-binding pollen allergen. | |
| 700353716H1 | g1872472 | 32 | −22 | gb105pln | Triticum aestivum delta-24-sterol methyltransferase (TA-MT) gene, complete cds. | |
| 700356114H1 | g1155264 | 21 | −13 | gb105pln | Pennisetum ciliare possible apospory-associated protein mRNA, complete cds. | |
| 700356567H1 | g414907 | 46 | −15 | gb105pln | P. pratense mRNA for pollen allergen PhlpI. | |
| 700356773H1 | g1899025 | 37 | −1 | gb105eukp | AtHXK2; hexokinase 2; EC 2.7.1.1 | |
| 700382477H1 | g433665 | 21 | 6 | gb105allp | Sec61 beta-subunit homolog | |
| 700353930H1 | g479119 | 30 | 7 | gb105pln | F. pringlei fpcpt mRNA for triose phosphate/phosphate translocator. | |
| 700355413H1 | g22425 | 100 | −86 | gb105pln | Z. mays gene for polygalacturonase (clone 47). | |
| 700382673H1 | g1389849 | 30 | −16 | gb105eukp | myosin regulatory light chain | |
| 700356273H1 | g896268 | 54 | 4 | gb105allp | protein 5 | |
| 700354457H1 | g287829 | 37 | −54 | gb105pln | Z. mays gene for polygalacturonase. | |
| 700355461H1 | g644491 | 80 | −67 | gb105pln | Corn mRNA for elongation factor 1A. | |
| 700382658H1 | g642120 | 37 | −48 | gb105pln | Oryza sativa small GTP-binding protein (ORRab-2) mRNA, complete cds. | |
| 700352666H1 | g1141785 | 33 | −20 | gb105pln | Yeast (Schizosaccharomyces pombe) QM homolog (SPQM) mRNA, complete cds. | |
| 700352748H1 | g967124 | 31 | −18 | gb105pln | Vigna radiata Rwilcz calcium dependent protein kinase (CDPK) mRNA, complete cds. | |
| 700355723H1 | g289644 | 31 | −15 | gb105eukp | C14B9.7 protein | |
| 700383103H1 | g169820 | 34 | −16 | gb105pln | Oryza sativa triosephosphate isomerase (Rictpi) mRNA, complete cds. | |
| 700354570H1 | g2656029 | 28 | 8 | gb105pln | Arabidopsis thaliana genomic DNA, chromosome 5, P1 clone: MQB2. | |
| 700352920H1 | g179283 | 28 | 7 | gb105allp | PM-Scl autoantigen | |
| 700353902H1 | g1143864 | 27 | −9 | gb105eukp | catalyzes the release of either giberellin or cyanogenic substances from their gluco- | |
| | | | | | conjugates; beta glucosidase; EC 3.2.1.21 | |
| 700355281H1 | g829147 | 58 | −11 | gb105pln | Z. mays gene for cyclophilin. | |
| 700352751H1 | g1177283 | 31 | −18 | gb105eukp | F40F8.10 | |
| 700354472H1 | g176827 | 52 | −39 | gb105allp | beta-2-microglobulin | |
| 700355858H1 | g602605 | 27 | −56 | gb105pln | Zea mays tandem genes for alpha1-tubulin and alpha2-tubulin. | |
| 700354602H1 | g55457 | 22 | −6 | gb105allp | Ypt1 protein (AA 1-205) | |
| 700354224H1 | g22421 | 63 | 5 | gb105pln | Z. mays mRNA for polygalacturonase (clone PG3). | |
| 700355213H1 | g2370460 | 28 | −43 | gb105pln | Sorghum bicolor mRNA for putative glycoprotein. | |
| 700382933H1 | g1574460 | 5 | 6 | gb105allp | aminopeptidase N (pepN) | |
| 700383032H1 | g170752 | 17 | −10 | gb105pln | Wheat (clone p28-62) initiation factor isozyme 4F p28 subunit mRNA, complete cds. | |
| 700354362H1 | g287829 | 38 | −58 | gb105pln | Z. mays gene for polygalacturonase. | |
| 700354250H1 | g1017823 | 14 | 3 | gb105allp | RNA polymerase II subunit | |
| 700382816H1 | g288611 | 39 | −15 | gb105pln | Z. mays PG gene for polygalacturonase. | |
| 700355649H1 | g1668706 | 10 | 4 | gb105eukp | atran2 | |
| 700355847H1 | g1173621 | 43 | −37 | gb105pln | Phalaenopsis sp. ‘hybrid SM9108’ homeobox protein mRNA, complete cds. | |
| 700355921H1 | g984700 | 7 | 6 | gb105eukp | SPAC24H6.03; unknown | |
| 700382902H1 | g1495251 | 23 | −21 | gb105eukp | heat-shock protein | |
| 700353963H1 | g288373 | 78 | −12 | gb105pln | Z. mays PGg6 gene for polygalacturonase. | |
| 700352630H1 | g623585 | 28 | −1 | gb105pln | Nicotiana tabacum SR1 Nt-rab6 mRNA, complete cds. | |
| 700353008H1 | g2224934 | 28 | −5 | gb105pln | Arabidopsis thaliana ethylene-insensitive3 (EIN3) gene, complete cds. | |
| 700383030H1 | g183885 | 33 | −21 | gb105allp | gamma-globin | |
| 700357467H1 | g540534 | 43 | −4 | gb105pln | Rice mRNA for q group of receptor for activated C-kinase, complete cds. | |
| 700354350H1 | g633684 | 21 | −2 | gb105pln | S. tuberosum (Desiree) 8.0 kD subunit of cytochrome C reductase mRNA. | |
| 700355710H1 | g2599103 | 29 | −17 | gb105pln | Dunaliella salina 60S ribosomal protein (DSRP1) mRNA, complete cds. | |
| 700352835H1 | g18259 | 48 | −39 | gb105pln | C. sativus mRNA for cs DnaJ-1. | |
| 700355306H1 | g473359 | 28 | −34 | gb105pln | P. pratense mRNA for Phl pI allergen. | |
| 700353835H1 | g1532072 | 61 | −40 | gb105pln | Z. mays mRNA for S-adenosylmethionine decarboxylase. | |
| 700353183H1 | g288611 | 40 | −61 | gb105pln | Z. mays PG gene for polygalacturonase. | |
| 700355518H1 | g2384676 | 27 | −14 | gb105pln | Arabidopsis thaliana putative potassium transporter AtKT5p (AtKT5) mRNA, | |
| | | | | | partial cds. | |
| 700382749H1 | g473360 | 29 | 2 | gb105allp | Phl pI allergen | |
| 700355856H1 | g161631 | 14 | −0 | gb105eukp | 217 g protein | |
| 700353001H1 | g255568 | 100 | −22 | gb105pln | pollen specific protein [Zea mays = corn, mRNA, 943 nt]. | |
| 700356136H1 | g439616 | 22 | −33 | gb105pln | Soybean G protein alpha subunit mRNA, complete cds. | |
| 700353860H1 | g17603 | 11 | −2 | gb105allp | 60S RIBOSOMAL PROTEIN L26 | |
| 700383107H1 | g1145295 | 48 | −14 | gb105allp | cytochrome c oxidase subunit VIII precursor | |
| 700382165H1 | g1575129 | 87 | −76 | gb105pln | Zea mays lumenal binding protein cBiPe3 mRNA, complete cds. | |
| 700355153H1 | g168569 | 48 | −34 | gb105pln | Zea mays putative phenylalanine ammonia lyase mRNA, partial cds. | |
| 700355193H1 | g486253 | 29 | −13 | gb105pln | Yeast (Saccharomyces cerevisiae) chromosome XI reading frame ORF YKL148c. | |
| 700383058H1 | g757534 | 16 | −11 | gb105eukp | ara12; subtilisin-like protease | |
| 700382281H1 | g16836 | 10 | 3 | gb105allp | probable protease inhibitor P322 precursor (Bowman-Birk) | |
| 700354387H1 | g2459533 | 15 | −4 | gb105eukp | Ua-FatB1; acyl-ACP thioesterase | |
| 700353072H1 | g1420585 | 15 | −0 | gb105eukp | CRL13 | |
| 700382620H1 | g166972 | 20 | −4 | gb105pln | Barley acyl carrier protein I (ACP I) mRNA, complete cds. | |
| 700382344H1 | g414702 | 25 | −6 | gb105pln | H. lanatus mRNA for allergen Hol-lI. | |
| 700353616H1 | g558651 | 15 | 8 | gb105pln | T. aestivum VDAC3 mRNA for voltage dependent anion channel. | |
| 700357440H1 | g486357 | 13 | 16 | gb105pln | Yeast (Saccaromyces cerevisiae) chromosome XI reading frame ORF YKL202w. | |
| 700355611H1 | g2208961 | 24 | −0 | gb105pln | O. sativa mRNA for signal recognition particle subunit 14. | |
| 700353132H1 | g289189 | 13 | 10 | gb105pln | Arabidopsis thaliana calcium dependent protein kinase (AK1 isoform) mRNA, | |
| | | | | | complete cds. | |
| 700353231H1 | g2827001 | 49 | −33 | gb105pln | Triticum aestivum 70 kDa heat shock protein (TaHSP70d) mRNA, complete cds. | |
| 700354945H1 | g312180 | 43 | −47 | gb105pln | Z. mays GapC4 gene. | |
| 700356510H1 | g22427 | 26 | −50 | gb105pln | Z. mays gene for polygalacturonase (clone 65). | |
| 700382374H1 | g1183936 | 16 | 2 | gb105pln | P. sativum 5S rRNA gene. | |
| 700354919H1 | g1184771 | 47 | 5 | gb105pln | Zea mays glyceraldehyde-3-phosphate dehydrogenase GAPC2 (gpc2) mRNA, | |
| | | | | | complete cds. | |
| 700352687H1 | g2160318 | 59 | −50 | gb105pln | Rice mRNA for ribosomal protein L9, partial sequence. | |
| 700352738H1 | g2288886 | 33 | −19 | gb105pln | Arabidopsis thaliana mRNA for mevalonate diphosphate decarboxylase. | |
| 700383160H1 | g829147 | 66 | −69 | gb105pln | Z. mays gene for cyclophilin. | |
| 700382317H1 | g166923 | 21 | −0 | gb105pln | Arabidopsis thaliana ubiquitin carrier protein (UBC1) mRNA, complete cds. | |
| 700382305H1 | g1335202 | 4 | 8 | gb105allp | beta2/S laminin chain | |
| 700353107H1 | g2645166 | 32 | −13 | gb105pln | Oryza sativa mRNa, similar to ubiquitin conjugating enzyme. | |
| 700357168H1 | g288373 | 83 | −91 | gb105pln | Z. mays PGg6 gene for polygalacturonase. | |
| 700356759H1 | g1666233 | 26 | 10 | gb105pln | Pisum sativum actin (PEAc14) mRNA, complete cds. | |
| 700356038H1 | g1906832 | 9 | 4 | gb105eukp | C48D5.2 | |
| 700352974H1 | g2352565 | 77 | −6 | gb105eukp | Hsp82; 82 kDa heat shock protein | |
| 700353260H1 | g468524 | 24 | 1 | gb105eukp | NADPH thioredoxin reductase | |
| 700357452H1 | g218128 | 26 | −27 | gb105pln | Rice mRNA for succinyl-CoA synthetase (475 gene), partial sequence. | |
| 700353959H1 | g1041708 | 12 | 1 | gb105eukp | At-EXP2; induces extension (creep) in plant cell walls; expansin At-EXP2 | |
| 700352991H1 | g1065907 | 28 | −1 | gb105pln | Pisum sativum chloroplast processing enzyme mRNA, nuclear gene encoding chloroplast | |
| | | | | | protein, complete cds. | |
| 700356180H1 | g2384757 | 34 | −13 | gb105pln | Oryza sativa GDP dissociation inhibitor protein OsGDI1 (OsGDI1) mRNA, | |
| | | | | | complete cds. | |
| 700383050H1 | g416251 | 31 | −36 | gb105pln | Rice mRNA for acetohydroxy acid reductoisomerase, partial sequence. | |
| 700352870H1 | g473359 | 43 | −30 | gb105pln | P. pratense mRNA for Phl pI allergen. | |
| 700355948H1 | g1752864 | 14 | −11 | gb105eukp | E03H4.8 | |
| 700353276H1 | g444022 | 46 | −39 | gb105pln | N. tabacum mRNA for pyruvate kinase. | |
| 700282517H2 | g1518539 | 22 | −11 | gb105pln | Glycine max UDP-glucose dehydrogenase mRNA, complete cds. | |
| 700382952H1 | g2154729 | 11 | 5 | gb105pln | C. dactylon mRNA for profilin 1. | |
| 700352951H1 | g902583 | 53 | −9 | gb105pln | Zea mays clone MubG1 ubiquitin gene, complete cds. | |
| 700355216H1 | g2415701 | 59 | −58 | gb105pln | P. pratense mRNA for profilin 4. | |
| 700382741H1 | g32467 | 26 | −1 | gb105allp | 71 Kd heat shock cognate protein | |
| 700353054H1 | g19065 | 6 | 8 | gb105allp | nitrate reductase (NAD(P)H) | |
| 700353591H1 | g2827698 | 26 | −1 | gb105pln | Arabidopsis thaliana DNA chromosome 4, BAC clone F6H11 (ESSAII project). | |
| 700382875H1 | g288378 | 44 | −33 | gb105pln | Z. mays gene PG for polygalacturonase. | |
| 700382196H1 | g1513227 | 28 | −19 | gb105pln | Brassica napus myo-inositol 1-phosphate synthase mRNA, complete cds. | |
| 700354434H1 | g644492 | 35 | −33 | gb105pln | Corn elongation factor 1alpha gene, complete cds. | |
| 700356461H1 | g16427 | 16 | 7 | gb105eukp | protease inhibitor II | |
| 700354692H1 | g459197 | 52 | −36 | gb105pln | Gossypium hirsutum vacuolar H+-ATPase subunit B mRNA, complete cds. | |
| 700282553H2 | g2342496 | 10 | 7 | gb105eukp | bromelain; EC 3.4.4.24 | |
| 700353150H1 | g2264312 | 17 | −1 | gb105pln | Arabidopsis thaliana genomic DNA, chromosome 5, P1 clone: MOK16, complete | |
| | | | | | sequence. | |
| 700383075H1 | g974781 | 41 | −28 | gb105pln | C. blumei kinetoplast met gene for cobalamine-independent methionine synthase. | |
| 700382304H1 | g248336 | 39 | −79 | gb105pln | polyubiquitin [maize, Genomic, 3841 nt]. | |
| 700355467H1 | g557472 | 10 | 2 | gb105allp | arabinosidase | |
| 700353016H1 | g600768 | 41 | 9 | gb105pln | Oryza sativa cyclophilin 2 (Cyp2) mRNA, complete cds. | |
| 700382067H1 | g20818 | 24 | −13 | gb105pln | P. sativum mRNA for p34 kinase-related protein (cdc2 homologue). | |
| 700382649H1 | g1638835 | 7 | 7 | gb105allp | vacuolar-type H(+)-ATPase 115 kDa subunit | |
| 700356438H1 | g1173556 | 24 | 13 | gb105pln | Oryza sativa Ory s 1 mRNA, complete cds. | |
| 700382302H1 | g1532162 | 12 | 14 | gb105pln | Arabidopsis thaliana AT.I.24-1, AT.I.24-2, AT.I.24-3, AT.I.24-4, AT.I.24-5, AT.I.24-6, | |
| | | | | | AT.I.24-9 and AT.I.24-14 genes, partial cds, AT.I.24-7, ascorbate peroxidase | |
| | | | | | (ATHAPX1), EF-1-alpha-A1, -A2 and -A3 (EF-1alpha) and AT.I.24-13 genes, | |
| | | | | | complete cds. | |
| 700354821H1 | g1336097 | 13 | 7 | gb105allp | pyruvate dehydrogenase E1beta | |
| 700352725H1 | g2655157 | 21 | −27 | gb105eukp | Cyp1; cyclophilin 1 | |
| 700382707H1 | g500734 | 21 | −6 | gb105eukp | F42A10.1 | |
| 700352908H1 | g168797 | 30 | −3 | gb105pln | Neurospora crassa ribosomal protein (crp-5) gene, 5′ end. | |
| 700356459H1 | g22458 | 93 | −45 | gb105pln | Z. mays pollen specific mRNA C-terminal (clone 4H7). | |
| 700353633H1 | g2342673 | 15 | 13 | gb105pln | Sequence of BAC F7G19 from Arabidopsis thaliana chromosome 1, complete sequence. | |
| 700354642H1 | g2246625 | 11 | 0 | gb105eukp | protein kinase | |
| 700355977H1 | g1747293 | 50 | −35 | gb105pln | Rice mRNA for vacuolar H+-pyrophosphatase, complete cds. | |
| 700356520H1 | g2245126 | 20 | 16 | gb105pln | Arabidopsis thaliana DNA chromosome 4, ESSA I contig fragment No. 9. | |
| 700355842H1 | g1302311 | 7 | 6 | gb105eukp | ATX1 | |
| 700353049H1 | g2853071 | 23 | 15 | gb105pln | Arabidopsis thaliana DNA chromosome 4, BAC clone F24J7 (ESSAII project). | |
| 700282543H2 | g16931 | 12 | 6 | gb105allp | 60S ribosomal protein L32 | |
| 700354330H1 | g397958 | 33 | −48 | gb105pln | Z. mays mRNA for beta-amylase | |
| 700382048H1 | g287829 | 43 | −71 | gb105pln | Z. mays gene for polygalacturonase. | |
| 700354420H1 | g1184598 | 14 | −2 | gb105eukp | T11G6.8 | |
| 700382474H1 | g433041 | 16 | 13 | gb105pln | Zea mays W-22 clone PREM-1B retroelement PREM-1, partial sequence. | |
| 700383095H1 | g248338 | 52 | −72 | gb105pln | polyubiquitin [maize, Genomic, 3493 nt]. | |
| 700355972H1 | g623600 | 31 | −20 | gb105allp | C protein | |
| 700353218H1 | g2198410 | 6 | −0 | gb105eukp | C31C9.2 | |
| 700356759H1 | g20321 | 40 | −1 | gb105pln | Oryza sativa RAc1 mRNA for actin. | |
| 700382764H1 | g398848 | 60 | −57 | gb105pln | Z. mays mRNA for beta 5 tubulin. | |
| 700354963H1 | g22149 | 47 | −16 | gb105pln | Z. mays mRNA for alpha-tubulin 3. | |
| 700382319H1 | g22416 | 80 | −75 | gb105pln | Z. mays mRNA for polygalacturonase (clone PG1). | |
| 700353151H1 | g21477 | 25 | −3 | gb105eukp | hsp70; 70-kD heat shock protein | |
| 700355955H1 | g2245566 | 24 | 1 | gb105allp | NADH dehydrogenase III | |
| 700382035H1 | g20185 | 33 | −65 | gb105pln | O. sativa mRNA for calmodulin. | |
| 700354406H1 | g577062 | 43 | 7 | gb105allp | glutathione peroxidase | |
| 700356114H1 | g2317873 | 17 | 9 | gb105pln | Prunus armeniaca Rab7 GTP binding protein mRNA, complete cds. | |
| 700354712H1 | g1514638 | 17 | 5 | gb105pln | S. oleracea mRNA for alpha-glucan phosphorylase. | |
| 700353950H1 | g288366 | 42 | −45 | gb105pln | Z. mays PGg14 gene for polygalacturonase. | |
| 700356595H1 | g22418 | 85 | −56 | gb105pln | Z. mays gene for polygalacturonase (clone 17). | |
| 700355983H1 | g1769568 | 15 | 5 | gb105eukp | FUM1; fumarase; EC 4.2.1.2 | |
| 700356053H1 | g603220 | 32 | −21 | gb105pln | Medicago sativa 6-phosphogluconate dehydrogenase mRNA, complete cds. | |
| 700356668H1 | g2341023 | 44 | −4 | gb105pln | Sequence of BAC F19P19 from Arabidopsis thaliana chromosome 1, complete sequence. | |
| 700382816H1 | g288378 | 31 | −9 | gb105pln | Z. mays gene PG for polygalacturonase. | |
| 700355487H1 | g1184598 | 21 | −7 | gb105eukp | T11G6.8 | |
| 700382152H1 | g1572767 | 11 | 7 | gb105eukp | F29B9.5 | |
| 700356253H1 | g22312 | 59 | −0 | gb105pln | Maize ABA-inducible gene for glycine-rich protein (ABA = abscisic acid). | |
| 700383172H1 | g386857 | 59 | −11 | gb105allp | laminin receptor | |
| 700383123H1 | g2154729 | 42 | −51 | gb105pln | C. dactylon mRNA for profilin 1. | |
| 700382886H1 | g22425 | 50 | −58 | gb105pln | Z. mays gene for polygalacturonase (clone 47). | |
| 700353508H1 | g1182006 | 55 | −50 | gb105pln | H. vulgare mRNA for putative zinc finger protein. | |
| 700356445H1 | g1167835 | 22 | 6 | gb105pln | H. lanatus mRNA for Hol lI protein. | |
| 700382460H1 | g1872523 | 20 | −5 | gb105eukp | LSD1; negative regulator of cell death; zinc-finger protein Lsd1 | |
| 700356230H1 | g1546918 | 73 | −58 | gb105pln | Z. mays mRNA for translation initiation factor 5A. | |
| 700382034H1 | g601787 | 25 | −8 | gb105eukp | AFC1; activator of yeast transcription factor STE12; protein kinase | |
| 700355876H1 | g1550739 | 35 | −25 | gb105pln | A. thaliana mRNA for GDP dissociation inhibitor. | |
| 700356137H1 | g303838 | 44 | −26 | gb105pln | Rice mRNA for 40S subunit ribosomal protein, complete cds. | |
| 700353183H1 | g288378 | 27 | −42 | gb105pln | Z. mays gene PG for polygalacturonase. | |
| 700352933H1 | g22416 | 98 | −21 | gb105pln | z. mays mRNA for polygalacturonase (clone PG1). | |
| 700352802H1 | g603403 | 9 | 5 | gb105eukp | YER163C; Yer163cp | |
| 700354555H1 | g166548 | 27 | 11 | gb105pln | Avena sativa vacuolar H+-ATPase 16 kDa proteolipid subunit (vatp-P1) mRNA, | |
| | | | | | complete cds. | |
| 700355520H1 | g415896 | 4 | 7 | gb105eukp | PHL PII, pollen allergen | |
| 700354517H1 | g1314677 | 22 | 7 | gb105allp | Sec61p gamma homolog | |
| 700353562H1 | g168490 | 63 | −21 | gb105pln | Maize glutathione S-transferase (GST-I) mRNA, complete cds. | |
| 700355401H1 | g409070 | 7 | 5 | gb105allp | HBp15/L22 | |
| 700382379H1 | g288373 | 94 | −76 | gb105pln | Z. mays PGg6 gene for polygalacturonase. | |
| 700382344H1 | g1167835 | 26 | −8 | gb105pln | H. lanatus mRNA for Hol lI protein. | |
| 700382305H1 | g957312 | 4 | 8 | gb105allp | laminin beta 2 chain, S-laminin [human, placenta, Peptide, 1798 aa] | |
| 700352862H1 | g1841464 | 26 | 7 | gb105eukp | SF3; LIM-domain SF3 protein | |
| 700382279H1 | g2098713 | 11 | 8 | gb105eukp | pectinesterase | |
| 700282489H2 | g755149 | 23 | 11 | gb105pln | Gossypium hirsutum vacuolar H+-ATPase proteolipid (16 kDa) subunit (cva16-4) | |
| | | | | | mRNA, complete cds. | |
| 700282486H2 | g473168 | 22 | −8 | gb105pln | S. tuberosum E1 alpha subunit of pyruvate dehydrogenase (lipamide). | |
| 700355409H1 | g1841464 | 11 | 5 | gb105eukp | SF3; LIM-domain SF3 protein | |
| 700354710H1 | g2529657 | 17 | −9 | gb105pln | Arabidopsis thaliana chromosome II BAC T30B22 genomic sequence, coomplete | |
| | | | | | sequence. | |
| 700354551H1 | g313139 | 38 | 6 | gb105pln | Z. mays ZmPRO2 mRNA for profilin 2. | |
| 700355054H1 | g405634 | 89 | −57 | gb105pln | Z. mays zmcpt mRNA triose phosphate/phosphate translocator. | |
| 700355346H1 | g1181672 | 19 | −10 | gb105pln | Sorghum bicolor heat shock protein 70 cognate (hsc70) mRNA, partial cds. | |
| 700354963H1 | g485376 | 64 | −29 | gb105pln | Zea mays alpha-3-tubulin gene, complete cds. | |
| 700352888H1 | g1872521 | 36 | −8 | gb105eukp | PSD1; negative regulator of cell death; zinc-finger protein Lsd1 | |
| 700282545H2 | g1815758 | 14 | 15 | gb105pln | Phalaris coerulescens pollen-specific protein mRNA, complete cds. | |
| 700356190H1 | g2623962 | 20 | 5 | gb105allp | isocitrate dehydrogenase (NADP+) | |
| 700354479H1 | g1399276 | 20 | −1 | gb105pln | Arabidopsis thaliana calmodulin-domain protein kinase CDPK isoform 7 (CPK7) | |
| | | | | | mRNA, complete cds. | |
| 700356468H1 | g2245024 | 14 | 6 | gb105eukp | cyanohydrin lyase homolog | |
| 700382256H1 | g1100224 | 50 | −29 | gb105pln | Pinus sylvestris chloroplast NAD+-dependent glyceraldehyde-3-phosphate dehydrogenase | |
| | | | | | mRNA, complete cds. | |
| 700354871H1 | g886404 | 34 | −33 | gb105pln | Oryza sativa MADS-box protein (MADS3) mRNA, complete cds. | |
| 700355015H1 | g169295 | 38 | −11 | gb105pln | Pharbitis nil heat shock protein 83 (Hsp83) gene, complete cds. | |
| 700382669H1 | g439480 | 64 | −56 | gb105pln | Barley mRNA for ferrochelatase. | |
| 700382901H1 | g18260 | 21 | 4 | gb105eukp | cs DnaJ-1 | |
| 700382528H1 | g886404 | 18 | 5 | gb105pln | Oryza sativa MADS-box protein (MADS3) mRNA, complete cds. | |
| 700353257H1 | g168639 | 36 | −72 | gb105pln | Zea mays autonomous transposable element En-1 mosaic protein gene, complete cds. | |
| 700382927H1 | g2316016 | 20 | 3 | gb105allp | MRP-like ABC transporter | |
| 700282491H2 | g299191 | 36 | −15 | gb105pln | 18S ribosomal RNA [Aramanthus caudatus, Genomic, 416 nt, segment 1 of 4]. | |
| 700382845H1 | g2058277 | 21 | −13 | gb105pln | A. thaliana mRNA for AtRan1 protein. | |
| 700356094H1 | g1632767 | 13 | −3 | gb105pln | Maize mRNA for calcium dependent protein kinase, complete cds. | |
| 700353272H1 | g2618603 | 14 | 9 | gb105pln | Arabidopsis thaliana genomic DNA, chromosome 5, P1 clone: MSL3, complete | |
| | | | | | sequence. | |
| 700352957H1 | g2781432 | 65 | −56 | gb105pln | Oryza sativa subsp. joponica RSW1-like cellulose synthase catalytic subunit mRNA, | |
| | | | | | partial cds. | |
| 700355840H1 | g304668 | 24 | −4 | gb105eukp | ben | |
| 700382101H1 | g2245394 | 32 | −14 | gb105eukp | putative transcription factor; ARF1-binding protein | |
| 700382331H1 | g555699 | 31 | −28 | gb105pln | Arabidopsis thaliana phosphatidylinositol 3-kinase (AtVps34) mRNA, complete cds. | |
| 700353072H1 | g1931623 | 15 | −0 | gb105eukp | PCS1; proteasome cap subunit | |
| 700353568H1 | g533775 | 30 | 4 | gb105eukp | Mha1; H(+)-transporting ATPase | |
| 700354727H1 | g1762944 | 28 | −16 | gb105pln | Nicotiana tabacum ORF mRNA, complete cds. | |
| 700382436H1 | g2370518 | 7 | 6 | gb105allp | ribosomal protein | |
| 700354886H1 | g16836 | 12 | 6 | gb105allp | probably protease inhibitor P322 precursor (Bowman-Birk) | |
| 700355938H1 | g2369766 | 12 | 8 | gb105eukp | hypothetical protein | |
| 700352954H1 | g416265 | 48 | −15 | gb105pln | Rice mRNA for ribosomal protein A2, partial sequence. | |
| 700353202H1 | g596079 | 87 | −83 | gb105pln | Zea mays thiamine biosynthetic enzyme (thi1-2) mRNA, complete cds. | |
| 700355865H1 | g998429 | 28 | −49 | gb105pln | GRF1 = general regulatory factor [Zea mays, XL80, Genomic, 5348 nt]. | |
| 700357423H1 | g288366 | 55 | −64 | gb105pln | Z. mays PGg14 gene for polygalacturonase. | |
| 700352868H1 | g551301 | 16 | 5 | gb105eukp | SSE1; Sse1 protein | |
| 700352788H1 | g561923 | 16 | −1 | gb105eukp | M01F1.2 | |
| 700354435H1 | g2275008 | 36 | −28 | gb105pln | Hordeum vulgare mRNA for expressed sequence tag. | |
| 700382240H1 | g2326873 | 26 | −23 | gb105pln | S. tuberosum mRNA for plastidic ATP/ADP-transporter. | |
| 700355555H1 | g644491 | 47 | −63 | gb105pln | Corn mRNA for elongation factor 1A. | |
| 700355983H1 | g1488652 | 16 | 3 | gb105eukp | fum1; fumarase; EC 4.2.1.2 | |
| 700352829H1 | g2578365 | 19 | 2 | gb105allp | GTP cyclohydrolase II/3,4-dihydroxy-2-butanone-4-phosphate synthase | |
| 700356585H1 | g217910 | 42 | −32 | gb105pln | Carrot mRNA for nuclear antigen 21D7. | |
| 700353237H1 | g22747 | 28 | −6 | gb105pln | L. longiflorum mRNA for actin depolymerizing factor. | |
| 700355823H1 | g2623989 | 36 | −22 | gb105pln | Arabidopsis thaliana mRNA for ferrochelatase. | |
| 700352613H1 | g177207 | 17 | 8 | gb105allp | 4F2 antigen heavy chain | |
| 700354469H1 | g19255 | 37 | −23 | gb105pln | Lycopersicon esculentum hsc-1 mRNA for heat shock protein 70 kD. | |
| 700352883H1 | g1895083 | 59 | −24 | gb105pln | Zea mays golgi associated protein se-wap41 mRNA, complete cds. | |
| 700357450H1 | g1184775 | 31 | −30 | gb105pln | Zea mays glyceraldehyde-3-phosphate dehydrogenase (gpc4) mRNA, complete cds. | |
| 700353263H1 | g20255 | 55 | −54 | gb105pln | O. sativa gene for heat shock protein 82 HSP82. | |
| 700356773H1 | g881521 | 37 | −1 | gb105eukp | AtHXK1; hexokinase 1; EC 2.7.1.1 | |
| 700352826H1 | g288612 | 15 | −3 | gb105eukp | PG; polygalacturonase | |
| 700356084H1 | g288373 | 50 | −58 | gb105pln | Z. mays PGg6 gene for polygalacturonase. | |
| 700353278H1 | g1246115 | 23 | −5 | gb105pln | major allergen Pha a1 [Phalaris aquatica = canary grass, pollen, mRNA Partial, | |
| | | | | | 1088 nt]. | |
| 700353560H1 | g1002369 | 14 | −2 | gb105allp | coatomer protein | |
| 700356246H1 | g1143864 | 43 | −10 | gb105eukp | catalyzes the release of either giberellin or cyanogenic substances from their gluco- | |
| | | | | | conjugates; beta glucosidase; EC 3.2.1.21 | |
| 700354824H1 | g1388084 | 22 | −3 | gb105eukp | TRX5; thioredoxin h | |
| 700282482H2 | g1799575 | 8 | 6 | gb105allp | DNA GYRASE SUBUNIT A (EC 5.99.1.3). | |
| 700382055H1 | g790582 | 18 | 4 | gb105pln | Arabidospsis thaliana putative pathogenesis-related protein (ATOZI1) mRNA, | |
| | | | | | complete cds. | |
| 700353987H1 | g22425 | 53 | 6 | gb105pln | Z. mays gene for polygalacturonase (clone 47). | |
| 700356510H1 | g22456 | 26 | −46 | gb105pln | Z. mays pollen specific mRNA C-terminal (clone 3C12). | |
| 700357431H1 | g19103 | 47 | −37 | gb105pln | H. vulgare mRNA for ribosomal protein L17-2. | |
| 700355915H1 | g288378 | 100 | −87 | gb105pln | Z. mays gene PG for polygalacturonase. | |
| 700356258H1 | g476002 | 74 | −71 | gb105pln | Hordeum vulgare HSP70 mRNA, complete cds. | |
| 700354263H1 | g439480 | 57 | −51 | gb105pln | Barley mRNA for ferrochelatase. | |
| 700355509H1 | g2282583 | 52 | −81 | gb105pln | Zea mays elongation factor 1-alpha (EF1-A) mRNA, complete cds. | |
| 700356502H1 | g1747418 | 75 | −50 | gb105pln | Z. mays mRNA for eukaryotic initiation factor-5. | |
| 700382235H1 | g313137 | 70 | −89 | gb105pln | Z. mays ZmPRO1 mRNA for profilin 1. | |
| 700353918H1 | g288611 | 43 | −43 | gb105pln | Z. mays PG gene for polygalacturonase. | |
| 700353688H1 | g1815759 | 16 | −0 | gb105eukp | pollen-specific protein | |
| 700383130H1 | g22356 | 11 | −26 | gb105pln | Maize mRNA for light-harvesting chlorophyll a/b binding protein LHCP. | |
| 700383159H1 | g22426 | 8 | 6 | gb105allp | polygalacturonase | |
| 700382242H1 | g1370195 | 15 | 11 | gb105pln | L. japonicus mRNA for small GTP-binding protein, RAB8D. | |
| 700354682H1 | g22458 | 14 | −20 | gb105pln | Z. mays pollen specific mRNA C-terminal (clone 4H7). | |
| 700383004H1 | g143814 | 9 | 3 | gb105allp | HisH | |
| 700352603H1 | g248338 | 98 | −86 | gb105pln | polyubiquitin [maize, Genomic 3439 nt]. | |
| 700353206H1 | g16211 | 22 | −4 | gb105eukp | CNX1; calnexin homolog | |
| 700352790H1 | g600710 | 16 | 7 | gb105eukp | SAR1 | |
| 700382279H1 | g2098709 | 11 | 8 | gb105allp | pectinesterase | |
| 700357413H1 | g499111 | 39 | −29 | gb105pln | Flaveria bidentis clone pNDK-b nucleoside diphosphate kinase mRNA, complete cds. | |
| 700355461H1 | g644492 | 80 | −67 | gb105pln | Corn elongation factor 1alpha gene, complete cds. | |
| 700355501H1 | g1498391 | 33 | −26 | gb105pln | Zea mays actin (Maz65) gene, partial cds. | |
| 700383128H1 | g22423 | 29 | −22 | gb105pln | Z. mays mRNA for polygalacturonase (clone PG4). | |
| 700355748H1 | g1620661 | 39 | −39 | gb105pln | Triticum aestivum 1,4-alpha-D-glucan 6-alpha-D-(1,4-alpha-D-glucanotransferase | |
| | | | | | mRNA, complete cds. | |
| 700282447H2 | g927571 | 47 | −37 | gb105pln | Z. mays mRNA for calreticulin precursor. | |
| 700382327H1 | g218129 | 25 | 0 | gb105pln | Rice mRNA for F1-ATPase (480 gene), partial sequence. | |
| 700352788H1 | g963095 | 16 | −1 | gb105eukp | 60S ribosomal protein L13A | |
| 700382892H1 | g312180 | 24 | −58 | gb105pln | Z. mays GapC4 gene. | |
| 700352826H1 | g22428 | 14 | −4 | gb105eukp | polygalacturonase | |
| 700355188H1 | g968901 | 69 | −63 | gb105pln | Rice mRNA for ribosomal protein S8, complete cds. | |
| 700353960H1 | g1053058 | 36 | −36 | gb105pln | Triticum aestivum histone H3 gene, partial cds, clone W2. | |
| 700354931H1 | g22427 | 93 | −60 | gb105pln | Z. mays gene for polygalacturonase (clone 65). | |
| 700355407H1 | g2335104 | 23 | −5 | gb105eukp | T11A07.16; villin isolog | |
| 700355095H1 | g20163 | 54 | −27 | gb105pln | O. sativa Rr15 mRNA for 5S ribosomal RNA. | |
| 700354602H1 | g550060 | 22 | −6 | gb105allp | GTP-binding protein | |
| 700355847H1 | g1173829 | 36 | −29 | gb105pln | Arabidopsis thaliana meristem L1 layer homeobox protein (ATML1) mRNA, | |
| | | | | | complete cds. | |
| 700356169H1 | g22427 | 57 | −61 | gb105pln | Z. mays gene for polygalacturonase (clone 65). | |
| 700355236H1 | g18296 | 59 | −1 | gb105pln | Water melon mMDH mRNA for mitochondrial malate dehydrogenase (EC 1.1.1.37). | |
| 700354856H1 | g21599 | 19 | −6 | gb105eukp | UTP-glucose-1-phosphate uridylyltransferase; EC 2.7.7.9 | |
| 700355015H1 | g300082 | 37 | −6 | gb105pln | hsp82 = 82 kda heat shock protein [Zea mays, seedling, leaves, Genomic, 3468 nt]. | |
| 700354603H1 | g22458 | 21 | 2 | gb105pln | Z. mays pollen specific mRNA C-terminal (clone 4H7). | |
| 700353260H1 | g757510 | 18 | 0 | gb105eukp | cys-9; Thioredoxin Reductase (NADPH); EC 1.6.4.5 | |
| 700356177H1 | g2828188 | 20 | 5 | gb105pln | Arabidopsis thaliana genomic DNA, chromosome 5, TAC clone: K3I3, complete | |
| | | | | | sequence. | |
| 700352973H1 | g1246116 | 24 | 1 | gb105allp | major allergen Pha a1 [Phalaris aquatica = canary grass, pollen, Peptide, 269 aa] | |
| 700356190H1 | g1750380 | 18 | 5 | gb105eukp | EgICDH; NADP-isocitrate dehydrogenase | |
| 700355088H1 | g415896 | 9 | 7 | gb105eukp | PHL PII, pollen allergen | |
| 700353562H1 | g168486 | 76 | −32 | gb105pln | Maize glutathione S-transferase gene (GST-I), exon 1. | |
| 700352922H1 | g1679658 | 16 | −10 | gb105eukp | gamma glutamyl hydrolase | |
| 700355046H1 | g22149 | 82 | −76 | gb105pln | Z. mays mRNA for alpha-tubulin 3. | |
| 700383193H1 | g255568 | 60 | −49 | gb105pln | pollen specific protein [Zea mays = corn, mRNA, 943 nt]. | |
| 700355740H1 | g414702 | 25 | −9 | gb105pln | H. lanatus mRNA for allergen Hol-lI. | |
| 700383138H1 | g1002914 | 31 | −25 | gb105pln | Oryza sativa cap-binding protein p26 mRNA, complete cds. | |
| 700382924H1 | g1418319 | 12 | 1 | gb105allp | CER1-like | |
| 700355338H1 | g1930071 | 35 | −22 | gb105pln | Oryza sativa thioredoxin h mRNA, complete cds. | |
| 700356893H1 | g974781 | 26 | −2 | gb105pln | c. blumei kinetoplast met gene for cabalamine-independent methionine synthase. | |
| 700353930H1 | g405634 | 76 | −17 | gb105pln | z. mays zmcpt mRNA triose phosphate/phosphate translocator. | |
| 700355966H1 | g516306 | 21 | 5 | gb105eukp | Su(var)3-9; 51 KDa protein | |
| 700353685H1 | g886400 | 23 | 1 | gb105pln | Oryza sativa MADS-box protein (MADS2) mRNA, complete cds. | |
| 700353543H1 | g575730 | 94 | −70 | gb105pln | Z. mays mRNA for transmembrane protein. | |
| 700352783H1 | g996081 | 13 | 4 | gb105eukp | syx1A; syntaxin 1A | |
| 700356507H1 | g1184773 | 39 | −13 | gb105pln | Zea mays glyceraldehyde-3-phosphate dehydrogenase GAPC3 (gpc3) mRNA, | |
| | | | | | complete cds. | |
| 700382015H1 | g16511 | 24 | −40 | gb105pln | A. thaliana mRNA for suppressor-like protein. | |
| 700282520H2 | g1518539 | 42 | −20 | gb105pln | Glycine max UDP-glucose dehydrogenase mRNA, complete cds. | |
| 700356964H1 | g2654011 | 42 | 1 | gb105allp | Rho-like GTP binding protein | |
| 700354891H1 | g2827513 | 17 | 10 | gb105pln | Arabidopsis thaliana DNA chromosome 4, BAC clone F8F16 (ESSAII project). | |
| 700355312H1 | g2315152 | 28 | −24 | gb105pln | Arabidopsis thaliana mRNA for MAP3K gamma protein kinase. | |
| 700382357H1 | g435428 | 30 | −8 | gb105pln | Arabidopsis thaliana GTP-binding protein mRNA. | |
| 700354944H1 | g312180 | 46 | −49 | gb105pln | Z. mays GapC4 gene. | |
| 700355976H1 | g218152 | 19 | −2 | gb105pln | Rice serine cbp3 gene for carboxypeptidase III. | |
| 700356086H1 | g1857256 | 9 | 3 | gb105eukp | mudrAzc; MURAZC | |
| 700382901H1 | g1125690 | 24 | 1 | gb105pln | S. tuberosum mRNA for DnaJ protein. | |
| 700352911H1 | g1277236 | 10 | 7 | gb105eukp | SEC13; Sec13p | |
| 700354267H1 | g393449 | 29 | −4 | gb105pln | S. cereale mRNA for beta-amylase. | |
| 700353247H1 | g2244881 | 9 | 7 | gb105eukp | PDR5-like ABC transporter | |
| 700356033H1 | g453244 | 10 | 4 | gb105allp | catechol O-methyltransferase | |
| 700382373H1 | g1155241 | 11 | 17 | gb105pln | Dianthus caryophyllus S-adenosylmethionine 2 decarboxylase mRNA, complete cds. | |
| 700282442H2 | g1314048 | 20 | 6 | gb105pln | O. sativa mRNA for archain/delta-COP. | |
| 700382933H1 | g1651457 | 5 | 7 | gb105allp | Aminopeptidase N | |
| 700355840H1 | g546724 | 24 | −4 | gb105eukp | ben | |
| 700356174H1 | g169988 | 28 | −5 | gb105pln | Glycine max NADP-specific isocitrate dehydrogenase (idh1) mRNA, 3′ end. | |
| 700353560H1 | g1237029 | 14 | −2 | gb105allp | alpha-cop protein | |
| 700352660H1 | g414702 | 26 | −10 | gb105pln | H. lanatus mRNA for allergen Hol-lI. | |
| 700354265H1 | g1354861 | 24 | −4 | gb105eukp | COI; cytochrome oxidase | |
| 700353690H1 | g602425 | 38 | −22 | gb105pln | Mesembryanthemum crystallinum phosphoglyceromutase (pgm) gene, complete cds. | |
| 700353528H1 | g22418 | 90 | −84 | gb105pln | Z. mays gene for polygalacturonase (clone 17). | |
| 700354366H1 | g498039 | 19 | 0 | gb105pln | Senecio odorus mRNA, complete ORF. | |
| 700356089H1 | g2264311 | 15 | −14 | gb105pln | Arabidopsis thaliana genomic DNA, chromosome 5, P1 clone: MLN1, complete | |
| | | | | | sequence. | |
| 700383167H1 | g1486286 | 19 | 6 | gb105pln | C. sativus mRNA for T-complex polypeptide 1. | |
| 700354368H1 | g2130984 | 28 | −19 | gb105pln | B. napus mRNA for ribosomal protein S15a (pA8130). | |
| 700282471H2 | g17919 | 13 | 8 | gb105eukp | S-receptor kinase related protein | |
| 700352904H1 | g4401 | 27 | −3 | gb105pln | Yeast gene for ribosomal protein S10-2. | |
| 700382907H1 | g17561 | 18 | 3 | gb105allp | PROTEINASE INHIBITOR | |
| 700354523H1 | g972255 | 20 | 7 | gb105allp | ribosomal protein L21 | |
| 700382611H1 | g414702 | 26 | −6 | gb105pln | H. lanatus mRNA for allergen Hol-lI. | |
| 700353994H1 | g170772 | 69 | −25 | gb105pln | Triticum aestivum S-adenosyl-L-homocysteine hydrolase (SH6.2) mRNA, complete cds. | |
| 700383173H1 | g1136786 | 23 | −20 | gb105pln | Fission Yeast mRNA for elongation factor 1 alpha-C, complete cds. | |
| 700382532H1 | g2618603 | 12 | 15 | gb105pln | Arabidopsis thaliana genomic DNA, chromosome 5, P1 clone: MSL3, complete | |
| | | | | | sequence. | |
| 700356094H1 | g639721 | 13 | −4 | gb105pln | Zea mays calcium-dependent protein kinase (CDPK) gene, exons 1-7 and partial cds. | |
| 700355415H1 | g16446 | 7 | 14 | gb105pln | A. thaliana gene for photosystem II 10 kDa polypeptide. | |
| 700356114H1 | g623587 | 14 | 12 | gb105pln | Nicotiana tabacum SR1 Nt-rab7a mRNA, complete cds. | |
| 700353003H1 | g2781347 | 24 | 7 | gb105eukp | F24O1.3 | |
| 700356033H1 | g2565273 | 8 | 7 | gb105allp | o-methytransferase | |
| 700356611H1 | g1418408 | 50 | −10 | gb105pln | B. vulgaris mRNA for subunit A of the V-type ATPase. | |
| 700356115H1 | g22416 | 98 | −27 | gb105pln | Z. mays mRNA for polygalacturonase (clone PG1). | |
| 700354794H1 | g1438881 | 15 | 1 | gb105eukp | GmCK2p; EC 2.7.1.32 | |
| 700354414H1 | g433044 | 19 | 1 | gb105pln | Zea mays W-22 clone PREM-1D retroelement PREM-1, partial sequence. | |
| 700353213H1 | g288373 | 79 | −67 | gb105pln | Z. mays PGg6 gene for polygalacturonase. | |
| 700356285H1 | g3972 | 21 | −7 | gb105eukp | mitochondrial release factor 1 | |
| 700353024H1 | g2414643 | 25 | −2 | gb105pln | Yeast (Saccaromyces pombe) chromosome I cosmid c3H5. | |
| 700353268H1 | g255568 | 32 | −71 | gb105pln | pollen specific protein [Zea mays = corn, mRNA, 943 nt]. | |
| 700356438H1 | g1173557 | 30 | 7 | gb105allp | Ory s 1 | |
| 700382649H1 | g2330695 | 7 | 5 | gb105eukp | SPAC16E8.07c; vacuolar atpase | |
| 700382325H1 | g2429285 | 10 | 16 | gb105pln | Oryza sativa peroxidase (POX22.3) mRNA, complete cds. | |
| 700357409H1 | g498774 | 38 | −27 | gb105pln | Z. mays (cv DH5xDH7) hsp70-5 mRNA for heat shock protein 70. | |
| 700383037H1 | g1532072 | 19 | 5 | gb105pln | Z. mays mRNA for S-adenosylmethionine decarboxylase. | |
| 700383130H1 | g218171 | 14 | −29 | gb105pln | Rice mRNA for type I light-harvesting chlorophyll a/b binding protein of photosystem II | |
| | | | | | (LHCPII). | |
| 700353695H1 | g1911582 | 10 | 0 | gb105allp | Cyn d 1 = major allergen [Cynodon dactylon = Bermuda grass, pollen, Peptide Partial, | |
| | | | | | 246 aa] | |
| 700356576H1 | g1370324 | 38 | 3 | gb105allp | ORF YPL152w | |
| 700383062H1 | g395316 | 4 | 8 | gb105eukp | P405CR10; cytochrome P450 | |
| 700354759H1 | g17561 | 18 | 5 | gb105allp | PROTEINASE INHIBITOR | |
| 700354570H1 | g19500 | 32 | 1 | gb105pln | Lupinus polyphyllus lupin-specific pPLZ12 mRNA. | |
| 700355840H1 | g1480355 | 20 | −2 | gb105eukp | UBC13; ubiquitin-conjugating enzyme | |
| 700353823H1 | g2384759 | 22 | −13 | gb105pln | Oryza sativa GDP dissociation inhibitor protein OsGDI2 (OsGDI2) mRNa, complete cds. | |
| 700356435H1 | g20237 | 15 | 1 | gb105pln | O. sativa (rice) constitutive GOS2 gene. | |
| 700354748H1 | g2342737 | 13 | 7 | gb105eukp | T14G11.30; Ser/Thr protein kinase isolog, 5′ partial | |
| 700382823H1 | g312180 | 30 | −33 | gb105pln | Z. mays GapC4 gene. | |
| 700353084H1 | g2130943 | 23 | −2 | gb105pln | Arabidopsis thaliana mRNA for sulfate transporter, complete cds. | |
| 700352920H1 | g2264308 | 38 | 7 | gb105pln | Arabidopsis thaliana genomic DNA, chromosome 5, P1 clone: MIK22, complete | |
| | | | | | sequence. | |
| 700356479H1 | g886402 | 46 | −15 | gb105pln | Oryza sativa MADS-box protein (MADS4) mRNA, complete cds. | |
| 700353918H1 | g288378 | 35 | −28 | gb105pln | Z. mays gene PG for polygalacturonase. | |
| 700353060H1 | g1335965 | 54 | −46 | gb105pln | Zea mays acetyl CoA carboxylase mRNA, partial cds. | |
| 700354929H1 | g1167835 | 24 | −6 | gb105pln | H. lanatus mRNA for Hol lI protein. | |
| 700352637H1 | g313759 | 50 | −49 | gb105pln | Z. mays hsp 70-1 gene for heat shock protein 70. | |
| 700355733H1 | g1395190 | 23 | −1 | gb105pln | Spinacia oleracea L. mRNA for 26S proteasome ATPase subunit, complete cds. | |
| 700354457H1 | g288366 | 37 | −53 | gb105pln | Z. mays PGg14 gene for polygalacturonase. | |
| 700382947H1 | g224256 | 96 | −44 | gb105pln | Z. mays gene for polygalacturonase (clone 47). | |
| 700355971H1 | g170919 | 13 | 5 | gb105pln | Yeast (Candida maltosa) ribosomal protein L41 (LEL41) gene, complete cds. | |
| 700382331H1 | g736336 | 33 | −30 | gb105pln | Glycine max phosphatidylinositol 3-kinase mRNA, complete cds. | |
| 700355760H1 | g20168 | 50 | −32 | gb105pln | O. sativa mRNA for alcohol dehydrogenase 2. | |
| 700382567H1 | g2342683 | 44 | −22 | gb105eukp | F7G19.12 | |
| 700382122H1 | g558649 | 26 | −5 | gb105pln | T. aestivum VDAC2 mRNA for voltage dependent anion channel. | |
| 700355242H1 | g1906827 | 10 | 14 | gb105pln | A. thaliana hsp81.4 gene. | |
| 700357449H1 | g1313907 | 65 | −5 | gb105eukp | CDPK-related protein kinase | |
| 700355978H1 | g1718238 | 12 | −0 | gb105eukp | (1,4)-beta-xylan endohydrolase, isoenzyme X-II; EC 3.2.1.8 | |
| 700355519H1 | g20323 | 29 | −30 | gb105pln | O. sativa RAc1 gene for actin. | |
| 700356032H1 | g456567 | 30 | −11 | gb105pln | Pisum sativum ubiquitin conjugating enzyme (UBC4), complete cds. | |
| 700356685H1 | g22418 | 93 | −38 | gb105pln | Z. mays gene for polygalacturonase (clone 17). | |
| 700383014H1 | g414702 | 23 | −6 | gb105pln | H. lanatus mRNA for allergen Hol-lI. | |
| 700382615H1 | g1070357 | 28 | −13 | gb105pln | H. vulgare mRNA for oxalate oxidase-like protein. | |
| 700354249H1 | g407411 | 72 | −7 | gb105pln | C. roseus SAHH gene for S-adenosyl-L-homocysteine hydrolase. | |
| 700352617H1 | g313137 | 49 | −47 | gb105pln | Z. mays ZmPRO1 mRNA for profilin 1. | |
| 700356050H1 | g2258073 | 27 | −4 | gb105pln | Hordeum vulgare var. distichum soluble inorganic pyrophosphatase (Ipp) mRNA, | |
| | | | | | complete cds. | |
| 700382254H1 | g556685 | 53 | −46 | gb105pln | Z. mays mRNA for ADP-ribosylation factor. | |
| 700382242H1 | g2808637 | 15 | 8 | gb105pln | Daucus carota mRNA for Rab8-like small GTP-binding protein. | |
| 700355982H1 | g1107484 | 26 | −11 | gb105pln | A. thaliana mRNA for 40S ribosomal protein S15. | |
| 700354301H1 | g2370231 | 27 | −39 | gb105pln | Hordeum vulgare mRNA for putative acyl-CoA oxidase. | |
| 700354362H1 | g288366 | 38 | −58 | gb105pln | Z. mays PGg14 gene for polygalacturonase. | |
| 700352908H1 | g3075 | 36 | −12 | gb105pln | N. crassa mRNA for a ribosomal protein. | |
| 700355771H1 | g20635 | 19 | −3 | gb105eukp | actin | |
| 700353084H1 | g2114103 | 32 | −12 | gb105pln | Arabidopsis thaliana DNA for sulfate transporter, complete cds. | |
| 700357410H1 | g2213581 | 14 | −5 | gb105eukp | T7N9.1 | |
| 700356473H1 | g1002369 | 18 | 5 | gb105allp | coatomer protein | |
| 700382422H1 | g7758 | 29 | −12 | gb105eukp | crn | |
| 700382525H1 | g475046 | 12 | 7 | gb105eukp | putative cell wall protein | |
| 700354866H1 | g22416 | 31 | −33 | gb105pln | Z. mays mRNA for polygalacturonase (clone PG1). | |
| 700355839H1 | g18259 | 18 | −16 | gb105pln | C. sativus mRNA for cs DnaJ-1. | |
| 700382905H1 | g2746086 | 12 | −6 | gb105eukp | HvHAK1; putative high-affinity potassium transporter | |
| 700355576H1 | g927571 | 65 | −69 | gb105pln | Z. mays mRNA for calreticulin precursor. | |
| 700382640H1 | g2760173 | 20 | −4 | gb105pln | Arabidopsis thaliana genomic DNA, chromosome 5, P1 clone: MYH19, complete | |
| | | | | | sequence. | |
| 700356415H1 | g406661 | 15 | 1 | gb105allp | Calcium-binding protein | |
| 700354274H1 | g2285790 | 36 | −17 | gb105allp | p47 | |
| 700354392H1 | g22427 | 80 | −77 | gb105pln | Z. mays gene for polygalacturonase (clone 65). | |
| 700354277H1 | g181041 | 63 | −32 | gb105allp | cAMP response element regulatory protein | |
| 700382932H1 | g6711 | 25 | 1 | gb105eukp | F02A9.5 | |
| 700383151H1 | g2062155 | 29 | −11 | gb105eukp | T02O04.2; mitochondrial processing peptidase alpha subunit precursor isolog | |
| 700353231H1 | g22340 | 71 | −77 | gb105pln | Maize gene for heat shock protein 70 exon 1 (hsp70; clone pMON 9502). | |
| 700353092H1 | g1542940 | 33 | −22 | gb105pln | R. sativus L. (Saxa knacker) AACT mRNA. | |
| 700356177H1 | g1518539 | 26 | −3 | gb105pln | Glycine max UDP-glucose dehydrogenase mRNA, complete cds. | |
| 700355005H1 | g2827001 | 48 | −34 | gb105pln | Triticum aestivum 70 kDa heat shock protein (TaHSP70d) mRNA, complete cds. | |
| 700354625H1 | g1167836 | 9 | 7 | gb105eukp | hol lI; major group I allergen; protein with incomplete signal sequence | |
| 700382329H1 | g288373 | 99 | −84 | gb105pln | Z. mays PGg6 gene for polygalacturonase. | |
| 700282568H2 | g2444419 | 29 | −16 | gb105pln | Glycine max ribosome-associated protein p40 mRNA, complete cds. | |
| 700354871H1 | g854645 | 63 | −71 | gb105pln | Z. mays ZMM2 mRNA (partial). | |
| 700355160H1 | g2160155 | 16 | 15 | gb105pln | Sequence of BAC F21M12 from Arabidopsis thaliana chromosome 1, complete | |
| | | | | | sequence. | |
| 700352784H1 | g1523915 | 36 | −17 | gb105eukp | F25H2.10 | |
| 700354544H1 | g2760086 | 34 | −0 | gb105eukp | G8B7T7; leucine-rich repeat protein | |
| 700382850H1 | g664900 | 27 | −22 | gb105pln | C. plantagineum tkt3 gene for transketolase. | |
| 700354906H1 | g16427 | 25 | −3 | gb105eukp | protease inhibitor II | |
| 700356917H1 | g2677829 | 60 | −17 | gb105pln | Prunus armeniaca ribosomal protein L12 mRNA, complete cds. | |
| 700353067H1 | g1895083 | 63 | −57 | gb105pln | Zea mays golgi associated protein se-wap41 mRNA, complete cds. | |
| 700355855H1 | g21361 | 13 | 15 | gb105pln | Silene latifolia ssp. alba mRNA for ferredoxin precursor. | |
| 700382216H1 | g1805654 | 11 | 6 | gb105eukp | calmodulin-stimulated calcium-ATPase | |
| 700357106H1 | g493368 | 75 | 4 | gb105allp | S-adenosylmethionine synthetase 2 | |
| 700353222H1 | g527680 | 33 | −12 | gb105eukp | ribosomal protein S3 | |
| 700354990H1 | g16064 | 6 | 8 | gb105eukp | tam3; Tam3-transposase | |
| 700282420H2 | g1245689 | 9 | 3 | gb105eukp | F36D4.5 | |
| 700356123H1 | g414702 | 29 | −11 | gb105pln | H. lanatus mRNA for allergen Hol-lI. | |
| 700356820H1 | g1498389 | 76 | −82 | gb105pln | Zea mays actin (Maz81) gene, partial cds. | |
| 700354338H1 | g169975 | 11 | 7 | gb105eukp | vspA; vegetative storage protein | |
| 700356164H1 | g287829 | 54 | −59 | gb105pln | Z. mays gene for polygalacturonase. | |
| 700354295H1 | g3618 | 17 | −8 | gb105eukp | cyclophilin (AA 1-162) | |
| 700352862H1 | g2088643 | 28 | 6 | gb105allp | transcription factor SF3 isolog | |
| 700356113H1 | g168314 | 17 | −3 | gb105eukp | Lol pI; pollen allergen | |
| 700382079H1 | g2264304 | 30 | −30 | gb105pln | Arabidopsis thaliana genomic DNA, chromosome 5, P1 clone: MBG8, complete | |
| | | | | | sequence. | |
| 700382265H1 | g1051257 | 62 | −37 | gb105pln | Hordeum vulgare vacuolar ATPase catalytic subunit mRNA, partial cds. | |
| 700352954H1 | g790507 | 27 | 2 | gb105pln | Z. mays mRNA for 60S acidic ribosomal protein. | |
| 700355409H1 | g2088643 | 12 | 5 | gb105allp | transcription factor SF3 isolog | |
| 700282585H2 | g1167835 | 22 | −5 | gb105pln | H. lanatus mRNA for Hol lI protein. | |
| 700354921H1 | g2586083 | 11 | 8 | gb105allp | receptor kinase-like protein | |
| 700382546H1 | g398844 | 46 | −23 | gb105pln | Z. mays mRNA for beta 3 tubulin. | |
| 700356416H1 | g553123 | 12 | 2 | gb105eukp | ORF | |
| 700355006H1 | g499011 | 26 | −16 | gb105pln | S. vulgare SoAc1 mRNA. | |
| 700355484H1 | g173231 | 15 | 10 | gb105pln | Yeast (Saccharomyces cerevisiae) ribosomal 5S RNA-binding protein (YL3) gene, 5′ cds. | |
| 700354441H1 | g386812 | 30 | −19 | gb105allp | immunoglobulin kappa chain (V-J-C) | |
| 700382290H1 | g495227 | 31 | −0 | gb105eukp | TE13; acyl-ACP thioesterase | |
| 700354483H1 | g1890351 | 20 | −0 | gb105pln | A. thaliana mRNA for transcription factor L2. | |
| 700355083H1 | g902789 | 23 | −10 | gb105eukp | ILL1; ILL1 | |
| 700354492H1 | g486050 | 27 | −8 | gb105eukp | ORF YKL040c | |
| 700352978H1 | g2827001 | 65 | −6 | gb105pln | Triticum aestivum 70 kDa heat shock protein (TaHSP70d) mRNA, complete cds. | |
| 700382342H1 | g397958 | 42 | −69 | gb105pln | Z. mays mRNA for beta-amylase. | |
| 700354778H1 | g1438883 | 11 | 1 | gb105eukp | GmCK3p; EC 2.7.1.32 | |
| 700354471H1 | g2353354 | 12 | −11 | gb105eukp | cytochrome oxidase I | |
| 700356485H1 | g899607 | 92 | −56 | gb105pln | Zea mays polyubiquitin (MubC5) mRNA, complete cds. | |
| 700355865H1 | g168602 | 34 | −39 | gb105pln | Zea mays regulatory protein GF14-12 mRNA, complete cds. | |
| 700382091H1 | g22426 | 17 | −8 | gb105eukp | polygalacturonase | |
| 700353793H1 | g167040 | 22 | −16 | gb105pln | Barley elongation factor 1 alpha, (EF-1A) mRNA sequence. | |
| 700382319H1 | g288373 | 80 | −75 | gb105pln | Z. mays PGg6 gene for polygalacturonase. | |
| 700352636H1 | g1945365 | 9 | 4 | gb105allp | copper transport protein HAH1 | |
| 700354816H1 | g2244792 | 7 | 7 | gb105allp | ankyrin homolog | |
| 700354422H1 | g1167835 | 22 | −29 | gb105pln | H. lanatus mRNA for Hol lI protein. | |
| 700356170H1 | g1167835 | 22 | −2 | gb105pln | H. lanatus mRNA for Hol lI protein. | |
| 700354805H1 | g22651 | 24 | −31 | gb105pln | H. vulgare mRNA for GRP94 homologue. | |
| 700354918H1 | g22426 | 12 | 7 | gb105allp | polygalacturonase | |
| 700355245H1 | g393449 | 50 | −42 | gb105pln | S. cereale mRNA for beta-amylase. | |
| 700354260H1 | g304037 | 10 | 1 | gb105eukp | ENOD8; early nodulin | |
| 700353885H1 | g2239181 | 10 | 3 | gb105eukp | SPAC15A10.04c; hypothetical protein | |
| 700353917H1 | g22420 | 64 | 1 | gb105pln | Z. mays mRNA for polygalacturonase (clone PG2). | |
| 700354566H1 | g623585 | 13 | 3 | gb105pln | Nicotiana tabacum SR1 Nt-rab6 mRNA, complete cds. | |
| 700355163H1 | g22416 | 94 | −44 | gb105pln | Z. mays mRNA for polygalacturonase (clone PG1). | |
| 700355740H1 | g414907 | 27 | −11 | gb105pln | P. pratense mRNA for pollen allergen Ph1pI. | |
| 700355411H1 | g1771781 | 25 | −31 | gb105pln | T. aestivum mRNA for beta-amylase. | |
| 700355446H1 | g17789 | 26 | 2 | gb105allp | protein homologous to ascorbate oxidase | |
| 700356216H1 | g2191160 | 19 | 5 | gb105allp | Similar to pectinesterase | |
| 700353837H1 | g441486 | 6 | 8 | gb105allp | coatomer | |
| 700352726H1 | g514319 | 32 | −15 | gb105pln | Arabidopsis thaliana RNA polymerase II third largest subunit gene, complete cds. | |
| 700354627H1 | g1707854 | 41 | −48 | gb105pln | T. aestivum mRNA for obtusifoliol 14-alpha-demethylase (CYP51), clone w51, partial. | |
| 700282564H2 | g939780 | 28 | −22 | gb105pln | Zea mays MADS-box protein (ZAG5) mRNA, complete cds. | |
| 700354919H1 | g293888 | 47 | 5 | gb105pln | Zea mays, glyceraldehyde-3-phosphate dehydrogenase mRNA, 3′ end (clone GAPC2). | |
| 700356473H1 | g1237029 | 18 | 5 | gb105allp | alpha-cop protein | |
| 700382560H1 | g1185555 | 24 | −24 | gb105pln | Zea mays glyceraldehyde-3-phosphate dehydrogenase (gpc4) gene, partial cds. | |
| 700382871H1 | g2218151 | 12 | 16 | gb105pln | Vigna unguiculata type IIIa membrane protein cp-wap13 mRNA, complete cds. | |
| 700353952H1 | g1049080 | 14 | 6 | gb105allp | SRp40-1 | |
| 700355756H1 | g1532072 | 43 | −66 | gb105pln | Z. mays mRNA for S-adenosylmethionine decarboxylase. | |
| 700355825H1 | g515758 | 36 | −31 | gb105pln | Vitis vinifera (clone pGME1) malate dehydrogenase mRNA, complete cds. | |
| 700382107H1 | g508543 | 19 | 3 | gb105allp | integral membrane protein 1 | |
| 700354559H1 | g1213460 | 16 | −0 | gb105eukp | C subunit of V-ATPase; EC 3.6.1.35 | |
| 700352842H1 | g2262154 | 39 | −28 | gb105eukp | T10P11.20; predicted protein of unknown function | |
| 700355806H1 | g1788490 | 17 | 4 | gb105allp | hypothetical 32.9 kD protein in nfo-fruA intergenic region | |
| 700355934H1 | g170746 | 77 | −59 | gb105pln | Wheat histone H4 TH091 gene, complete cds. | |
| 700355522H1 | g22423 | 57 | −80 | gb105pln | Z. mays mRNA for polygalacturonase (clone PG4). | |
| 700353789H1 | g22425 | 31 | −30 | gb105pln | Z. mays gene for polygalacturonase (clone 47). | |
| 700355918H1 | g1922313 | 10 | 2 | gb105allp | kinesin-2 | |
| 700382055H1 | g790583 | 37 | −8 | gb105eukp | ATOZI1 | |
| 700355072H1 | g668984 | 48 | −23 | gb105pln | S. tuberosum TYKY1 mRNA for NADH:ubiquinone oxidoreductase. | |
| 700355324H1 | g1289304 | 20 | 7 | gb105eukp | TCP20; Tcp20p | |
| 700352977H1 | g166624 | 29 | 4 | gb105pln | A. thaliana ATPase gene, complete cds. | |
| 700353091H1 | g167107 | 40 | −58 | gb105pln | Hordeum vulgare vacuolar ATPase B subunit isoform mRNA, complete cds. | |
| 700355312H1 | g2828185 | 15 | −17 | gb105pln | Arabidopsis thaliana genomic DNA, chromosome 5, P1 clone: MUD21, complete | |
| | | | | | sequence. | |
| 700357422H1 | g2739367 | 27 | 6 | gb105eukp | T9J22.9; putative phosphatidylinositol-4-phosphate 5-kinase | |
| 700356134H1 | g1167473 | 15 | −0 | gb105eukp | C47E12.8 | |
| 700282529H2 | g1370165 | 34 | −26 | gb105pln | L. japonicus mRNA for small GTP-binding protein, RAB1C. | |
| 700352642H1 | g22277 | 78 | −73 | gb105pln | Maize mRNA for ferritin (clone FM2). | |
| 700354712H1 | g169472 | 23 | −18 | gb105pln | Potato alpha-glucan phosphorylase type H isozyme mRNA, complete cds. | |
| 700352660H1 | g414907 | 27 | −11 | gb105pln | P. pratense mRNA for pollen allergen PhlpI. | |
| 700352793H1 | g2369877 | 66 | −50 | gb105pln | Hordeum vulgare mRNA for m12 protein. | |
| 700352824H1 | g2688979 | 13 | 6 | gb105eukp | AtKUP1; high-affinity potassium transporter | |
| 700355270H1 | g313139 | 47 | 6 | gb105pln | Z. mays ZmPRO2 mRNA for profilin 2. | |
| 700354870H1 | g1015864 | 9 | 4 | gb105eukp | MNS1 | |
| 700282469H2 | g288366 | 62 | −85 | gb105pln | Z. mays PGg14 gene for polygalacturonase. | |
| 700352914H1 | g313137 | 44 | −7 | gb105pln | Z. mays ZmPRO1 mRNA for profilin 1. | |
| 700355426H1 | g496385 | 8 | 3 | gb105eukp | protein kinase | |
| 700355846H1 | g22427 | 58 | −60 | gb105pln | Z. mays gene for polygalacturonase (clone 65). | |
| 700353639H1 | g288611 | 91 | −79 | gb105pln | Z. mays PG gene for polygalacturonase. | |
| 700354467H1 | g459425 | 11 | 12 | gb105pln | B. oleracea L. gene for aspartic protease (partial). | |
| 700353565H1 | g1665777 | 33 | 2 | gb105allp | Similar to S. cerevisiae EMP70 protein precursor (S25110) | |
| 700382607H1 | g1209258 | 20 | 2 | gb105allp | protease inhibitor II | |
| 700356163H1 | g908894 | 6 | 5 | gb105eukp | SPAC13C5.05c; unknown | |
| 700354947H1 | g531031 | 79 | −17 | gb105pln | Pennisetum ciliare apomixis-associated mRNA. | |
| 700354738H1 | g2696803 | 37 | −56 | gb105pln | Oryza sativa mRNA for water channel protein, complete cds. | |
| 700354736H1 | g688011 | 9 | 4 | gb105allp | AgX-1 antigen [human, infertile patient, testis, Peptide, 505 aa] | |
| 700352755H1 | g600768 | 44 | −23 | gb105pln | Oryza sativa cyclophilin 2 (Cyp2) mRNA, complete cds. | |
| 700356230H1 | g2668737 | 59 | −37 | gb105pln | Zea mays translation initiation factor 5A (TIF5A) mRNA, complete cds. | |
| 700354821H1 | g520478 | 15 | 2 | gb105eukp | pyruvate dehydrogenase E1 beta subunit | |
| 700353924H1 | g2264304 | 20 | −11 | gb105pln | Arabidopsis thaliana genomic DNA, chromosome 5, P1 clone: MBG8, complete | |
| | | | | | sequence. | |
| 700356762H1 | g2829910 | 12 | 8 | gb105eukp | F22K20.5 | |
| 700353961H1 | g288373 | 51 | −59 | gb105pln | Z. mays PGg6 gene for polygalacturonase. | |
| 700382168H1 | g1399563 | 40 | −55 | gb105pln | Hydrastis canadensis nuclear 26S ribosomal RNA gene, partial sequence. | |
| 700282529H2 | g303731 | 35 | −26 | gb105pln | Pea mRNA for GTP-binding protein, complete cds. | |
| 700353178H1 | g22458 | 71 | −50 | gb105pln | Z. mays pollen specific mRNA C-terminal (clone 4H7). | |
| 700354941H1 | g435542 | 71 | −52 | gb105pln | Z. mays mRNA for calmodulin. | |
| 700356073H1 | g288373 | 40 | −61 | gb105pln | Z. mays PGg6 gene for polygalacturonase. | |
| 700383118H1 | g2688829 | 31 | −20 | gb105pln | Prunus armeniaca putative sugar transporter mRNA, complete cds. | |
| 700354503H1 | g603848 | 25 | −44 | gb105pln | H. vulgare mRNA for L-Glutamate: tRNA-Glu ligase. | |
| 700383151H1 | g587562 | 30 | −12 | gb105eukp | MPP; mitochondrial processing peptidase | |
| 700282404H2 | g1314064 | 4 | 8 | gb105allp | unknown | |
| 700382383H1 | g1145692 | 30 | −20 | gb105pln | Arabidopsis thaliana actin (ACT1) gene, complete cds. | |
| 700352776H1 | g902583 | 59 | −53 | gb105pln | Zea mays clone MubG1 ubiquitin gene, complete cds. | |
| 700355463H1 | g22427 | 30 | −61 | gb105pln | Z. mays gene for polygalacturonase (clone 65). | |
| 700355459H1 | g2738247 | 53 | −41 | gb105pln | Arabidopsis thaliana cobalamin-independent methionine synthase (ATCIMS) mRNA, | |
| | | | | | complete cds. | |
| 700356858H1 | g1236961 | 10 | −1 | gb105eukp | hydrolysis of the cyanogenic glucoside (R)-prunasin; prunasin hydrolase precursor; | |
| | | | | | EC 3.2.1.21 | |
| 700354412H1 | g22416 | 56 | −17 | gb105pln | Z. mays mRNA for polygalacturonase (clone PG1). | |
| 700382152H1 | g1854550 | 14 | 2 | gb105allp | red-1 | |
| 700356233H1 | g293886 | 87 | −55 | gb105pln | Zea mays glyceraldehyde-3-phosphate dehydrogenase mRNA, 3′ end, (clone GAPC3). | |
| 700382901H1 | g1125691 | 21 | 4 | gb105eukp | dnaJ; DnaJ protein | |
| 700354778H1 | g1438879 | 9 | 1 | gb105eukp | GmCK1p; EC 2.7.1.32 | |
| 700355484H1 | g2599103 | 16 | 10 | gb105pln | Dunaliella salina 60S ribosomal protein (DSRP1) mRNA, complete cds. | |
| 700353109H1 | g1616613 | 40 | −22 | gb105pln | N. plumbaginifolia mRNA for small GTP-binding protein, clone Np43GTP. | |
| 700354287H1 | g2811223 | 36 | −22 | gb105pln | Arabidopsis thaliana fimbrin 1 mRNA, partial cds. | |
| 700353032H1 | g710349 | 41 | −12 | gb105pln | Lupinus albus farnesyl pyrophosphate synthase (fps1) mRNA, complete cds. | |
| 700382064H1 | g2832717 | 28 | −14 | gb105eukp | alinv; sucrose hydrolysis; alkaline/neutral invertase | |
| 700383088H1 | g206436 | 55 | −18 | gb105allp | protstatein C3 subunit | |
| 700282442H2 | g1314049 | 22 | 5 | gb105allp | archain/delta-COP | |
| 700352827H1 | g2598045 | 11 | −3 | gb105eukp | spPMP20; PMP20 | |
| 700382221H1 | g288366 | 75 | −51 | gb105pln | Z. mays PGg14 gene for polygalacturonase. | |
| 700353951H1 | g2316016 | 11 | 6 | gb105allp | MRP-like ABC transporter | |
| 700282510H2 | g22606 | 17 | 7 | gb105pln | H. vulgare mRNA for 14-3-3 protein homologue (14-3-3a). | |
| 700356180H1 | g2384759 | 38 | −16 | gb105pln | Oryza sativa GDP dissociation inhibitor protein OsGDI2 (OsGDI2) mRNA, | |
| | | | | | complete cds. | |
| 700383116H1 | g2160172 | 21 | −12 | gb105eukp | F21M12.20 | |
| 700354224H1 | g22418 | 63 | 5 | gb105pln | Z. mays gene for polygalacturonase (clone 17). | |
| 700282504H2 | g2073489 | 18 | 6 | gb105allp | glutaredoxin like-protein | |
| 700355159H1 | g22456 | 50 | −57 | gb105pln | Z. mays pollen specific mRNA C-terminal (clone 3C12). | |
| 700354405H1 | g2815251 | 22 | 11 | gb105pln | A. thaliana gene for ubiquitin carrier protein. | |
| 700353685H1 | g886401 | 25 | 4 | gb105allp | MADS box protein | |
| 700353180H1 | g1181332 | 43 | −61 | gb105pln | Z. mays CRH mRNA. | |
| 700353068H1 | g899607 | 70 | −61 | gb105pln | Zea mays polyubiquitin (MubC5) mRNA, complete cds. | |
| 700356256H1 | g248336 | 61 | −72 | gb105pln | polyubiquitin [maize, Genomic, 3841 nt]. | |
| 700382107H1 | g624704 | 17 | 4 | gb105allp | putative transmembrane protein precursor | |
| 700353266H1 | g2444419 | 50 | −45 | gb105pln | Glycine max ribosome-associated protein p40 mRNA, complete cds. | |
| 700356544H1 | g2288988 | 26 | 7 | gb105allp | hypothetical protein | |
| 700382804H1 | g2886511 | 51 | −28 | gb105pln | Z. mays PG gene for polygalacturonase. | |
| 700354301H1 | g2244991 | 18 | −33 | gb105pln | Arabidopsis thaliana DNA chromosome 4, ESSA I contig fragment No. 6. | |
| 700355295H1 | g16210 | 52 | 1 | gb105pln | Arabidopsis thaliana calnexin homolog. | |
| 700356028H1 | g2463334 | 59 | −26 | gb105pln | Oryza sativa mRNA for ribosomal protein S4. | |
| 700352674H1 | g18440 | 43 | −30 | gb105pln | Euglena gracilis mRNA for beta-tubulin. | |
| 700352982H1 | g1136121 | 66 | −39 | gb105pln | O. sativa mRNA for alpha-tubulin (clone OSTA-136). | |
| 700383014H1 | g414907 | 22 | −6 | gb105pln | P. pratense mRNA for pollen allergen PhlpI. | |
| 700355407H1 | g2335089 | 11 | 16 | gb105pln | Arabidopsis thaliana chromosome II BAC T11A07 genomic sequence, complete | |
| | | | | | sequence. | |
| 700382376H1 | g857393 | 27 | −4 | gb105eukp | mitotic cyclin a1-type | |
| 700356216H1 | g886130 | 16 | 5 | gb105eukp | putative pectinesterase | |
| 700354738H1 | g1667593 | 36 | −53 | gb105pln | Oryza sativa transmembrane protein mRNA, complete cds. | |
| 700354307H1 | g500750 | 84 | −36 | gb105pln | Zea mays ferredoxin-NADP reductase mRNA, partial cds. | |
| 700354604H1 | g2809258 | 23 | −17 | gb105eukp | F21B7.27 | |
| 700354696H1 | g2154727 | 49 | −35 | gb105pln | C. dactylon mRNA for profilin 2. | |
| 700355413H1 | g22427 | 98 | −87 | gb105pln | Z. mays gene for polygalacturonase (clone 65). | |
| 700354340H1 | g438312 | 13 | 1 | gb105allp | Immune suppressor factor J6B7 precursor | |
| 700356143H1 | g1815759 | 17 | 5 | gb105eukp | pollen-specific protein | |
| 700282438H2 | g2695928 | 18 | 5 | gb105pln | Hordeum vulgare mRNA for putative thiol protease, clone RG42. | |
| 700382789H1 | g22275 | 79 | −72 | gb105pln | Maize mRNA for ferritin (clone FM1). | |
| 700355977H1 | g1747295 | 69 | −56 | gb105pln | Rice mRNA for vacuolar H+-pyrophosphatase, complete cds. | |
| 700382231H1 | g974779 | 21 | −10 | gb105pln | B. vulgaris mRNA for small G protein (clone 1S5). | |
| 700354351H1 | g184657 | 15 | 2 | gb105allp | transfer RNA-Trp synthetase | |
| 700353164H1 | g22174 | 11 | 16 | gb105pln | Maize Aux311 gene for auxin-binding protein. | |
| 700382634H1 | g22514 | 48 | −54 | gb105pln | Maize Zc1 gene for Zein Zc1 (14 kD zein-2). | |
| 700382209H1 | g2275006 | 38 | −31 | gb105pln | Hordeum vulgare mRNA for expressed sequence tag. | |
| 700354874H1 | g248336 | 33 | −49 | gb105pln | polyubiquitin [maize, Genomic, 3841 nt]. | |
| 700354734H1 | g288366 | 76 | −69 | gb105pln | Z. mays PGg14 gene for polygalacturonase. | |
| 700353531H1 | g1805653 | 14 | 11 | gb105pln | B. oleracea BCA1 mRNA for Ca2+-ATPase. | |
| 700353239H1 | g2739370 | 15 | −5 | gb105eukp | T9J22.12; putative pectinesterase | |
| 700382105H1 | g1167835 | 26 | −7 | gb105pln | H. lanatus mRNA for Hol lI protein. | |
| 700354304H1 | g596084 | 22 | −13 | gb105eukp | ribosomal protein YL6 (L5) | |
| 700356575H1 | g2415697 | 18 | −21 | gb105pln | P. pratense mRNA for profilin 2. | |
| 700382373H1 | g1532047 | 15 | 1 | gb105pln | O. sativa mRNA for S-adenosylmethionine decarboxylase. | |
| 700354652H1 | g473359 | 29 | −13 | gb105pln | P. pratense mRNA for Phl pI allergen. | |
| 700355061H1 | g287834 | 48 | −24 | gb105pln | Z. mays yptm2 cDNA. | |
| 700353883H1 | g1209258 | 20 | 2 | gb105allp | protease inhibitor II | |
| 700354553H1 | g1737218 | 56 | −8 | gb105eukp | vacuolar sorting receptor homolog | |
| 700355988H1 | g1039357 | 10 | −5 | gb105eukp | flavonol synthase | |
| 700356270H1 | g1449162 | 28 | −11 | gb105pln | Glycyrrhiza glabra mRNA for squalene synthase, complete cds (GGSQS1). | |
| 700354259H1 | g886470 | 33 | −37 | gb105pln | C. roseus MetE mRNA for methionine synthase. | |
| 700352933H1 | g288373 | 98 | −21 | gb105pln | Z. mays PGg6 gene for polygalacturonase. | |
| 700382895H1 | g1839188 | 17 | −4 | gb105eukp | RHD3; putative GTP-binding protein; root hair defective 3 | |
| 700382972H1 | g313141 | 24 | −30 | gb105pln | Z. mays ZmPRO3 mRNA for profilin 3. | |
| 700382254H1 | g2293565 | 58 | −51 | gb105pln | Oryza sativa ADP-ribosylation factor 1 (Os-ARF1) mRNA, complete cds. | |
| 700354938H1 | g2154727 | 35 | −21 | gb105pln | C. dactylon mRNA for profilin 2. | |
| 700353802H1 | g2351580 | 16 | −8 | gb105eukp | thymidine diphospho-glucose 4-6-dehydratase homolog | |
| 700352707H1 | g736271 | 34 | −50 | gb105pln | O. sativa hsp70 gene for heat shock protein 70. | |
| 700356196H1 | g2384757 | 27 | −9 | gb105pln | Oryza sativa GDP dissociation inhibitor protein OsGDI1 (OsGDI1) mRNA, | |
| | | | | | complete cds. | |
| 700353823H1 | g2446980 | 21 | −7 | gb105pln | Arabidopsis thaliana mRNA for AtGDI2, complete cds. | |
| 700382428H1 | g575354 | 19 | −20 | gb105pln | O. sativa SC34 mRNA for tumor suppressor. | |
| 700355542H1 | g433041 | 18 | −24 | gb105pln | Zea mays W-22 clone PREM-1B retroelement PREM-1, partial sequence. | |
| 700356750H1 | g288366 | 70 | −25 | gb105pln | Z. mays PGg14 gene for polygalacturonase. | |
| 700356606H1 | g1864023 | 48 | −13 | gb105pln | Cynodon dactylon calcium-binding pollen allergen gene, partial cds. | |
| 700355887H1 | g166385 | 29 | −40 | gb105pln | Medicago sativa isocitrate dehydrogenase mRNA, 3′ end cds. | |
| 700356223H1 | g21629 | 36 | −54 | gb105pln | Sorghum vulgare mRNA for phosphoenolpyruvate carboxylase (PEPC). | |
| 700357439H1 | g2645162 | 34 | 4 | gb105pln | Oryza sativa mRNA, similar to initiation factor eIF-4c. | |
| 700354379H1 | g1184771 | 80 | −73 | gb105pln | Zea mays glyceraldehyde-3-phosphate dehydrogenase GAPC2 (gpc2) mRNA, | |
| | | | | | complete cds. | |
| 700356123H1 | g414907 | 28 | −14 | gb105pln | P. pratense mRNA for pollen allergen PhlpI. | |
| 700382263H1 | g473359 | 32 | −7 | gb105pln | P. pratense mRNA for PhlpI allergen. | |
| 700352940H1 | g17561 | 16 | 8 | gb105allp | PROTEINASE INHIBITOR | |
| 700354961H1 | g1173557 | 26 | 5 | gb105allp | Ory s 1 | |
| 700356193H1 | g2828188 | 19 | 5 | gb105pln | Arabidopsis thaliana genomic DNA, chromosome 5, TAC clone: K3I3, complete | |
| | | | | | sequence. | |
| 700355590H1 | g2384757 | 34 | −17 | gb105pln | Oryza sativa GDP dissociation inhibitor protein OsGDI1 (OsGDI1) mRNA, | |
| | | | | | complete cds. | |
| 700354469H1 | g313759 | 55 | −41 | gb105pln | Z. mays hsp 70-1 gene for heat shock protein 70. | |
| 700354922H1 | g2213582 | 7 | 7 | gb105eukp | T7N9.2 | |
| 700353531H1 | g509809 | 16 | −3 | gb105pln | Arabidopsis thaliana chloroplast envelope Ca2+-ATPase (PEA1) mRNA, complete cds. | |
| 700352617H1 | g453975 | 41 | −42 | gb105pln | P. pratense mRNA for profilin-Phl p XI. | |
| 700382820H1 | g562074 | 14 | 3 | gb105allp | ribosomal protein L35 | |
| 700354945H1 | g1184773 | 62 | −44 | gb105pln | Zea mays glyceraldehyde-3-phosphate dehydrogenase GAPC3 (gpc3) mRNa, | |
| | | | | | complete cds. | |
| 700356777H1 | g450548 | 65 | −22 | gb105pln | O. sativa (pRSAM-1) gene for S-adenosyl methionine synthetase. | |
| 700382376H1 | g1665741 | 25 | −4 | gb105eukp | cyclin | |
| 700356248H1 | g248338 | 62 | −22 | gb105pln | polyubiquitin [maize, Genomic, 3439 nt]. | |
| 700356747H1 | g21599 | 37 | 1 | gb105eukp | UTP-glucose-1-phosphate uridylyltransferase; EC 2.7.7.9 | |
| 700354570H1 | g19501 | 33 | 1 | gb105eukp | pPLZ12 gene product (AA 1-184) | |
| 700352888H1 | g1872523 | 36 | −8 | gb105eukp | LSD1; negative regulator of cell death; zinc-finger protein Lsd1 | |
| 700354338H1 | g169898 | 11 | 7 | gb105eukp | 28 kDa protein | |
| 700382068H1 | g2494106 | 11 | 7 | gb105pln | Arabidopsis thaliana chromosome 1 YAC yUP8H12R, complete sequence. | |
| 700382173H1 | g288378 | 97 | −79 | gb105pln | Z. mays gene PG for polygalacturonase. | |
| 700355854H1 | g550306 | 6 | 7 | gb105eukp | ATPME1 | |
| 700356643H1 | g288611 | 48 | −30 | gb105pln | Z. mays PG gene for polygalacturonase. | |
| 700382758H1 | g1707639 | 70 | −64 | gb105pln | O. sativa mRNA for transcription activator, GAMyb. | |
| 700382237H1 | g1906830 | 20 | 1 | gb105eukp | hsp88.1; heat shock protein | |
| 700352923H1 | g288373 | 100 | −57 | gb105pln | Z. mays PGg6 gene for polygalacturonase. | |
| 700382818H1 | g633135 | 39 | −30 | gb105pln | Zostera marina mRNA for putative plasma membrane H+-ATPase, complete cds. | |
| 700355414H1 | g1835730 | 17 | −5 | gb105pln | Oryza sativa photosystem II 10 kDa polypeptide mRNa, complete cds. | |
| 700354324H1 | g2505865 | 13 | −11 | gb105eukp | putative topoisomerase | |
| 700352677H1 | g303852 | 63 | −53 | gb105pln | Rice mRNa for ribosomal protein L3, complete cds. | |
| 700356611H1 | g1051257 | 55 | −12 | gb105pln | Hordeum vulgare vacuolar ATPase catalytic subunit mRNA, partial cds. | |
| 700354856H1 | g1388021 | 19 | −6 | gb105eukp | UGPase; UDP-glucose pyrophosphorylase; EC 2.7.7.9 | |
| 700356819H1 | g1679760 | 10 | 3 | gb105eukp | chk-1; two-component histidine kinase CHK-1 | |
| 700354681H1 | g313137 | 48 | −28 | gb105pln | Z. mays ZmPRO1 mRNA for profilin 1. | |
| 700382283H1 | g2317906 | 10 | 6 | gb105eukp | T7I23.6; ARA-5 | |
| 700353045H1 | g1419367 | 23 | −20 | gb105pln | Z. mays ZmABP2 mRNA for actin depolymerizing factor. | |
| 700356413H1 | g1899026 | 28 | −37 | gb105pln | Zea mays superoxide dismutase 4A (sod4A) gene, complete cds. | |
| 700354878H1 | g2661608 | 16 | −1 | gb105eukp | SPAC10F6.03c; hypothetical ctp synthase | |
| 700353643H1 | g872315 | 8 | 6 | gb105allp | 40S ribosomal protein S12 | |
| 700356468H1 | g498745 | 12 | 6 | gb105eukp | PIR7b; Pir7b | |
| 700354304H1 | g599981 | 22 | −13 | gb105eukp | putative 60S ribosomal protein | |
| 700282455H2 | g22427 | 52 | −80 | gb105pln | Z. mays gene for polygalacturonase (clone 65). | |
| 700353260H1 | g2262173 | 19 | −4 | gb105eukp | F5J6.18; NADPH thioredoxin reductase | |
| 700356666H1 | g1777706 | 62 | −27 | gb105pln | Zea mays 18S ribosomal RNA gene, partial sequence. | |
| 700354754H1 | g460042 | 27 | −7 | gb105eukp | DBF3; involved in the control of S phase and pre-mRNA splicing | |
| 700382546H1 | g416144 | 50 | −27 | gb105pln | Zea mays beta-4 tubulin (tub4) mRNA, complete cds. | |
| 700382181H1 | g2145472 | 34 | −16 | gb105pln | S. tuberosum mRNA for aconitase/aconitate hydratase. | |
| 700354787H1 | g2464934 | 23 | −6 | gb105eukp | serine C-palmitoyltransferase homolog | |
| 700354280H1 | g22420 | 73 | −57 | gb105pln | Z. mays mRNA for polygalacturonase (clone PG2). | |
| 700352970H1 | g1279254 | 33 | −8 | gb105eukp | C35A5.9 | |
| 700355701H1 | g287829 | 41 | −11 | gb105pln | Z. mays gene for polygalacturonase. | |
| 700354822H1 | g1483150 | 7 | 5 | gb105eukp | monodehydroascorbate reductase | |
| 700355950H1 | g2583106 | 16 | 10 | gb105pln | Arabidopsis thaliana chromosome II BAC F4L23 genomic sequence, complete sequence. | |
| 700352694H1 | g288373 | 74 | −66 | gb105pln | Z. mays PGg6 gene for polygalacturonase. | |
| 700357140H1 | g22062 | 21 | −7 | gb105eukp | sulfydryl-pre-endopeptidase (AA −20 to 342) | |
| 700353620H1 | g168423 | 87 | −86 | gb105pln | Zea mays polypeptide chain-binding protein mRNa, 3′ end. | |
| 700352788H1 | g2791948 | 15 | −0 | gb105eukp | rp113a; ribosomal protein L13a | |
| 700354960H1 | g1666175 | 54 | 5 | gb105allp | ubiquitin | |
| 700354636H1 | g2618602 | 11 | 13 | gb105pln | Arabidopsis thaliana genomic DNA, chromosome 5, P1 clone: MSJ1, complete | |
| | | | | | sequence. | |
| 700382804H1 | g288378 | 36 | −15 | gb105pln | Z. mays gene PG for polygalacturonase. | |
| 700355188H1 | g1498052 | 94 | −86 | gb105pln | Zea mays ribosomal protein S8 mRNA, complete cds. | |
| 700355978H1 | g1813595 | 14 | −2 | gb105eukp | xylan endohydrolase isoenzyme X-I; EC 3.2.1.8 | |
| 700383195H1 | g531757 | 14 | 11 | gb105pln | Yeast (Saccharomyces cerevisiae) YTA5 gene. | |
| 700382512H1 | g289212 | 23 | −4 | gb105pln | Atriplex nummularia homologous sequence (ANJ1) mRNA. | |
| 700353565H1 | g2814799 | 29 | 4 | gb105eukp | ZK858.6 | |
| 700382034H1 | g601789 | 23 | −11 | gb105eukp | AFC2; protease kinase | |
| 700352909H1 | g429148 | 43 | −18 | gb105pln | Z. mays pep gene for (C3 type) phosphoendopyruvate carboxylase. | |
| 700353867H1 | g17789 | 15 | −2 | gb105eukp | Bp10 | |
| 700353165H1 | g2661840 | 14 | −6 | gb105eukp | adk; adenosine kinase; EC 2.7.1.20 | |
| 700355238H1 | g2252639 | 14 | −2 | gb105pln | Genomic sequence of Arabidopsis BAC F8A5, complete sequence. | |
| 700352855H1 | g16119 | 23 | −36 | gb105pln | Oat spe1 mRNA for arginine decarboxylase (EC 4.1.1.19). | |
| 700383102H1 | g288611 | 60 | −49 | gb105pln | Z. mays PG gene for polygalacturonase. | |
| 700382091H1 | g287830 | 17 | −8 | gb105eukp | polygalacturonase | |
| 700355401H1 | g409072 | 7 | 5 | gb105allp | HBp15/L22 | |
| 700383152H1 | g22324 | 30 | −38 | gb105pln | Z. mays mRNA for H2B histone (clone cH2B2210). | |
| 700382658H1 | g722325 | 53 | −68 | gb105pln | Zea mays clone Zm-Rab2-A GTP binding protein (rab2) mRNA, complete cds. | |
| 700355346H1 | g2624199 | 18 | −5 | gb105pln | M. acuminata mRNA; clone pBAN UU93. | |
| 700354941H1 | g2809480 | 54 | −36 | gb105pln | Oryza sativa calmodulin (CaM2) mRNA, complete cds. | |
| 700282526H2 | g166581 | 22 | −7 | gb105pln | A. thaliana AAc1 gene encoding actin-1, complete cds. | |
| 700355411H1 | g397958 | 40 | −69 | gb105pln | Z. mays mRNA for beta-amylase. | |
| 700354828H1 | g22456 | 50 | −52 | gb105pln | Z. mays pollen specific mRNA C-terminal (clone 3C12). | |
| 700354737H1 | g603280 | 9 | 2 | gb105eukp | YER047C; Yer047cp | |
| 700354387H1 | g1143156 | 14 | −4 | gb105eukp | acyl-ACP thioesterase | |
| 700354363H1 | g499011 | 56 | −46 | gb105pln | S. vulgare SoAc1 mRNA. | |
| 700353209H1 | g1246115 | 21 | 1 | gb105pln | major allergen Pha a1 [Phalaris aquatica = canary grass, pollen, mRNA Partial, | |
| | | | | | 1088 nt]. | |
| 700282578H2 | g22243 | 13 | 4 | gb105pln | Zea mays Cin1 repeat from Cin1 middle repetitive family. | |
| 700355035H1 | g602564 | 32 | −1 | gb105pln | C. paradisi (Macf) INO1 gene. | |
| 700355748H1 | g436051 | 38 | −39 | gb105pln | Rice mRNA for branching enzyme-3, complete cds. | |
| 700356174H1 | g1750379 | 28 | −6 | gb105pln | Eucalyptus globulus NADP-isocitrate dehydrogenase (EgICDH) mRNA, complete cds. | |
| 700353917H1 | g22416 | 64 | 1 | gb105pln | Z. mays mRNA for polygalacturonase (clone Pg1). | |
| 700382241H1 | g2191175 | 11 | 2 | gb105allp | No definition line found | |
| 700352764H1 | g600768 | 52 | −38 | gb105pln | Oryza sativa cyclophilin 2 (Cyp2) mRNA, complete cds. | |
| 700353025H1 | g22606 | 33 | −31 | gb105pln | H. vulgare mRNA for 14-3-3 protein homologue (14-3-3a). | |
| 700353019H1 | g16427 | 16 | 8 | gb105eukp | protease inhibitor II | |
| 700356564H1 | g20321 | 45 | −48 | gb105pln | Oryza sativa RAc1 mRNA for actin. | |
| 700355719H1 | g1817548 | 15 | 4 | gb105allp | phosphoethanolamine citidylyltransferase | |
| 700352834H1 | g2342717 | 15 | 16 | gb105pln | Arabidopsis thaliana chromosome II BAC T14G11 genomic sequence, complete | |
| | | | | | sequence. | |
| 700353151H1 | g21479 | 24 | −3 | gb105eukp | hsp70; 70-Kd heat shock protein | |
| 700357145H1 | g2431770 | 92 | −88 | gb105pln | Zea mays acidic ribosomal protein P2b (rpp2b) mRNA, complete cds. | |
| 700353954H1 | g1052973 | 34 | −13 | gb105eukp | fructokinase; EC 2.7.1.4 | |
| 700357143H1 | g288366 | 76 | −72 | gb105pln | Z. mays PGg14 gene for polygalacturonase. | |
| 700355236H1 | g2827079 | 62 | −3 | gb105pln | Medicago sativa mitochondrial malate dehydrogenase precursor (mmdh) mRNA, nuclear | |
| | | | | | gene encoding mitochondrial protein, complete cds. | |
| 700355307H1 | g2351070 | 13 | 12 | gb105pln | Arabidopsis thaliana genomic DNA, chromosome 5, P1 clone: MTH12. | |
| 700355958H1 | g20321 | 72 | −62 | gb105pln | Oryza sativa RAc1 mRNA for actin. | |
| 700382325H1 | g2425100 | 13 | 12 | gb105pln | Oryza sativa cationic peroxidase (OsCPX1) mRNA, complete cds. | |
| 700352955H1 | g1165139 | 74 | −19 | gb105allp | ribosomal protein L4 | |
| 700356415H1 | g169481 | 15 | 7 | gb105eukp | calcium-binding protein | |
| 700354943H1 | g1314090 | 15 | 6 | gb105allp | unknown | |
| 700356472H1 | g2351066 | 30 | 4 | gb105pln | Arabidopsis thaliana genomic DNA, chromosome 5, P1 clone: MOP9. | |
| 700354719H1 | g2370497 | 7 | −3 | gb105eukp | SPAC25H1.08c; beta transducin | |
| 700355191H1 | g1732518 | 32 | −12 | gb105pln | Arabidopsis thaliana Rho1Ps homolog mRNA, complete cds. | |
| 700354407H1 | g2341032 | 12 | −5 | gb105eukp | F19P19.11 | |
| 700355982H1 | g313151 | 26 | −10 | gb105pln | A. thaliana ribosomal protein S15 mRNA, complete cds. | |
| 700352708H1 | g1789489 | 10 | −1 | gb105allp | hypothetical 37.4 kD protein in exuR-tdcC intergenic region | |
| 700382886H1 | g22427 | 48 | −59 | gb105pln | Z. mays gene for polygalacturonase (clone 65). | |
| 700355542H1 | g433037 | 19 | −29 | gb105pln | Zea mays W-22 clone cDNA4 retroelement PREM-1-containing mRNA. | |
| 700357165H1 | g899607 | 56 | −53 | gb105pln | Zea mays polyubiquitin (MubC5) mRNA, complete cds. | |
| 700353072H1 | g2394434 | 21 | −4 | gb105eukp | F23F1.8 | |
| 700352759H1 | g2431768 | 35 | −29 | gb105pln | Zea mays acidic ribosomal protein P1a (rpp1a) mRNA, complete cds. | |
| 700354866H1 | g287829 | 31 | −35 | gb105pln | Z. mays gene for polygalacturonase. | |
| 700353844H1 | g22458 | 27 | −21 | gb105pln | Z. mays pollen specific mRNA C-terminal (clone 4H7). | |
| 700355829H1 | g168557 | 44 | 4 | gb105pln | Zea mays putative GTP-binding protein homolog mRNA, partial cds. | |
| 700355812H1 | g22427 | 70 | −88 | gb105pln | Z. mays gene for polygalacturonase (clone 65). | |
| 700354918H1 | g287830 | 12 | 7 | gb105eukp | polygalacturonase | |
| 700357449H1 | g2443388 | 65 | −6 | gb105eukp | calcium-dependent protein kinase-related kinase | |
| 700354517H1 | g506792 | 26 | 6 | gb105allp | Sec61 protein complex gamma subunit | |
| 700355573H1 | g643596 | 88 | −84 | gb105pln | Corn mRNA for cysteine proteinase, clone CCP, complete cds. | |
| 700382866H1 | g1173555 | 35 | −14 | gb105eukp | galE; catabolism of galactose to glucose in Leloir pathway, and in galactose synthesis | |
| | | | | | from glucose.; UDP-galactose-4-epimerase; EC 5.1.3.2 | |
| 700282485H2 | g2739020 | 11 | 17 | gb105pln | Rhododendron metternichii var. hondoense CT, AT, GT repeat, genomic sequence. | |
| 700382688H1 | g2266994 | 9 | 2 | gb105allp | O-linked GlcNAc transferase | |
| 700355467H1 | g1246887 | 8 | 6 | gb105allp | alpha-L-arabinofuranosidase A | |
| 700356285H1 | g1498241 | 21 | −7 | gb105eukp | MRF1 | |
| 700382518H1 | g22314 | 32 | 2 | gb105pln | Maize mRNA for GSH gluthathione S-transferase I (GST; EC 2.5.1.18). | |
| 700382730H1 | g1469809 | 23 | −4 | gb105allp | nuclear protein, NP220 | |
| 700282516H2 | g166581 | 13 | −3 | gb105pln | A. thaliana AAc1 gene encoding actin-1, complete cds. | |
| 700352942H1 | g1769965 | 36 | −18 | gb105pln | B. napus mRNA for enoyl reductase. | |
| 700353071H1 | g1848211 | 38 | −20 | gb105pln | N. tabacum mRNA for protein disulfide-isomerase precursor. | |
| 700352952H1 | g288373 | 100 | −87 | gb105pln | Z. mays PGg6 gene for polygalacturonase. | |
| 700356833H1 | g1888359 | 19 | −13 | gb105eukp | KAS III; 3-ketoacyl-acyl carrier protein synthase III | |
| 700356253H1 | g21624 | 26 | 13 | gb105pln | S. vulgare mRNA for glycine-rich RNA-binding protein (clone S2). | |
| 700382512H1 | g1872162 | 17 | 2 | gb105pln | Arabidopsis thaliana DnaJ homolog (atj) mRNA, complete cds. | |
| 700382412H1 | g22421 | 92 | −16 | gb105pln | Z. mays mRNA for polygalacturonase (clone PG3). | |
| 700382473H1 | g1669599 | 15 | −4 | gb105eukp | AR791 | |
| 700355867H1 | g1707854 | 57 | −51 | gb105pln | T. aestivum mRNA for obtusifoliol 14-alpha-demethylase (CYP51), clone w51, partial. | |
| 700355844H1 | g2292907 | 14 | −4 | gb105eukp | P-glycoprotein homologue | |
| 700353047H1 | g459197 | 60 | −19 | gb105pln | Gossypium hirsutum vacuolar H+-ATPase subunit B mRNA, complete cds. | |
| 700356193H1 | g1518539 | 27 | −3 | gb105pln | Glycine max UDP-glucose dehydrogenase mRNA, complete cds. | |
| 700355413H1 | g22456 | 100 | −86 | gb105pln | Z. mays pollen specific mRNA C-terminal (clone 3C12). | |
| 700356814H1 | g1167835 | 27 | −23 | gb105pln | H. lanatus mRNA for Hol lI protein. | |
| 700353917H1 | g22421 | 64 | 1 | gb105pln | Z. mays mRNA for polygalacturonase (clone PG3). | |
| 700354271H1 | g22426 | 16 | −0 | gb105eukp | polygalacturonase | |
| 700354577H1 | g1777413 | 35 | 3 | gb105pln | Aspergillus niger 26S proteasome subunit (tbpA) gene, complete cds. | |
| 700353524H1 | g22636 | 42 | 7 | gb105eukp | 70 kDa heat shock protein | |
| 700355436H1 | g1184771 | 65 | −73 | gb105pln | Zea mays glyceraldehyde-3-phosphate dehydrogenase GAPC2 (gpc2) mRNA, | |
| | | | | | complete cds. | |
| 700354663H1 | g21953 | 23 | 3 | gb105eukp | Li; beta-glucosidase; EC 3.2.1.21; linamarase | |
| 700353620H1 | g1575127 | 89 | −86 | gb105pln | Zea mays lumenal binding protein cBiPe2 mRNA, complete cds. | |
| 700282489H2 | g857573 | 33 | 2 | gb105pln | Oryza sativa vacuolar H+-ATPase (vatp-P1) mRNA, complete cds. | |
| 700355723H1 | g10061 | 25 | −17 | gb105eukp | ribosomal protein; L21 | |
| 700356643H1 | g288378 | 38 | −13 | gb105pln | Z. mays gene PG for polygalacturonase. | |
| 700356133H1 | g2827631 | 32 | −22 | gb105eukp | F10N7.130; putative protein | |
| 700382385H1 | g397632 | 63 | −50 | gb105pln | T. aestivum translation initiation factor 4A. | |
| 700354263H1 | g439482 | 32 | −22 | gb105pln | Cucumber mRNA for ferrochelatase. | |
| 700383058H1 | g2230959 | 21 | −12 | gb105eukp | P69B; subtilisin-like protease | |
| 700382235H1 | g313139 | 47 | −37 | gb105pln | Z. mays ZmPRO2 mRNA for profilin 2. | |
| 700354919H1 | g1185553 | 47 | 6 | gb105pln | Zea mays glyceraldehyde-3-phosphate dehydrogenase (gpc2) gene, complete cds. | |
| 700353105H1 | g2827551 | 9 | 4 | gb105eukp | T12H17.130; predicted protein | |
| 700355467H1 | g567830 | 8 | 6 | gb105eukp | abfA; alpha-L-arabinofuranosidase; EC 3.2.1.55 | |
| 700354333H1 | g2347119 | 38 | −25 | gb105eukp | nDiTPx; thioredoxin peroxidase | |
| 700353820H1 | g2668740 | 22 | 3 | gb105eukp | TIF; translation initiation factor | |
| 700355961H1 | g21190 | 81 | −16 | gb105pln | Secale cereale 5 S rRNa. | |
| 700382112H1 | g436051 | 30 | −57 | gb105pln | Rice mRNA for branching enzyme-3, complete cds. | |
| 700356125H1 | g2641618 | 76 | −66 | gb105pln | Zea mays ubiquitin-conjugating enzyme protein E2 (ubc7) mRNA, complete cds. | |
| 700382933H1 | g1651459 | 5 | 6 | gb105allp | Aminopeptidase N | |
| 700382882H1 | g2331140 | 17 | 8 | gb105pln | Oryza sativa water-stress inducible protein (WSI) mRNA, complete cds. | |
| 700355501H1 | g20323 | 25 | −32 | gb105pln | O. sativa RAc1 gene for actin. | |
| 700354265H1 | g1354863 | 24 | −4 | gb105eukp | COI; cytochrome oxidase | |
| 700355062H1 | g603997 | 21 | 7 | gb105pln | Yeast (Saccharomyces cerevisiae) chromosome IX complete sequence. | |
| 700356442H1 | g288366 | 100 | −60 | gb105pln | Z. mays PGg14 gene for polygalacturonase. | |
| 700354856H1 | g218001 | 19 | −6 | gb105eukp | UDP-glucose pyrophosphorylase precursor; EC 2.7.7.9 | |
| 700353074H1 | g434327 | 57 | −53 | gb105pln | Z. mays mRNA gs1-3 for glutamine synthetase. | |
| 700353218H1 | g2804258 | 19 | −18 | gb105eukp | phosphoglycerate dehydrogenase | |

TABLE 2
UNIQUE AND HOMOLOGOUS CLUSTERS IN SATMON021024
COPYRIGHT 1998 INCYTE PHARMACEUTICALS, INC.

Cluster number: 69
Cluster clones: 700342711H1 700343915H1 700344781H1 700344461H1 700344178H1
700342976H1 700346893H1 700345950H1 700347239H1 700345988H1 700342385H1
Percent of clones expressed: 0.87
Homologous match to
g1632830 R. communis mRNA (unknown).
Cluster number: 70
Cluster clones: 700344704H1 700346502H1 700346702H1 700380601H1 700380774H1
700342388H1 700345470H1 700343568H1 700346695H1 700346233H1
Percent of clones expressed: 0.79
Cluster number: 264
Cluster clones: 700345510H1 700343780H1 700345052H1 700344946H1 700344949H1
700344870H1 70034703651 700347281H1
Percent of clones expressed: 0.63
Cluster number: 251
Cluster clones: 700345605H1 700346889H1 700343657H1 700379655H1 700347017H1
700347163H1 700344976H1 700380110H1
Percent of clones expressed: 0.63
Homologous match to
g2407800 H2Av; histone H2A.F/Z
Cluster number: 258
Cluster clones: 700345963H1 700344237H1 70034565051 700344480H1 700343713H1
700346641H1 700345630H1 700345242H1
Percent of clones expressed: 0.63
Homologous match to
g2276349 Yeast (Saccharomyces pombe) chromosome II cosmid c30D10.
Cluster number: 150
Cluster clones: 700345233H1 700343424H1 700380681H1 700343761H1 700346147H1
700343655H1 700345273H1 700342828H1
Percent of clones expressed: 0.63
Homologous match to
g2414643 Yeast (Saccharomyces pombe) chromosome I cosmid c3H5.
Cluster number: 29
Cluster clones: 700343054H1 700345546H1 700343480H1 700347111H1
700281968H2 700346527H1 700345772H1
Percent of clones expressed: 0.55
Homologous match to
g458972 F37C12.4
cluster number: 37
Cluster clones: 700345749H1 700342268H1 700342223H1 700346721H1 700345477H1
700343040H1 700380603H1
Percent of clones expressed: 0.55
Weak match to
g532097 weakly similar to S. cerevisiae hypothetical 24.1 kd protein in
APN1 3′ region
Cluster number: 52
Cluster clones: 700344416H1 700344369H1 700342295H1 700379675H1 700345556H1
700342395H1
Percent of clones expressed: 0.48
Homologous match to
g971699 ribosomal protein S7
Cluster number: 50
Cluster clones: 700344525H1 700342790H1 700379867H1 700342291H1 700342521H1
Percent of clones expressed: 0.40
Cluster number: 254
Cluster clones: 700344013H1 700343679H1 700346877H1 700380604H1 700343721H1
Percent of clones expressed: 0.40
Cluster number: 302
Cluster clones: 700347033H1 700345017H1 700344273H1 70034H121H1 700347383H1
Percent of clones expressed: 0.40
Homologous match to

| | |
|---|---|
| g1388084 | TRX5; thioredoxin h |
| Cluster number: 15 | |
| Cluster clones: 700342656H1 700346909H1 700347166H1 700343904H1 | |
| 700281843H2 | |
| Percent of clones expressed: 0.40 | |
| Homologous match to | |
| g313151 A. thaliana ribosomal protein S15 mRNA, complete CDS. | |
| Cluster number: 222 | |
| Cluster clones: 700344093H1 700343401H1 700346896H1 700346053H1 700346364H1 | |
| Percent of clones expressed: 0.40 | |
| Homologous match to | |
| g310322 Oryza sativa triosephosphate isomerase (Rictipi2) gene, exons 1-9. | |
| Cluster number: 129 | |
| Cluster clones: 700344654H1 700347193H1 700345675H1 700345743H1 700342679H1 | |
| Percent of clones expressed: 0.40 | |
| Cluster number: 166 | |
| Cluster clones: 700346429H1 700346004H1 700342935H1 700347047H1 700379632H1 | |
| Percent of clones expressed: 0.40 | |
| Homologous match to | |
| g2661840 adk; adenosine kinase; EC 2.7.1.20 | |
| Cluster number: 202 | |
| Cluster clones: 700346101H1 700345431H1 700346748H1 700343219H1 | |
| Percent of clones expressed: 0.32 | |
| Homologous match to | |
| g551482 Zea mays ABA- and ripening-inducible-like protein mRNA, complete | |
| cds. | |
| Cluster number: 26 | |
| Cluster clones: 700342283H1 700380411H1 700390596H1 | |
| 700281941H2 | |
| Percent of clones expressed: 0.32 | |
| Weak match to | |
| g2583081 microsomal glutathione S-transferase 3 | |
| Cluster number: 400 | |
| Cluster clones: 700380405H1 700347043H1 700347339H1 700347112H1 | |
| Percent of clones expressed: 0.32 | |
| Cluster number: 20 | |
| Cluster clones: 700343752H1 | |
| 700281888H2 700346930H1 700343674H1 | |
| Percent of clones expressed: 0.32 | |
| Weak match to | |
| g2827559 T12H17.210; predicted protein | |
| Cluster number: 44 | |
| Cluster clones: 700342263H1 700346807H1 700344069H1 700346291H1 | |
| Percent of clones expressed: 0.32 | |
| Cluster number: 7 | |
| Cluster clones: 700343173H1 700345245H1 | |
| 700281819H2 700345179H1 | |
| Percent of clones expressed: 0.32 | |
| Homologous match to | |
| g438279 Ribosomal protein L7 | |
| Cluster number: 117 | |
| Cluster clones: 700343247H1 700344866H1 700342630H1 700343872H1 | |
| Percent of clones expressed: 0.32 | |
| Cluster number: 354 | |
| Cluster clones: 700345561H1 700380563H1 700347003H1 700346226H1 | |
| Percent of clones expressed: 0.32 | |
| Homologous match to | |
| g1321660 Rice mRNA for ascorbate peroxidase, complete cds. | |
| Cluster number: 188 | |
| Cluster clones: 700345696H1 700343091H1 700346267H1 700380511H1 | |
| Percent of clones expressed: 0.32 | |
| Homologous match to | |
| g533689 Brassica napus Naehan RNA polymerase II subunit RPB10 homolog | |
| (BRP10) mRNA, complete cds. | |
| Cluster number: 185 | |
| Cluster clones: 700343202H1 700379793H1 700345638H1 700343071H1 | |
| Percent of clones expressed: 0.32 | |
| Weak match to | |
| g19867 extensin (AA 1-620) | |
| Cluster number: 216 | |
| Cluster clones: 700344234H1 700347371H1 700343374H1 700344184H1 | |
| Percent of clones expressed: 0.32 | |
| Homologous match to | |
| g2529657 Arabidopsis theliana chromosome II BAC T301122 genomic sequence, | |
| complete sequence. | |
| Cluster number: 164 | |
| Cluster clones: 7003429241H1 700346794H1 700343408H1 700344614H1 | |
| Percent of clones expressed: 0.32 | |
| Cluster number: 304 | |
| Cluster clones: 700379957H1 700344316H1 700344456H1 700344389H1 | |
| Percent of clones expressed: 0.32 | |
| Homologous match to | |
| g940843 orf 06116 | |
| Cluster number: 303 | |
| Cluster clones: 700346221H1 700347168H1 700344314H1 700345111H1 | |
| Percent of clones expressed: 0.32 | |
| Homologous match to | |
| g2160155 Sequence of BAC F21M12 from Arabidopsis thaliana chromosome 1, | |
| complete sequence. | |
| Cluster number: 105 | |
| Cluster clones: 700379754H1 700342557H1 700344522H1 700380186H1 | |
| Percent of clones expressed: 0.32 | |
| Homologous match to | |
| g218176 Rice mRNA for 2lkd polypeptide. | |
| Cluster number: 269 | |
| Cluster clones: 700345844H1 700343818H1 700380531H1 700379985H1 | |
| Percent of clones expressed: 0.32 | |
| Homologous match to | |
| g218160 Oryza sativa mRNA for elongation factor 1 beta′. | |
| Cluster number: 358 | |
| Cluster clones: 700347158H1 700346795H1 700380779H1 700345561H1 | |
| Percent of clones expressed: 0.32 | |
| Homologous match to | |
| g857573 Oryza sativa vacuolar H+-ATPase (vatp-P1) mRNA, complete ods. | |
| Cluster number: 11 | |
| Cluster clones: | |
| 700281992112 700346681H1 700344941H1 | |
| 700281833H2 | |
| Percent of clones expressed: 0.32 | |
| Homologous match to | |
| g575730 Z. mays mRNA for transmembrane protein. | |
| Cluster number: 229 | |
| Cluster clones: 700343461H1 700346979H1 700345232H1 700346269H1 | |
| Percent of clones expressed: 0.32 | |
| Homologous match to | |
| g161631 217 g protein | |
| Cluster number: 248 | |
| Cluster clones: 700345114H1 700343626H1 700343695H1 700346604H1 | |
| Percent of clones expressed: 0.32 | |
| Cluster number: 21 | |
| Cluster clones: | |
| 700281910H1 700343988H1 700342664H1 700342817H1 | |
| Percent of clones expressed: 0.32 | |
| Weak match to | |
| g313148 MSK-3; protein kinase | |
| Cluster number: 133 | |
| Cluster clones: 700342705H1 700346924H1 700344370H1 | |
| Percent of clones expressed: 0.24 | |
| Homologous match to | |
| g1778373 Pisum sativum glycine-rich RNA-binding protein PsGRBP mRNA, | |
| complete cds. | |
| Cluster number: 114 | |
| Cluster clones: 700345742H1 700342604H1 700346891H1 | |
| Percent of clones expressed: 0.24 | |
| Homologous match to | |
| g2760330 F1N21.15 | |
| Cluster number: 320 | |
| Cluster clones: 700344853H1 700345514H1 700346207H1 | |
| Percent of clones expressed: 0.24 | |
| Homologous match to | |
| g304153 sorbitol dehydrogenase | |
| Cluster number: 228 | |
| Cluster clones: 700343443H1 700344968H1 700347375H1 | |
| Percent of clones expressed: 0.24 | |
| Homologous match to | |
| g1165205 Arabidopsis thaliana multiubiquitin chain binding protein (MBP1) | |
| mRNA, complete cds. | |
| Cluster number: 209 | |
| Cluster clones: 700343281H1 700344105H1 700347368H1 | |
| Percent of clones expressed: 0.24 | |
| Weak match to | |
| g2370518 ribosomal protein | |
| Cluster number: 6 | |
| Cluster clones: 700345253H1 | |
| 700281814H2 700345138H1 | |
| Percent of clones expressed: 0.24 | |
| Cluster number: 340 | |
| Cluster clones: 700345916H1 700345263H1 700345849H1 | |
| Percent of clones expressed: 0.24 | |
| Cluster number: 67 | |
| Cluster clones: 700345394H1 700344820H1 700342373H1 | |
| Percent of clones expressed: 0.24 | |
| Cluster number: 408 | |
| Cluster clones: 700380484H1 700380031H1 700380409H1 | |
| Percent of clones expressed: 0.24 | |
| Homologous match to | |
| g304108 Arabidopsis thaliana poly(A) -binding protein mRNA, complete cds. | |
| Cluster number: 76 | |
| Cluster clones: 700342419H1 700345701H1 700342873H1 | |
| Percent of clones expressed: 0.24 | |
| Homologous match to | |
| g432367 Rice mRNA for elongation factor 1 beta, complete cds. | |
| Cluster number: 81 | |
| Cluster clones: 700342448H1 700344208H1 700344059H1 | |
| Percent of clones expressed: 0.24 | |
| Weak match to | |
| g22317 GST III (AA 1-222) | |
| Cluster number: 162 | |
| Cluster clones: 700344935H1 700342903H1 700343396H1 | |
| Percent of clones expressed: 0.24 | |
| Cluster number: 115 | |
| Cluster clones: 700342616H1 700342915H1 700342708H1 | |
| Percent of clones expressed: 0.24 | |
| Cluster number: 249 | |
| Cluster clones: 700344155H1 700343631H1 700346544H1 | |
| Percent of clones expressed: 0.24 | |
| Homologous match to | |
| g498906 RPL27-5; ribosomal protein L27 homolog | |
| Cluster number: 85 | |
| Cluster clones: 700345113H1 700347107H1 700342480H1 | |
| Percent of clones expressed: 0.24 | |
| Homologous match to | |
| g391875 40S subunit ribosomal protein | |
| Cluster number: 282 | |
| Cluster clones: 700344017H1 700344618H1 700380432H1 | |
| Percent of clones expressed: 0.24 | |
| Weak match to | |
| g2264305 Arabidopsis thaliana genomic DNA, chromosome 5, P1 clone: MBK23, | |
| complete sequence. | |
| Cluster number: 348 | |
| Cluster clones: 700346344H1 700345483H1 700380044H1 | |
| Percent of clones expressed: 0.24 | |
| Homologous match to | |
| g2058311 CCR; cinnamoyl-CoA reductase; EC 1.2.1.44 | |
| Cluster number: 157 | |
| Cluster clones: 700342870H1 700346859H1 700344745H1 | |
| Percent of clones expressed: 0.24 | |
| Homologous match to | |
| g895891 RPS5; ribosomal protein S5 | |
| Cluster number: 109 | |
| Cluster clones: 700345760H1 70034257731 700345661H1 | |
| Percent of clones expressed: 0.24 | |
| Homologous match to | |
| g1405747 PR264/SC35 | |
| Cluster number: 75 | |
| Cluster clones: 700342413H1 700345249H1 700343816H1 | |
| Percent of clones expressed: 0.24 | |
| Cluster number: 80 | |
| Cluster clones: 700344040H1 700342584H1 700342440H1 | |
| Percent of clones expressed: 0.24 | |
| Homologous match to | |
| g2632156 Aspergillus niger mRNA for ribosomal protein L15. | |
| Cluster number: 128 | |
| Cluster clones: 700342671H1 700345488H1 700342885H1 | |
| Percent of clones expressed: 0.24 | |
| Homologous match to | |
| g2351063 Arabidopsis thaliana genomic DNA, chromosome 5, P1 clone: MCL19. | |
| Cluster number: 374 | |
| Cluster clones: 700346568H1 700346845H1 700346032H1 | |
| Percent of clones expressed: 0.24 | |
| Homologous match to | |
| g2564003 proteasome p45/SUG | |
| Cluster number: 91 | |
| Cluster clones: 700344541H1 700345258H1 700342510H1 | |
| Percent of clones expressed: 0.24 | |
| Cluster number: 41 | |
| Cluster clones: 700342243H1 700347105H1 700344953H1 | |
| Percent of clones expressed: 0.24 | |
| Homologous match to | |
| g2511572 prcga; multicatalytic endopeptidase; EC 3.4.99.46 | |
| Cluster number: 184 | |
| Cluster clones: 700346860H1 700343310H1 700343060H1 | |
| Percent of clones expressed: 0.24 | |
| Cluster number: 22 | |
| Cluster clones: | |
| 700281915H1 700342481H1 700346092H1 | |
| Percent of clones expressed: 0.24 | |
| Homologous match to | |
| g290275 M(3)95A; multifunctional activity and location; ribosomal protein | |
| S3/AP endonuclease DNA repair protein | |
| Cluster number: 146 | |
| Cluster clones: 700379637H1 700380108H1 700342784H1 | |
| Percent of clones expressed: 0.24 | |
| Homologous match to | |
| g2673867 Antirrhinum majus mRNA for fimbriata-associated protein 1, | |
| partial. | |
| Cluster number: 352 | |
| Cluster clones: 700345529H1 700346036H1 | |
| Percent of clones expressed: 0.16 | |
| Cluster number: 224 | |
| Cluster clones: 700343420H1 700345705H1 | |
| Percent of clones expressed: 0.16 | |
| Cluster number: 369 | |
| Cluster clones: 700346049H1 700345906H1 | |
| Percent of clones expressed: 0.16 | |
| Weak match to | |
| g2792216 b8; NBS-LRR type resistance protein | |
| Cluster number: 299 | |
| Cluster clones: 700345289H1 700344218H1 | |
| Percent of clones expressed: 0.16 | |
| Cluster number: 403 | |
| Cluster clones: 700347367H1 700380530H1 | |
| Percent of clones expressed: 0.16 | |
| Cluster number: 193 | |
| Cluster clones: 700343124H1 700345807H1 | |
| Percent of clones expressed: 0.16 | |
| Weak match to | |
| g2230869 ferritin L-chain | |
| Cluster number: 333 | |
| Cluster clones: 700345048H1 700346885H1 | |
| Percent of clones expressed: 0.16 | |
| Homologous match to | |
| g2564233 Ci-zeta; proteasome Z subunit | |
| Cluster number: 391 | |
| Cluster clones: 700346496H1 700347296H1 | |
| Percent of clones expressed: 0.16 | |
| Cluster number: 131 | |
| Cluster clones: 700380055H1 700342695H1 | |
| Percent of clones expressed: 0.16 | |
| Homologous match to | |
| g1167953 putative 32.6 kDa jasmonate-induced protein | |
| Cluster number: 201 | |
| Cluster clones: 700345404H1 700343218H1 | |
| Percent of clones expressed: 0.16 | |
| Cluster number: 116 | |
| Cluster clones: 700342620H1 700346951H1 | |
| Percent of clones expressed: 0.16 | |
| Cluster number: 372 | |
| Cluster clones: 700345964H1 700346062H1 | |
| Percent of clones expressed: 0.16 | |
| Homologous match to | |
| g2564046 Arabidopsis thaliana genomic DNA, chromosome 5, P1 clone: MCI19, | |
| complete sequence. | |
| Cluster number: 314 | |
| Cluster clones: 700344747H1 700344740H1 | |
| Percent of clones expressed: 0.16 | |
| Homologous match to | |
| g303855 ribosomal protein L7A | |
| Cluster number: 244 | |
| Cluster clones: 700345737H1 700343607H1 | |
| Percent of clones expressed: 0.16 | |
| Weak match to | |
| g1001957 ZNF127 | |
| Cluster number: 389 | |
| Cluster clones: 700346426H1 700346581H1 | |
| Percent of clones expressed: 0.16 | |
| Weak match to | |
| g1134968 protein disulphide isomerase PDI; EC 5.3.4.1 | |
| Cluster number: 283 | |
| Cluster clones: 700344122H1 700344022H1 | |
| Percent of clones expressed: 0.16 | |
| Cluster number: 167 | |
| Cluster clones: 700343909H1 700342936H1 | |
| Percent of clones expressed: 0.16 | |
| Homologous match to | |
| g1125690 S. tuberosum mRNA for DnaJ protein. | |
| Cluster number: 392 | |
| Cluster clones: 700346653H1 700347087H1 | |
| Percent of clones expressed: 0.16 | |
| Homologous match to | |
| g2656031 Arabidopsis thaliana genomic DNA, chromosome 5, P1 clone: MXC20. | |
| Cluster number: 43 | |
| Cluster clones: 700342258H1 700343458H1 | |
| Percent of clones expressed: 0.16 | |
| Homologous match to | |
| g6815 R08D7.3 | |
| Cluster number: 334 | |
| Cluster clones: 700345908H1 700345075H1 | |
| Percent of clones expressed: 0.16 | |
| Weak match to | |
| g2226414 SPBC3lF10.03; hypothetical protein | |
| Cluster number: 384 | |
| Cluster clones: 700346316H1 700347116H1 | |
| Percent of clones expressed: 0.16 | |
| Cluster number: 315 | |
| Cluster clones: 700344750H1 700344754H1 | |
| Percent of clones expressed: 0.16 | |
| Cluster number: 326 | |
| Cluster clones: 700344924H1 700345329H1 | |
| Percent of clones expressed: 0.16 | |
| Cluster number: 373 | |
| Cluster clones: 700346023H1 700346152H1 | |
| Percent of clones expressed: 0.16 | |
| Cluster number: 245 | |
| Cluster clones: 700344628H1 700343609H1 | |
| Percent of clones expressed: 0.16 | |
| Cluster number: 217 | |
| Cluster clones: 700345577H1 700343377H1 | |
| Percent of clones expressed: 0.16 | |
| Weak match to | |
| g407800 G. hirsutum mRNA for ribosomal protein 41, large subunit (RL41). | |
| Cluster number: 34 | |
| Cluster clones: 700343411H1 700342211H1 | |
| Percent of clones expressed: 0.16 | |
| Homologous match to | |
| g2102693 Frk2; fructokinase; EC 2.7.1.4 | |
| Cluster number: 12 | |
| Cluster clones: 700342744H1 | |
| 700281839H2 | |
| Percent of clones expressed: 0.16 | |
| Homologous match to | |
| g2791947 Lupinus luteus mRNA for ribosomal protein L13a. | |
| Cluster number: 200 | |
| Cluster clones: 700380616H1 700343216H1 | |
| Percent of clones expressed: 0.16 | |
| Cluster number: 130 | |
| Cluster clones: 700344785H1 700342680H1 | |
| Percent of clones expressed: 0.16 | |
| Cluster number: 306 | |
| Cluster clones: 700347360H1 700344337H1 | |
| Percent of clones expressed: 0.16 | |
| Homologous match to | |
| g2343181 MDL3; catalyzes dissociation of (R)-mandelonltrile to hydrogen | |
| cyanide and benzaldehyde; (R)-(+)-mandelonitrlle lyase isoform MDL3 precursor; | |
| EC 4.1.2.10 | |
| Cluster number: 364 | |
| Cluster clones: 700346658H1 700345859H1 | |
| Percent of clones expressed: 0.16 | |
| Cluster number: 207 | |
| Cluster clones: 700346745H1 700343266H1 | |
| Percent of clones expressed: 0.16 | |
| Weak match to | |
| g577612 CRT1; calcium sequestration; calcium-binding protein; calreticulin | |
| Cluster number: 265 | |
| Cluster clones: 700343792H1 700345881H1 | |
| Percent of clones expressed: 0.16 | |
| Homologous match to | |
| g2827618 Arabidopsis thaliana DNA chromosome 4, BAC clone F10N7 (ESSAII | |
| project). | |
| Cluster number: 58 | |
| Cluster clones: 700344016H1 700342338H1 | |
| Percent of clones expressed: 0.16 | |
| Cluster number: 405 | |
| Cluster clones: 700380270H1 700379610H1 | |
| Percent of clones expressed: 0.16 | |
| Cluster number: 108 | |
| Cluster clones: 700342570H1 700380047H1 | |
| Percent of clones expressed: 0.16 | |
| Homologous match to | |
| g791101 Yeast (Saccharomyces cerevlsiae) PMS1, TPM1, MKS1, YMK1, MSK1, | |
| ODP2, YL9A & FKH2 genes. | |
| Cluster number: 335 | |
| Cluster clones: 700346935H1 700345079H1 | |
| Percent of clones expressed: 0.16 | |
| Weak match to | |
| g2618602 Arabidopsis thaliana genomic DNA, chromosome 5, P1 clone: MSJ1, | |
| complete sequence. | |
| Cluster number: 383 | |
| Cluster clones: 700347092H1 700346264H1 | |
| Percent of clones expressed: 0.16 | |
| Cluster number: 393 | |
| Cluster clones: 700380571H1 700346656H1 | |
| Percent of clones expressed: 0.16 | |
| Cluster number: 72 | |
| Cluster clones: 700343601H1 700342401H1 | |
| Percent of clones expressed: 0.16 | |
| Homologous match to | |
| g2190545 F5I14.8 | |
| Cluster number: 195 | |
| Cluster clones: 700344031H1 700343151H1 | |
| Percent of clones expressed: 0.16 | |
| Homologous match to | |
| g1502354 Yeast (Saccharomyces cerevisiae) genomic sequence from chromosome | |
| VII. | |
| Cluster number: 328 | |
| Cluster clones: 700345293H1 700345004H1 | |
| Percent of clones expressed: 0.16 | |
| Cluster number: 316 | |
| Cluster clones: 700344763H1 700346304H1 | |
| Percent of clones expressed: 0.16 | |
| Homologous match to | |
| g1223910 chaperonin-60 alpha subunit | |
| Cluster number: 176 | |
| Cluster clones: 700342986H1 700380488H1 | |
| Percent of clones expressed: 0.16 | |
| Cluster number: 386 | |
| Cluster clones: 700346539H1 700346336H1 | |
| Percent of clones expressed: 0.16 | |
| Homologous match to | |
| g2828188 Arabidopsis thaliana genomic DNA, chromosome 5, TAC clone: K313, | |
| complete sequence. | |
| Cluster number: 241 | |
| Cluster clones: 700343559H1 700347255H1 | |
| Percent of clones expressed: 0.16 | |
| Homologous match to | |
| g440952 cytochrome c1 [Solanum tuberosum = potatoes, Desiree, monohaploid | |
| line AM 80/5793, Genomic, 6007 nt] | |
| Cluster number: 210 | |
| Cluster clones: 700344191H1 700343291H1 | |
| Percent of clones expressed: 0.16 | |
| Weak match to | |
| g872116 sti (stress inducible protein) | |
| Cluster number: 140 | |
| Cluster clones: 700342747H1 700343755H1 | |
| Percent of clones expressed: 0.16 | |
| Cluster number: 24 | |
| Cluster clones: 700343834H1 | |
| 700281924H2 | |
| Percent of clones expressed: 0.16 | |
| Homologous match to | |
| g1100222 Pinus sylvestris chloroplast NAD+-dependent | |
| glyceraldehyde-3-phosphate dehydrogenase mRNA, complete cds. | |
| Cluster number: 31 | |
| Cluster clones: | |
| 700281973H1 | |
| 700281986H2 | |
| Percent of clones expressed: 0.16 | |
| Weak match to | |
| g1946354 Arabidopsis thaliana chromosome II BAC T06B20 genomic sequence, | |
| complete sequence. | |
| Cluster number: 350 | |
| Cluster clones: 700345516H1 700346894H1 | |
| Percent of clones expressed: 0.16 | |
| Weak match to | |
| g2749918 Arabidopsis thaliana chromosome I BAC F3I6 genomic sequence, | |
| complete sequence. | |
| Cluster number: 266 | |
| Cluster clones: 700346704H1 700343793H1 | |
| Percent of clones expressed: 0.16 | |
| Weak match to | |
| g1903420 p71 | |
| Cluster number: 406 | |
| Cluster clones: 700379874H1 700379739H1 | |
| Percent of clones expressed: 0.16 | |
| Homologous match to | |
| g556217 spliceosomal protein | |
| Cluster number: 196 | |
| Cluster clones: 700347236H1 700343154H1 | |
| Percent of clones expressed: 0.16 | |
| Weak match to | |
| g475212 N. tabacum eIF-4A10 mRNA. | |
| Cluster number: 190 | |
| Cluster clones: 700343880H1 700343108H1 | |
| Percent of clones expressed: 0.16 | |
| Homologous match to | |
| g218099 Rice mRNA for ribosomal protein S12 (320 gene), partial sequence. | |
| Cluster number: 138 | |
| Cluster clones: 700342736H1 700345985H1 | |
| Percent of clones expressed: 0.16 | |
| Homologous match to | |
| g2564336 Brassica campestris mRNA for Tat binding protein 1, complete cds. | |
| Cluster number: 336 | |
| Cluster clones: 700345291H1 700345094H1 | |
| Percent of clones expressed: 0.16 | |
| Cluster number: 230 | |
| Cluster clones: 700343475H1 700380514H1 | |
| Percent of clones expressed: 0.16 | |
| Cluster number: 132 | |
| Cluster clones: 700347059H1 700342701H1 | |
| Percent of clones expressed: 0.16 | |
| Homologous match to | |
| g2464876 patatin homolog | |
| Cluster number: 55 | |
| Cluster clones: 700343509H1 700342309H1 | |
| Percent of clones expressed: 0.16 | |
| Weak match to | |
| g1151003 hypothetical protein UNF346 | |
| Cluster number: 367 | |
| Cluster clones: 700346091H1 700345902H1 | |
| Percent of clones expressed: 0.16 | |
| Homologous match to | |
| g1235572 P. curvlcoila EF1-alpha gene. | |
| Cluster number: 297 | |
| Cluster clones: 700344186H1 700344295H1 | |
| Percent of clones expressed: 0.16 | |
| Cluster number: 375 | |
| Cluster clones: 700380786H1 700346050H1 | |
| Percent of clones expressed: 0.16 | |
| Weak match to | |
| g310934 Nicotiana tabacum ribosomal protein L25, complete cds. | |
| Cluster number: 317 | |
| Cluster clones: 700379650H1 700344782H1 | |
| Percent of clones expressed: 0.16 | |
| Cluster number: 239 | |
| Cluster clones: 700343552H1 700345739H1 | |
| Percent of clones expressed: 0.16 | |
| Cluster number: 211 | |
| Cluster clones: 700343985H1 700343292H1 | |
| Percent of clones expressed: 0.16 | |
| Cluster number: 40 | |
| Cluster clones: 700343431H1 700342231H1 | |
| Percent of clones expressed: 0.16 | |
| Cluster number: 191 | |
| Cluster clones: 700343608H1 700343110H1 | |
| Percent of clones expressed: 0.16 | |
| Homologous match to | |
| g2832667 Arabidopsis thaliana DNA chromosome 4, BAC clone T10I14 (ESSAII | |
| project). | |
| Cluster number: 331 | |
| Cluster clones: 700346887H1 700345013H1 | |
| Percent of clones expressed: 0.16 | |
| Weak match to | |
| g1143863 Oryza sativa beta-glucosidase mRNA, nuclear gene encoding | |
| chloroplast protein, complete cds. | |
| Cluster number: 122 | |
| Cluster clones: 700342655H1 700346488H1 | |
| Percent of clones expressed: 0.16 | |
| Homologous match to | |
| g2243116 | ak-lys1; aspartate kinase; EC 2.7.2.4 |
| Cluster number: 260 | |
| Cluster clones: 700345785H1 700343746H1 | |
| Percent of clones expressed: 0.16 | |
| Weak match to | |
| g1469187 The KIAA0132 gene product is related to Drosophila melanogaster | |
| ring canel protein. | |
| Cluster number: 261 | |
| Cluster clones: 700346943H1 700343757H1 | |
| Percent of clones expressed: 0.16 | |
| Cluster number: 203 | |
| Cluster clones: 700343225H1 700343325H1 | |
| Percent of clones expressed: 0.16 | |
| Cluster number: 14 | |
| Cluster clones: | |
| 700281844H2 700346077H1 | |
| Percent of clones expressed: 0.16 | |
| Homologous match to | |
| g710626 ERD15; ERDl5 protein | |
| Cluster number: 79 | |
| Cluster clones: 700342434H1 70034250H1 | |
| Percent of clones expressed: 0.16 | |
| Homologous match to | |
| g2130984 B. napus mRNA for ribosomal protein S15a (pA8l3). | |
| Cluster number: 401 | |
| Cluster clones: 700347395H1 700347064H1 | |
| Percent of clones expressed: 0.16 | |
| Cluster number: 93 | |
| Cluster clones: 700343715H1 700342515H1 | |
| Percent of clones expressed: 0.16 | |
| Cluster number: 407 | |
| Cluster clones: 700380278H1 700379763H1 | |
| Percent of clones expressed: 0.16 | |
| Homologous match to | |
| g1911165 S. tuberosum mRNA for soluble-starch-synthase. | |
| Cluster number: 139 | |
| Cluster clones: 700342745H1 700344965H1 | |
| Percent of clones expressed: 0.16 | |
| Homologous match to | |
| g430992 protein kinase | |
| Cluster number: 278 | |
| Cluster clones: 700346026H1 700343965H1 | |
| Percent of clones expressed: 0.16 | |
| Cluster number: 337 | |
| Cluster clones: 700345136H1 700345235H1 | |
| Percent of clones expressed: 0.16 | |
| Cluster number: 267 | |
| Cluster clones: 700343807H1 700346108H1 | |
| Percent of clones expressed: 0.16 | |
| Homologous match to | |
| g1341398 A. thaliana mRNA for AtTFIIB2 protein. | |
| Cluster number: 103 | |
| Cluster clones: 700346351H1 700342543H1 | |
| Percent of clones expressed: 0.16 | |
| Weak match to | |
| g895693 ribosomal protein S15a | |
| Cluster number: 301 | |
| Cluster clones: 700344265H1 700347232H1 | |
| Percent of clones expressed: 0.16 | |
| Cluster number: 238 | |
| Cluster clones: 700343540H1 700343790H1 | |
| Percent of clones expressed: 0.16 | |
| Cluster number: 231 | |
| Cluster clones: 700343477H1 700345376H1 | |
| Percent of clones expressed: 0.16 | |
| Homologous match to | |
| g2352928 Petroselinum crispum plastidic chorismate mutase 1 (pCM1) mRNA, | |
| nuclear gene encoding plastid protein, partial cds. | |
| Cluster number: 168 | |
| Cluster clones: 700344203H1 700342945H1 | |
| Percent of clones expressed: 0.16 | |
| Homologous match to | |
| g602302 DIM; cell elongation; diminuto | |
| Cluster number: 318 | |
| Cluster clones: 700380480H1 700344836H1 | |
| Percent of clones expressed: 0.16 | |
| Homologous match to | |
| g2689242 A. thaliana mRNA for ubiquitin conjugating enzyme UBC2. | |
| Cluster number: 308 | |
| Cluster clones: 700344425H1 700347072H1 | |
| Percent of clones expressed: 0.16 | |
| Weak match to | |
| g1381148 Hs-CUL-4A | |
| Cluster number: 376 | |
| Cluster clones: 700346060H1 700346167H1 | |
| Percent of clones expressed: 0.16 | |
| Cluster number: 142 | |
| Cluster clones: 700342755H1 700347050H1 | |
| Percent of clones expressed: 0.16 | |
| Weak match to | |
| g2244788 Arabidopsis thaliana DNA chromosome 4, ESSA I contig fragment No. | |
| 1. | |
| Cluster number: 410 | |
| Cluster clones: 700380452H1 700380146H1 | |
| Percent of clones expressed: 0.16 | |
| Weak match to | |
| g786228 tRNA-Gly | |
| Cluster number: 38 | |
| Cluster clones: 700342228H1 700343428H1 | |
| Percent of clones expressed: 0.16 | |
| Cluster number: 270 | |
| Cluster clones: 700343832H1 700347126H1 | |
| Percent of clones expressed: 0.16 | |
| Cluster number: 212 | |
| Cluster clones: 700343333H1 700345469H1 | |
| Percent of clones expressed: 0.16 | |
| Weak match to | |
| g2828287 T18B16.90; putative protein | |
| Cluster number: 312 | |
| Cluster clones: 700344691H1 700345118H1 | |
| Percent of clones expressed: 0.16 | |
| Homologous match to | |
| g624956 C. reinhardtii mRNA for ribosomal protein S18. | |
| Cluster number: 287 | |
| Cluster clones: 700344086H1 700346687H1 | |
| Percent of clones expressed: 0.16 | |
| Homologous match to | |
| g1843628 bsh; SNF5 homolog BSH | |
| Cluster number: 357 | |
| Cluster clones: 700345571H1 700345671H1 | |
| Percent of clones expressed: 0.16 | |
| Weak match to | |
| g2583135 F4L23.30 | |
| Cluster number: 242 | |
| Cluster clones: 700346525H1 700343572H1 | |
| Percent of clones expressed: 0.16 | |
| Cluster number: 296 | |
| Cluster clones: 700344174H1 700346848H1 | |
| Percent of clones expressed: 0.16 | |
| Cluster number: 181 | |
| Cluster clones: 700343049H1 700343995H1 | |
| Percent of clones expressed: 0.16 | |
| Homologous match to | |
| g633606 P. sativum mRNA for chloroplastic outer envelope membrane protein | |
| (OEP75). | |
| Cluster number: 95 | |
| Cluster clones: 700342523H1 700343723H1 | |
| Percent of clones expressed: 0.16 | |
| Cluster number: 396 | |
| Cluster clones: 700346838H1 700347011H1 | |
| Percent of clones expressed: 0.16 | |
| Homologous match to | |
| g1946689 Arabidopsis thaliana glutathione peroxidase GPx mRNA, complete | |
| cds. | |
| Cluster number: 268 | |
| Cluster clones: 700343811H1 700380748H1 | |
| Percent of clones expressed: 0.16 | |
| Homologous match to | |
| g2459531 Mf-FatB1; acyl-ACP thioesterase | |
| Cluster number: 172 | |
| Cluster clones: 700342959H1 700346389H1 | |
| Percent of clones expressed: 0.16 | |
| Weak match to | |
| g1749440 similar to Saccharomyces cerevisiae unknown, EMBL Accession Number | |
| Z47815 | |
| Cluster number: 338 | |
| Cluster clones: 700345221H1 700345489H1 | |
| Percent of clones expressed: 0.16 | |
| Homologous match to | |
| g434905 A. thaliana (Columbia) gene for S18 ribosomal protein (1471bp). | |
| Cluster number: 83 | |
| Cluster clones: 700342456H1 700345264H1 | |
| Percent of clones expressed: 0.16 | |
| Homologous match to | |
| g2258073 Hordeum vulgare var. distichum soluble inorganic pyrophosphatase | |
| (Ipp) mRNA, complete cds. | |
| Cluster number: 290 | |
| Cluster clones: 700346847H1 700344120H1 | |
| Percent of clones expressed: 0.16 | |
| Homologous match to | |
| g1914684 D. carota mRNA for RAD23 protein, isoform II. | |
| Cluster number: 104 | |
| Cluster clones: 700346210H1 700342551H1 | |
| Percent of clones expressed: 0.16 | |
| Weak match to | |
| g1783230 | aminopeptidase |
| Cluster number: 370 | |
| Cluster clones: 700347308H1 700345910H1 | |
| Percent of clones expressed: 0.16 | |
| Weak match to | |
| g2245126 Arabidopsis thaliana DNA chromosome 4, ESSA I contig fragment No. | |
| 9. | |
| Cluster number: 329 | |
| Cluster clones: 700345008H1 700346815H1 | |
| Percent of clones expressed: 0.16 | |
| Homologous match to | |
| g2262141 T10P11.8; hypothetical protein | |
| Cluster number: 259 | |
| Cluster clones: 700345403H1 700343742H1 | |
| Percent of clones expressed: 0.16 | |
| Cluster number: 90 | |
| Cluster clones: 700343704H1 700342504H1 | |
| Percent of clones expressed: 0.16 | |
| Homologous match to | |
| g2832679 T10I14.120; putative protein | |
| Cluster number: 319 | |
| Cluster clones: 700347049H1 700344849H1 | |
| Percent of clones expressed: 0.16 | |
| Cluster number: 189 | |
| Cluster clones: 70037986251 700343103H1 | |
| Percent of clones expressed: 0.16 | |
| Cluster number: 179 | |
| Cluster clones: 700345023H1 700343021H1 | |
| Percent of clones expressed: 0.16 | |
| Homologous match to | |
| g927567 actin | |
| Cluster number: 411 | |
| Cluster clones: 700380682H1 700380589H1 | |
| Percent of clones expressed: 0.16 | |
| Cluster number: 213 | |
| Cluster clones: 700343342H1 700344920H1 | |
| Percent of clones expressed: 0.16 | |
| Cluster number: 153 | |
| Cluster clones: 700346288H1 700342832H1 | |
| Percent of clones expressed: 0.16 | |
| Cluster number: 5 | |
| Cluster clones: | |
| 700281810H2 | |
| 700281945H2 | |
| Percent of clones expressed: 0.16 | |
| Homologous match to | |
| g2529427 rpc10+; Rpc10; EC 2.7.7.6 | |
| Cluster number: 47 | |
| Cluster clones: 700342846H1 700342278H1 | |
| Percent of clones expressed: 0.16 | |
| Weak match to | |
| g2072023 symbiosis-related protein | |
| Cluster number: 288 | |
| Cluster clones: 700344091H1 700346623H1 | |
| Percent of clones expressed: 0.16 | |
| Homologous match to | |
| g1132482 Rice mRNA for ADP-ribosylation factor, complete cds. | |
| Cluster number: 322 | |
| Cluster clones: 700344878H1 700345384H1 | |
| Percent of clones expressed: 0.16 | |
| Cluster number: 124 | |
| Cluster clones: 700342666H1 700342682H1 | |
| Percent of clones expressed: 0.16 | |
| Cluster number: 252 | |
| Cluster clones: 700343672H1 700343822H1 | |
| Percent of clones expressed: 0.16 | |
| Homologous match to | |
| g2832242 Zea mays 22-kDa alpha zein gene cluster, complete sequence. | |
| Cluster number: 409 | |
| Cluster clones: 700380263H1 700380096H1 | |
| Percent of clones expressed: 0.16 | |
| Cluster number: 339 | |
| Cluster clones: 700345325H1 700345225H1 | |
| Percent of clones expressed: 0.16 | |
| Homologous match to | |
| g683502 protein phosphatase 2A 65 kDa regulatory subunit | |
| Cluster number: 291 | |
| Cluster clones: 700346008H1 700344124H1 | |
| Percent of clones expressed: 0.16 | |
| Cluster number: 187 | |
| Cluster clones: 700343087H1 700345330H1 | |
| Percent of clones expressed: 0.16 | |
| Cluster number: 233 | |
| Cluster clones: 700346413H1 700343484H1 | |
| Percent of clones expressed: 0.16 | |
| Homologous match to | |
| g836939 Arabidopsis thaliana calcium-dependent protein kinase (CDPK6) | |
| mRNA, complete cds. | |
| Cluster number: 298 | |
| Cluster clones: 700380408H1 700344217H1 | |
| Percent of clones expressed: 0.16 | |
| Homologous match to | |
| g514394 Catharanthus roseus cyco7 gene, exon1-7, complete cds. | |
| Cluster number: 73 | |
| Cluster clones: 700342403H1 700343603H1 | |
| Percent of clones expressed: 0.16 | |
| Weak match to | |
| g2635371 similar to hypothetical proteins | |
| Cluster number: 59 | |
| Cluster clones: 700342463H1 700342340H1 | |
| Percent of clones expressed: 0.16 | |
| Cluster number: 390 | |
| Cluster clones: 700346475H1 700347222H1 | |
| Percent of clones expressed: 0.16 | |
| Weak match to | |
| g170107 CPN10; chaperonin 10 | |
| Cluster number: 262 | |
| Cluster clones: 700347094H1 700343765H1 | |
| Percent of clones expressed: 0.16 | |
| Homologous match to | |
| g2342735 T14G11.28 | |
| Cluster number: 378 | |
| Cluster clones: 700347212H1 700346117H1 | |
| Percent of clones expressed: 0.16 | |
| Homologous match to | |
| g2814799 ZK858.6 | |
| Cluster number: 342 | |
| Cluster clones: 700379013H1 700345369H1 | |
| Percent of clones expressed: 0.16 | |
| Homologous match to | |
| g2244991 Arabidopsis thaliana DNA chromosome 4, ESSA I contig fragment No. | |
| 6. | |
| Cluster number: 359 | |
| Cluster clones: 700345716H1 700345686H1 | |
| Percent of clones expressed: 0.16 | |
| Cluster number: 289 | |
| Cluster clones: 700344096H1 700344196H1 | |
| Percent of clones expressed: 0.16 | |
| Cluster number: 381 | |
| Cluster clones: 700347093H1 700346218H1 | |
| Percent of clones expressed: 0.16 | |
| Cluster number: 125 | |
| Cluster clones: 700343703H1 700342667H1 | |
| Percent of clones expressed: 0.16 | |
| Cluster number: 402 | |
| Cluster clones: 700347176H1 700379975H1 | |
| Percent of clones expressed: 0.16 | |
| Cluster number: 257 | |
| Cluster clones: 700346386H1 700343711H1 | |
| Percent of clones expressed: 0.16 | |
| Homologous match to | |
| g1008175 ORF YJL045w | |
| Cluster number: 97 | |
| Cluster clones: 700342528H1 700346629H1 | |
| Percent of clones expressed: 0.16 | |
| Homologous match to | |
| g218180 Rice mRNA for oryzain alpha (EC 3.4.22). | |
| Cluster number: 292 | |
| Cluster clones: 700344130H1 700345441H1 | |
| Percent of clones expressed: 0.16 | |
| Homologous match to | |
| g457708 S. oleracea mRNA for protein kinase. | |
| Cluster number: 106 | |
| Cluster clones: 700342560H1 700380575H1 | |
| Percent of clones expressed: 0.16 | |
| Weak match to | |
| g2558659 No definition line found | |
| Cluster number: 234 | |
| Cluster clones: 700346918H1 700343487H1 | |
| Percent of clones expressed: 0.16 | |
| Cluster number: 279 | |
| Cluster clones: 700379925H1 700343991H1 | |
| Percent of clones expressed: 0.16 | |
| Weak match to | |
| g2264302 Arabidopsis thaliana genomic DNA, chromosome 5, P1 clone: MAC12, | |
| complete sequence. | |
| Cluster number: 349 | |
| Cluster clones: 700345706H1 700345506H1 | |
| Percent of clones expressed: 0.16 | |
| Homologous match to | |
| g1375074 Oryza sativa mRNA for glyoxysomal malate dehydrogenase, complete | |
| cds. | |
| Cluster number: 379 | |
| Cluster clones: 700380584H1 700346146H1 | |
| Percent of clones expressed: 0.16 | |
| Weak match to | |
| g2394431 F23F1.5 | |
| Cluster number: 343 | |
| Cluster clones: 700380683H1 700345405H1 | |
| Percent of clones expressed: 0.16 | |
| Weak match to | |
| g525196 epsilon-COP; ldlFp | |
| Cluster number: 273 | |
| Cluster clones: 700343841H1 700344655H1 | |
| Percent of clones expressed: 0.16 | |
| Weak match to | |
| g2394230 Arabidopsis thaliana WD-40 repeat protein (MSI2) mRNA, complete | |
| cds. | |
| Cluster number: 49 | |
| Cluster clones: 700342288H1 700347334H1 | |
| Percent of clones expressed: 0.16 | |
| Homologous match to | |
| g2463332 Oryza sativa mRNA for M79 protein. | |
| Cluster number: 173 | |
| Cluster clones: 700346256H1 700342961H1 | |
| Percent of clones expressed: 0.16 | |
| Homologous match to | |
| g54006 ribosomal protein S12 (AA 1 - 132) | |
| Cluster number: 56 | |
| Cluster clones: 700344212H1 700342313H1 | |
| Percent of clones expressed: 0.16 | |
| Homologous match to | |
| g2651294 Arabidopsis thaliana chromosome II BAC T2P4 genomic sequence, | |
| complete sequence. | |
| Cluster number: 63 | |
| Cluster clones: 700345747H1 700342363H1 | |
| Percent of clones expressed: 0.16 | |
| Cluster number: 112 | |
| Cluster clones: 700343789H1 700342589H1 | |
| Percent of clones expressed: 0.16 | |
| Cluster number: 240 | |
| Cluster clones: 700346939H1 700343558H1 | |
| Percent of clones expressed: 0.16 | |
| Homologous match to | |
| g499111 Flaveria bidentis clone pNDK-b nucleoside diphosphate kinase mRNA, | |
| complete cds. | |
| Cluster number: 399 | |
| Cluster clones: 700346996H1 700379719H1 | |
| Percent of clones expressed: 0.16 | |
| Cluster number: 305 | |
| Cluster clones: 700345020H1 700344334H1 | |
| Percent of clones expressed: 0.16 | |
| Cluster number: 107 | |
| Cluster clones: 700342567H1 700345336H1 | |
| Percent of clones expressed: 0.16 | |
| Homologous match to | |
| g2281103 P18019.22; Glucan endo-1,3-beta glucosidase isolog | |
| Cluster number: 170 | |
| Cluster clones: 700347040H1 700342952H1 | |
| Percent of clones expressed: 0.16 | |
| Weak match to | |
| g2317855 MMS2; provides resistance to DNA damaging agents; Mms2p | |
| Cluster number: 388 | |
| Cluster clones: 700346392H1 700347238H1 | |
| Percent of clones expressed: 0.16 | |
| Homologous match to | |
| g553063 GST2; glutathione s-transferase | |
| Cluster number: 344 | |
| Cluster clones: 700345873H1 700345412H1 | |
| Percent of clones expressed: 0.16 | |
| Cluster number: 92 | |
| Cluster clones: 700343762H1 700342512H1 | |
| Percent of clones expressed: 0.16 | |
| Homologous match to | |
| g556018 Barley glutamate 1-semialdehyde aminotransferase (GSA) mRNA, | |
| complete cds. | |
| Cluster number: 78 | |
| Cluster clones: 700343632H1 700342432H1 | |
| Percent of clones expressed: 0.16 | |
| Cluster number: 274 | |
| Cluster clones: 700344454H1 700343842H1 | |
| Percent of clones expressed: 0.16 | |
| Weak match to | |
| g189306 nucleolin | |
| Cluster number: 60 | |
| Cluster clones: 700342354H1 700344629H1 | |
| Percent of clones expressed: 0.16 | |
| Homologous match to | |
| g1061037 A. thaliana mRNA for sterol-C5-desaturase. | |
| Cluster number: 325 | |
| Cluster clones: 700344911H1 700346157H1 | |
| Percent of clones expressed: 0.16 | |
| Cluster number: 276 | |
| Cluster clones: 700343875H1 700344928H1 | |
| Percent of clones expressed: 0.16 | |
| Cluster number: 255 | |
| Cluster clones: 700344952H1 700343701H1 | |
| Percent of clones expressed: 0.16 | |
| Homologous match to | |
| g313266 T. aestivum gene for phosphoglycerate kinase. | |
| Cluster number: 154 | |
| Cluster clones: 700344649H1 700342835H1 | |
| Percent of clones expressed: 0.16 | |
| Cluster number: 127 | |
| Cluster clones: 700342670H1 700344828H1 | |
| Percent of clones expressed: 0.16 | |
| Cluster number: 4 | |
| Cluster clones: 700355947H1 700353526H1 700332063H1 700352605H1 700353271H1 | |
| 700382691H1 700353955H1 700382739H1 700382890H1 700355520H1 700355738H1 | |
| 700382702H1 700353625H1 700356140H1 | |
| 700282414H2 700355431H1 700355029H1 700355925H1 700356466H1 700355428H1 | |
| 700382605H1 700354129H1 700355088H1 | |
| Percent of clones expressed: 2.45 | |
| Weak match to | |
| g415896 | PHL P11, pollen allergen |

Cluster number: 25
Cluster clones: 700382183H1 700356708H1 700357108H1 700357122H1 700356474H1
700353005H1 700355779H1 700353128H1
700282575H2 700352675H1 700352843H1
Percent of clones expressed: 1.17
Weak match to
g2829898 T26J12.9
Cluster number: 66
Cluster clones: 700355738H1 700353019H1 700352940H1 700354759H1 700382843H1
700356461H1 700382351H1 700354906H1 700382907H1 700353011H1
Percent of clones expressed: 1.06
Homologous match to
g509769 seed-specific low molecular weight sulfur-rich protein
Cluster number: 42
Cluster clones: 700352709H1 700352735H1 700355462H1 700355558H1 700353828H1
700356627H1 700382275H1 700356219H1 700383158H1
Percent of clones expressed: 0.96
Cluster number: 125
Cluster clones: 700382282H1 700356290H1 700356027H1 700354886H1 700382607H1
700357164H1 700382281H1 700353883H1
Percent of clones expressed: 0.35
Homologous match to
g533692 protease inhibitor
Cluster number: 141
Cluster clones: 700355547H1 700355026H1 700354647H1 700355009H1 700354337H1
700356158H1 700354908H1
Percent of clones expressed: 0.74
Weak match to
g2707295 root allergen protein
Cluster number: 30
Cluster clones: 700356129H1 700382106H1 700352616H1 700354518H1 700356066H1
700356023H1 700382827H1
Percent of clones expressed: 0.74
Cluster number: 105
Cluster clones: 700353446H1 700355016H1 700353409H1 700383149H1 700382979H1
700355249H1
Percent of clones expressed: 0.64
Cluster number: 75
Cluster clones: 700353007H1 700383013H1 700383021H1 700354239H1 700382708H1
700356020H1
Percent of clones expressed: 0.64
Cluster number: 71
Cluster clones: 700382061H1 700356143H1 700353275H1 700353688H1 700357428H1
700352972H1
Percent of clones expressed: 0.64
Homologous match to
g255569 pollen specific protein
Cluster number: 15
Cluster clones: 700382195H1
700282503H2 700353432H1 700382995H1 700354850H1
Percent of clones expressed: 0.53
Cluster number: 126
Cluster clones: 700353887H1 700355754H1 700354961H1 700355306H1 700354422H1
Percent of clones expressed: 0.53
Homologous match to
g1173556 Oryza sativa Ory s 1 mRNA, complete cds.
Cluster number: 86
Cluster clones: 700353284H1 700382179H1 700354875H1 700382668H1 700353052H1
Percent of clones expressed: 0.53
Cluster number: 122
Cluster clones: 700382209H1 700356196H1 700353823H1 700356180H1 700355590H1
Percent of clones expressed: 0.53
Homologous match to
g2384757 Oryza sativa GDP dissociation inhibitor protein OsGDI1 (OsGDI1)
mRNA, complete cds.
Cluster number: 11
Cluster clones:
700282464H1 700354271H1 700355316H1 700383159H1
Percent of clones expressed: 0.43
Homologous match to
g22428 polygalacturonase
Cluster number: 181
Cluster clones: 700355095H1 700382374H1 700355484H1 700355710H1
Percent of clones expressed: 0.43
Homologous match to
g20163 O. sativa Rr15 mRNA for 5S ribosomal RNA.
Cluster number: 82
Cluster clones: 700353142H1 700353028H1 700382229H1 700357402H1
Percent of clones expressed: 0.43
Weak match to
g2055230 src2; SRC2; soybean cold-regulated gene
Cluster number: 33
Cluster clones: 700357489H1 700382191H1 700382543H1 700352625H1
Percent of clones expressed: 0.43
Cluster number: 202
Cluster clones: 700382975H1 700355963H1 700382392H1 700355889H1
Percent of clones expressed: 0.43
Weak match to
g2827559 T12H17.210; predicted protein
Cluster number: 2
Cluster clones:
700282406H2
700282408H2
700282409H2
700282410H2
Percent of clones expressed: 0.43
Homologous match to
g313137 Z. mays ZmPRO1 mRNA for profilin 1.
Cluster number: 100
Cluster clones: 700356964H1 700355191H1 700382231H1 700353256H1
Percent of clones expressed: 0.43
Homologous match to
g1370199 L. japonicus mRNA for small GTP-binding protein, RAC1.
Cluster number: 156
Cluster clones: 700355893H1 700355171H1 700354536H1 700356157H1
Percent of clones expressed: 0.43
Homologous match to
g2760330 F1N2l.15
Cluster number: 118
Cluster clones: 700354689H1 700382139H1 700353629H1 700354676H1
Percent of clones expressed: 0.43
Cluster number: 87
Cluster clones: 700357445H1 700353057H1 700382230H1 700382190H1
Percent of clones expressed: 0.43
Cluster number: 63
Cluster clones: 700383154H1 700352912H1 700354742H1 700354895H1
Percent of clones expressed: 0.43
Cluster number: 121
Cluster clones: 700353779H1 700355415H1 700357160H1 700355414H1
Percent of clones expressed: 0.43
Weak match to
g1835730 Oryza sativa photosystem II 10 kDa polypeptide mRNA, complete cds.
Cluster number: 13
Cluster clones: 700352678H1
700282478H2 700355878H1
Percent of clones expressed: 0.32
Homologous match to
g293901 Zea mays Zea mI gene, complete cds.
Cluster number: 6
Cluster clones:
700282435H1
700282569H2
700282571H2
Percent of clones expressed: 0.32
Cluster number: 52
Cluster clones: 700352780H1 700352862H1 700354483H1
Percent of clones expressed: 0.32
Homologous match to
g1890351 A. thaliana mRNA for transcription factor L2.
Cluster number: 45
Cluster clones: 700355053H1 700357189H1 700352737H1
Percent of clones expressed: 0.32
Cluster number: 38
Cluster clones: 700353424H1 700352654H1 700352719H1
Percent of clones expressed: 0.32
Cluster number: 78
Cluster clones: 700383062H1 700353023H1 700355781H1
Percent of clones expressed: 0.32
Weak match to
g395316 P450CR10; cytochrome P450
Cluster number: 62
Cluster clones: 700355973H1 700356535H1 700352911H1
Percent of clones expressed: 0.32
Homologous match to
g544501 SEC13; Sec13p
Cluster number: 90
Cluster clones: 700382353H1 700355513H1 700353114H1
Percent of clones expressed: 0.32
Cluster number: 116
Cluster clones: 700353610H1 700356435H1 700356494H1
Percent of clones expressed: 0.32
Homologous match to
g2852444 Salix bakko mRNA for SUI1 homolog, complete cds.
Cluster number: 124
Cluster clones: 700353853H1 700383155H1 700382218H1
Percent of clones expressed: 0.32
Cluster number: 163
Cluster clones: 700356609H1 700354660H1 700356827H1
Percent of clones expressed: 0.32
Homologous match to
g606969 Arabidopsis thaliana cytoplasmic ribosomal protein L18 mRNA,
complete cds.
Cluster number: 10
Cluster clones: 700382655H1
700282457H2 700355978H1
Percent of clones expressed: 0.32
Homologous match to
g1718236 (1,4)-beta-xylan endohydrolase, isoenzyme X-I
Cluster number: 196
Cluster clones: 700355783H1 700382507H1 700356725H1
Percent of clones expressed: 0.32
Weak match to
g2351063 Arabidopsis thaliana genomic DNA, chromosome 5, P1 clone: MCL19.
Cluster number: 77
Cluster clones: 700382817H1 700353014H1 700354142H1
Percent of clones expressed: 0.32
Weak match to
g1749610 similar to Saccharomyces cerevisiae unknown, EMBL Accession Number
Z68195
Cluster number: 214
Cluster clones: 700382245H1 700356534H1 700382140H1
Percent of clones expressed: 0.32
Cluster number: 91
Cluster clones: 700382267H1 700353130H1 700354478H1
Percent of clones expressed: 0.32
Weak match to
g2058693 poly A binding protein
Cluster number: 97
Cluster clones: 700353206H1 700382119H1 700382127H1
Percent of clones expressed: 0.32
Homologous match to
g669003 calnexin
Cluster number: 99
Cluster clones: 700356188H1 700382662H1 700353245H1
Percent of clones expressed: 0.32
Homologous match to
g255568 pollen specific protein [Zea mays = corn, mRNA, 943 nt].
Cluster number: 107
Cluster clones: 700353509H1 700355885H1 700353612H1
Percent of clones expressed: 0.32
Cluster number: 17
Cluster clones:
700282520H2
700282517H2
700282518H2
Percent of clones expressed: 0.32
Homologous match to
g2656030 Arabidopsis thaliana genomic DNA, chromosome 5, P1 clone: MUL8.
Cluster number: 20
Cluster clones:
700282549H2
700282547H2
Percent of clones expressed: 0.21
Cluster number: 109
Cluster clones: 700353532H1 700353844H1
Percent of clones expressed: 0.21
Homologous match to
g22458 Z. mays pollen specific mRNA C-terminal (clone 4H7)
Cluster number: 224
Cluster clones: 700382055H1 700382544H1
Percent of clones expressed: 0.21
Homologous match to
g2252869 ATOZI1
Cluster number: 205
Cluster clones: 700356171H1 700382075H1
Percent of clones expressed: 0.21
Cluster number: 193
Cluster clones: 700382330H1 700355577H1
Percent of clones expressed: 0.21
Cluster number: 135
Cluster clones: 700354374H1 700354295H1
Percent of clones expressed: 0.21
Homologous match to
g1480465 cyclophilin Ovcyp-2
Cluster number: 237
Cluster clones: 700382426H1 700382525H1
Percent of clones expressed: 0.21
Weak match to
g475046 putative cell wall protein
Cluster number: 36
Cluster clones: 700354519H1 700352645H1
Percent of clones expressed: 0.21
Cluster number: 174
Cluster clones: 700354942H1 700355507H1
Percent of clones expressed: 0.21
Cluster number: 50
Cluster clones: 700352769H1 700352953H1
Percent of clones expressed: 0.21
Homologous match to
g606810 Zea mays carbonic anhydrase mRNA, complete cds.
Cluster number: 220
Cluster clones: 700357155H1 700356835H1
Percent of clones expressed: 0.21
Cluster number: 225
Cluster clones: 700382078H1 700382273H1
Percent of clones expressed: 0.21
Cluster number: 155
Cluster clones: 700355780H1 700354516H1
Percent of clones expressed: 0.21
Weak match to
g387431 metallothionien II
Cluster number: 186
Cluster clones: 700355345H1 700355344H1
Percent of clones expressed: 0.21
Cluster number: 7
Cluster clones:
700282548H2
700282438H2
Percent of clones expressed: 0.21
Weak match to
q2695929 putative thiol protease
Cluster number: 194
Cluster clones: 700355594H1 700356974H1
Percent of clones expressed: 0.21
Weak match to
g1872473 TA-MT; delta-24-sterol methyltransferase
Cluster number: 111
Cluster clones: 700356473H1 700353560H1
Percent of clones expressed: 0.21
Homologous match to
g1002369 coatomer protein
Cluster number: 206
Cluster clones: 700356174H1 700356190H1
Percent of clones expressed: 0.21
Homologous match to
g2623961 A. graveolens mRNA for isocitrate dehydrogenase (NADP+).
Cluster number: 136
Cluster clones: 700354305H1 700355422H1
Percent of clones expressed: 0.21
Cluster number: 217
Cluster clones: 700356695H1 700382260H1
Percent of clones expressed: 0.21
Cluster number: 41
Cluster clones: 700352804H1 700352705H1
Percent of clones expressed: 0.21
Homologous match to
g1030068 NAD(P)H oxidoreductase, isoflavone reductase homologue
Cluster number: 175
Cluster clones: 700356081H1 700354954H1
Percent of clones expressed: 0.21
Cluster number: 117
Cluster clones: 700353848H1 700353614H1
Percent of clones expressed: 0.21
Homologous match to
g1710150 Arabidopsls thaliana proline iminopeptidase mRNA, complete cds.
Cluster number: 27
Cluster clones:
700282584H2
700282592H2
Percent of clones expressed: 0.21
Cluster number: 245
Cluster clones: 700382873H1 700383182H1
Percent of clones expressed: 0.21
Homologous match to
g1296806 H. vulgare xylose isomerase gene.
Cluster number: 200
Cluster clones: 700357415H1 700355871H1
Percent of clones expressed: 0.21
Cluster number: 236
Cluster clones: 700382902H1 700382425H1
Percent of clones expressed: 0.21
Homologous match to
g1649012 egg receptor for sperm
Cluster number: 120
Cluster clones: 700353713H1 700354534H1
Percent of clones expressed: 0.21
Cluster number: 195
Cluster clones: 700355966H1 700355766H1
Percent of clones expressed: 0.21
Weak match to
g2832707 T9A21.180; translation initiation factor eIF-2 gamma chain-like
protein
Cluster number: 207
Cluster clones: 700356177H1 700356193H1
Percent of clones expressed: 0.21
Weak match to
g2828188 Arabidopsis thaliana genomic DNA, chromosome 5, TAC clone: K3I3,
complete sequence.
Cluster number: 108
Cluster clones: 700355518H1 700353516H1
Percent of clones expressed: 0.21
Homologous match to
g2384676 Arabidopsis thaliana putative potassium transporter AtKT5p (AtKT5)
mRNA, partial cds.
Cluster number: 65
Cluster clones: 700352935H1 700354658H1
Percent of clones expressed: 0.21
Cluster number: 166
Cluster clones: 700356767H1 700354769H1
Percent of clones expressed: 0.21
Cluster number: 101
Cluster clones: 700354301H1 700353262H1
Percent of clones expressed: 0.21
Homologous match to
g2370231 Hordeum vulgare mRNA for putative acyl-CoA oxidase.
Cluster number: 238
Cluster clones: 700382439H1 700382450H1
Percent of clones expressed: 0.21
Weak match to
g35597 PR 264
Cluster number: 246
Cluster clones: 700383019H1 700383011H1
Percent of clones expressed: 0.21
Cluster number: 132
Cluster clones: 700355447H1 700354247H1
Percent of clones expressed: 0.21
Cluster number: 168
Cluster clones: 700354777H1 700354793H1
Percent of clones expressed: 0.21
Cluster number: 210
Cluster clones: 700356541H1 700356241H1
Percent of clones expressed: 0.21
Cluster number: 140
Cluster clones: 700354446H1 700354326H1
Percent of clones expressed: 0.21
Cluster number: 24
Cluster clones:
70028256432 700356229H1
Percent of clones expressed: 0.21
Homologous match to
g939778 Zea mays MADS-box protein (ZAG3) mRNA, complete cds.
Cluster number: 221
Cluster clones: 700356901H1 700356933H1
Percent of clones expressed: 0.21
Weak match to
g1402878 orf4; unknown
Cluster number: 151
Cluster clones: 700354425H1 700356025H1
Percent of clones expressed: 0.21
Weak match to
g1657854 Triticum aestivum cold acclimation protein WCOR413 (Wcor4l3) mRNA,
complete cds.
Cluster number: 98
Cluster clones: 700353215H1 700356847H1
Percent of clones expressed: 0.21
Weak match to
g288611 Z. mays PG gene for polygalacturonase.
Cluster number: 138
Cluster clones: 700383051H1 700354310H1
Percent of clones expressed: 0.21
Cluster number: 149
Cluster clones: 700354409H1 700355040H1
Percent of clones expressed: 0.21
Cluster number: 230
Cluster clones: 700382365H1 700382210H1
Percent of clones expressed: 0.21
Cluster number: 160
Cluster clones: 700354623H1 700354655H1
Percent of clones expressed: 0.21
Homologous match to
g2529228 Clycine max mRNA for 6-phosphogluconate dehydrogenase, complete
cds.
Cluster number: 55
Cluster clones: 700352838H1 700352825H1
Percent of clones expressed: 0.21
Homologous match to
g2252856 A_TM018A10.8
Cluster number: 219
Cluster clones: 700382882H1 700356796H1
Percent of clones expressed: 0.21
Weak match to
g454880 Rice mRNA for WSI724 protein induced by water stress, complete
cds.
Cluster number: 177
Cluster clones: 700357173H1 700355011H1
Percent of clones expressed: 0.21
Cluster number: 119
Cluster clones: 700355989H1 700353653H1
Percent of clones expressed: 0.21
Cluster number: 247
Cluster clones: 700383023H1 700383015H1
Percent of clones expressed: 0.21
Cluster number: 241
Cluster clones: 700382542H1 700382643H1
Percent of clones expressed: 0.21
Homologous match to
g1899187 Nicotiana tabacum DNA binding protein ACBF mRNA, complete cds.
Cluster number: 211
Cluster clones: 700356252H1 700356551H1
Percent of clones expressed: 0.21
Cluster number: 228
Cluster clones: 700382123H1 700383147H1
Percent of clones expressed: 0.21
Cluster number: 158
Cluster clones: 700355758H1 700354558H1
Percent of clones expressed: 0.21
Cluster number: 180
Cluster clones: 700355173H1 700355067H1
Percent of clones expressed: 0.21
Cluster number: 21
Cluster clones:
700282550H2 700356152H1
Percent of clones expressed: 0.21
Cluster number: 113
Cluster clones: 700353567H1 700382302H1
Percent of clones expressed: 0.21
Homologous match to
g1532167 AT.I.24-5
Cluster number: 129
Cluster clones: 700353986H1 700354709H1
Percent of clones expressed: 0.21
Homologous match to
g2330689 Yeast (Saccharomyces pombe) chromosome I cosmid c16E8.
Cluster number: 64
Cluster clones: 700383037H1 700352916H1
Percent of clones expressed: 0.21
Weak match to
g1532047 O. sativa mRNA for S-adenosylmethionine decarboxylase.
Cluster number: 209
Cluster clones: 700356848H1 700356236H1
Percent of clones expressed: 0.21
Weak match to
g2160155 Sequence of BAC F21M12 from Arabidopsis thaliana chromosome 1,
complete sequence.
Cluster number: 139
Cluster clones: 700354317H1 700355077H1
Percent of clones expressed: 0.21
Weak match to
g2342495 Ananas comosus mRNA for bromelain, complete cds.
Cluster number: 197
Cluster clones: 700356076H1 700355829H1
Percent of clones expressed: 0.21
Weak match to
g168557 Zea mays putative GTP-binding protein homolog mRNA, partial cds.
Cluster number: 161
Cluster clones: 700356624H1 700354637H1
Percent of clones expressed: 0.21
Cluster number: 231
Cluster clones: 700382220H1 700382268H1
Percent of clones expressed: 0.21
Weak match to
g2160144 Strong similarity to Arabidopsis oligopeptide transporter
(gb|X77503)
Cluster number: 248
Cluster clones: 700383166H1 700383061H1
Percent of clones expressed: 0.21
Cluster number: 222
Cluster clones: 700356903H1 700356935H1
Percent of clones expressed: 0.21
Cluster number: 178
Cluster clones: 700355022H1 700382176H1
Percent of clones expressed: 0.21
Cluster number: 170
Cluster clones: 700354795H1 700354779H1
Percent of clones expressed: 0.21
Homologous match to
g2827700 P6Hl1.20; DEAD box ATP dependent helicase protein
Cluster number: 239
Cluster clones: 700382513H1 700382654H1
Percent of clones expressed: 0.21
Cluster number: 212
Cluster clones: 700356273H1 700352891H1
Percent of clones expressed: 0.21
Weak match to
g12956 reading frame protein 5 (896 is 2nd base in codon)
Cluster number: 159
Cluster clones: 700356802H1 700354606H1
Percent of clones expressed: 0.21
Cluster number: 169
Cluster clones: 700354794H1 700354778H1
Percent of clones expressed: 0.21
Homologous match to
g1438861 GmCK2p; EC 2.7.1.32
Cluster number: 123
Cluster clones: 700353839H1 700356483H1
Percent of clones expressed: 0.21
Cluster number: 198
Cluster clones: 700355856H1 700355888H1
Percent of clones expressed: 0.21
Weak match to
g2330673 rp122; 60S ribosomal protein
Cluster number: 240
Cluster clones: 700382649H1 700382537H1
Percent of clones expressed: 0.21
Weak match to
g469232 vacuolar H+-ATPase subunit
Cluster number: 104
Cluster clones: 700356062H1 700353279H1
Percent of clones expressed: 0.21
Cluster number: 162
Cluster clones: 700355770H1 700354638H1
Percent of clones expressed: 0.21
Homologous match to
g2213424 Citrus paradisi mRNA for hypothetical protein.
Cluster number: 191
Cluster clones: 700355496H1 700382665H1
Percent of clones expressed: 0.21
Cluster number: 69
Cluster clones: 700354930H1 700352961H1
Percent of clones expressed: 0.21
Cluster number: 232
Cluster clones: 700382226H1 700382307H1
Percent of clones expressed: 0.21
Homologous match to
g2702272 T21L14.13
Cluster number: 76
Cluster clones: 700356644H1 7003530l2Hl
Percent of clones expressed: 0.21
Cluster number: 179
Cluster clones: 700356265H1 700355065H1
Percent of clones expressed: 0.21
Cluster number: 249
Cluster clones: 700383075H1 700383157H1
Percent of clones expressed: 0.21
Homologous match to
g1814402 Mesembryanthemum crystallinum methionine synthase (MetE) mRNA,
complete cds.
Cluster number: 213
Cluster clones: 700356468H1 700382579H1
Percent of clones expressed: 0.21
Weak match to
g498747 Pir7b
Cluster number: 1
Cluster clones:
700282401H2
700282591H2
Percent of clones expressed: 0.21
Weak match to
g409499 3-Hydroxy-3-methylglutaryl coenzyme A lyase, HL {EC 4.1.3.4}
[mice, liver, Peptide Mitochondrial, 325 aa]
Cluster number: 16
Cluster clones:
700282522H2
700282514H2
Percent of clones expressed: 0.21
Cluster number: 35
Cluster clones: 700355842H1 700352636H1
Percent of clones expressed: 0.21
Weak match to
g2209343 copper transport protein Atox1
Cluster number: 23
Cluster clones:
700282561H2
700282562H2
Percent of clones expressed: 0.21
Cluster number: 182
Cluster clones: 700355286H1 700383026H1
Percent of clones expressed: 0.21
Cluster number: 199
Cluster clones: 700355861H1 700355866H1
Percent of clones expressed: 0.21
Cluster number: 242
Cluster clones: 700382834H1 700382554H1
Percent of clones expressed: 0.21
Cluster number: 233
Cluster clones: 700383043H1 700382324H1
Percent of clones expressed: 0.21
Cluster number: 88
Cluster clones: 700353107H1 700356032H1
Percent of clones expressed: 0.21
Homologous match to
g456567 Pisum sativum ubiquitin conjugating enzyme (UBC4), complete cds.
Cluster number: 73
Cluster clones: 700352993H1 700355070H1
Percent of clones expressed: 0.21
Cluster number: 95
Cluster clones: 700356702H1 700353201H1
Percent of clones expressed: 0.21
Cluster number: 189
Cluster clones: 700355456H1 700356821H1
Percent of clones expressed: 0.21
Homologous match to
g1498325 Glycine max actin (Soy86) gene, partial cds.
Cluster number: 223
Cluster clones: 700382636H1 700357183H1
Percent of clones expressed: 0.21
Weak match to
g2584827 Sequence of BAC Fl2F1 from Arabidopsis thaliana chromosome 1,
complete sequence.
Cluster number: 153
Cluster clones: 700355708H1 700354508H1
Percent of clones expressed: 0.21
Cluster number: 61
Cluster clones: 700352903H1 700382354H1
Percent of clones expressed: 0.21
Cluster number: 54
Cluster clones: 700382424H1 700352802H1
Percent of clones expressed: 0.21
Weak match to
g2226414 hypothetical protein
Cluster number: 18
Cluster clones:
70028253252
70028252152
Percent of clones expressed: 0.21
Homologous match to
g2760334 F1N21.5
Cluster number: 201
Cluster clones: 700355877H1 700382550H1
Percent of clones expressed: 0.21
Cluster number: 106
Cluster clones: 700382041H1 700353431H1
Percent of clones expressed: 0.21
Cluster number: 234
Cluster clones: 700382604H1 700382377H1
Percent of clones expressed: 0.21
Cluster number: 164
Cluster clones: 700356224H1 700354663H1
Percent of clones expressed: 0.21
Homologous match to
g804655 Hordeum vulgare L. beta-glucosidase (BGQ60) gene, complete cds.
Cluster number: 145
Cluster clones: 700354366H1 700356927H1
Percent of clones expressed: 0.21
Homologous match to
g498040 ORF
Cluster number: 215
Cluster clones: 700357194H1 700356542H1
Percent of clones expressed: 0.21
Cluster number: 56
Cluster clones: 700352826H1 700354918H1
Percent of clones expressed: 0.21
Homologous match to
g287830 polygalacturonase
Cluster number: 49
Cluster clones: 700352760H1 700356015H1
Percent of clones expressed: 0.21
Cluster number: 70
Cluster clones: 700352967H1 700356167H1
Percent of clones expressed: 0.21
Cluster number: 134
Cluster clones: 700355815H1 700354260H1
Percent of clones expressed: 0.21
Homologous match to
g1145627 lipase
Cluster number: 184
Cluster clones: 700382773H1 700355307H1
Percent of clones expressed: 0.21
Weak match to
g2351070 Arabidopsis thaliana genomic DNA, chromosome 5, P1 clone: MTH12.
Cluster number: 204
Cluster clones: 700356984H1 700356047H1
Percent of clones expressed: 0.21
Cluster number: 22
Cluster clones:
700282554H2
700282553H2
Percent of clones expressed: 0.21
Weak match to
g2463582 precursor of cysteine proteinase; FB31 precursor (FB13 precursor)
Cluster number: 235
Cluster clones: 700382401H1 700382494H1
Percent of clones expressed: 0.21
Cluster number: 165
Cluster clones: 700354678H1 700356591H1
Percent of clones expressed: 0.21
Cluster number: 243
Cluster clones: 700382687H1 700383087H1
Percent of clones expressed: 0.21
Homologous match to
g699510 M(1)1B; minute(1)1B protein; minute(1)1B
Cluster number: 115
Cluster clones: 700353590H1 700355093H1
Percent of clones expressed: 0.21
Cluster number: 94
Cluster clones: 700353195H1 700356014H1
Percent of clones expressed: 0.21
Cluster number: 131
Cluster clones: 700354244H1 700355444H1
Percent of clones expressed: 0.21
Cluster number: 146
Cluster clones: 700354371H1 700383009H1
Percent of clones expressed: 0.21
Cluster number: 110
Cluster clones: 700382240H1 700353546H1
Percent of clones expressed: 0.21
Homologous match to
g1051108 A. thaliana mRNA for adenine nucleotide transiocase.
Cluster number: 60
Cluster clones: 700352888H1 700382460H1
Percent of clones expressed: 0.21
Homologous match to
g1872521 LSD1; negative regulator of cell death; zinc-finger protein Lsd1
Cluster number: 46
Cluster clones: 700352751H1 700382640H1
Percent of clones expressed: 0.21
Homologous match to
g1321917 su12; cytoplasmic ribosomal protein S7
Cluster number: 167
Cluster clones: 700354774H1 700354790H1
Percent of clones expressed: 0.21
Cluster number: 127
Cluster clones: 700356544H1 700353924H1
Percent of clones expressed: 0.21
Homologous match to
g2264304 Arabidopsis thaliana genomic DNA, chromosome 5, P1 clone: MBG8,
complete sequence.

What is claimed is:

1. A purified corn tassel-derived polynucleotide comprising a nucleic acid sequence selected from the Sequence Listing or a complement of the nucleic acid sequence.

2. A composition for the detection of altered expression of corn tassel-derived polynucleotides comprising at least one of the nucleic acid sequences of claim 1.

3. A corn tassel-derived polynucleotide selected from the following nucleic acid sequences of claim 1: SEQ ID NO: 1615 (700344704H1), SEQ ID NO: 2691 (700346502H1), SEQ ID NO: 2813 (700346702H1), SEQ ID NO: 3616 (700380601H1), SEQ ID NO: 3681 (700380774H1), SEQ ID NO: 233 (700342388H1), SEQ ID NO: 2060 (700345470H1), SEQ ID NO: 964 (700343568H1), SEQ ID NO: 2811 (700346695H1), SEQ ID NO: 2519 (700346233H1), SEQ ID NO: 2083 (700345510H1), SEQ ID NO: 1101 (700343780H1), SEQ ID NO: 1847 (700345052H1), SEQ ID NO: 1777 (700344946H1), SEQ ID NO: 1780 (700344949H1), SEQ ID NO: 1733 (700344870H1), SEQ ID NO: 3018 (700347036H1), SEQ ID NO: 3158 (700347281H1), SEQ ID NO: 2220 (700345749H1), SEQ ID NO: 165 (700342268H1), SEQ ID NO: 139 (700342223H1), SEQ ID NO: 2823 (700346721H1), SEQ ID NO: 2065 (700345477H1), SEQ ID NO: 605 (700343040H1), SEQ ID NO: 3618 (700380603H1), SEQ ID NO: 1537 (700344525H1), SEQ ID NO: 469 (700342790H1), SEQ ID NO: 3344 (700379867H1), SEQ ID NO: 180 (700342291H1), SEQ ID NO: 303 (700342521H1), SEQ ID NO: 1249 (700344013H1), SEQ ID NO: 1036 (700343679H1), SEQ ID NO: 2920 (700346877H1), SEQ ID NO: 3619 (700380604H1), SEQ ID NO: 1061 (700343721H1), SEQ ID NO: 1589 (700344654H1), SEQ ID NO: 3100 (700347193H1), SEQ ID NO: 2182 (700345675H1), SEQ ID NO: 2218 (700345743H1), SEQ ID NO: 398 (700342679H1), SEQ ID NO: 175 (700342283H1), SEQ ID NO: 3533 (700380411H1), SEQ ID NO: 3615 (700380596H1), SEQ ID NO: 89 (700281941H2), SEQ ID NO: 3530 (700380405H1), SEQ ID NO: 3023 (700347043H1), SEQ ID NO: 3185 (700347339H1), SEQ ID NO: 3062 (700347112H1), SEQ ID NO: 1082 (700343752H1), SEQ ID NO: 55 (700281888H2), SEQ ID NO: 2955 (700346930H1), SEQ ID NO: 1032 (700343674H1), SEQ ID NO: 163 (700342263H1), SEQ ID NO: 2864 (700346807H1), SEQ ID NO: 1286 (700344069H1), SEQ ID NO: 2560 (700346291H1), SEQ ID NO: 742 (700343247H1), SEQ ID NO: 1731 (700344866H1), SEQ ID NO: 370 (700342630H1), SEQ ID NO: 1167 (700343872H1), SEQ ID NO: 715 (700343202H1), SEQ ID NO: 3314 (700379793H1), SEQ ID NO: 2156 (700345638H1), SEQ ID NO: 624 (700343071H1), SEQ ID NO: 545 (700342924H1), SEQ ID NO: 2855 (700346794H1), SEQ ID NO: 850 (700343408H1), SEQ ID NO: 1557 (700344614H1), SEQ ID NO: 1872 (700345114H1), SEQ ID NO: 1000 (700343626H1), SEQ ID NO: 1045 (700343695H1), SEQ ID NO: 2753 (700346604H1), SEQ ID NO: 70 (700281910H1), SEQ ID NO: 1235 (700343988H1), SEQ ID NO: 389 (700342664H1), SEQ ID NO: 486 (700342817H1), SEQ ID NO: 766 (700343281H1), SEQ ID NO: 1310 (700344105H1), SEQ ID NO: 3202 (700347368H1), SEQ ID NO: 1941 (700345253H1), SEQ ID NO: 6 (700281814H2), SEQ ID NO: 1884 (700345138H1), SEQ ID NO: 2316 (700345916H1), SEQ ID NO: 1948 (700345263H1), SEQ ID NO: 2277 (700345849H1), SEQ ID NO: 2019 (700345394H1), SEQ ID NO: 1696 (700344820H1), SEQ ID NO: 226 (700342373H1), SEQ ID NO: 262 (700342448H1), SEQ ID NO: 1377 (700344208H1), SEQ ID NO: 1279 (700344059H1), SEQ ID NO: 1770 (700344935H1), SEQ ID NO: 532 (700342903H1), SEQ ID NO: 843 (700343396H1), SEQ ID NO: 360 (700342616H1), SEQ ID NO: 540 (700342915H1), SEQ ID NO: 413 (700342708H1), SEQ ID NO: 1252 (700344017H1), SEQ ID NO: 1560 (700344618H1), SEQ ID NO: 3541 (700380432H1), SEQ ID NO: 243 (700342413H1), SEQ ID NO: 1938 (700345249H1), SEQ ID NO: 1127 (700343816H1), SEQ ID NO: 1545 (700344541H1), SEQ ID NO: 1946 (700345258H1), SEQ ID NO: 298 (700342510H1), SEQ ID NO: 2906 (700346860H1), SEQ ID NO: 783 (700343310H1), SEQ ID NO: 616 (700343060H1), SEQ ID NO: 2097 (700345529H1), SEQ ID NO: 2393 (700346036H1), SEQ ID NO: 860 (700343420H1), SEQ ID NO: 2196 (700345705H1), SEQ ID NO: 2403 (700346049H1), SEQ ID NO: 2309 (700345906H1), SEQ ID NO: 1963 (700345289H1), SEQ ID NO: 1383 (700344218H1), SEQ ID NO: 3201 (700347367H1), SEQ ID NO: 3589 (700380530H1), SEQ ID NO: 660 (700343124H1), SEQ ID NO: 2254 (700345807H1), SEQ ID NO: 2689 (700346496H1), SEQ ID NO: 3166 (700347296H1), SEQ ID NO: 2023 (700345404H1), SEQ ID NO: 726 (700343218H1), SEQ ID NO: 363 (700342620H1), SEQ ID NO: 2968 (700346951H1), SEQ ID NO: 2214 (700345737H1), SEQ ID NO: 989 (700343607H1), SEQ ID NO: 2642 (700346426H1), SEQ ID NO: 2741 (700346581H1), SEQ ID NO: 1321 (700344122H1), SEQ ID NO: 1255 (700344022H1), SEQ ID NO: 2311 (700345908H1), SEQ ID NO: 1857 (700345075H1), SEQ ID NO: 2571 (700346316H1), SEQ ID NO: 3063 (700347116H1), SEQ ID NO: 1649 (700344750H1), SEQ ID NO: 1653 (700344754H1), SEQ ID NO: 1762 (700344924H1), SEQ ID NO: 1985 (700345329H1), SEQ ID NO: 2382 (700346023H1), SEQ ID NO: 2468 (700346152H1), SEQ ID NO: 1567 (700344628H1), SEQ ID NO: 991 (700343609H1), SEQ ID NO: 2124 (700345577H1), SEQ ID NO: 829 (700343377H1), SEQ ID NO: 3623 (700380616H1), SEQ ID NO: 725 (700343216H1), SEQ ID NO: 1676 (700344785H1), SEQ ID NO: 399 (700342680H1), SEQ ID NO: 2790 (700346658H1), SEQ ID NO: 2284 (700345859H1), SEQ ID NO: 2838 (700346745H1), SEQ ID NO: 757 (700343266H1), SEQ ID NO: 1251 (700344016H1), SEQ ID NO: 207 (700342338H1), SEQ ID NO: 3515 (700380270H1), SEQ ID NO: 3224 (700379610H1), SEQ ID NO: 2958 (700346935H1), SEQ ID NO: 1861 (700345079H1), SEQ ID NO: 3052 (700347092H1), SEQ ID NO: 2542 (700346264H1), SEQ ID NO: 3602 (700380571H1), SEQ ID NO: 2788 (700346656H1), SEQ ID NO: 1966 (700345293H1), SEQ ID NO: 1813 (700345004H1), SEQ ID NO: 573 (700342986H1), SEQ ID NO: 3572 (700380488H1), SEQ ID NO: 1368 (700344191H1), SEQ ID NO: 774 (700343291H1), SEQ ID NO: 443 (700342747H1), SEQ ID NO: 1083 (700343755H1), SEQ ID NO: 110 (700281973H1), SEQ ID NO: 118 (700281986H2), SEQ ID NO: 2088 (700345516H1), SEQ ID NO: 2932 (700346894H1), SEQ ID NO: 2815 (700346704H1), SEQ ID NO: 1112 (700343793H1), SEQ ID NO: 3131 (700347236H1), SEQ ID NO: 682 (700343154H1), SEQ ID NO: 1965 (700345291H1), SEQ ID NO: 1866 (700345094H1), SEQ ID NO: 898 (700343475H1), SEQ ID NO: 3582 (700380514H1), SEQ ID NO: 916 (700343509H1), SEQ ID NO: 188 (700342309H1), SEQ ID NO: 1364 (700344186H1), SEQ ID NO: 1421 (700344295H1), SEQ ID NO: 3685 (700380786H1), SEQ ID NO: 2404 (700346050H1), SEQ ID NO: 3246 (700379650H1), SEQ ID NO: 1673 (700344782H1), SEQ ID NO: 953 (700343552H1), SEQ ID NO: 2216 (700345739H1), SEQ ID NO: 1232 (700343985H1), SEQ ID NO: 775 (700343292H1), SEQ ID NO: 869 (700343431H1), SEQ ID NO: 145 (700342231H1), SEQ ID NO: 2927 (700346887H1), SEQ ID NO: 1820 (700345013H1), SEQ ID NO: 2244 (700345785H1), SEQ ID NO: 1078 (700343746H1), SEQ ID NO: 2965 (700346943H1), SEQ ID NO: 1085 (700343757H1), SEQ ID NO: 730 (700343225H1), SEQ ID NO: 792 (700343325H1), SEQ ID NO: 3218 (700347395H1), SEQ ID NO: 3033 (700347064H1), SEQ ID NO: 1058 (700343715H1), SEQ ID NO: 302 (700342515H1), SEQ ID NO: 2384 (700346026H1), SEQ ID NO: 1220 (700343965H1), SEQ ID NO: 1883 (700345136H1), SEQ ID NO: 1928 (700345235H1), SEQ ID NO: 2598 (700346351H1), SEQ ID NO: 315 (700342543H1), SEQ ID NO: 1405 (700344265H1), SEQ ID NO: 3128 (700347232H1), SEQ ID NO: 942 (700343540H1), SEQ ID NO: 1109 (700343790H1), SEQ ID NO: 1490 (700344425H1), SEQ ID NO: 3040 (700347072H1), SEQ ID NO: 2411 (700346060H1), SEQ ID NO: 2477 (700346167H1), SEQ ID NO: 446 (700342755H1), SEQ ID NO: 3026 (700347050H1), SEQ ID NO: 3554 (700380452H1), SEQ ID NO: 3465 (700380146H1), SEQ ID NO: 143 (700342228H1), SEQ ID NO: 867 (700343428H1), SEQ ID NO: 1139 (700343832H1), SEQ ID NO: 3067 (700347126H1), SEQ ID NO: 799 (700343333H1), SEQ ID NO: 2059 (700345469H1), SEQ ID NO: 2120 (700345571H1), SEQ ID NO: 2179 (700345671H1), SEQ ID NO: 2707 (700346525H1), SEQ ID NO: 967 (700343572H1), SEQ ID NO: 1356 (700344174H1), SEQ ID NO: 2896 (700346848H1), SEQ ID NO: 304 (700342523H1), SEQ ID NO: 1063 (700343723H1), SEQ ID NO: 560 (700342959H1), SEQ ID NO: 2625 (700346389H1), SEQ ID NO: 2501 (700346210H1), SEQ ID NO: 319 (700342551H1), SEQ ID NO: 3173 (700347308H1), SEQ ID NO: 2313 (700345910H1), SEQ ID NO: 2022 (700345403H1), SEQ ID NO: 1076 (700343742H1), SEQ ID NO: 3025 (700347049H1), SEQ ID NO: 1719 (700344849H1), SEQ ID NO: 3342 (700379862H1), SEQ ID NO: 643 (700343103H1), SEQ ID NO: 3649 (700380682H1), SEQ ID NO: 3613 (700380589H1), SEQ ID NO: 807 (700343342H1), SEQ ID NO: 1760 (700344920H1), SEQ ID NO: 2558 (700346288H1), SEQ ID NO: 492 (700342832H1), SEQ ID NO: 504 (700342846H1), SEQ ID NO: 172 (700342278H1), SEQ ID NO: 1738 (700344878H1), SEQ ID NO: 2014 (700345384H1), SEQ ID NO: 390 (700342666H1), SEQ ID NO: 401 (700342682H1), SEQ ID NO: 3512 (700380263H1), SEQ ID NO: 3441 (700380096H1), SEQ ID NO: 2370 (700346008H1), SEQ ID NO: 1323 (700344124H1), SEQ ID NO: 634 (700343087H1), SEQ ID NO: 1986 (700345330H1), SEQ ID NO: 238 (700342403H1), SEQ ID NO: 987 (700343603H1), SEQ ID NO: 273 (700342463H1), SEQ ID NO: 208 (700342340H1), SEQ ID NO: 2674 (700346475H1), SEQ ID NO: 3119 (700347222H1), SEQ ID NO: 2203 (700345716H1), SEQ ID NO: 2186 (700345686H1), SEQ ID NO: 1307 (700344096H1), SEQ ID NO: 1372 (700344196H1), SEQ ID NO: 3053 (700347093H1), SEQ ID NO: 2507 (700346218H1), SEQ ID NO: 1049 (700343703H1), SEQ ID NO: 391 (700342667H1), SEQ ID NO: 3092 (700347176H1), SEQ ID NO: 3389 (700379975H1), SEQ ID NO: 326 (700342560H1), SEQ ID NO: 3605 (700380575H1), SEQ ID NO: 2946 (700346918H1), SEQ ID NO: 907 (700343487H1), SEQ ID NO: 3366 (700379925H1), SEQ ID NO: 1237 (700343991H1), SEQ ID NO: 3610 (700380584H1), SEQ ID NO: 2464 (700346146H1), SEQ ID NO: 3650 (700380683H1), SEQ ID NO: 2024 (700345405H1), SEQ ID NO: 1145 (700343841H1), SEQ ID NO: 1590 (700344655H1), SEQ ID NO: 2219 (700345747H1), SEQ ID NO: 224 (700342363H1), SEQ ID NO: 1108 (700343789H1), SEQ ID NO: 344 (700342589H1), SEQ ID NO: 2995 (700346996H1), SEQ ID NO: 3282 (700379719H1), SEQ ID NO: 1826 (700345020H1), SEQ ID NO: 1443 (700344334H1), SEQ ID NO: 3021 (700347040H1), SEQ ID NO: 558 (700342952H1), SEQ ID NO: 2292 (700345873H1), SEQ ID NO: 2030 (700345412H1), SEQ ID NO: 1005 (700343632H1), SEQ ID NO: 254 (700342432H1), SEQ ID NO: 1506 (700344454H1), SEQ ID NO: 1146 (700343842H1), SEQ ID NO: 1756 (700344911H1), SEQ ID NO: 2471 (700346157H1), SEQ ID NO: 1170 (700343875H1), SEQ ID NO: 1765 (700344928H1), SEQ ID NO: 1585 (700344649H1), SEQ ID NO: 495 (700342835H1), SEQ ID NO: 392 (700342670H1), SEQ ID NO: 5179 (700355947H1), SEQ ID NO: 4225 (700353526H1), SEQ ID NO: 5639 (700382063H1), SEQ ID NO: 3797 (700352605H1), SEQ ID NO: 4188 (700353271H1), SEQ ID NO: 5940 (700382691H1), SEQ ID NO: 4388 (700353955H1), SEQ ID NO: 5962 (700382739H1), SEQ ID NO: 6034 (700382890H1), SEQ ID NO: 4988 (700355520H1), SEQ ID NO: 5105 (700355788H1), SEQ ID NO: 5943 (700382702H1), SEQ ID NO: 4276 (700353625H1), SEQ ID NO: 5267 (700356140H1), SEQ ID NO: 3698 (700282414H2), SEQ ID NO: 4942 (700355431H1), SEQ ID NO: 4803 (700355029H1), SEQ ID NO: 5171 (700355925H1), SEQ ID NO: 5366 (700356466H1), SEQ ID NO: 4940 (700355428H1), SEQ ID NO: 5895 (700382605H1), SEQ ID NO: 4403 (700354129H1), SEQ ID NO: 4830 (700355088H1), SEQ ID NO: 5695 (700382183H1), SEQ ID NO: 5440 (700356708H1), SEQ ID NO: 5526 (700357108H1), SEQ ID NO: 5529 (700357122H1), SEQ ID NO: 5371 (700356474H1), SEQ ID NO: 4029 (700353005H1), SEQ ID NO: 5099 (700355779H1), SEQ ID NO: 4100 (700353128H1), SEQ ID NO: 3790 (700282575H2), SEQ ID NO: 3835 (700352675H1), SEQ ID NO: 3943 (700352843H1), SEQ ID NO: 3856 (700352709H1), SEQ ID NO: 3874 (700352735H1), SEQ ID NO: 4959 (700355462H1), SEQ ID NO: 5010 (700355558H1), SEQ ID NO: 4336 (700353828H1), SEQ ID NO: 5420 (700356627H1), SEQ ID NO: 5751 (700382275H1), SEQ ID NO: 5301 (700356219H1), SEQ ID NO: 6184 (700383158H1), SEQ ID NO: 5003 (700355547H1), SEQ ID NO: 4800 (700355026H1), SEQ ID NO: 4610 (700354647H1), SEQ ID NO: 4789 (700355009H1), SEQ ID NO: 4466 (700354337H1), SEQ ID NO: 5278 (700356158H1), SEQ ID NO: 4742 (700354908H1), SEQ ID NO: 5260 (700356129H1), SEQ ID NO: 5661 (700382106H1), SEQ ID NO: 3806 (700352616H1), SEQ ID NO: 4555 (700354518H1), SEQ ID NO: 5232 (700356066H1), SEQ ID NO: 5216 (700356023H1), SEQ ID NO: 6002 (700382827H1), SEQ ID NO: 4217 (700353446H1), SEQ ID NO: 4793 (700355016H1), SEQ ID NO: 4204 (700353409H1), SEQ ID NO: 6178 (700383149H1), SEQ ID NO: 6083 (700382979H1), SEQ ID NO: 4876 (700355249H1), SEQ ID NO: 4031 (700353007H1), SEQ ID NO: 6098 (700383013H1), SEQ ID NO: 6102 (700383021H1), SEQ ID NO: 4421 (700354239H1), SEQ ID NO: 5946 (700382708H1), SEQ ID NO: 5214 (700356020H1), SEQ ID NO: 5703 (700382195H1), SEQ ID NO: 3750 (700282503H2), SEQ ID NO: 4212 (700353432H1), SEQ ID NO: 6090 (700382995H1), SEQ ID NO: 4713 (700354850H1), SEQ ID NO: 4197 (700353284H1), SEQ ID NO: 5692 (700382179H1), SEQ ID NO: 4727 (700354875H1), SEQ ID NO: 5924 (700382668H1), SEQ ID NO: 4052 (700353052H1), SEQ ID NO: 4111 (700353142H1), SEQ ID NO: 4043 (700353028H1), SEQ ID NO: 5723 (700382229H1), SEQ ID NO: 5560 (700357402H1), SEQ ID NO: 5603 (700357489H1), SEQ ID NO: 5700 (70038219151), SEQ ID NO: 5875 (700382543H1), SEQ ID NO: 3811 (700352625H1), SEQ ID NO: 6081 (700382975H1), SEQ ID NO: 5186 (700355963H1), SEQ ID NO: 5813 (700382392H1), SEQ ID NO: 5155 (700355889H1), SEQ ID NO: 4632 (700354689H1), SEQ ID NO: 5674 (700382139H1), SEQ ID NO: 4279 (700353629H1), SEQ ID NO: 4625 (700354676H1), SEQ ID NO: 5586 (700357445H1), SEQ ID NO: 4054 (700353057H1), SEQ ID NO: 5724 (700382230H1), SEQ ID NO: 5699 (700382190H1), SEQ ID NO: 6181 (700383154H1), SEQ ID NO: 3982 (700352912H1), SEQ ID NO: 4654 (700354742H1), SEQ ID NO: 4737 (700354895H1), SEQ ID NO: 4321 (700353779H1), SEQ ID NO: 4934 (700355415H1), SEQ ID NO: 5543 (700357160H1), SEQ ID NO: 4933 (700355414H1), SEQ ID NO: 3713 (700282435H1), SEQ ID NO: 3787 (700282569H2), SEQ ID NO: 3789 (700282571H2), SEQ ID NO: 4814 (700355053H1), SEQ ID NO: 5555 (700357189H1), SEQ ID NO: 3876 (700352737H1), SEQ ID NO: 4208 (700353424H1), SEQ ID NO: 3827 (700352654H1), SEQ ID NO: 3863 (700352719H1), SEQ ID NO: 6130 (700383062H1), SEQ ID NO: 4041 (700353023H1), SEQ ID NO: 5101 (700355781H1), SEQ ID NO: 5792 (700382353H1), SEQ ID NO: 4983 (700355513H1), SEQ ID NO: 4089 (700353114H1), SEQ ID NO: 4348 (700353853H1), SEQ ID NO: 6182 (700383155H1), SEQ ID NO: 5716 (700382218H1), SEQ ID NO: 5102 (700355783H1), SEQ ID NO: 5859 (700382507H1), SEQ ID NO: 5442 (700356725H1), SEQ ID NO: 5998 (700382817H1), SEQ ID NO: 4037 (700353014H1), SEQ ID NO: 4404 (700354142H1), SEQ ID NO: 5734 (700382245H1), SEQ ID NO: 5392 (700356534H1), SEQ ID NO: 5675 (700382140H1), SEQ ID NO: 5746 (700382267H1), SEQ ID NO: 4102 (700353130H1), SEQ ID NO: 4536 (700354478H1), SEQ ID NO: 4219 (700353509H1), SEQ ID NO: 5151 (700355885H1), SEQ ID NO: 4268 (700353612H1), SEQ ID NO: 3774 (700282549H2), SEQ ID NO: 3772 (700282547H2), SEQ ID NO: 5283 (700356171H1), SEQ ID NO: 5647 (700382075H1), SEQ ID NO: 5778 (700382330H1), SEQ ID NO: 5014 (700355577H1), SEQ ID NO: 5825 (700382426H1), SEQ ID NO: 5866 (700382525H1), SEQ ID NO: 4556 (700354519H1), SEQ ID NO: 3821 (700352645H1), SEQ ID NO: 4762 (700354942H1), SEQ ID NO: 4981 (700355507H1), SEQ ID NO: 5540 (700357155H1), SEQ ID NO: 5476 (700356835H1), SEQ ID NO: 5650 (700382078H1), SEQ ID NO: 5750 (700382273H1), SEQ ID NO: 5100 (700355780H1), SEQ ID NO: 4553 (700354516H1), SEQ ID NO: 4923 (700355345H1), SEQ ID NO: 4922 (700355344H1), SEQ ID NO: 3773 (700282548H2), SEQ ID NO: 3715 (700282438H2), SEQ ID NO: 5022 (700355594H1), SEQ ID NO: 5516 (700356974H1), SEQ ID NO: 4451 (700354305H1), SEQ ID NO: 4938 (700355422H1), SEQ ID NO: 5436 (700356695H1), SEQ ID NO: 5742 (700382260H1), SEQ ID NO: 5242 (700356081H1), SEQ ID NO: 4766 (700354954H1), SEQ ID NO: 3792 (700282584H2), SEQ ID NO: 3795 (700282592H2), SEQ ID NO: 5569 (700357415H1), SEQ ID NO: 5142 (700355871H1), SEQ ID NO: 4305 (700353713H1), SEQ ID NO: 4559 (700354534H1), SEQ ID NO: 5188 (700355966H1), SEQ ID NO: 5088 (700355766H1), SEQ ID NO: 5286 (700356177H1), SEQ ID NO: 5294 (700356193H1), SEQ ID NO: 3995 (700352935H1), SEQ ID NO: 4615 (700354658H1), SEQ ID NO: 5453 (700356767H1), SEQ ID NO: 4667 (700354769H1), SEQ ID NO: 5832 (700382439H1), SEQ ID NO: 5839 (700382450H1), SEQ ID NO: 6101 (700383019H1), SEQ ID NO: 6097 (700383011H1), SEQ ID NO: 4951 (700355447H1), SEQ ID NO: 4423 (700354247H1), SEQ ID NO: 4672 (700354777H1), SEQ ID NO: 4682 (700354793H1), SEQ ID NO: 5395 (700356541H1), SEQ ID NO: 5311 (700356241H1), SEQ ID NO: 4522 (700354446H1), SEQ ID NO: 4460 (700354326H1), SEQ ID NO: 5493 (700356901H1), SEQ ID NO: 5507 (700356933H1), SEQ ID NO: 4512 (700354425H1), SEQ ID NO: 5217 (700356025H1), SEQ ID NO: 4159 (700353215H1), SEQ ID NO: 5480 (700356847H1), SEQ ID NO: 6123 (700383051H1), SEQ ID NO: 4452 (700354310H1), SEQ ID NO: 4499 (700354409H1), SEQ ID NO: 4808 (700355040H1), SEQ ID NO: 5800 (700382365H1), SEQ ID NO: 5710 (700382210H1), SEQ ID NO: 6030 (700382882H1), SEQ ID NO: 5461 (700356796H1), SEQ ID NO: 5548 (700357173H1), SEQ ID NO: 4790 (700355011H1), SEQ ID NO: 5201 (700355989H1), SEQ ID NO: 4286 (700353653H1), SEQ ID NO: 6103 (700383023H1), SEQ ID NO: 6099 (700383015H1), SEQ ID NO: 5317 (700356252H1), SEQ ID NO: 5399 (700356551H1), SEQ ID NO: 5667 (700382123H1), SEQ ID NO: 6176 (700383147H1), SEQ ID NO: 5084 (700355758H1), SEQ ID NO: 4566 (700354558H1), SEQ ID NO: 4845 (700355173H1), SEQ ID NO: 4819 (700355067H1), SEQ ID NO: 3775 (700282550H2), SEQ ID NO: 5273 (700356152H1), SEQ ID NO: 6111 (700383037H1), SEQ ID NO: 3984 (700352916H1), SEQ ID NO: 5481 (700356848H1), SEQ ID NO: 5309 (700356236H1), SEQ ID NO: 4456 (700354317H1), SEQ ID NO: 4824 (700355077H1), SEQ ID NO: 5240 (700356076H1), SEQ ID NO: 5121 (700355829H1), SEQ ID NO: 5418 (700356624H1), SEQ ID NO: 4603 (700354637H1), SEQ ID NO: 5718 (700382220H1), SEQ ID NO: 5747 (700382268H1), SEQ ID NO: 6189 (700383166H1), SEQ ID NO: 6129 (700383061H1), SEQ ID NO: 5494 (700356903H1), SEQ ID NO: 5508 (700356935H1), SEQ ID NO: 4797 (700355022H1), SEQ ID NO: 5691 (700382176H1), SEQ ID NO: 5862 (700382513H1), SEQ ID NO: 5917 (700382654H1), SEQ ID NO: 5328 (700356273H1), SEQ ID NO: 3971 (700352891H1), SEQ ID NO: 5463 (700356802H1), SEQ ID NO: 4588 (700354606H1), SEQ ID NO: 4342 (700353839H1), SEQ ID NO: 5376 (700356483H1), SEQ ID NO: 5136 (700355856H1), SEQ ID NO: 5154 (700355888H1), SEQ ID NO: 5916 (700382649H1), SEQ ID NO: 5869 (700382537H1), SEQ ID NO: 5231 (700356062H1), SEQ ID NO: 4194 (700353279H1), SEQ ID NO: 4978 (700355496H1), SEQ ID NO: 5923 (700382665H1), SEQ ID NO: 4755 (700354930H1), SEQ ID NO: 4008 (700352961H1), SEQ ID NO: 5424 (700356644H1), SEQ ID NO: 4035 (700353012H1), SEQ ID NO: 5323 (700356265H1), SEQ ID NO: 4818 (700355065H1), SEQ ID NO: 5367 (700356468H1), SEQ ID NO: 5888 (700382579H1), SEQ ID NO: 3690 (700282401H2), SEQ ID NO: 3794 (700282591H2), SEQ ID NO: 3764 (700282522H2), SEQ ID NO: 3758 (700282514H2), SEQ ID NO: 5128 (700355842H1), SEQ ID NO: 3818 (700352636H1), SEQ ID NO: 3781 (700282561H2), SEQ ID NO: 3782 (700282562H2), SEQ ID NO: 4889 (700355286H1), SEQ ID NO: 6104 (700383026H1), SEQ ID NO: 5138 (700355861H1), SEQ ID NO: 5140 (700355866H1), SEQ ID NO: 6006 (700382834H1), SEQ ID NO: 5880 (700382554H1), SEQ ID NO: 6116 (700383043H1), SEQ ID NO: 5774 (700382324H1), SEQ ID NO: 4024 (700352993H1), SEQ ID NO: 4820 (700355070H1), SEQ ID NO: 5438 (700356702H1), SEQ ID NO: 4150 (700353201H1), SEQ ID NO: 5909 (700382636H1), SEQ ID NO: 5551 (700357183H1), SEQ ID NO: 5055 (700355708H1), SEQ ID NO: 4551 (700354508H1), SEQ ID NO: 3975 (700352903H1), SEQ ID NO: 5793 (700382354H1), SEQ ID NO: 5823 (700382424H1), SEQ ID NO: 3915 (700352802H1), SEQ ID NO: 5146 (700355877H1), SEQ ID NO: 5879 (700382550H1), SEQ ID NO: 5627 (700382041H1), SEQ ID NO: 4211 (700353431H1), SEQ ID NO: 5894 (700382604H1), SEQ ID NO: 5806 (700382377H1), SEQ ID NO: 5558 (700357194H1), SEQ ID NO: 5396 (700356542H1), SEQ ID NO: 3892 (700352760H1), SEQ ID NO: 5211 (700356015H1), SEQ ID NO: 4011 (700352967H1), SEQ ID NO: 5282 (700356167H1), SEQ ID NO: 5978 (700382773H1), SEQ ID NO: 4898 (700355307H1), SEQ ID NO: 5522 (700356984H1), SEQ ID NO: 5226 (700356047H1), SEQ ID NO: 3777 (700282554H2), SEQ ID NO: 3776 (700282553H2), SEQ ID NO: 5817 (700382401H1), SEQ ID NO: 5858 (700382494H1), SEQ ID NO: 4627 (700354678H1), SEQ ID NO: 5411 (700356591H1), SEQ ID NO: 4257 (700353590H1), SEQ ID NO: 4833 (700355093H1), SEQ ID NO: 4148 (700353195H1), SEQ ID NO: 5210 (700356014H1), SEQ ID NO: 4422 (700354244H1), SEQ ID NO: 4949 (700355444H1), SEQ ID NO: 4481 (700354371H1), SEQ ID NO: 6095 (700383009H1), SEQ ID NO: 4670 (700354774H1), SEQ ID NO: 4680 (700354790H1).

4. A nucleic acid sequence complementary to at least a fragment of at least one corn tassel-derived polynucleotide of claim 1.

5. A method for detecting a polynucleotide in a biological sample, the method comprising the steps of:
a) providing a biological sample containing nucleic acids,
b) hybridizing a polynucleotide of claim 4 to nucleic acids in the biological sample, thereby forming a hybridization complex; and
c) detecting the hybridization complex, wherein the presence of the hybridization complex correlates with the presence of the polynucleotide in the sample.

6. The method of claim 5 wherein the nucleic acids of the biological sample are amplified prior to hybridization.

7. A method for using oligomers and amplification to recover a regulatory element from a DNA library, the method comprising the steps of:
a) designing oligomers to at least one of the sequences of Claim
b) combining the oligomers with a DNA library under appropriate conditions to amplify the sequence of step a);
c) comparing the nucleic acid sequence of step a) with the amplified sequence to identify overlapping areas;
d) identifying additional sequence beyond the overlapping areas, and
e) repeating steps a) through d) until the regulatory element is recovered.

8. A tassel-specific regulatory element which regulates the expression of at least one of the sequences of claim 1.

9. An expression vector containing the regulatory element of claim 8.

10. A plant containing the expression vector of claim 9.

11. An expression vector containing the corn tassel-derived polynucleotide of claim 1.

12. A host cell containing the expression vector of claim 11.

13. A method for producing a corn tassel-derived polypeptide, said method comprising the steps of:
a) culturing the host cells of claim 12 under conditions suitable for the expression of the corn tassel-derived polypeptide; and
b) recovering the corn tassel-derived polypeptide from the cell culture.

14. A purified corn tassel-derived polypeptide (CDP) encoded by at least one of the corn tassel-derived polynucleotides of claim 1.

15. An anti-CDP antibody specifically binding the purified corn tassel-derived polypeptide of claim 14.

16. A method of identifying a test compound which specifically binds to the polypeptide of claim 14, the method comprising the steps of:
a) providing a test compound;
b) combining the polypeptide with the test compound for a sufficient time and under suitable conditions for binding; and
c) detecting binding of the polypeptide to the test compound, thereby identifying the compound which specifically binds the polypeptide.

17. A method of screening a plurality of molecules for specific binding to the polynucleotide of claim 1, the method comprising the steps of:
a) providing the plurality of molecules;
b) combining the polynucleotide with each of the plurality of molecules for a time sufficient to allow binding under suitable conditions; and
c) detecting binding of the polynucleotide to each of the plurality of molecules, thereby identifying the molecules which specifically bind the polynucleotide.
